(19) United States
(12) Patent Application Publication
(10) Pub. No.: US 2024/0164210 A1
(43) Pub. Date: May 16, 2024

(54) LIGHT EMITTING ELEMENT AND NITROGEN CONTAINING COMPOUND FOR THE SAME

(71) Applicant: Samsung Display Co., LTD., Yongin-si (KR)

(72) Inventors: HYOYOUNG LEE, Yongin-si (KR); SEOWON CHO, Yongin-si (KR); YOUNGJIN PARK, Yongin-si (KR); Eunhye AN, Suwon-si (KR); HEECHOON AHN, Yongin-si (KR); HYUNAH UM, Yongin-si (KR); Yeon Sook CHUNG, Suwon-si (KR); Eunjeong CHOI, Suwon-si (KR)

(21) Appl. No.: 18/475,059

(22) Filed: Sep. 26, 2023

(30) Foreign Application Priority Data
Oct. 5, 2022 (KR) 10-2022-0127381

(51) Int. Cl. H10K 85/60; C07D 403/14; C07D 405/14; C07D 409/14; C09K 11/06

(52) U.S. Cl. CPC H10K 85/6572; C07D 403/14; C07D 405/14; C07D 409/14; C09K 11/06; H10K 85/654; H10K 85/6574; H10K 85/6576; H10K 50/12

(57) ABSTRACT

Provided is a light emitting element including a first electrode, a second electrode facing the first electrode, and an emission layer between the first electrode and the second electrode. The light emitting element according to an embodiment includes a nitrogen-containing compound represented by Formula 1 in the emission layer, and may thus exhibit improved luminous efficiency, long lifespan, and low driving voltage.

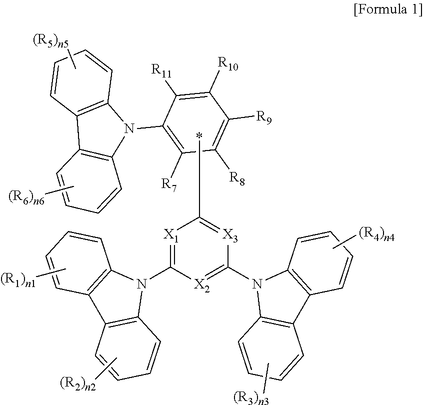

### CROSS-REFERENCE TO RELATED APPLICATIONS

This application claims priority to and the benefit of Korean Patent Application No. 10-2022-0127381, filed on Oct. 5, 2022, the entire contents of which are hereby incorporated by reference.

### BACKGROUND

### 1. Field

Embodiments of the present disclosure herein relate to a nitrogen-containing compound and a light emitting element including the same, and, for example, to a light emitting element including a novel nitrogen-containing compound in an emission layer.

### 2. Description of the Related Art

As image display devices, organic electroluminescence display devices and the like have been actively developed lately. The organic electroluminescence display devices and the like are display devices including so-called self-luminescent light emitting elements in which holes and electrons injected from a first electrode and a second electrode recombine in an emission layer, and thus a luminescent material in the emission layer emits light to accomplish display.

For application of light emitting elements to display devices, there is a demand for light emitting elements having a low driving voltage, high luminous efficiency, and a long life, and development of materials, for light emitting elements, capable of stably attaining such characteristics is being conducted substantially continuously.

### SUMMARY

Embodiments of the present disclosure provide a light emitting element having reduced driving voltage, increased luminous efficiency, and longer element service life.

Embodiments of the present disclosure also provide a nitrogen-containing compound as a material for a light emitting element, which reduces driving voltage and increases light efficiency and service life.

An embodiment of the present disclosure provides a light emitting element including a first electrode, a second electrode on the first electrode, and an emission layer between the first electrode and the second electrode and containing a first compound represented by Formula 1 below.

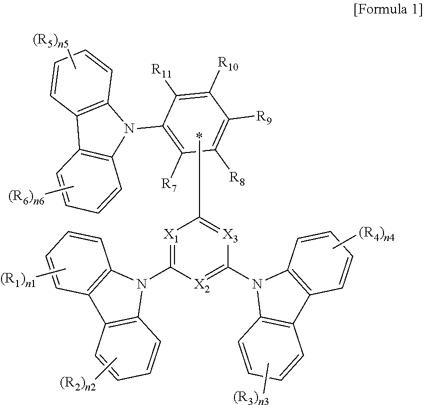

In Formula 1 above, at least one selected from X₁ to X₃ may be N, and the others may each independently be CRₓ. Rₓ may be a hydrogen atom, a deuterium atom, a substituted or unsubstituted alkyl group having 1 to 60 carbon atoms, a substituted or unsubstituted aryl group having 6 to 60 ring-forming carbon atoms, or a substituted or unsubstituted heteroaryl group having 2 to 60 ring-forming carbon atoms. At least one selected from R₁ to R₁₁ may be a deuterium atom, an unsubstituted carbazole group, a deuterium atom-substituted or unsubstituted phenyl group, an unsubstituted dibenzofuran group, an unsubstituted dibenzothiophene group, or an unsubstituted 9-phenylcarbazole group, and the others are each independently a hydrogen atom, a cyano group, a substituted or unsubstituted alkyl group having 1 to 60 carbon atoms, a substituted or unsubstituted aryl group having 6 to 60 ring-forming carbon atoms, or a substituted or unsubstituted heteroaryl group having 2 to 60 ring-forming carbon atoms, or bonded to an adjacent group to form a ring. n1 to n6 may each independently be an integer of 0 to 4, and “-*” may be connected to any one selected from among R₇ to R₁₁. In one or more embodiments, a case where in Formula 1, X₁ to X₃ are all N, “-*” is connected to R₇, R₈ is an unsubstituted carbazole group, and R₁ to R₆ and R₉ to R₁₁ are all hydrogen atoms may be excluded.

In an embodiment, the first compound represented by Formula 1 above may be represented by Formula 2 below.

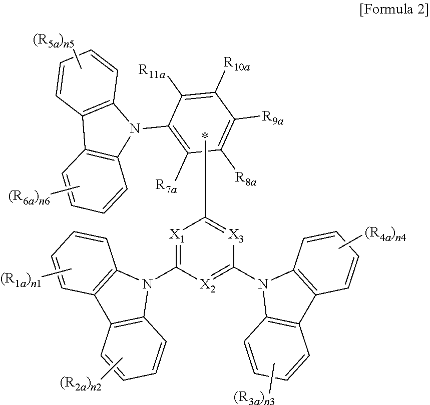

In Formula 2 above, at least one selected from R₁ₐ to R₁₁ₐ may be a deuterium atom, an unsubstituted carbazole group, a deuterium atom-substituted or unsubstituted phenyl group, an unsubstituted dibenzofuran group, an unsubstituted dibenzothiophene group, or an unsubstituted 9-phenylcarbazole group, and the others may be hydrogen atoms. “-*” may be connected to any one selected from among R₇ₐ to R₁₁ₐ, and X₁ to X₃, and n1 to n6 may be the same as defined with respect to Formula 1 above.

In an embodiment, the first compound represented by Formula 2 above may be represented by any one selected from among Formulas 2-a to 2-d below.

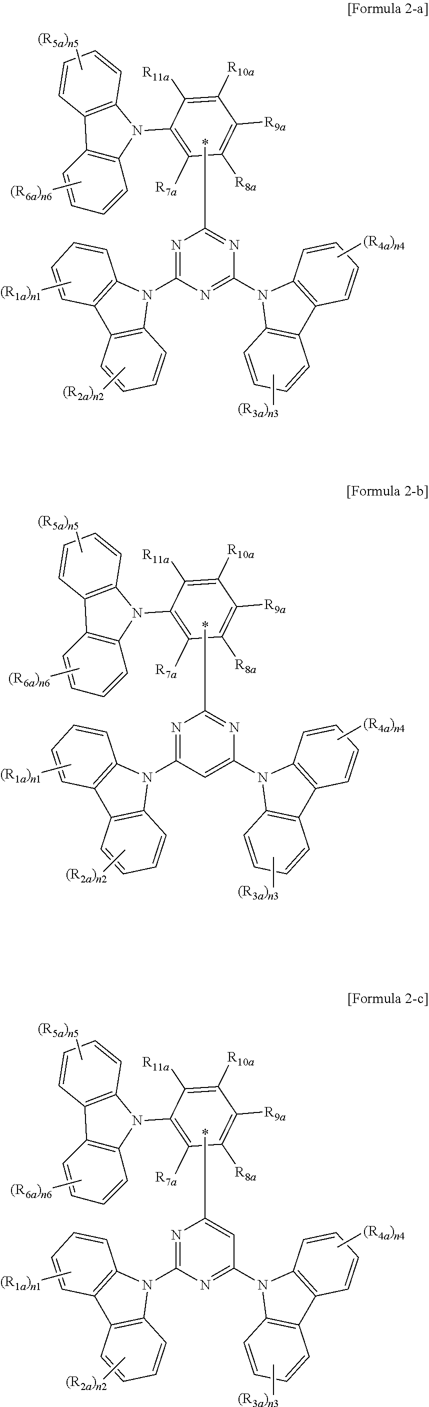

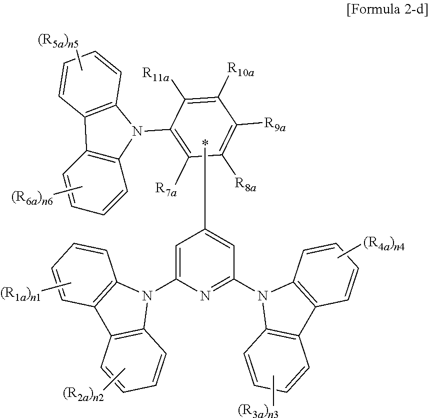

In Formulas 2-a to 2-d above, R₁ₐ to R₁₁ₐ, n1 to n6, and “-*” are the same as defined with respect to Formula 1 and Formula 2 above.

In an embodiment, the first compound represented by Formula 1 above may be represented by Formula 3-1 or Formula 3-2 below.

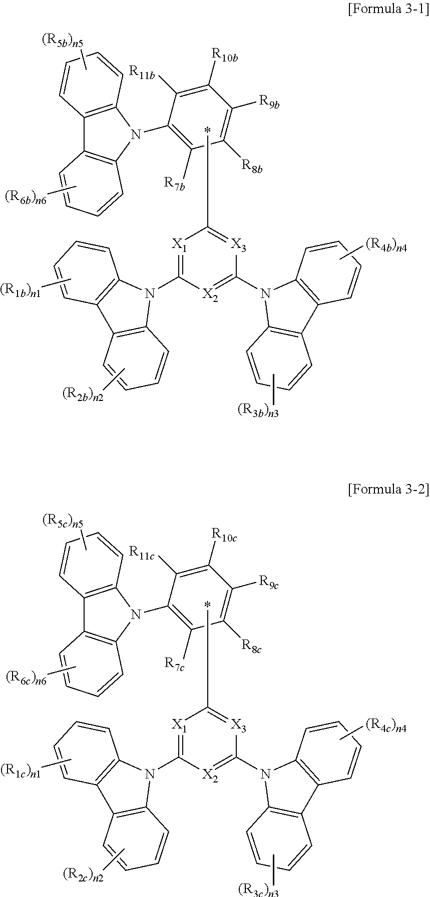

In Formula 3-1 above, at least one selected from R_{1b} to R_{11b} may be a deuterium atom, and the others may each independently be a hydrogen atom, a cyano group, a substituted or unsubstituted alkyl group having 1 to 60 carbon atoms, a substituted or unsubstituted aryl group having 6 to 60 ring-forming carbon atoms, or a substituted or unsubstituted heteroaryl group having 2 to 60 ring-forming carbon atoms, or bonded to an adjacent group to form a ring. “-*” may be connected to any one selected from among R_{7b} to R_{11b}. In Formula 3-2 above, R_{1c} to R_{6c} may each independently be a hydrogen atom, a deuterium atom, a cyano group, a substituted or unsubstituted alkyl group having 1 to 60 carbon atoms, a substituted or unsubstituted aryl group having 6 to 60 ring-forming carbon atoms, or a substituted or unsubstituted heteroaryl group having 2 to 60 ring-forming carbon atoms, or bonded to an adjacent group to form a ring. At least one selected from R_{7c} to R_{11c} may be an unsubstituted carbazole group, a deuterium atom-substituted or unsubstituted phenyl group, an unsubstituted dibenzofuran group, an unsubstituted dibenzothiophene group, or an unsubstituted 9-phenylcarbazole group, and the others may each independently be a hydrogen atom or a deuterium atom. “-*” may be connected to any one selected from among R_{7c} to R_{11c}. In Formulas 3-1 and 3-2 above, X₁ to X₃, and n1 to n6 are the same as defined with respect to Formula 1 above.

In an embodiment, the first compound represented by Formula 3-2 above may be represented by any one selected from among Formulas 3-2a to 3-2c below.

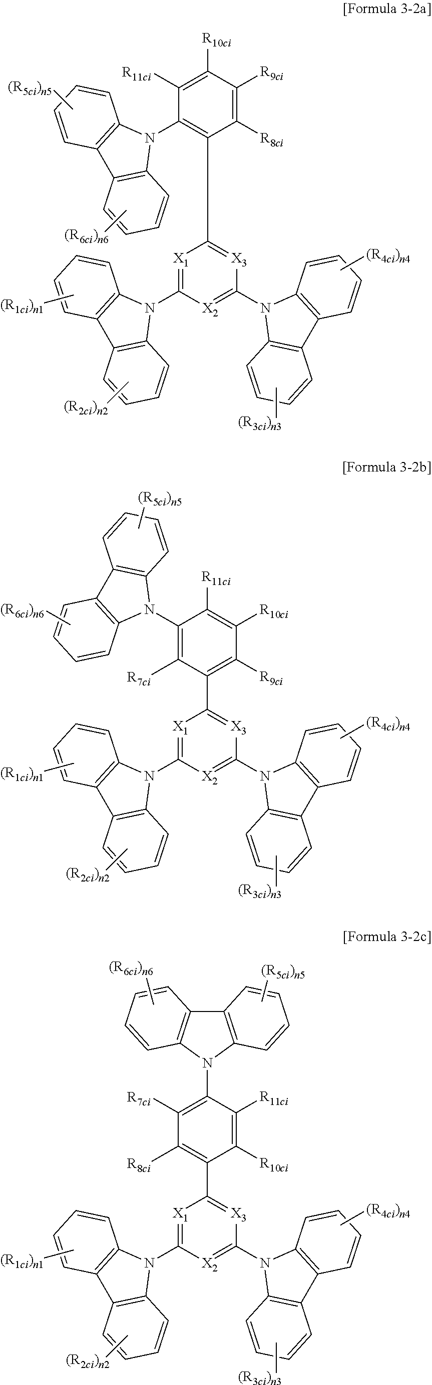

In Formula 3-2a, any one selected from among R_{8ci} to R_{11ci} may be an unsubstituted carbazole group, a deuterium atom-substituted or unsubstituted phenyl group, an unsubstituted dibenzofuran group, an unsubstituted dibenzothiophene group, or an unsubstituted 9-phenylcarbazole group, and the others may each independently be a hydrogen atom or a deuterium atom. In Formula 3-2b, any one selected from among R_{7ci}, and R_{9ci} to R_{11ci} may be an unsubstituted carbazole group, a deuterium atom-substituted or unsubstituted phenyl group, an unsubstituted dibenzofuran group, an unsubstituted dibenzothiophene group, or an unsubstituted 9-phenylcarbazole group, and the others may each independently be a hydrogen atom or a deuterium atom. In Formula 3-2c, any one selected from among R_{7ci}, R_{8ci}, R_{10ci}, and R_{11ci} may be an unsubstituted carbazole group, a deuterium atom-substituted or unsubstituted phenyl group, an unsubstituted dibenzofuran group, an unsubstituted dibenzothiophene group, or an unsubstituted 9-phenylcarbazole group, and the others may each independently be a hydrogen atom or a deuterium atom. In Formulas 3-2a to 3-2c, R_{1ci} to R_{6ci} may each independently be a hydrogen atom or a deuterium atom, and X₁ to X₃, and n1 to n6 may be the same as defined with respect to Formula 3-2 above.

In an embodiment, the first compound represented by Formula 1 above may be represented by Formula 4 below.

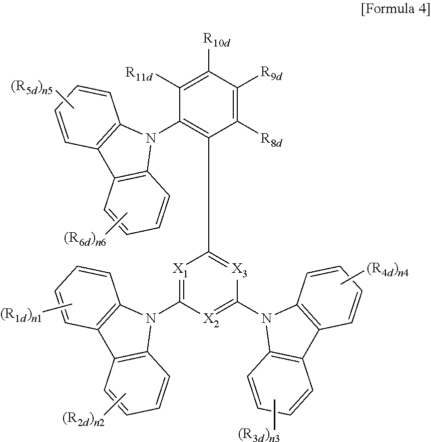

In Formulas 4 above, R_{1d} to R_{6d} may each independently be a hydrogen atom or a deuterium atom, and any one selected from among R_{8d} to R_{11d} may be a deuterium atom, an unsubstituted carbazole group, a deuterium atom-substituted or unsubstituted phenyl group, an unsubstituted dibenzofuran group, an unsubstituted dibenzothiophene group, or an unsubstituted 9-phenylcarbazole group, and the others may each independently be a hydrogen atom or a deuterium atom. X₁ to X₃, and n1 to n6 may be the same as defined with respect to Formula 1 above.

In an embodiment of the present disclosure, provided is a nitrogen-containing compound represented by Formula 1 described above.

### BRIEF DESCRIPTION OF THE DRAWINGS

The accompanying drawings are included to provide a further understanding of the subject matter of the present disclosure, and are incorporated in and constitute a part of this specification. The drawings illustrate embodiments of the present disclosure and, together with the description, serve to explain principles of the subject matter of the present disclosure. In the drawings:

FIG. 1 is a plan view showing a display device according to an embodiment;

FIG. 2 is a cross-sectional view of a display device according to an embodiment;

FIG. 3 is a cross-sectional view schematically showing a light emitting element according to an embodiment;

FIG. 4 is a cross-sectional view schematically showing a light emitting element according to an embodiment;

FIG. 5 is a cross-sectional view schematically showing a light emitting element according to an embodiment;

FIG. 6 is a cross-sectional view schematically showing a light emitting element according to an embodiment;

FIG. 7 is a cross-sectional view of a display device according to an embodiment;

FIG. 8 is a cross-sectional view of a display device according to an embodiment;

FIG. 9 is a cross-sectional view showing a display device according to an embodiment; and

FIG. 10 is a cross-sectional view showing a display device according to an embodiment.

### DETAILED DESCRIPTION

The subject matter of the present disclosure may be modified in many alternate forms, and thus, example embodiments will be illustrated in the drawings and described in more detail. It should be understood, however, that it is not intended to limit the present disclosure to the particular forms disclosed, but rather, is intended to cover all modifications, equivalents, and alternatives falling within the spirit and scope of the present disclosure.

In describing the drawings, like reference numerals are used for like elements. In the drawings, the sizes of elements may be exaggerated for clarity. It will be understood that, although the terms first, second, etc. may be used herein to describe various elements, these elements should not be limited by these terms. These terms are only used to distinguish one element from another element. For example, a first element may be referred to as a second element, and similarly, a second element may be referred to as a first element without departing from the teachings of the present disclosure. The singular forms are intended to include the plural forms as well, unless the context clearly indicates otherwise.

In the present description, it should be understood that the terms “comprise”, or “have” are intended to specify the presence of stated features, integers, steps, operations, elements, components, or combinations thereof in the disclosure, but do not preclude the presence or addition of one or more other features, integers, steps, operations, elements, components, or combinations thereof.

In the present description, it should be understood that when an element such as a layer, a film, a region, or a substrate is referred to as being “on” or “above” another element, it may be “directly on” the other element or intervening elements may also be present. In contrast, it should be understood that when an element such as a layer, a film, a region, or a substrate is referred to as being “beneath” or “under” another element, it may be “directly under” the other element or intervening elements may also be present. In addition, in the present description, it should be understood that when an element is referred to as being “on”, it may be as being “above” or “under” the other element.

In the present description, the term “substituted or unsubstituted” may indicate that one is substituted or unsubstituted with at least one substituent selected from the group consisting of a deuterium atom, a halogen atom, a cyano group, a nitro group, an amino group, a silyl group, oxy group, thio group, sulfinyl group, sulfonyl group, carbonyl group, a boron group, a phosphine oxide group, a phosphine sulfide group, an alkyl group, an alkenyl group, an alkynyl group, a hydrocarbon ring group, an aryl group, and a heterocyclic group. In addition, each of the substituents described above may be substituted or unsubstituted. For example, a biphenyl group may be interpreted as an aryl group or as a phenyl group substituted with a phenyl group.

In the present description, the term “bonded to an adjacent group to form a ring” may indicate that one is bonded to an adjacent group to form a substituted or unsubstituted hydrocarbon ring, or a substituted or unsubstituted heterocycle. The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocyclic or polycyclic. In addition, the rings formed by being bonded to each other may be connected to another ring to form a spiro structure.

As used herein, the term “an adjacent group” may refer to a substituent substituted for an atom which is directly connected to an atom substituted with a corresponding substituent, another substituent substituted for an atom which is substituted with a corresponding substituent, or a substituent sterically positioned at the nearest position to a corresponding substituent. For example, two methyl groups in 1,2-dimethylbenzene may be interpreted as mutually “adjacent groups” and two ethyl groups in 1,1-diethylcyclopentane may be interpreted as mutually “adjacent groups”. In addition, two methyl groups in 4,5-dimethylphenanthrene may be interpreted as mutually “adjacent groups”.

As used herein, examples of a halogen atom may include a fluorine atom, a chlorine atom, a bromine atom, and an iodine atom.

In the present description, an alkyl group may be a linear, branched or cyclic type (or kind). The number of carbon atoms in the alkyl group is 1 to 50, 1 to 30, 1 to 20, 1 to 10, or 1 to 6. Examples of the alkyl group may include a methyl group, an ethyl group, an n-propyl group, an isopropyl group, an n-butyl group, a s-butyl group, a t-butyl group, an i-butyl group, a 2-ethylbutyl group, a 3,3-a dimethylbutyl group, an n-pentyl group, an i-pentyl group, a neopentyl group, a t-pentyl group, a cyclopentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, a cyclohexyl group, a 4-methylcyclohexyl group, a 4-t-butylcyclohexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, a cyclooctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, and the like, but are not limited thereto.

As used herein, an alkenyl group refers to a hydrocarbon group including at least one carbon double bond in the middle or end of an alkyl group having 2 or more carbon atoms. The alkenyl group may be linear or branched. The number of carbon atoms is not particularly limited, but may be 2 to 30, 2 to 20, or 2 to 10. Examples of the alkenyl group include a vinyl group, a 1-butenyl group, a 1-pentenyl group, a 1,3-butadienyl group, a styrenyl group, a styryl vinyl group, and the like, but are not limited thereto.

As used herein, an alkynyl group refers to a hydrocarbon group including at least one carbon triple bond in the middle or end of an alkyl group having 2 or more carbon atoms. The alkynyl group may be linear or branched. The number of carbon atoms is not particularly limited, but may be 2 to 30, 2 to 20, or 2 to 10. Examples of the alkynyl group may include an ethynyl group, a propynyl group, and the like, but are not limited thereto.

As used herein, a hydrocarbon ring group refers to any suitable functional group or substituent derived from an aliphatic hydrocarbon ring. The hydrocarbon ring group may be a saturated hydrocarbon ring group having 5 to 20 ring-forming carbon atoms.

As used herein, an aryl group refers to any suitable functional group or substituent derived from an aromatic hydrocarbon ring. The aryl group may be a monocyclic aryl group or a polycyclic aryl group. The number of ring-forming carbon atoms in the aryl group may be 6 to 60, 6 to 30, 6 to 20, or 6 to 15. Examples of the aryl group may include a phenyl group, a naphthyl group, a fluorenyl group, an anthracenyl group, a phenanthryl group, a biphenyl group, a terphenyl group, a quaterphenyl group, a quinquephenyl group, a sexiphenyl group, a triphenylenyl group, a pyrenyl group, a benzofluoranthenyl group, a chrysenyl group, and the like, but are not limited thereto.

In the present description, a fluorenyl group may be substituted, and two substituents may be bonded to each other to form a spiro structure. An example that the fluorenyl group is substituted is as follows. However, the embodiment of the present disclosure is not limited thereto.

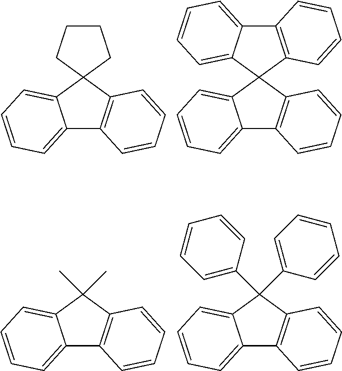

In the present description, a heterocyclic group refers to any suitable functional group or substituent derived from a ring containing at least one selected from B, O, N, P, Si, and S as a hetero atom. The heterocyclic group includes an aliphatic heterocyclic group and an aromatic heterocyclic group. The aromatic heterocyclic group may be a heteroaryl group. The aliphatic heterocycle and the aromatic heterocycle may be monocyclic or polycyclic. When the heterocyclic group contains two or more hetero atoms, the two or more hetero atoms may be the same as or different from each other.

In the present description, the aliphatic heterocyclic group may contain at least one selected from B, O, N, P, Si and S as a hetero atom. The number of ring-forming carbon atoms in the aliphatic heterocyclic group may be 2 to 30, 2 to 20, or 2 to 10. Examples of the aliphatic heterocyclic group include an oxirane group, a thiirane group, a pyrrolidine group, a piperidine group, a tetrahydrofuran group, a tetrahydrothiophene group, a thiane group, a tetrahydropyran group, a 1,4-dioxane group, and the like, but are not limited to thereto.

As used herein, a heteroaryl group may include at least one selected from B, O, N, P, Si, and S as a hetero atom. When the heteroaryl group contains two or more hetero atoms, the two or more hetero atoms may be the same as or different from each other. The heteroaryl group may be a monocyclic heterocyclic group or a polycyclic heterocyclic group. The number of ring-forming carbon atoms in the heteroaryl group may be 2 to 60, 2 to 30, 2 to 20, or 2 to 10. Examples of the heteroaryl group may include a thiophene group, a furan group, a pyrrole group, an imidazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzoimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, and the like, but are not limited thereto.

As used herein, the above description of the aryl group may be applied to an arylene group, except that the arylene group is a divalent group. The above description of the heteroaryl group may be applied to a heteroarylene group, except that the heteroarylene group is a divalent group.

In the present description, a silyl group includes an alkyl silyl group and an aryl silyl group. Examples of the silyl group include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and the like, but are not limited thereto.

In the present description, the number of carbon atoms in an amino group is not particularly limited, but may be 1 to 30. The amino group may include an alkyl amino group, an aryl amino group, or a heteroaryl amino group. Examples of the amino group include a methylamino group, a dimethylamino group, a phenylamino group, a diphenylamino group, a naphthylamino group, a 9-methyl-anthracenylamino group, a triphenylamino group, and the like, but are not limited thereto.

In the present description, the number of carbon atoms in a carbonyl group is not particularly limited, but may be 1 to 40, 1 to 30, or 1 to 20. For example, the carbonyl group may have the following structure, but is not limited thereto.

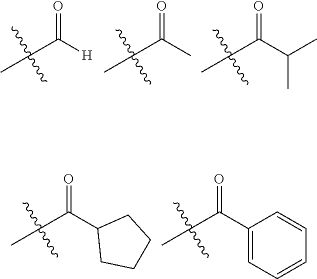

In the present description, the number of carbon atoms in a sulfinyl group and a sulfonyl group is not particularly limited, but may be 1 to 30. The sulfinyl group may include an alkyl sulfinyl group and an aryl sulfinyl group. The sulfonyl group may include an alkyl sulfonyl group and an aryl sulfonyl group.

As used herein, a thio group may include an alkyl thio group and an aryl thio group. The thio group may indicate the one that a sulfur atom is bonded to an alkyl group or an aryl group as defined above. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, and the like, but are not limited to thereto.

As used herein, an oxy group may indicate the one that an oxygen atom is bonded to an alkyl group or aryl group as defined above. The oxy group may include an alkoxy group and an aryl oxy group. The alkoxy group may be linear, branched or cyclic. The number of carbon atoms in the alkoxy group is not particularly limited, but may be, for example, 1 to 20, or 1 to 10. Examples of the oxy group include methoxy, ethoxy, n-propoxy, isopropoxy, butoxy, pentyloxy, hexyloxy, octyloxy, nonyloxy, decyloxy, benzyloxy, and the like, but are not limited thereto.

In the present description, a boron group may refer to one that a boron atom is bonded to an alkyl group or aryl group as defined above. The boron group includes an alkyl boron group and an aryl boron group. Examples of the boron group include a dimethyl boron group, a diethyl boron group, a t-butylmethyl boron group, a diphenyl boron group, a phenyl boron group, and the like, but are not limited thereto.

In the present description, the number of carbon atoms in an amine group is not particularly limited, but may be 1 to 30. The amine group may include an alkyl amine group and an aryl amine group. Examples of the amine group include a methylamine group, a dimethylamine group, a phenylamine group, a diphenylamine group, a naphthylamine group, a 9-methyl-anthracenylamine group, a triphenylamine group, and the like, but are not limited thereto.

In the present description, examples of the alkyl group include an alkylthio group, an alkyl sulfoxy group, an alkylaryl group, an alkylamino group, an alkyl boron group, an alkyl silyl group, and an alkyl amine group.

In the present description, examples of the aryl group include an aryloxy group, an arylthio group, an aryl sulfoxy group, an arylamino group, an aryl boron group, an aryl silyl group, and an aryl amine group.

As used herein, a direct linkage may refer to a single bond.

In the present description, “ [symbol] ” and “-*” indicate positions to be connected.

Hereinafter, embodiments of the present disclosure will be described with reference to the accompanying drawings.

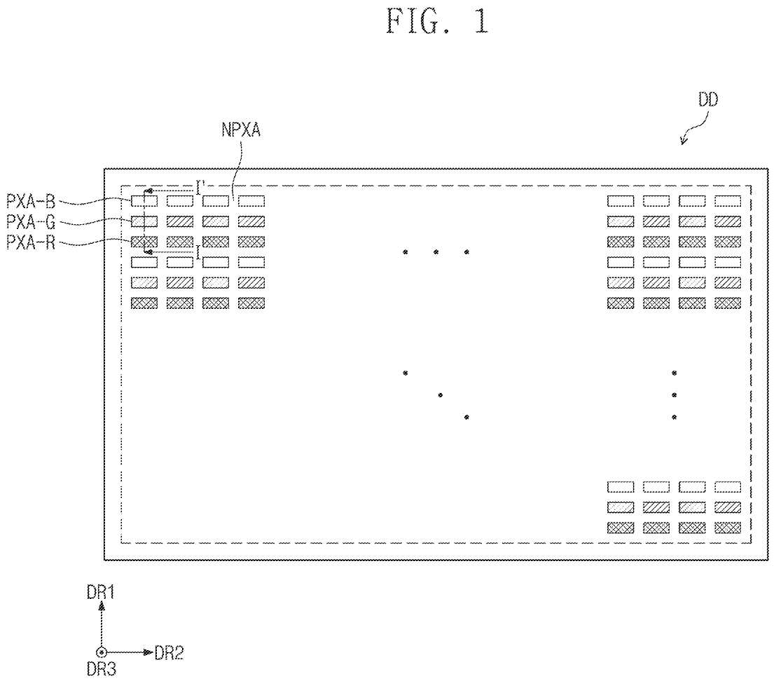

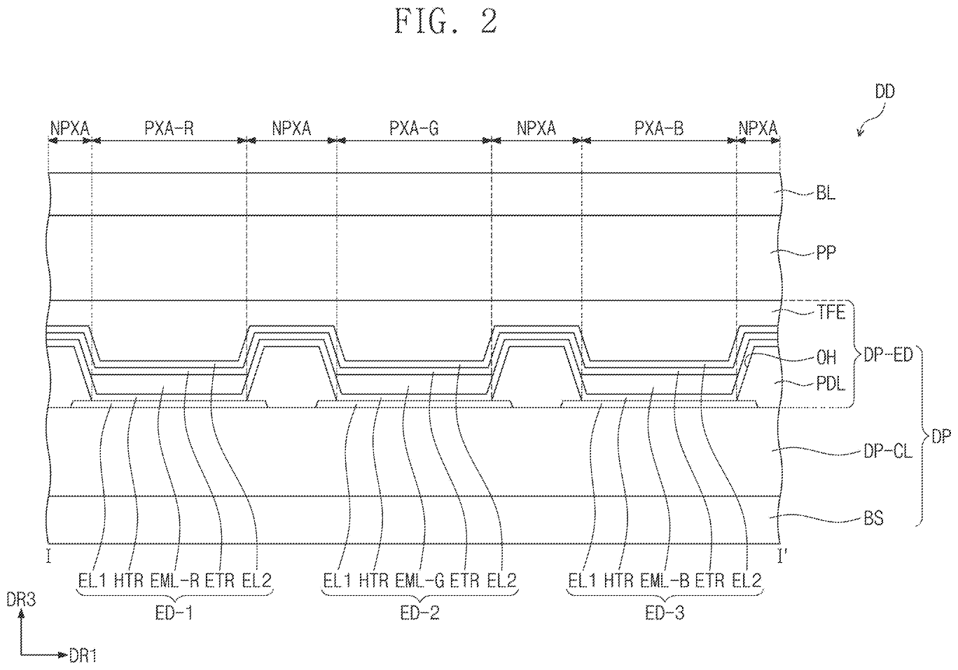

FIG. 1 is a plan view showing an embodiment of a display device DD. FIG. 2 is a cross-sectional view of a display device DD of an embodiment. FIG. 2 is a cross-sectional view showing a portion corresponding to line I-I′ of FIG. 1.

A display device DD may include a display panel DP and an optical layer PP on the display panel DP. The display panel DP includes light emitting elements ED-1, ED-2, and ED-3. The display device DD may include a plurality of light emitting elements ED-1, ED-2, and ED-3. The optical layer PP may be on the display panel DP to control reflected light in the display panel DP due to external light. The optical layer PP may include, for example, a polarizing layer and/or a color filter layer. In one or more embodiments, the optical layer PP may be omitted from the display device DD.

A base substrate BL may be on the optical layer PP. The base substrate BL may be a member providing a base surface on which the optical layer PP is provided. The base substrate BL may be a glass substrate, a metal substrate, a plastic substrate, and/or the like. However, the embodiment of the present disclosure is not limited thereto, and the base substrate BL may be an inorganic layer, an organic layer, or a composite material layer. In one or more embodiments, the base substrate BL may be omitted.

The display device DD according to an embodiment may further include a filling layer. The filling layer may be between a display element layer DP-ED and the base substrate BL. The filling layer may be an organic material layer. The filling layer may include at least one selected from among an acrylic-based resin, a silicone-based resin, and an epoxy-based resin.

The display panel DP may include a base layer BS, a circuit layer DP-CL provided on the base layer BS, and a display element layer DP-ED. The display element layer DP-ED may include pixel defining films PDL, a plurality of light emitting elements ED-1, ED-2, and ED-3 between the pixel defining films PDL, and an encapsulation layer TFE on the plurality of light emitting elements ED-1, ED-2, and ED-3.

The base layer BS may be a member providing a base surface in which the display element layer DP-ED is provided. The base layer BS may be a glass substrate, a metal substrate, a plastic substrate, and/or the like. However, the embodiment of the present disclosure is not limited thereto, and the base layer BS may be an inorganic layer, an organic layer, or a composite material layer.

In an embodiment, the circuit layer DP-CL may be on the base layer BS, and the circuit layer DP-CL may include a plurality of transistors. The transistors may each include a control electrode, an input electrode, and an output electrode. For example, the circuit layer DP-CL may include a switching transistor and a driving transistor for driving the plurality of light emitting elements ED-1, ED-2 and ED-3 of the display element layer DP-ED.

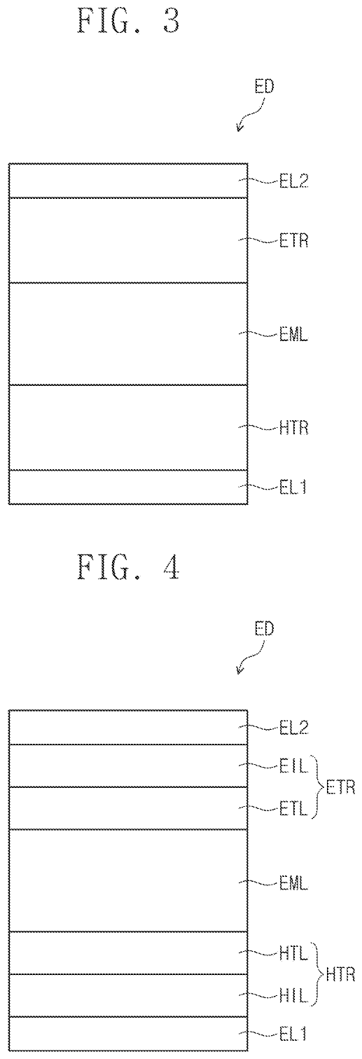

The light emitting elements ED-1, ED-2, and ED-3 may each have a structure of a light emitting element ED according to an embodiment of FIGS. 3-6, which will be further described herein below. The light emitting elements ED-1, ED-2, and ED-3 may each include a first electrode EL1, a hole transport region HTR, emission layers EML-R, EML-G, and EML-B, an electron transport region ETR, and a second electrode EL2.

FIG. 2 shows an embodiment in which the emission layers EML-R, EML-G, and EML-B of the light emitting elements ED-1, ED-2, and ED-3 are in openings OH defined in the pixel defining films PDL, and the hole transport region HTR, the electron transport region ETR, and the second electrode EL2 are provided as a common layer throughout the light emitting elements ED-1, ED-2, and ED-3. However, the embodiment of the present disclosure is not limited thereto, and unlike what is shown in FIG. 2, in an embodiment, the hole transport region HTR and the electron transport region ETR may be provided to be patterned inside the openings OH defined in the pixel defining films PDL. For example, in an embodiment, the hole transport region HTR, the emission layers EML-R, EML-G, and EML-B, and the electron transport region ETR, and the like of the light emitting elements ED-1, ED-2, and ED-3 may be patterned and provided through an inkjet printing method.

The encapsulation layer TFE may cover the light emitting elements ED-1, ED-2 and ED-3. The encapsulation layer TFE may seal the display element layer DP-ED. The encapsulation layer TFE may be a thin film encapsulation layer. The encapsulation layer TFE may be a single layer or a laminated layer of a plurality of layers. The encapsulation layer TFE includes at least one insulating layer. The encapsulation layer TFE according to an embodiment may include at least one inorganic film (hereinafter, an encapsulation inorganic film). In addition, the encapsulation layer TFE according to an embodiment may include at least one organic film (hereinafter, an encapsulation organic film) and at least one encapsulation inorganic film.

The encapsulation inorganic film protects the display element layer DP-ED from moisture/oxygen, and the encapsulation organic film protects the display element layer DP-ED from foreign substances such as dust particles. The encapsulation inorganic film may include silicon nitride, silicon oxy nitride, silicon oxide, titanium oxide, aluminum oxide, and/or the like, but is not particularly limited thereto. The encapsulation organic film may include an acrylic compound, an epoxy-based compound, and/or the like. The encapsulation organic film may include a photopolymerizable organic material, and is not particularly limited.

The encapsulation layer TFE may be on the second electrode EL2, and may fill the openings OH.

Referring to FIGS. 1-2, the display device DD may include non-light emitting regions NPXA and light emitting regions PXA-R, PXA-G, and PXA-B. The light emitting regions PXA-R, PXA-G, and PXA-B may each be a region emitting light generated from each of the light emitting elements ED-1, ED-2, and ED-3. The light emitting regions PXA-R, PXA-G, and PXA-B may be spaced apart from each other when viewed on a plane.

The light emitting regions PXA-R, PXA-G, and PXA-B may each be a region separated by the pixel defining films PDL. The non-light emitting regions NPXA may be regions between neighboring light emitting regions PXA-R, PXA-G, and PXA-B, and may correspond to the pixel defining films PDL. In one or more embodiments, as described herein, the light emitting regions PXA-R, PXA-G, and PXA-B may each correspond to a pixel. The pixel defining film PDL may separate the light emitting elements ED-1, ED-2 and ED-3. The emission layers EML-R, EML-G, and EML-B of the light emitting elements ED-1, ED-2 and ED-3 may be provided and separated in openings OH defined by the pixel defining films PDL.

The light emitting regions PXA-R, PXA-G, and PXA-B may be divided into a plurality of groups according to the color of light generated from the light emitting elements ED-1, ED-2, and ED-3. In the display device DD of an embodiment shown in FIGS. 1-2, three light emitting regions PXA-R, PXA-G, and PXA-B which emit red light, green light, and blue light, are shown as an example. For example, the display device DD of an embodiment may include a red light emitting region PXA-R, a green light emitting region PXA-G, and a blue light emitting region PXA-B, which are distinct from one another.

In the display device DD according to an embodiment, the plurality of light emitting elements ED-1, ED-2, and ED-3 may emit light having different wavelength ranges. For example, in an embodiment, the display device DD may include a first light emitting element ED-1 emitting red light, a second light emitting element ED-2 emitting green light, and a third light emitting element ED-3 emitting blue light. In one or more embodiments, the red light emitting region PXA-R, the green light emitting region PXA-G, and the blue light emitting region PXA-B of the display device DD may correspond to the first light emitting element ED-1, the second light emitting element ED-2, and the third light emitting element ED-3, respectively.

However, the embodiment of the present disclosure is not limited thereto, and the first to third light emitting elements ED-1, ED-2 and ED-3 may emit light in the same wavelength range or emit light in at least one different wavelength range. For example, the first to third light emitting elements ED-1, ED-2, and ED-3 all may emit blue light.

The light emitting regions PXA-R, PXA-G, and PXA-B in the display device DD according to an embodiment may be arranged in the form of a stripe. Referring to FIG. 1, a plurality of red light emitting regions PXA-R, a plurality of green light emitting regions PXA-G, and a plurality of blue light emitting regions PXA-B may each be arranged along a second directional axis DR2. In one or more embodiments, the red light emitting region PXA-R, the green light emitting region PXA-G, and the blue light emitting region PXA-B may be alternately arranged in turn along a first directional axis DR1.

FIGS. 1-2 show that the light emitting regions PXA-R, PXA-G, and PXA-B are all similar in size, but the embodiment of the present disclosure is not limited thereto, and the light emitting regions PXA-R, PXA-G and PXA-B may be different in size from each other according to wavelength range of emitted light. In one or more embodiments, the areas of the light emitting regions PXA-R, PXA-G, and PXA-B may refer to areas when viewed on a plane defined by the first directional axis DR1 and the second directional axis DR2.

In one or more embodiments, the arrangement of the light emitting regions PXA-R, PXA-G, and PXA-B is not limited to what is shown in FIG. 1, and the order in which the red light emitting region PXA-R, the green light emitting region PXA-G, and the blue light emitting region PXA-B are arranged comes with varied combination according to display quality characteristics required for the display device DD. For example, the light emitting regions PXA-R, PXA-G, and PXA-B may be arranged in the form of a PENTILE® arrangement structure (e.g., an RGBG matrix, RGBG structure, or RGBG matrix structure) or a diamond (Diamond Pixel®) arrangement structure. PENTILE® and Diamond Pixel® are a duly registered trademark of Samsung Display Co., Ltd.

In addition, areas of each of the light emitting regions PXA-R, PXA-G, and PXA-B may be different in size from one another. For example, in an embodiment, the green light emitting region PXA-G may be smaller than the blue light emitting region PXA-B in size, but the embodiment of the present disclosure is not limited thereto.

In the display device DD according to an embodiment, which is shown in FIG. 2, at least one selected from the first to third light emitting elements ED-1, ED-2, and ED-3 may include a nitrogen-containing compound according to an embodiment, which will be further described herein below.

Hereinafter, FIGS. 3-6 are cross-sectional views schematically showing a light emitting element according to an embodiment. The light emitting element ED according to an embodiment may include a first electrode EL1, a second electrode EL2 facing the first electrode EL1, and at least one functional layer between the first electrode EL1 and the second electrode EL2. The light emitting element ED according to an embodiment may include a nitrogen-containing compound according to an embodiment, which will be further described herein below, in at least one functional layer. In one or more embodiments, the nitrogen-containing compound according to an embodiment may be referred to as a first compound herein.

The light emitting element ED may include a hole transport region HTR, an emission layer EML, and an electron transport region ETR, which are sequentially stacked, as at least one functional layer. Referring to FIG. 3, the light emitting element ED according to an embodiment may include a first electrode EL1, a hole transport region HTR, an emission layer EML, an electron transport region ETR, and a second electrode EL2. In addition, the light emitting element ED according to an embodiment may include a nitrogen-containing compound according to an embodiment, which will be further described herein below, in the emission layer EML. However, the embodiment of the present disclosure is not limited thereto, and the light emitting element ED of an embodiment may include a nitrogen-containing compound of an embodiment in a buffer layer included in the hole transport region HTR.

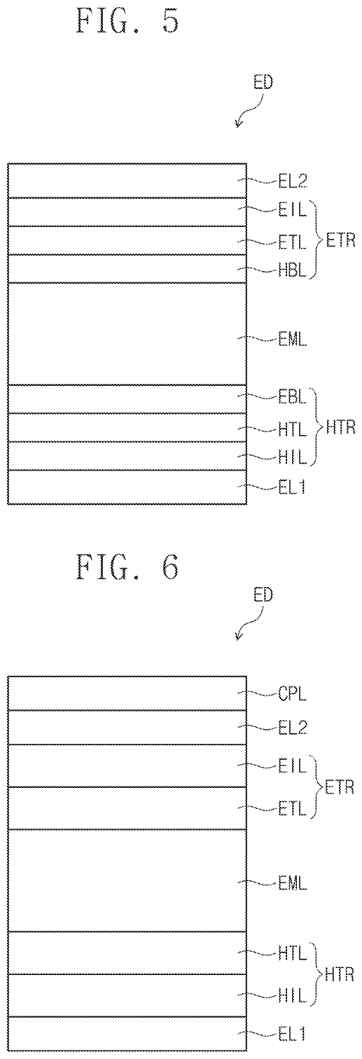

FIG. 4 shows, compared with FIG. 3, a cross-sectional view of a light emitting element ED of an embodiment in which the hole transport region HTR includes a hole injection layer HIL and a hole transport layer HTL, and the electron transport region ETR includes an electron injection layer EIL and an electron transport layer ETL. In addition, FIG. 5 shows, compared with FIG. 3, a cross-sectional view of a light emitting element ED according to an embodiment in which the hole transport region HTR includes a hole injection layer HIL, a hole transport layer HTL, and an electron blocking layer EBL, and the electron transport region ETR includes an electron injection layer EIL, an electron transport layer ETL, and a hole blocking layer HBL. FIG. 6 shows, compared with FIG. 4, a cross-sectional view of a light emitting element ED according to an embodiment, in which a capping layer CPL on the second electrode EL2 is provided.

In the light emitting element ED according to an embodiment, the first electrode EL1 has conductivity (e.g., electrical conductivity). The first electrode EL1 may be formed of a metal material, a metal alloy and/or a conductive compound (e.g., an electrically conductive compound). The first electrode EL1 may be an anode or a cathode. However, the embodiment of the present disclosure is not limited thereto. In addition, the first electrode EL1 may be a pixel electrode. The first electrode EL1 may be a transmissive electrode, a transflective electrode, or a reflective electrode. The first electrode may include at least one selected from Ag, Mg, Cu, Al, Pt, Pd, Au, Ni, Nd, Ir, Cr, Li, Ca, LiF, Mo, Ti, W, In, Sn, and Zn, two or more compounds selected therefrom, two or more mixtures selected therefrom, and/or an oxide thereof.

When the first electrode EL1 is the transmissive electrode, the first electrode EL1 may include a transparent metal oxide such as indium tin oxide (ITO), indium zinc oxide (IZO), zinc oxide (ZnO), and/or indium tin zinc oxide (ITZO). When the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca (a stack structure of LiF and Ca), LiF/Al (a stack structure of LiF and Al), Mo, Ti, W, a compound thereof, and/or a mixture thereof (e.g., a mixture of Ag and Mg). In one or more embodiments, the first electrode EL1 may have a multilayer structure including a reflective film or a transflective film formed of the above-described materials, and a transparent conductive film formed of indium tin oxide (ITO), indium zinc oxide (IZO), zinc oxide (ZnO), indium tin zinc oxide (ITZO), and/or the like. For example, the first electrode EL1 may have a three-layer structure of ITO/Ag/ITO, but is not limited thereto. In addition, the embodiment of the present disclosure is not limited thereto, and the first electrode EL1 may include the above-described metal materials, a combination of two or more metal materials selected from the above-described metal materials, and/or oxides of the above-described metal materials. The first electrode EL1 may have a thickness of about 700 Å to about 10000 Å. For example, the first electrode EL1 may have a thickness of 1000 Å to about 3000 Å.

The hole transport region HTR is provided on the first electrode EL1. The hole transport region HTR may have a single layer formed of a single material, a single layer formed of a plurality of different materials, or a multilayer structure having a plurality of layers formed of a plurality of different materials.

The hole transport region HTR may include at least one selected from among a hole injection layer HIL, a hole transport layer HTL, a buffer layer, a light emitting auxiliary layer, and an electron blocking layer EBL. In one or more embodiments, the hole transport region HTR may include a plurality of hole transport layers that are stacked.

In addition, alternatively, the hole transport region HTR may have a single-layer structure formed of the hole injection layer HIL or the hole transport layer HTL, or a single-layer structure formed of a hole injection material or a hole transport material. In an embodiment, the hole transport region HTR may have a single-layer structure formed of a plurality of different materials, or a structure in which a hole injection layer HIL/hole transport layer HTL, a hole injection layer HIL/hole transport layer HTL/buffer layer, a hole injection layer HIL/buffer layer, or a hole transport layer HTL/buffer layer are stacked in order from the first electrode EL1, but the embodiment of the present disclosure is not limited thereto.

The hole transport region HTR may have, for example, a thickness of about 50 Å to about 15000 Å. The hole transport region HTR may be formed using various suitable methods such as a vacuum deposition method, a spin coating method, a cast method, a Langmuir-Blodgett (LB) method, an inkjet printing method, a laser printing method, and/or a laser induced thermal imaging (LITI) method.

In the light emitting element ED according to an embodiment, the hole transport region HTR may include a compound represented by Formula H-1 below.

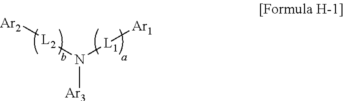

In Formula H-1 above, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms. A and b may each independently be an integer of 0 to 10. In one or more embodiments, when a or b is an integer of 2 or greater, a plurality of L₁'s and L₂'s may each independently be a substituted or unsubstituted arylene group having 6 to 30 ring-forming carbon atoms, or a substituted or unsubstituted heteroarylene group having 2 to 30 ring-forming carbon atoms.

In Formula H-1, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. In addition, in Formula H-1, Ar₃ may be a substituted or unsubstituted aryl group having 6 to 30 ring-forming carbon atoms.

A compound represented by Formula H-1 above may be a monoamine compound. In one or more embodiments, the compound represented by Formula H-1 may be a diamine compound in which at least one selected from Ar₁ to Ar₃ includes an amine group as a substituent. In addition, the compound represented by Formula H-1 may be a carbazole-based compound including a substituted or unsubstituted carbazole group in at least one selected from Ar₁ and Ar₂ or a substituted or unsubstituted fluorene-based group in at least one selected from Ar₁ and Ar₂.

The compound represented by Formula H-1 may be represented by any one selected from among compounds from Compound Group H below. However, the compounds listed in Compound Group H below are presented as an example, and the compound represented by Formula H-1 is not limited to the those listed in Compound Group H below.

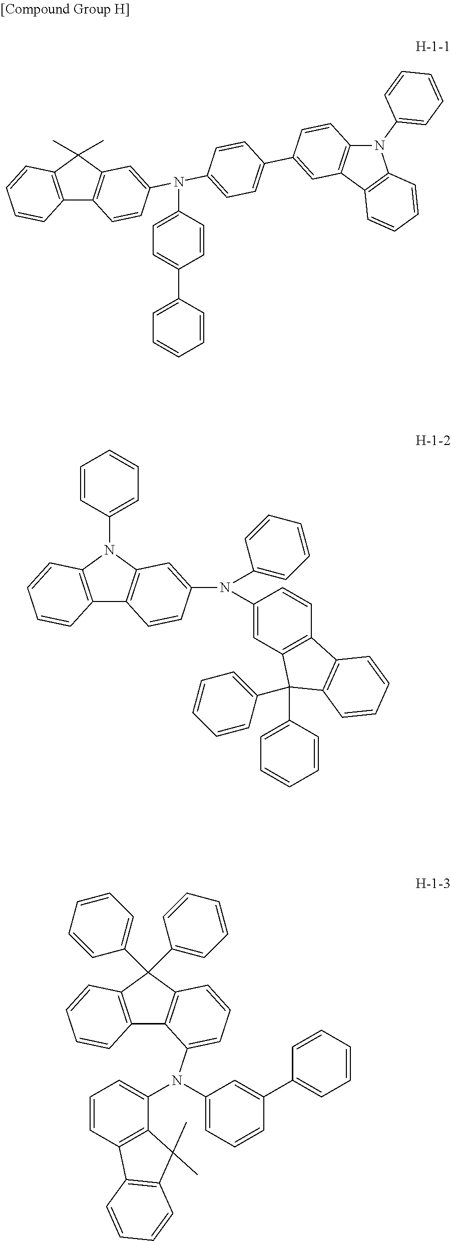 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21]

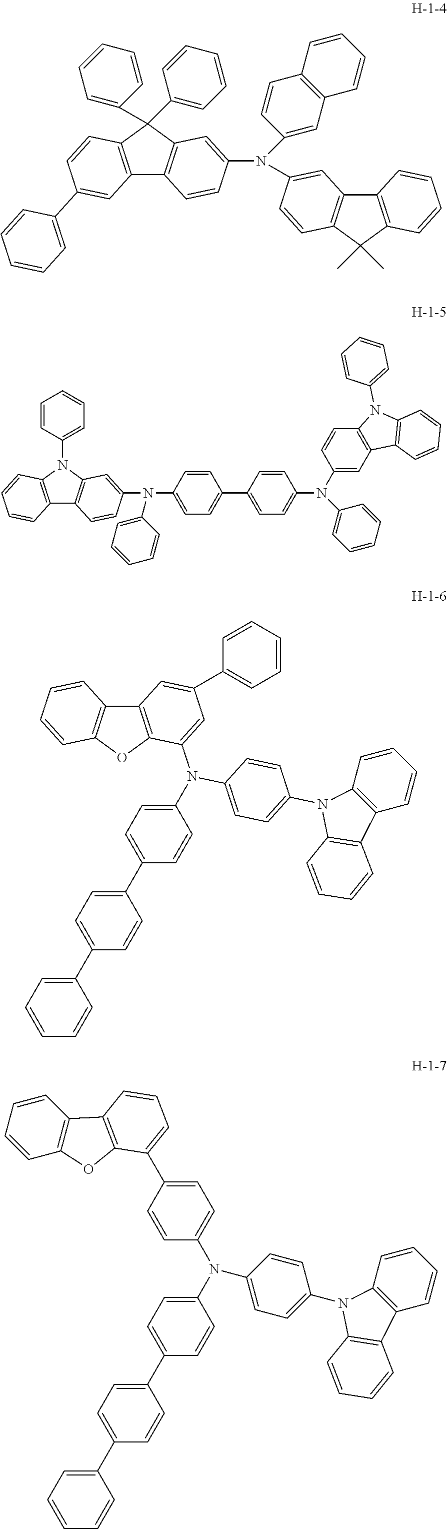 [SMILES: CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21 ; c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

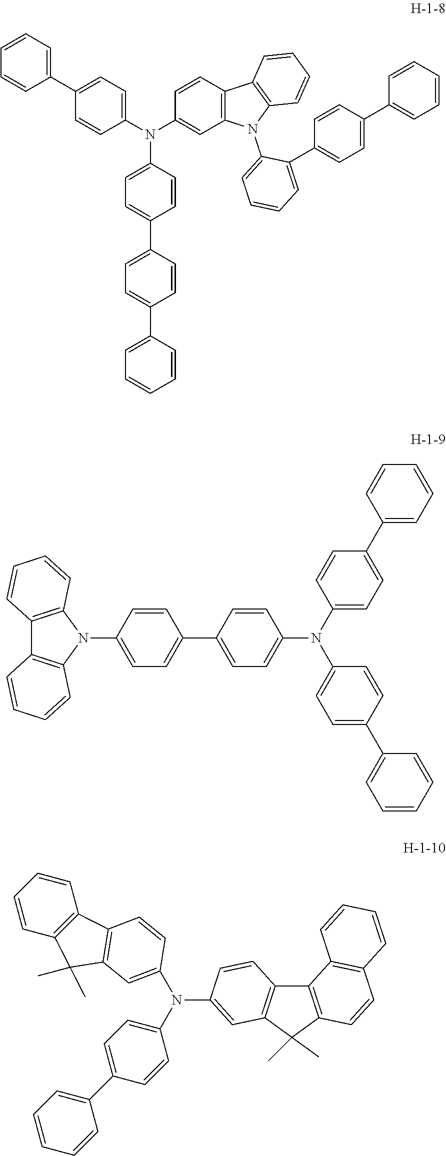 [SMILES: c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2ccccc2-c2ccc(N(c3ccc(-c4ccccc4)cc3)c3ccc4c(c3)C(C)(C)c3ccc5ccccc5c3-4)cc21]

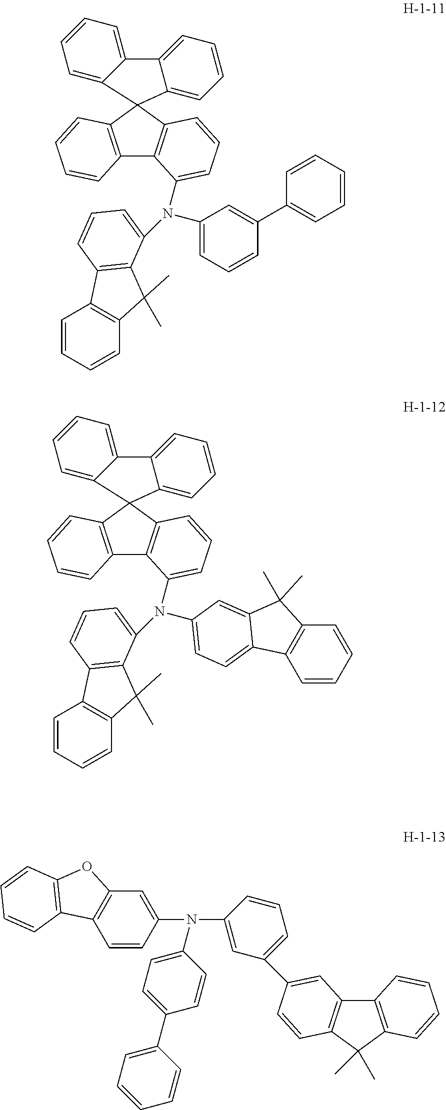 [SMILES: CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21]

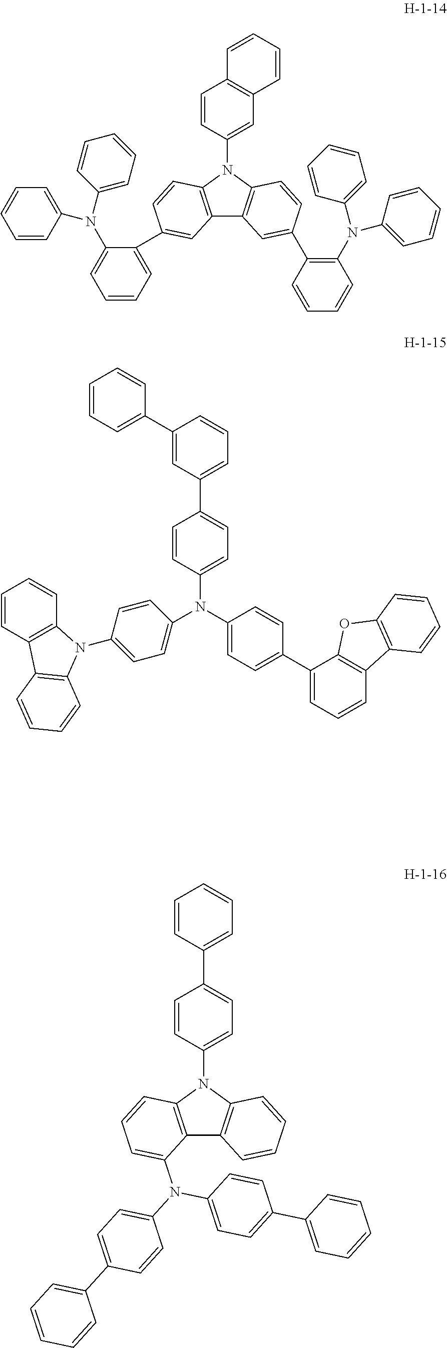 [SMILES: c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1]

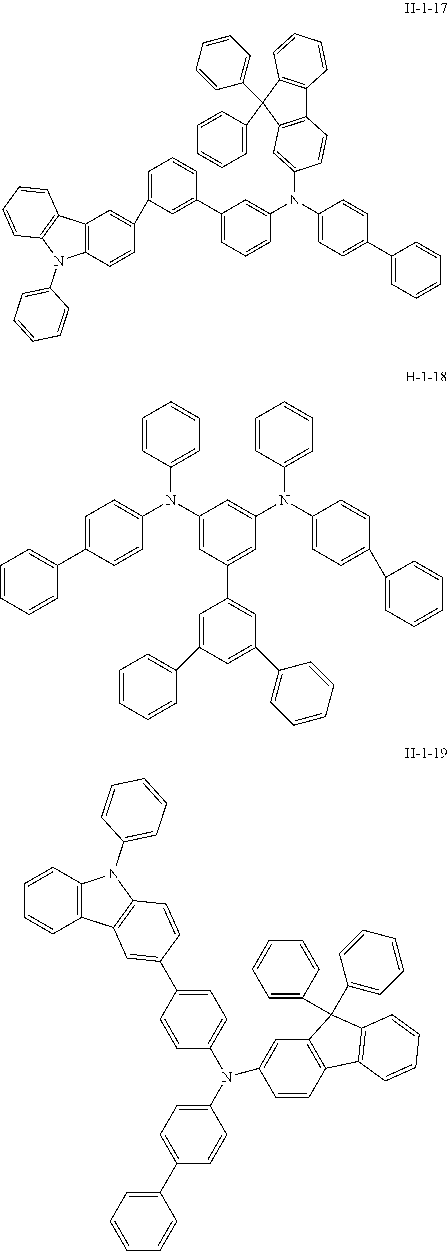 [SMILES: c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

The hole transport region HTR may further include a phthalocyanine compound such as copper phthalocyanine, N¹,N¹′-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino] triphenylamine (m-MTDATA), 4,4′,4″-tris(N,N-diphenylamino)triphenylamine (TDATA), 4,4′,4″-tris[N(1-naphthyl)-N-phenylamino]-triphenylamine (1-TNATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalene-I-yl)-N,N′-diphenyl-benzidine (NPB or NPD, a-NPD), triphenylamine-containing polyetherketone (TPAPEK), 4-isopropyl-4′-methyldiphenyliodonium [tetrakis(pentafluorophenyl)borate], dipyrazino[2,3-f: 2′,3′-h]quinoxaline-2,3,6,7,10,11-hexacarbonitrile (HAT-CN), and/or the like.

The hole transport region HTR may include carbazole-based derivatives such as N-phenyl carbazole and polyvinyl carbazole, fluorene-based derivatives, triphenylamine-based derivatives such as 4,4′,4″-tris(N-carbazolyl)triphenylamine (TCTA), N, N′-bis(3-methylphenyl)-N, N′-diphenyl-[1,1′-biphenyl]-4,4′-diamine (TPD), N,N′-di(naphthalene-1-yl)-N,N′-diphenyl-benzidine (NPB), 4,4′-cyclohexylidene bis[N,N-bis(4-methylphenyl)benzenamine] (TAPC), 4,4′-bis[N,N′-(3-tolyl)amino]-3,3′-dimethylbiphenyl (HMTPD), 1,3-bis(N-carbazolyl)benzene (mCP), and/or the like.

In addition, the hole transport region HTR may include 9-(4-tert-Butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), and/or the like.

The hole transport region HTR may include the compounds of the hole transport region HTR described above in at least one selected from among the hole injection layer HIL, the hole transport layer HTL, and the electron blocking layer EBL.

The hole transport region HTR may have a thickness of about 100 Å to about 10000 Å, for example, about 100 Å to about 5000 Å. When the hole transport region HTR includes the hole injection layer HIL, the hole injection layer HIL may have a thickness of, for example, about 30 Å to about 1000 Å. When the hole transport region HTR includes the hole transport layer HTL, the hole transport layer HTL may have a thickness of about 30 Å to about 1000 Å. When the hole transport region HTR includes the electron blocking layer EBL, the electron blocking layer EBL may have a thickness of, for example, about 10 Å to about 1000 Å. When the thicknesses of the hole transport region HTR, the hole injection layer HIL, the hole transport layer HTL, and the electron blocking layer EBL satisfy the above-described ranges, suitable or satisfactory hole transport properties may be obtained without a substantial increase in driving voltage.

The hole transport region HTR may further include, in addition to the above-described materials, a charge generation material to increase conductivity (e.g., electrical conductivity). The charge generation material may be uniformly or non-uniformly dispersed in the hole transport region HTR. The charge generation material may be, for example, a p-dopant. The p-dopant may include at least one selected from halogenated metal compounds, quinone derivatives, metal oxides, and cyano group-containing compounds, but is not limited thereto. For example, the p-dopant may include halogenated metal compounds such as CuI and/or RbI, quinone derivatives such as tetracyanoquinodimethane (TCNQ) and/or 2,3,5,6-tetrafluoro-7,7,8,8-tetracyanoquinodimethane (F4-TCNQ), metal oxides such as tungsten oxides and/or molybdenum oxides, cyano group-containing compounds such as dipyrazino[2,3-f: 2′,3′-h] quinoxaline-2,3,6,7,10,11-hexacarbonitrile (HATCN) and/or 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), and/or the like, but is not limited thereto.

As described above, the hole transport region HTR may further include at least one selected from a buffer layer, a light emitting auxiliary layer, and an electron blocking layer EBL in addition to the hole injection layer HIL and the hole transport layer HTL. The buffer layer may compensate a resonance distance according to wavelengths of light emitted from an emission layer EML, and may thus increase light emitting efficiency. Materials which may be included in the hole transport region HTR may be used as materials included in the buffer layer. The electron blocking layer EBL is a layer that serves to prevent or reduce injection of electrons from the electron transport region ETR to the hole transport region HTR. The light emitting auxiliary layer may improve charge balance between holes and electrons. When the hole transport region HTR includes the electron blocking layer EBL, the electron blocking layer EBL may function as a light emitting auxiliary layer.

The emission layer EML is provided on the hole transport region HTR. The emission layer EML may have, for example, a thickness of about 100 Å to about 1000 Å or about 100 Å to about 300 Å. The emission layer EML may have a single layer formed of a single material, a single layer formed of a plurality of different materials, or a multilayer structure having a plurality of layers formed of a plurality of different materials.

In the light emitting element ED according to an embodiment, the emission layer EML may include a first compound. The first compound corresponds to a nitrogen-containing compound according to an embodiment. The nitrogen-containing compound of an embodiment may include a core portion of a hexagonal monocyclic ring including at least one nitrogen atom (N) as a ring-forming atom. In the nitrogen-containing compound of an embodiment, the hexagonal monocyclic ring may be a pyridine group, a pyrimidine group, or a triazine group. The nitrogen-containing compound of an embodiment may be one in which first to third substituents are directly or indirectly bonded to the nitrogen-containing core portion. The first to third substituents may be carbazole groups. In the nitrogen-containing compound of an embodiment, the first and second substituents may be directly bonded to the core portion, and the third substituent may be indirectly bonded through a linker of a benzene ring.

In the nitrogen-containing compound of an embodiment, a fourth substituent may be bonded to at least one selected from the first to third substituents and the linker. In the nitrogen-containing compound of an embodiment, three carbazole groups, which are electron donating substituents, are connected to the core portion, and the fourth substituent is additionally introduced, thereby contributing to reducing driving voltage and improving color purity of a light emitting element.

The nitrogen-containing compound according to an embodiment may be represented by Formula 1 below.

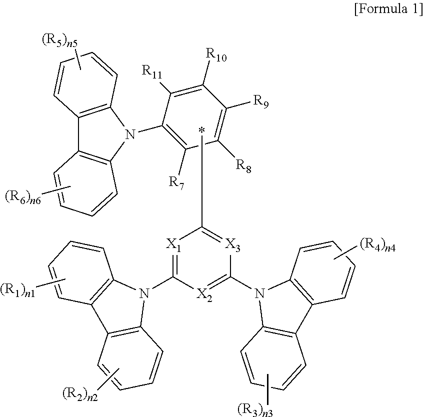

In Formula 1, at least one selected from X₁ to X₃ may be a nitrogen atom (N), and the others may each independently be CRₓ. For example, X₁ to X₃ may all be N. When X₁ to X₃ are all N in Formula 1, the nitrogen-containing compound of an embodiment may include a core portion of a triazine skeleton. In addition, two of X₁ to X₃ may be N and the other one may be CRₓ. In this case, the nitrogen-containing compound of an embodiment may include a core portion of a pyrimidine skeleton. In addition, any one selected from among X₁ to X₃ may be N and the others may each independently be CRₓ. In this case, the core portion in the nitrogen-containing compound of an embodiment may include a pyridine skeleton.

In an embodiment, Rₓ may be a hydrogen atom, a deuterium atom, a substituted or unsubstituted alkyl group having 1 to 60 carbon atoms, a substituted or unsubstituted aryl group having 6 to 60 ring-forming carbon atoms, or a substituted or unsubstituted heteroaryl group having 2 to 60 ring-forming carbon atoms. For example, Rₓ may be a hydrogen atom or a deuterium atom.

In Formula 1 above, at least one selected from R₁ to R₁₁ may be a fourth substituent, and the others may each independently be a hydrogen atom, a cyano group, a substituted or unsubstituted alkyl group having 1 to 60 carbon atoms, a substituted or unsubstituted aryl group having 6 to 60 ring-forming carbon atoms, or a substituted or unsubstituted heteroaryl group having 2 to 60 ring-forming carbon atoms, or bonded to an adjacent group to form a ring. For example, at least one selected from R₁ to R₁₁ may be a fourth substituent, and the others may be hydrogen atoms. In an embodiment, when two or more of R₁ to R₁₁ are fourth substituents, the two or more fourth substituents may be the same or different. In one or more embodiments, in Formula 1, “-*” may be a position connected to any one selected from among R₇ to R₁₁. In one or more embodiments, any one selected from R₇ to R₁₁ may be “-*” or may be replaced with “-*”.

In an embodiment, the fourth substituent may be a deuterium atom, an unsubstituted carbazole group, a deuterium atom-substituted or unsubstituted phenyl group, an unsubstituted dibenzofuran group, an unsubstituted dibenzothiophene group, or an unsubstituted 9-phenylcarbazole group. In one or more embodiments, a case where X₁ to X₃ are all N, “-*” is connected to R₇, R₈ is an unsubstituted carbazole group, and R₁ to R₆ and R₉ to R₁₁ are all hydrogen atoms may be excluded.

For example, the fourth substituent may be represented by any one selected from Substituent Group 1 below. In Substituent Group 1 below, “D” is a deuterium atom, and “-*” is a position connected to Formula 1.

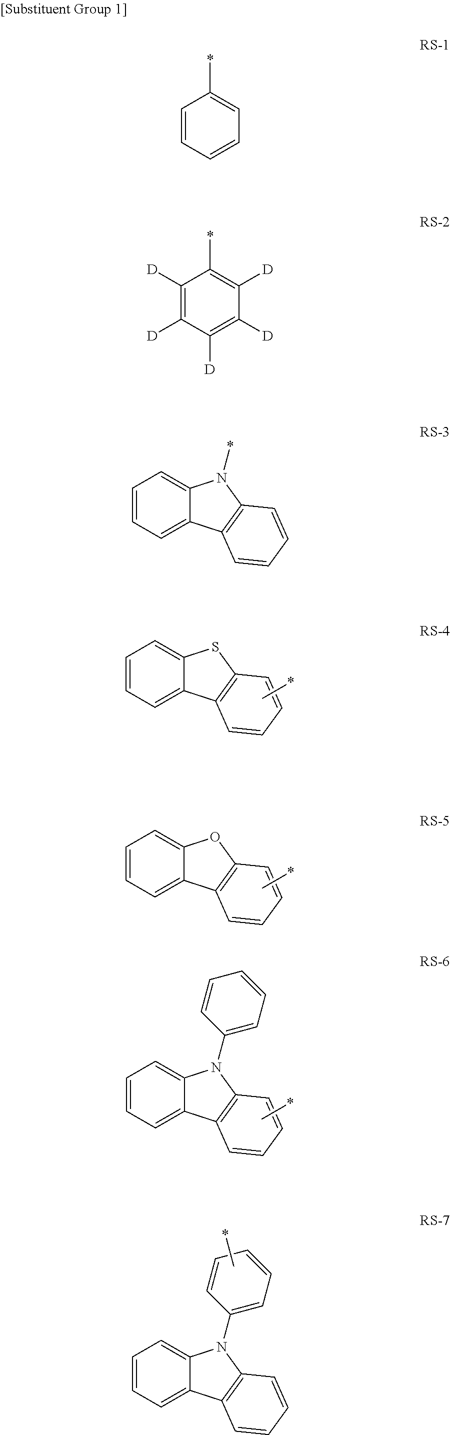

In Formula 1, n1 to n6 may each independently be an integer of 0 to 4. When n1 to n6 are 2 or greater, a plurality of R₁′ to R₆′ may each be the same or at least one may be different. In an embodiment, a case where n1 is 0 may be the same as a case where R₁ is 4 and R₁ is a hydrogen atom. A case where n1 to n6 are each 0 may be the same as a case where R₁ to R₆ are each 4 and R₁ to R₆ are all hydrogen atoms. When n1 to n6 are each 0, the nitrogen-containing compound of an embodiment may not be one that is not substituted with each of R₁ to R₆.

The nitrogen-containing compound of an embodiment may include a deuterium atom as a substituent. In an embodiment, at least one selected from X₁ to X₃ and R₁ to R₁₁ may include a deuterium atom or a substituent containing a deuterium atom. For example, in the nitrogen-containing compound represented by Formula 1, at least one selected from R₁ to R₁₁ may be a deuterium atom or a substituent containing a deuterium atom. However, this is presented as an example, and the embodiment of the present disclosure is not limited thereto.

In an embodiment, Formula 1 may be represented by Formula 2 below. Formula 2 specifies R₁ to R₁₁ in Formula 1. In Formulas 2, the same descriptions as those described above with respect to Formula 1 may be applied to X₁ to X₃ and n1 to n6.

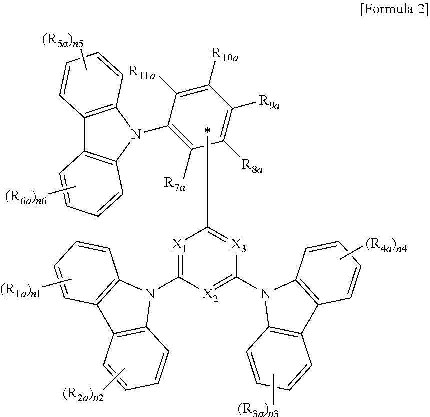

In Formula 2, at least one selected from R₁ₐ to R₁₁ₐ may be a deuterium atom, an unsubstituted carbazole group, a deuterium atom-substituted or unsubstituted phenyl group, an unsubstituted dibenzofuran group, an unsubstituted dibenzothiophene group, or an unsubstituted 9-phenylcarbazole group, and the others may be hydrogen atoms. In one or more embodiments, at least one selected from R₁ₐ to R₁₁ₐ is the fourth substituent, and the others of R₁ₐ to R₁₁ₐ other than the fourth substituent may be a hydrogen atom. In one or more embodiments, “-*” may be a position connected to any one selected from among R₇ₐ to R₁₁ₐ. In one or more embodiments, any one selected from among R₇ₐ to R₁₁ₐ may be “-*” or may be replaced with “-*”.

The nitrogen-containing compound of an embodiment may be represented by any one selected from among Formulas 2-a to 2-d below. Each of the Formulas 2-a to 2-d below represents specifies the core portion in Formula 2. Formula 2-a corresponds to a case where X₁ to X₃ in Formula 2 are all nitrogen atoms (N), and in each of Formula 2-b and Formula 2-c corresponds to a case where two of X₁ to X₃ are nitrogen atoms (N) and the other one is CRₓ in Formula 2. Formula 2-d corresponds to a case where X₂ is a nitrogen atom (N) and X₁ and X₃ are CRₓ in Formula 2. In Formulas 2-b to 2-d, Rₓ is a hydrogen atom.

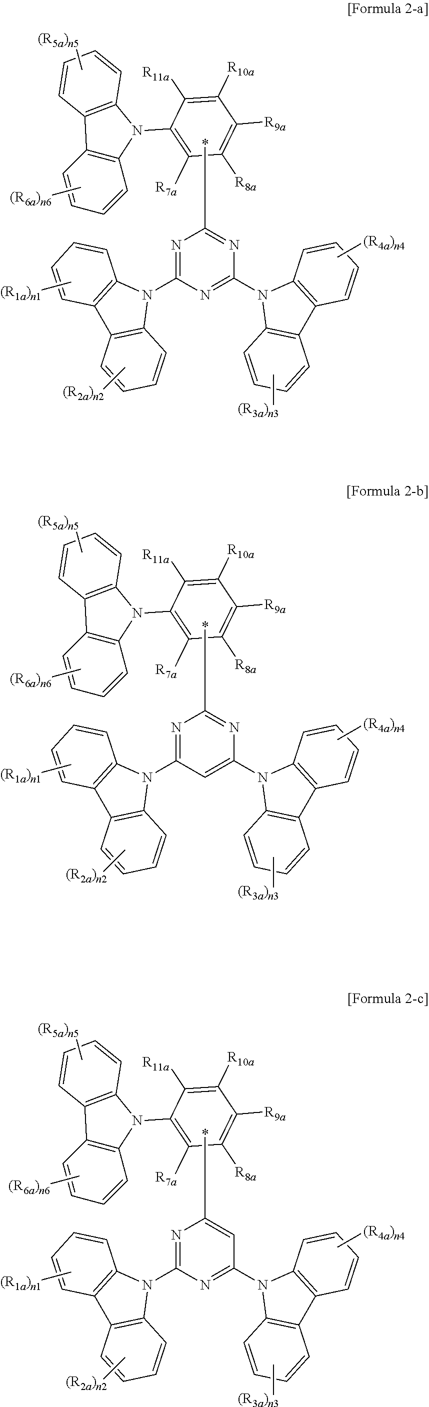

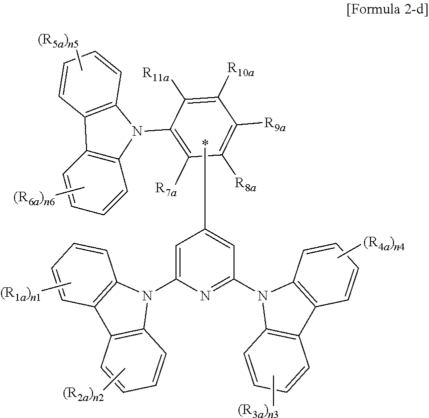

In Formulas 2-a to 2-d, the same descriptions as those described above with respect to Formulas 1 and 2 above may be applied to R₁ₐ to R₁₁ₐ, n1 to n6, and “-*”. In one or more embodiments, any one selected from R₁ₐ to R₁₁ₐ may be “-*” or may be replaced with “-*”.

The nitrogen-containing compound of an embodiment may be represented by Formula 3-1 or Formula 3-2 below. Formula 3-1 and Formula 3-2 correspond to a case where in the nitrogen-containing compound represented by Formula 1, a bonding relationship according to type (or kind) of the fourth substituent is specified. In Formulas 3-1 and 3-2 below, the same descriptions as those described above with respect to Formula 1 above may be applied to X₁ to X₃ and n1 to n6.

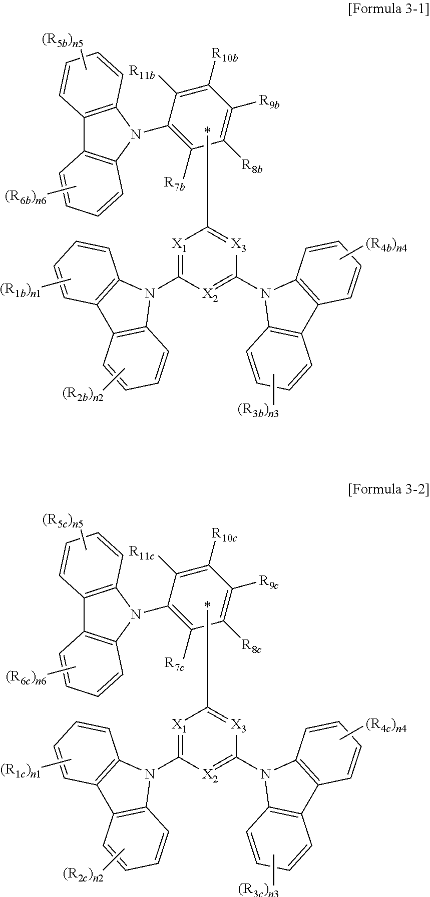

In Formula 3-1 above, at least one selected from R_{1b} to R_{11b} may be a deuterium atom, and the others may each independently be a hydrogen atom, a cyano group, a substituted or unsubstituted alkyl group having 1 to 60 carbon atoms, a substituted or unsubstituted aryl group having 6 to 60 ring-forming carbon atoms, or a substituted or unsubstituted heteroaryl group having 2 to 60 ring-forming carbon atoms, or bonded to an adjacent group to form a ring. In one or more embodiments, the nitrogen-containing compound of an embodiment represented by Formula 3-1 may include at least one deuterium atom as the fourth substituent. In an embodiment, at least one selected from R_{1b} to R₁₁ may be a deuterium atom, and the others may be hydrogen atoms. In one or more embodiments, “-*” may be connected to any one position selected from among R_{7b} to R_{11b}. In one or more embodiments, any one selected from R_{7b} to R_{11b} may be “-*” or may be replaced with “-*”. For example, at least one selected from R_{1b} to R_{6b}, and at least one of the others except for positions bonded to the core portion selected from among R_{7b} to R_{11b} may be deuterium atoms and the others, which are not deuterium atoms, selected from among R_{1b} to R_{11b} may be hydrogen atoms. In addition, at least one selected from R_{1b} to R_{6b}, or at least one of the others except for positions bonded to the core portion among R_{7b} to R_{11b} may be deuterium atoms and the others, which are not deuterium atoms, among R_{1b} to R_{11b} may be hydrogen atoms.

In Formula 3-2, R_{1c} to R_{6c} may each independently be a hydrogen atom, a deuterium atom, a cyano group, a substituted or unsubstituted alkyl group having 1 to 60 carbon atoms, a substituted or unsubstituted aryl group having 6 to 60 ring-forming carbon atoms, or a substituted or unsubstituted heteroaryl group having 2 to 60 ring-forming carbon atoms, or bonded to an adjacent group to form a ring. For example, R_{1c} to R_{6c} may each independently be a hydrogen atom or a deuterium atom.

In Formulas 3-2, at least one selected from R_{7c} to R_{11c} may be an unsubstituted carbazole group, a deuterium atom-substituted or unsubstituted phenyl group, an unsubstituted dibenzofuran group, an unsubstituted dibenzothiophene group, or an unsubstituted 9-phenylcarbazole group, and the others may each independently be a hydrogen atom or a deuterium atom. In one or more embodiments, “-*” may be connected to any one position selected from among R_{7c} to R_{11c}, or any one selected from among R_{7c} to R_{11c} may be “-*” or may be replaced with “-*”. For example, any one of the others that are not connected to the core portion selected from among R_{7c} to R_{11c} may be an unsubstituted carbazole group, a deuterium atom-substituted or unsubstituted phenyl group, an unsubstituted dibenzofuran group, an unsubstituted dibenzothiophene group, or an unsubstituted 9-phenylcarbazole group, and the others may each independently be a hydrogen atom or a deuterium atom.

In an embodiment, the nitrogen-containing compound represented by Formula 3-2 may be represented by any one selected from among Formulas 3-2a to 3-2c below. Formula 3-2a shows, as an example, a structure in which the third substituent and the nitrogen-containing core portion which are connected through a linker in Formula 3-2 are bonded in an ortho relationship. Formula 3-2b and Formula 3-2c each shows, as an example, a structure in which the third substituent and the nitrogen-containing core portion which are connected through a linker in Formula 3-2 are bonded in meta and para relationships. In Formulas 3-2a to 3-2c, X₁ to X₃ and n1 to n6 are the same as defined in Formula 3-2 above. In one or more embodiments, in Formulas 3-2a to 3-2c, the same descriptions as those described above with respect to Formula 1 may be applied to X₁ to X₃ and n1 to n6.

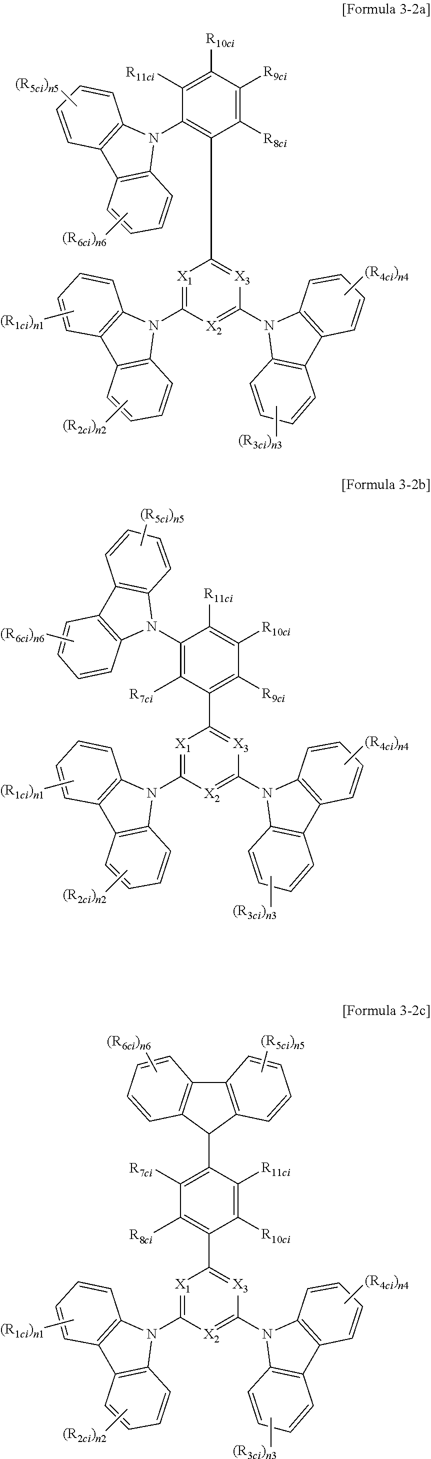

In Formula 3-2a, R_{1ci} to R_{6ci} may each independently be a hydrogen atom or a deuterium atom. Any one selected from among R_{8ci} to R_{11ci} may be an unsubstituted carbazole group, a deuterium atom-substituted or unsubstituted phenyl group, an unsubstituted dibenzofuran group, an unsubstituted dibenzothiophene group, or an unsubstituted 9-phenylcarbazole group, and the others may each independently be a hydrogen atom or a deuterium atom.

In Formula 3-2b, R_{1ci} to R_{6ci} may each independently be a hydrogen atom or a deuterium atom. Any one selected from among R_{7ci} and R_{9ci} to R_{11ci} may be an unsubstituted carbazole group, a deuterium atom-substituted or unsubstituted phenyl group, an unsubstituted dibenzofuran group, an unsubstituted dibenzothiophene group, or an unsubstituted 9-phenylcarbazole group, and the others may each independently be a hydrogen atom or a deuterium atom.

In Formula 3-c, R_{1ci} to R_{6ci} may each independently be a hydrogen atom or a deuterium atom. Any one selected from among R_{7ci}, R_{8ci}, R_{10ci}, and R_{11ci} may be an unsubstituted carbazole group, a deuterium atom-substituted or unsubstituted phenyl group, an unsubstituted dibenzofuran group, an unsubstituted dibenzothiophene group, or an unsubstituted 9-phenylcarbazole group, and the others may each independently be a hydrogen atom or a deuterium atom.

In an embodiment, the nitrogen-containing compound represented by Formula 1 may be represented by Formula 4 below. The nitrogen-containing compound of an embodiment represented by Formula 4 is one in which the bonding relationship between the third substituent and the core portion in Formula 1 is specified. In Formulas 4, the same descriptions as those described above with respect to Formula 1 may be applied to X₁ to X₃ and n1 to n6.

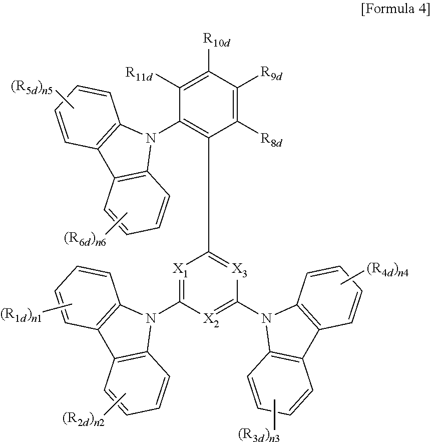

In Formula 4, R_{1ci} to R_{6d} may each independently be a hydrogen atom or a deuterium atom. Any one selected from among R_{8d} to R_{11d} may be a deuterium atom, an unsubstituted carbazole group, a deuterium atom-substituted or unsubstituted phenyl group, an unsubstituted dibenzofuran group, an unsubstituted dibenzothiophene group, or an unsubstituted 9-phenylcarbazole group, and the others may each independently be a hydrogen atom or a deuterium atom.

The nitrogen-containing compound of an embodiment may be represented by any one selected from among compounds of Compound Group 1 below. A light emitting element ED of an embodiment may include at least one selected from the compounds from Compound Group 1 below. In Compound Group 1 below, D is a deuterium atom.

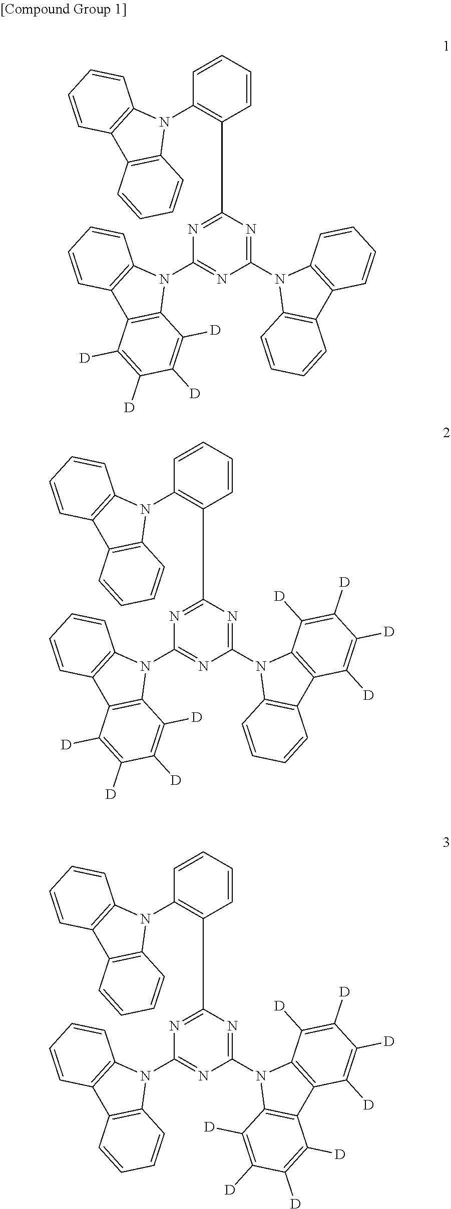 [SMILES: [2H]c1c([2H])c([2H])c2c(c1[2H])c1ccccc1n2-c1nc(-c2ccccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2ccccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1c([2H])c([2H])c([2H])c([2H])c1n2-c1nc(-c2ccccc2-n2c3ccccc3c3ccccc32)nc(-n2c3ccccc3c3ccccc32)n1]

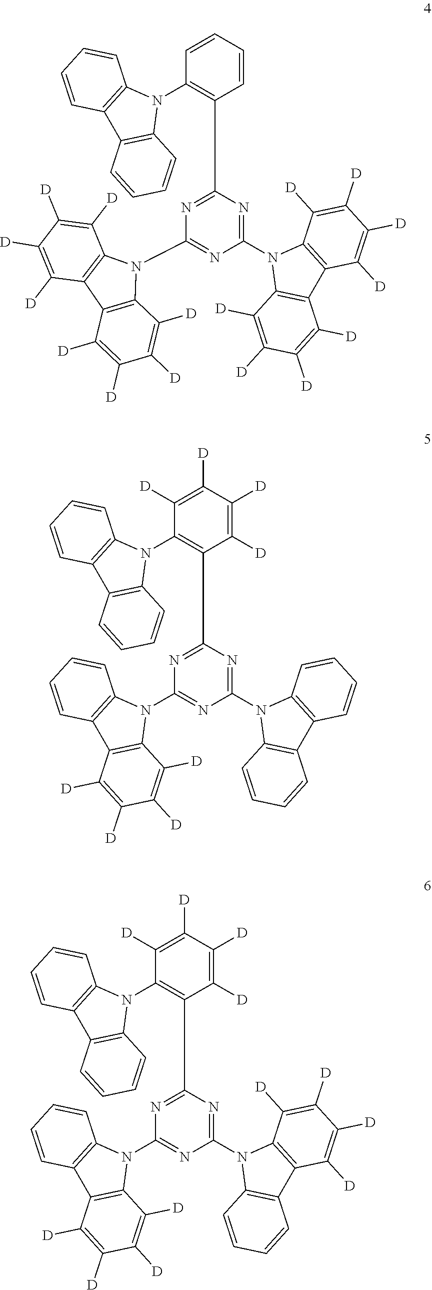 [SMILES: [2H]c1c([2H])c([2H])c2c(c1[2H])c1c([2H])c([2H])c([2H])c([2H])c1n2-c1nc(-c2ccccc2-n2c3ccccc3c3ccccc32)nc(-n2c3c([2H])c([2H])c([2H])c([2H])c3c3c([2H])c([2H])c([2H])c([2H])c32)n1 ; [2H]c1c([2H])c([2H])c(-n2c3ccccc3c3ccccc32)c(-c2nc(-n3c4ccccc4c4ccccc43)nc(-n3c4ccccc4c4c([2H])c([2H])c([2H])c([2H])c43)n2)c1[2H] ; [2H]c1c([2H])c([2H])c(-n2c3ccccc3c3ccccc32)c(-c2nc(-n3c4ccccc4c4c([2H])c([2H])c([2H])c([2H])c43)nc(-n3c4ccccc4c4c([2H])c([2H])c([2H])c([2H])c43)n2)c1[2H]]

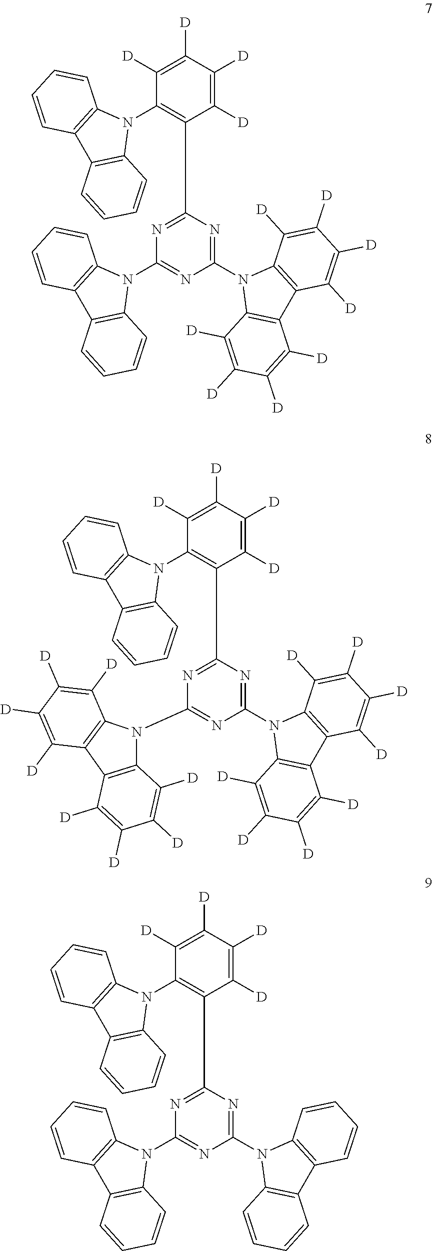 [SMILES: [2H]c1c([2H])c([2H])c(-n2c3ccccc3c3ccccc32)c(-c2nc(-n3c4ccccc4c4ccccc43)nc(-n3c4c([2H])c([2H])c([2H])c([2H])c4c4c([2H])c([2H])c([2H])c([2H])c43)n2)c1[2H] ; [2H]c1c([2H])c([2H])c(-n2c3ccccc3c3ccccc32)c(-c2nc(-n3c4c([2H])c([2H])c([2H])c([2H])c4c4c([2H])c([2H])c([2H])c([2H])c43)nc(-n3c4c([2H])c([2H])c([2H])c([2H])c4c4c([2H])c([2H])c([2H])c([2H])c43)n2)c1[2H] ; [2H]c1c([2H])c([2H])c(-n2c3ccccc3c3ccccc32)c(-c2nc(-n3c4ccccc4c4ccccc43)nc(-n3c4ccccc4c4ccccc43)n2)c1[2H]]

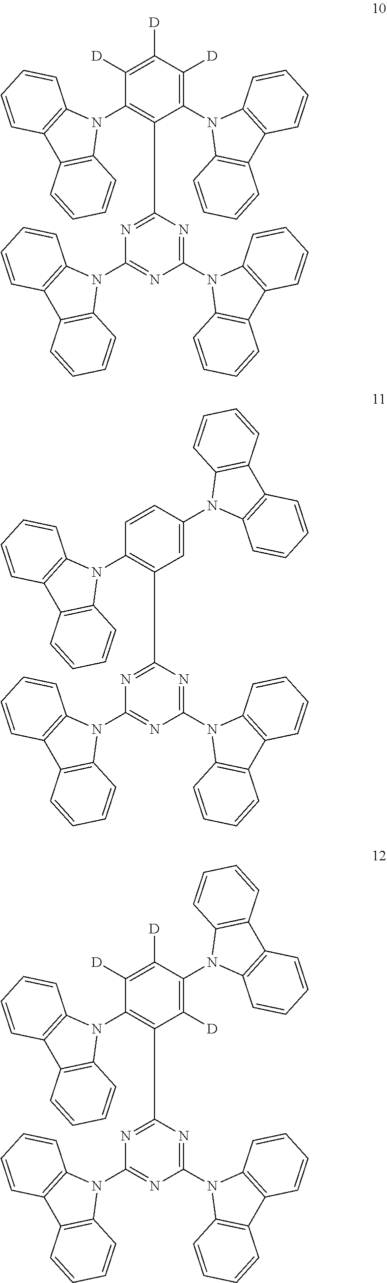

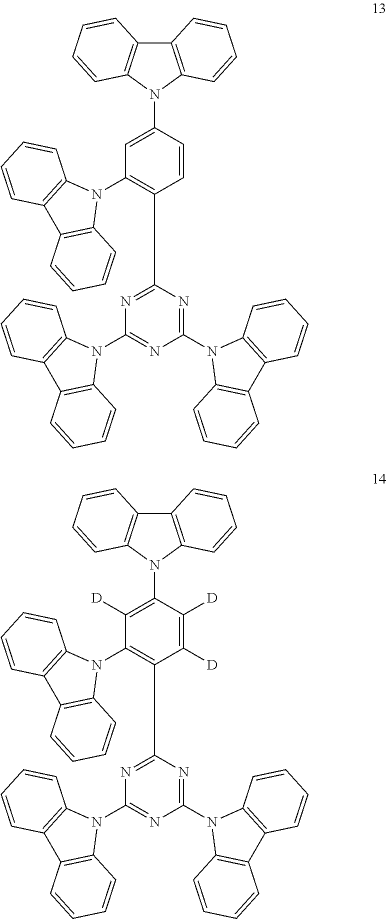 [SMILES: c1ccc2c(c1)c1ccccc1n2-c1ccc(-c2nc(-n3c4ccccc4c4ccccc43)nc(-n3c4ccccc4c4ccccc43)n2)c(-n2c3ccccc3c3ccccc32)c1 ; [2H]c1c([2H])c(-n2c3ccccc3c3ccccc32)c([2H])c(-n2c3ccccc3c3ccccc32)c1-c1nc(-n2c3ccccc3c3ccccc32)nc(-n2c3ccccc3c3ccccc32)n1]

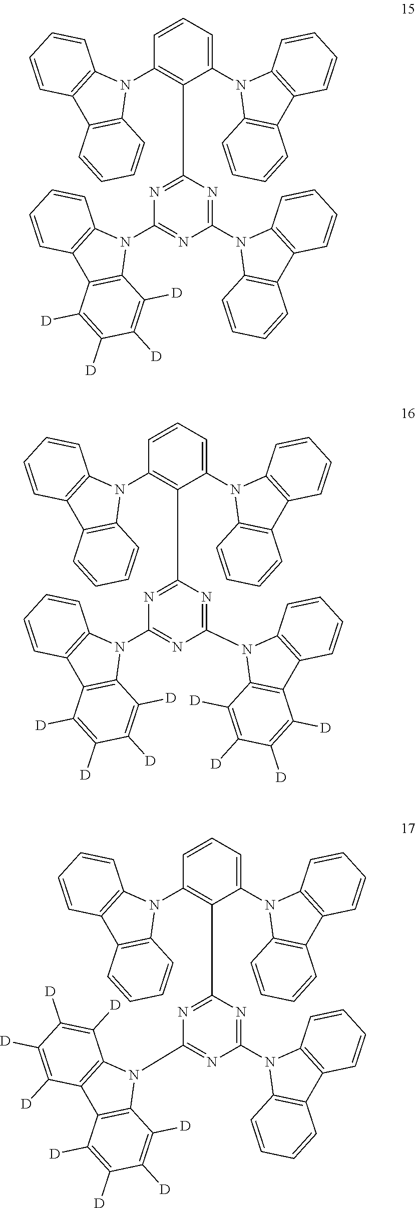 [SMILES: [2H]c1c([2H])c([2H])c2c(c1[2H])c1ccccc1n2-c1nc(-c2c(-n3c4ccccc4c4ccccc43)cccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2c(-n3c4ccccc4c4ccccc43)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1c([2H])c([2H])c([2H])c([2H])c1n2-c1nc(-c2c(-n3c4ccccc4c4ccccc43)cccc2-n2c3ccccc3c3ccccc32)nc(-n2c3ccccc3c3ccccc32)n1]

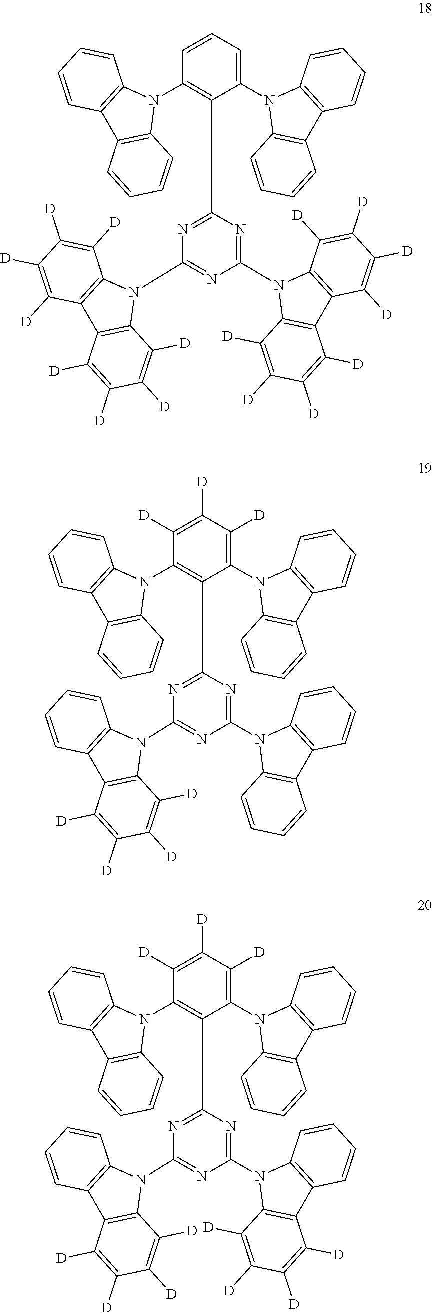

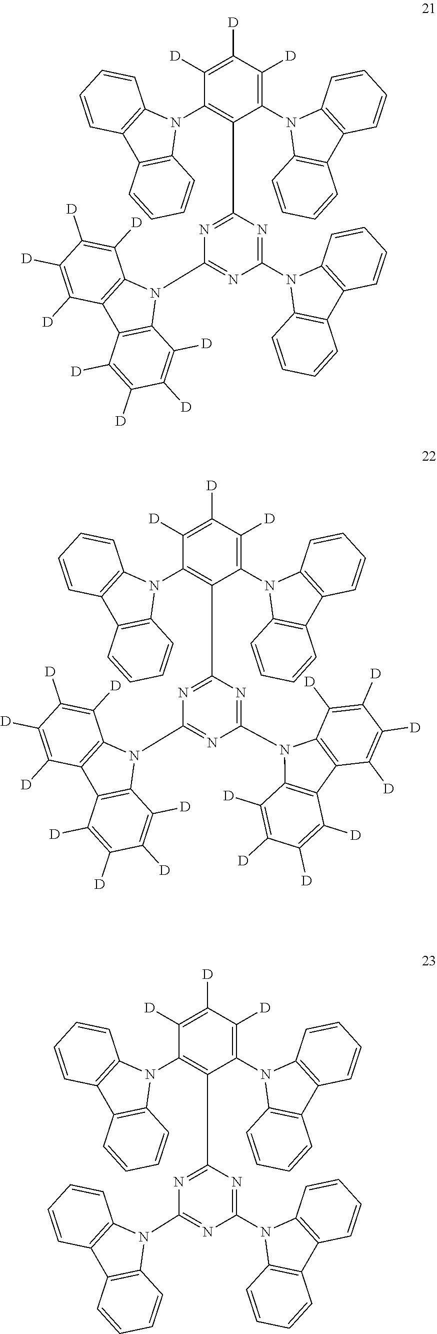

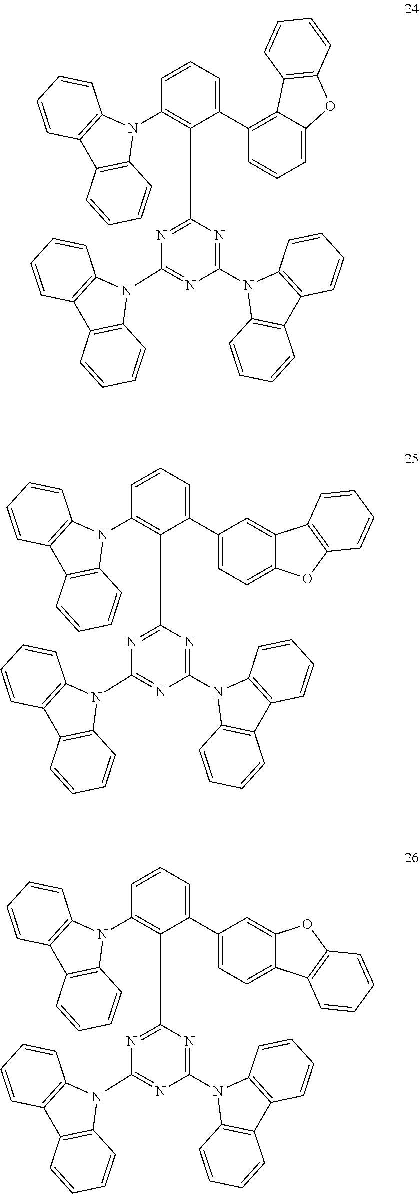 [SMILES: c1cc(-c2cccc3oc4ccccc4c23)c(-c2nc(-n3c4ccccc4c4ccccc43)nc(-n3c4ccccc4c4ccccc43)n2)c(-n2c3ccccc3c3ccccc32)c1 ; c1cc(-c2ccc3oc4ccccc4c3c2)c(-c2nc(-n3c4ccccc4c4ccccc43)nc(-n3c4ccccc4c4ccccc43)n2)c(-n2c3ccccc3c3ccccc32)c1 ; c1cc(-c2ccc3c(c2)oc2ccccc23)c(-c2nc(-n3c4ccccc4c4ccccc43)nc(-n3c4ccccc4c4ccccc43)n2)c(-n2c3ccccc3c3ccccc32)c1]

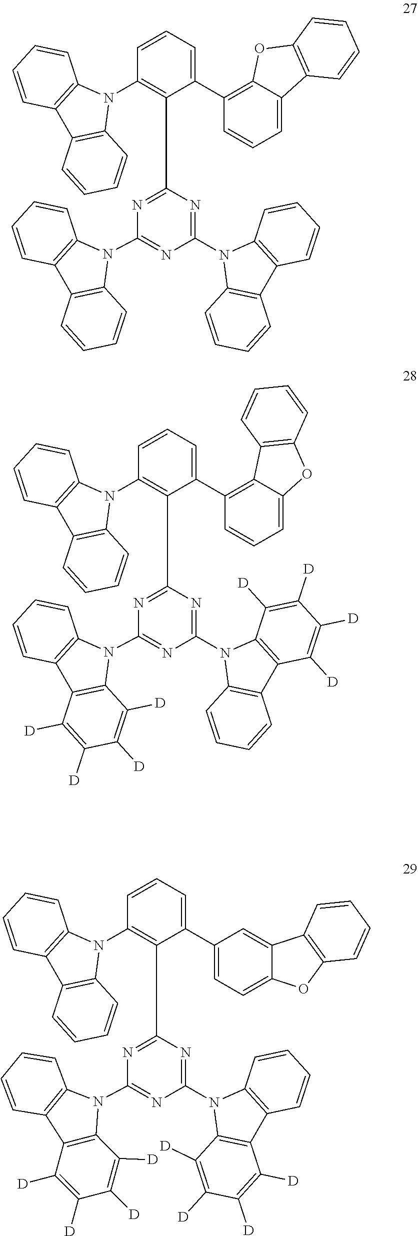

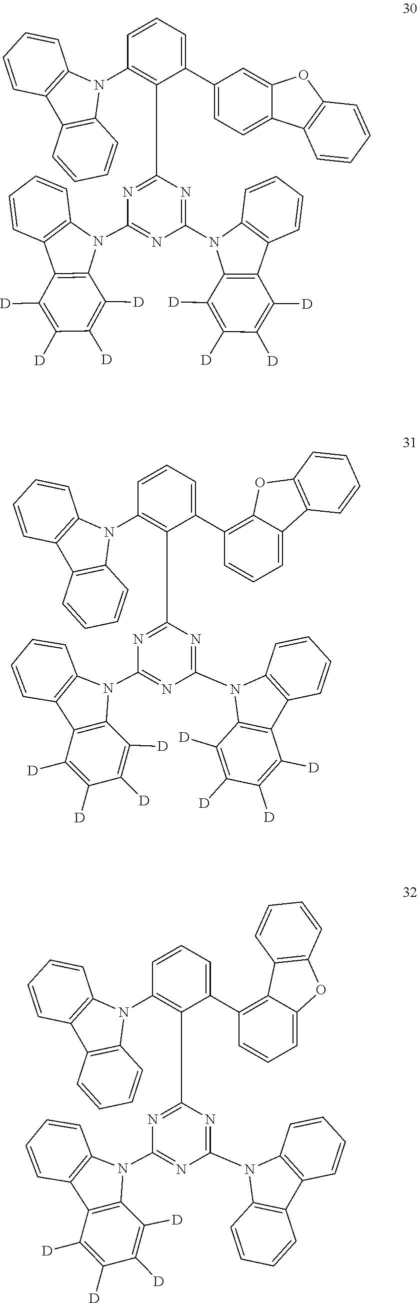 [SMILES: [2H]c1c([2H])c([2H])c2c(c1[2H])c1ccccc1n2-c1nc(-c2c(-c3ccc4c(c3)oc3ccccc34)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2c(-c3cccc4c3oc3ccccc34)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2c(-c3cccc4oc5ccccc5c34)cccc2-n2c3ccccc3c3ccccc32)nc(-n2c3ccccc3c3ccccc32)n1]

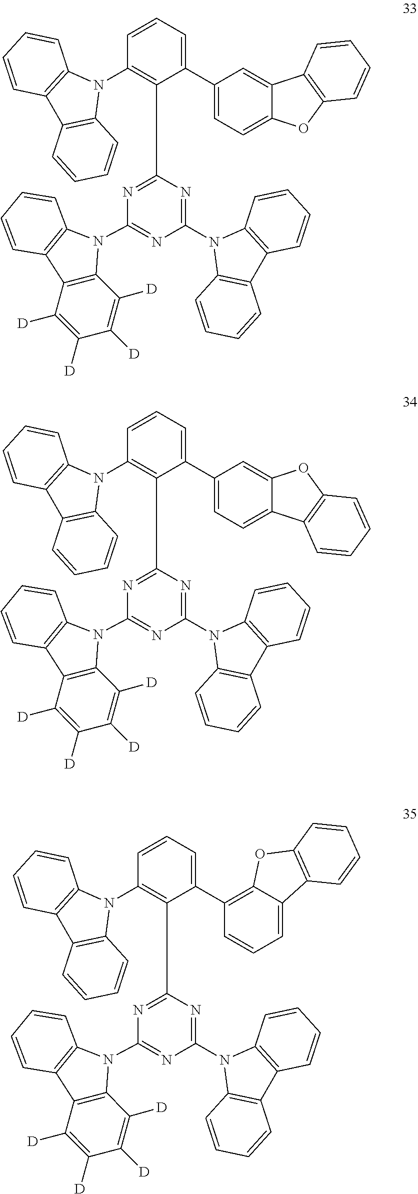 [SMILES: [2H]c1c([2H])c([2H])c2c(c1[2H])c1ccccc1n2-c1nc(-c2c(-c3ccc4oc5ccccc5c4c3)cccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2c(-c3ccc4c(c3)oc3ccccc34)cccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2c(-c3cccc4c3oc3ccccc34)cccc2-n2c3ccccc3c3ccccc32)nc(-n2c3ccccc3c3ccccc32)n1]

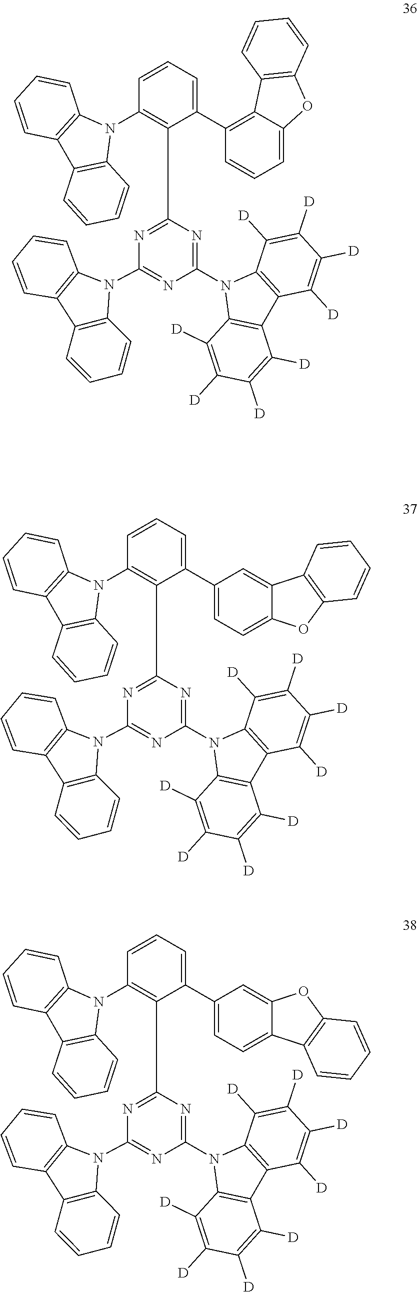 [SMILES: [2H]c1c([2H])c([2H])c2c(c1[2H])c1c([2H])c([2H])c([2H])c([2H])c1n2-c1nc(-c2c(-c3cccc4oc5ccccc5c34)cccc2-n2c3ccccc3c3ccccc32)nc(-n2c3ccccc3c3ccccc32)n1 ; [2H]c1c([2H])c([2H])c2c(c1[2H])c1c([2H])c([2H])c([2H])c([2H])c1n2-c1nc(-c2c(-c3ccc4oc5ccccc5c4c3)cccc2-n2c3ccccc3c3ccccc32)nc(-n2c3ccccc3c3ccccc32)n1 ; [2H]c1c([2H])c([2H])c2c(c1[2H])c1c([2H])c([2H])c([2H])c([2H])c1n2-c1nc(-c2c(-c3ccc4c(c3)oc3ccccc34)cccc2-n2c3ccccc3c3ccccc32)nc(-n2c3ccccc3c3ccccc32)n1]

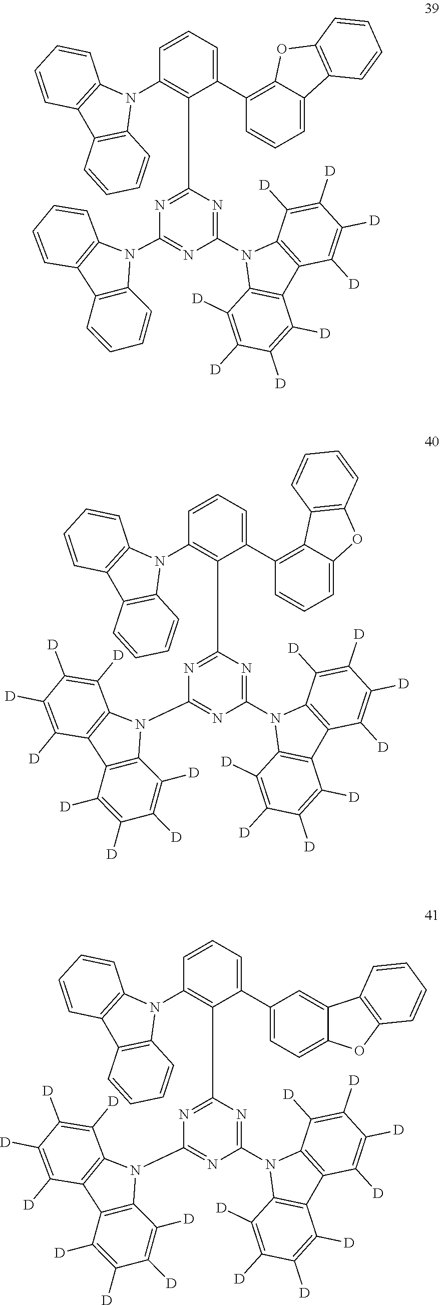

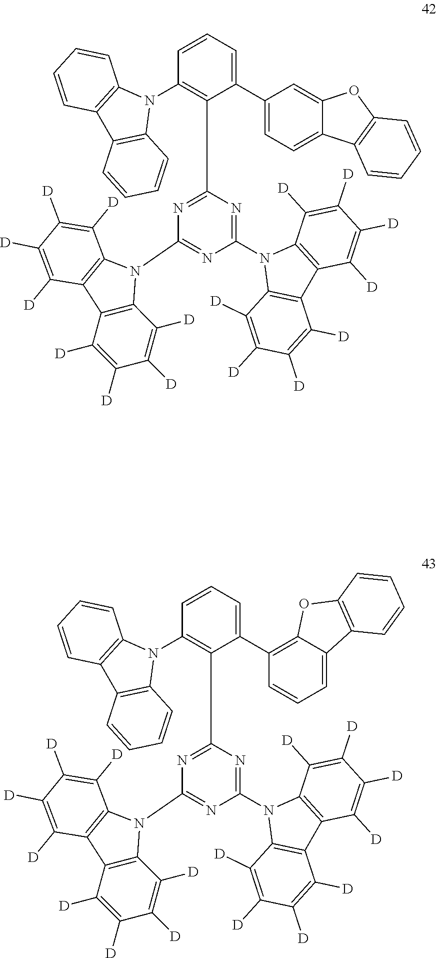 [SMILES: [2H]c1c([2H])c([2H])c2c(c1[2H])c1c([2H])c([2H])c([2H])c([2H])c1n2-c1nc(-c2c(-c3ccc4c(c3)oc3ccccc34)cccc2-n2c3ccccc3c3ccccc32)nc(-n2c3c([2H])c([2H])c([2H])c([2H])c3c3c([2H])c([2H])c([2H])c([2H])c32)n1 ; [2H]c1c([2H])c([2H])c2c(c1[2H])c1c([2H])c([2H])c([2H])c([2H])c1n2-c1nc(-c2c(-c3cccc4c3oc3ccccc34)cccc2-n2c3ccccc3c3ccccc32)nc(-n2c3c([2H])c([2H])c([2H])c([2H])c3c3c([2H])c([2H])c([2H])c([2H])c32)n1]

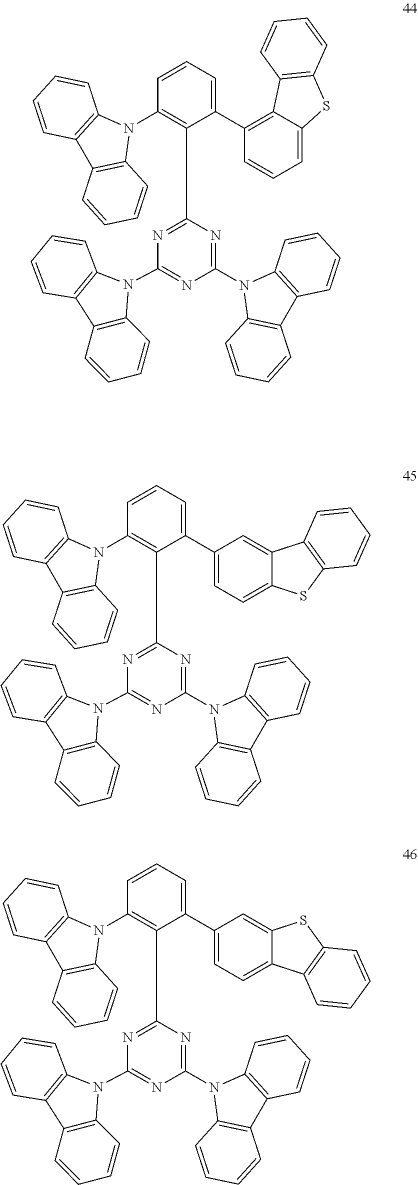 [SMILES: c1cc(-c2cccc3sc4ccccc4c23)c(-c2nc(-n3c4ccccc4c4ccccc43)nc(-n3c4ccccc4c4ccccc43)n2)c(-n2c3ccccc3c3ccccc32)c1 ; c1cc(-c2ccc3sc4ccccc4c3c2)c(-c2nc(-n3c4ccccc4c4ccccc43)nc(-n3c4ccccc4c4ccccc43)n2)c(-n2c3ccccc3c3ccccc32)c1 ; c1cc(-c2ccc3c(c2)sc2ccccc23)c(-c2nc(-n3c4ccccc4c4ccccc43)nc(-n3c4ccccc4c4ccccc43)n2)c(-n2c3ccccc3c3ccccc32)c1]

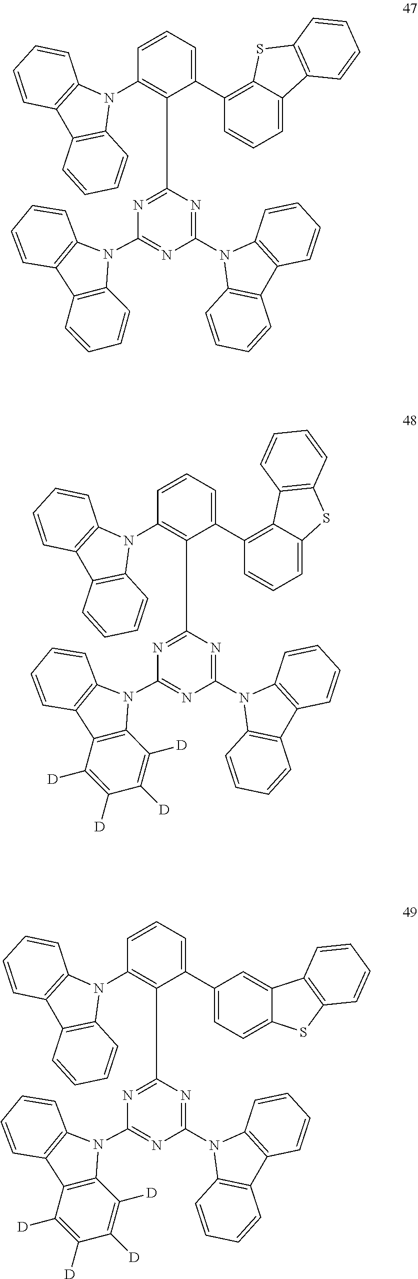 [SMILES: c1cc(-c2cccc3c2sc2ccccc23)c(-c2nc(-n3c4ccccc4c4ccccc43)nc(-n3c4ccccc4c4ccccc43)n2)c(-n2c3ccccc3c3ccccc32)c1 ; [2H]c1c([2H])c([2H])c2c(c1[2H])c1ccccc1n2-c1nc(-c2c(-c3cccc4sc5ccccc5c34)cccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2c(-c3ccc4sc5ccccc5c4c3)cccc2-n2c3ccccc3c3ccccc32)nc(-n2c3ccccc3c3ccccc32)n1]

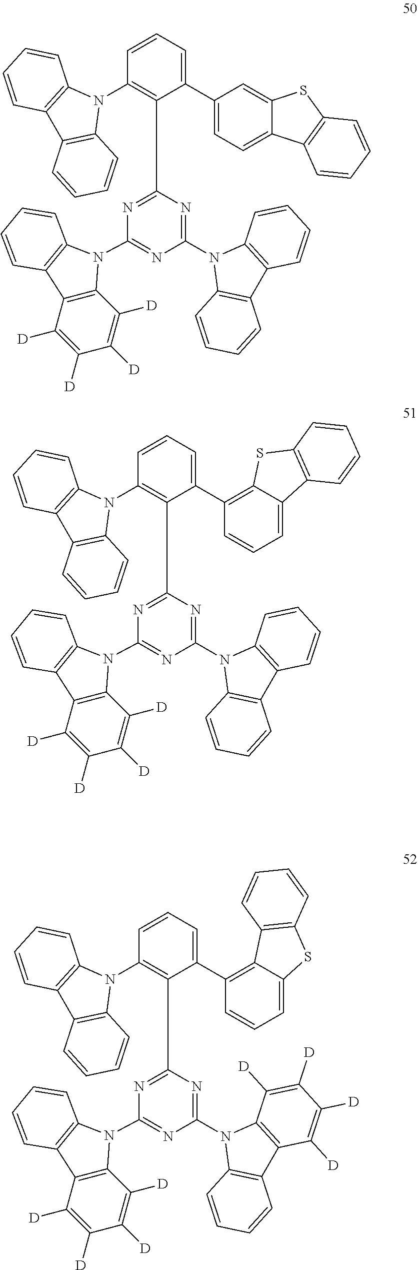 [SMILES: [2H]c1c([2H])c([2H])c2c(c1[2H])c1ccccc1n2-c1nc(-c2c(-c3ccc4c(c3)sc3ccccc34)cccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2c(-c3cccc4c3sc3ccccc34)cccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2c(-c3cccc4sc5ccccc5c34)cccc2-n2c3ccccc3c3ccccc32)nc(-n2c3ccccc3c3c([2H])c([2H])c([2H])c([2H])c32)n1]

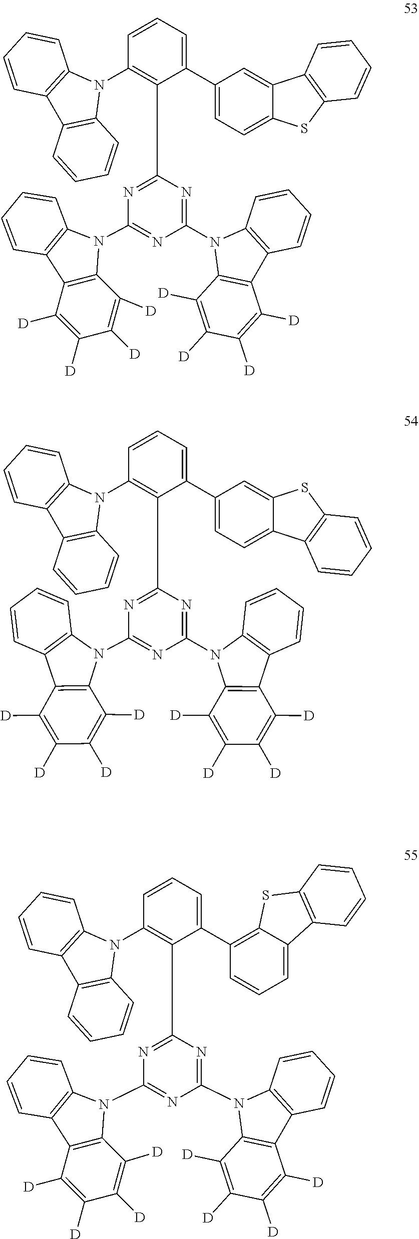 [SMILES: [2H]c1c([2H])c([2H])c2c(c1[2H])c1ccccc1n2-c1nc(-c2c(-c3ccc4sc5ccccc5c4c3)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2c(-c3ccc4c(c3)sc3ccccc34)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2c(-c3cccc4c3sc3ccccc34)cccc2-n2c3ccccc3c3ccccc32)nc(-n2c3ccccc3c3c([2H])c([2H])c([2H])c([2H])c32)n1]

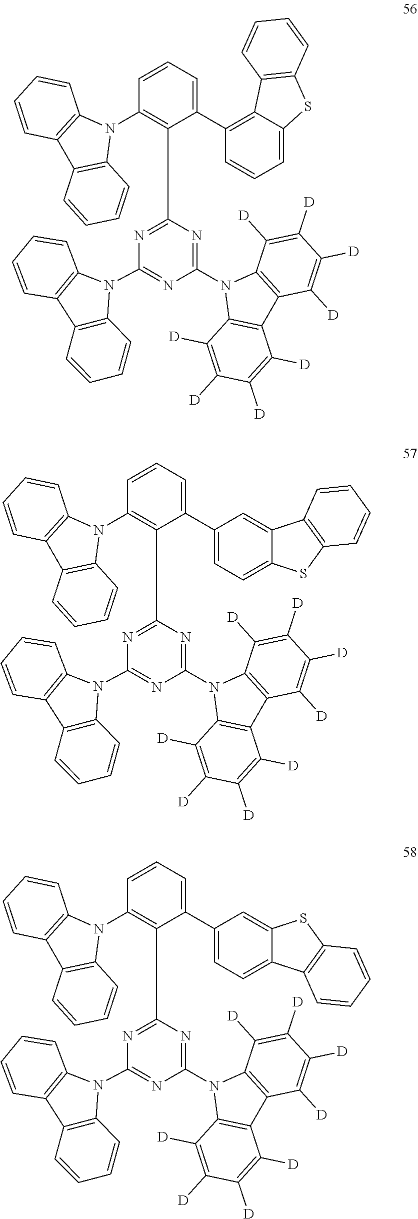 [SMILES: [2H]c1c([2H])c([2H])c2c(c1[2H])c1c([2H])c([2H])c([2H])c([2H])c1n2-c1nc(-c2c(-c3cccc4sc5ccccc5c34)cccc2-n2c3ccccc3c3ccccc32)nc(-n2c3ccccc3c3ccccc32)n1 ; [2H]c1c([2H])c([2H])c2c(c1[2H])c1c([2H])c([2H])c([2H])c([2H])c1n2-c1nc(-c2c(-c3ccc4sc5ccccc5c4c3)cccc2-n2c3ccccc3c3ccccc32)nc(-n2c3ccccc3c3ccccc32)n1 ; [2H]c1c([2H])c([2H])c2c(c1[2H])c1c([2H])c([2H])c([2H])c([2H])c1n2-c1nc(-c2c(-c3ccc4c(c3)sc3ccccc34)cccc2-n2c3ccccc3c3ccccc32)nc(-n2c3ccccc3c3ccccc32)n1]

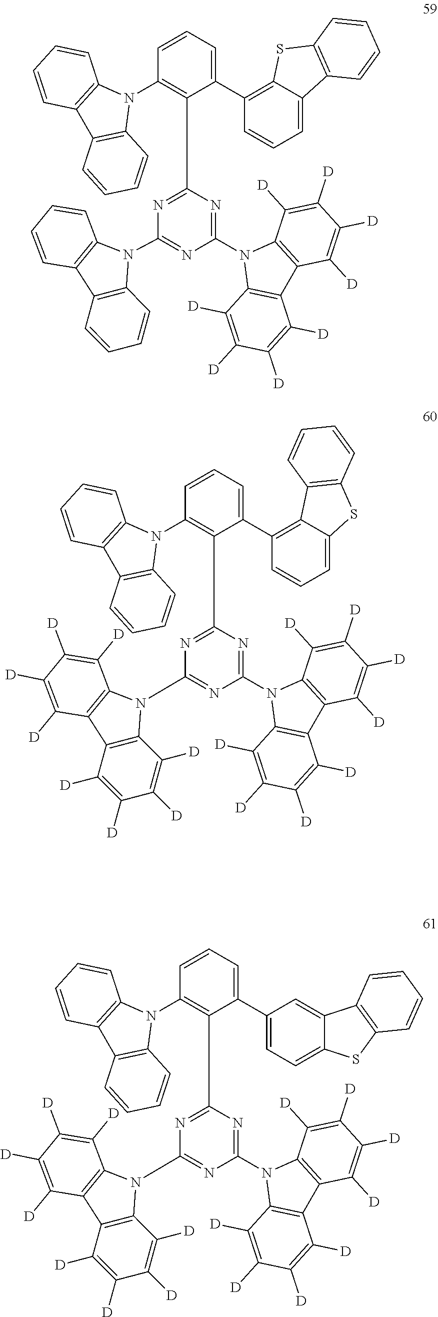

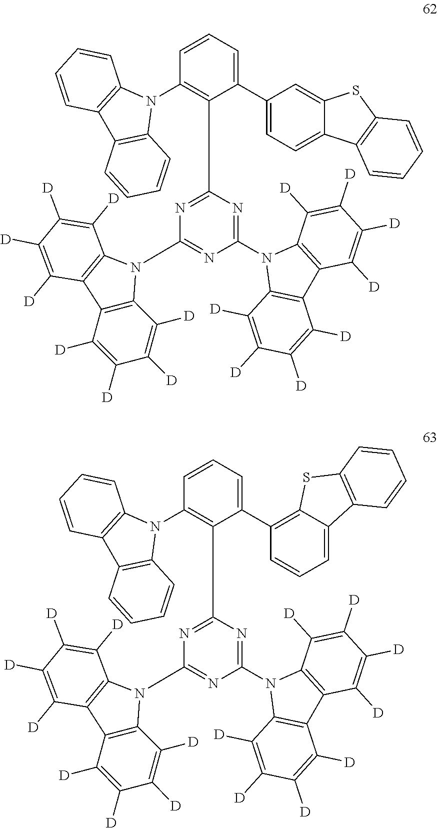 [SMILES: [2H]c1c([2H])c([2H])c2c(c1[2H])c1c([2H])c([2H])c([2H])c([2H])c1n2-c1nc(-c2c(-c3ccc4c(c3)sc3ccccc34)cccc2-n2c3ccccc3c3ccccc32)nc(-n2c3c([2H])c([2H])c([2H])c([2H])c3c3c([2H])c([2H])c([2H])c([2H])c32)n1 ; [2H]c1c([2H])c([2H])c2c(c1[2H])c1c([2H])c([2H])c([2H])c([2H])c1n2-c1nc(-c2c(-c3cccc4c3sc3ccccc34)cccc2-n2c3ccccc3c3ccccc32)nc(-n2c3c([2H])c([2H])c([2H])c([2H])c3c3c([2H])c([2H])c([2H])c([2H])c32)n1]

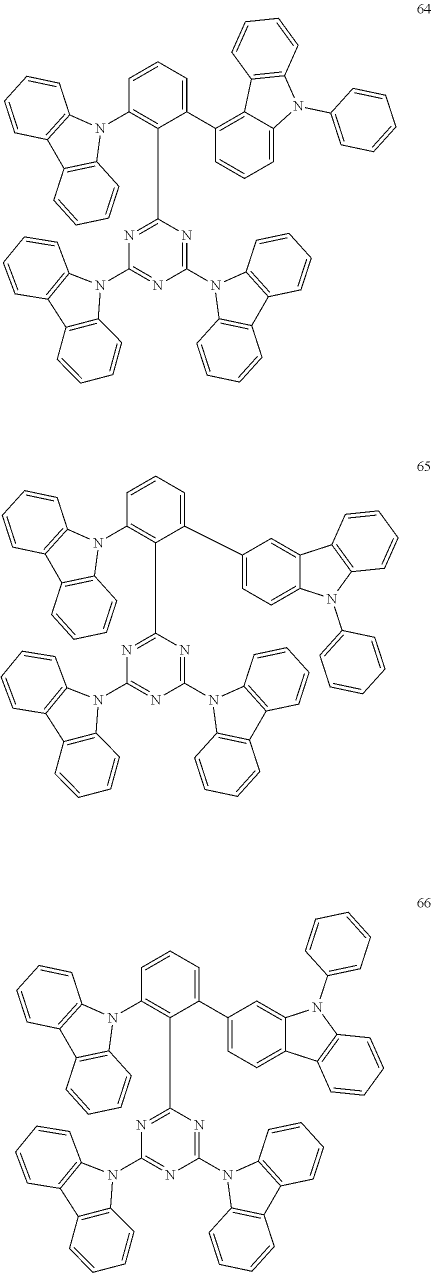 [SMILES: c1ccc(-n2c3ccccc3c3c(-c4cccc(-n5c6ccccc6c6ccccc65)c4-c4nc(-n5c6ccccc6c6ccccc65)nc(-n5c6ccccc6c6ccccc65)n4)cccc32)cc1 ; c1ccc(-n2c3ccccc3c3cc(-c4cccc(-n5c6ccccc6c6ccccc65)c4-c4nc(-n5c6ccccc6c6ccccc65)nc(-n5c6ccccc6c6ccccc65)n4)ccc32)cc1 ; c1ccc(-n2c3ccccc3c3ccc(-c4cccc(-n5c6ccccc6c6ccccc65)c4-c4nc(-n5c6ccccc6c6ccccc65)nc(-n5c6ccccc6c6ccccc65)n4)cc32)cc1]

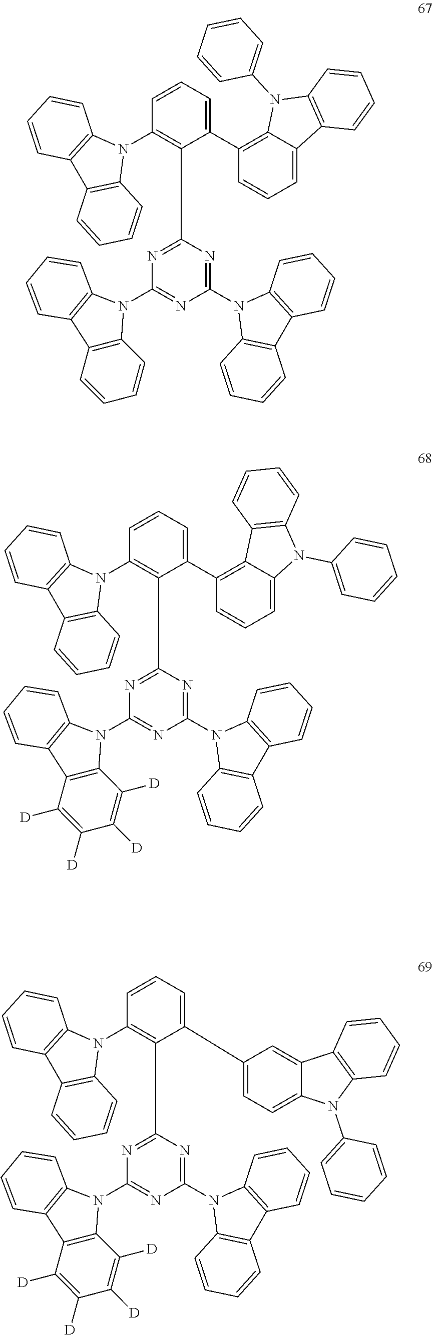 [SMILES: c1ccc(-n2c3ccccc3c3cccc(-c4cccc(-n5c6ccccc6c6ccccc65)c4-c4nc(-n5c6ccccc6c6ccccc65)nc(-n5c6ccccc6c6ccccc65)n4)c32)cc1 ; [2H]c1c([2H])c([2H])c2c(c1[2H])c1ccccc1n2-c1nc(-c2c(-c3cccc4c3c3ccccc3n4-c3ccccc3)cccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2c(-c3ccc4c(c3)c3ccccc3n4-c3ccccc3)cccc2-n2c3ccccc3c3ccccc32)nc(-n2c3ccccc3c3ccccc32)n1]

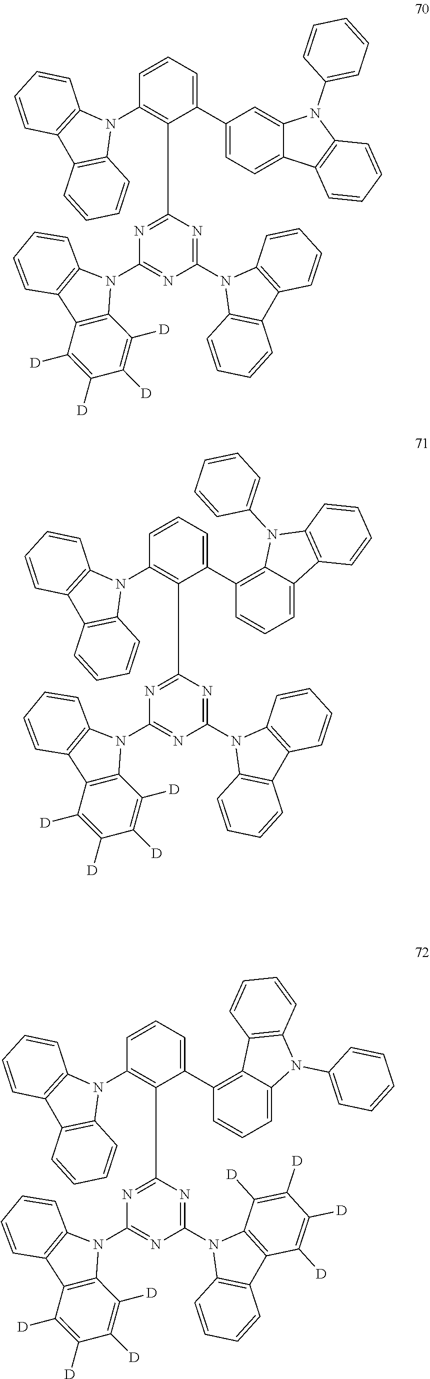 [SMILES: [2H]c1c([2H])c([2H])c2c(c1[2H])c1ccccc1n2-c1nc(-c2c(-c3ccc4c5ccccc5n(-c5ccccc5)c4c3)cccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2c(-c3cccc4c5ccccc5n(-c5ccccc5)c34)cccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2c(-c3cccc4c3c3ccccc3n4-c3ccccc3)cccc2-n2c3ccccc3c3ccccc32)nc(-n2c3ccccc3c3c([2H])c([2H])c([2H])c([2H])c32)n1]

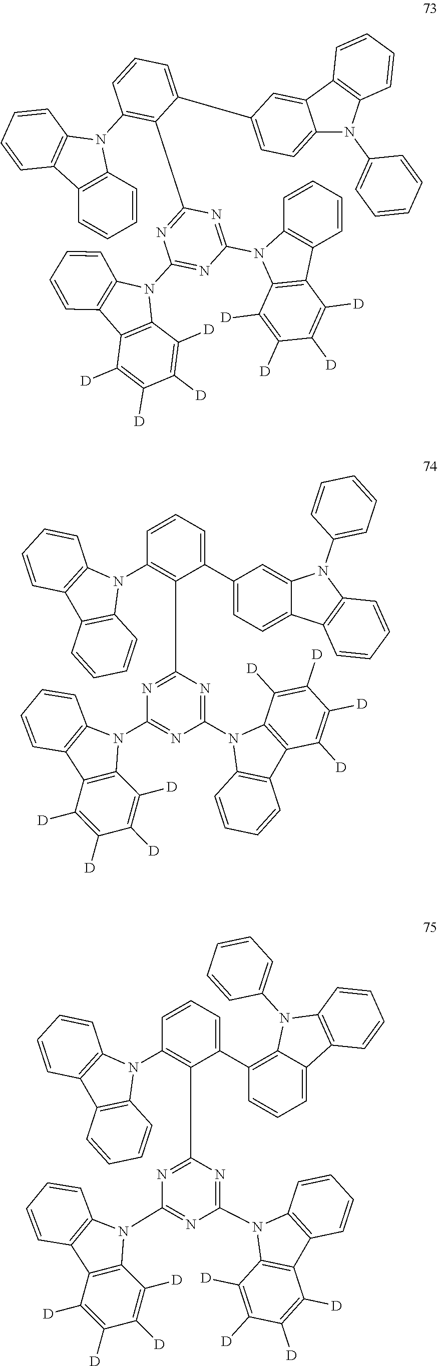 [SMILES: [2H]c1c([2H])c([2H])c2c(c1[2H])c1ccccc1n2-c1nc(-c2c(-c3ccc4c(c3)c3ccccc3n4-c3ccccc3)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2c(-c3ccc4c5ccccc5n(-c5ccccc5)c4c3)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2c(-c3cccc4c5ccccc5n(-c5ccccc5)c34)cccc2-n2c3ccccc3c3ccccc32)nc(-n2c3ccccc3c3c([2H])c([2H])c([2H])c([2H])c32)n1]

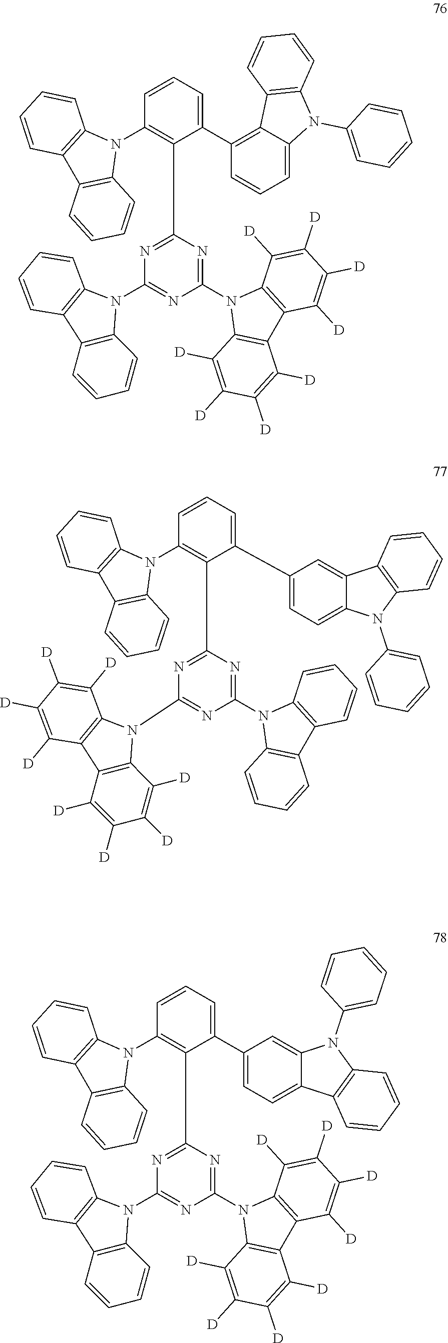 [SMILES: [2H]c1c([2H])c([2H])c2c(c1[2H])c1c([2H])c([2H])c([2H])c([2H])c1n2-c1nc(-c2c(-c3cccc4c3c3ccccc3n4-c3ccccc3)cccc2-n2c3ccccc3c3ccccc32)nc(-n2c3ccccc3c3ccccc32)n1 ; [2H]c1c([2H])c([2H])c2c(c1[2H])c1c([2H])c([2H])c([2H])c([2H])c1n2-c1nc(-c2c(-c3ccc4c(c3)c3ccccc3n4-c3ccccc3)cccc2-n2c3ccccc3c3ccccc32)nc(-n2c3ccccc3c3ccccc32)n1 ; [2H]c1c([2H])c([2H])c2c(c1[2H])c1c([2H])c([2H])c([2H])c([2H])c1n2-c1nc(-c2c(-c3ccc4c5ccccc5n(-c5ccccc5)c4c3)cccc2-n2c3ccccc3c3ccccc32)nc(-n2c3ccccc3c3ccccc32)n1]

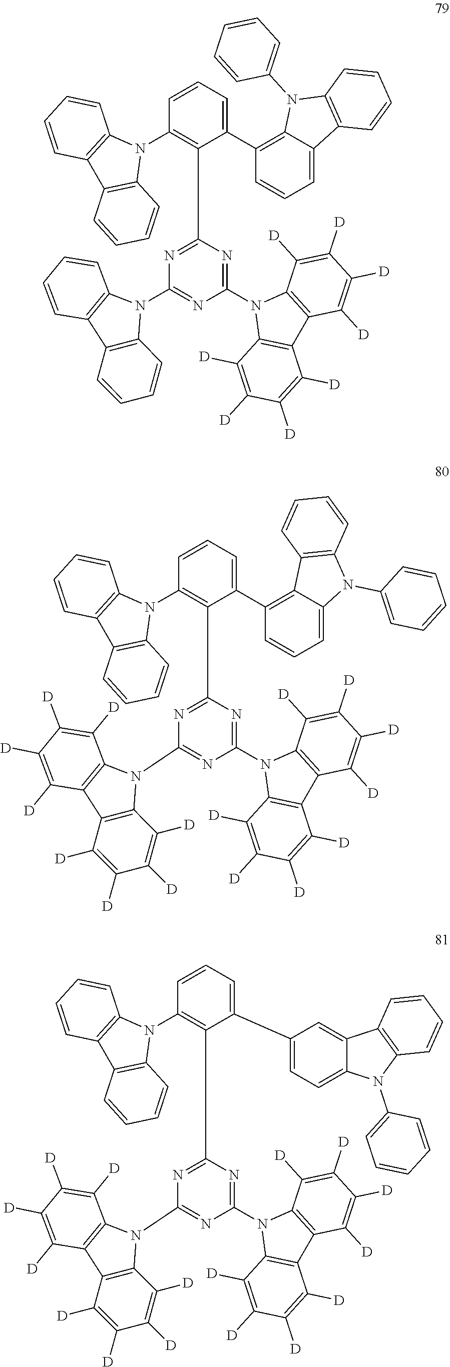

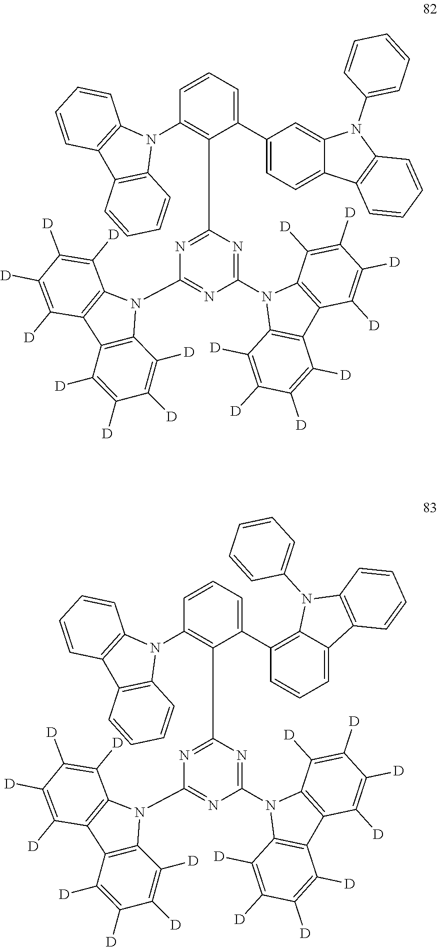 [SMILES: [2H]c1c([2H])c([2H])c2c(c1[2H])c1c([2H])c([2H])c([2H])c([2H])c1n2-c1nc(-c2c(-c3ccc4c5ccccc5n(-c5ccccc5)c4c3)cccc2-n2c3ccccc3c3ccccc32)nc(-n2c3c([2H])c([2H])c([2H])c([2H])c3c3c([2H])c([2H])c([2H])c([2H])c32)n1 ; [2H]c1c([2H])c([2H])c2c(c1[2H])c1c([2H])c([2H])c([2H])c([2H])c1n2-c1nc(-c2c(-c3cccc4c5ccccc5n(-c5ccccc5)c34)cccc2-n2c3ccccc3c3ccccc32)nc(-n2c3c([2H])c([2H])c([2H])c([2H])c3c3c([2H])c([2H])c([2H])c([2H])c32)n1]

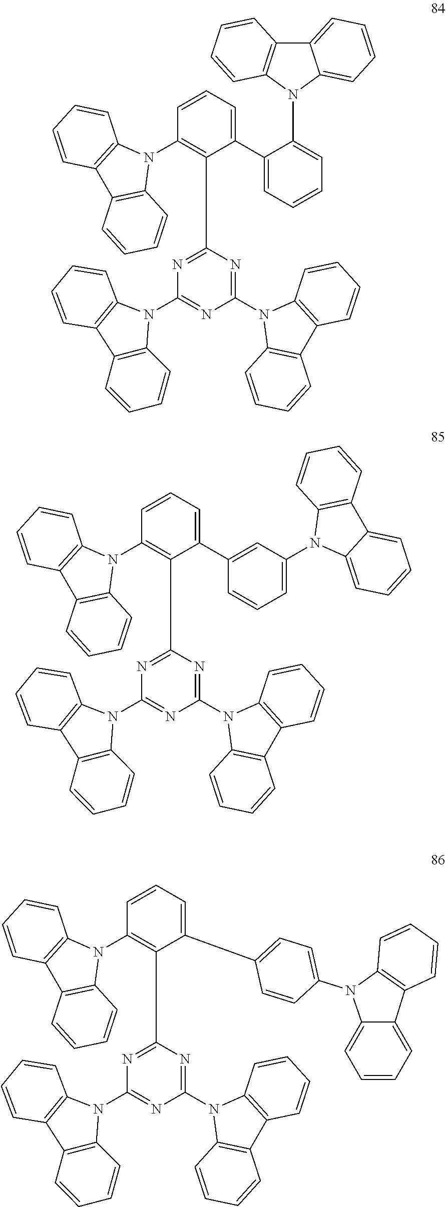

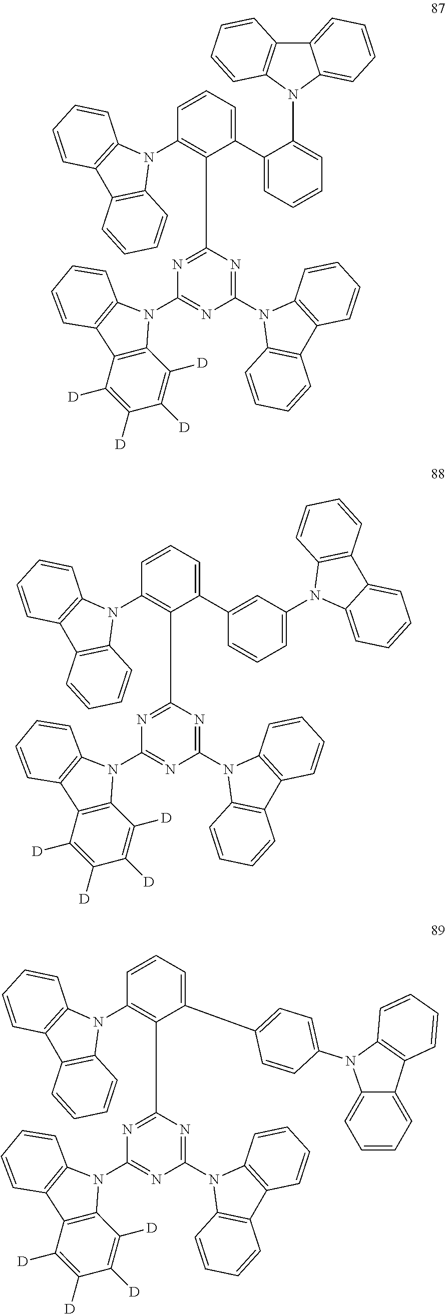 [SMILES: [2H]c1c([2H])c([2H])c2c(c1[2H])c1ccccc1n2-c1nc(-c2c(-c3ccccc3-n3c4ccccc4c4ccccc43)cccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2c(-c3cccc(-n4c5ccccc5c5ccccc54)c3)cccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2c(-c3ccc(-n4c5ccccc5c5ccccc54)cc3)cccc2-n2c3ccccc3c3ccccc32)nc(-n2c3ccccc3c3ccccc32)n1]

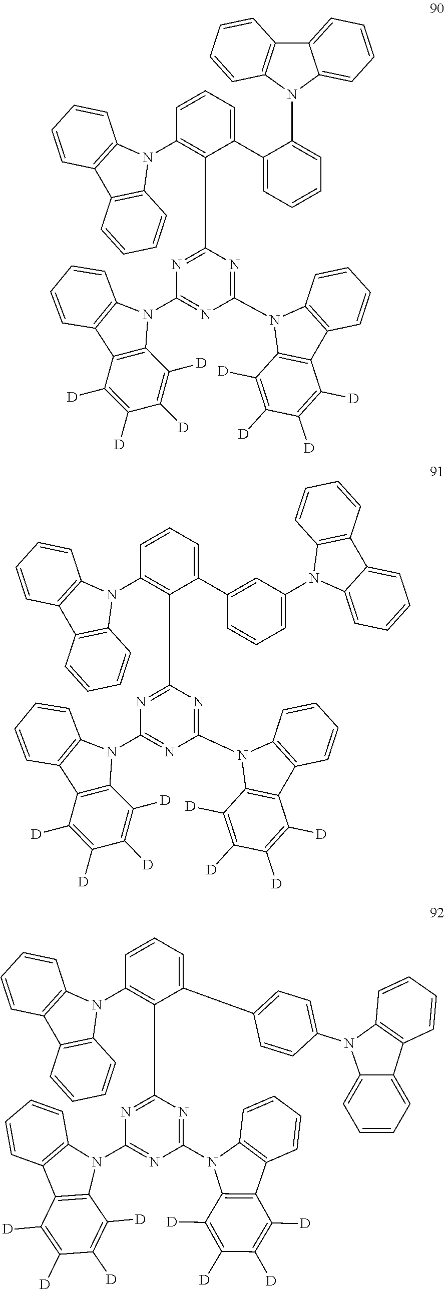

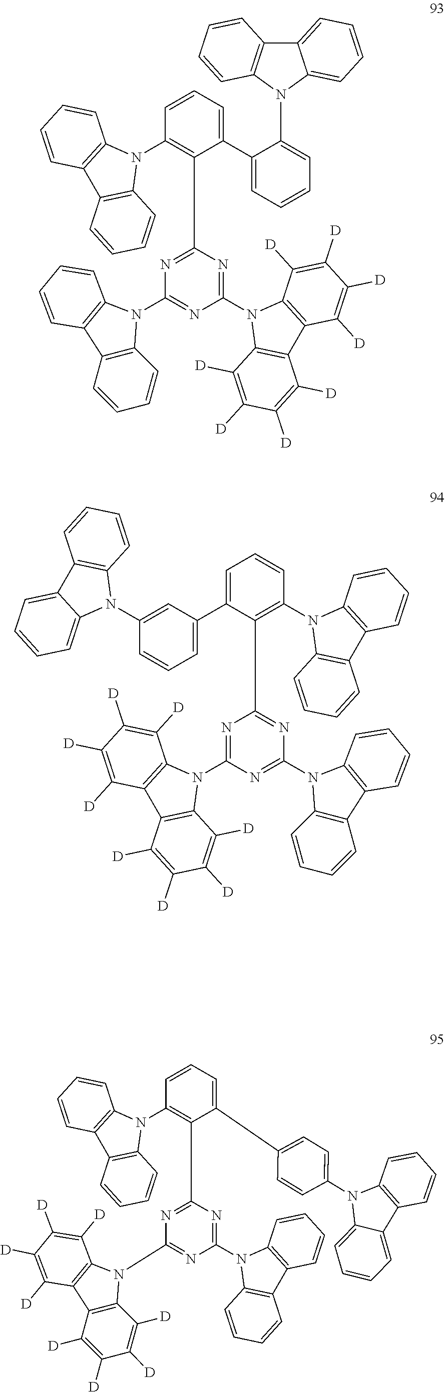 [SMILES: [2H]c1c([2H])c([2H])c2c(c1[2H])c1c([2H])c([2H])c([2H])c([2H])c1n2-c1nc(-c2c(-c3ccccc3-n3c4ccccc4c4ccccc43)cccc2-n2c3ccccc3c3ccccc32)nc(-n2c3ccccc3c3ccccc32)n1 ; [2H]c1c([2H])c([2H])c2c(c1[2H])c1c([2H])c([2H])c([2H])c([2H])c1n2-c1nc(-c2c(-c3cccc(-n4c5ccccc5c5ccccc54)c3)cccc2-n2c3ccccc3c3ccccc32)nc(-n2c3ccccc3c3ccccc32)n1 ; [2H]c1c([2H])c([2H])c2c(c1[2H])c1c([2H])c([2H])c([2H])c([2H])c1n2-c1nc(-c2c(-c3ccc(-n4c5ccccc5c5ccccc54)cc3)cccc2-n2c3ccccc3c3ccccc32)nc(-n2c3ccccc3c3ccccc32)n1]

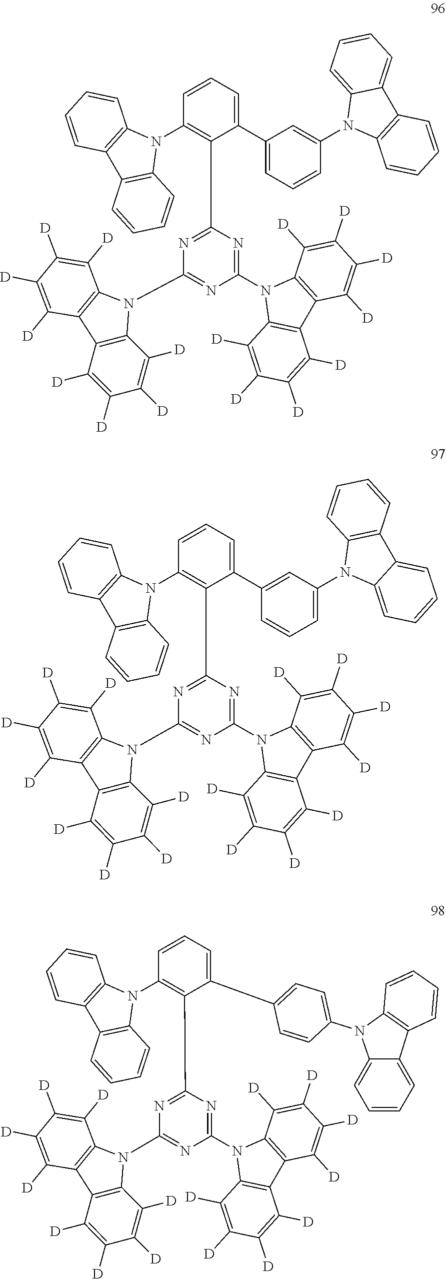

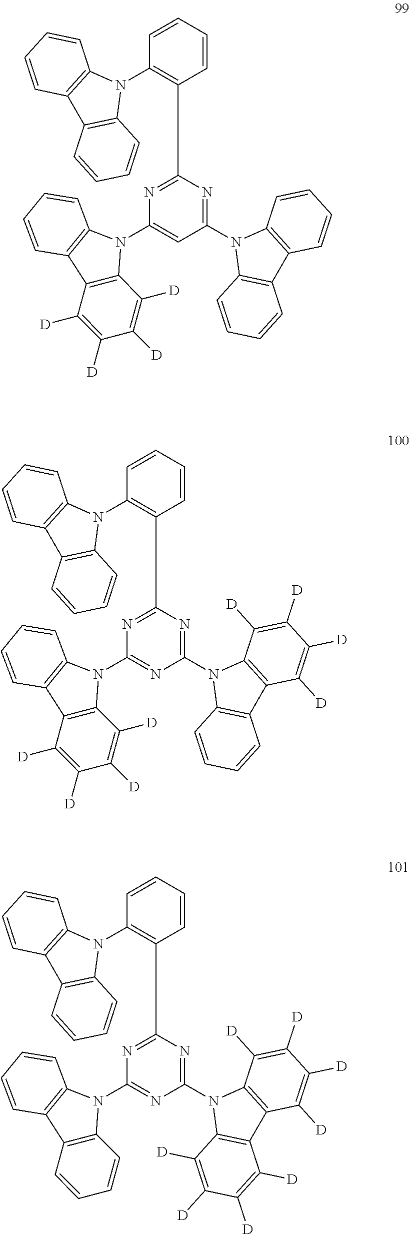 [SMILES: [2H]c1c([2H])c([2H])c2c(c1[2H])c1ccccc1n2-c1cc(-n2c3ccccc3c3ccccc32)nc(-c2ccccc2-n2c3ccccc3c3ccccc32)n1 ; [2H]c1c([2H])c([2H])c2c(c1[2H])c1ccccc1n2-c1nc(-c2ccccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1c([2H])c([2H])c([2H])c([2H])c1n2-c1nc(-c2ccccc2-n2c3ccccc3c3ccccc32)nc(-n2c3ccccc3c3ccccc32)n1]

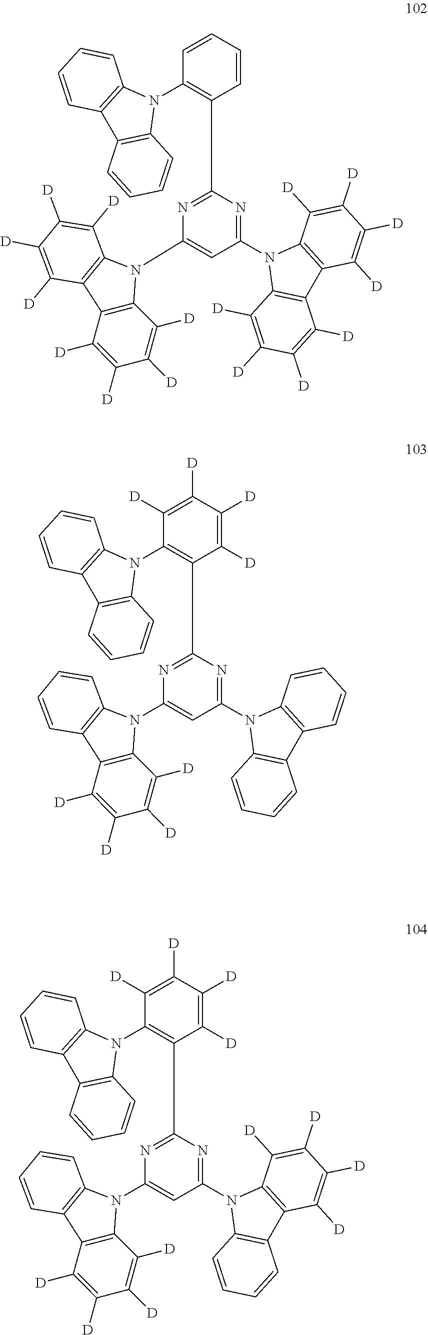 [SMILES: [2H]c1c([2H])c([2H])c2c(c1[2H])c1c([2H])c([2H])c([2H])c([2H])c1n2-c1cc(-n2c3c([2H])c([2H])c([2H])c([2H])c3c3c([2H])c([2H])c([2H])c([2H])c32)nc(-c2ccccc2-n2c3ccccc3c3ccccc32)n1 ; [2H]c1c([2H])c([2H])c(-n2c3ccccc3c3ccccc32)c(-c2nc(-n3c4ccccc4c4ccccc43)cc(-n3c4ccccc4c4c([2H])c([2H])c([2H])c([2H])c43)n2)c1[2H] ; [2H]c1c([2H])c([2H])c(-n2c3ccccc3c3ccccc32)c(-c2nc(-n3c4ccccc4c4c([2H])c([2H])c([2H])c([2H])c43)cc(-n3c4ccccc4c4c([2H])c([2H])c([2H])c([2H])c43)n2)c1[2H]]

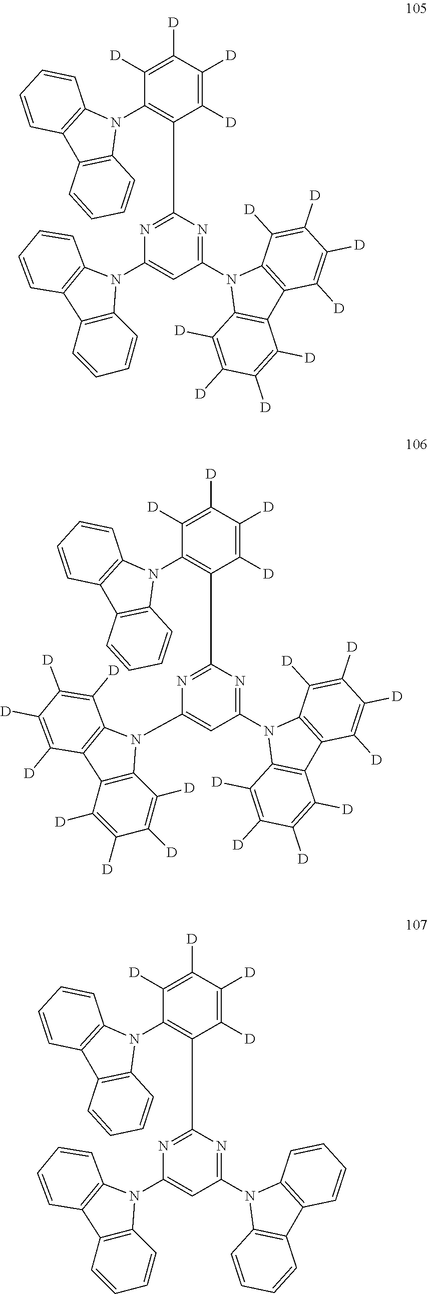 [SMILES: [2H]c1c([2H])c([2H])c(-n2c3ccccc3c3ccccc32)c(-c2nc(-n3c4ccccc4c4ccccc43)cc(-n3c4c([2H])c([2H])c([2H])c([2H])c4c4c([2H])c([2H])c([2H])c([2H])c43)n2)c1[2H] ; [2H]c1c([2H])c([2H])c(-n2c3ccccc3c3ccccc32)c(-c2nc(-n3c4c([2H])c([2H])c([2H])c([2H])c4c4c([2H])c([2H])c([2H])c([2H])c43)cc(-n3c4c([2H])c([2H])c([2H])c([2H])c4c4c([2H])c([2H])c([2H])c([2H])c43)n2)c1[2H] ; [2H]c1c([2H])c([2H])c(-n2c3ccccc3c3ccccc32)c(-c2nc(-n3c4ccccc4c4ccccc43)cc(-n3c4ccccc4c4ccccc43)n2)c1[2H]]

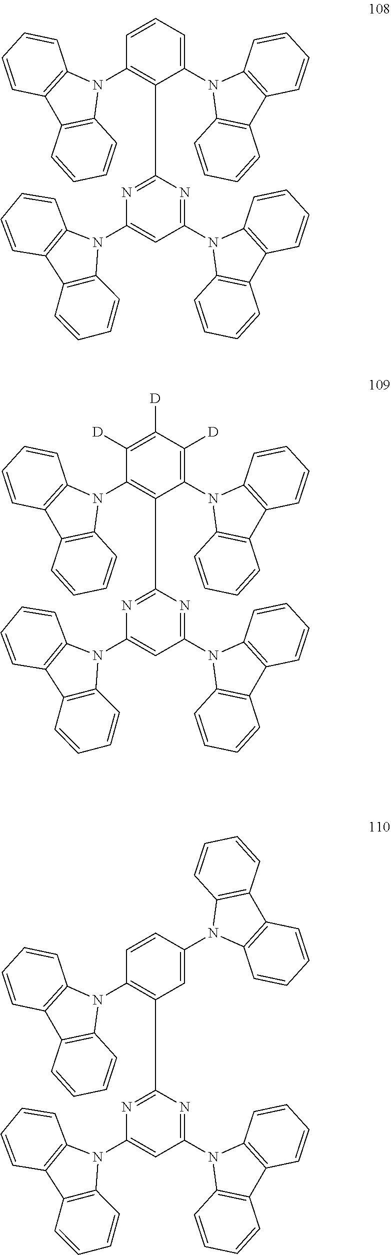 [SMILES: c1cc(-n2c3ccccc3c3ccccc32)c(-c2nc(-n3c4ccccc4c4ccccc43)cc(-n3c4ccccc4c4ccccc43)n2)c(-n2c3ccccc3c3ccccc32)c1 ; [2H]c1c([2H])c(-n2c3ccccc3c3ccccc32)c(-c2nc(-n3c4ccccc4c4ccccc43)cc(-n3c4ccccc4c4ccccc43)n2)c(-n2c3ccccc3c3ccccc32)c1[2H] ; c1ccc2c(c1)c1ccccc1n2-c1ccc(-n2c3ccccc3c3ccccc32)c(-c2nc(-n3c4ccccc4c4ccccc43)cc(-n3c4ccccc4c4ccccc43)n2)c1]

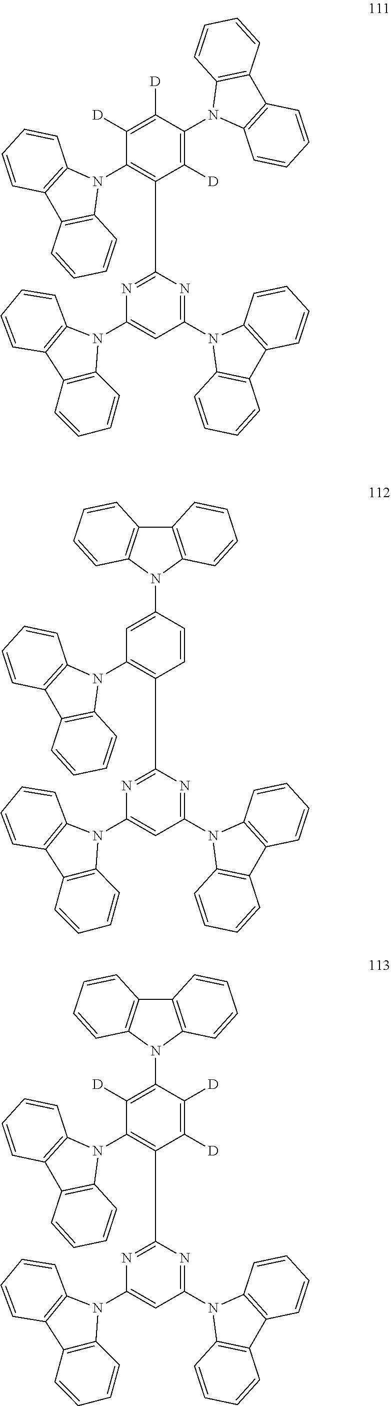 [SMILES: [2H]c1c([2H])c(-n2c3ccccc3c3ccccc32)c(-c2nc(-n3c4ccccc4c4ccccc43)cc(-n3c4ccccc4c4ccccc43)n2)c([2H])c1-n1c2ccccc2c2ccccc21 ; c1ccc2c(c1)c1ccccc1n2-c1ccc(-c2nc(-n3c4ccccc4c4ccccc43)cc(-n3c4ccccc4c4ccccc43)n2)c(-n2c3ccccc3c3ccccc32)c1 ; [2H]c1c([2H])c(-n2c3ccccc3c3ccccc32)c([2H])c(-n2c3ccccc3c3ccccc32)c1-c1nc(-n2c3ccccc3c3ccccc32)cc(-n2c3ccccc3c3ccccc32)n1]

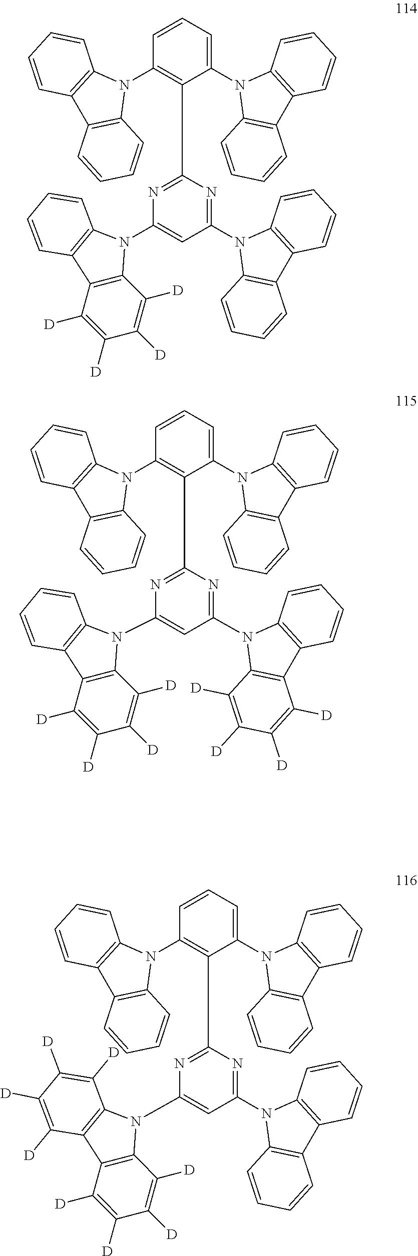 [SMILES: [2H]c1c([2H])c([2H])c2c(c1[2H])c1ccccc1n2-c1cc(-n2c3ccccc3c3ccccc32)nc(-c2c(-n3c4ccccc4c4ccccc43)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-n3c4ccccc4c4ccccc43)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3ccccc3c3ccccc32)nc(-c2c(-n3c4ccccc4c4ccccc43)cccc2-n2c3ccccc3c3ccccc32)n1]

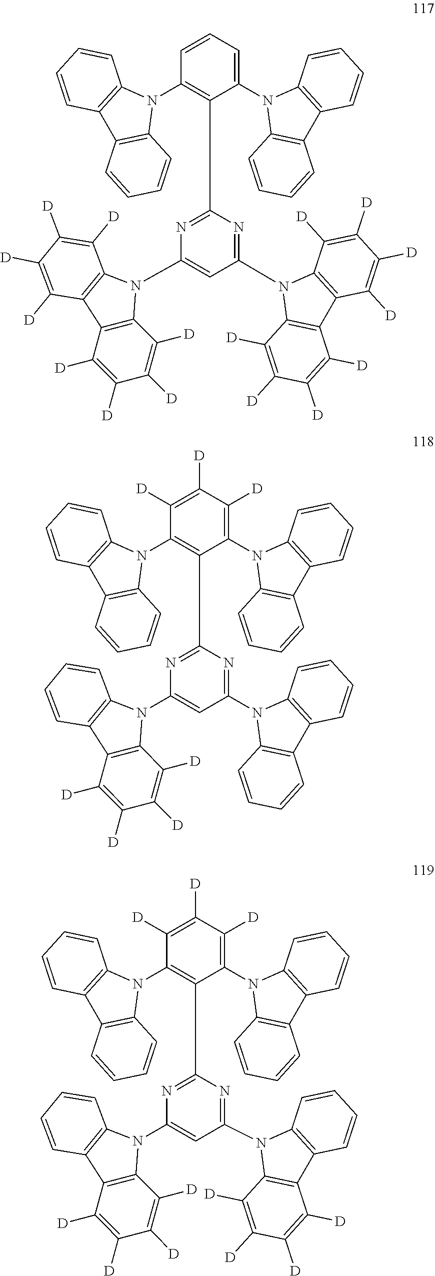

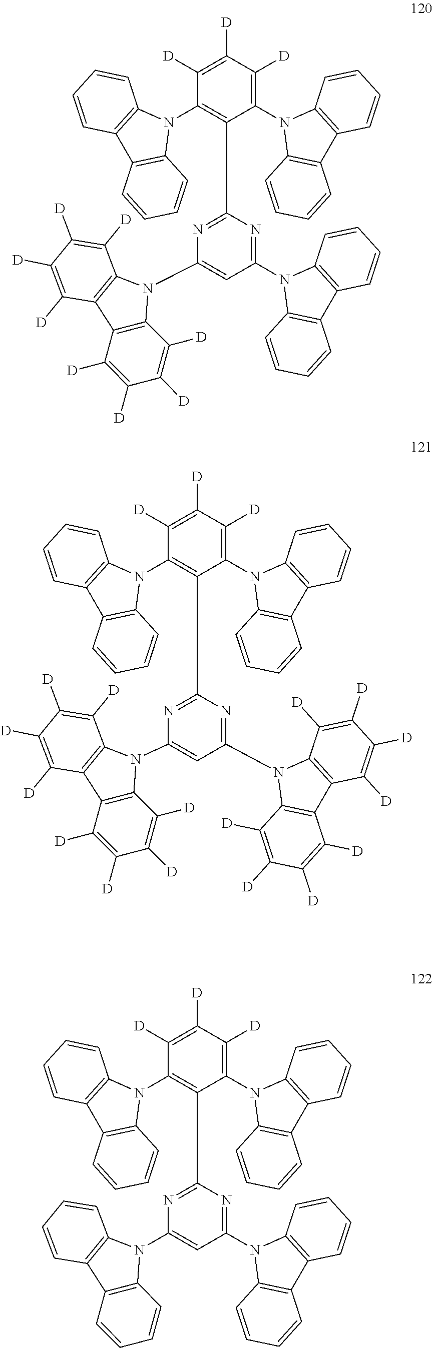

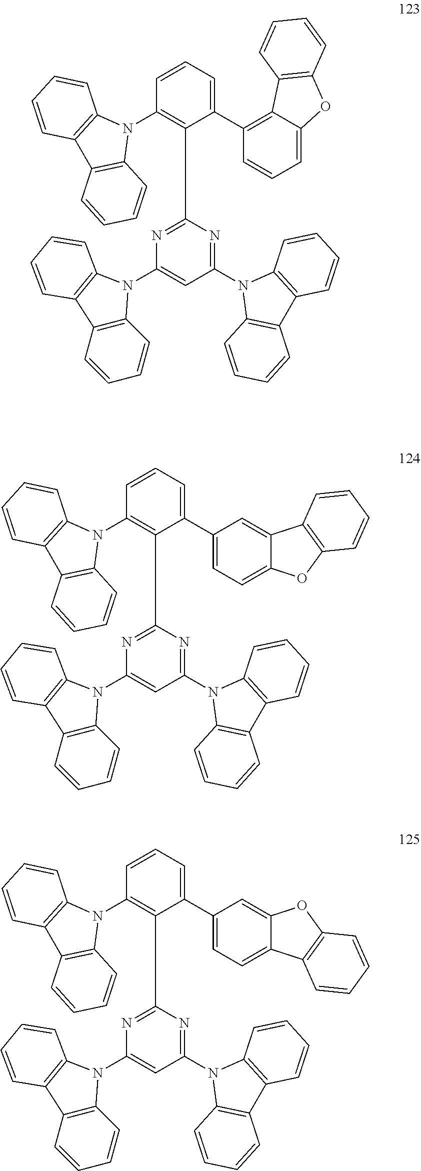 [SMILES: c1cc(-c2cccc3oc4ccccc4c23)c(-c2nc(-n3c4ccccc4c4ccccc43)cc(-n3c4ccccc4c4ccccc43)n2)c(-n2c3ccccc3c3ccccc32)c1 ; c1cc(-c2ccc3oc4ccccc4c3c2)c(-c2nc(-n3c4ccccc4c4ccccc43)cc(-n3c4ccccc4c4ccccc43)n2)c(-n2c3ccccc3c3ccccc32)c1 ; c1cc(-c2ccc3c(c2)oc2ccccc23)c(-c2nc(-n3c4ccccc4c4ccccc43)cc(-n3c4ccccc4c4ccccc43)n2)c(-n2c3ccccc3c3ccccc32)c1]

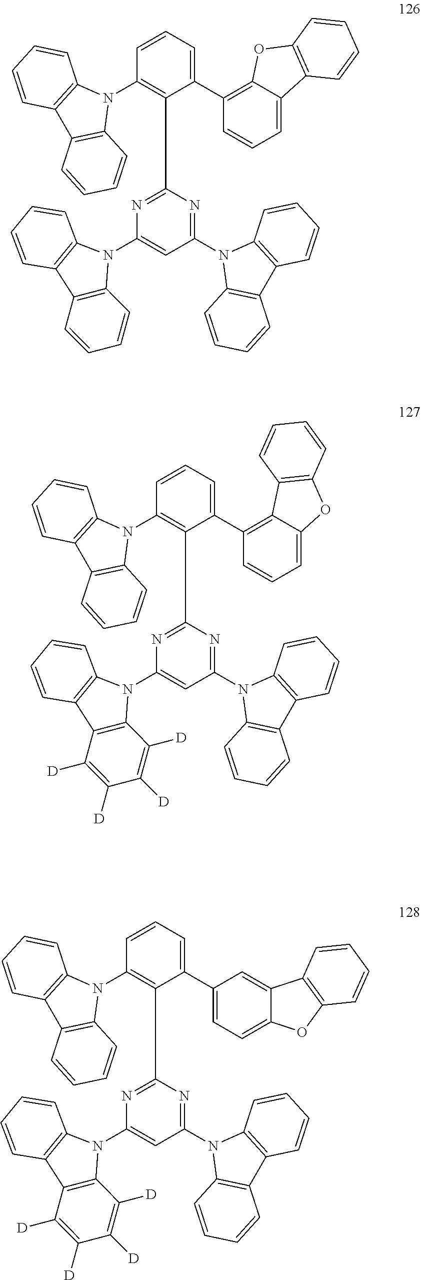 [SMILES: c1cc(-c2cccc3c2oc2ccccc23)c(-c2nc(-n3c4ccccc4c4ccccc43)cc(-n3c4ccccc4c4ccccc43)n2)c(-n2c3ccccc3c3ccccc32)c1 ; [2H]c1c([2H])c([2H])c2c(c1[2H])c1ccccc1n2-c1cc(-n2c3ccccc3c3ccccc32)nc(-c2c(-c3cccc4oc5ccccc5c34)cccc2-n2c3ccccc3c3ccccc32)n1 ; [2H]c1c([2H])c([2H])c2c(c1[2H])c1ccccc1n2-c1cc(-n2c3ccccc3c3ccccc32)nc(-c2c(-c3ccc4oc5ccccc5c4c3)cccc2-n2c3ccccc3c3ccccc32)n1]

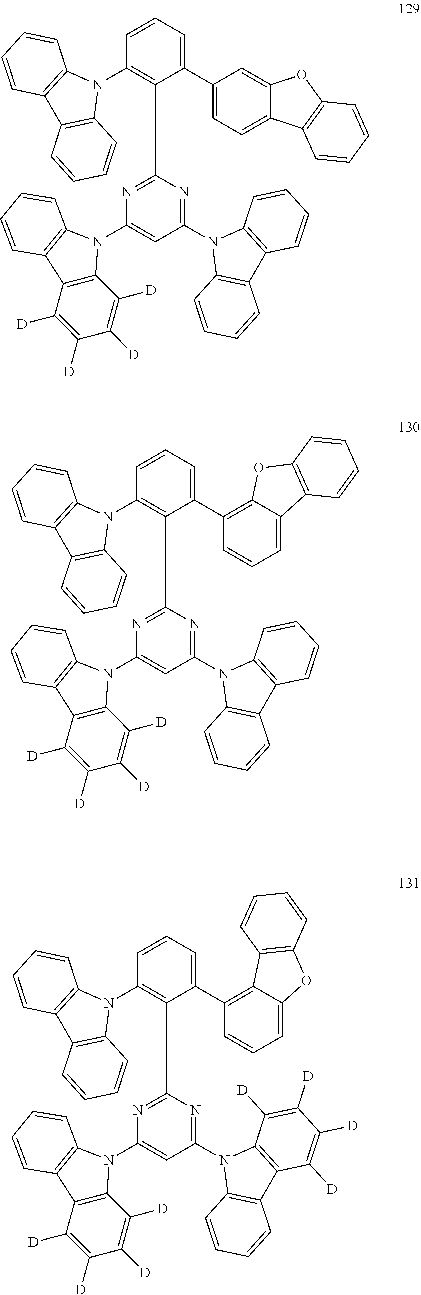 [SMILES: [2H]c1c([2H])c([2H])c2c(c1[2H])c1ccccc1n2-c1cc(-n2c3ccccc3c3ccccc32)nc(-c2c(-c3ccc4c(c3)oc3ccccc34)cccc2-n2c3ccccc3c3ccccc32)n1 ; [2H]c1c([2H])c([2H])c2c(c1[2H])c1ccccc1n2-c1cc(-n2c3ccccc3c3ccccc32)nc(-c2c(-c3cccc4c3oc3ccccc34)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-c3cccc4oc5ccccc5c34)cccc2-n2c3ccccc3c3ccccc32)n1]

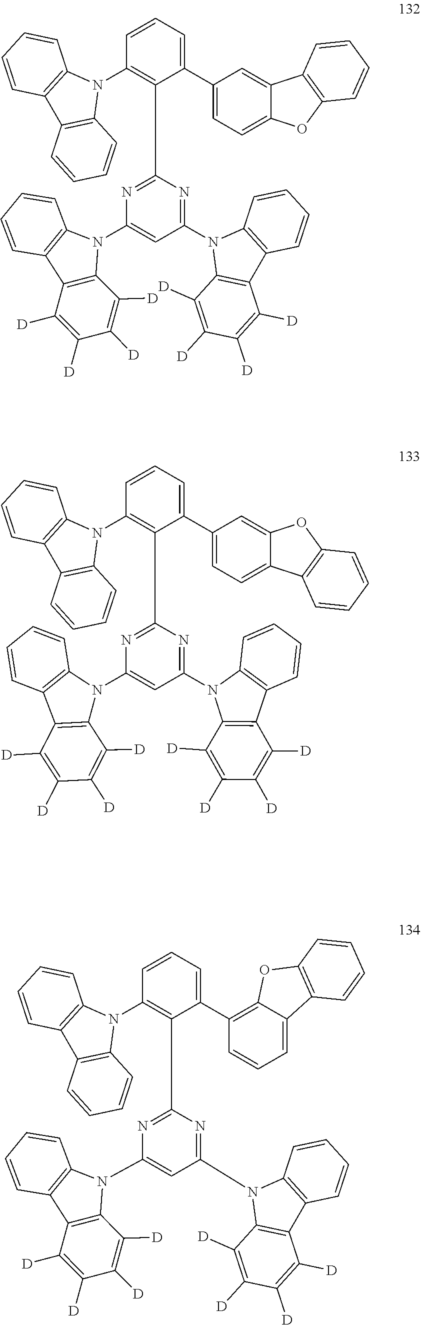 [SMILES: [2H]c1c([2H])c([2H])c2c(c1[2H])c1ccccc1n2-c1cc(-n2c3ccccc3c3c([2H])c([2H])c([2H])c([2H])c32)nc(-c2c(-c3ccc4oc5ccccc5c4c3)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-c3ccc4c(c3)oc3ccccc34)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-c3cccc4c3oc3ccccc34)cccc2-n2c3ccccc3c3ccccc32)n1]

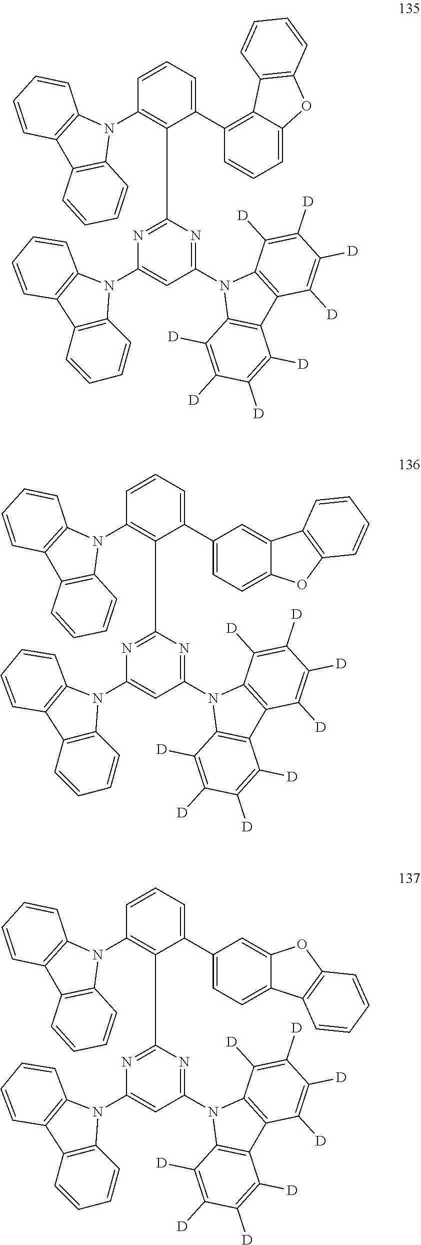 [SMILES: [2H]c1c([2H])c([2H])c2c(c1[2H])c1c([2H])c([2H])c([2H])c([2H])c1n2-c1cc(-n2c3ccccc3c3ccccc32)nc(-c2c(-c3cccc4oc5ccccc5c34)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3ccccc3c3ccccc32)nc(-c2c(-c3ccc4oc5ccccc5c4c3)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3ccccc3c3ccccc32)nc(-c2c(-c3ccc4c(c3)oc3ccccc34)cccc2-n2c3ccccc3c3ccccc32)n1]

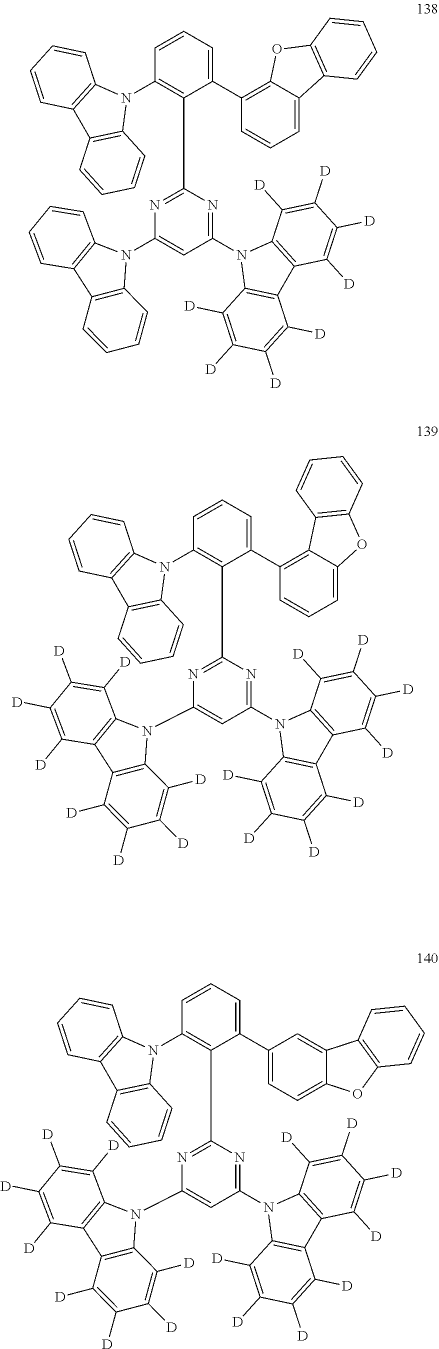

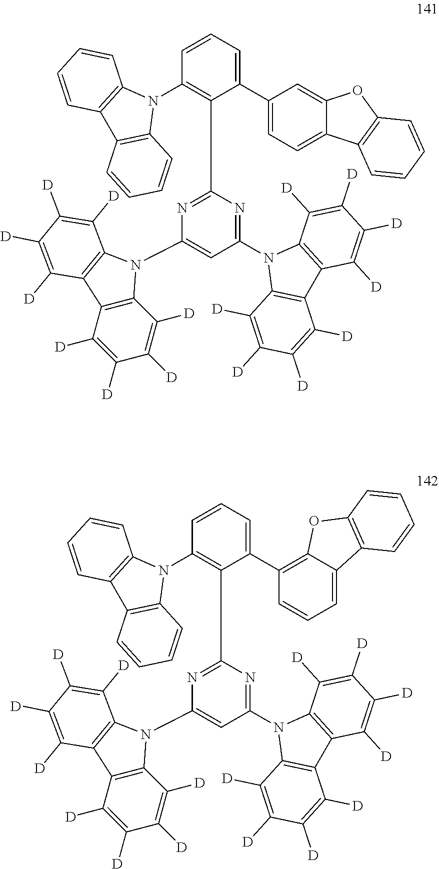 [SMILES: [2H]c1c([2H])c([2H])c2c(c1[2H])c1c([2H])c([2H])c([2H])c([2H])c1n2-c1cc(-n2c3c([2H])c([2H])c([2H])c([2H])c3c3c([2H])c([2H])c([2H])c([2H])c32)nc(-c2c(-c3ccc4c(c3)oc3ccccc34)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3c([2H])c([2H])c([2H])c([2H])c3c3c([2H])c([2H])c([2H])c([2H])c32)nc(-c2c(-c3cccc4c3oc3ccccc34)cccc2-n2c3ccccc3c3ccccc32)n1]

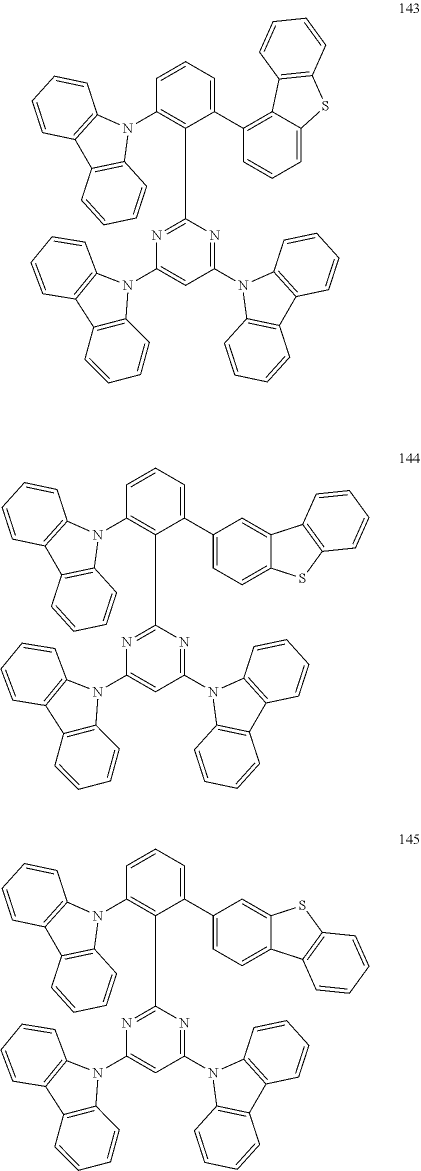 [SMILES: c1cc(-c2cccc3sc4ccccc4c23)c(-c2nc(-n3c4ccccc4c4ccccc43)cc(-n3c4ccccc4c4ccccc43)n2)c(-n2c3ccccc3c3ccccc32)c1 ; c1cc(-c2ccc3sc4ccccc4c3c2)c(-c2nc(-n3c4ccccc4c4ccccc43)cc(-n3c4ccccc4c4ccccc43)n2)c(-n2c3ccccc3c3ccccc32)c1 ; c1cc(-c2ccc3c(c2)sc2ccccc23)c(-c2nc(-n3c4ccccc4c4ccccc43)cc(-n3c4ccccc4c4ccccc43)n2)c(-n2c3ccccc3c3ccccc32)c1]

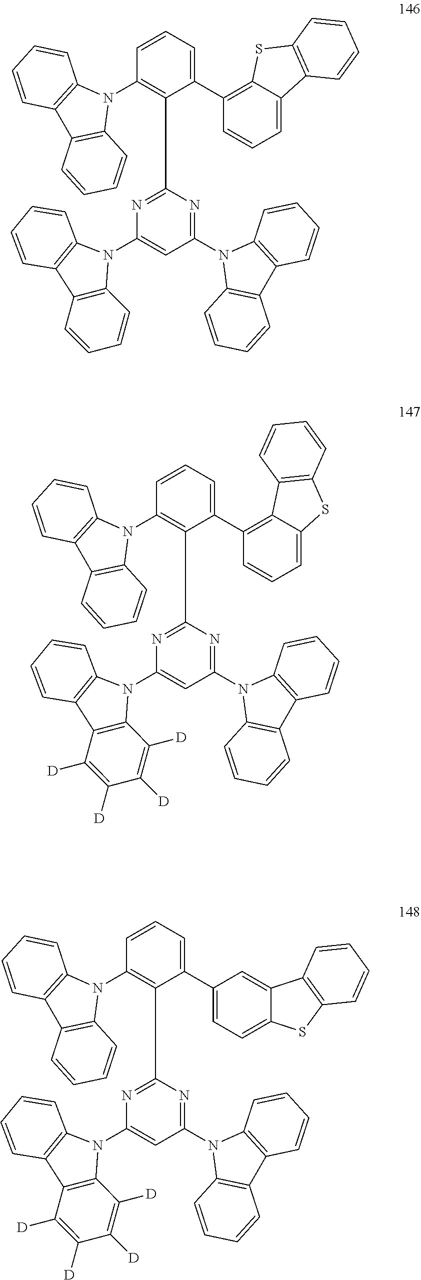 [SMILES: c1cc(-c2cccc3c2sc2ccccc23)c(-c2nc(-n3c4ccccc4c4ccccc43)cc(-n3c4ccccc4c4ccccc43)n2)c(-n2c3ccccc3c3ccccc32)c1 ; [2H]c1c([2H])c([2H])c2c(c1[2H])c1ccccc1n2-c1cc(-n2c3ccccc3c3ccccc32)nc(-c2c(-c3cccc4sc5ccccc5c34)cccc2-n2c3ccccc3c3ccccc32)n1 ; [2H]c1c([2H])c([2H])c2c(c1[2H])c1ccccc1n2-c1cc(-n2c3ccccc3c3ccccc32)nc(-c2c(-c3ccc4sc5ccccc5c4c3)cccc2-n2c3ccccc3c3ccccc32)n1]

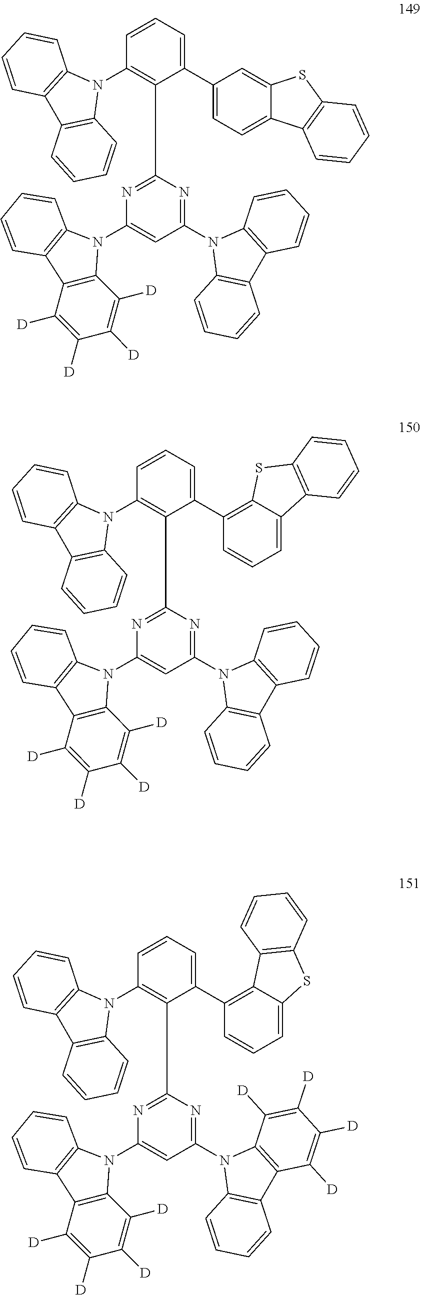 [SMILES: [2H]c1c([2H])c([2H])c2c(c1[2H])c1ccccc1n2-c1cc(-n2c3ccccc3c3ccccc32)nc(-c2c(-c3ccc4c(c3)sc3ccccc34)cccc2-n2c3ccccc3c3ccccc32)n1 ; [2H]c1c([2H])c([2H])c2c(c1[2H])c1ccccc1n2-c1cc(-n2c3ccccc3c3ccccc32)nc(-c2c(-c3cccc4c3sc3ccccc34)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-c3cccc4sc5ccccc5c34)cccc2-n2c3ccccc3c3ccccc32)n1]

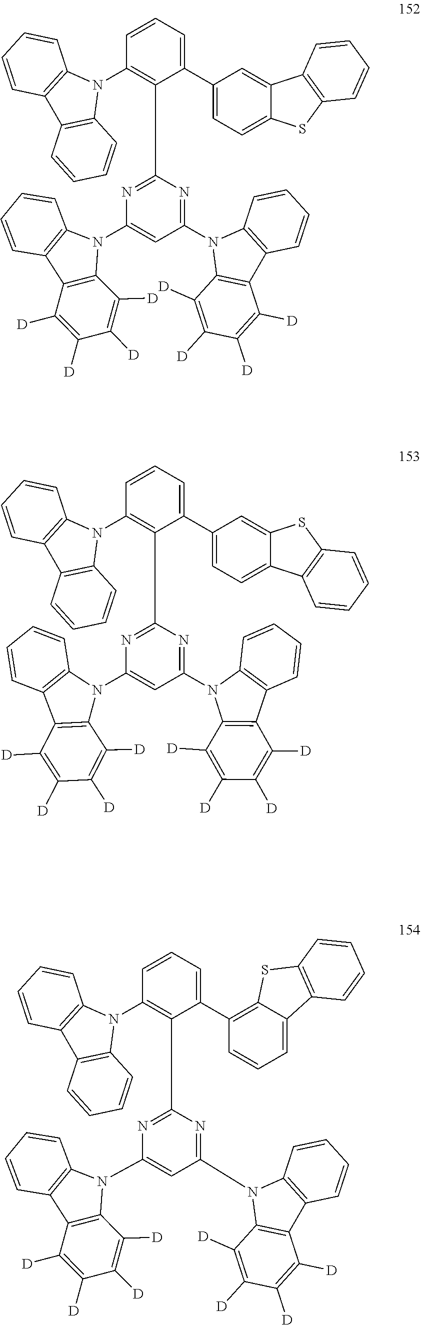 [SMILES: [2H]c1c([2H])c([2H])c2c(c1[2H])c1ccccc1n2-c1cc(-n2c3ccccc3c3c([2H])c([2H])c([2H])c([2H])c32)nc(-c2c(-c3ccc4sc5ccccc5c4c3)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-c3ccc4c(c3)sc3ccccc34)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-c3cccc4c3sc3ccccc34)cccc2-n2c3ccccc3c3ccccc32)n1]

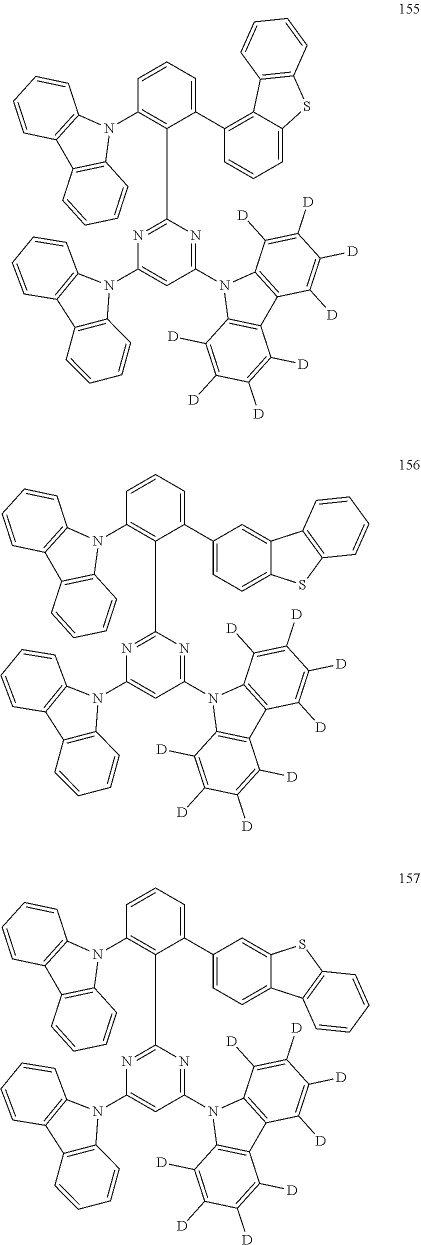 [SMILES: [2H]c1c([2H])c([2H])c2c(c1[2H])c1c([2H])c([2H])c([2H])c([2H])c1n2-c1cc(-n2c3ccccc3c3ccccc32)nc(-c2c(-c3cccc4sc5ccccc5c34)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3ccccc3c3ccccc32)nc(-c2c(-c3ccc4sc5ccccc5c4c3)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3ccccc3c3ccccc32)nc(-c2c(-c3ccc4c(c3)sc3ccccc34)cccc2-n2c3ccccc3c3ccccc32)n1]

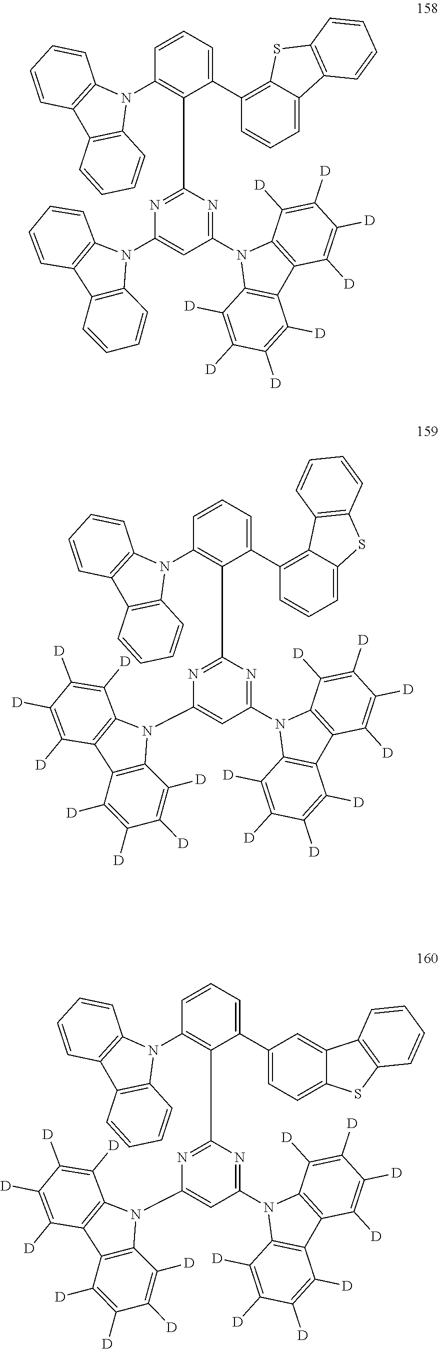

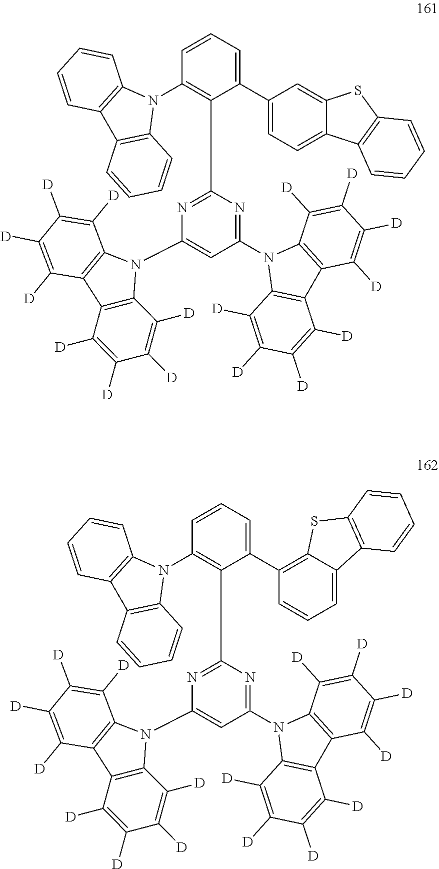 [SMILES: [2H]c1c([2H])c([2H])c2c(c1[2H])c1c([2H])c([2H])c([2H])c([2H])c1n2-c1cc(-n2c3c([2H])c([2H])c([2H])c([2H])c3c3c([2H])c([2H])c([2H])c([2H])c32)nc(-c2c(-c3ccc4c(c3)sc3ccccc34)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3c([2H])c([2H])c([2H])c([2H])c3c3c([2H])c([2H])c([2H])c([2H])c32)nc(-c2c(-c3cccc4c3sc3ccccc34)cccc2-n2c3ccccc3c3ccccc32)n1]

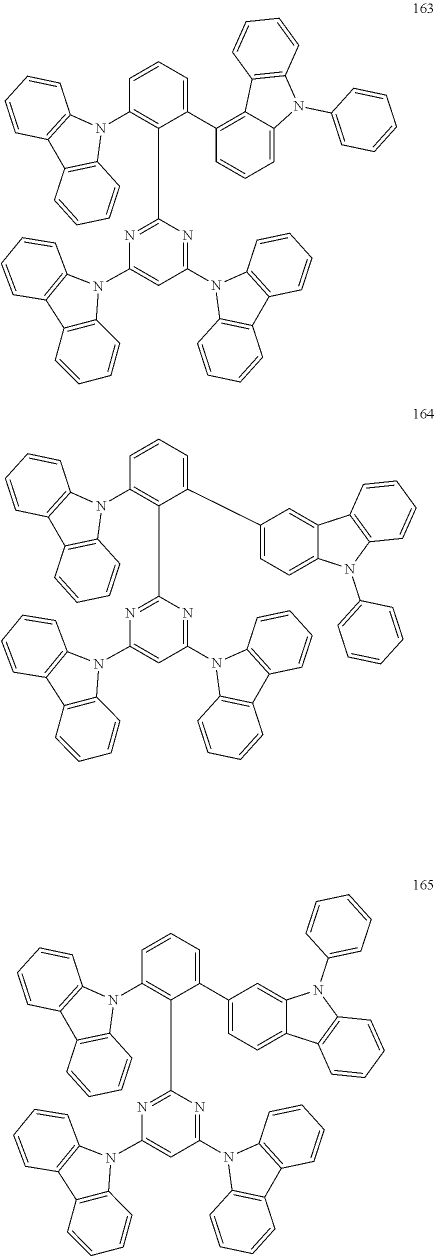 [SMILES: c1ccc(-n2c3ccccc3c3c(-c4cccc(-n5c6ccccc6c6ccccc65)c4-c4nc(-n5c6ccccc6c6ccccc65)cc(-n5c6ccccc6c6ccccc65)n4)cccc32)cc1 ; c1ccc(-n2c3ccccc3c3cc(-c4cccc(-n5c6ccccc6c6ccccc65)c4-c4nc(-n5c6ccccc6c6ccccc65)cc(-n5c6ccccc6c6ccccc65)n4)ccc32)cc1 ; c1ccc(-n2c3ccccc3c3ccc(-c4cccc(-n5c6ccccc6c6ccccc65)c4-c4nc(-n5c6ccccc6c6ccccc65)cc(-n5c6ccccc6c6ccccc65)n4)cc32)cc1]

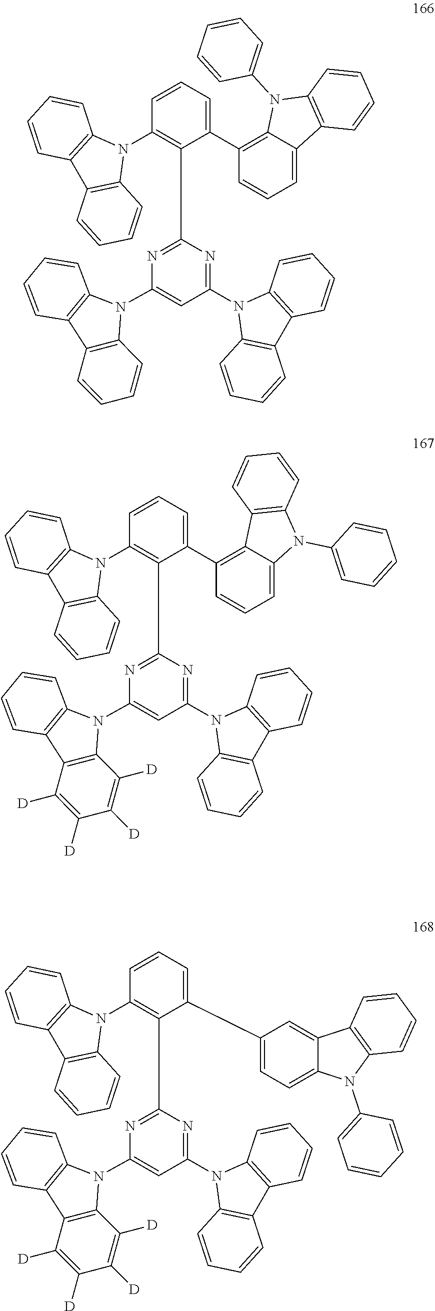 [SMILES: c1ccc(-n2c3ccccc3c3cccc(-c4cccc(-n5c6ccccc6c6ccccc65)c4-c4nc(-n5c6ccccc6c6ccccc65)cc(-n5c6ccccc6c6ccccc65)n4)c32)cc1 ; [2H]c1c([2H])c([2H])c2c(c1[2H])c1ccccc1n2-c1cc(-n2c3ccccc3c3ccccc32)nc(-c2c(-c3cccc4c3c3ccccc3n4-c3ccccc3)cccc2-n2c3ccccc3c3ccccc32)n1 ; [2H]c1c([2H])c([2H])c2c(c1[2H])c1ccccc1n2-c1cc(-n2c3ccccc3c3ccccc32)nc(-c2c(-c3ccc4c(c3)c3ccccc3n4-c3ccccc3)cccc2-n2c3ccccc3c3ccccc32)n1]

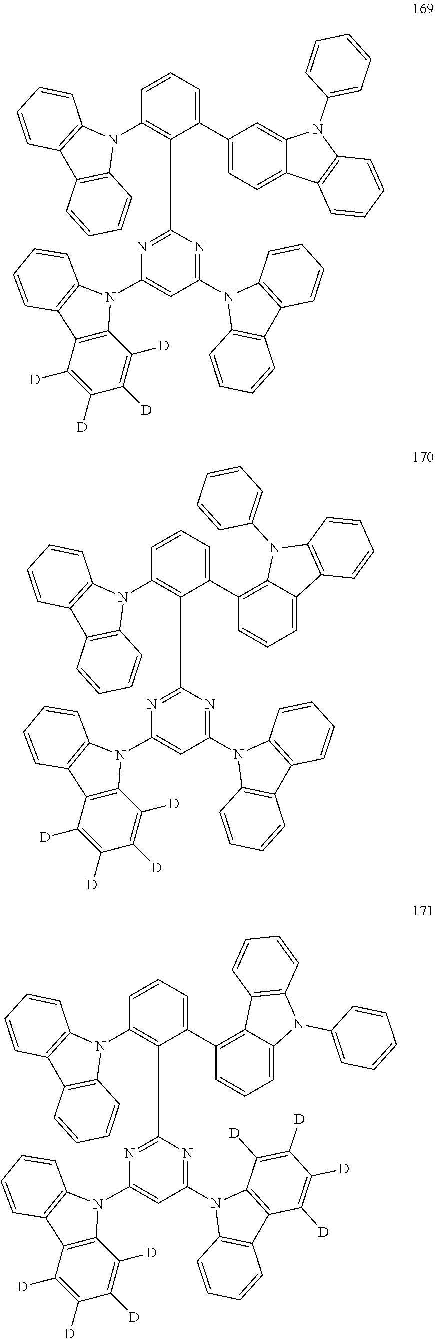 [SMILES: [2H]c1c([2H])c([2H])c2c(c1[2H])c1ccccc1n2-c1cc(-n2c3ccccc3c3ccccc32)nc(-c2c(-c3ccc4c5ccccc5n(-c5ccccc5)c4c3)cccc2-n2c3ccccc3c3ccccc32)n1 ; [2H]c1c([2H])c([2H])c2c(c1[2H])c1ccccc1n2-c1cc(-n2c3ccccc3c3ccccc32)nc(-c2c(-c3cccc4c5ccccc5n(-c5ccccc5)c34)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-c3cccc4c3c3ccccc3n4-c3ccccc3)cccc2-n2c3ccccc3c3ccccc32)n1]

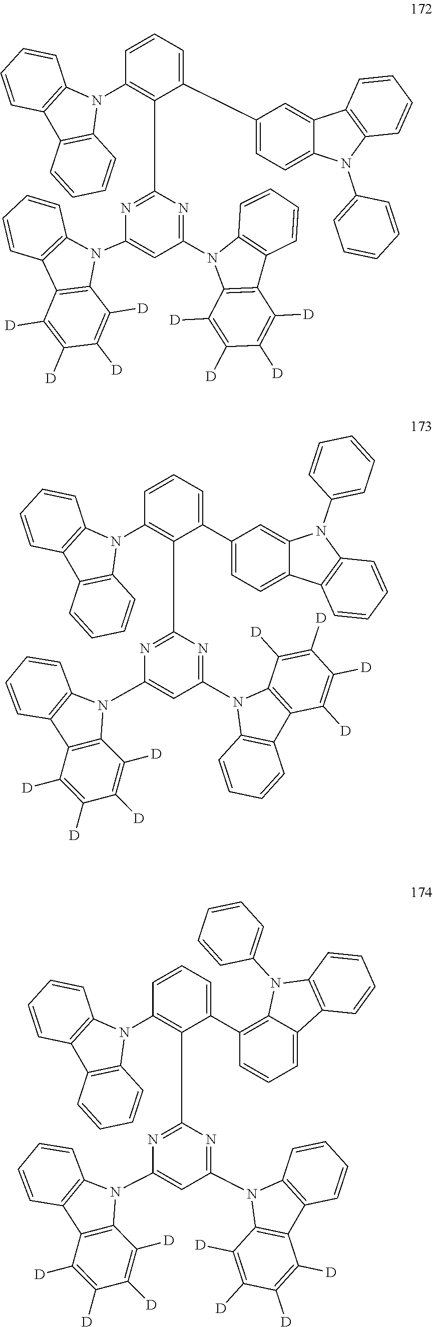 [SMILES: [2H]c1c([2H])c([2H])c2c(c1[2H])c1ccccc1n2-c1cc(-n2c3ccccc3c3c([2H])c([2H])c([2H])c([2H])c32)nc(-c2c(-c3ccc4c(c3)c3ccccc3n4-c3ccccc3)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-c3ccc4c5ccccc5n(-c5ccccc5)c4c3)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-c3cccc4c5ccccc5n(-c5ccccc5)c34)cccc2-n2c3ccccc3c3ccccc32)n1]

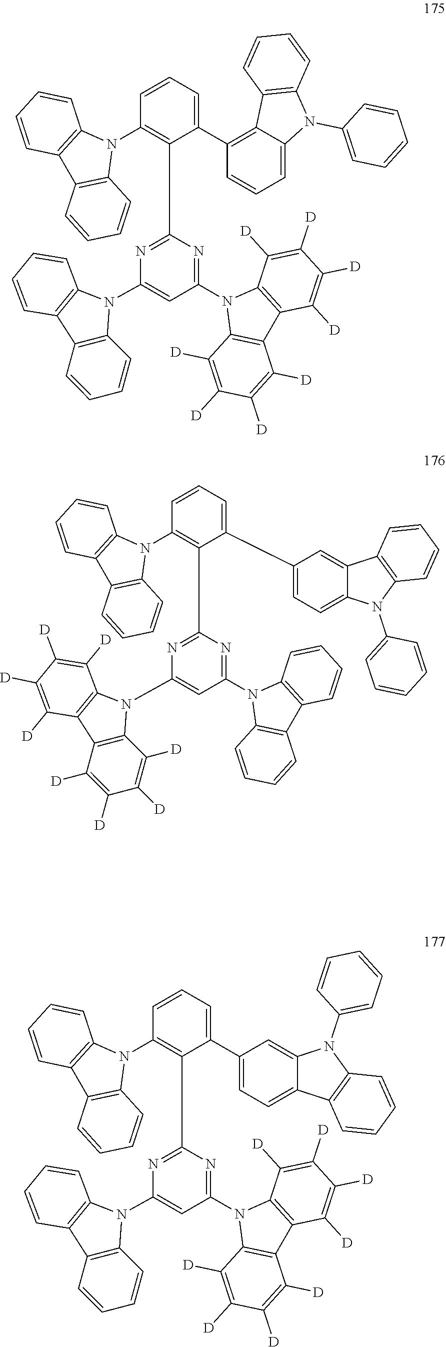 [SMILES: [2H]c1c([2H])c([2H])c2c(c1[2H])c1c([2H])c([2H])c([2H])c([2H])c1n2-c1cc(-n2c3ccccc3c3ccccc32)nc(-c2c(-c3cccc4c3c3ccccc3n4-c3ccccc3)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3ccccc3c3ccccc32)nc(-c2c(-c3ccc4c(c3)c3ccccc3n4-c3ccccc3)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3ccccc3c3ccccc32)nc(-c2c(-c3ccc4c5ccccc5n(-c5ccccc5)c4c3)cccc2-n2c3ccccc3c3ccccc32)n1]

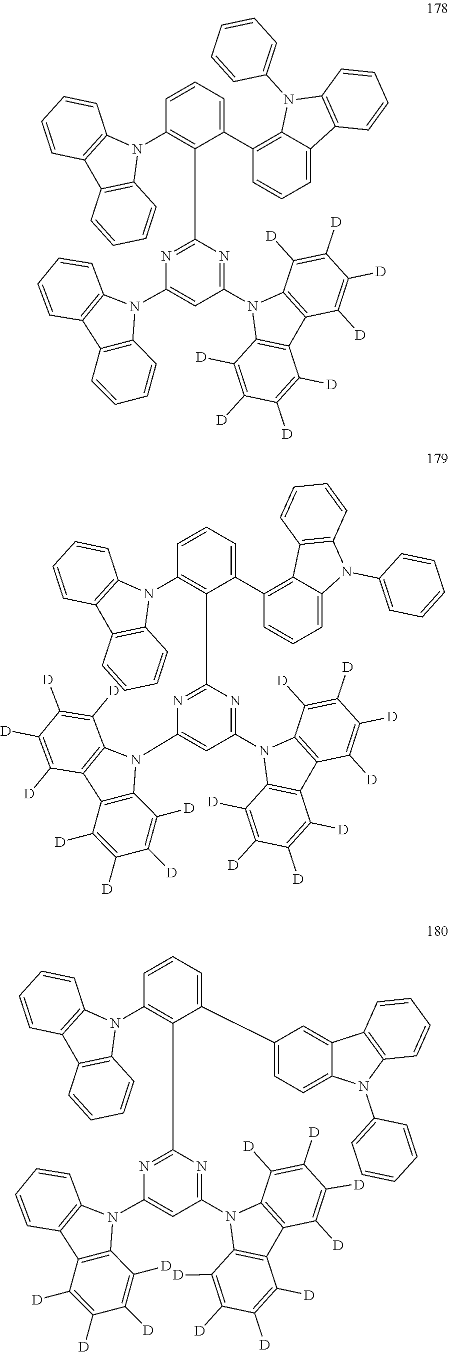

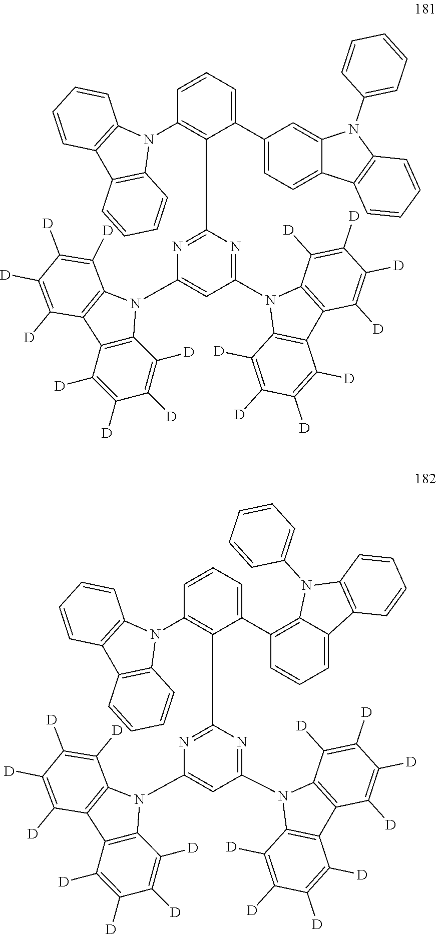 [SMILES: [2H]c1c([2H])c([2H])c2c(c1[2H])c1c([2H])c([2H])c([2H])c([2H])c1n2-c1cc(-n2c3c([2H])c([2H])c([2H])c([2H])c3c3c([2H])c([2H])c([2H])c([2H])c32)nc(-c2c(-c3ccc4c5ccccc5n(-c5ccccc5)c4c3)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3c([2H])c([2H])c([2H])c([2H])c3c3c([2H])c([2H])c([2H])c([2H])c32)nc(-c2c(-c3cccc4c5ccccc5n(-c5ccccc5)c34)cccc2-n2c3ccccc3c3ccccc32)n1]

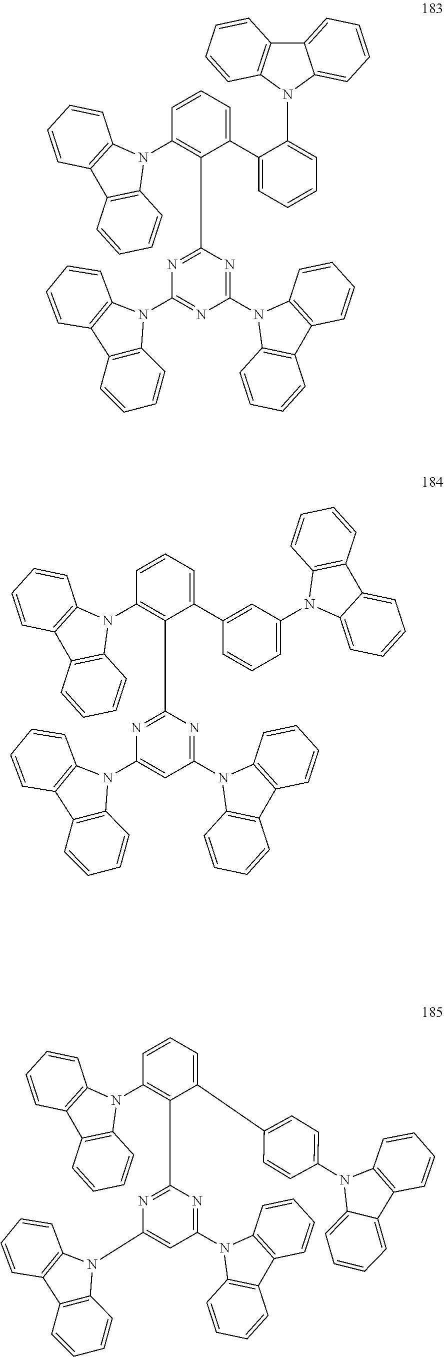 [SMILES: c1ccc(-n2c3ccccc3c3ccccc32)c(-c2cccc(-n3c4ccccc4c4ccccc43)c2-c2nc(-n3c4ccccc4c4ccccc43)nc(-n3c4ccccc4c4ccccc43)n2)c1 ; c1cc(-c2cccc(-n3c4ccccc4c4ccccc43)c2-c2nc(-n3c4ccccc4c4ccccc43)cc(-n3c4ccccc4c4ccccc43)n2)cc(-n2c3ccccc3c3ccccc32)c1 ; c1cc(-c2ccc(-n3c4ccccc4c4ccccc43)cc2)c(-c2nc(-n3c4ccccc4c4ccccc43)cc(-n3c4ccccc4c4ccccc43)n2)c(-n2c3ccccc3c3ccccc32)c1]

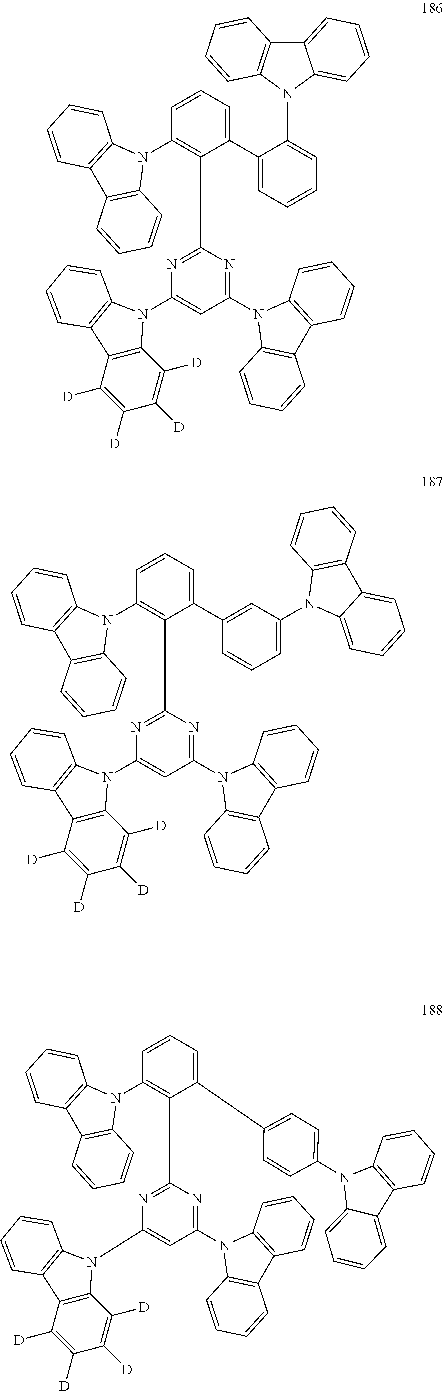 [SMILES: [2H]c1c([2H])c([2H])c2c(c1[2H])c1ccccc1n2-c1cc(-n2c3ccccc3c3ccccc32)nc(-c2c(-c3ccccc3-n3c4ccccc4c4ccccc43)cccc2-n2c3ccccc3c3ccccc32)n1 ; [2H]c1c([2H])c([2H])c2c(c1[2H])c1ccccc1n2-c1cc(-n2c3ccccc3c3ccccc32)nc(-c2c(-c3cccc(-n4c5ccccc5c5ccccc54)c3)cccc2-n2c3ccccc3c3ccccc32)n1 ; [2H]c1c([2H])c([2H])c2c(c1[2H])c1ccccc1n2-c1cc(-n2c3ccccc3c3ccccc32)nc(-c2c(-c3ccc(-n4c5ccccc5c5ccccc54)cc3)cccc2-n2c3ccccc3c3ccccc32)n1]

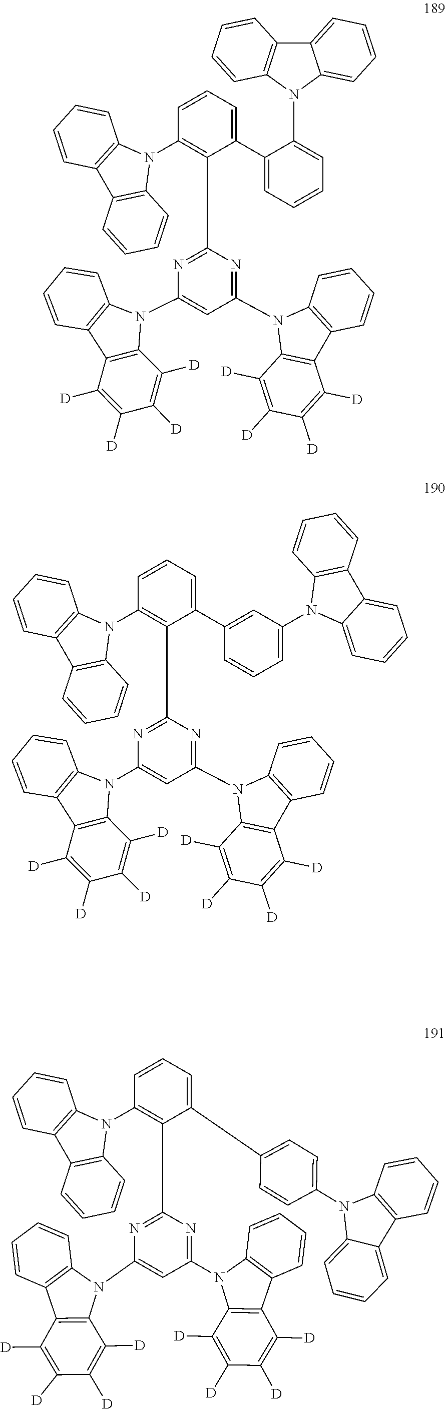 [SMILES: [2H]c1c([2H])c([2H])c2c(c1[2H])c1ccccc1n2-c1cc(-n2c3ccccc3c3c([2H])c([2H])c([2H])c([2H])c32)nc(-c2c(-c3ccccc3-n3c4ccccc4c4ccccc43)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-c3cccc(-n4c5ccccc5c5ccccc54)c3)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-c3ccc(-n4c5ccccc5c5ccccc54)cc3)cccc2-n2c3ccccc3c3ccccc32)n1]

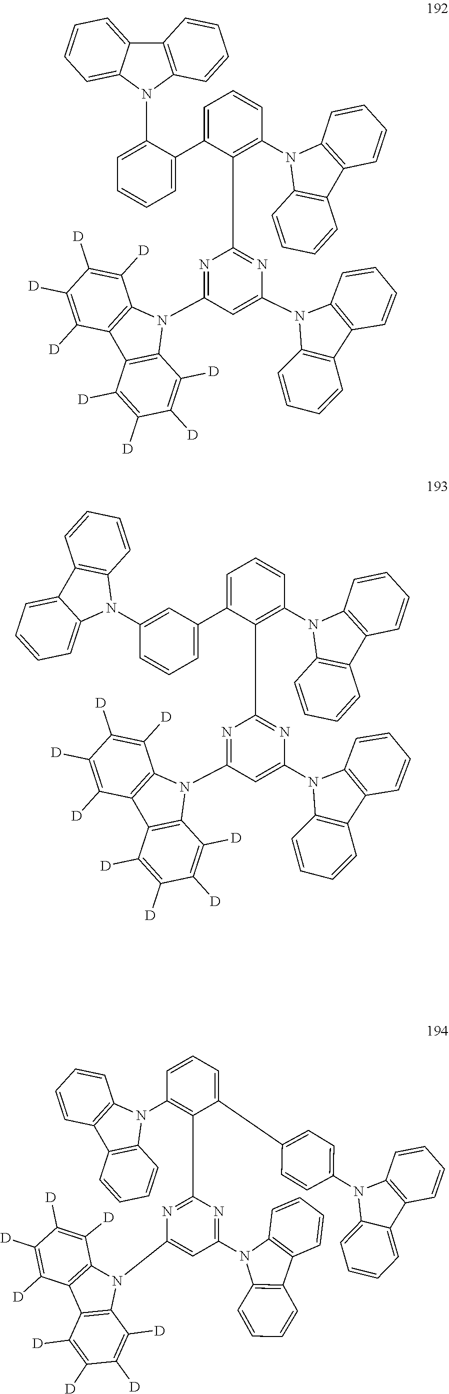 [SMILES: [2H]c1c([2H])c([2H])c2c(c1[2H])c1c([2H])c([2H])c([2H])c([2H])c1n2-c1cc(-n2c3ccccc3c3ccccc32)nc(-c2c(-c3ccccc3-n3c4ccccc4c4ccccc43)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3ccccc3c3ccccc32)nc(-c2c(-c3cccc(-n4c5ccccc5c5ccccc54)c3)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3ccccc3c3ccccc32)nc(-c2c(-c3ccc(-n4c5ccccc5c5ccccc54)cc3)cccc2-n2c3ccccc3c3ccccc32)n1]

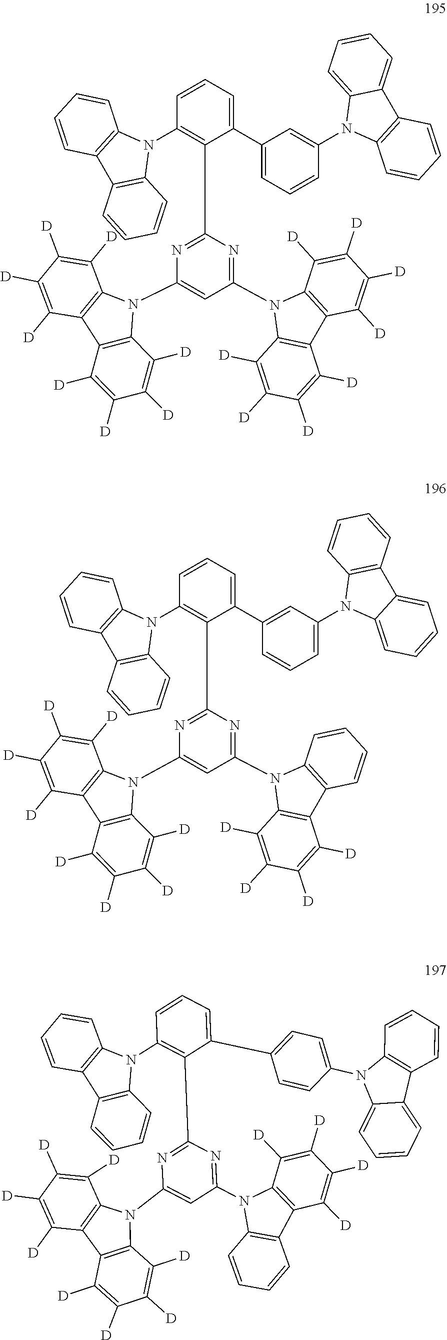

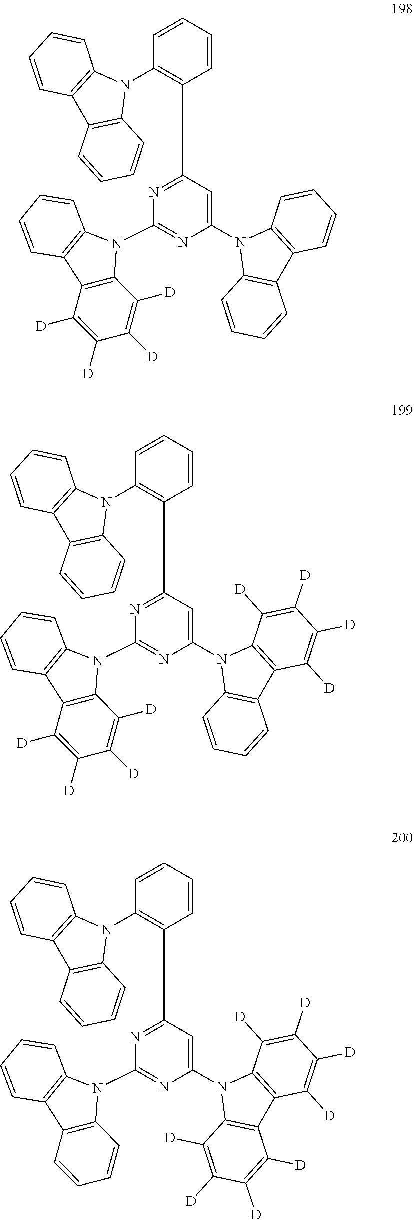 [SMILES: [2H]c1c([2H])c([2H])c2c(c1[2H])c1ccccc1n2-c1nc(-c2ccccc2-n2c3ccccc3c3ccccc32)cc(-n2c3ccccc3c3ccccc32)n1 ; [2H]c1c([2H])c([2H])c2c(c1[2H])c1ccccc1n2-c1cc(-c2ccccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1c([2H])c([2H])c([2H])c([2H])c1n2-c1cc(-c2ccccc2-n2c3ccccc3c3ccccc32)nc(-n2c3ccccc3c3ccccc32)n1]

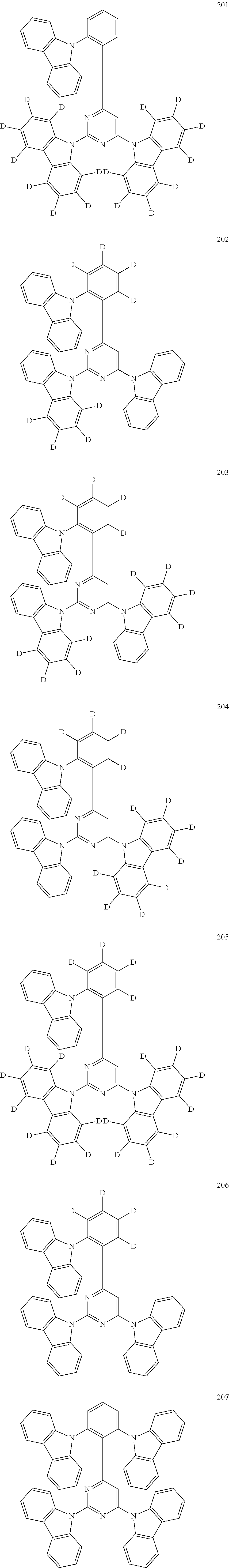

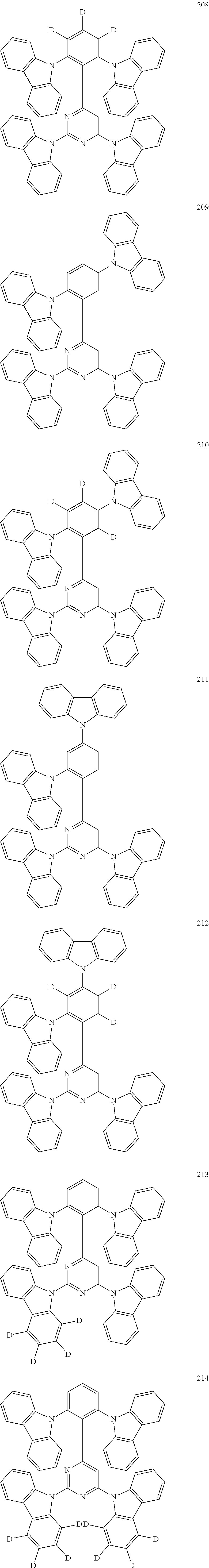

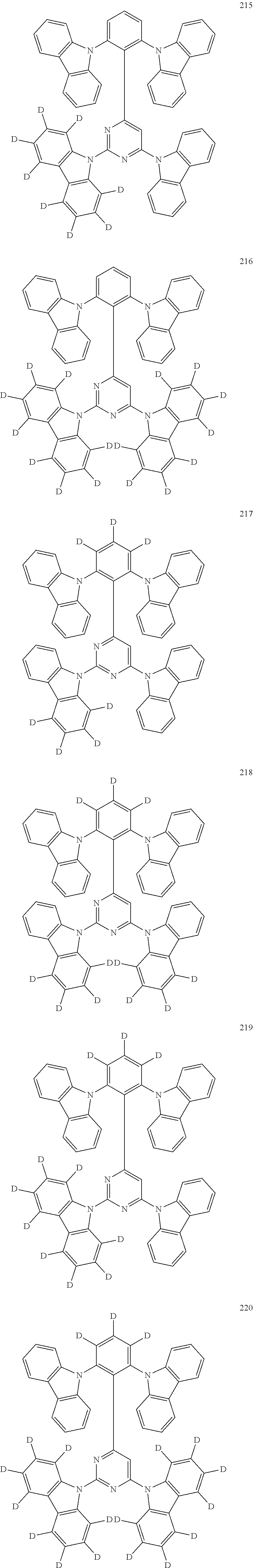

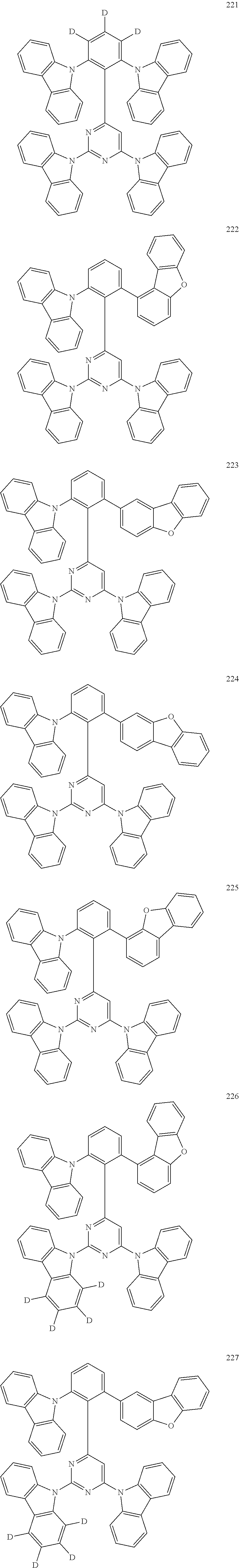

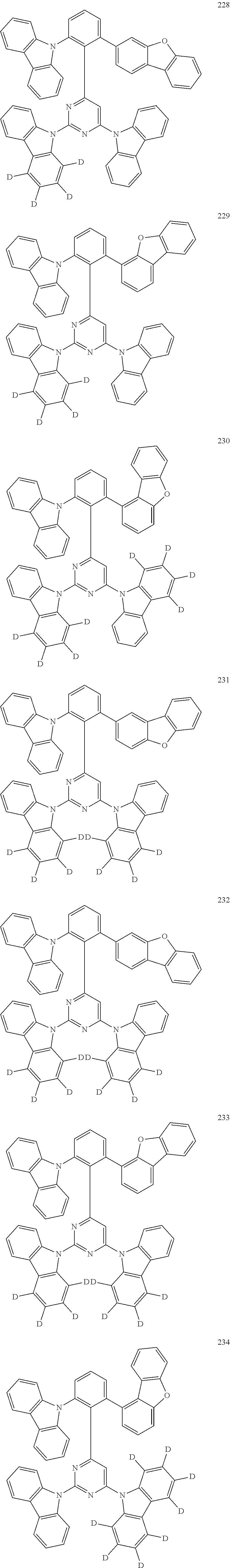

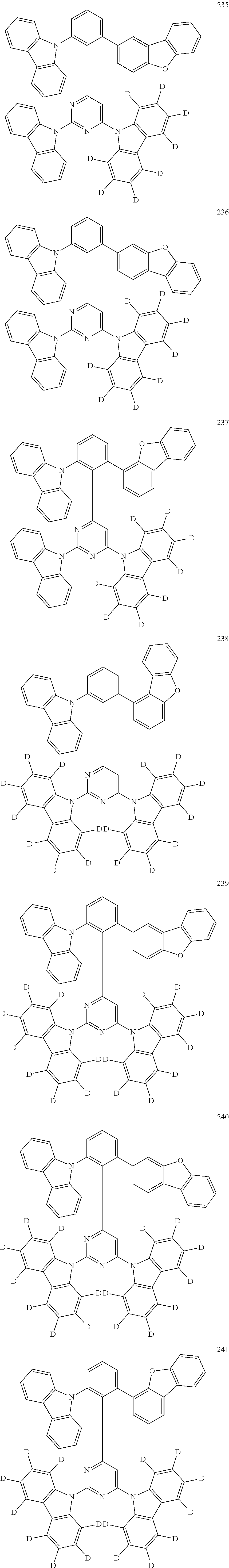

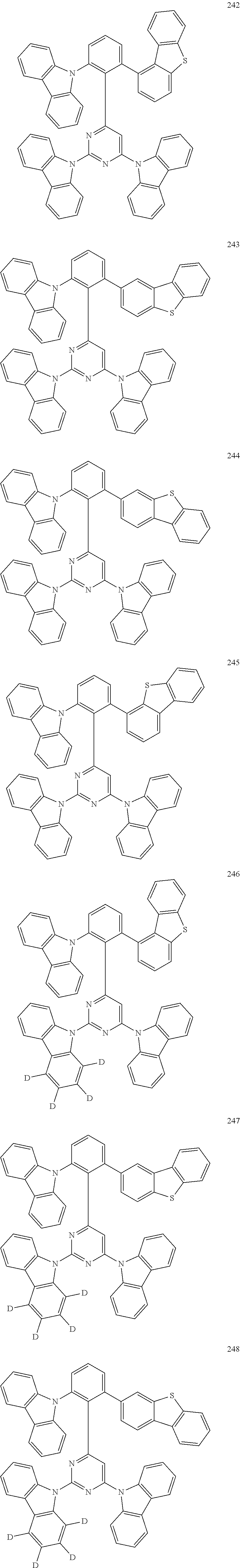

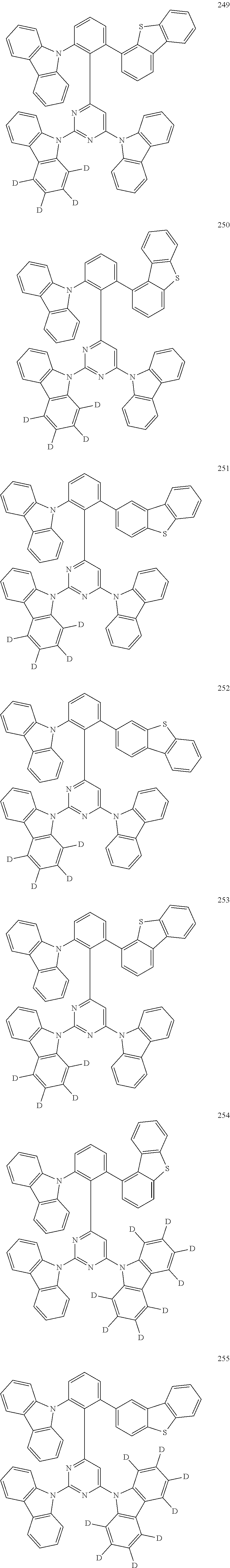

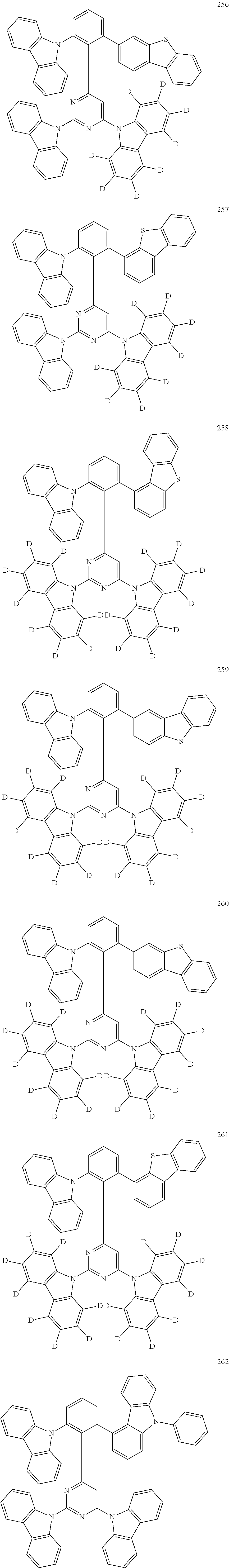

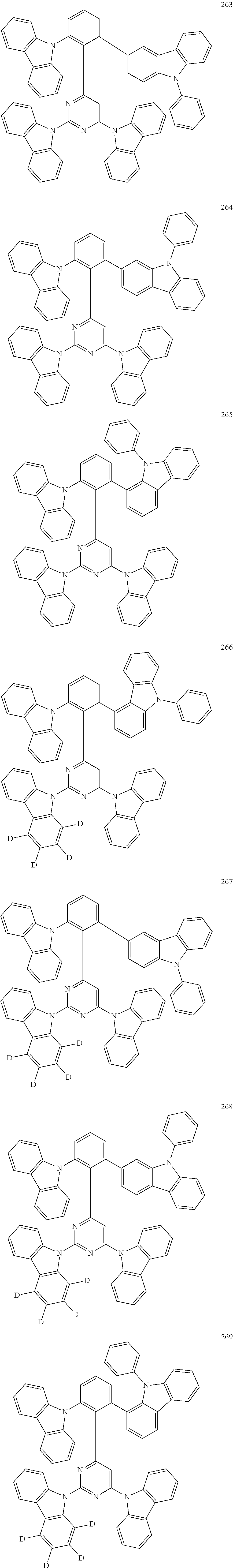

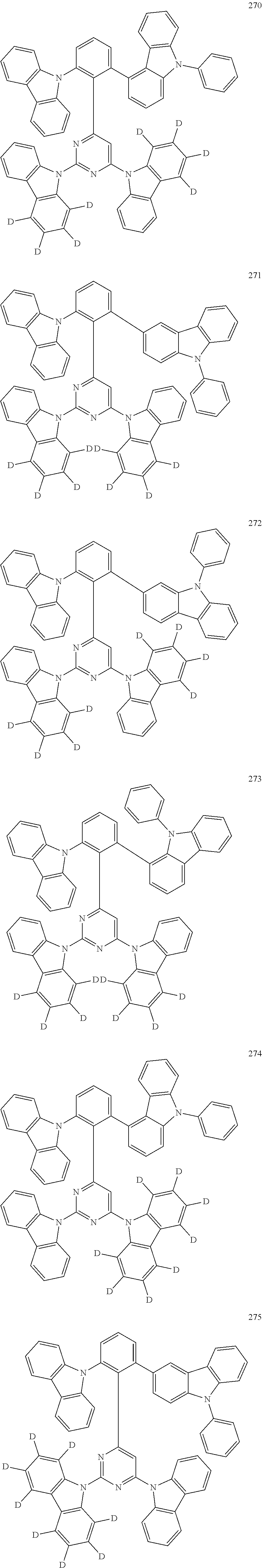

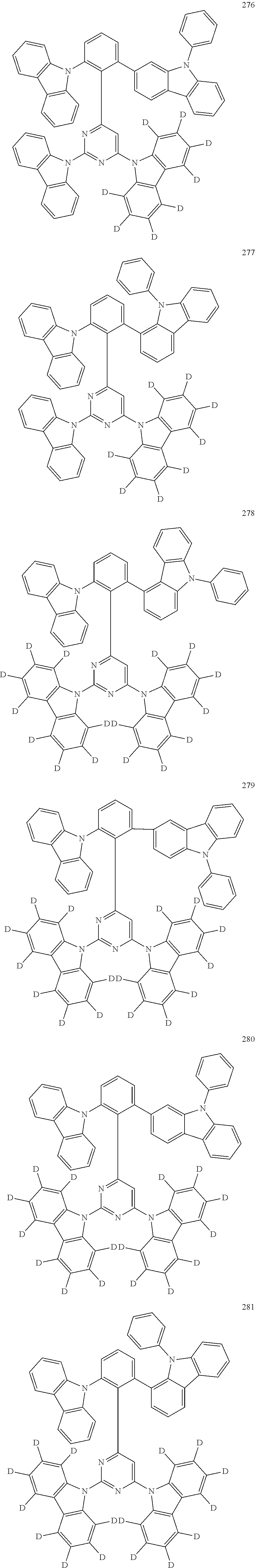

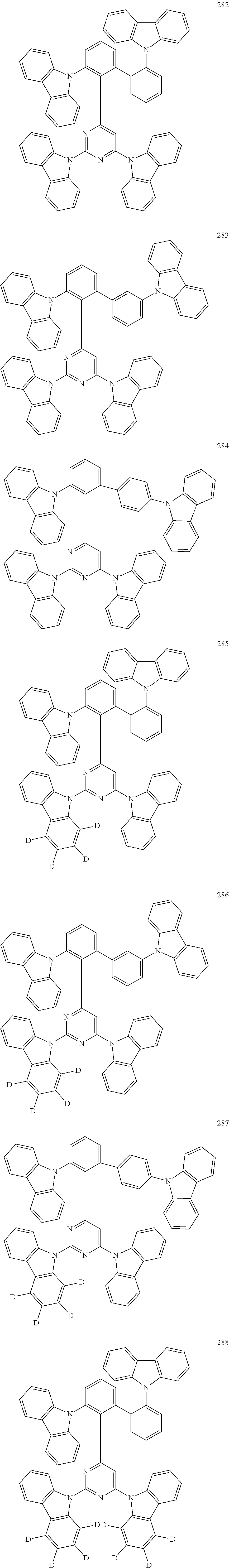

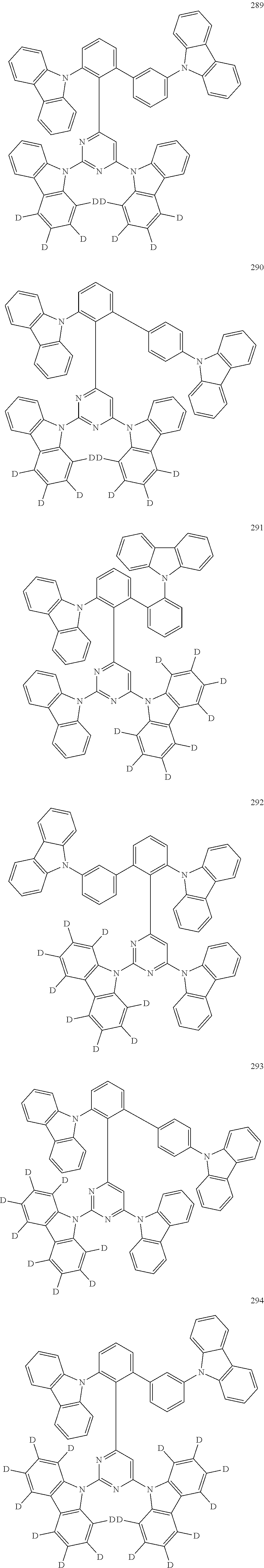

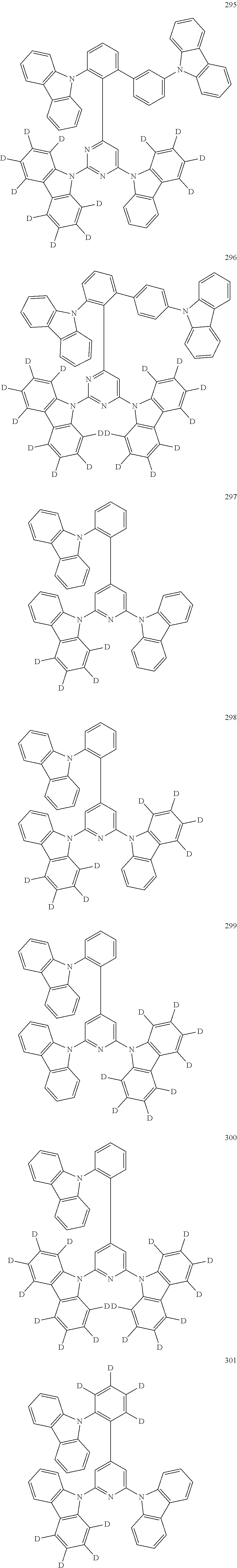

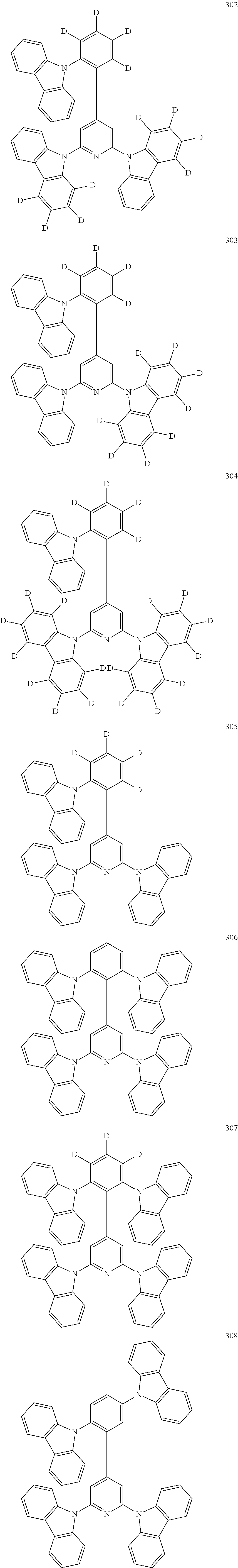

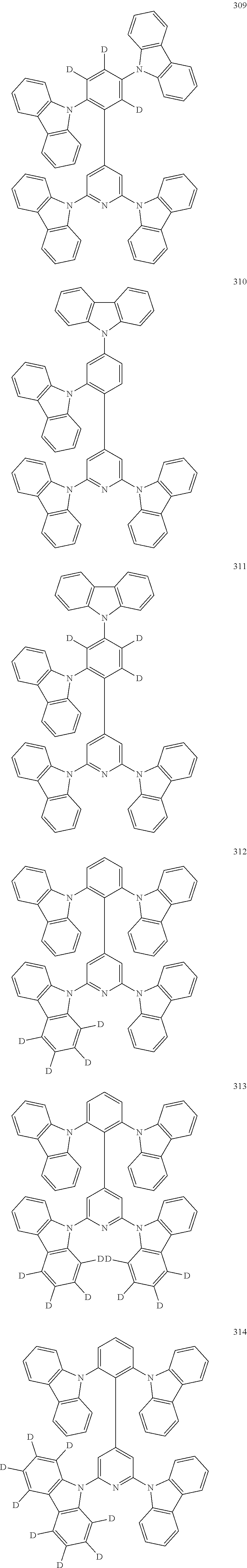

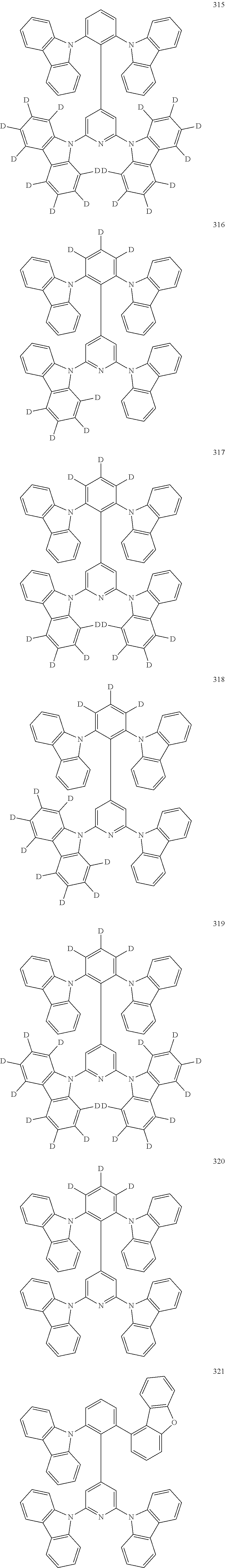

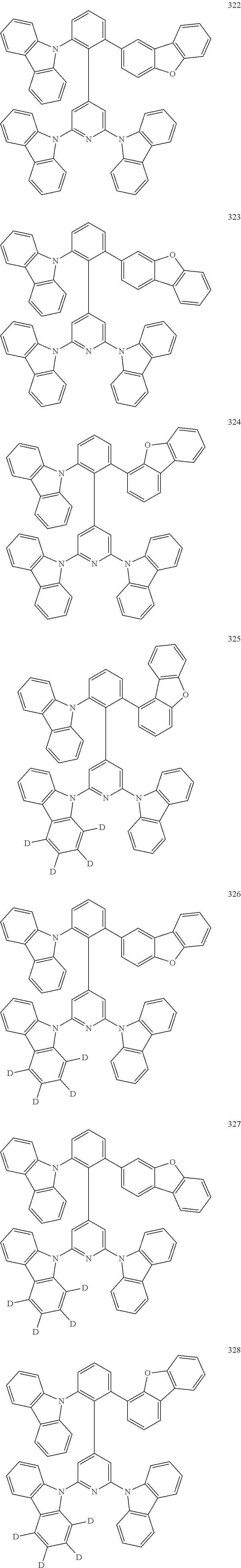

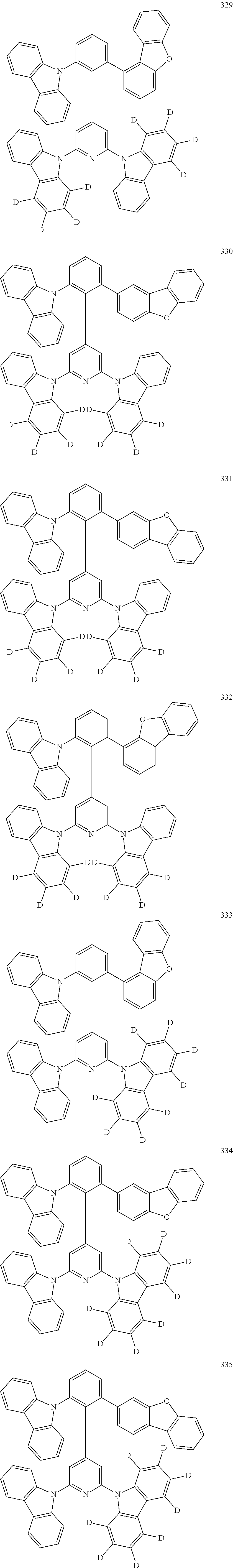

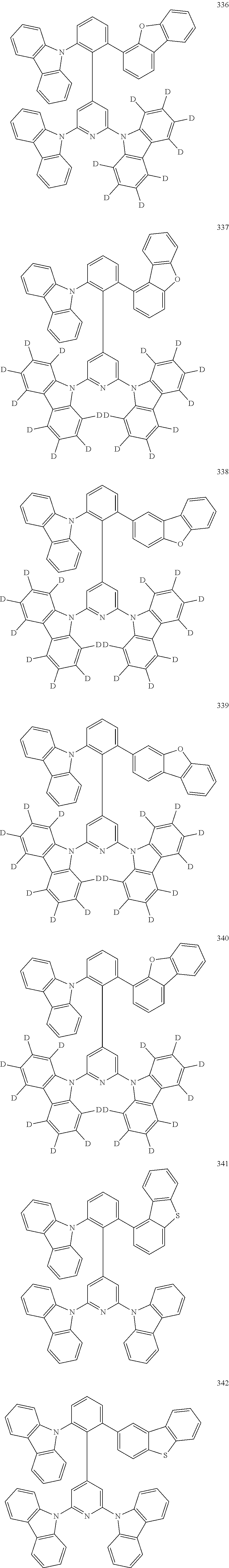

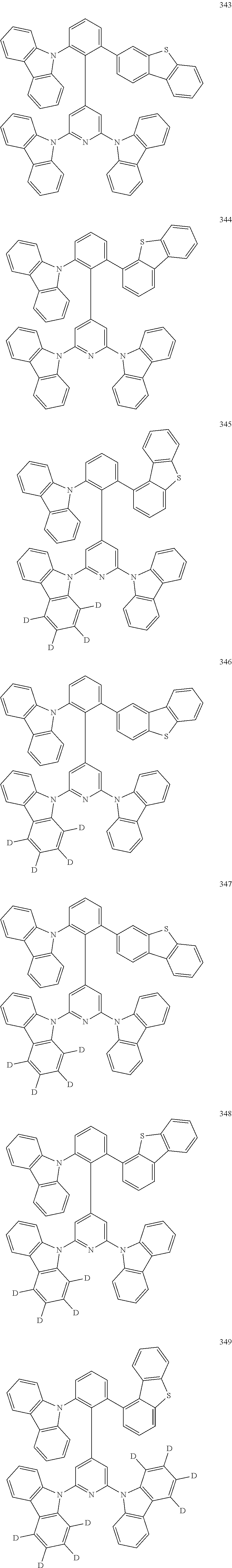

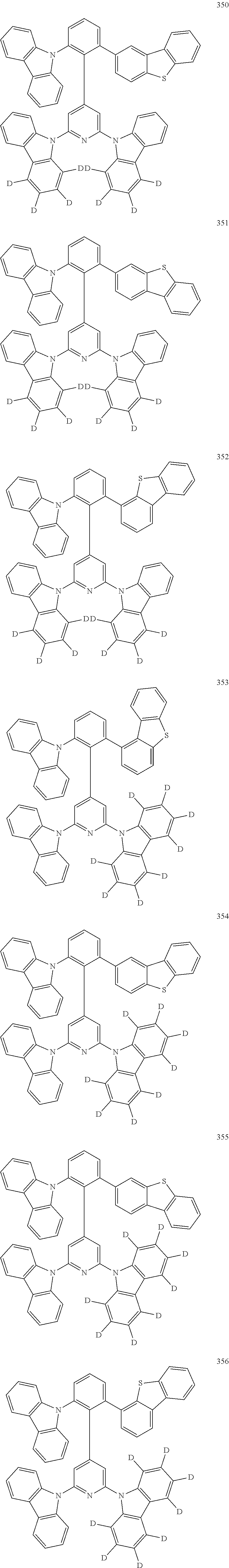

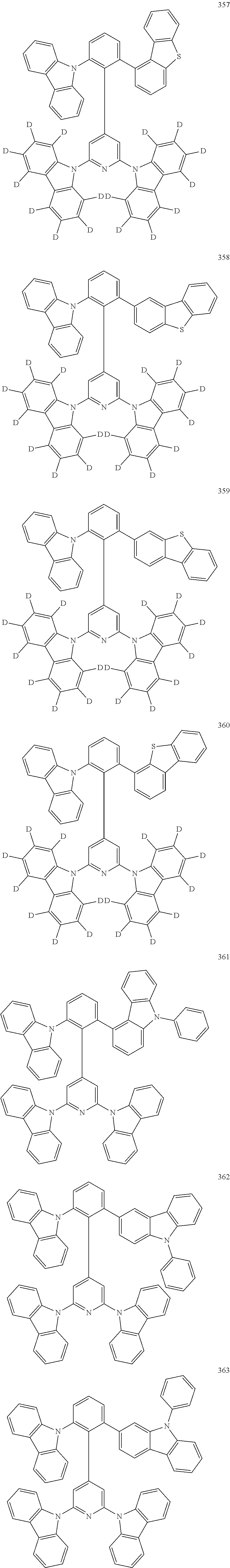

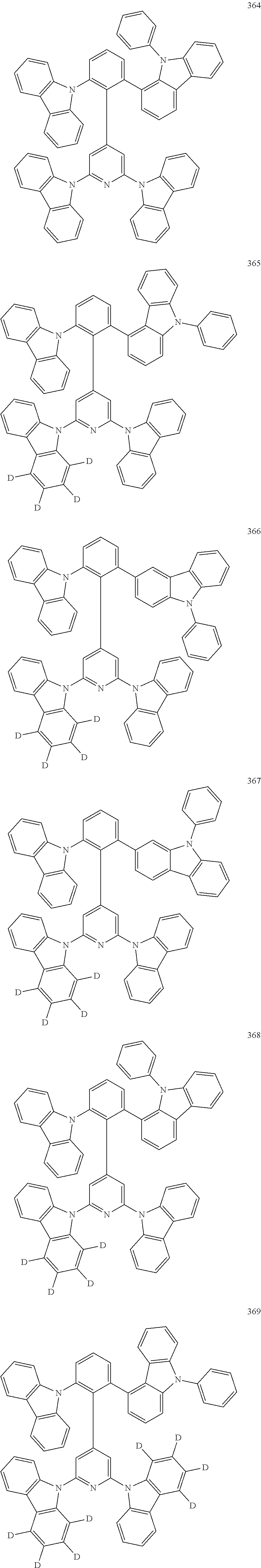

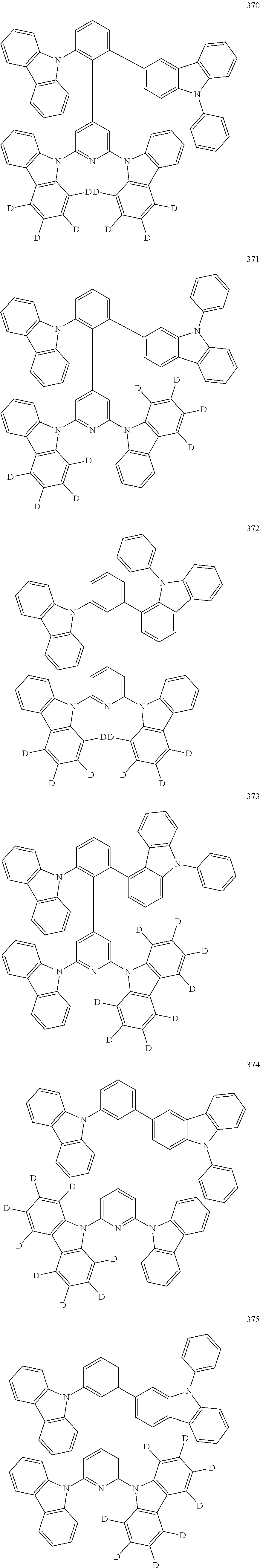

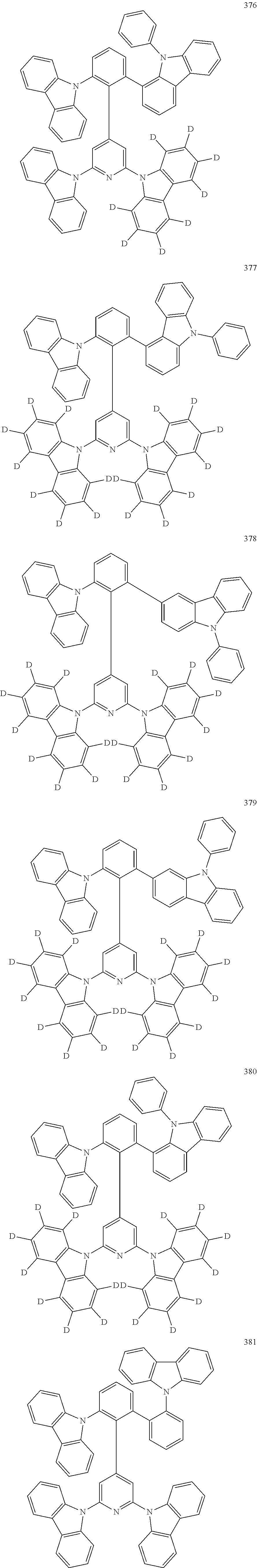

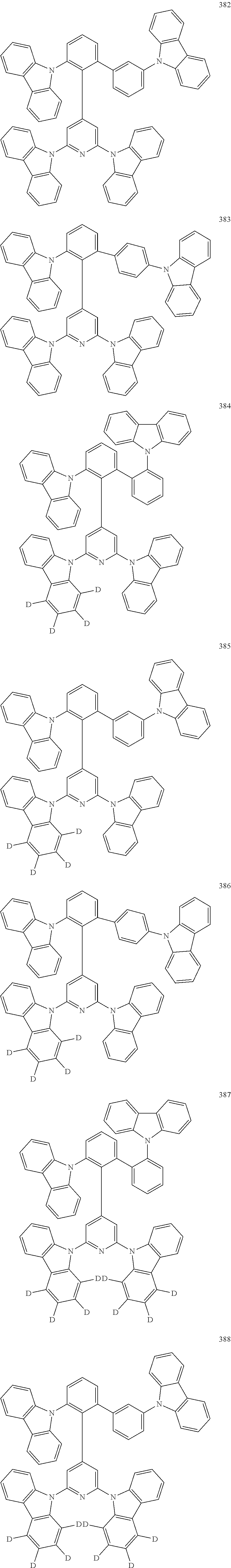

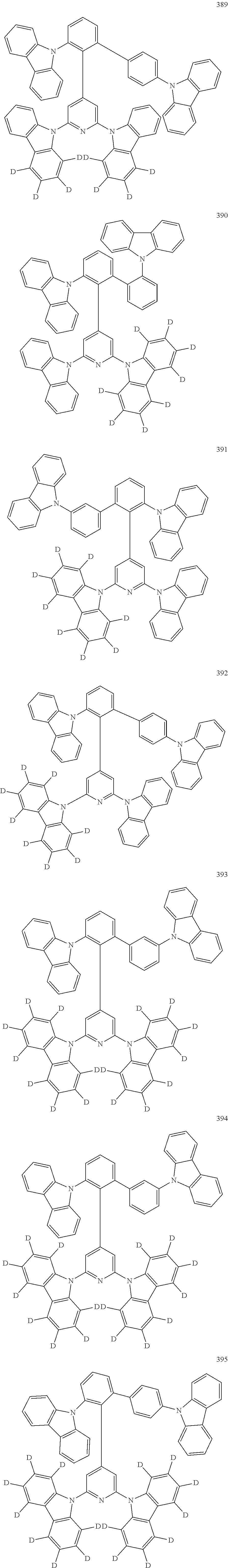

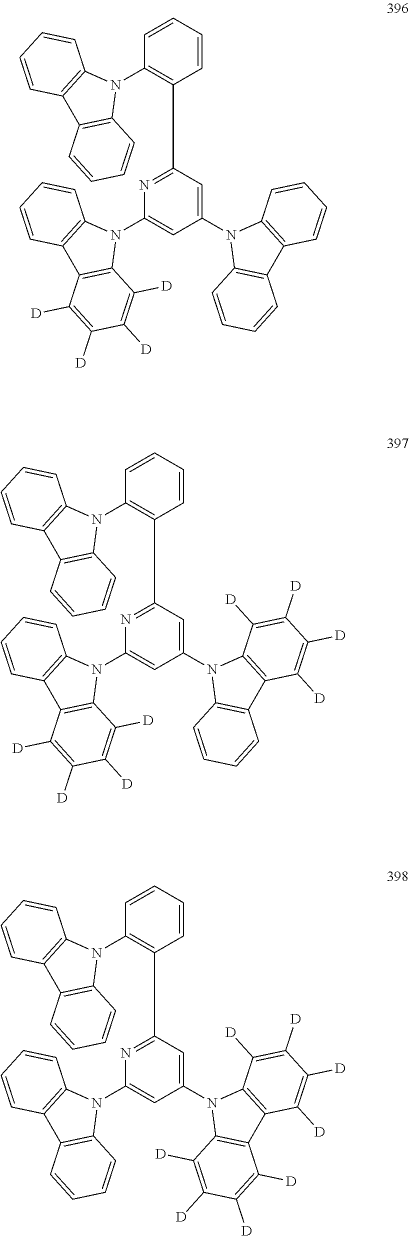 [SMILES: [2H]c1c([2H])c([2H])c2c(c1[2H])c1ccccc1n2-c1cc(-n2c3ccccc3c3ccccc32)cc(-c2ccccc2-n2c3ccccc3c3ccccc32)n1 ; [2H]c1c([2H])c([2H])c2c(c1[2H])c1ccccc1n2-c1cc(-c2ccccc2-n2c3ccccc3c3ccccc32)nc(-n2c3ccccc3c3c([2H])c([2H])c([2H])c([2H])c32)c1 ; [2H]c1c([2H])c([2H])c2c(c1[2H])c1c([2H])c([2H])c([2H])c([2H])c1n2-c1cc(-c2ccccc2-n2c3ccccc3c3ccccc32)nc(-n2c3ccccc3c3ccccc32)c1]

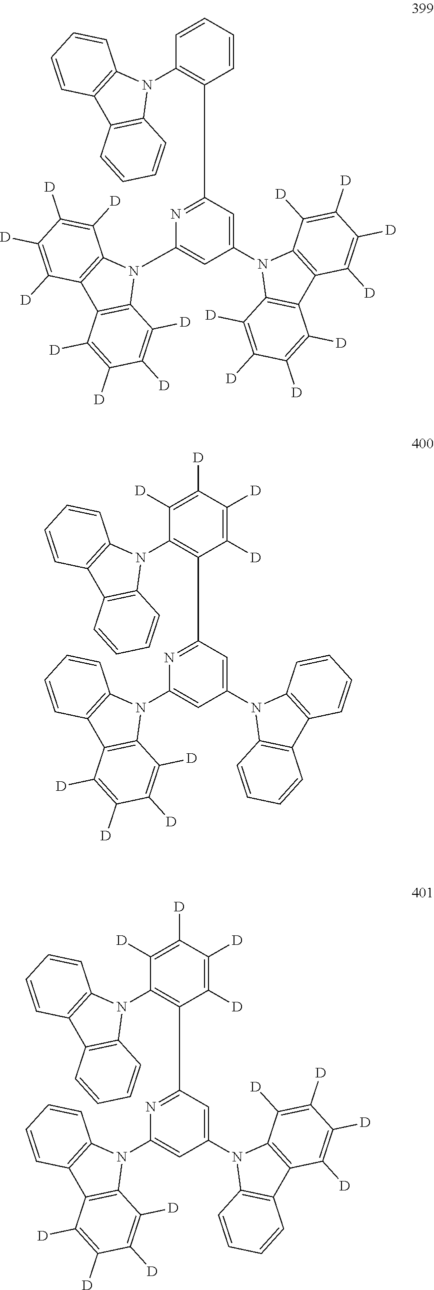 [SMILES: [2H]c1c([2H])c([2H])c2c(c1[2H])c1c([2H])c([2H])c([2H])c([2H])c1n2-c1cc(-c2ccccc2-n2c3ccccc3c3ccccc32)nc(-n2c3c([2H])c([2H])c([2H])c([2H])c3c3c([2H])c([2H])c([2H])c([2H])c32)c1 ; [2H]c1c([2H])c([2H])c(-n2c3ccccc3c3ccccc32)c(-c2cc(-n3c4ccccc4c4ccccc43)cc(-n3c4ccccc4c4c([2H])c([2H])c([2H])c([2H])c43)n2)c1[2H] ; [2H]c1c([2H])c([2H])c(-n2c3ccccc3c3ccccc32)c(-c2cc(-n3c4ccccc4c4c([2H])c([2H])c([2H])c([2H])c43)cc(-n3c4ccccc4c4c([2H])c([2H])c([2H])c([2H])c43)n2)c1[2H]]

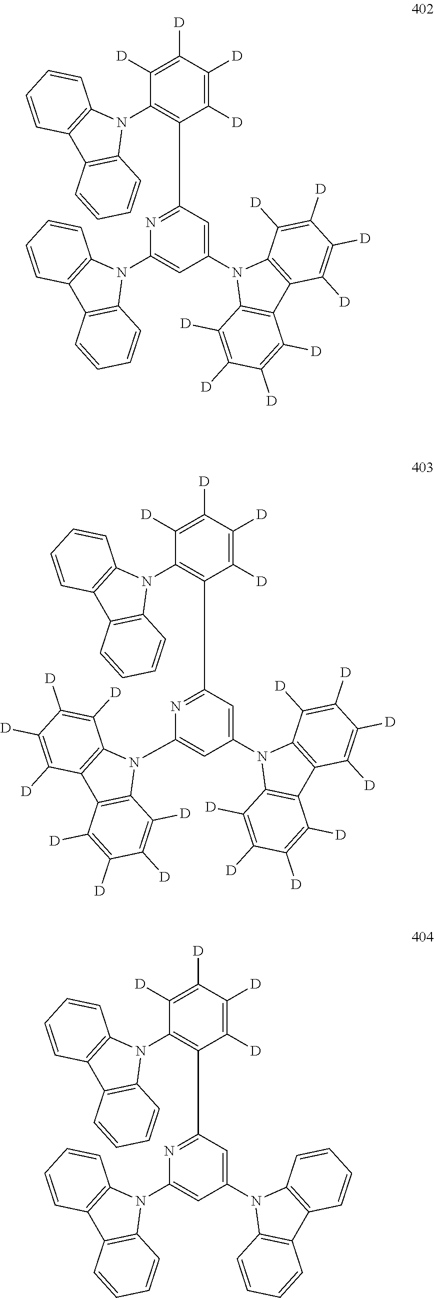 [SMILES: [2H]c1c([2H])c([2H])c(-n2c3ccccc3c3ccccc32)c(-c2cc(-n3c4c([2H])c([2H])c([2H])c([2H])c4c4c([2H])c([2H])c([2H])c([2H])c43)cc(-n3c4ccccc4c4ccccc43)n2)c1[2H] ; [2H]c1c([2H])c([2H])c(-n2c3ccccc3c3ccccc32)c(-c2cc(-n3c4c([2H])c([2H])c([2H])c([2H])c4c4c([2H])c([2H])c([2H])c([2H])c43)cc(-n3c4c([2H])c([2H])c([2H])c([2H])c4c4c([2H])c([2H])c([2H])c([2H])c43)n2)c1[2H] ; [2H]c1c([2H])c([2H])c(-n2c3ccccc3c3ccccc32)c(-c2cc(-n3c4ccccc4c4ccccc43)cc(-n3c4ccccc4c4ccccc43)n2)c1[2H]]

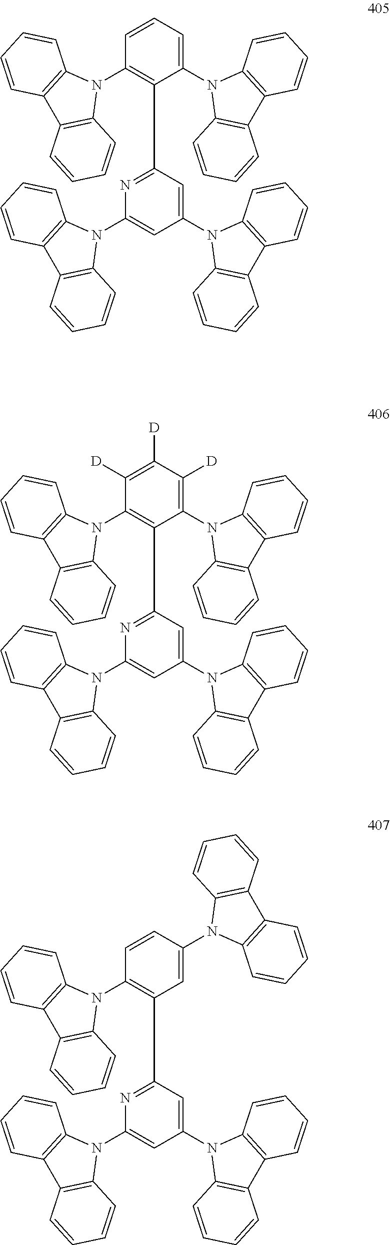 [SMILES: c1cc(-n2c3ccccc3c3ccccc32)c(-c2cc(-n3c4ccccc4c4ccccc43)cc(-n3c4ccccc4c4ccccc43)n2)c(-n2c3ccccc3c3ccccc32)c1 ; [2H]c1c([2H])c(-n2c3ccccc3c3ccccc32)c(-c2cc(-n3c4ccccc4c4ccccc43)cc(-n3c4ccccc4c4ccccc43)n2)c(-n2c3ccccc3c3ccccc32)c1[2H] ; c1ccc2c(c1)c1ccccc1n2-c1cc(-c2cc(-n3c4ccccc4c4ccccc43)ccc2-n2c3ccccc3c3ccccc32)nc(-n2c3ccccc3c3ccccc32)c1]

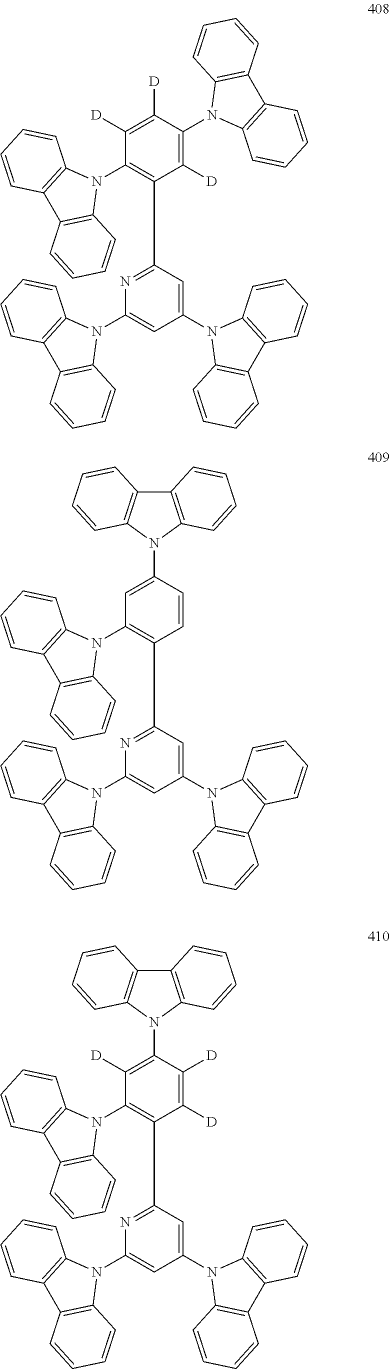 [SMILES: [2H]c1c([2H])c(-n2c3ccccc3c3ccccc32)c(-c2cc(-n3c4ccccc4c4ccccc43)cc(-n3c4ccccc4c4ccccc43)n2)c([2H])c1-n1c2ccccc2c2ccccc21 ; c1ccc2c(c1)c1ccccc1n2-c1cc(-c2ccc(-n3c4ccccc4c4ccccc43)cc2-n2c3ccccc3c3ccccc32)nc(-n2c3ccccc3c3ccccc32)c1 ; [2H]c1c([2H])c(-n2c3ccccc3c3ccccc32)c([2H])c(-n2c3ccccc3c3ccccc32)c1-c1cc(-n2c3ccccc3c3ccccc32)cc(-n2c3ccccc3c3ccccc32)n1]

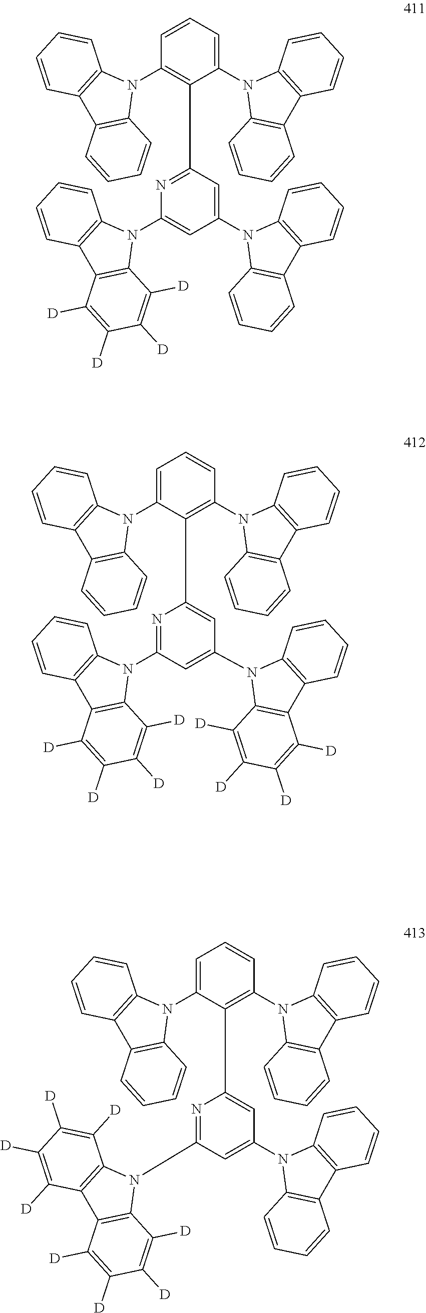 [SMILES: [2H]c1c([2H])c([2H])c2c(c1[2H])c1ccccc1n2-c1cc(-n2c3ccccc3c3ccccc32)cc(-c2c(-n3c4ccccc4c4ccccc43)cccc2-n2c3ccccc3c3ccccc32)n1 ; [2H]c1c([2H])c([2H])c2c(c1[2H])c1ccccc1n2-c1cc(-c2c(-n3c4ccccc4c4ccccc43)cccc2-n2c3ccccc3c3ccccc32)nc(-n2c3ccccc3c3c([2H])c([2H])c([2H])c([2H])c32)c1 ; [2H]c1c([2H])c([2H])c2c(c1[2H])c1c([2H])c([2H])c([2H])c([2H])c1n2-c1cc(-n2c3ccccc3c3ccccc32)cc(-c2c(-n3c4ccccc4c4ccccc43)cccc2-n2c3ccccc3c3ccccc32)n1]

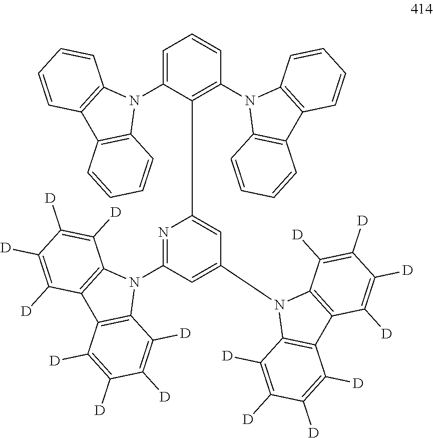 [SMILES: [2H]c1c([2H])c([2H])c2c(c1[2H])c1c([2H])c([2H])c([2H])c([2H])c1n2-c1cc(-c2c(-n3c4ccccc4c4ccccc43)cccc2-n2c3ccccc3c3ccccc32)nc(-n2c3c([2H])c([2H])c([2H])c([2H])c3c3c([2H])c([2H])c([2H])c([2H])c32)c1]

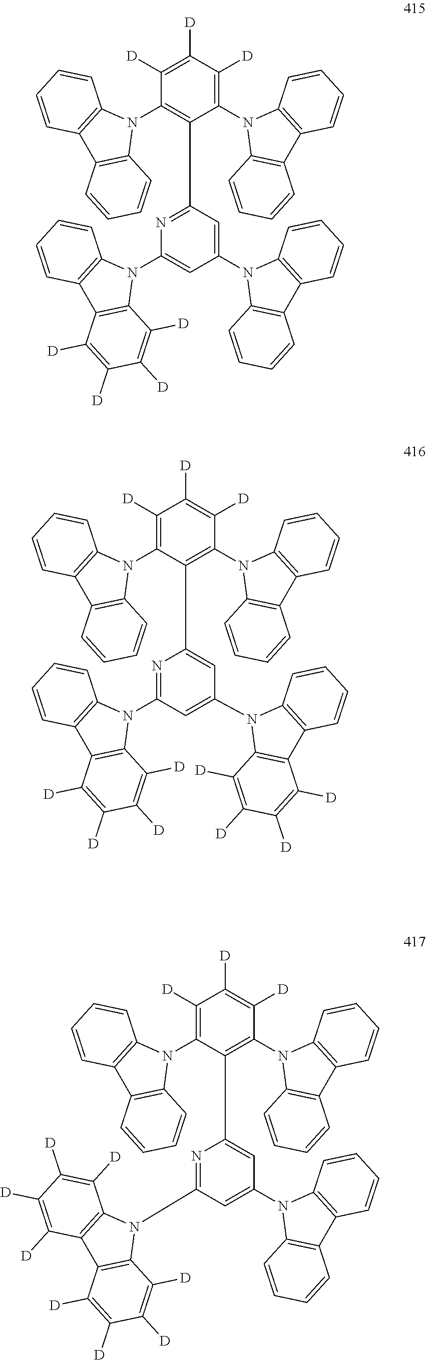 [SMILES: [2H]c1c([2H])c(-n2c3ccccc3c3ccccc32)c(-c2cc(-n3c4ccccc4c4ccccc43)cc(-n3c4ccccc4c4c([2H])c([2H])c([2H])c([2H])c43)n2)c(-n2c3ccccc3c3ccccc32)c1[2H] ; [2H]c1c([2H])c(-n2c3ccccc3c3ccccc32)c(-c2cc(-n3c4ccccc4c4c([2H])c([2H])c([2H])c([2H])c43)cc(-n3c4ccccc4c4c([2H])c([2H])c([2H])c([2H])c43)n2)c(-n2c3ccccc3c3ccccc32)c1[2H] ; [2H]c1c([2H])c(-n2c3ccccc3c3ccccc32)c(-c2cc(-n3c4ccccc4c4ccccc43)cc(-n3c4c([2H])c([2H])c([2H])c([2H])c4c4c([2H])c([2H])c([2H])c([2H])c43)n2)c(-n2c3ccccc3c3ccccc32)c1[2H]]

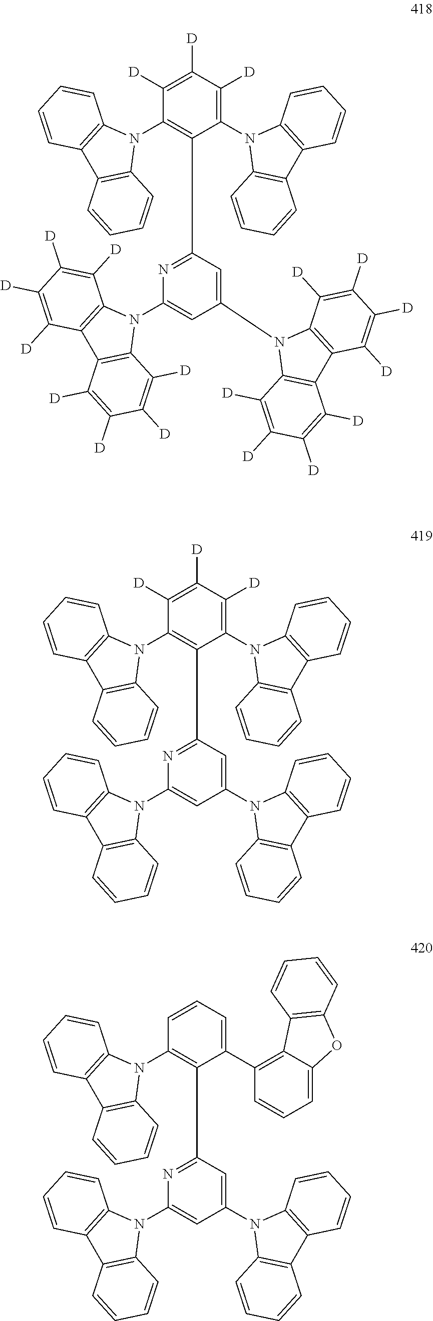 [SMILES: [2H]c1c([2H])c(-n2c3ccccc3c3ccccc32)c(-c2cc(-n3c4c([2H])c([2H])c([2H])c([2H])c4c4c([2H])c([2H])c([2H])c([2H])c43)cc(-n3c4c([2H])c([2H])c([2H])c([2H])c4c4c([2H])c([2H])c([2H])c([2H])c43)n2)c(-n2c3ccccc3c3ccccc32)c1[2H] ; [2H]c1c([2H])c(-n2c3ccccc3c3ccccc32)c(-c2cc(-n3c4ccccc4c4ccccc43)cc(-n3c4ccccc4c4ccccc43)n2)c(-n2c3ccccc3c3ccccc32)c1[2H] ; c1cc(-c2cccc3oc4ccccc4c23)c(-c2cc(-n3c4ccccc4c4ccccc43)cc(-n3c4ccccc4c4ccccc43)n2)c(-n2c3ccccc3c3ccccc32)c1]

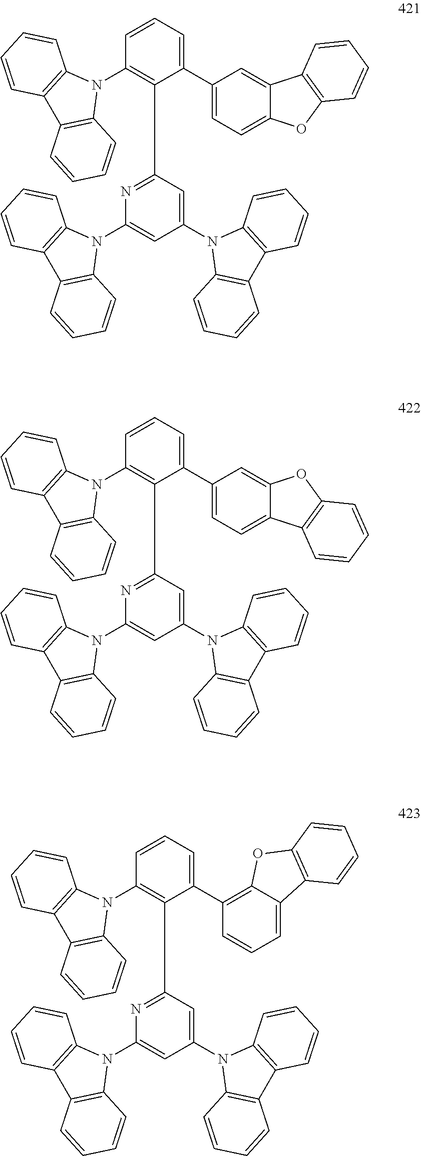 [SMILES: c1cc(-c2ccc3oc4ccccc4c3c2)c(-c2cc(-n3c4ccccc4c4ccccc43)cc(-n3c4ccccc4c4ccccc43)n2)c(-n2c3ccccc3c3ccccc32)c1 ; c1cc(-c2ccc3c(c2)oc2ccccc23)c(-c2cc(-n3c4ccccc4c4ccccc43)cc(-n3c4ccccc4c4ccccc43)n2)c(-n2c3ccccc3c3ccccc32)c1 ; c1cc(-c2cccc3c2oc2ccccc23)c(-c2cc(-n3c4ccccc4c4ccccc43)cc(-n3c4ccccc4c4ccccc43)n2)c(-n2c3ccccc3c3ccccc32)c1]

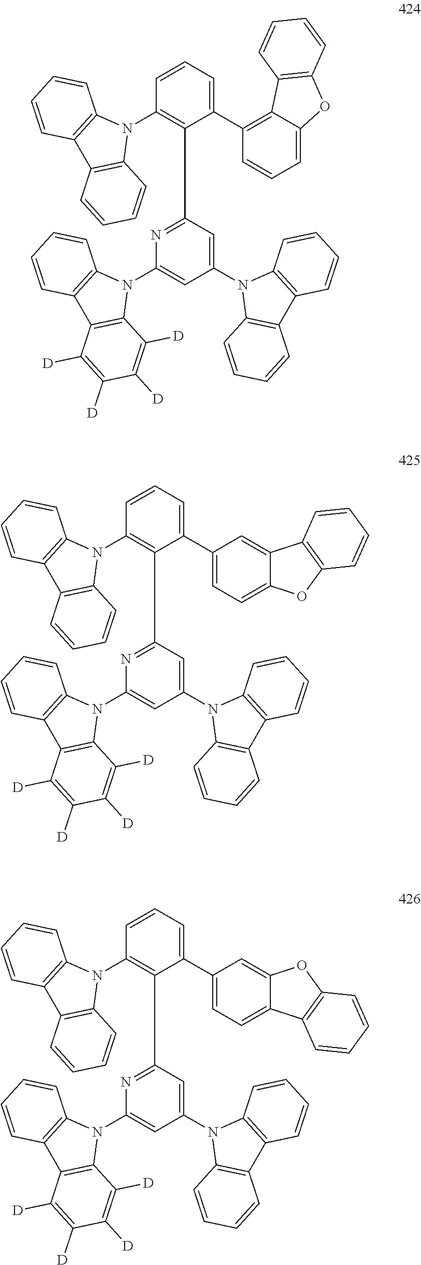 [SMILES: [2H]c1c([2H])c([2H])c2c(c1[2H])c1ccccc1n2-c1cc(-n2c3ccccc3c3ccccc32)cc(-c2c(-c3cccc4oc5ccccc5c34)cccc2-n2c3ccccc3c3ccccc32)n1 ; [2H]c1c([2H])c([2H])c2c(c1[2H])c1ccccc1n2-c1cc(-n2c3ccccc3c3ccccc32)cc(-c2c(-c3ccc4oc5ccccc5c4c3)cccc2-n2c3ccccc3c3ccccc32)n1 ; [2H]c1c([2H])c([2H])c2c(c1[2H])c1ccccc1n2-c1cc(-n2c3ccccc3c3ccccc32)cc(-c2c(-c3ccc4c(c3)oc3ccccc34)cccc2-n2c3ccccc3c3ccccc32)n1]

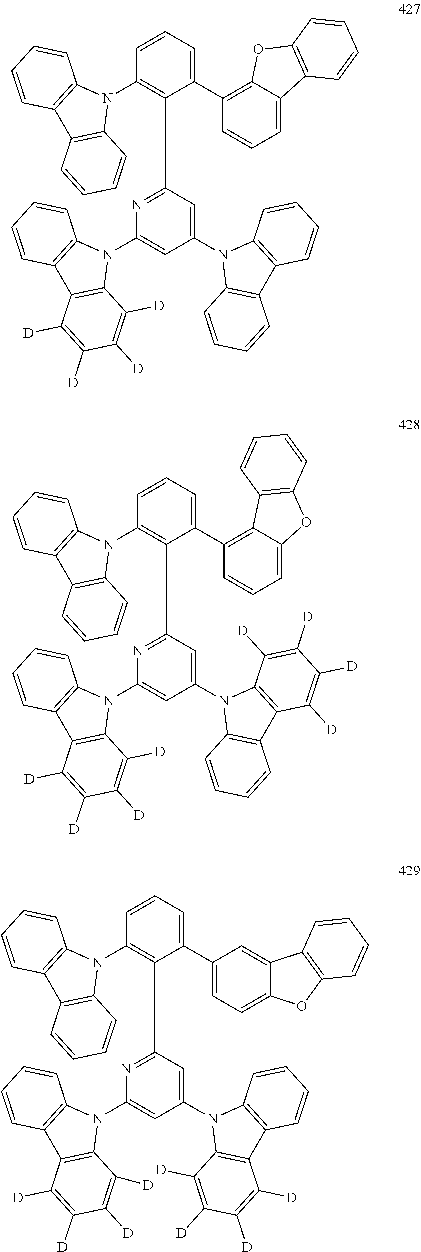 [SMILES: [2H]c1c([2H])c([2H])c2c(c1[2H])c1ccccc1n2-c1cc(-n2c3ccccc3c3ccccc32)cc(-c2c(-c3cccc4c3oc3ccccc34)cccc2-n2c3ccccc3c3ccccc32)n1 ; [2H]c1c([2H])c([2H])c2c(c1[2H])c1ccccc1n2-c1cc(-c2c(-c3cccc4oc5ccccc5c34)cccc2-n2c3ccccc3c3ccccc32)nc(-n2c3ccccc3c3c([2H])c([2H])c([2H])c([2H])c32)c1 ; [2H]c1c([2H])c([2H])c2c(c1[2H])c1ccccc1n2-c1cc(-c2c(-c3ccc4oc5ccccc5c4c3)cccc2-n2c3ccccc3c3ccccc32)nc(-n2c3ccccc3c3c([2H])c([2H])c([2H])c([2H])c32)c1]

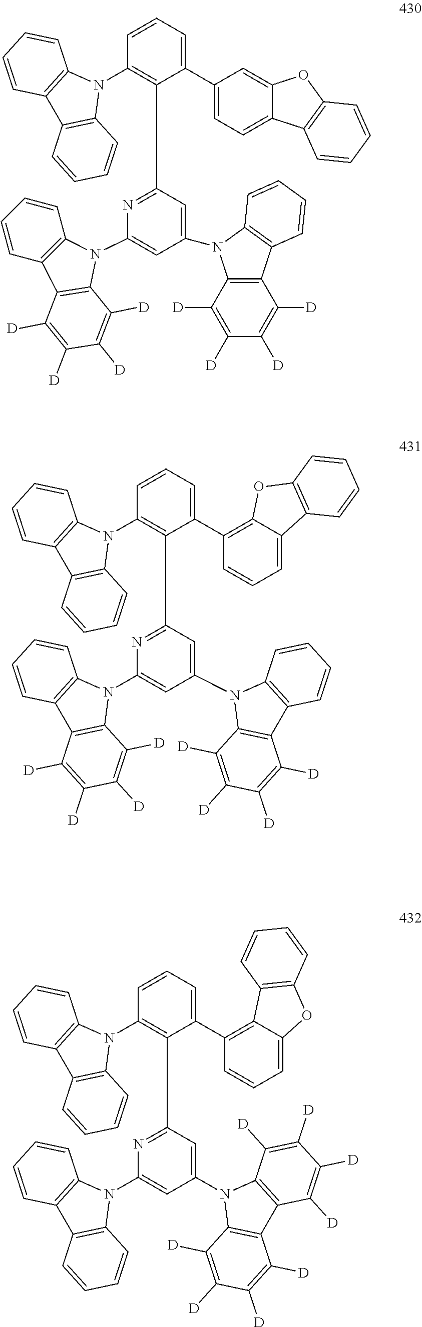 [SMILES: [2H]c1c([2H])c([2H])c2c(c1[2H])c1ccccc1n2-c1cc(-c2c(-c3ccc4c(c3)oc3ccccc34)cccc2-n2c3ccccc3c3ccccc32)nc(-n2c3ccccc3c3c([2H])c([2H])c([2H])c([2H])c32)c1 ; [2H]c1c([2H])c([2H])c2c(c1[2H])c1ccccc1n2-c1cc(-c2c(-c3cccc4c3oc3ccccc34)cccc2-n2c3ccccc3c3ccccc32)nc(-n2c3ccccc3c3c([2H])c([2H])c([2H])c([2H])c32)c1 ; [2H]c1c([2H])c([2H])c2c(c1[2H])c1c([2H])c([2H])c([2H])c([2H])c1n2-c1cc(-c2c(-c3cccc4oc5ccccc5c34)cccc2-n2c3ccccc3c3ccccc32)nc(-n2c3ccccc3c3ccccc32)c1]

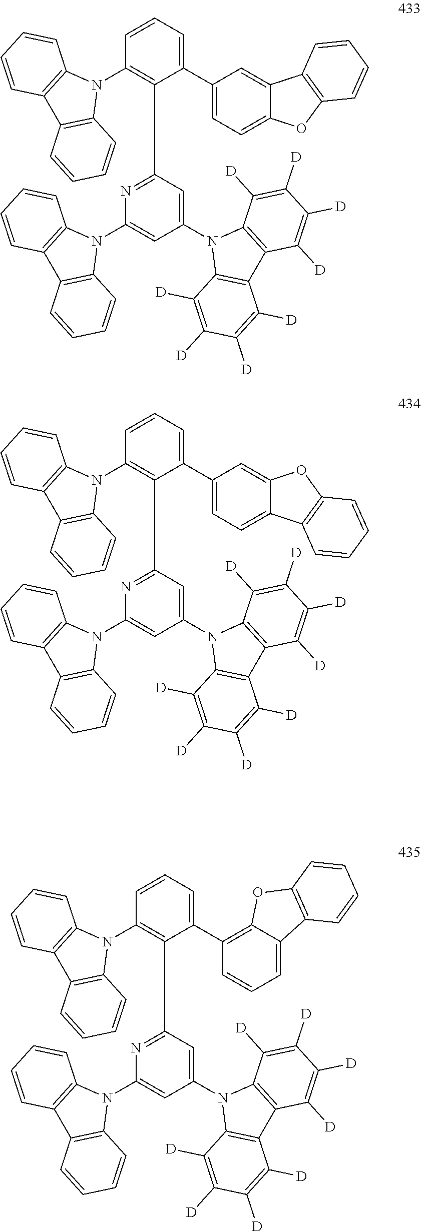 [SMILES: [2H]c1c([2H])c([2H])c2c(c1[2H])c1c([2H])c([2H])c([2H])c([2H])c1n2-c1cc(-c2c(-c3ccc4oc5ccccc5c4c3)cccc2-n2c3ccccc3c3ccccc32)nc(-n2c3ccccc3c3ccccc32)c1 ; [2H]c1c([2H])c([2H])c2c(c1[2H])c1c([2H])c([2H])c([2H])c([2H])c1n2-c1cc(-c2c(-c3ccc4c(c3)oc3ccccc34)cccc2-n2c3ccccc3c3ccccc32)nc(-n2c3ccccc3c3ccccc32)c1 ; [2H]c1c([2H])c([2H])c2c(c1[2H])c1c([2H])c([2H])c([2H])c([2H])c1n2-c1cc(-c2c(-c3cccc4c3oc3ccccc34)cccc2-n2c3ccccc3c3ccccc32)nc(-n2c3ccccc3c3ccccc32)c1]

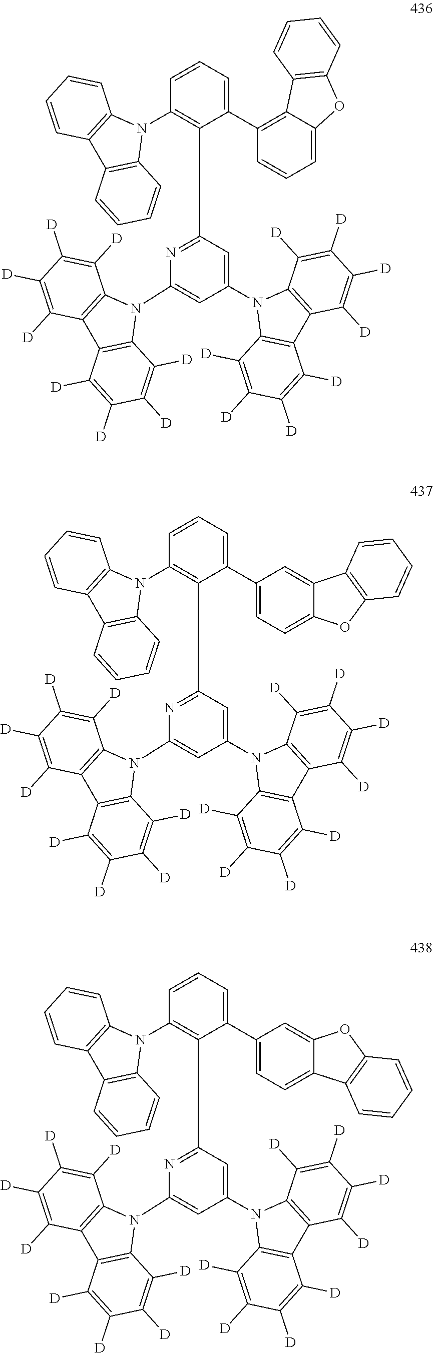

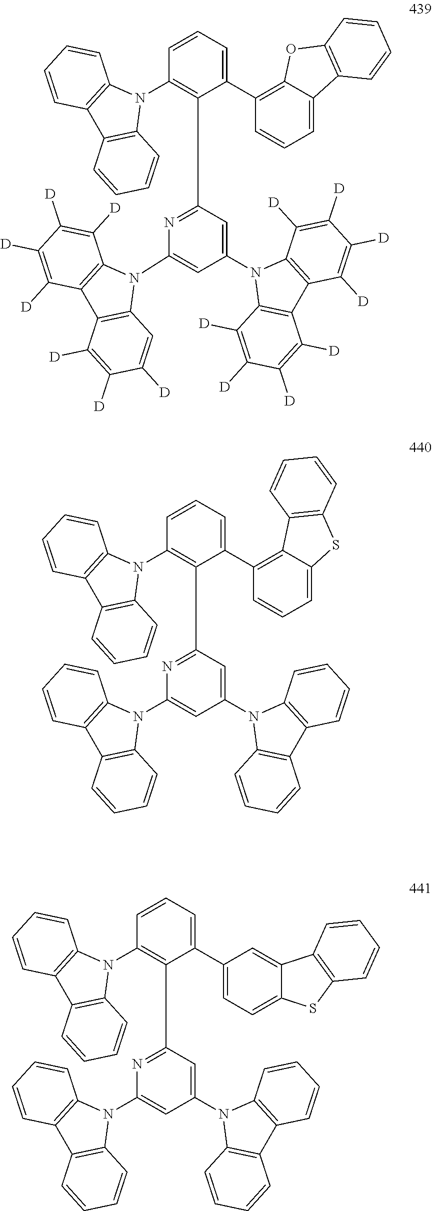 [SMILES: [2H]c1cc2c(c([2H])c1[2H])c1c([2H])c([2H])c([2H])c([2H])c1n2-c1cc(-n2c3c([2H])c([2H])c([2H])c([2H])c3c3c([2H])c([2H])c([2H])c([2H])c32)cc(-c2c(-c3cccc4c3oc3ccccc34)cccc2-n2c3ccccc3c3ccccc32)n1 ; c1cc(-c2cccc3sc4ccccc4c23)c(-c2cc(-n3c4ccccc4c4ccccc43)cc(-n3c4ccccc4c4ccccc43)n2)c(-n2c3ccccc3c3ccccc32)c1 ; c1cc(-c2ccc3sc4ccccc4c3c2)c(-c2cc(-n3c4ccccc4c4ccccc43)cc(-n3c4ccccc4c4ccccc43)n2)c(-n2c3ccccc3c3ccccc32)c1]

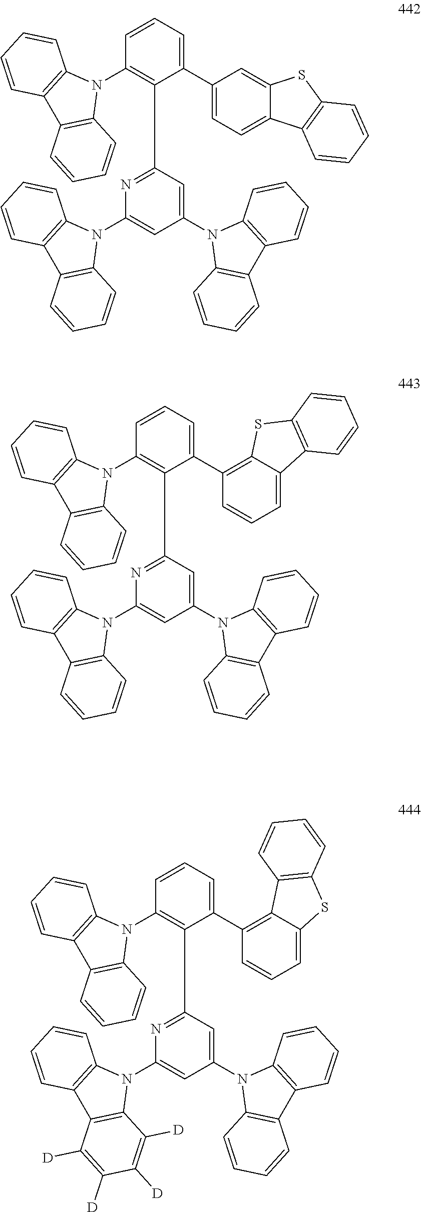 [SMILES: c1cc(-c2ccc3c(c2)sc2ccccc23)c(-c2cc(-n3c4ccccc4c4ccccc43)cc(-n3c4ccccc4c4ccccc43)n2)c(-n2c3ccccc3c3ccccc32)c1 ; c1cc(-c2cccc3c2sc2ccccc23)c(-c2cc(-n3c4ccccc4c4ccccc43)cc(-n3c4ccccc4c4ccccc43)n2)c(-n2c3ccccc3c3ccccc32)c1 ; [2H]c1c([2H])c([2H])c2c(c1[2H])c1ccccc1n2-c1cc(-n2c3ccccc3c3ccccc32)cc(-c2c(-c3cccc4sc5ccccc5c34)cccc2-n2c3ccccc3c3ccccc32)n1]

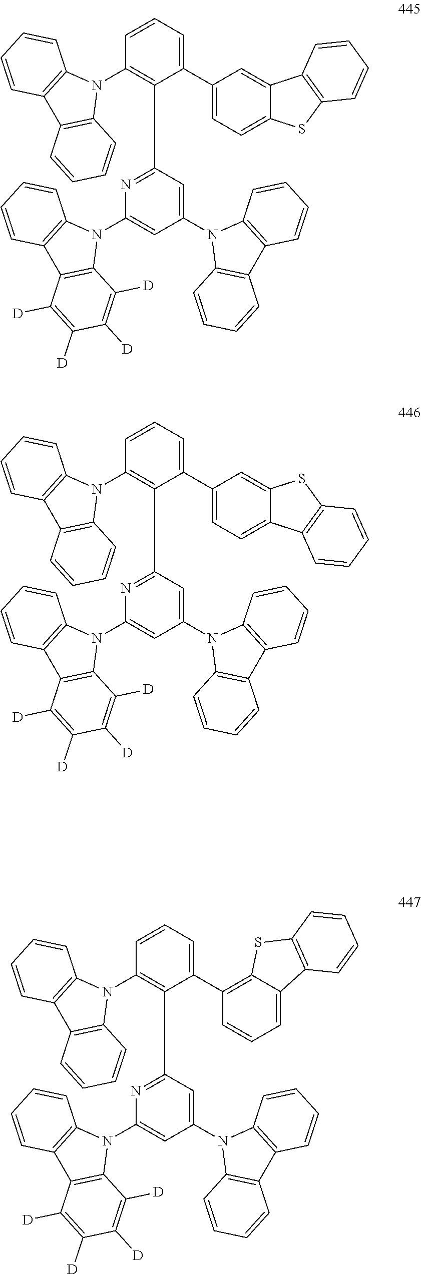 [SMILES: [2H]c1c([2H])c([2H])c2c(c1[2H])c1ccccc1n2-c1cc(-n2c3ccccc3c3ccccc32)cc(-c2c(-c3ccc4sc5ccccc5c4c3)cccc2-n2c3ccccc3c3ccccc32)n1 ; [2H]c1c([2H])c([2H])c2c(c1[2H])c1ccccc1n2-c1cc(-n2c3ccccc3c3ccccc32)cc(-c2c(-c3ccc4c(c3)sc3ccccc34)cccc2-n2c3ccccc3c3ccccc32)n1 ; [2H]c1c([2H])c([2H])c2c(c1[2H])c1ccccc1n2-c1cc(-n2c3ccccc3c3ccccc32)cc(-c2c(-c3cccc4c3sc3ccccc34)cccc2-n2c3ccccc3c3ccccc32)n1]

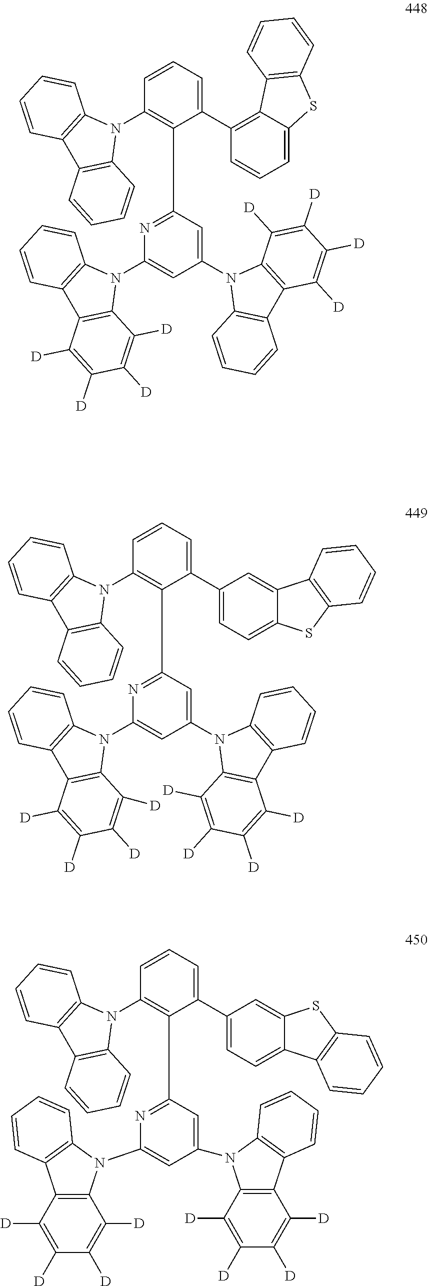 [SMILES: [2H]c1c([2H])c([2H])c2c(c1[2H])c1ccccc1n2-c1cc(-c2c(-c3cccc4sc5ccccc5c34)cccc2-n2c3ccccc3c3ccccc32)nc(-n2c3ccccc3c3c([2H])c([2H])c([2H])c([2H])c32)c1 ; [2H]c1c([2H])c([2H])c2c(c1[2H])c1ccccc1n2-c1cc(-c2c(-c3ccc4sc5ccccc5c4c3)cccc2-n2c3ccccc3c3ccccc32)nc(-n2c3ccccc3c3c([2H])c([2H])c([2H])c([2H])c32)c1 ; [2H]c1c([2H])c([2H])c2c(c1[2H])c1ccccc1n2-c1cc(-c2c(-c3ccc4c(c3)sc3ccccc34)cccc2-n2c3ccccc3c3ccccc32)nc(-n2c3ccccc3c3c([2H])c([2H])c([2H])c([2H])c32)c1]

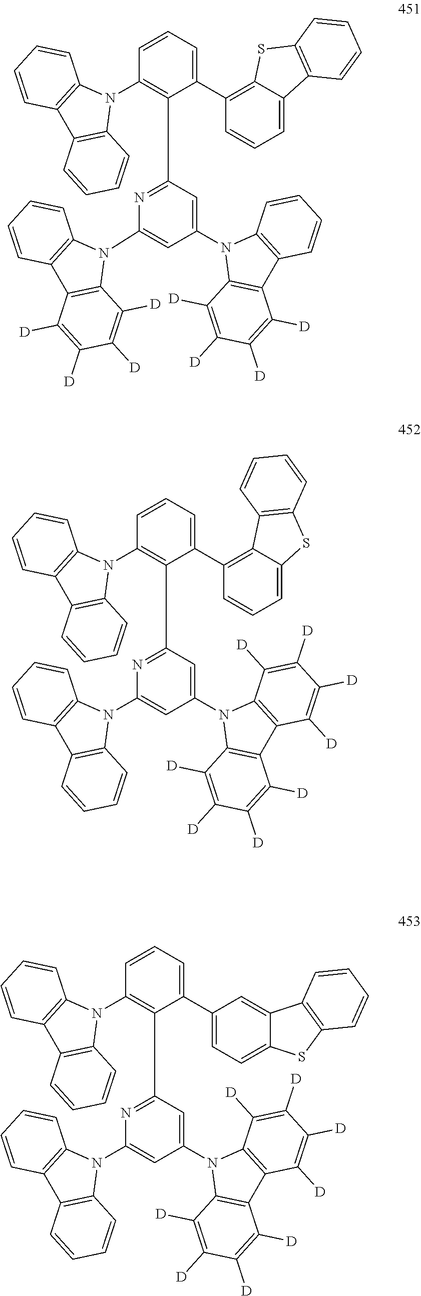 [SMILES: [2H]c1c([2H])c([2H])c2c(c1[2H])c1ccccc1n2-c1cc(-c2c(-c3cccc4c3sc3ccccc34)cccc2-n2c3ccccc3c3ccccc32)nc(-n2c3ccccc3c3c([2H])c([2H])c([2H])c([2H])c32)c1 ; [2H]c1c([2H])c([2H])c2c(c1[2H])c1c([2H])c([2H])c([2H])c([2H])c1n2-c1cc(-c2c(-c3cccc4sc5ccccc5c34)cccc2-n2c3ccccc3c3ccccc32)nc(-n2c3ccccc3c3ccccc32)c1 ; [2H]c1c([2H])c([2H])c2c(c1[2H])c1c([2H])c([2H])c([2H])c([2H])c1n2-c1cc(-c2c(-c3ccc4sc5ccccc5c4c3)cccc2-n2c3ccccc3c3ccccc32)nc(-n2c3ccccc3c3ccccc32)c1]

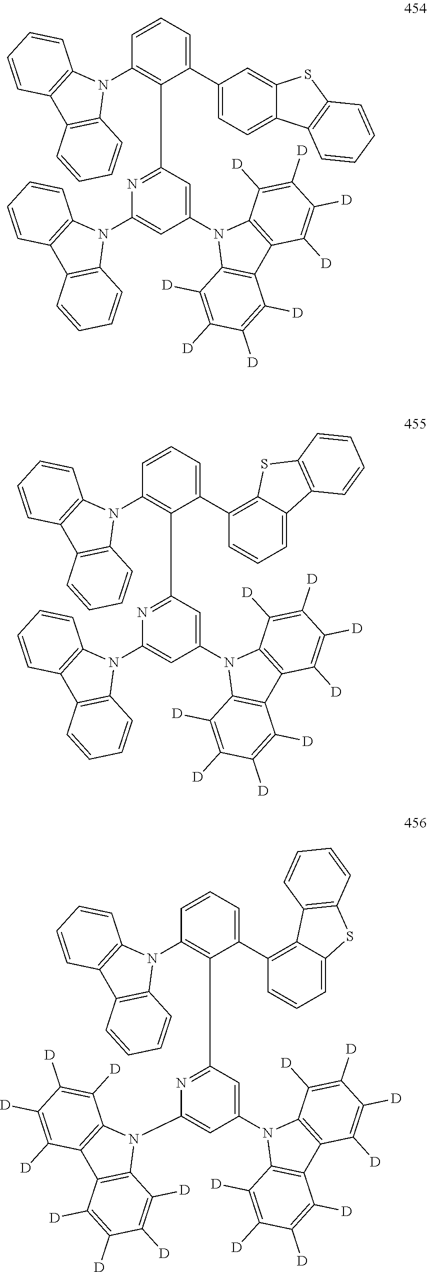

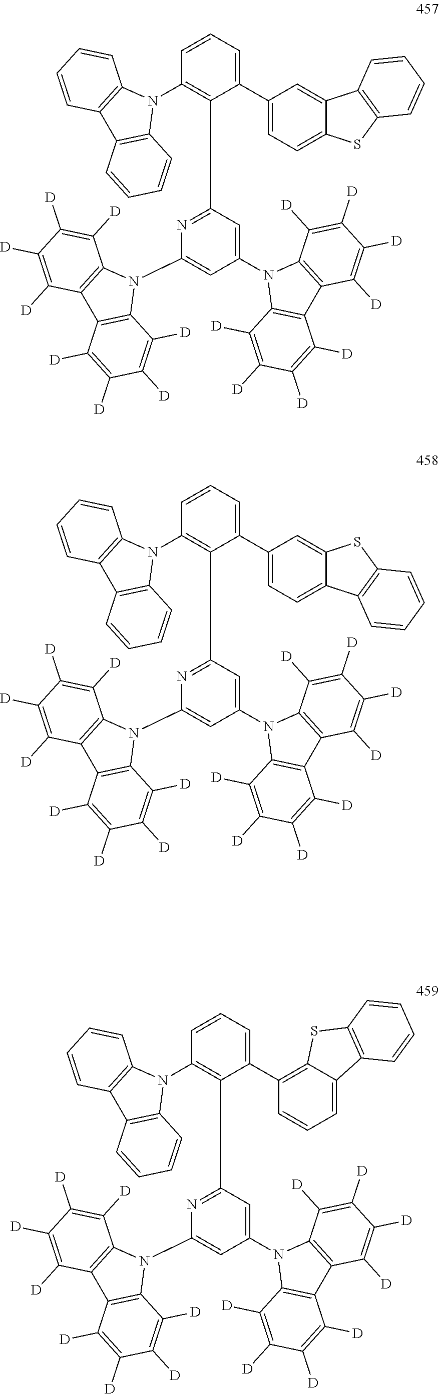

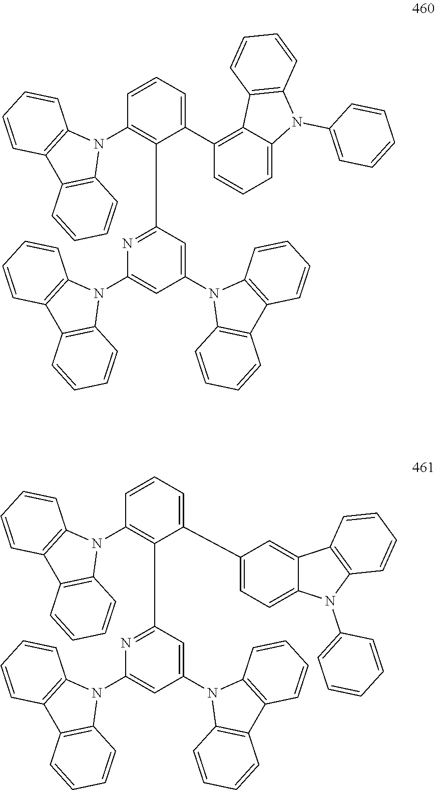 [SMILES: c1ccc(-n2c3ccccc3c3c(-c4cccc(-n5c6ccccc6c6ccccc65)c4-c4cc(-n5c6ccccc6c6ccccc65)cc(-n5c6ccccc6c6ccccc65)n4)cccc32)cc1 ; c1ccc(-n2c3ccccc3c3cc(-c4cccc(-n5c6ccccc6c6ccccc65)c4-c4cc(-n5c6ccccc6c6ccccc65)cc(-n5c6ccccc6c6ccccc65)n4)ccc32)cc1]

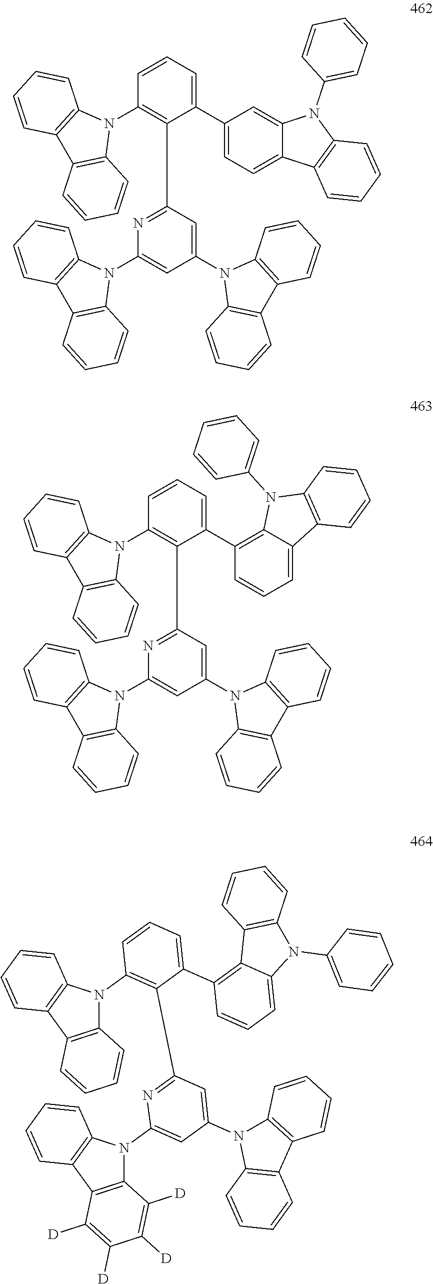 [SMILES: c1ccc(-n2c3ccccc3c3ccc(-c4cccc(-n5c6ccccc6c6ccccc65)c4-c4cc(-n5c6ccccc6c6ccccc65)cc(-n5c6ccccc6c6ccccc65)n4)cc32)cc1 ; c1ccc(-n2c3ccccc3c3cccc(-c4cccc(-n5c6ccccc6c6ccccc65)c4-c4cc(-n5c6ccccc6c6ccccc65)cc(-n5c6ccccc6c6ccccc65)n4)c32)cc1 ; [2H]c1c([2H])c([2H])c2c(c1[2H])c1ccccc1n2-c1cc(-n2c3ccccc3c3ccccc32)cc(-c2c(-c3cccc4c3c3ccccc3n4-c3ccccc3)cccc2-n2c3ccccc3c3ccccc32)n1]

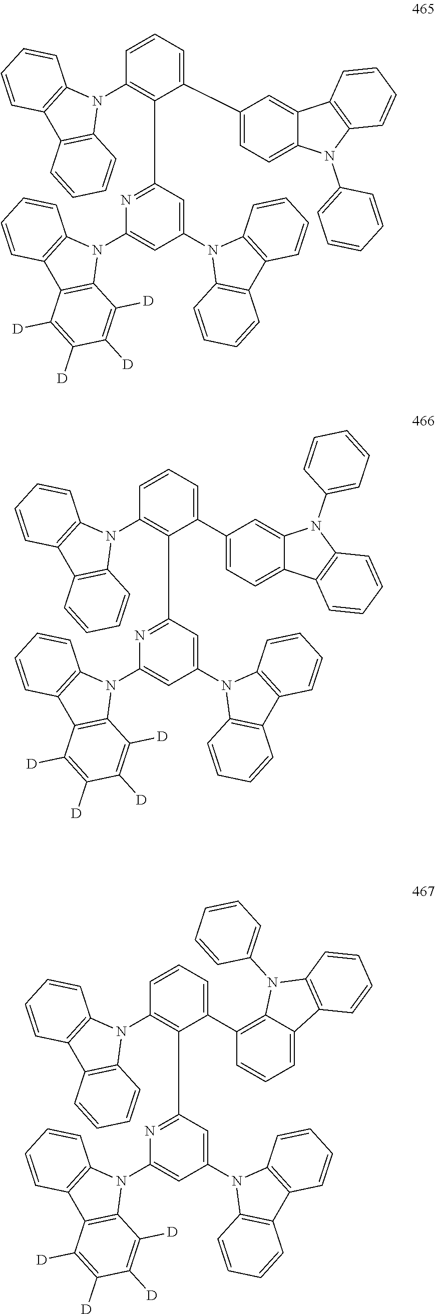 [SMILES: [2H]c1c([2H])c([2H])c2c(c1[2H])c1ccccc1n2-c1cc(-n2c3ccccc3c3ccccc32)cc(-c2c(-c3ccc4c(c3)c3ccccc3n4-c3ccccc3)cccc2-n2c3ccccc3c3ccccc32)n1 ; [2H]c1c([2H])c([2H])c2c(c1[2H])c1ccccc1n2-c1cc(-n2c3ccccc3c3ccccc32)cc(-c2c(-c3ccc4c5ccccc5n(-c5ccccc5)c4c3)cccc2-n2c3ccccc3c3ccccc32)n1 ; [2H]c1c([2H])c([2H])c2c(c1[2H])c1ccccc1n2-c1cc(-n2c3ccccc3c3ccccc32)cc(-c2c(-c3cccc4c5ccccc5n(-c5ccccc5)c34)cccc2-n2c3ccccc3c3ccccc32)n1]

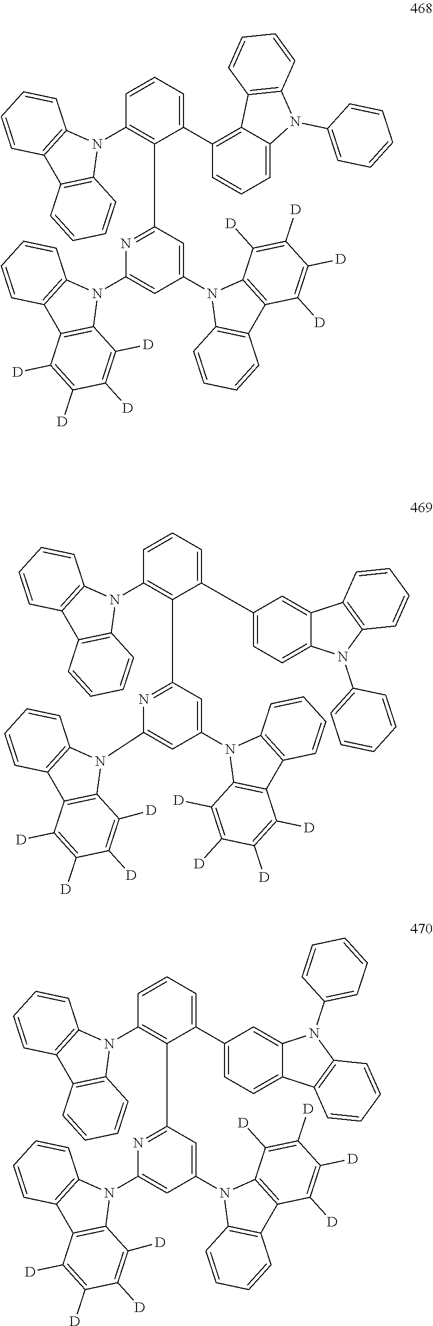 [SMILES: [2H]c1c([2H])c([2H])c2c(c1[2H])c1ccccc1n2-c1cc(-c2c(-c3cccc4c3c3ccccc3n4-c3ccccc3)cccc2-n2c3ccccc3c3ccccc32)nc(-n2c3ccccc3c3c([2H])c([2H])c([2H])c([2H])c32)c1 ; [2H]c1c([2H])c([2H])c2c(c1[2H])c1ccccc1n2-c1cc(-c2c(-c3ccc4c(c3)c3ccccc3n4-c3ccccc3)cccc2-n2c3ccccc3c3ccccc32)nc(-n2c3ccccc3c3c([2H])c([2H])c([2H])c([2H])c32)c1 ; [2H]c1c([2H])c([2H])c2c(c1[2H])c1ccccc1n2-c1cc(-c2c(-c3ccc4c5ccccc5n(-c5ccccc5)c4c3)cccc2-n2c3ccccc3c3ccccc32)nc(-n2c3ccccc3c3c([2H])c([2H])c([2H])c([2H])c32)c1]

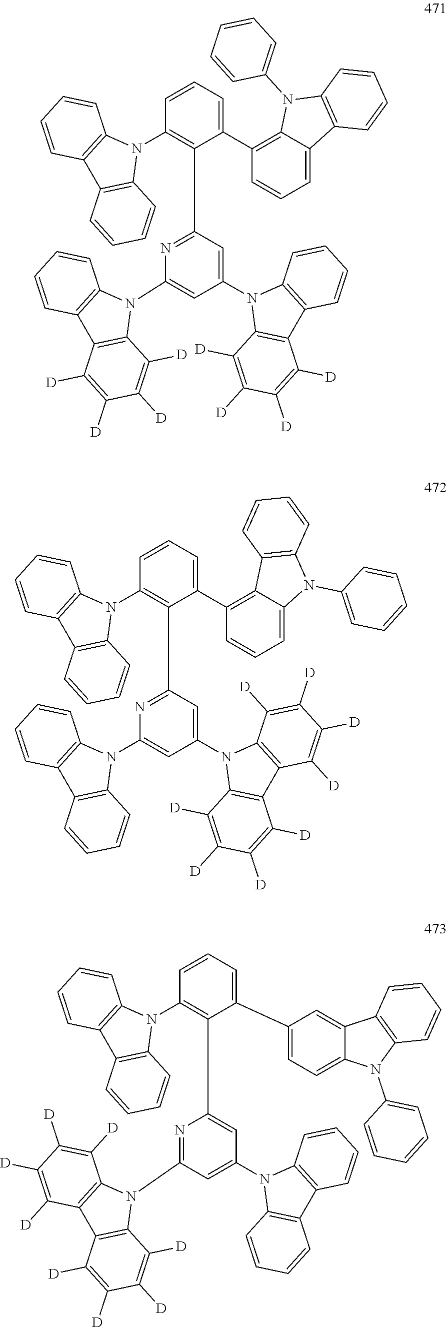 [SMILES: [2H]c1c([2H])c([2H])c2c(c1[2H])c1ccccc1n2-c1cc(-c2c(-c3cccc4c5ccccc5n(-c5ccccc5)c34)cccc2-n2c3ccccc3c3ccccc32)nc(-n2c3ccccc3c3c([2H])c([2H])c([2H])c([2H])c32)c1 ; [2H]c1c([2H])c([2H])c2c(c1[2H])c1c([2H])c([2H])c([2H])c([2H])c1n2-c1cc(-c2c(-c3cccc4c3c3ccccc3n4-c3ccccc3)cccc2-n2c3ccccc3c3ccccc32)nc(-n2c3ccccc3c3ccccc32)c1 ; [2H]c1c([2H])c([2H])c2c(c1[2H])c1c([2H])c([2H])c([2H])c([2H])c1n2-c1cc(-n2c3ccccc3c3ccccc32)cc(-c2c(-c3ccc4c(c3)c3ccccc3n4-c3ccccc3)cccc2-n2c3ccccc3c3ccccc32)n1]

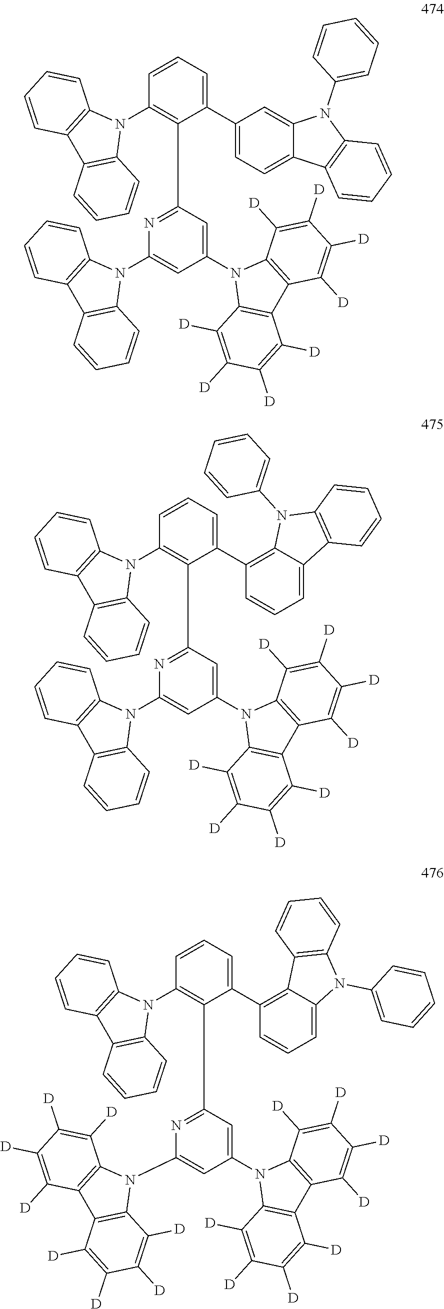

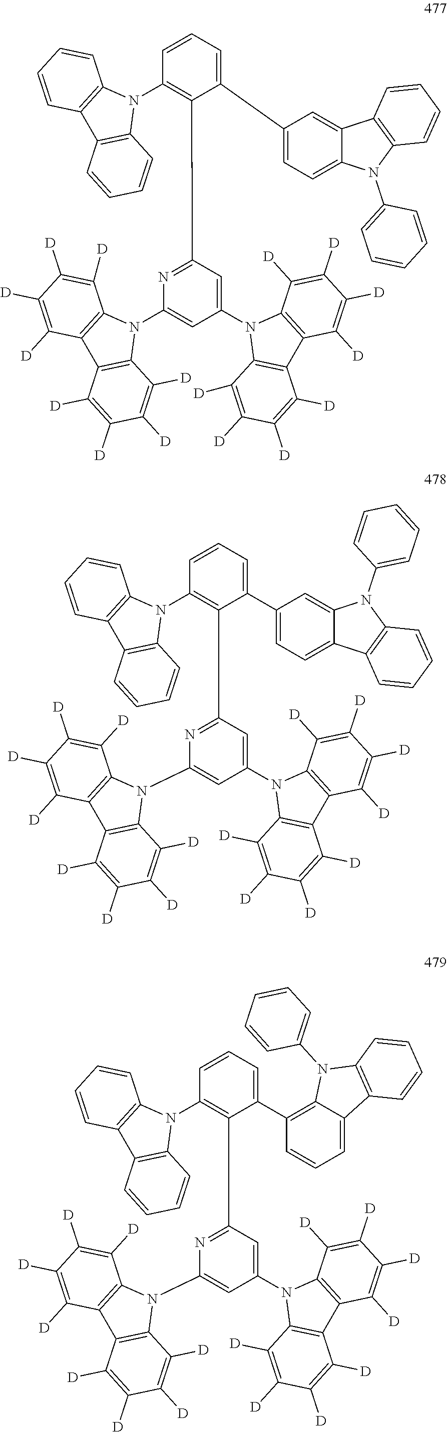

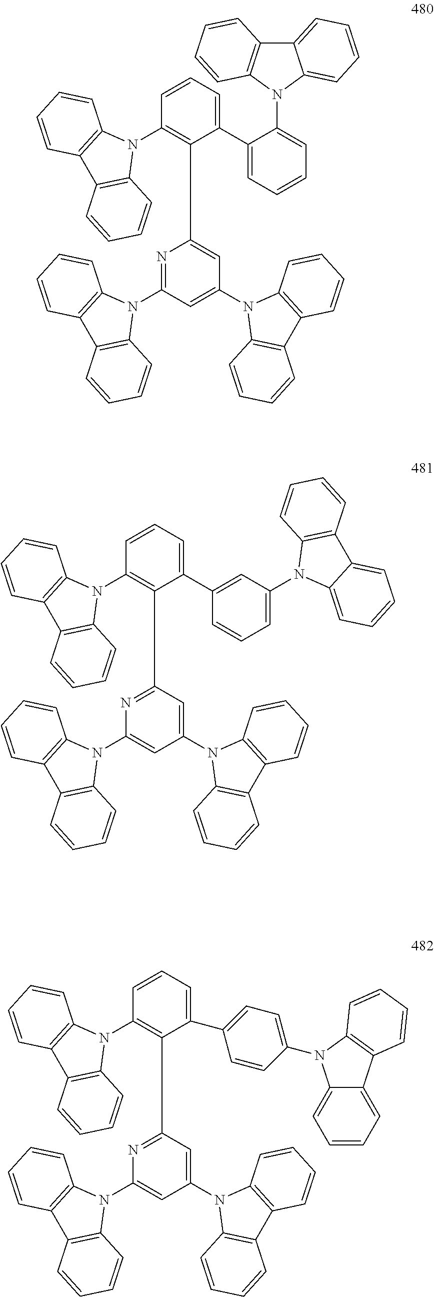 [SMILES: c1ccc(-n2c3ccccc3c3ccccc32)c(-c2cccc(-n3c4ccccc4c4ccccc43)c2-c2cc(-n3c4ccccc4c4ccccc43)cc(-n3c4ccccc4c4ccccc43)n2)c1 ; c1cc(-c2cccc(-n3c4ccccc4c4ccccc43)c2-c2cc(-n3c4ccccc4c4ccccc43)cc(-n3c4ccccc4c4ccccc43)n2)cc(-n2c3ccccc3c3ccccc32)c1 ; c1cc(-c2ccc(-n3c4ccccc4c4ccccc43)cc2)c(-c2cc(-n3c4ccccc4c4ccccc43)cc(-n3c4ccccc4c4ccccc43)n2)c(-n2c3ccccc3c3ccccc32)c1]

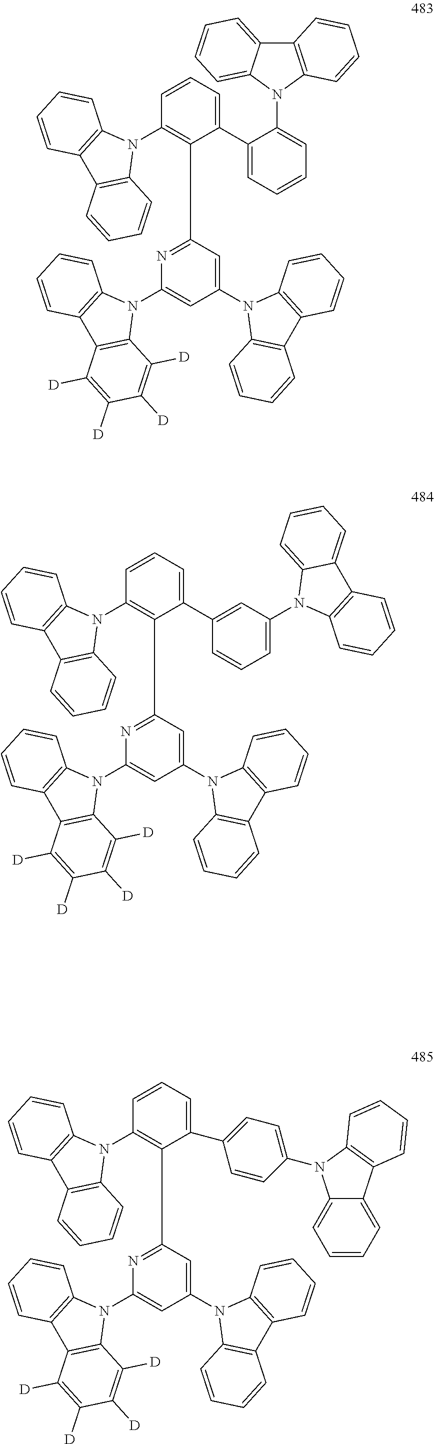 [SMILES: [2H]c1c([2H])c([2H])c2c(c1[2H])c1ccccc1n2-c1cc(-n2c3ccccc3c3ccccc32)cc(-c2c(-c3ccccc3-n3c4ccccc4c4ccccc43)cccc2-n2c3ccccc3c3ccccc32)n1 ; [2H]c1c([2H])c([2H])c2c(c1[2H])c1ccccc1n2-c1cc(-n2c3ccccc3c3ccccc32)cc(-c2c(-c3cccc(-n4c5ccccc5c5ccccc54)c3)cccc2-n2c3ccccc3c3ccccc32)n1 ; [2H]c1c([2H])c([2H])c2c(c1[2H])c1ccccc1n2-c1cc(-n2c3ccccc3c3ccccc32)cc(-c2c(-c3ccc(-n4c5ccccc5c5ccccc54)cc3)cccc2-n2c3ccccc3c3ccccc32)n1]

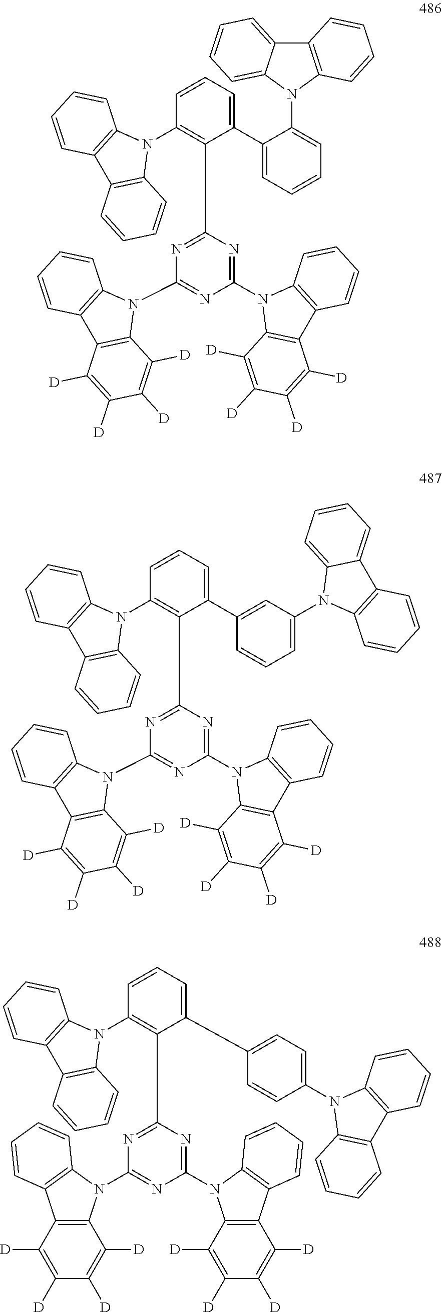 [SMILES: [2H]c1c([2H])c([2H])c2c(c1[2H])c1ccccc1n2-c1nc(-c2c(-c3ccccc3-n3c4ccccc4c4ccccc43)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2c(-c3cccc(-n4c5ccccc5c5ccccc54)c3)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2c(-c3ccc(-n4c5ccccc5c5ccccc54)cc3)cccc2-n2c3ccccc3c3ccccc32)nc(-n2c3ccccc3c3c([2H])c([2H])c([2H])c([2H])c32)n1]

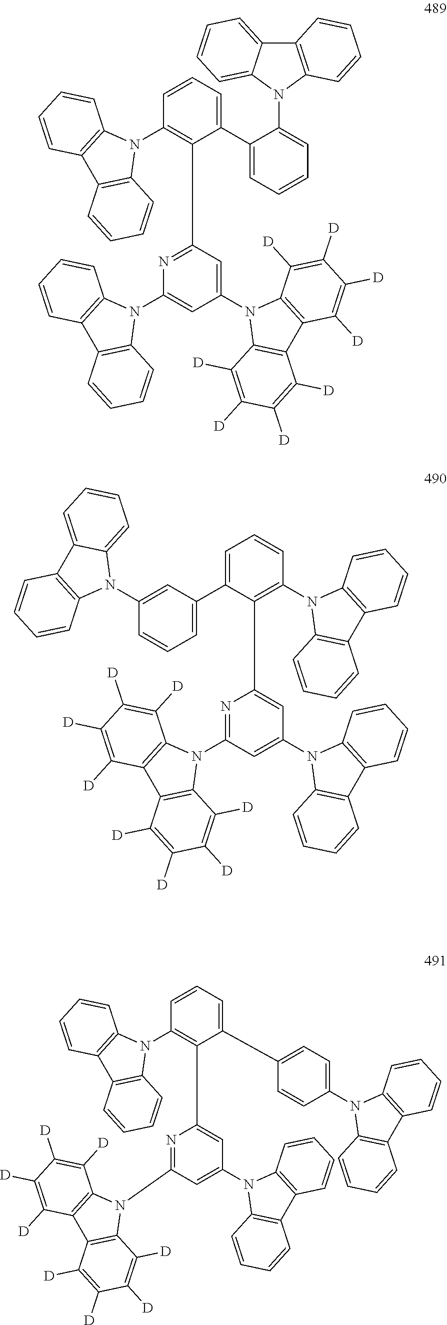 [SMILES: [2H]c1c([2H])c([2H])c2c(c1[2H])c1c([2H])c([2H])c([2H])c([2H])c1n2-c1cc(-c2c(-c3ccccc3-n3c4ccccc4c4ccccc43)cccc2-n2c3ccccc3c3ccccc32)nc(-n2c3ccccc3c3ccccc32)c1 ; [2H]c1c([2H])c([2H])c2c(c1[2H])c1c([2H])c([2H])c([2H])c([2H])c1n2-c1cc(-n2c3ccccc3c3ccccc32)cc(-c2c(-c3cccc(-n4c5ccccc5c5ccccc54)c3)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3ccccc3c3ccccc32)cc(-c2c(-c3ccc(-n4c5ccccc5c5ccccc54)cc3)cccc2-n2c3ccccc3c3ccccc32)n1]

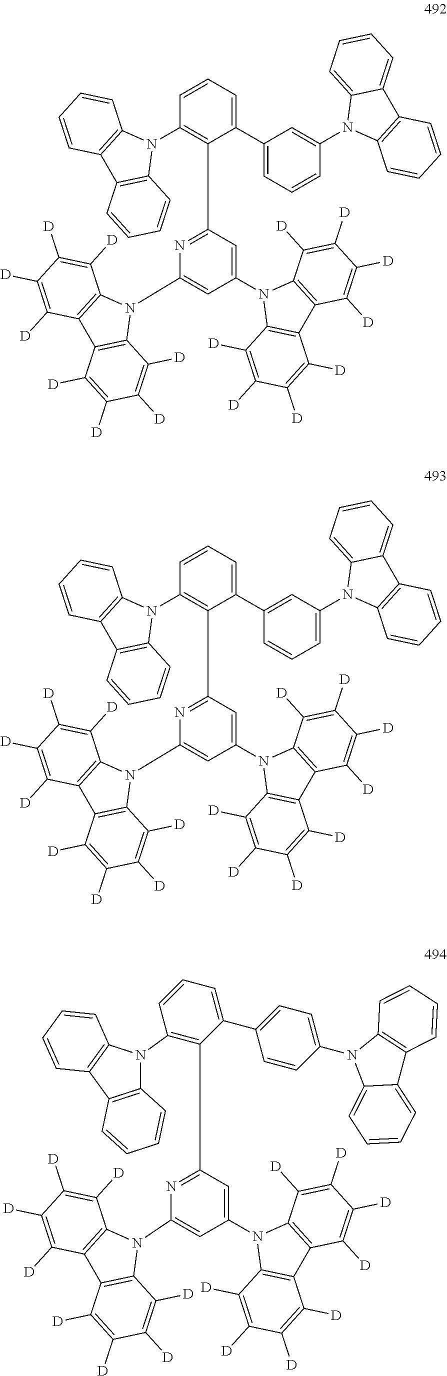

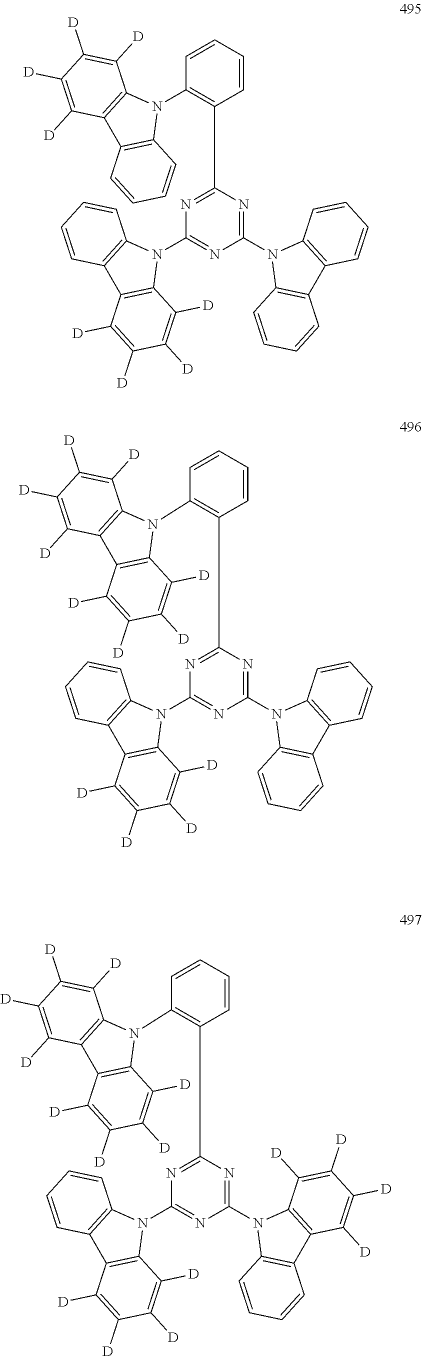 [SMILES: [2H]c1c([2H])c([2H])c2c(c1[2H])c1ccccc1n2-c1nc(-c2ccccc2-n2c3ccccc3c3c([2H])c([2H])c([2H])c([2H])c32)nc(-n2c3ccccc3c3ccccc32)n1 ; [2H]c1c([2H])c([2H])c2c(c1[2H])c1ccccc1n2-c1nc(-c2ccccc2-n2c3c([2H])c([2H])c([2H])c([2H])c3c3c([2H])c([2H])c([2H])c([2H])c32)nc(-n2c3ccccc3c3ccccc32)n1 ; [2H]c1c([2H])c([2H])c2c(c1[2H])c1ccccc1n2-c1nc(-c2ccccc2-n2c3c([2H])c([2H])c([2H])c([2H])c3c3c([2H])c([2H])c([2H])c([2H])c32)nc(-n2c3ccccc3c3c([2H])c([2H])c([2H])c([2H])c32)n1]

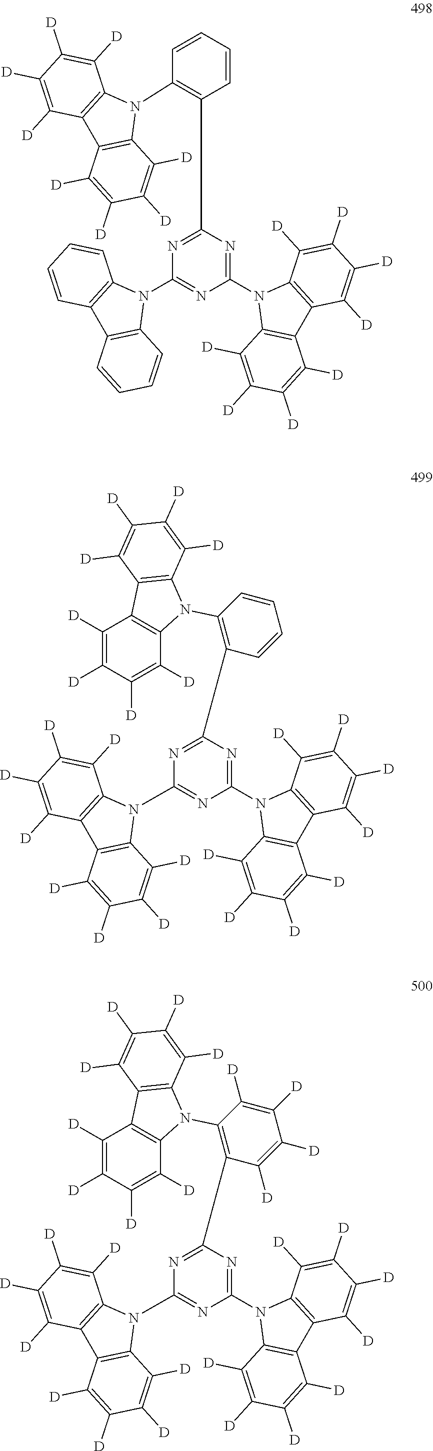

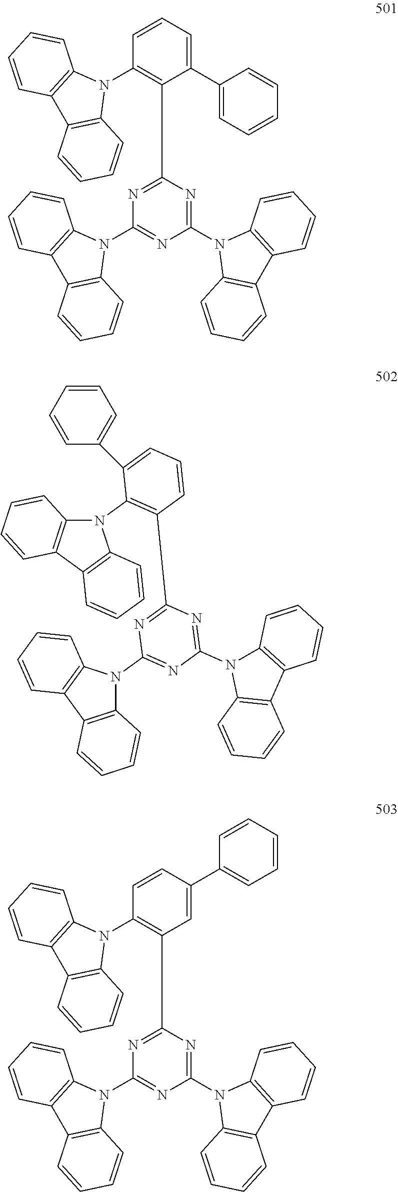 [SMILES: c1ccc(-c2cccc(-n3c4ccccc4c4ccccc43)c2-c2nc(-n3c4ccccc4c4ccccc43)nc(-n3c4ccccc4c4ccccc43)n2)cc1 ; c1ccc(-c2cccc(-c3nc(-n4c5ccccc5c5ccccc54)nc(-n4c5ccccc5c5ccccc54)n3)c2-n2c3ccccc3c3ccccc32)cc1 ; c1ccc(-c2ccc(-n3c4ccccc4c4ccccc43)c(-c3nc(-n4c5ccccc5c5ccccc54)nc(-n4c5ccccc5c5ccccc54)n3)c2)cc1]

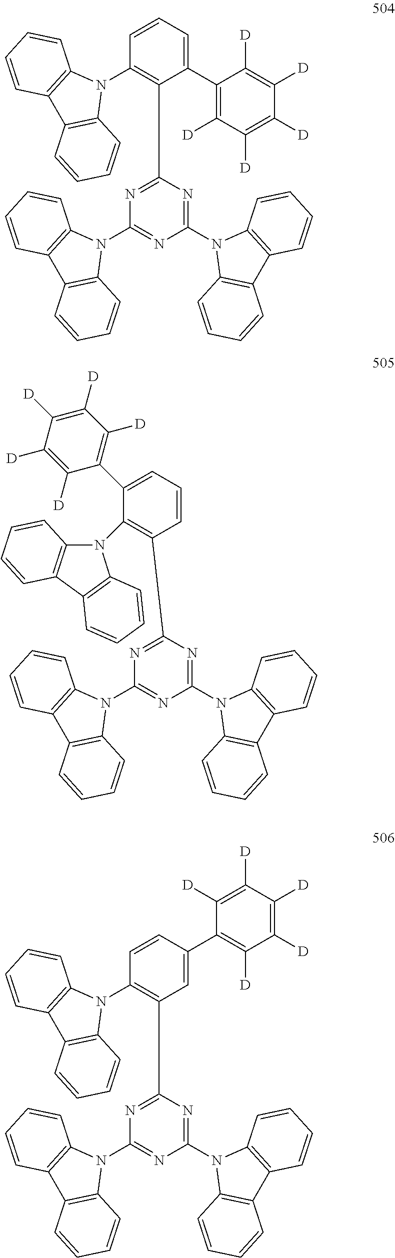

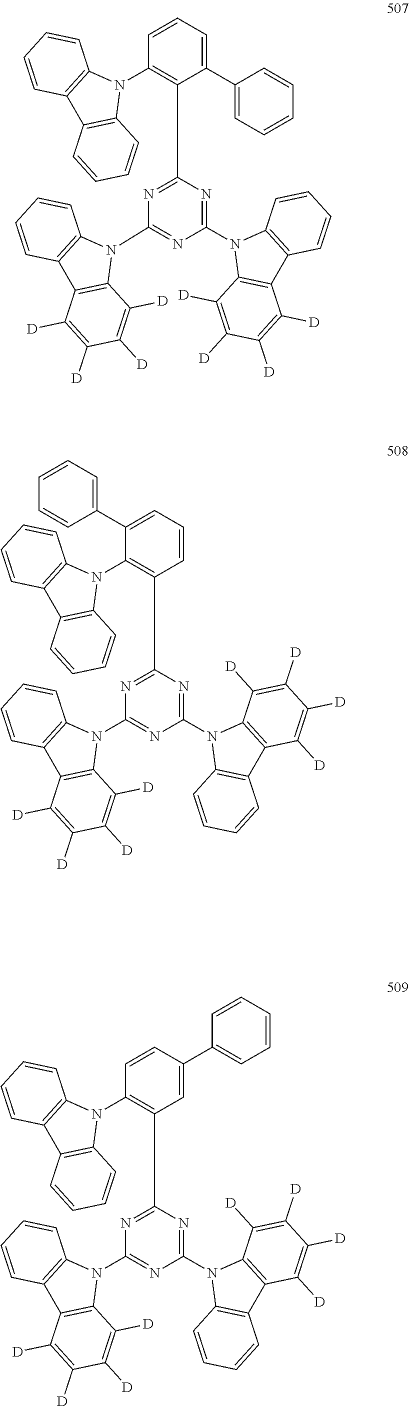 [SMILES: [2H]c1c([2H])c([2H])c2c(c1[2H])c1ccccc1n2-c1nc(-c2c(-c3ccccc3)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2cccc(-c3ccccc3)c2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2cc(-c3ccccc3)ccc2-n2c3ccccc3c3ccccc32)nc(-n2c3ccccc3c3c([2H])c([2H])c([2H])c([2H])c32)n1]

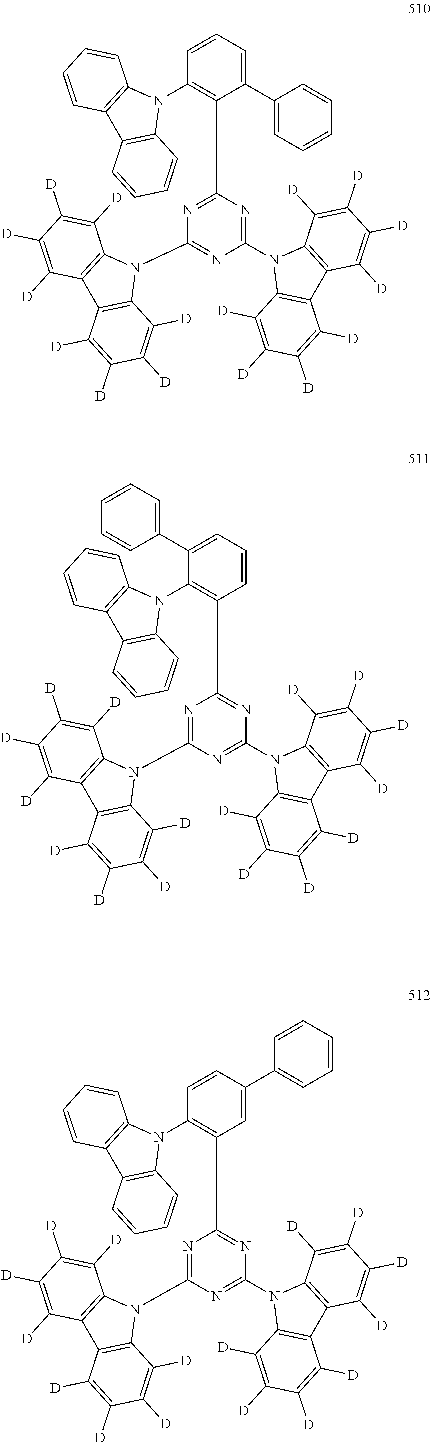

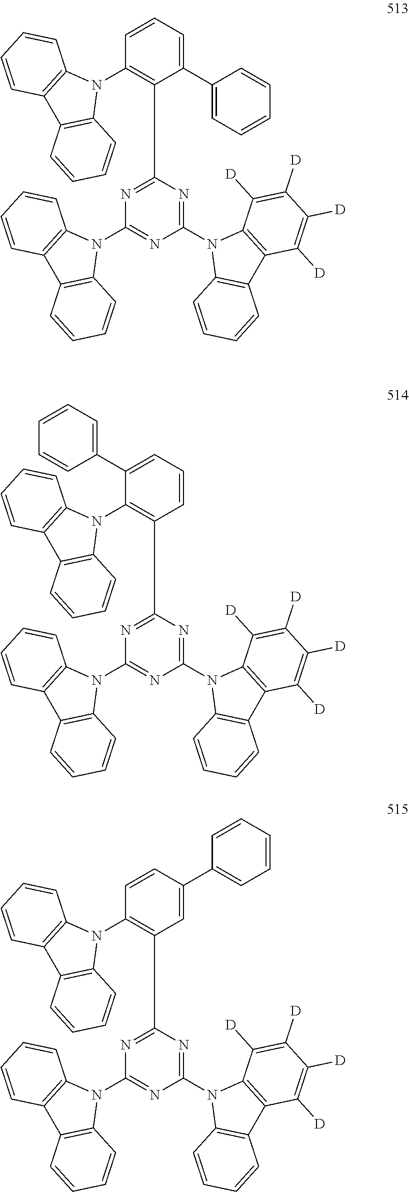

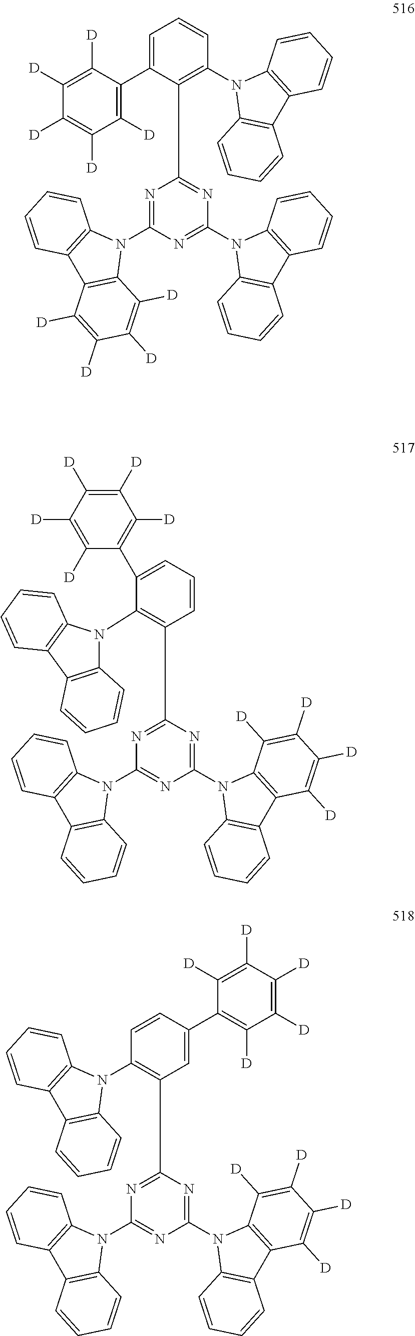 [SMILES: [2H]c1c([2H])c([2H])c(-c2cccc(-n3c4ccccc4c4ccccc43)c2-c2nc(-n3c4ccccc4c4ccccc43)nc(-n3c4ccccc4c4c([2H])c([2H])c([2H])c([2H])c43)n2)c([2H])c1[2H] ; [2H]c1c([2H])c([2H])c(-c2cccc(-c3nc(-n4c5ccccc5c5ccccc54)nc(-n4c5ccccc5c5c([2H])c([2H])c([2H])c([2H])c54)n3)c2-n2c3ccccc3c3ccccc32)c([2H])c1[2H] ; [2H]c1c([2H])c([2H])c(-c2ccc(-n3c4ccccc4c4ccccc43)c(-c3nc(-n4c5ccccc5c5ccccc54)nc(-n4c5ccccc5c5c([2H])c([2H])c([2H])c([2H])c54)n3)c2)c([2H])c1[2H]]

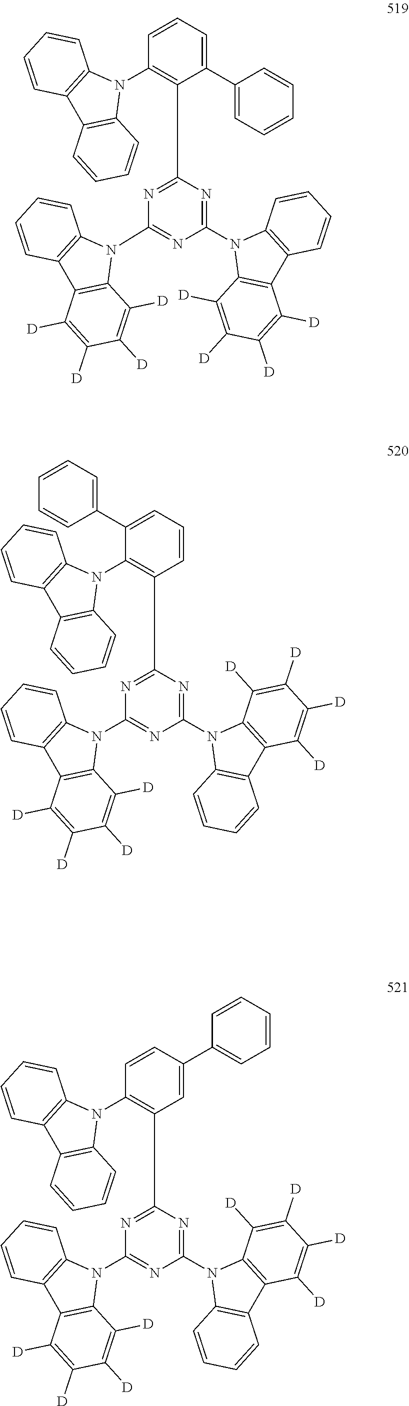 [SMILES: [2H]c1c([2H])c([2H])c2c(c1[2H])c1ccccc1n2-c1nc(-c2c(-c3ccccc3)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2cccc(-c3ccccc3)c2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2cc(-c3ccccc3)ccc2-n2c3ccccc3c3ccccc32)nc(-n2c3ccccc3c3c([2H])c([2H])c([2H])c([2H])c32)n1]

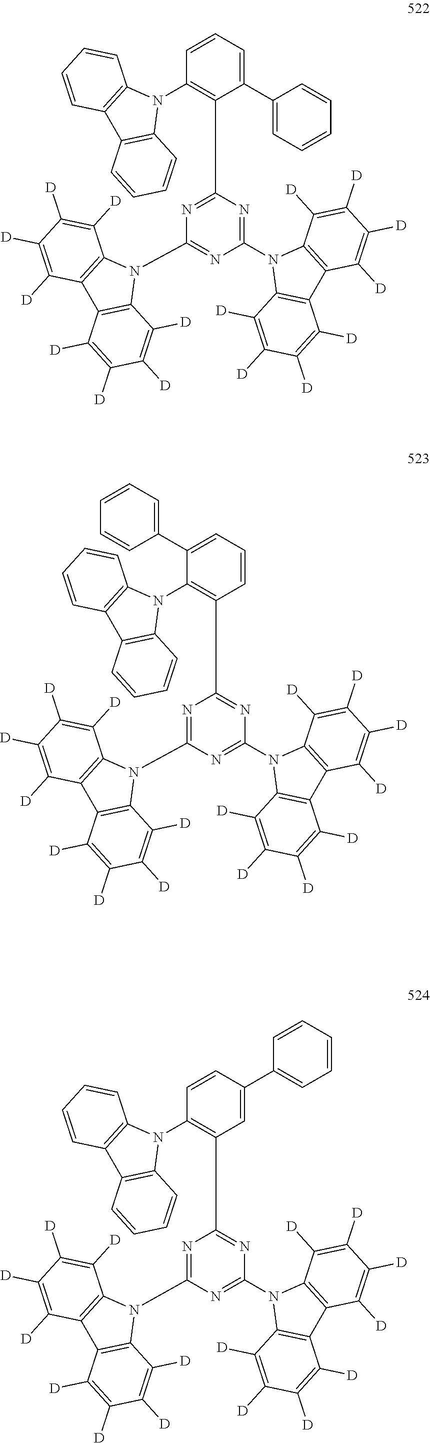

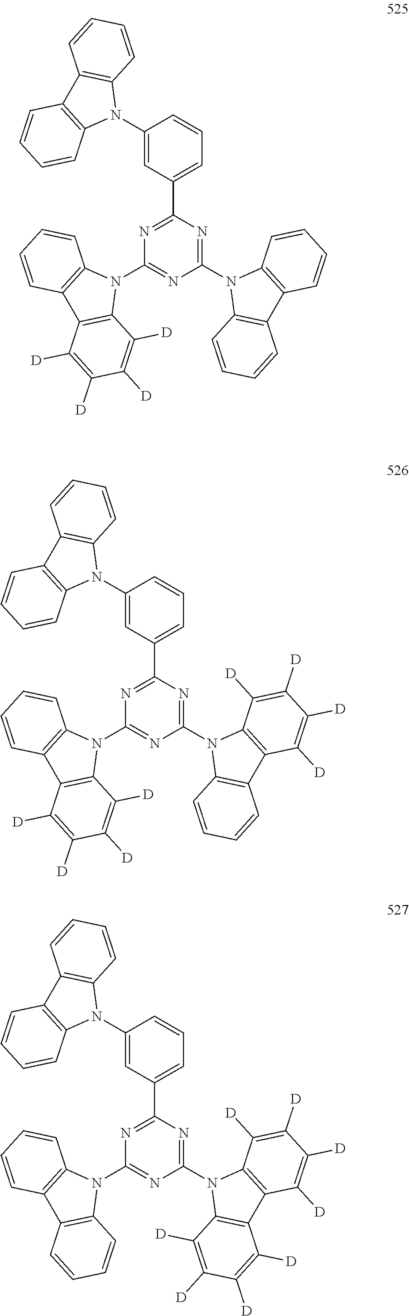 [SMILES: [2H]c1c([2H])c([2H])c2c(c1[2H])c1ccccc1n2-c1nc(-c2cccc(-n3c4ccccc4c4ccccc43)c2)nc(-n2c3ccccc3c3ccccc32)n1 ; [2H]c1c([2H])c([2H])c2c(c1[2H])c1ccccc1n2-c1nc(-c2cccc(-n3c4ccccc4c4ccccc43)c2)nc(-n2c3ccccc3c3c([2H])c([2H])c([2H])c([2H])c32)n1 ; [2H]c1c([2H])c([2H])c2c(c1[2H])c1c([2H])c([2H])c([2H])c([2H])c1n2-c1nc(-c2cccc(-n3c4ccccc4c4ccccc43)c2)nc(-n2c3ccccc3c3ccccc32)n1]

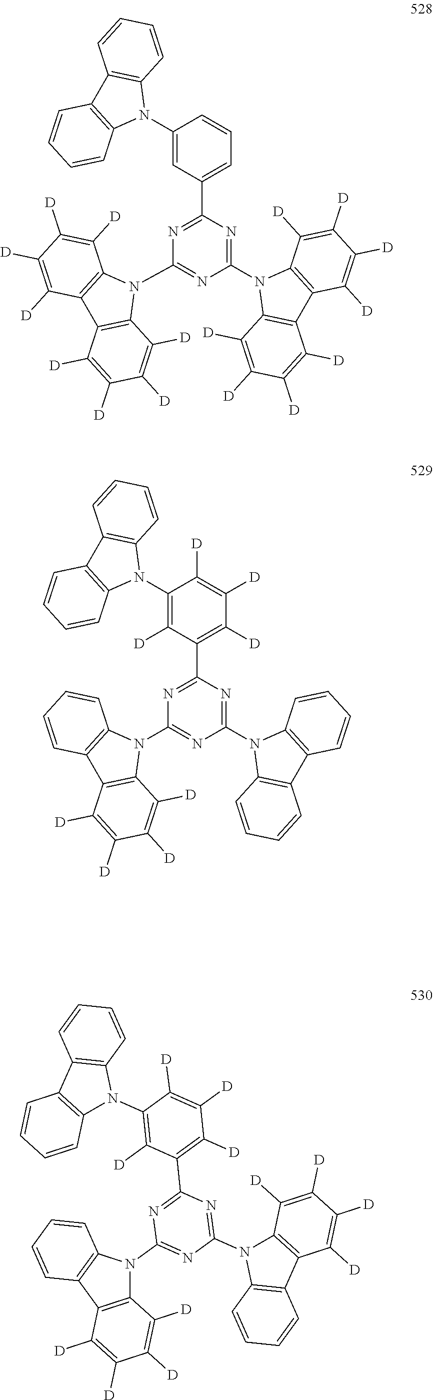 [SMILES: [2H]c1c([2H])c([2H])c2c(c1[2H])c1c([2H])c([2H])c([2H])c([2H])c1n2-c1nc(-c2cccc(-n3c4ccccc4c4ccccc43)c2)nc(-n2c3c([2H])c([2H])c([2H])c([2H])c3c3c([2H])c([2H])c([2H])c([2H])c32)n1 ; [2H]c1c([2H])c(-c2nc(-n3c4ccccc4c4ccccc43)nc(-n3c4ccccc4c4c([2H])c([2H])c([2H])c([2H])c43)n2)c([2H])c(-n2c3ccccc3c3ccccc32)c1[2H] ; [2H]c1c([2H])c(-c2nc(-n3c4ccccc4c4c([2H])c([2H])c([2H])c([2H])c43)nc(-n3c4ccccc4c4c([2H])c([2H])c([2H])c([2H])c43)n2)c([2H])c(-n2c3ccccc3c3ccccc32)c1[2H]]

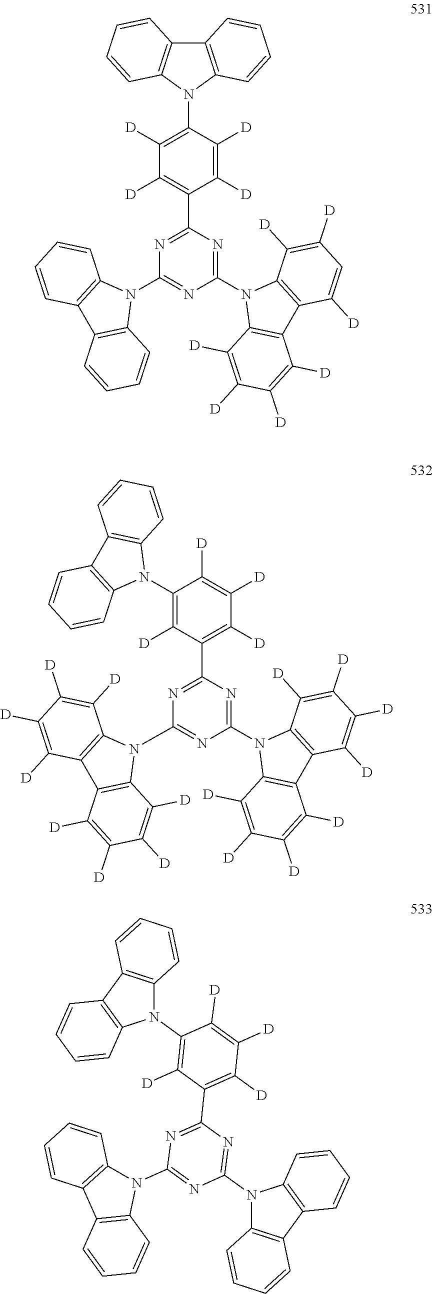 [SMILES: [2H]c1cc([2H])c2c3c([2H])c([2H])c([2H])c([2H])c3n(-c3nc(-c4c([2H])c([2H])c(-n5c6ccccc6c6ccccc65)c([2H])c4[2H])nc(-n4c5ccccc5c5ccccc54)n3)c2c1[2H] ; [2H]c1c([2H])c(-c2nc(-n3c4c([2H])c([2H])c([2H])c([2H])c4c4c([2H])c([2H])c([2H])c([2H])c43)nc(-n3c4c([2H])c([2H])c([2H])c([2H])c4c4c([2H])c([2H])c([2H])c([2H])c43)n2)c([2H])c(-n2c3ccccc3c3ccccc32)c1[2H] ; [2H]c1c([2H])c(-c2nc(-n3c4ccccc4c4ccccc43)nc(-n3c4ccccc4c4ccccc43)n2)c([2H])c(-n2c3ccccc3c3ccccc32)c1[2H]]

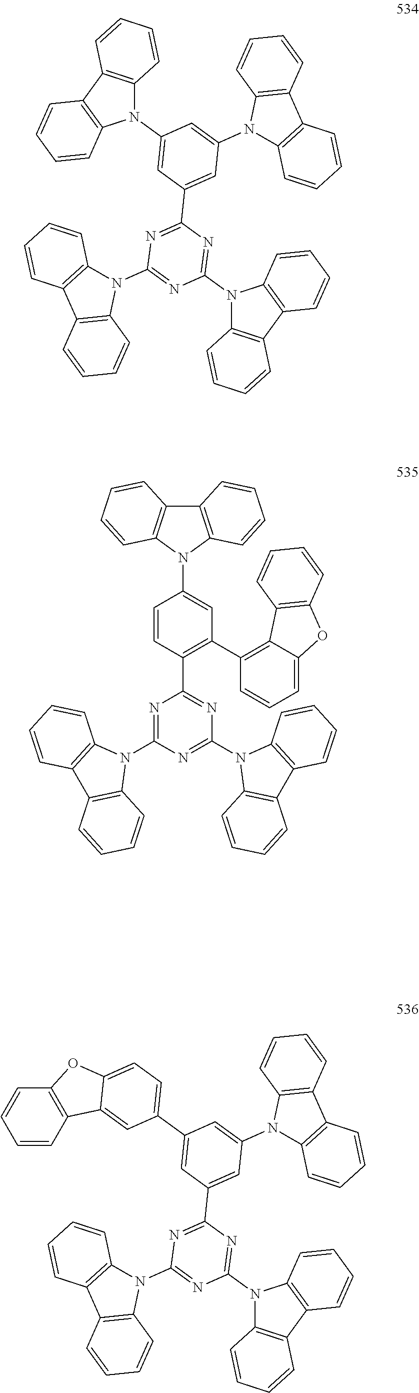 [SMILES: c1ccc2c(c1)c1ccccc1n2-c1cc(-c2nc(-n3c4ccccc4c4ccccc43)nc(-n3c4ccccc4c4ccccc43)n2)cc(-n2c3ccccc3c3ccccc32)c1 ; c1ccc2c(c1)oc1cccc(-c3cc(-n4c5ccccc5c5ccccc54)ccc3-c3nc(-n4c5ccccc5c5ccccc54)nc(-n4c5ccccc5c5ccccc54)n3)c12 ; c1ccc2c(c1)oc1ccc(-c3cc(-c4nc(-n5c6ccccc6c6ccccc65)nc(-n5c6ccccc6c6ccccc65)n4)cc(-n4c5ccccc5c5ccccc54)c3)cc12]

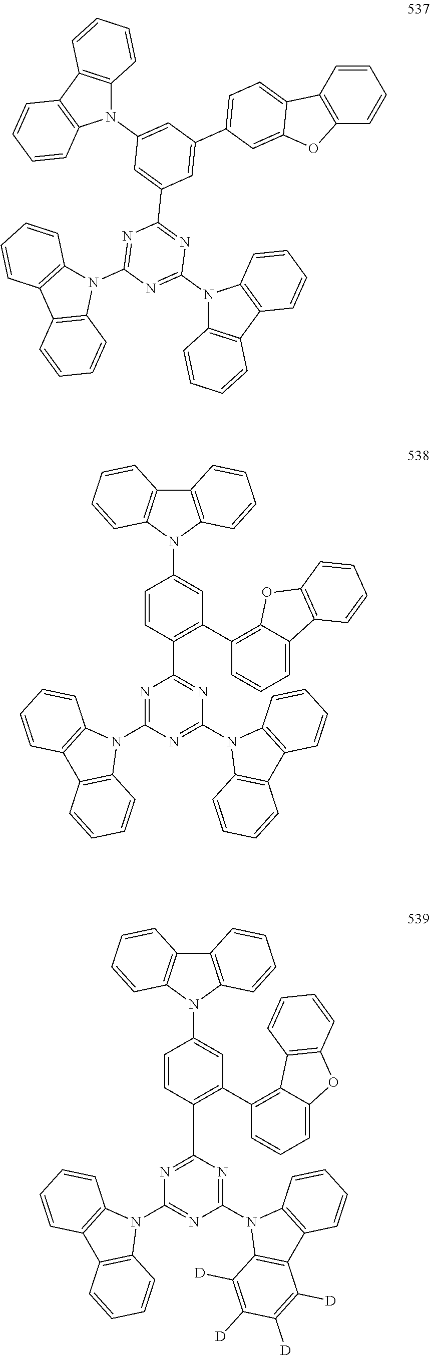 [SMILES: c1ccc2c(c1)oc1cc(-c3cc(-c4nc(-n5c6ccccc6c6ccccc65)nc(-n5c6ccccc6c6ccccc65)n4)cc(-n4c5ccccc5c5ccccc54)c3)ccc12 ; c1ccc2c(c1)oc1c(-c3cc(-n4c5ccccc5c5ccccc54)ccc3-c3nc(-n4c5ccccc5c5ccccc54)nc(-n4c5ccccc5c5ccccc54)n3)cccc12 ; [2H]c1c([2H])c([2H])c2c(c1[2H])c1ccccc1n2-c1nc(-c2ccc(-n3c4ccccc4c4ccccc43)cc2-c2cccc3oc4ccccc4c23)nc(-n2c3ccccc3c3ccccc32)n1]

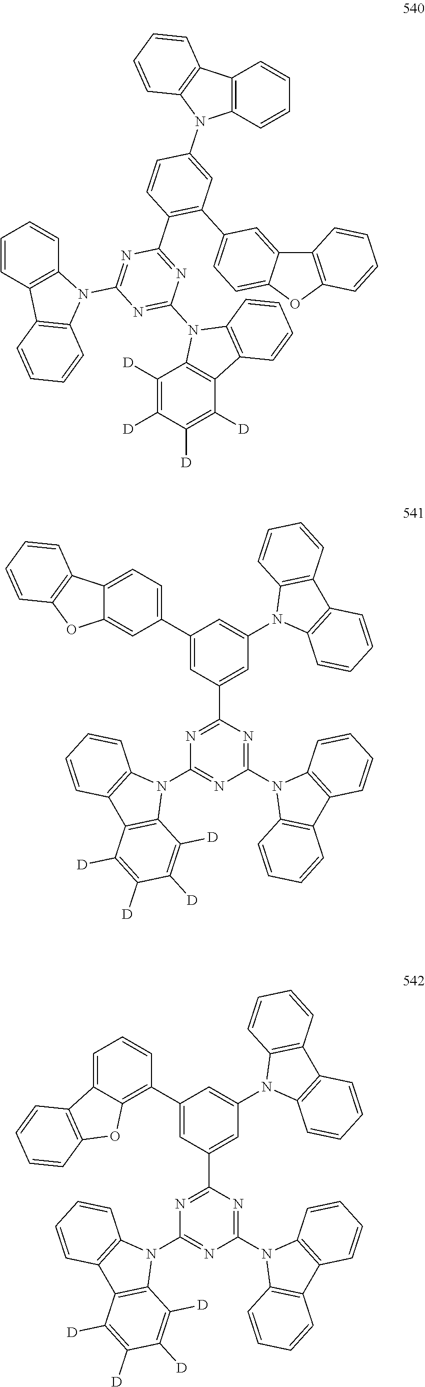 [SMILES: [2H]c1c([2H])c([2H])c2c(c1[2H])c1ccccc1n2-c1nc(-c2ccc(-n3c4ccccc4c4ccccc43)cc2-c2ccc3oc4ccccc4c3c2)nc(-n2c3ccccc3c3ccccc32)n1 ; [2H]c1c([2H])c([2H])c2c(c1[2H])c1ccccc1n2-c1nc(-c2cc(-c3ccc4c(c3)oc3ccccc34)cc(-n3c4ccccc4c4ccccc43)c2)nc(-n2c3ccccc3c3ccccc32)n1 ; [2H]c1c([2H])c([2H])c2c(c1[2H])c1ccccc1n2-c1nc(-c2cc(-c3cccc4c3oc3ccccc34)cc(-n3c4ccccc4c4ccccc43)c2)nc(-n2c3ccccc3c3ccccc32)n1]

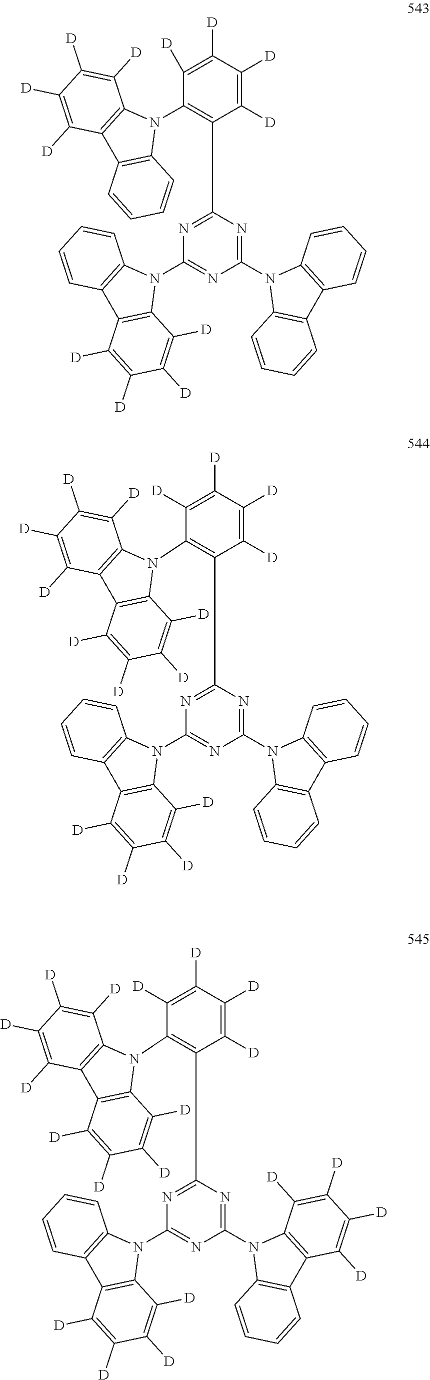

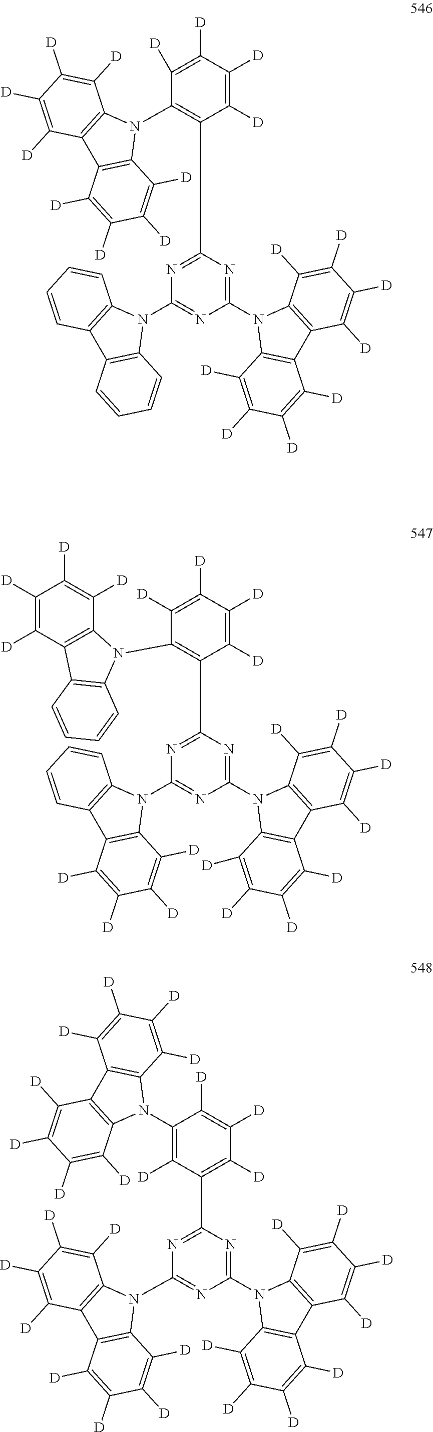

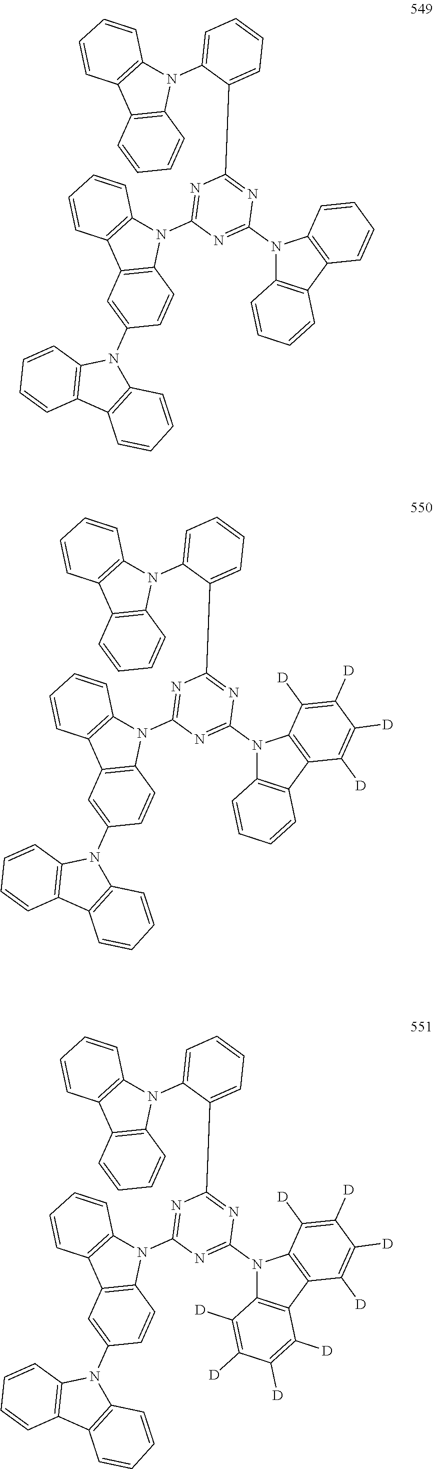 [SMILES: c1ccc(-n2c3ccccc3c3ccccc32)c(-c2nc(-n3c4ccccc4c4ccccc43)nc(-n3c4ccccc4c4cc(-n5c6ccccc6c6ccccc65)ccc43)n2)c1 ; [2H]c1c([2H])c([2H])c2c(c1[2H])c1ccccc1n2-c1nc(-c2ccccc2-n2c3ccccc3c3ccccc32)nc(-n2c3ccccc3c3cc(-n4c5ccccc5c5ccccc54)ccc32)n1 ; [2H]c1c([2H])c([2H])c2c(c1[2H])c1c([2H])c([2H])c([2H])c([2H])c1n2-c1nc(-c2ccccc2-n2c3ccccc3c3ccccc32)nc(-n2c3ccccc3c3cc(-n4c5ccccc5c5ccccc54)ccc32)n1]

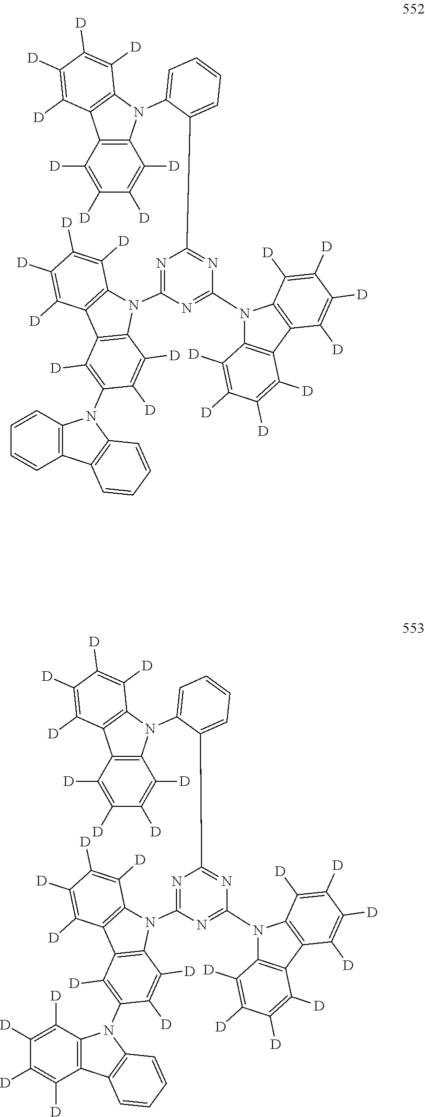

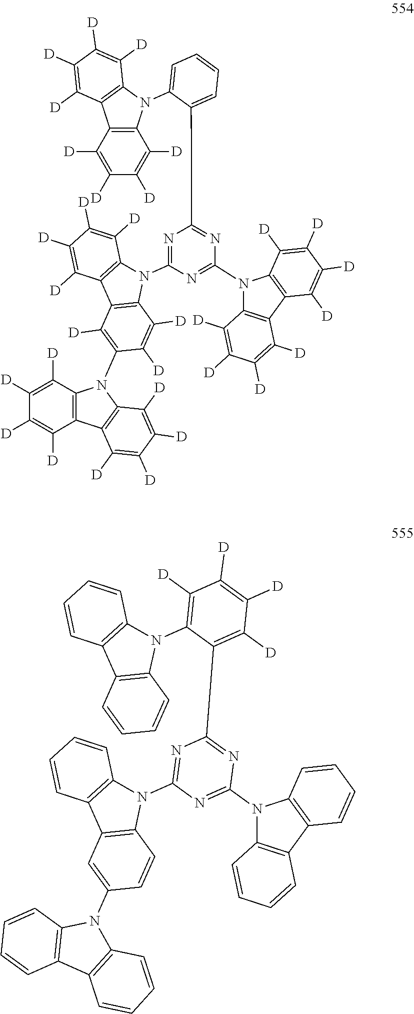 [SMILES: [2H]c1c([2H])c([2H])c2c(c1[2H])c1c([2H])c([2H])c([2H])c([2H])c1n2-c1nc(-c2ccccc2-n2c3c([2H])c([2H])c([2H])c([2H])c3c3c([2H])c([2H])c([2H])c([2H])c32)nc(-n2c3c([2H])c([2H])c([2H])c([2H])c3c3c([2H])c(-n4c5c([2H])c([2H])c([2H])c([2H])c5c5c([2H])c([2H])c([2H])c([2H])c54)c([2H])c([2H])c32)n1 ; [2H]c1c([2H])c([2H])c(-n2c3ccccc3c3ccccc32)c(-c2nc(-n3c4ccccc4c4ccccc43)nc(-n3c4ccccc4c4cc(-n5c6ccccc6c6ccccc65)ccc43)n2)c1[2H]]

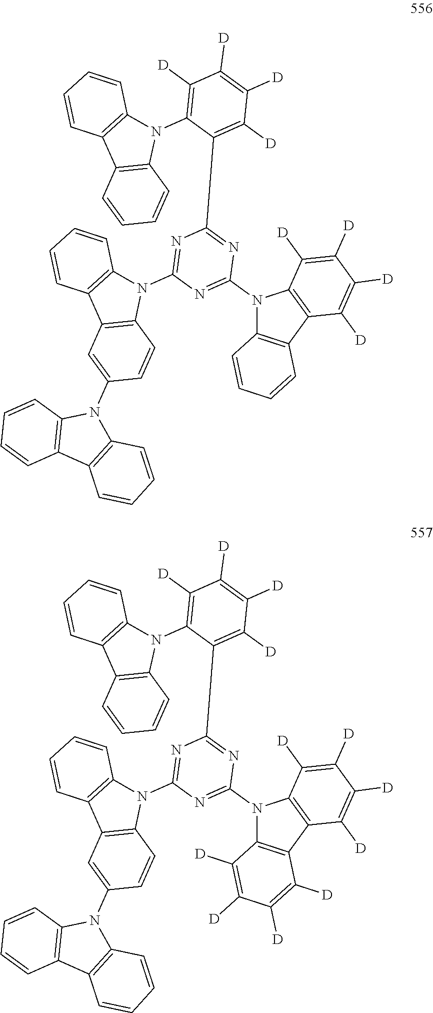 [SMILES: [2H]c1c([2H])c([2H])c(-n2c3ccccc3c3ccccc32)c(-c2nc(-n3c4ccccc4c4cc(-n5c6ccccc6c6ccccc65)ccc43)nc(-n3c4ccccc4c4c([2H])c([2H])c([2H])c([2H])c43)n2)c1[2H] ; [2H]c1c([2H])c([2H])c(-n2c3ccccc3c3ccccc32)c(-c2nc(-n3c4ccccc4c4cc(-n5c6ccccc6c6ccccc65)ccc43)nc(-n3c4c([2H])c([2H])c([2H])c([2H])c4c4c([2H])c([2H])c([2H])c([2H])c43)n2)c1[2H]]

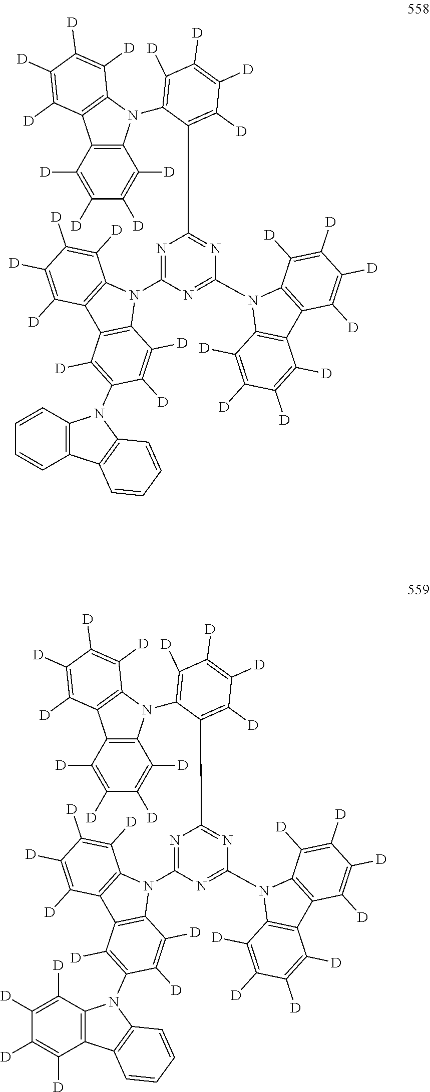

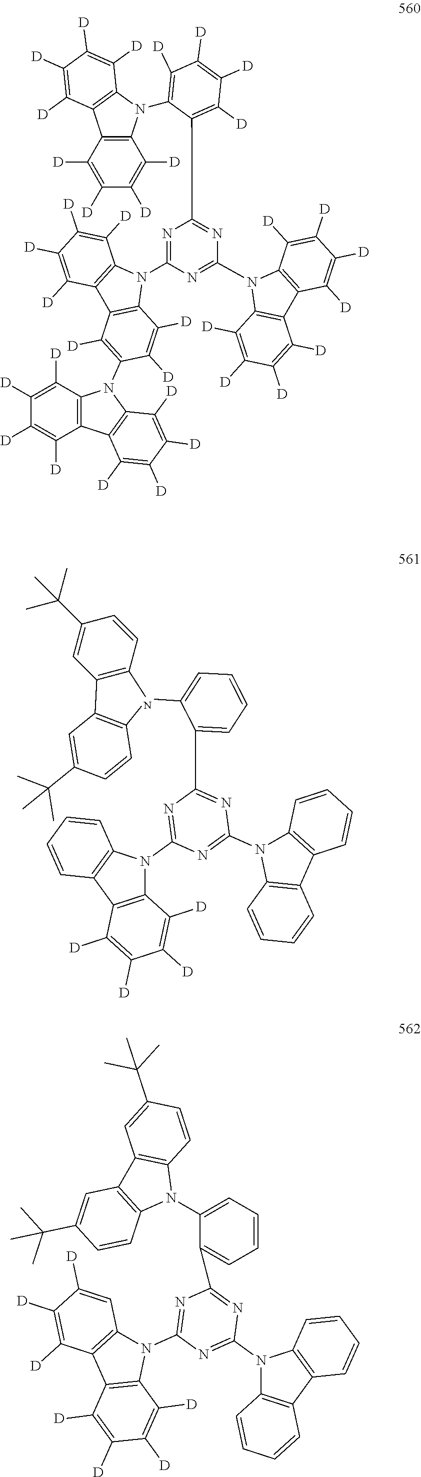

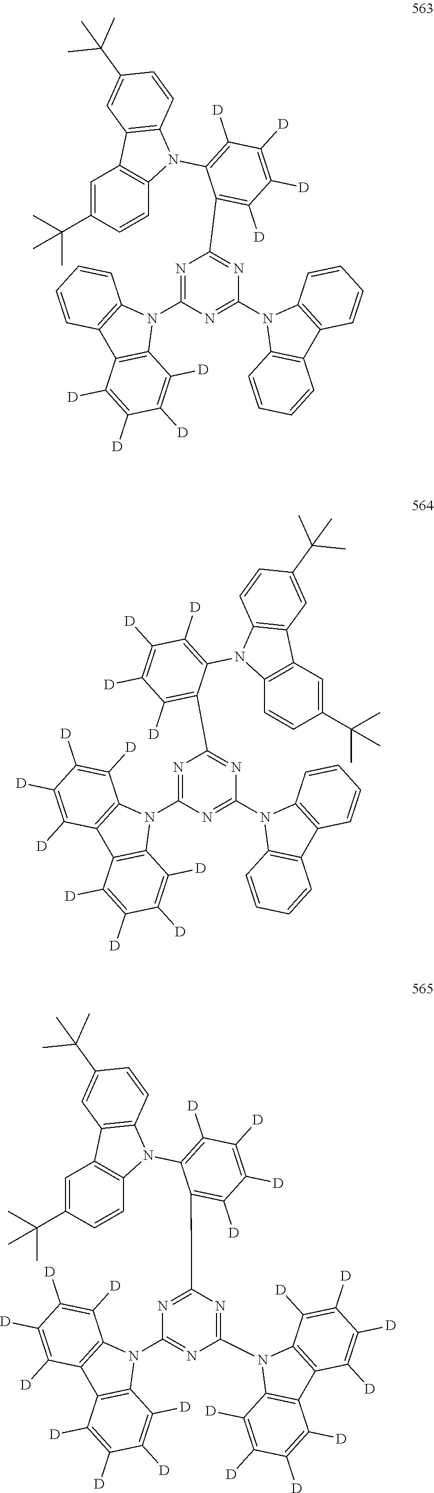

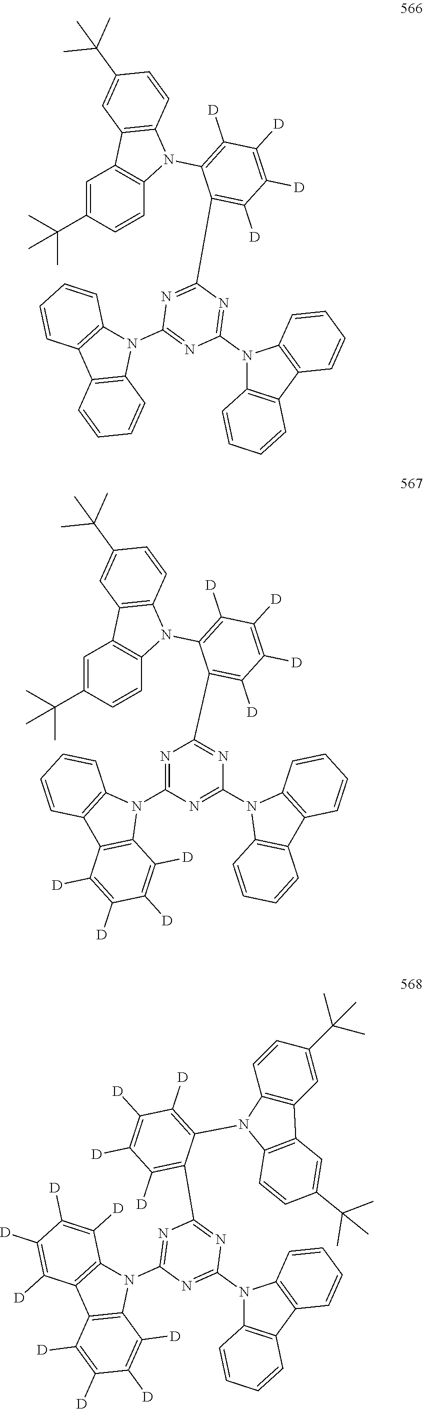 [SMILES: [2H]c1c([2H])c([2H])c(-n2c3ccc(C(C)(C)C)cc3c3cc(C(C)(C)C)ccc32)c(-c2nc(-n3c4ccccc4c4ccccc43)nc(-n3c4ccccc4c4ccccc43)n2)c1[2H] ; [2H]c1c([2H])c([2H])c(-n2c3ccc(C(C)(C)C)cc3c3cc(C(C)(C)C)ccc32)c(-c2nc(-n3c4ccccc4c4ccccc43)nc(-n3c4ccccc4c4c([2H])c([2H])c([2H])c([2H])c43)n2)c1[2H] ; [2H]c1c([2H])c([2H])c(-n2c3ccc(C(C)(C)C)cc3c3cc(C(C)(C)C)ccc32)c(-c2nc(-n3c4ccccc4c4ccccc43)nc(-n3c4c([2H])c([2H])c([2H])c([2H])c4c4c([2H])c([2H])c([2H])c([2H])c43)n2)c1[2H]]

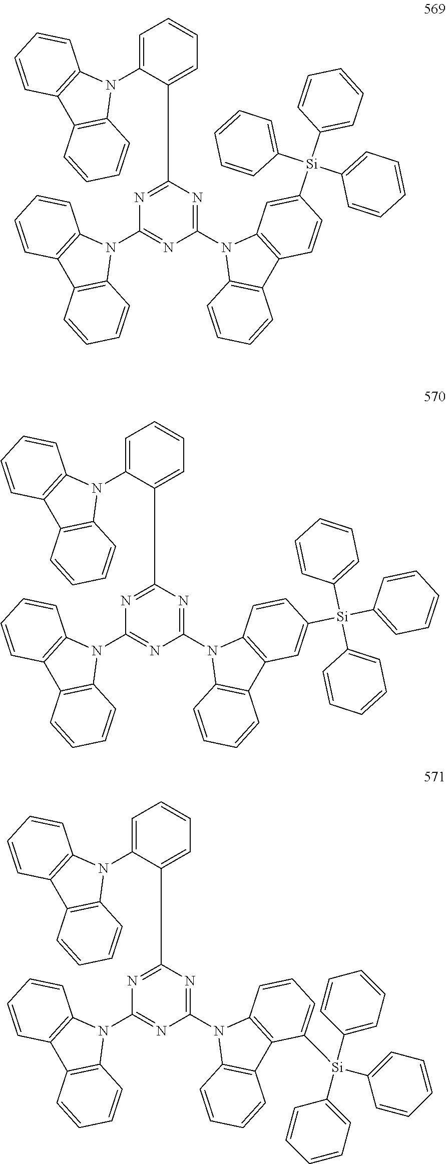 [SMILES: c1ccc([Si](c2ccccc2)(c2ccccc2)c2ccc3c4ccccc4n(-c4nc(-c5ccccc5-n5c6ccccc6c6ccccc65)nc(-n5c6ccccc6c6ccccc65)n4)c3c2)cc1 ; c1ccc([Si](c2ccccc2)(c2ccccc2)c2ccc3c(c2)c2ccccc2n3-c2nc(-c3ccccc3-n3c4ccccc4c4ccccc43)nc(-n3c4ccccc4c4ccccc43)n2)cc1 ; c1ccc([Si](c2ccccc2)(c2ccccc2)c2cccc3c2c2ccccc2n3-c2nc(-c3ccccc3-n3c4ccccc4c4ccccc43)nc(-n3c4ccccc4c4ccccc43)n2)cc1]

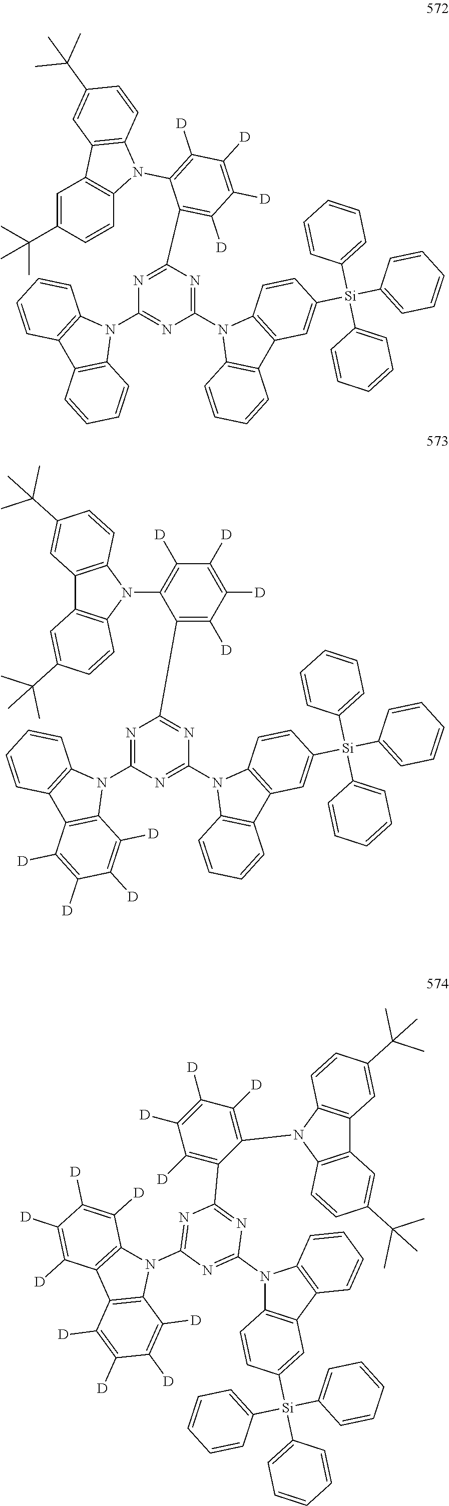

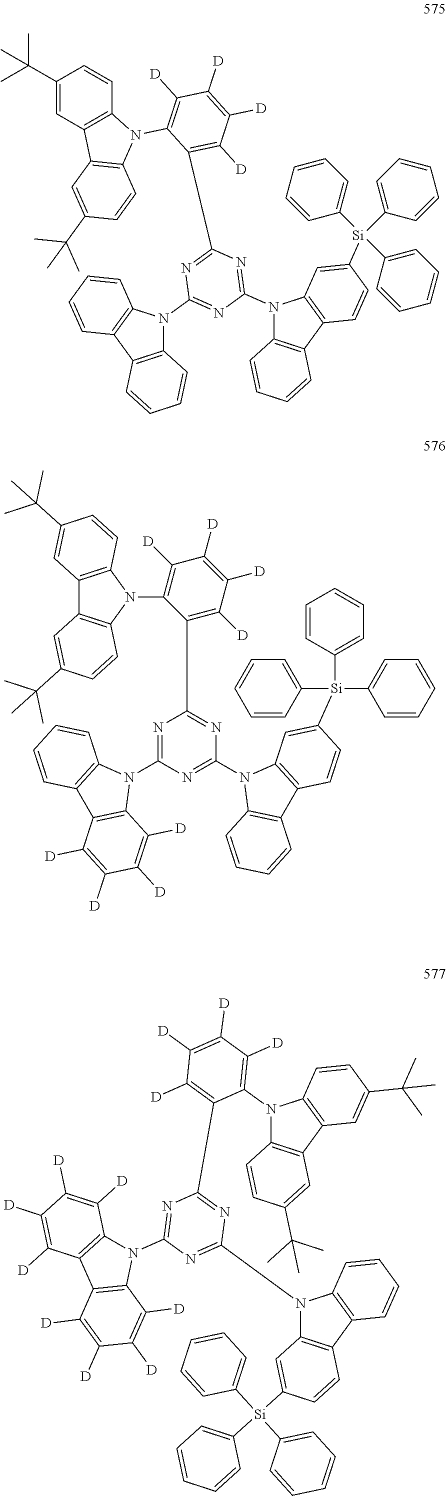

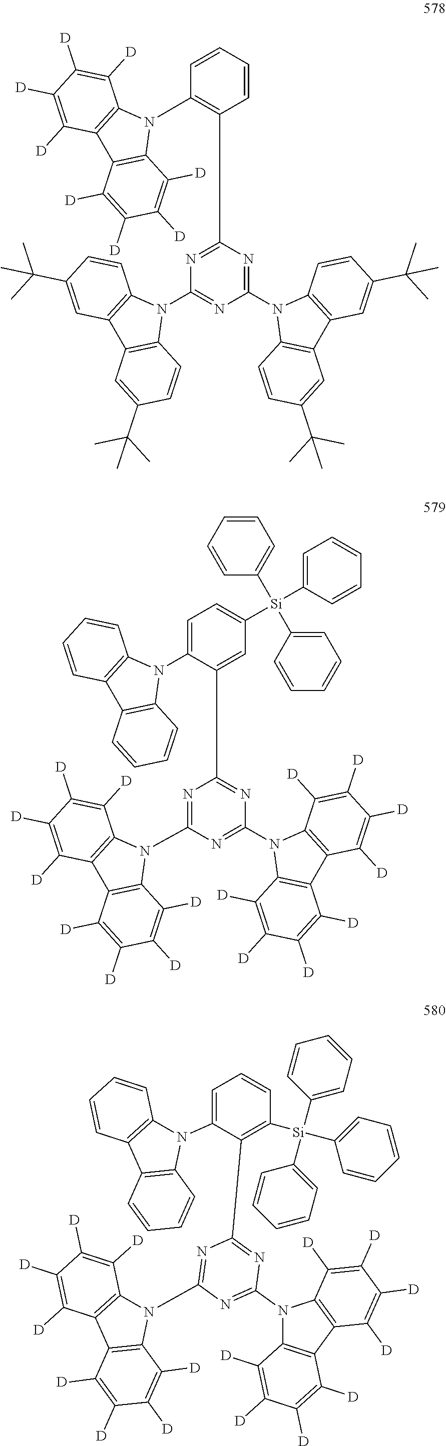

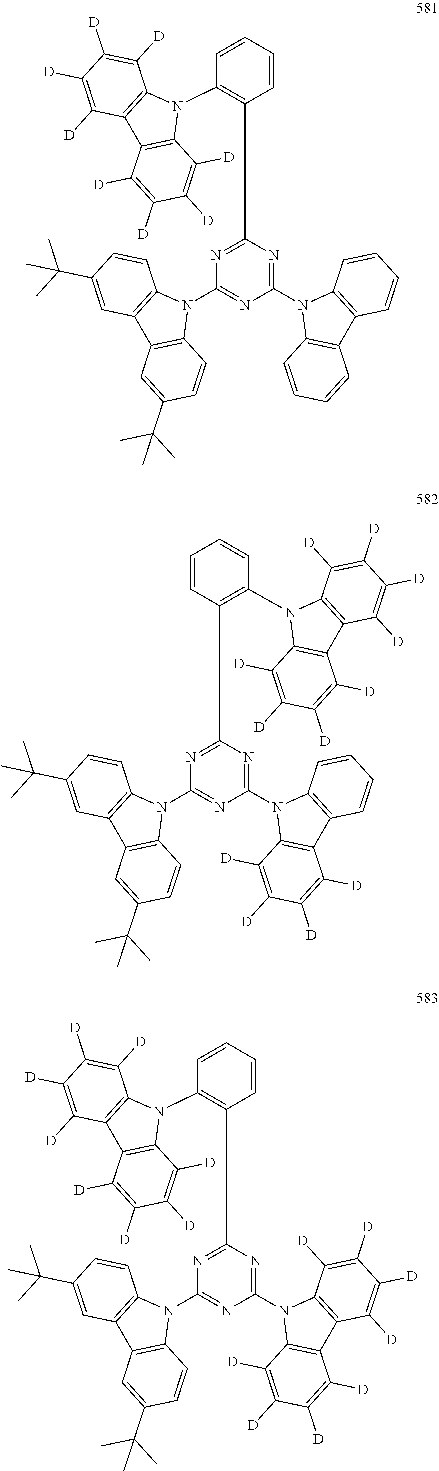

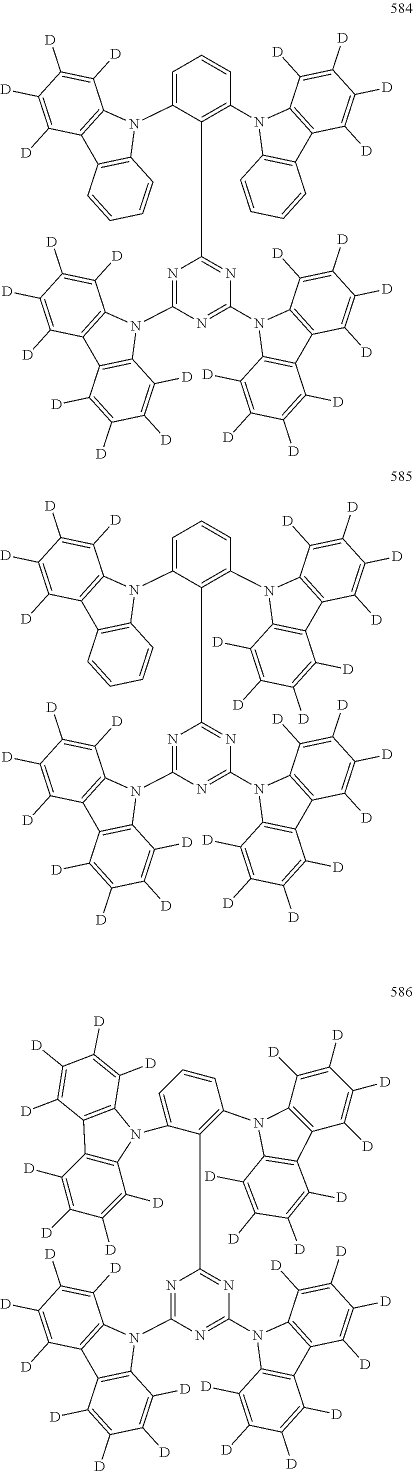

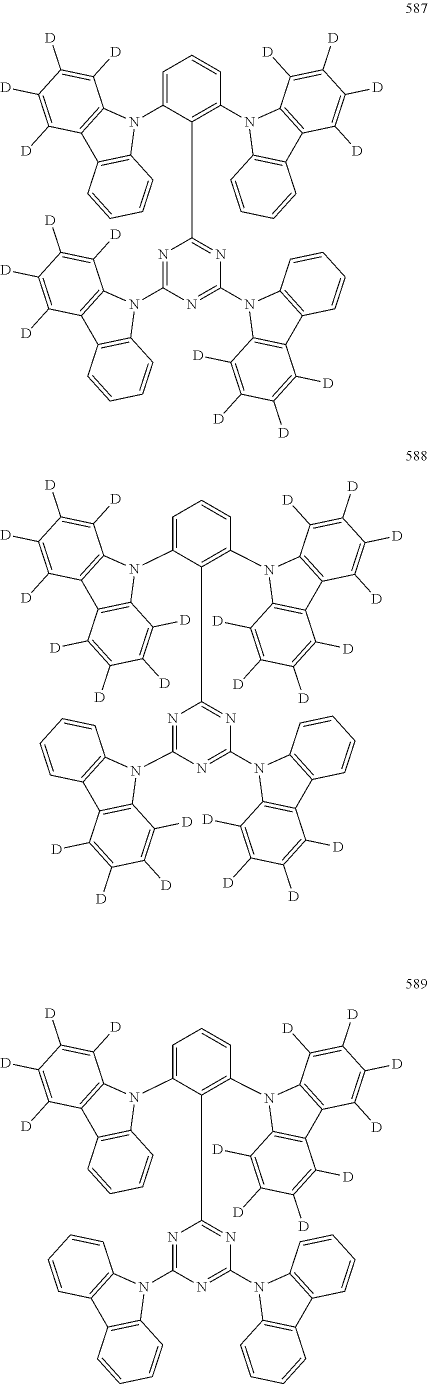

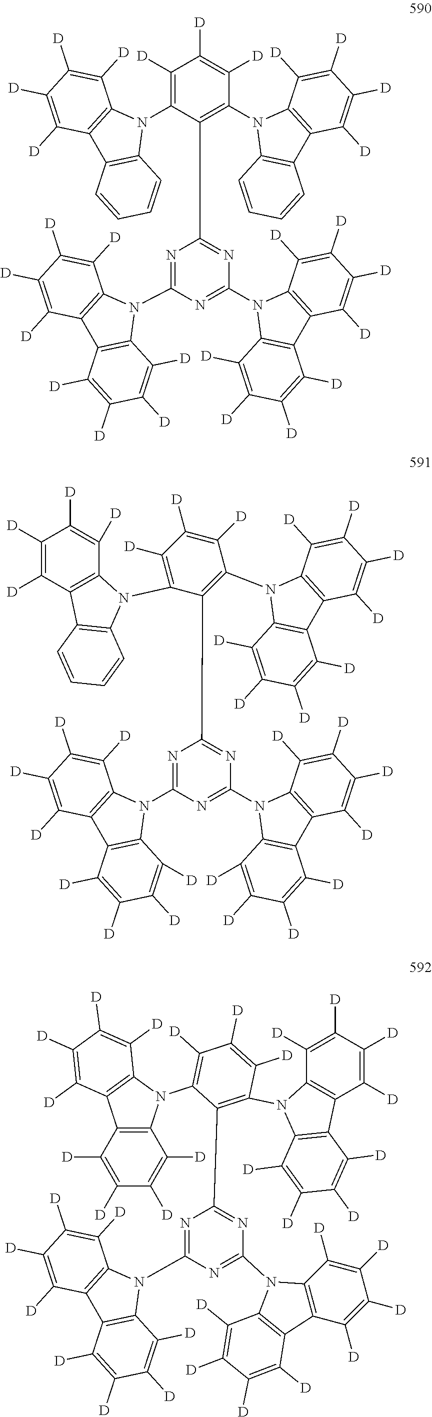

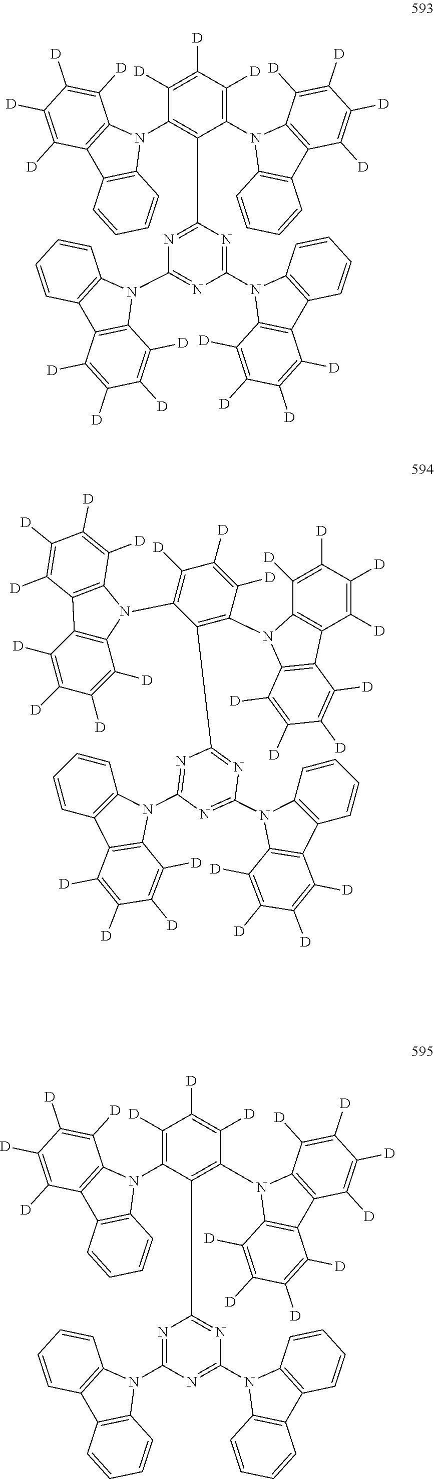

The nitrogen-containing compound of an embodiment may include a nitrogen-containing core portion including at least one nitrogen atom as a ring-forming atom, and may include first to third substituents directly or indirectly bonded to the core portion. In an embodiment, the first to third substituents may be carbazole groups. In the nitrogen-containing compound of an embodiment, two of the three carbazole groups may be directly bonded to the core portion, and the other one carbazole group may be bonded to the core portion through a linker of a benzene ring. The nitrogen-containing compound according to an embodiment may include a fourth substituent on at least one of the three carbazole groups and the linker of a benzene ring. The fourth substituent may be a deuterium atom, an unsubstituted carbazole group, a deuterium atom-substituted or unsubstituted phenyl group, an unsubstituted dibenzofuran group, an unsubstituted dibenzothiophene group, or an unsubstituted 9-phenylcarbazole group.

In an embodiment, the emission layer EML may include a host and a dopant. The nitrogen-containing compound of an embodiment may be used as a host material of the emission layer EML. The nitrogen-containing compound of an embodiment includes the first to fourth substituents to have a relatively large volume, and may thus prevent or reduce formation of an exciplex by the nitrogen-containing compound and a dopant. In an embodiment, the light emitting element ED including the nitrogen-containing compound may have reduced driving voltage and increased efficiency and lifespan. However, the embodiment of the present disclosure is not limited thereto, and the nitrogen-containing compound may be used as a host material for fluorescence emission.

For example, the emission layer EML may include a single host and a single dopant. In one or more embodiments, the emission layer EML may include two or more hosts, sensitizers, and dopants. For example, the emission layer EML may include a hole transporting host and an electron transporting host. The emission layer EML of an embodiment may include the nitrogen-containing compound of an embodiment as a host. In one or more embodiments, the nitrogen-containing compound of an embodiment may be used as an electron transporting host material.

The emission layer EML may include a phosphorescent sensitizer or a thermally activated delayed fluorescence (TADF) sensitizer as a sensitizer. For example, the phosphorescent sensitizer may be a material including a metal complex. The thermally activated delayed fluorescence sensitizer may include a compound represented by Formula F-c which will be further described herein below. However, this is presented as an example, and the phosphorescent sensitizer and the thermally activated delayed fluorescence sensitizer are not limited thereto.

When the emission layer EML includes the hole transporting host, the electron transporting host, a sensitizer, and a dopant, the hole transporting host and the electron transporting host may form an exciplex, and energy may be transferred from the exciplex to the sensitizer and from the sensitizer to the dopant, thereby emitting light. However, this is presented as an example, and materials included in the emission layer EML are not limited thereto.

In the emission layer EML, the hole transporting host and the electron transporting host may form an exciplex. In this case, the triplet energy of the exciplex formed by the hole transporting host and the electron transporting host corresponds to a difference between Lowest Unoccupied Molecular Orbital (LUMO) energy level of the electron transporting host and Highest Occupied Molecular Orbital (HOMO) energy level of the hole transporting host.

For example, the triplet energy level (T1) of the exciplex formed by the hole transporting host and the electron transporting host may have an absolute value of about 2.4 eV to about 3.0 eV. In addition, the triplet energy of the exciplex may have a value smaller than the energy band gap of each host material. The exciplex may have a triplet energy of 3.0 eV or less, which is an energy band gap between the hole transporting host and the electron transporting host. However, this is presented as an example, and the embodiment of the present disclosure is not limited thereto.

The emission layer EML of an embodiment may further include the second compound represented by Formula HT below. The second compound may be used as a hole transporting host material.

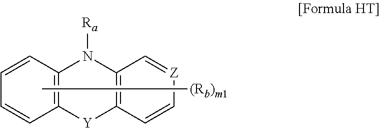

In Formula HT, m1 may be an integer of 0 to 7. When m1 is an integer of 2 or greater, a plurality of Rb's may all be the same or at least one may be different from the others. Rₐ and Rb may each independently be a hydrogen atom, a deuterium atom, a cyano group, a substituted or unsubstituted aryl group having 6 to 60 ring-forming carbon atoms, or a substituted or unsubstituted heteroaryl group having 2 to 60 ring-forming carbon atoms, or bonded to an adjacent group to form an aromatic ring. For example, Rₐ may be a substituted phenyl group, an unsubstituted dibenzofuran group, or a substituted fluorenyl group. Rb may be a hydrogen atom, a deuterium atom, a substituted or unsubstituted phenyl group, a substituted or unsubstituted carbazole group, or a substituted or unsubstituted fluorenyl group. In addition, two adjacent Rb's may be bonded to each other to form a substituted or unsubstituted heterocycle.

In Formula HT, Y may be a direct linkage, CR_{y1}R_{y2}, or SiR_{y3}R_{y4}. For example, when Y is a direct linkage, the second compound represented by Formula HT may include a carbazole skeleton. R_{y1} to R_{y4} may each independently be a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted aryl group having 6 to 60 ring-forming carbon atoms, or a substituted or unsubstituted heteroaryl group having 2 to 60 ring-forming carbon atoms. For example, R_{y1} to R_{y4} may each independently be a methyl group or a phenyl group.

In Formula HT, Z may be CR_{z} or a nitrogen atom (N). For example, when Y is a direct linkage and Z is a CRY, Formula HT may include a carbazole skeleton. In addition, when Y is a direct linkage and Z is a nitrogen atom, Formula HT may include a pyridoindole skeleton. R_{z} may be a hydrogen atom or a deuterium atom.

The second compound may be represented by any one selected from among compounds of Compound Group 2 below. The light emitting element ED according to an embodiment may include any one selected from among compounds of Compound Group 2 below. In Compound Group 2 below, “D” is a deuterium atom.

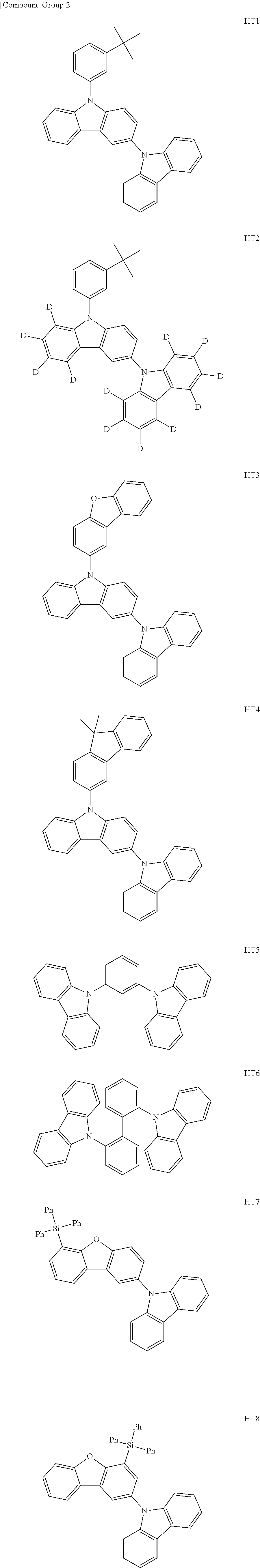

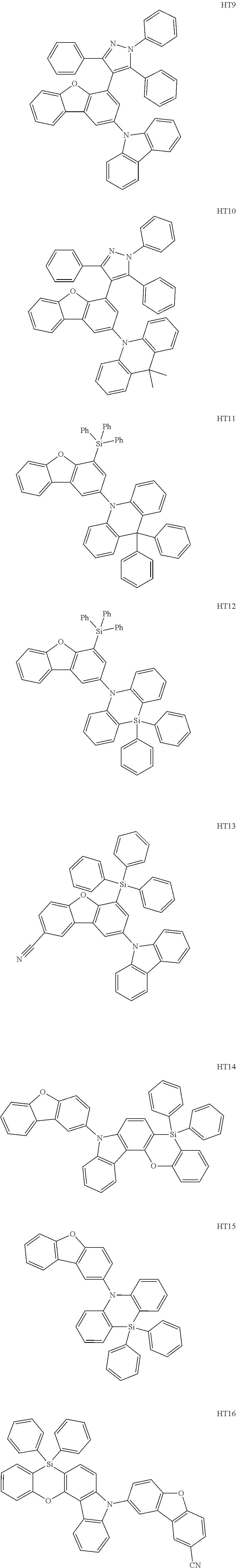

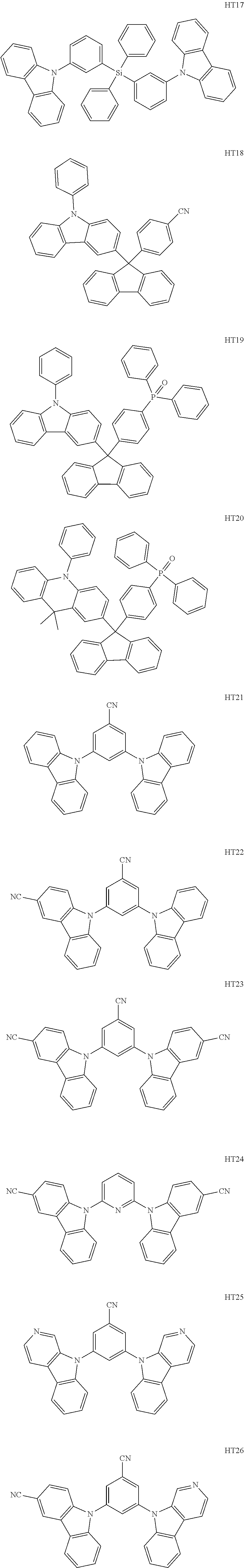

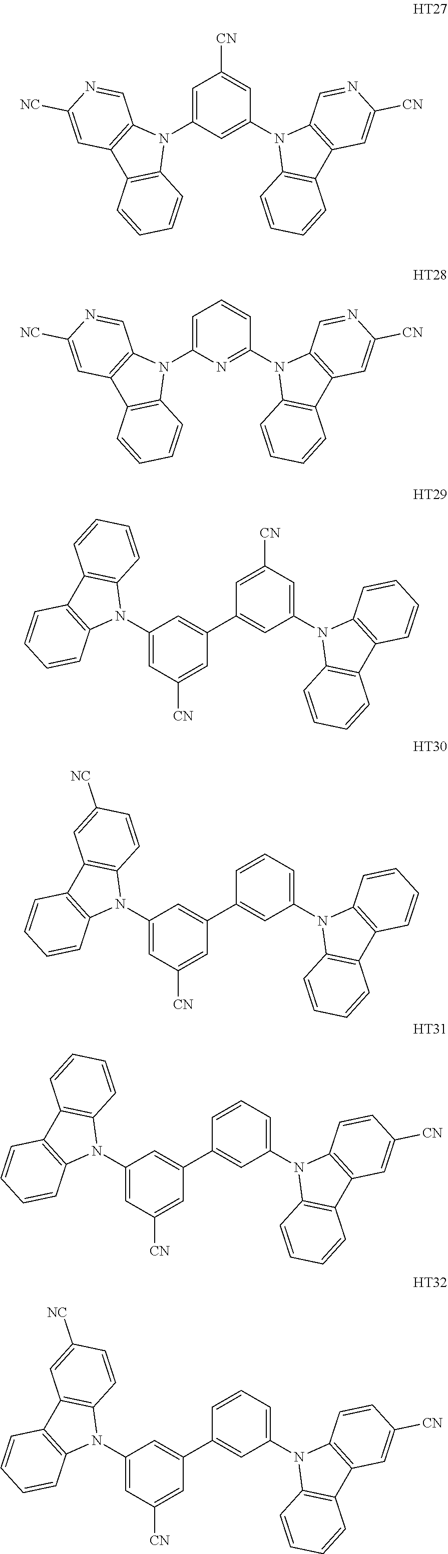 [SMILES: N#Cc1cc(-n2c3ccccc3c3cc(C#N)ncc32)cc(-n2c3ccccc3c3cc(C#N)ncc32)c1 ; N#Cc1cc2c3ccccc3n(-c3cccc(-n4c5ccccc5c5cc(C#N)ncc54)n3)c2cn1 ; N#Cc1cc(-c2cc(C#N)cc(-n3c4ccccc4c4ccccc43)c2)cc(-n2c3ccccc3c3ccccc32)c1 ; N#Cc1cc(-c2cccc(-n3c4ccccc4c4ccccc43)c2)cc(-n2c3ccccc3c3cc(C#N)ccc32)c1 ; N#Cc1cc(-c2cccc(-n3c4ccccc4c4cc(C#N)ccc43)c2)cc(-n2c3ccccc3c3ccccc32)c1 ; N#Cc1cc(-c2cccc(-n3c4ccccc4c4cc(C#N)ccc43)c2)cc(-n2c3ccccc3c3cc(C#N)ccc32)c1]

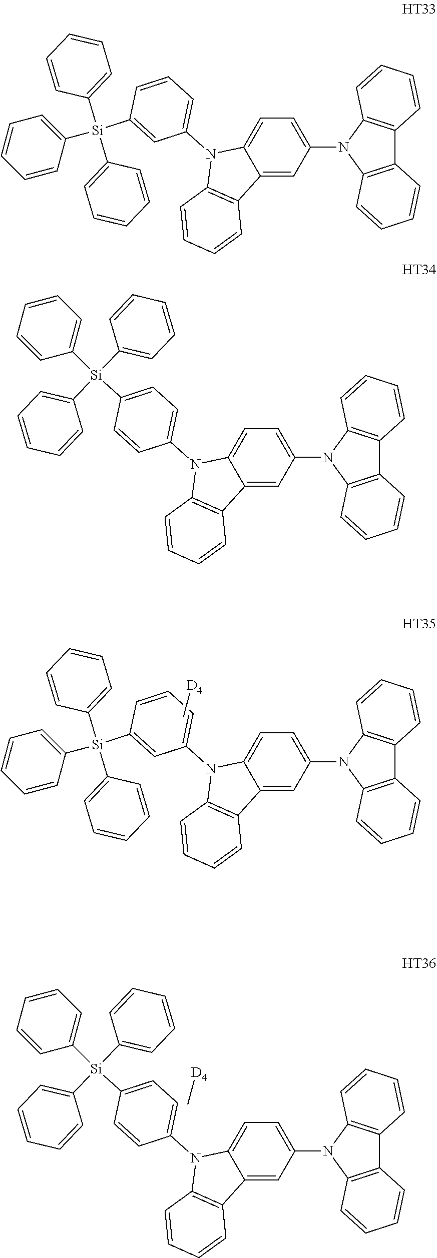

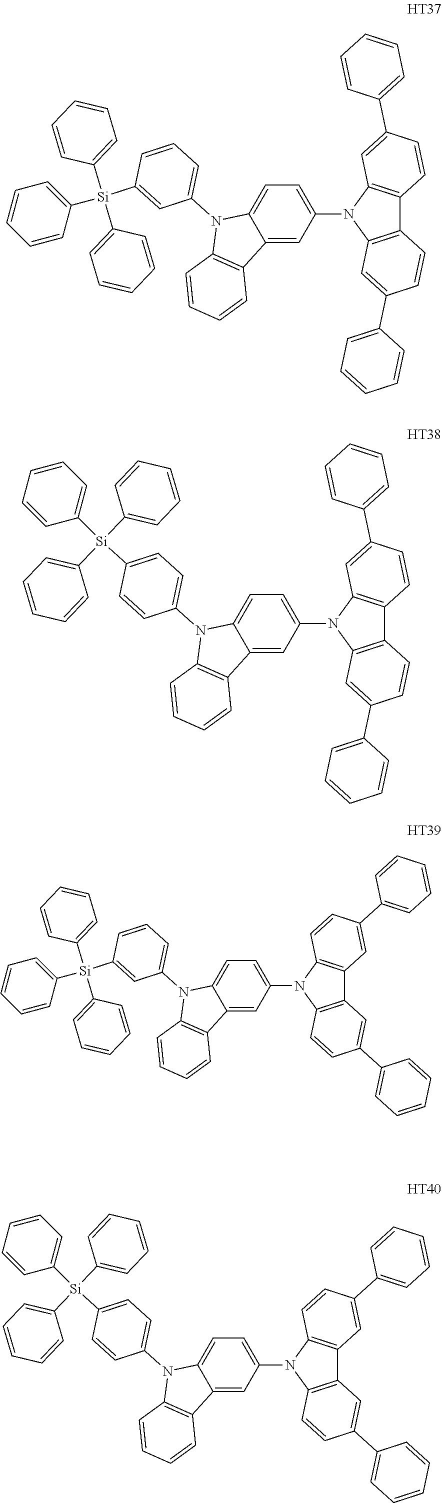 [SMILES: c1ccc(-c2ccc3c4ccc(-c5ccccc5)cc4n(-c4ccc5c(c4)c4ccccc4n5-c4cccc([Si](c5ccccc5)(c5ccccc5)c5ccccc5)c4)c3c2)cc1 ; c1ccc(-c2ccc3c4ccc(-c5ccccc5)cc4n(-c4ccc5c(c4)c4ccccc4n5-c4ccc([Si](c5ccccc5)(c5ccccc5)c5ccccc5)cc4)c3c2)cc1 ; c1ccc(-c2ccc3c(c2)c2cc(-c4ccccc4)ccc2n3-c2ccc3c(c2)c2ccccc2n3-c2cccc([Si](c3ccccc3)(c3ccccc3)c3ccccc3)c2)cc1 ; c1ccc(-c2ccc3c(c2)c2cc(-c4ccccc4)ccc2n3-c2ccc3c(c2)c2ccccc2n3-c2ccc([Si](c3ccccc3)(c3ccccc3)c3ccccc3)cc2)cc1]

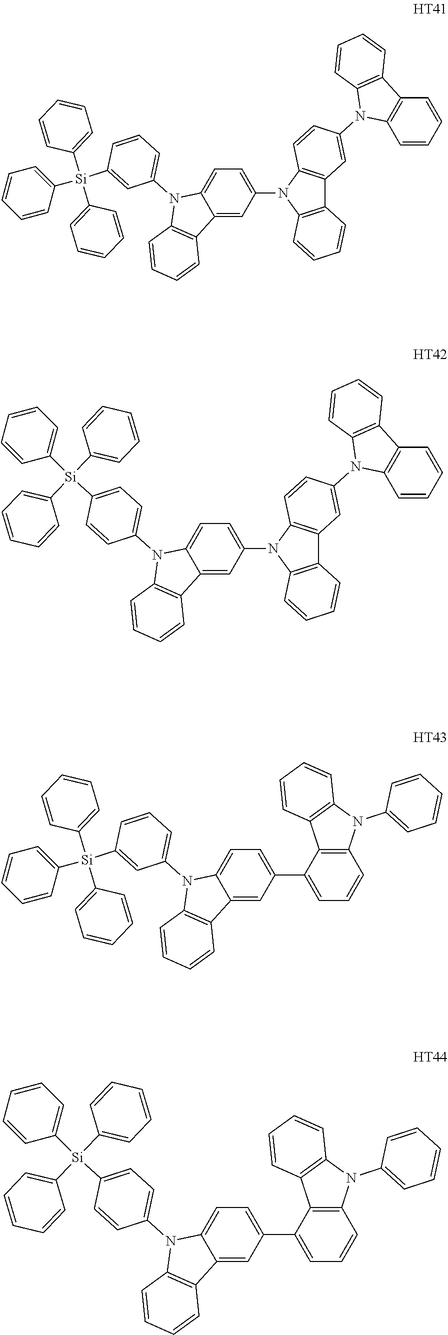 [SMILES: c1ccc([Si](c2ccccc2)(c2ccccc2)c2cccc(-n3c4ccccc4c4cc(-n5c6ccccc6c6cc(-n7c8ccccc8c8ccccc87)ccc65)ccc43)c2)cc1 ; c1ccc([Si](c2ccccc2)(c2ccccc2)c2ccc(-n3c4ccccc4c4cc(-n5c6ccccc6c6cc(-n7c8ccccc8c8ccccc87)ccc65)ccc43)cc2)cc1 ; c1ccc(-n2c3ccccc3c3c(-c4ccc5c(c4)c4ccccc4n5-c4cccc([Si](c5ccccc5)(c5ccccc5)c5ccccc5)c4)cccc32)cc1 ; c1ccc(-n2c3ccccc3c3c(-c4ccc5c(c4)c4ccccc4n5-c4ccc([Si](c5ccccc5)(c5ccccc5)c5ccccc5)cc4)cccc32)cc1]

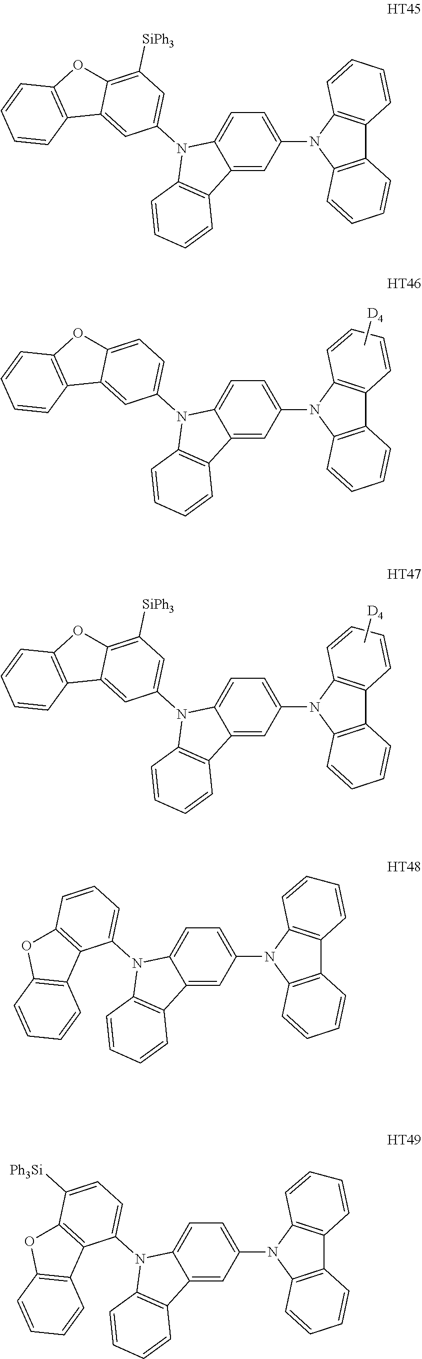

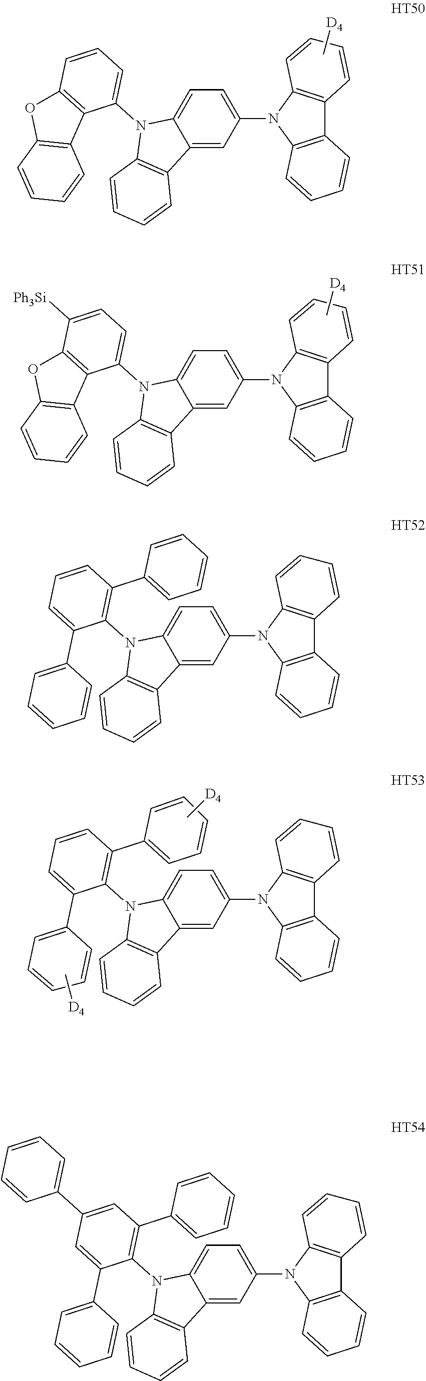

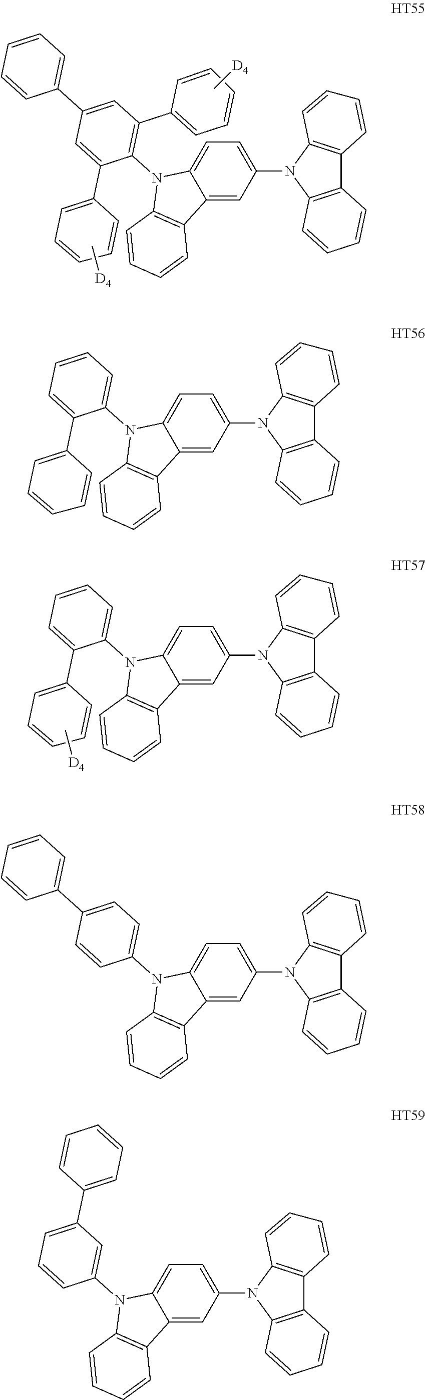

The emission layer EML of an embodiment may further include the third compound represented by Formula M-a below. The third compound may be used a phosphorescent dopant material.

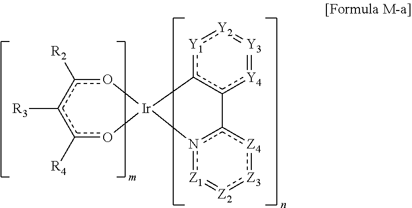

In Formula M-a, Yi to Y₄, and Z₁ to Z₄ may each independently be CR₈₁ or N. R₈₁ to R₈₄ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring.

In Formula M-a, m1 may be 0 or 1, and m2 may be 2 or 3, When m1 is 0, m2 may be 3, and when m1 is 1, m2 may be 2.

The compound represented by Formula M-a may be represented by any one selected from among compounds M-a1 to M-a25 below. However, the compounds M a1 to M-a25 below are presented as an example, and the compound represented by Formula M-a is not limited to those represented by the compounds M-a1 to M-a25 below.

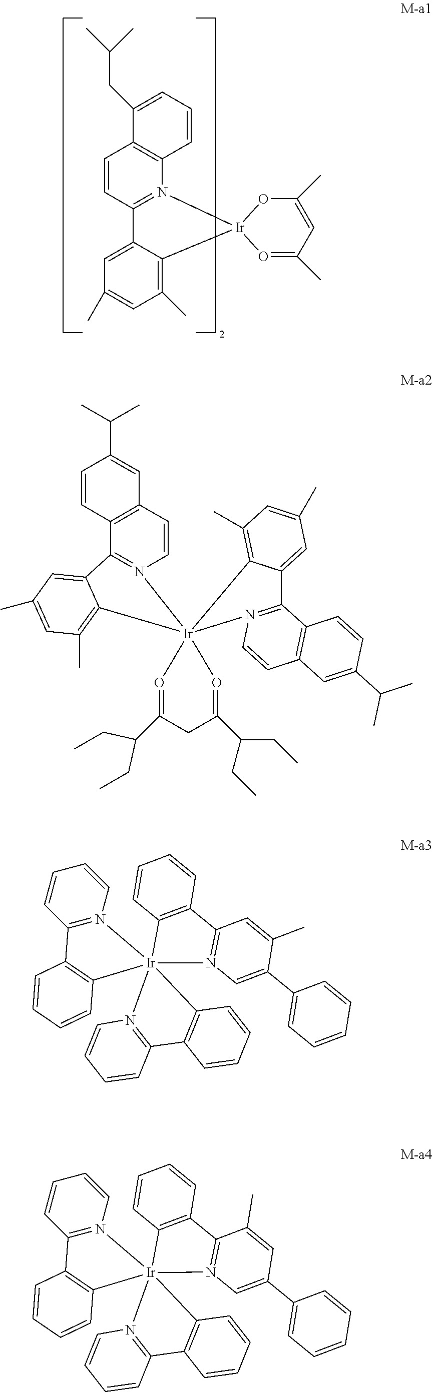

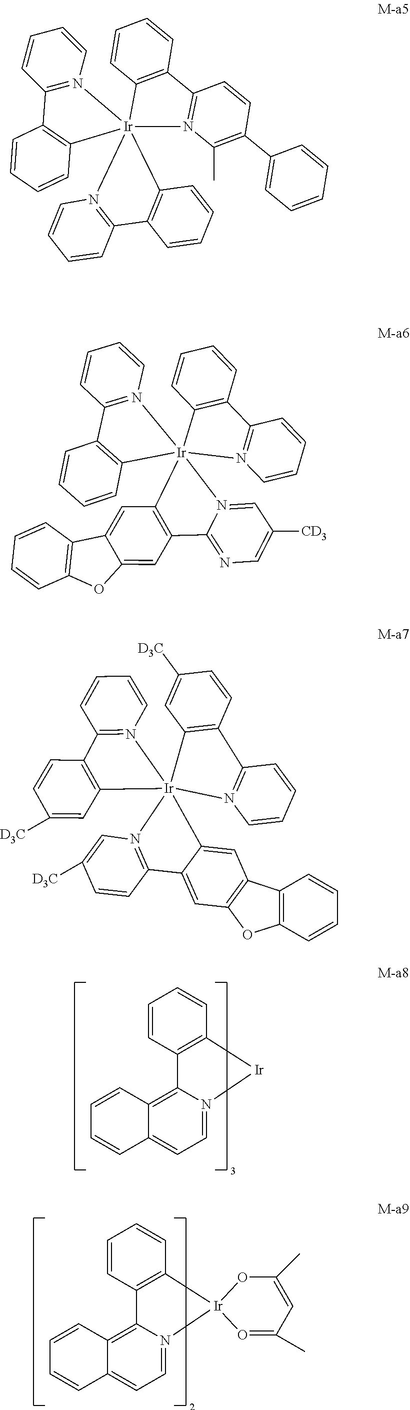

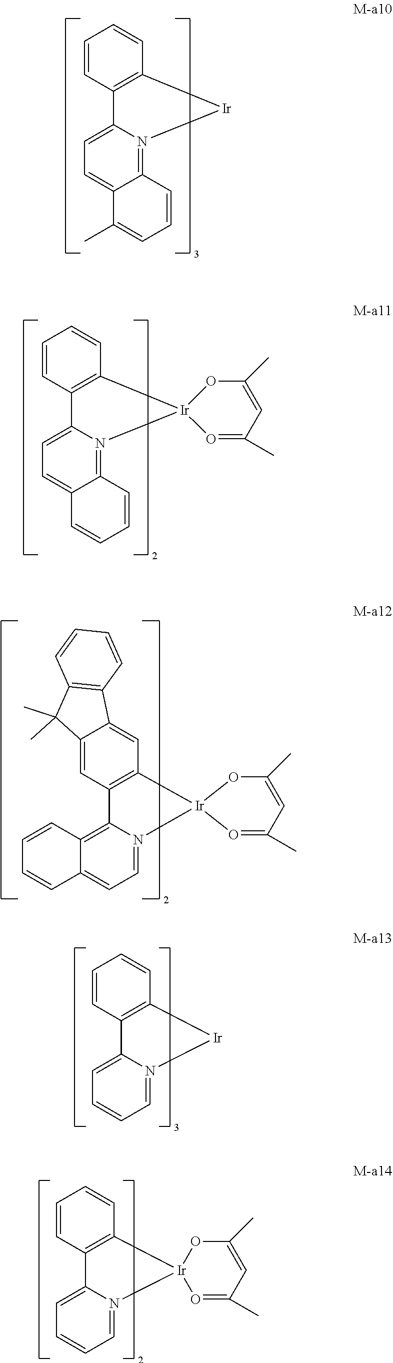

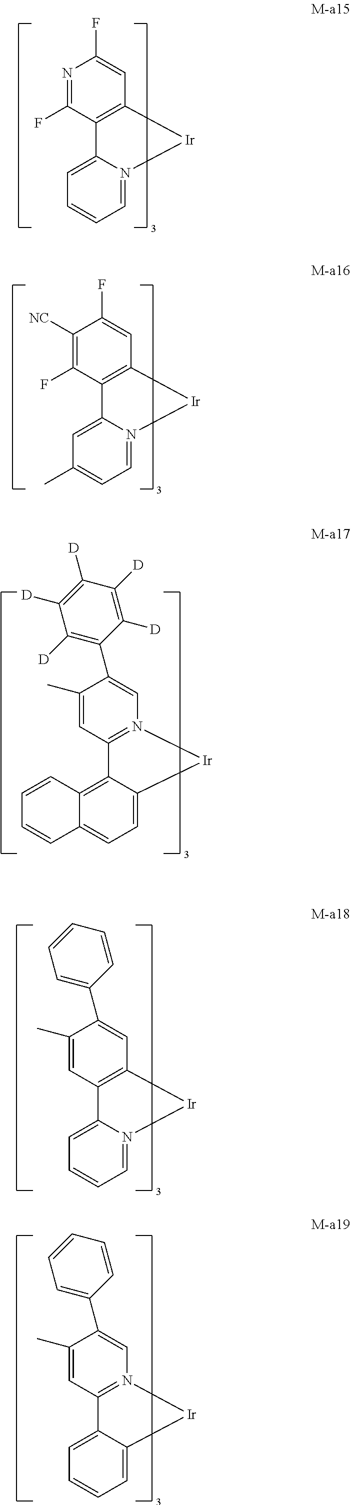

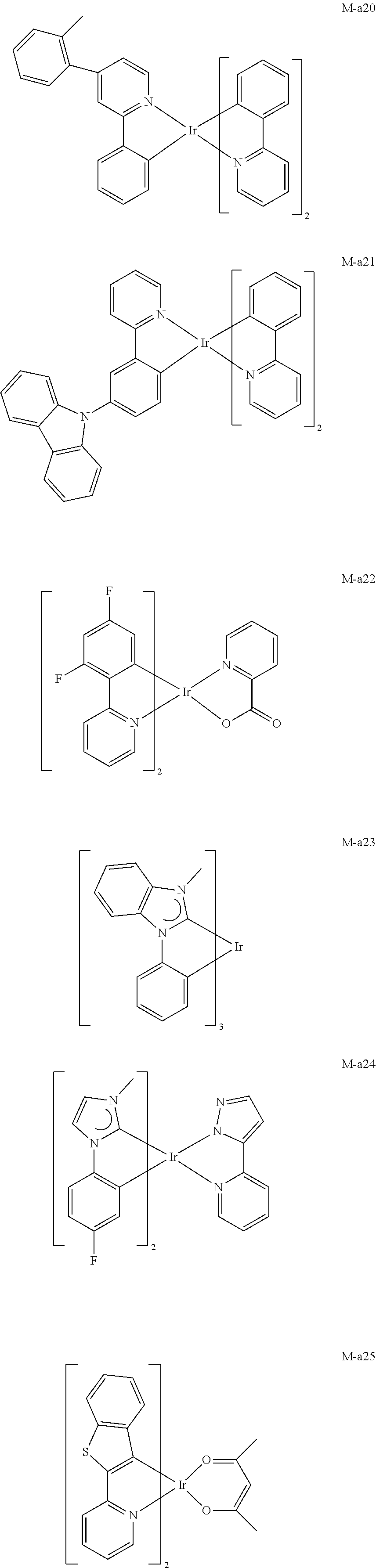

The compounds M-a1 and M-a2 may be used as a red dopant material, and the compounds M-a3 to M-a7 may be used as a green dopant material.

The emission layer EML is provided on the hole transport region HTR. The emission layer EML may have, for example, a thickness of about 100 Å to about 1000 Å or about 100 Å to about 300 Å. The emission layer EML may have a single layer formed of a single material, a single layer formed of a plurality of different materials, or a multilayer structure having a plurality of layers formed of a plurality of different materials.

The emission layer EML may further include compounds which will be further described herein below, in addition to the nitrogen-containing compound, the second compound, and the third compound according to an embodiment.

In the light emitting element ED according to an embodiment, the emission layer EML may include an anthracene derivative, a pyrene derivative, a fluoranthene derivative, a chrysene derivative, a dihydrobenzanthracene derivative, and/or a triphenylene derivative. In one or more embodiments, the emission layer EML may include an anthracene derivative and/or a pyrene derivative.

The emission layer EML may include a compound represented by Formula E-1 below. The compound represented by Formula E-1 below may be used as a fluorescent host material.

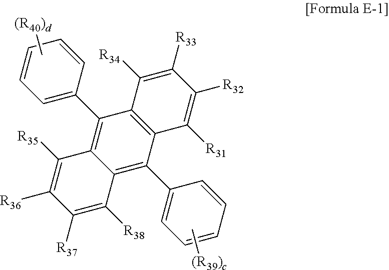

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring. In one or more embodiments, R₃₁ to R₄₀ may be bonded to an adjacent group to form a saturated hydrocarbon ring, an unsaturated hydrocarbon ring, a saturated heterocycle, or an unsaturated heterocycle.

In Formula E-1, c and d may each independently be an integer of 0 to 5. When c is an integer of 2 or greater, a plurality of R₃₉'s may all be the same or at least one may be different from the others. When d is an integer of 2 or greater, a plurality of R₄₀'s may all be the same or at least one may be different from the others. Formula E-1 may be represented by any one selected from among compounds E1 to E19 below.

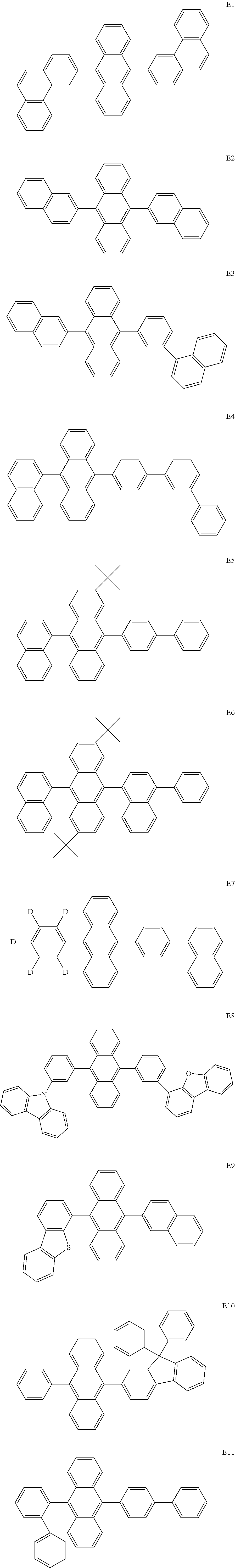

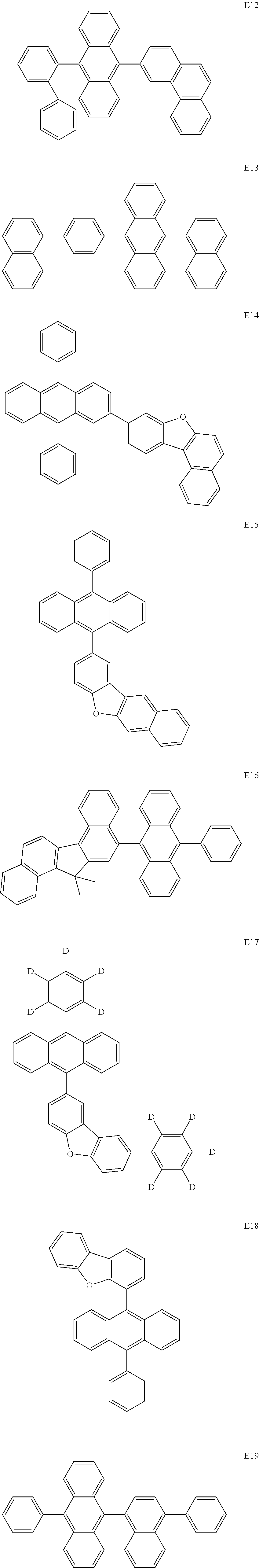

In an embodiment, the emission layer EML may include a compound represented by Formula E-2a and/or Formula E-2b below. The compound represented by Formula E-2a or Formula E-2b may be used as a phosphorescent host material.

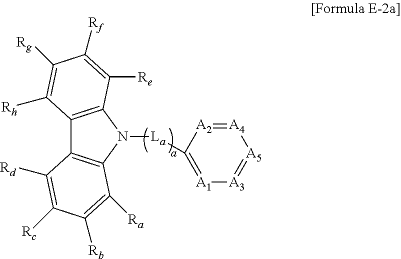

In Formula E-2a, a may be an integer of 0 to 10, and Lₐ may be a direct linkage, a substituted or unsubstituted arylene group having 6 to 30 ring-forming carbon atoms, or a substituted or unsubstituted heteroarylene group having 2 to 30 ring-forming carbon atoms. In one or more embodiments, when a is an integer of 2 or greater, a plurality of Lₐ's may each independently be a substituted or unsubstituted arylene group having 6 to 30 ring-forming carbon atoms, or a substituted or unsubstituted heteroarylene group having 2 to 30 ring-forming carbon atoms.

In addition, in Formula E-2a, A₁ to A₅ may be N or CRᵢ. Rₐ to Rᵢ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring. Rₐ to Rᵢ may be bonded to an adjacent group to form a hydrocarbon ring or a heterocycle containing N, O, S, and the like as a ring-forming atom.

In one or more embodiments, in Formula E-2a, two or three selected from A₁ to A₅ may be N, and the rest may be CRᵢ.

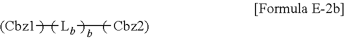

In Formula E-2b, Cbz1 and Cbz2 may each independently be an unsubstituted carbazole group or an aryl-substituted carbazole group having 6 to 30 ring-forming carbon atoms. L_{b} may be a direct linkage, a substituted or unsubstituted arylene group having 6 to 30 ring-forming carbon atoms, or a substituted or unsubstituted heteroarylene group having 2 to 30 ring-forming carbon atoms, b may be an integer of 0 to 10, and when b is an integer of 2 or greater, a plurality of L_{b}'s may each independently be a substituted or unsubstituted arylene group having 6 to 30 ring-forming carbon atoms, or a substituted or unsubstituted heteroarylene group having 2 to 30 ring-forming carbon atoms.

The compound represented by Formula E-2a or Formula E-2b may be represented by any one selected from among compounds from Compound Group E-2 below. However, the compounds listed in Compound Group E-2 below are presented as an example, and the compound represented by Formula E-2a or Formula E-2b is not limited to those listed in Compound Group E-2 below.

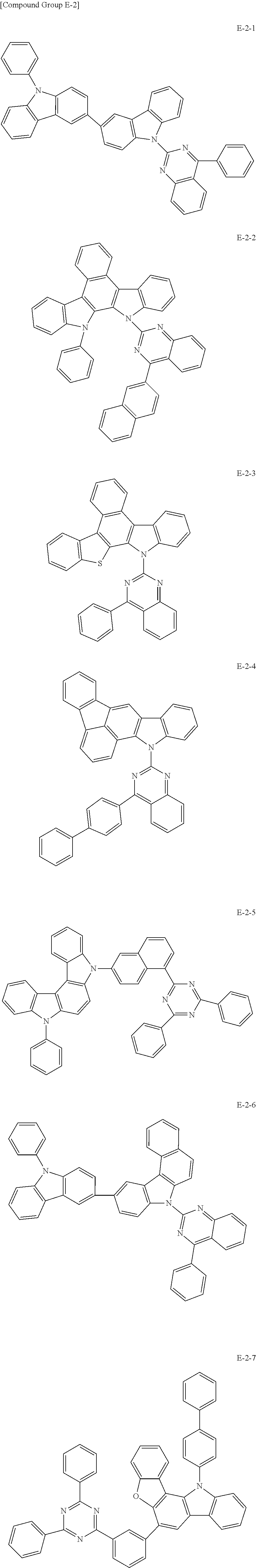

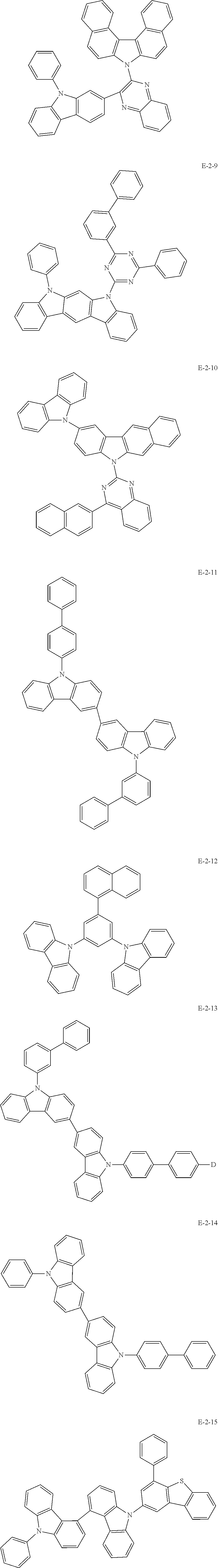

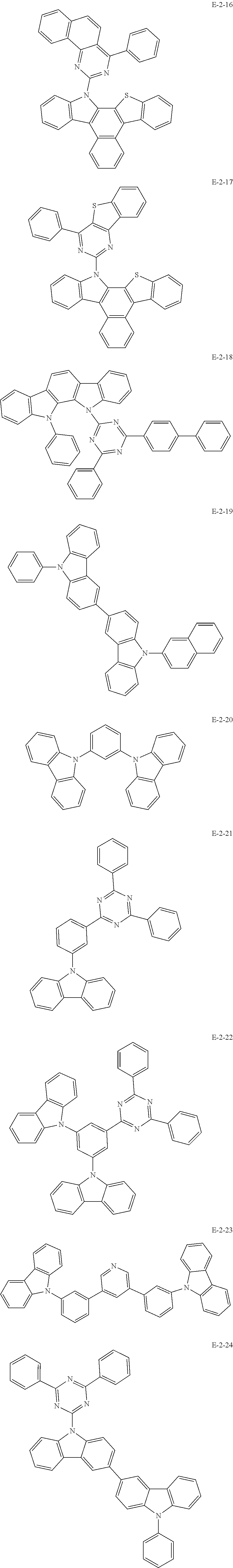

The emission layer EML may further include any suitable material generally used in the art as a host material. For example, the emission layer EML may include, as a host material, at least one among bis(4-(9H-carbazol-9-yl)phenyl)diphenylsilane (BCPDS), (4-(1-(4-(diphenylamino)phenyl)cyclohexyl)phenyl)diphenyl-phosphine oxide (POPCPA), bis[2-(diphenylphosphino)phenyl]ether oxide (DPEPO), 4,4′-bis(N-carbazolyl)-1,1′-biphenyl (CBP), 1,3-bis(carbazolyl-9-yl)benzene (mCP), 2,8-bis(diphenylphosphoryl)dibenzofuran (PPF), 4,4′,4″-tris(carbazol-9-yl)-triphenylamine (TCTA), and 1,3,5-tris(1-phenyl-1H-benzo[d]imidazol-2-yl)benzene (TPBi). However, the embodiment of the present disclosure is not limited thereto, and for example, tris(8-hydroxyquinolino)aluminum (Alq₃), 9,10-di(naphthalene-2-yl)anthracene (ADN), 3-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetrasiloxane (DPSiO₄), and/or the like may be used as a host material.

The emission layer EML may include a compound represented by Formula M-b below. The compound represented by Formula M-b below may be used as a phosphorescent dopant material.

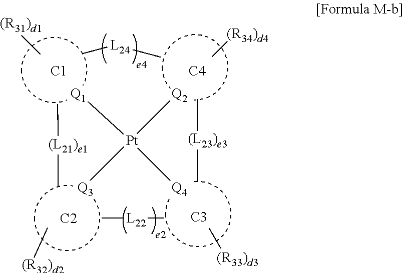

In Formula M-b, Qi to Q₄ may each independently be C or N, and C1 to C4 may each independently be a substituted or unsubstituted hydrocarbon ring having 5 to 30 ring-forming carbon atoms, or a substituted or unsubstituted heterocycle having 2 to 30 ring-forming carbon atoms. L₂₁ to L₂₄ may each independently by a direct linkage,

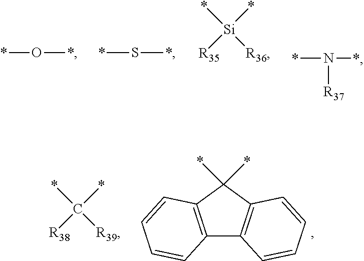

a substituted or unsubstituted divalent alkyl group having 1 to 20 carbon atoms, a substituted or unsubstituted arylene group having 6 to 30 ring-forming carbon atoms, or a substituted or unsubstituted heteroarylene group having 2 to 30 ring-forming carbon atoms, and e1 to e4 may each independently be 0 or 1.

In Formula M-b, R₃₁ to R₃₉ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring. D1 to d4 may each independently be an integer of 0 to 4.

The compound represented by Formula M-b may be used as a blue phosphorescent dopant or a green phosphorescent dopant. The compound represented by Formula M-b may be represented by any one selected from among compounds below. However, the compounds below are presented as an example, and the compound represented by Formula M-b is not limited to those represented by the compounds below.

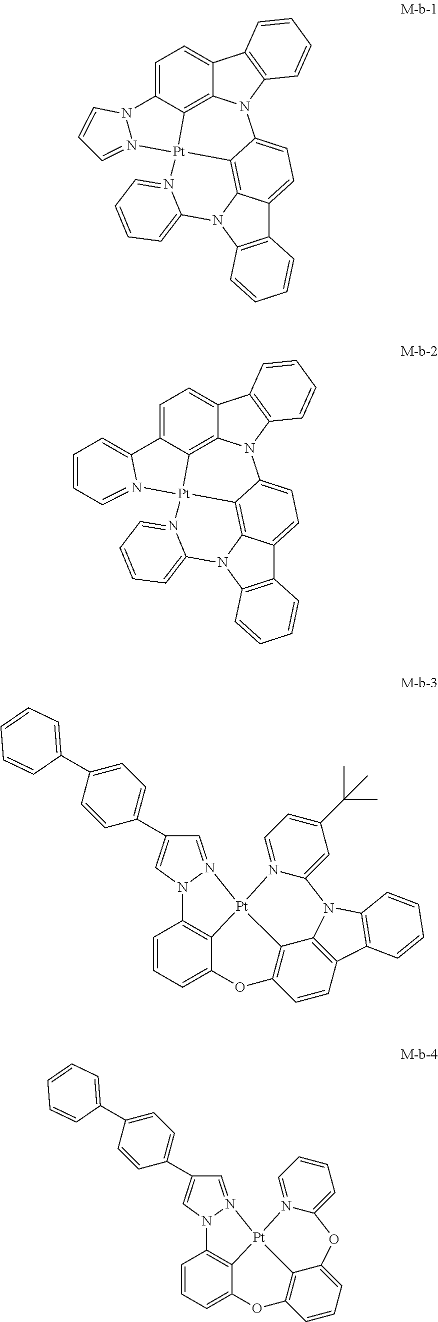

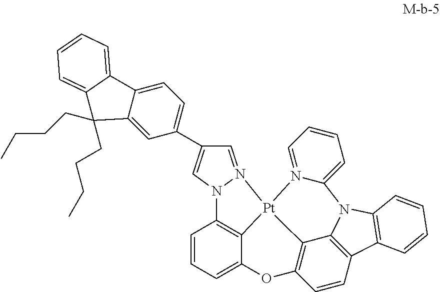

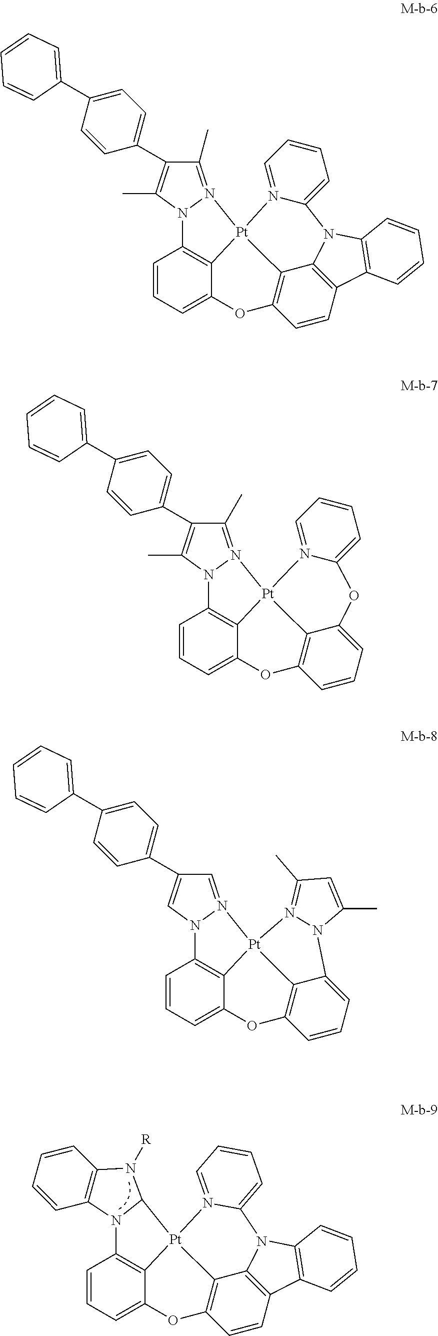

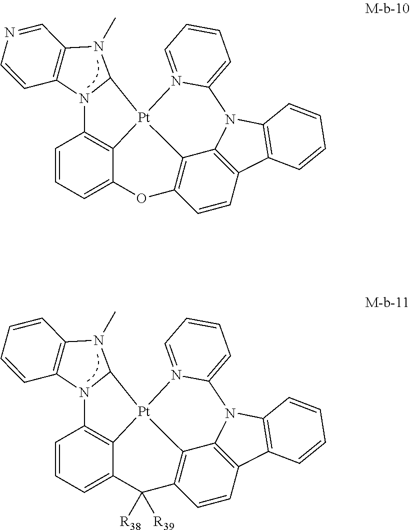

In the compounds above, R, R₃₈, and R₃₉ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

The emission layer EML may further include a compound represented by any one selected from among Formulas F-a to F-c below. The compounds represented by Formulas F-a to F-c below may be used as a fluorescence dopant material.

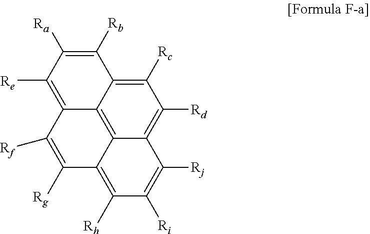

In Formula F-a above, two selected from Rₐ to Rⱼ may each independently be substituted with *—NAr₁Ar₂. The others among Rₐ to Rⱼ which are not substituted with *—NAr₁Ar₂ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In *—NAr₁Ar₂, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. For example, at least one selected from Ar₁ and Ar₂ may be a heteroaryl group containing O or S as a ring-forming atom.

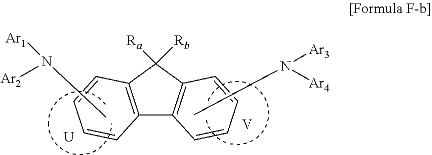

In Formula F-b above, Rₐ and Rb may each independently be a hydrogen atom, a deuterium atom, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring. Ar₁ to Ar₄ may each independently be a substituted or unsubstituted aryl group having 6 to 30 ring-forming carbon atoms, or a substituted or unsubstituted heteroaryl group having 2 to 30 ring-forming carbon atoms.

In Formula F-b, U and V may each independently be a substituted or unsubstituted hydrocarbon ring having 5 to 30 ring-forming carbon atoms, or a substituted or unsubstituted heterocycle having 2 to 30 ring-forming carbon atoms.

In Formula F-b, the number of rings represented by U and V may each independently be 0 or 1. For example, In Formula F-b, when the number of U or V is 1, one ring forms a fused ring in a portion indicated by U or V, and when the number of U or V is 0, it means that no ring indicated by U or V is present. In one or more embodiments, when the number of U is 0 and the number of V is 1, or when the number of U is 1 and the number of V is 0, a fused ring having a fluorene core of Formula F-b may be a cyclic compound having four rings. In addition, when both U and V are 0, the fused ring of Formula F-b may be a cyclic compound having three rings. In addition, when both U and V are 1, the fused ring having a fluorene core of Formula F-b may be a cyclic compound having five rings.

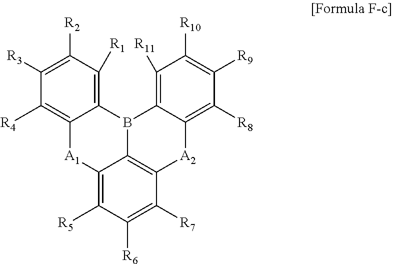

In Formula F-c, A₁ and A₂ may each independently be O, S, Se, or NRₘ, and Rₘ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. R₁ to R₁₁ are each independently a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring.

In Formula F-c, A₁ and A₂ may each independently be bonded to substituents of neighboring rings to form a fused ring. For example, when A₁ and A₂ are each independently NRₘ, A₁ may be bonded to R₄ or R₅ to form a ring. In addition, A₂ may be bonded to R₇ or R₈ to form a ring.

The emission layer EML may include any suitable dopant material generally used in the art. In one or more embodiments, the emission layer EML may include styryl derivatives (e.g., 1,4-bis[2-(3-N-ethylcarbazoryl)vinyl]benzene (BCzVB), 4-(di-p-tolylamino)-4′-[(di-p-tolylamino)styryl]stilbene (DPAVB), and/or N-(4-((E)-2-(6-((E)-4-(diphenylamino)styryl)naphthalen-2-yl)vinyl)phenyl)-N-phenylbenzenamine (N-BDAVBi), 4,4′-bis[2-(4-(N,N-diphenylamino)phenyl)vinyl]biphenyl (DPAVBi), perylene and derivatives thereof (e.g., 2,5,8,11-tetra-t-butylperylene (TBP)), pyrene and/or derivatives thereof (e.g., 1,1′-dipyrene, 1,4-dipyrenylbenzene, 1,4-bis(N,N-diphenylamino)pyrene), and/or the like.

The emission layer EML may further include any suitable phosphorescent dopant material generally used in the art. For example, as a phosphorescent dopant, a metal complex including iridium (Ir), platinum (Pt), osmium (Os), gold (Au), titanium (Ti), zirconium (Zr), hafnium (Hf), europium (Eu), and terbium (Tb), or thulium (Tm) may be used. In one or more embodiments, iridium(III) bis(4,6-difluorophenylpyridinato-N,C2′)picolinate (Flrpic), bis(2,4-difluorophenylpyridinato)-tetrakis(1-pyrazolyl)borate iridium(III) (FIr6), platinum octaethyl porphyrin (PtOEP), and/or the like may be used as a phosphorescent dopant. However, the embodiment of the present disclosure is not limited thereto.

The emission layer EML may include a quantum dot material. The core of a quantum dot may be selected from a Group II-VI compound, a Group III-VI compound, a Group I-III-VI compound, a Group III-V compound, a Group III-II-V compound, a Group IV-VI compound, a Group IV element, a Group IV compound, and a combination thereof.

The Group II-VI compound may be selected from the group consisting of a binary compound selected from the group consisting of CdSe, CdTe, CdS, ZnS, ZnSe, ZnTe, ZnO, HgS, HgSe, HgTe, MgSe, MgS, and a mixture thereof, a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and a mixture thereof, and/or a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, and a mixture thereof.

The Group III-VI compound may include a binary compound such as In₂S₃ and/or In₂Se₃, a ternary compound such as InGaS₃ and/or InGaSe₃, or any combination thereof.

The Group I-III-VI compound may include a ternary compound selected from the group consisting of AgInS, AgInS₂, CuInS, CuInS₂, AgGaS₂, CuGaS₂ CuGaO₂, AgGaO₂, AgAlO₂, and any mixture thereof, and/or a quaternary compound such as AgInGaS₂ and/or CuInGaS₂.

The Group III-V compound may be selected from the group consisting of a binary compound selected from the group consisting of GaN, GaP, GaAs, GaSb, AlN, AlP, AlAs, AlSb, InN, InP, InAs, InSb, and a mixture thereof, a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and a mixture thereof, and a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and a mixture thereof. In one or more embodiments, the Group III-V compound may further include a Group II metal. For example, InZnP, and/or the like may be selected as a Group III-II V compound.

The Group IV-VI compound may be selected from the group consisting of a binary compound selected from the group consisting of SnS, SnSe, SnTe, PbS, PbSe, PbTe, and a mixture thereof, a ternary compound selected from the group consisting of SnSeS, SnSeTe, SnSTe, PbSeS, PbSeTe, PbSTe, SnPbS, SnPbSe, SnPbTe, and a mixture thereof, and a quaternary compound selected from the group consisting of SnPbSSe, SnPbSeTe, SnPbSTe, and a mixture thereof. The Group IV element may be selected from the group consisting of Si, Ge, and a mixture thereof. The Group IV compound may be a binary compound selected from the group consisting of SiC, SiGe, and a mixture thereof.

In this case, the binary compound, the ternary compound, and/or the quaternary compound may be present in particles having a uniform (e.g., substantially uniform) concentration distribution, or may be present in the same particles having a partially different concentration distribution. In addition, a core/shell structure in which one quantum dot surrounds another quantum dot may be present. The core/shell structure may have a concentration gradient in which the concentration of an element present in the shell becomes lower along a direction towards the core.

In some embodiments, a quantum dot may have the core/shell structure including a core having nano-crystals, and a shell surrounding the core, which are described above. The shell of the quantum dot may serve as a protection layer to prevent or reduce chemical deformation of the core so as to keep semiconductor properties, and/or a charging layer to impart electrophoresis properties to the quantum dot. The shell may be a single layer or a plurality of layers. Examples of the shell of the quantum dot may be a metal and/or non-metal oxide, a semiconductor compound, or a combination thereof.

For example, the metal and/or non-metal oxide may be a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄, NiO, and/or a ternary compound such as MgAl₂O₄, CoFe₂O₄, NiFe₂O₄, and/or CoMn₂O₄, but the embodiment of the present disclosure is not limited thereto.

In addition, the semiconductor compound may be, for example, CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but the embodiment of the present disclosure is not limited thereto.

The quantum dot may have, in an emission wavelength spectrum, a full width of half maximum (FWHM) of about 45 nm or less, about 40 nm or less, or about 30 nm or less, and in this range, the color purity and/or the color reproducibility may be improved. In addition, light emitted through the quantum dot is emitted in all (e.g., substantially all) directions, and thus a wide viewing angle may be improved.

In addition, the form of a quantum dot is not particularly limited as long as it is a form generally used in the art, for example, a quantum dot in the form of spherical, pyramidal, multi-arm, and/or cubic nanoparticles, nanotubes, nanowires, nanofibers, nanoplatelets, and/or the like may be used.

The quantum dot may control the colors of emitted light according to the particle size thereof, and thus the quantum dot may have various suitable light emitting colors such as blue, red, green, and/or the like.

In the light emitting element ED according to an embodiment shown in FIGS. 3-6, an electron transport region ETR is provided on the emission layer EML. The electron transport region ETR may include at least one selected from among a hole blocking layer HBL, an electron transport layer ETL, and an electron injection layer EIL, but the embodiment of the present disclosure is not limited thereto.

The electron transport region ETR may have a single layer formed of a single material, a single layer formed of a plurality of different materials, or a multilayer structure having a plurality of layers formed of a plurality of different materials.

For example, the electron transport region ETR may have a single layer structure of an electron injection layer EIL or an electron transport layer ETL, and may have a single layer structure formed of an electron injection material and an electron transport material. In addition, the electron transport region ETR may have a single layer structure formed of a plurality of different materials, or may have a structure in which an electron transport layer ETL/electron injection layer EIL, a hole blocking layer HBL/electron transport layer ETL/electron injection layer EIL, or an electron transport layer ETL/buffer layer/electron injection layer EIL are stacked in order from the emission layer EML, but is not limited thereto. The electron transport region ETR may have a thickness of, for example, about 1000 Å to about 1500 Å.

The electron transport region ETR may be formed using various suitable methods such as a vacuum deposition method, a spin coating method, a cast method, a Langmuir-Blodgett (LB) method, an inkjet printing method, a laser printing method, and/or a laser induced thermal imaging (LITI) method.

The electron transport region ETR may include a compound represented by Formula ET-1 below.

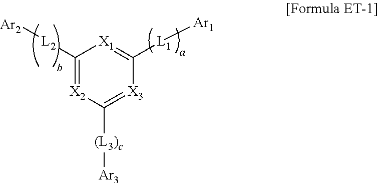

In Formula ET-1, at least one selected from X₁ to X₃ is N and the rest are Crₐ. Rₐ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. Ar₁ to Ar₃ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula ET-1, a to c may each independently be an integer of 0 to 10. In Formula ET-1, L₁ to L₃ may each independently be a direct linkage, a substituted or unsubstituted arylene group having 6 to 30 ring-forming carbon atoms, or a substituted or unsubstituted heteroarylene group having 2 to 30 ring-forming carbon atoms. In one or more embodiments, when a to c are an integer of 2 or greater, L₁ to L₃ may each independently be a substituted or unsubstituted arylene group having 6 to 30 ring-forming carbon atoms, or a substituted or unsubstituted heteroarylene group having 2 to 30 ring-forming carbon atoms.

The electron transport region ETR may include an anthracene-based compound. However, the embodiment of the present disclosure is not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazolyl-1-ylphenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), berylliumbis(benzoquinolin-10-olate (Bebq₂), 9,10-di(naphthalene-2-yl)anthracene (ADN), 1,3-bis[3,5-di(pyridin-3-yl)phenyl]benzene (BmPyPhB), diphenyl(4-(triphenylsilyl)phenyl)phosphine oxide (TSPO1), or a mixture thereof.

The electron transport region ETR may include at least one selected from compounds ET1 to ET36 below.

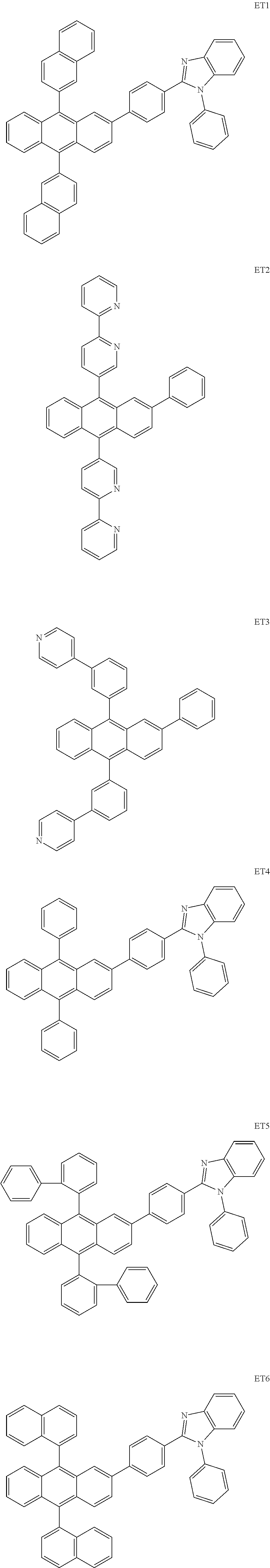

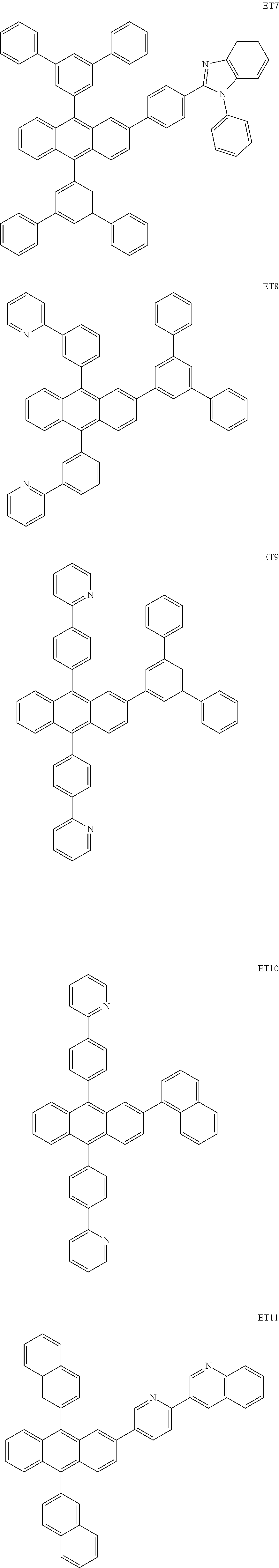 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1]

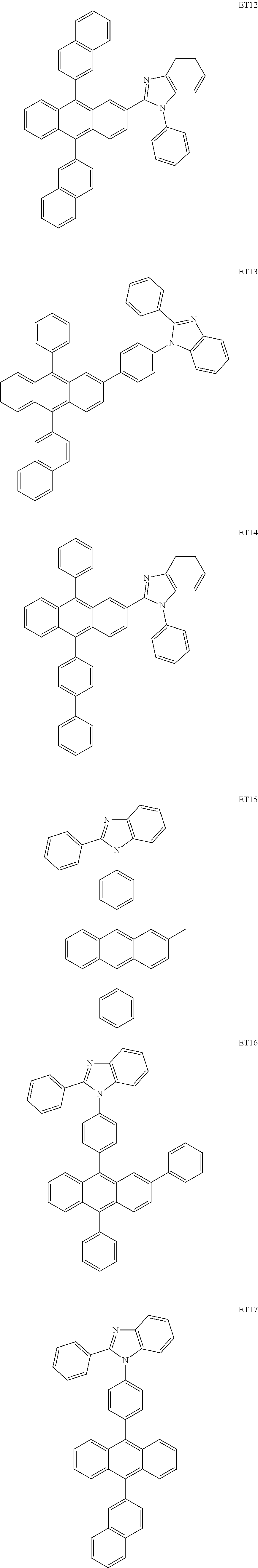

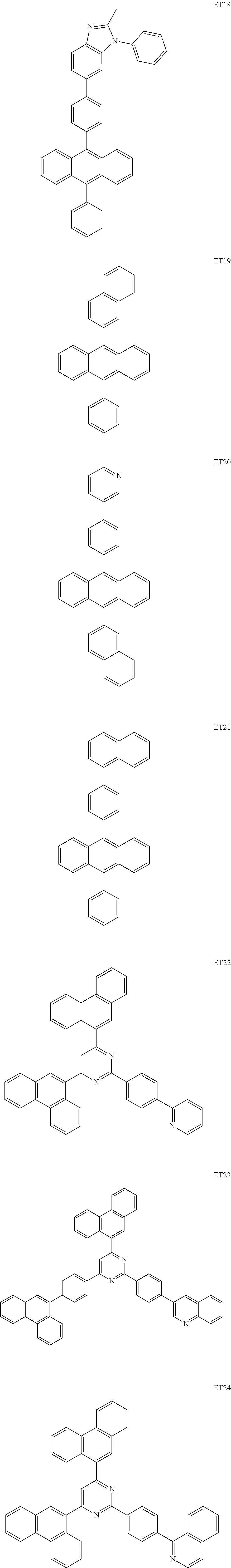

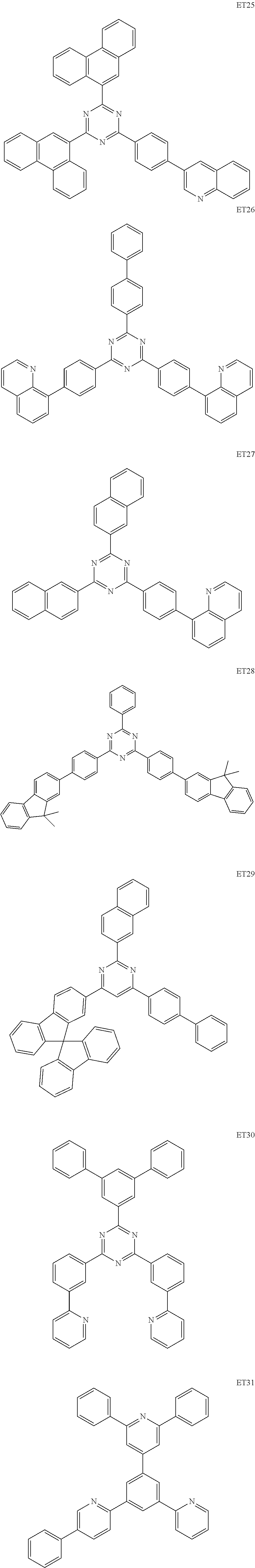

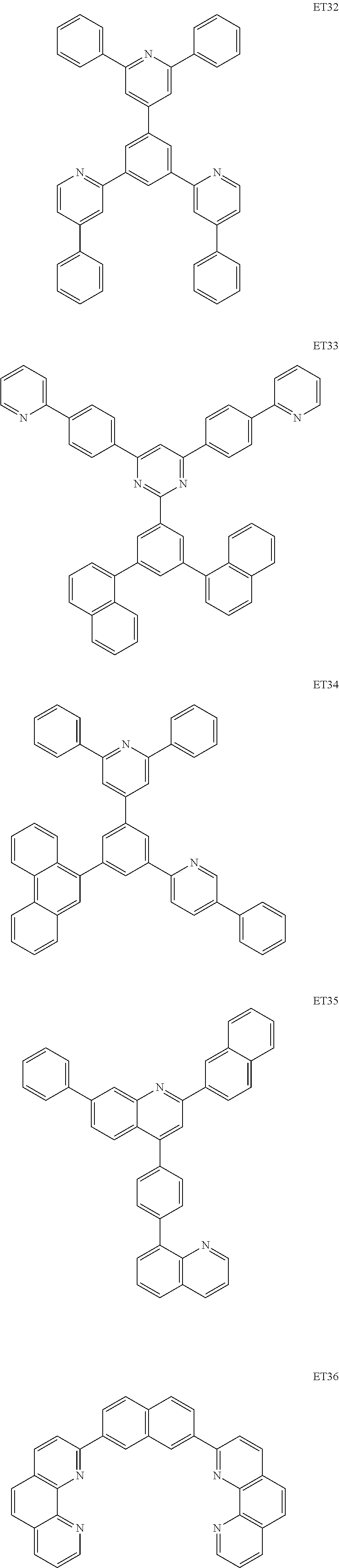 [SMILES: c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In addition, the electron transport region ETR may include halogenated metals such as LiF, NaCl, CsF, RbCl, RbI, CuI, and/or KI, lanthanide metals such as Yb, and/or co-deposition materials of a halogenated metal and/or a lanthanide metal. For example, the electron transport region ETR may include KI:Yb, RbI:Yb, LiF:Yb, and/or the like as a co-deposition material. In one or more embodiments, for the electron transport region ETR, a metal oxide such as Li₂O and/or BaO, and/or 8-hydroxyl-lithium quinolate (Liq), and/or the like may be used, but the embodiment of the present disclosure is limited thereto. The electron transport region ETR may also be formed of a mixture material of an electron transport material and an insulating organo-metal salt. The organo metal salt may be a material having an energy band gap of about 4 eV or greater. In one or more embodiments, the organo-metal salt may include, for example, metal acetates, metal benzoates, metal acetoacetates, metal acetylacetonates, and/or metal stearates.

The electron transport region ETR may further include, for example, at least one selected from 2,9-dimethyl-4,7-diphenyl-1,10-phenanthroline (BCP), diphenyl(4-(triphenylsilyl)phenyl)phosphine oxide (TSPO1), and 4,7-diphenyl-1,10-phenanthroline (Bphen) in addition to the materials described above, but the embodiment of the present disclosure is not limited thereto.

The electron transport region ETR may include the compounds of the electron transport region described above in at least one among the electron injection layer EIL, the electron transport layer ETL, and the hole blocking layer HBL.

When the electron transport region ETR includes the electron transport layer ETL, the electron transport layer ETL may have a thickness of about 100 Å to about 1000 Å, for example, about 150 Å to about 500 Å. When the thickness of the electron transport layer ETL satisfies the above-described range, suitable or satisfactory electron transport properties may be obtained without a substantial increase in driving voltage. When the electron transport region ETR includes the electron injection layer EIL, the electron injection layer EIL may have a thickness of about 1 Å to about 100 Å, for example, about 3 Å to about 90 Å. When the thickness of the electron injection layer EIL satisfies the above-described ranges, suitable or satisfactory electron injection properties may be obtained without a substantial increase in driving voltage.

The second electrode EL2 is provided on the electron transport region ETR. The second electrode EL2 may be a common electrode. The second electrode EL2 may be a cathode or an anode but the embodiment of the present disclosure is not limited thereto. For example, when the first electrode EL1 is an anode, the second electrode EL2 may be a cathode, and when the first electrode EL1 is a cathode, the second electrode EL2 may be an anode. The second electrode EL2 may include at least one selected from Ag, Mg, Cu, Al, Pt, Pd, Au, Ni, Nd, Ir, Cr, Li, Ca, LiF, Mo, Ti, W, In, Sn, and Zn, two or more compounds selected therefrom, two or more mixtures selected therefrom, and/or an oxide thereof.

The second electrode EL2 may be a transmissive electrode, a transflective electrode, or a reflective electrode. When the second electrode EL2 is a transmissive electrode, the second electrode EL2 may be formed of a transparent metal oxide, for example, indium tin oxide (ITO), indium zinc oxide (IZO), zinc oxide (ZnO), indium tin zinc oxide (ITZO), and/or the like.

When the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca (a stack structure of LiF and Ca), LiF/Al (a stack structure of LiF and Al), Mo, Ti, W, a compound thereof, and/or a mixture thereof (e.g., AgMg, AgYb, and/or MgYb). In one or more embodiments, the second electrode EL2 may have a multilayer structure including a reflective film or a transflective film formed of the above-described materials, and a transparent conductive film formed of indium tin oxide (ITO), indium zinc oxide (IZO), zinc oxide (ZnO), indium tin zinc oxide (ITZO), and/or the like. For example, the second electrode EL2 may include the above-described metal materials, a combination of two or more metal materials selected from the above-described metal materials, and/or oxides of the above-described metal materials.

In one or more embodiments, the second electrode EL2 may be connected with an auxiliary electrode. When the second electrode EL2 is connected with the auxiliary electrode, the resistance of the second electrode EL2 may decrease.

In one or more embodiments, a capping layer CPL may be further on the second electrode EL2 of the light emitting element ED according to an embodiment. The capping layer CPL may include a multilayer or a single layer.

In an embodiment, the capping layer CPL may be an organic layer or an inorganic layer. For example, when the capping layer CPL includes an inorganic material, the inorganic material may include an alkali metal compound such as LiF, an alkaline earth metal compound such as MgF₂, SiON, SiNx, SiOy, and/or the like.

For example, when the capping layer CPL includes an organic material, the organic material may include α-NPD, NPB, TPD, m-MTDATA, Alq₃ CuPc, N4,N4,N4′,N4′-tetra(biphenyl-4-yl) biphenyl-4,4′-diamine (TPD15), 4,4′,4″-tris(carbazol-9-yl)triphenylamine (TCTA), and/or the like, and/or may include epoxy resins or acrylates such as methacrylates. However, the embodiment of the present disclosure is not limited thereto, and the capping layer CPL may include compounds P1 to P5 below.

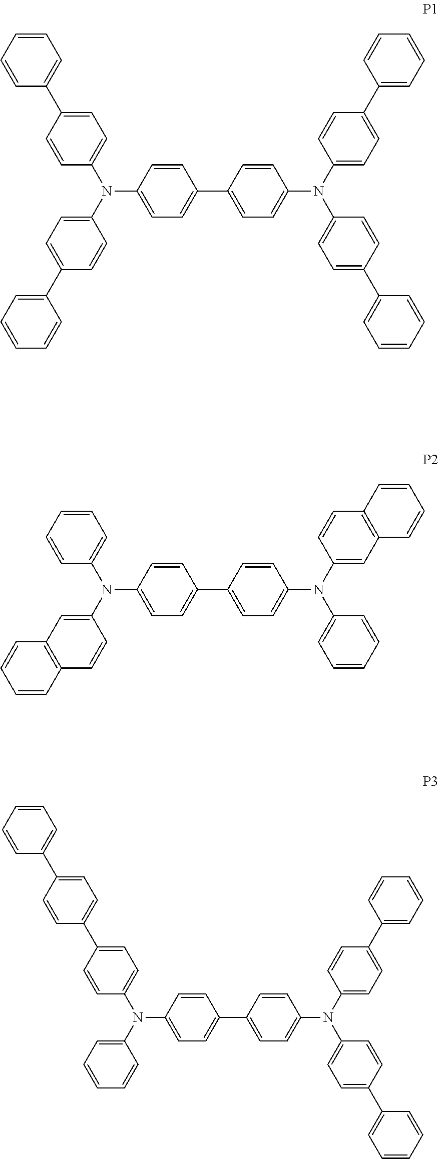 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

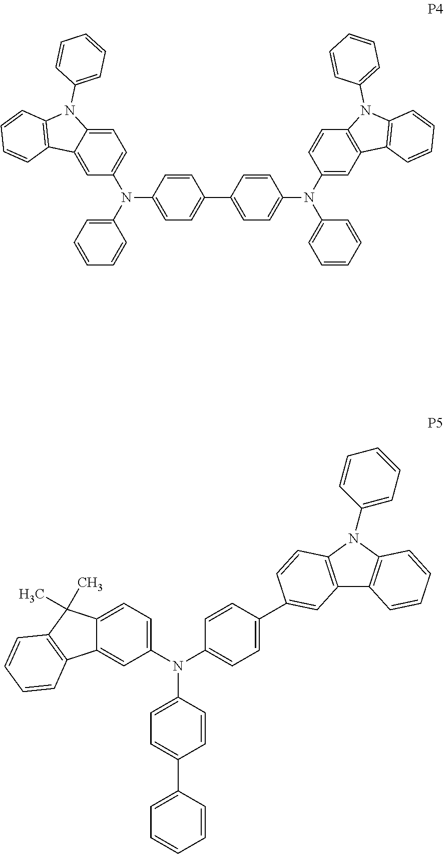 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

In one or more embodiments, the capping layer CPL may have a refractive index of about 1.6 or greater. For example, the capping layer CPL may have a refractive index of about 1.6 or greater in a wavelength range of about 550 nm to about 660 nm.

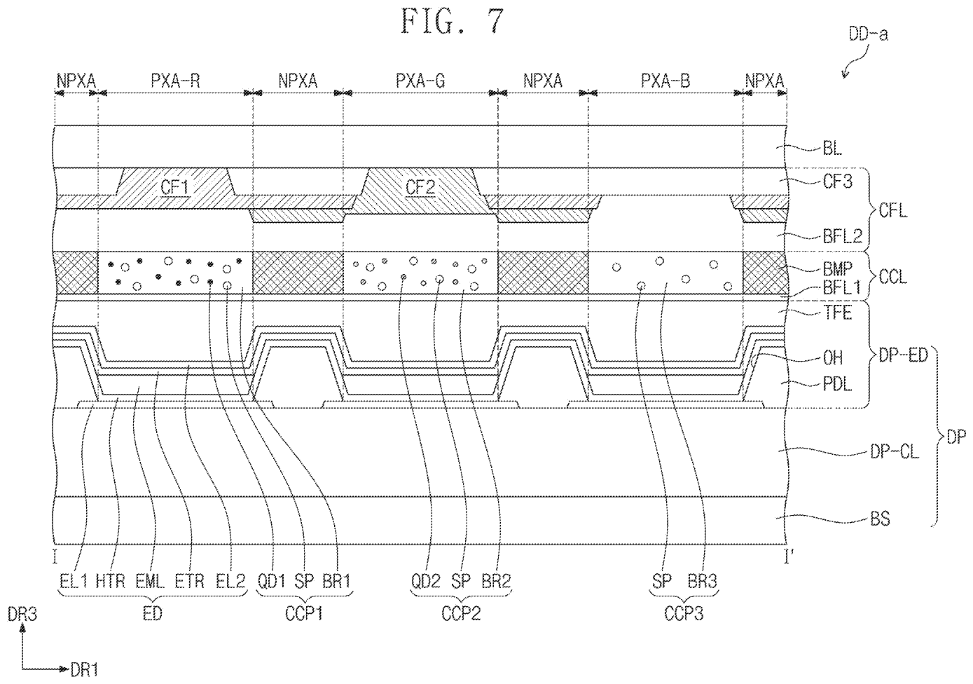

FIGS. 7-10 are each a cross-sectional view of a display device according to an embodiment. Hereinafter, in the description of the display device according to an embodiment with reference to FIGS. 7-10, content overlapping the one described above with reference to FIGS. 1-6 will not be described again here, and the differences will be mainly described.

Referring to FIG. 7, a display device DD-a according to an embodiment may include a display panel DP having a display element layer DP-ED, a light control layer CCL on the display panel DP, and a color filter layer CFL.

In an embodiment shown in FIG. 7, the display panel DP may include a base layer BS, a circuit layer DP-CL provided on the base layer BS, and a display element layer DP-ED, and the element layer DP-ED may include a light emitting element ED.

The light emitting element ED may include a first electrode EL1, a hole transport region HTR on the first electrode EL1, an emission layer EML on the hole transport region HTR, an electron transport region ETR on the emission layer EML, and a second electrode EL2 on the electron transport region ETR. A structure of the light emitting element ED shown in FIG. 7 may be the same as the structure of the light emitting element of FIGS. 3-6 described above.

The emission layer EML of the light emitting element ED included in a display device DD-a according to an embodiment may include the nitrogen-containing compound according to an embodiment described above.

Referring to FIG. 7, the emission layer EML may be in the openings OH defined in the pixel defining films PDL. For example, the emission layer EML separated by the pixel defining films PDL and provided corresponding to each of light emitting regions PXA-R, PXA-G, and PXA-B may emit light in the same wavelength ranges. In the display device DD-a of an embodiment, the emission layer EML may emit blue light. In one or more embodiments, the emission layer EML may be provided as a common layer throughout the light emitting regions PXA-R, PXA-G, and PXA-B.

The light control layer CCL may be on the display panel DP. The light control layer CCL may include a light converter. The light converter may be a quantum dot and/or a phosphor. The light converter may wavelength-convert the provided light and emit the wavelength-converted light. In one or more embodiments, the light control layer CCL may be a layer containing quantum dots and/or phosphors.

The light control layer CCL may include a plurality of light control units CCP1, CCP2, and CCP3. The light control units CCP1, CCP2, and CCP3 may be spaced apart from each other.

Referring to FIG. 7, a division pattern BMP may be between the light control units CCP1, CCP2, and CCP3 spaced apart from each other, but the embodiment of the present disclosure is not limited thereto. In FIG. 7, the division pattern BMP is shown to non-overlap the light control units CCP1, CCP2, and CCP3, but edges of the light control units CCP1, CCP2, and CCP3 may overlap at least a portion of the division pattern BMP.

The light control layer CCL may include a first light control unit CCP1 including a first quantum dot QD1 for converting a first color light provided from the light emitting element ED into a second color light, a second light control unit CCP2 including a second quantum dot QD2 for converting the first color light into a third color light, and a third light control unit CCP3 transmitting the first color light.

In an embodiment, the first light control unit CCP1 may provide red light, which is the second color light, and the second light control unit CCP2 may provide green light, which is the third color light. The third light control unit CCP3 may transmit and provide blue light, which is the first color light provided from the light emitting element ED. For example, the first quantum dot QD1 may be a red quantum dot and the second quantum dot QD2 may be a green quantum dot. The same descriptions above may be applied to the quantum dots QD1 and QD2.

In addition, the light control layer CCL may further include scatterers SP (e.g., light scatterers SP). The first light control unit CCP1 may include the first quantum dot QD1 and the scatterers SP, the second light control unit CCP2 may include the second quantum dot QD2 and the scatterers SP, and the third light control unit CCP3 may not include a quantum dot but may include the scatterers SP.

The scatterers SP may be inorganic particles. For example, the scatterers SP may include at least one selected from among TiO₂, ZnO, Al₂O₃, SiO₂, and hollow silica. The scatterers SP may include any one selected from among TiO₂, ZnO, Al₂O₃, SiO₂, and hollow silica, and/or may be a mixture of two or more materials selected from TiO₂, ZnO, Al₂O₃, SiO₂, and hollow silica.

The first light control unit CCP1, the second light control unit CCP2, and the third light control unit CCP3 may each include base resins BR1, BR2, and BR3 for dispersing the quantum dots QD1 and QD2 and the scatterers SP. In an embodiment, the first light control unit CCP1 may include the first quantum dot QD1 and the scatterers SP dispersed in the first base resin BR1, the second light control unit CCP2 may include the second quantum dot QD2 and the scatterers SP dispersed in the second base resin BR2, and the third light control unit CCP3 may include the scatterers SP dispersed in the third base resin BR3.

The base resins BR1, BR2, and BR3 are a medium in which the quantum dots QD1 and QD2 and the scatterers SP are dispersed, and may be formed of various suitable resin compositions, which may be generally referred to as a binder. For example, the base resins BR1, BR2, and BR3 may be an acrylic resin, a urethane-based resin, a silicone-based resin, an epoxy-based resin, and/or the like. The base resins BR1, BR2, and BR3 may be a transparent resin. In an embodiment, the first base resin BR1, the second base resin BR2, and the third base resin BR3 may each be the same as or different from each other.

The light control layer CCL may include a barrier layer BFL1. The barrier layer BFL1 may serve to prevent or reduce introduction of moisture and/or oxygen (hereinafter referred to as “moisture/oxygen”). The barrier layer BFL1 may be on the light control units CCP1, CCP2, and CCP3 to prevent or reduce exposure of the light control units CCP1, CCP2, and CCP3 to moisture/oxygen. In one or more embodiments, the barrier layer BFL1 may cover the light control units CCP1, CCP2, and CCP3. In addition, a barrier layer BFL2 may be provided between the light control units CCP1, CCP2, and CCP3 and filters CF1, CF2, and CF3.

The barrier layers BFL1 and BFL2 may include at least one inorganic layer. In one or more embodiments, the barrier layers BFL1 and BFL2 may be formed of an inorganic material. For example, the barrier layers BFL1 and BFL2 may be formed including silicon nitride, aluminum nitride, zirconium nitride, titanium nitride, hafnium nitride, tantalum nitride, silicon oxide, aluminum oxide, titanium oxide, tin oxide, cerium oxide, silicon oxynitride, and/or a metal thin film in which light transmittance is secured, and/or the like. In one or more embodiments, the barrier layers BFL1 and BFL2 may further include an organic film. The barrier layers BFL1 and BFL2 may be formed of a single layer or a plurality of layers.

In the display device DD-a of an embodiment, the color filter layer CFL may be on the light control layer CCL. For example, the color filter layer CFL may be directly on the light control layer CCL. In this case, the barrier layer BFL2 may be omitted.

The color filter layer CFL may include filters CF1, CF2, and CF3. In one or more embodiments, the color filter layer CFL may include a first filter CF1 transmitting a second color light, a second filter CF2 transmitting a third color light, and a third filter CF3 transmitting a first color light. For example, the first filter CF1 may be a red filter, the second filter CF2 may be a green filter, and the third filter CF3 may be a blue filter. The filters CF1, CF2, and CF3 may each include a polymer photosensitive resin, a pigment and/or a dye. The first filter CF1 may include a red pigment and/or a red dye, the second filter CF2 may include a green pigment and/or a green dye, and the third filter CF3 may include a blue pigment and/or a blue dye. In one or more embodiments, the embodiment of the present disclosure is not limited thereto, and the third filter CF3 may not include a pigment or a dye. The third filter CF3 may include a polymer photosensitive resin, but not include a pigment or a dye. The third filter CF3 may be transparent. The third filter CF3 may be formed of a transparent photosensitive resin.

In addition, in an embodiment, the first filter CF1 and the second filter CF2 may be yellow filters. The first filter CF1 and the second filter CF2 may not be separated and may be provided as a single body. The first to third filters CF1, CF2, and CF3 may be corresponding to the red light emitting region PXA-R, the green light emitting region PXA-G, and the blue light emitting region PXA-B, respectively.

In one or more embodiments, the color filter layer CFL may include a light blocking unit. The color filter layer CFL may include the light blocking unit disposed to overlap the boundaries of the neighboring filters CF1, CF2, and CF3. The light blocking unit may be a black matrix. The light blocking unit may be formed including an organic light blocking material or an inorganic light blocking material, both including a black pigment and/or a black dye. The light blocking unit may separate boundaries between the adjacent filters CF1, CF2, and CF3. In addition, in an embodiment, the light blocking unit may be formed of a blue filter.

The base substrate BL may be on the color filter layer CFL. The base substrate BL may be a member providing a base surface on which the color filter layer CFL and the light control layer CCL are provided. The base substrate BL may be a glass substrate, a metal substrate, a plastic substrate, and/or the like. However, the embodiment of the present disclosure is not limited thereto, and the base substrate BL may be an inorganic layer, an organic layer, or a composite material layer. In one or more embodiments, the base substrate BL may be omitted.

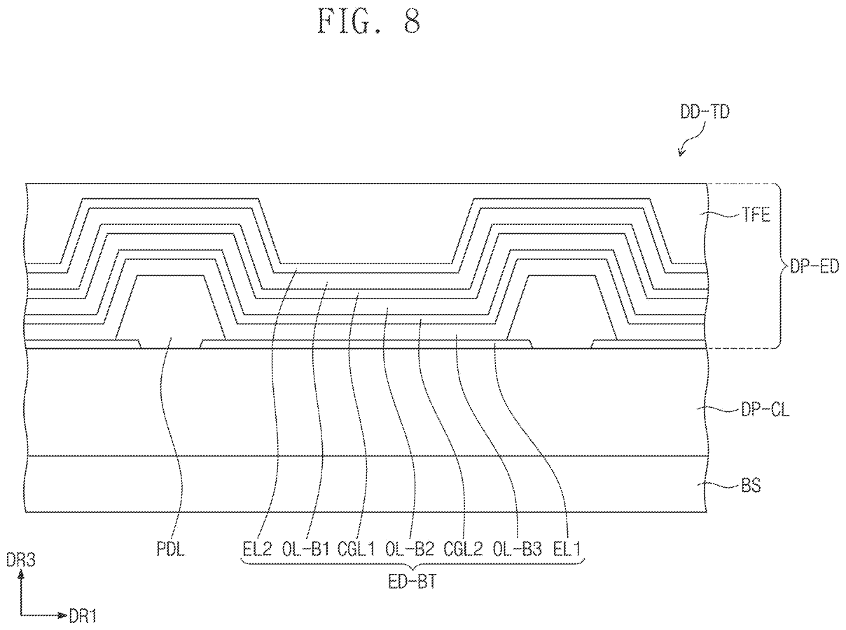

FIG. 8 is a cross-sectional view showing a portion of a display device according to an embodiment; FIG. 8 shows another embodiment of a portion corresponding to the display panel DP of FIG. 7. In a display device DD-TD of an embodiment, a light emitting element ED-BT may include a plurality of light emitting structures OL-B1, OL-B2, and OL-B3. The light emitting element ED-BT may include the first electrode EL1 and the second electrode EL2 facing each other, and the plurality of light emitting structures OL-B1, OL-B2, and OL-B3 provided by being sequentially stacked in a thickness direction between the first electrode EL1 and the second electrode EL2. At least one selected from the light emitting structures OL-B1, OL-B2, and OL-B3 may include the nitrogen-containing compound according to an embodiment described above. The light emitting structures OL-B1, OL-B2, and OL-B3 each may include the emission layer EML (FIG. 7), a hole transport region HTR and an electron transport region ETR provided with the emission layer EML (FIG. 7) therebetween.

In one or more embodiments, the light emitting element ED-BT included in the display device DD-TD of an embodiment may be a light emitting element having a tandem structure including a plurality of emission layers.

In an embodiment shown in FIG. 8, light emitted from each of the light emitting structures OL-B1, OL-B2, and OL-B3 may all be blue light. However, the embodiment of the present disclosure is not limited thereto, and wavelength ranges of light emitted from each of the light emitting structures OL-B1, OL-B2, and OL-B3 may be different from each other. For example, the light emitting element ED-BT including the plurality of light emitting structures OL-B1, OL-B2, and OL-B3 emitting light in different wavelength ranges may emit white light.

Charge generation layers CGL1 and CGL2 may be between neighboring light emitting structures OL-B1, OL-B2, and OL-B3. The charge generation layers CGL1 and CGL2 may include a p-type charge generation layer and/or an n-type charge generation layer.

At least one selected from the light emitting structures OL-B1, OL-B2, and OL-B3 included in the display device DD-TD according to an embodiment may include the nitrogen-containing compound according to an embodiment described above. In one or more embodiments, at least one of the plurality of emission layers included in the light emitting element ED-BT may include a nitrogen-containing compound according to an embodiment.

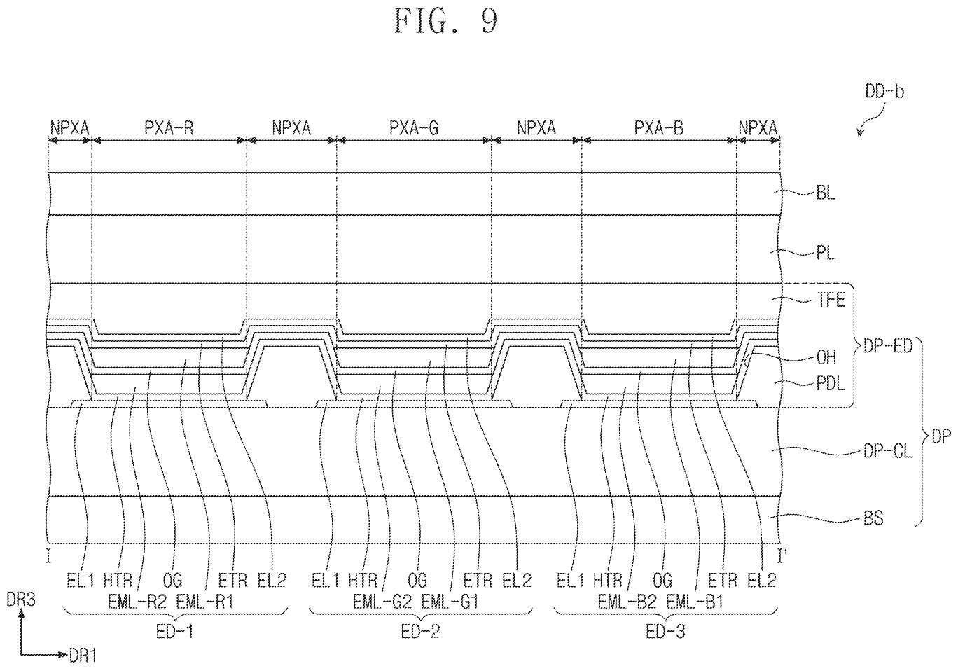

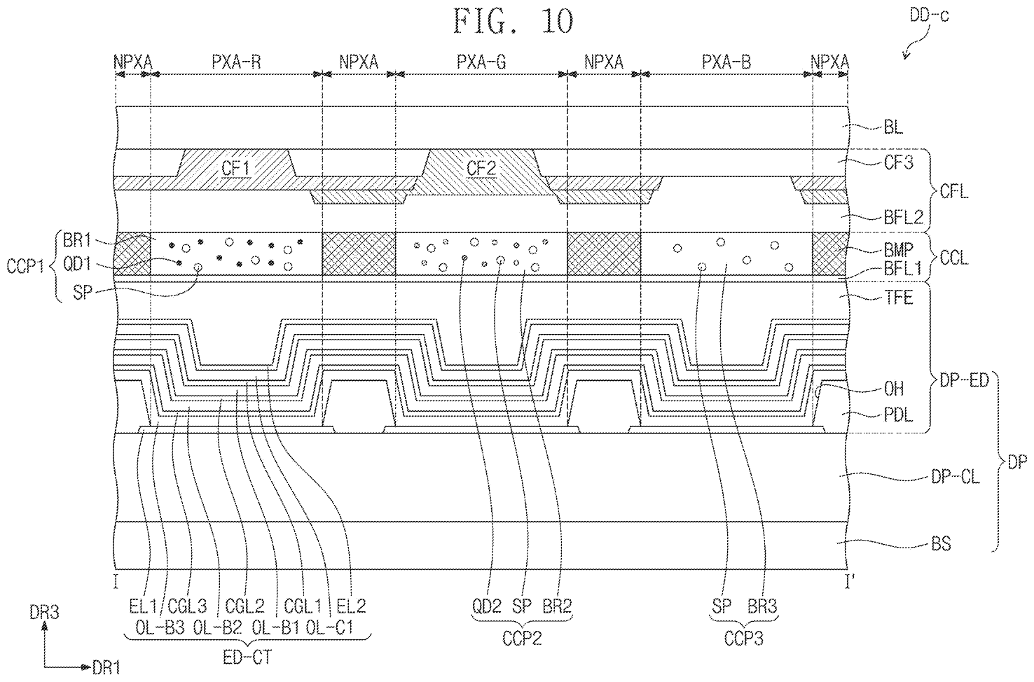

Referring to FIG. 9, a display device DD-b according to an embodiment may include light emitting elements ED-1, ED-2, and ED-3 in which two emission layers are stacked. Compared to the display device DD according to an embodiment shown in FIG. 2, the difference is that in an embodiment shown in FIG. 9, the first to third light emitting elements ED-1, ED-2, and ED-3 each include two emission layers stacked in a thickness direction. In each of the first to third light emitting elements ED-1, ED-2, and ED-3, the two emission layers may emit light in the same wavelength range.

The first light emitting element ED-1 may include a first red emission layer EML-R1 and a second red emission layer EML-R2. The second light emitting element ED-2 may include a first green emission layer EML-G1 and a second green emission layer EML-G2. In addition, the third light emitting element ED-3 may include a first blue emission layer EML-B1 and a second blue emission layer EML-B2. A light emitting auxiliary portion OG may be between the first red emission layer EML-R1 and the second red emission layer EML-R2, between the first green emission layer EML-G1 and the second green emission layer EML-G2, and between the first blue emission layer EML-B1 and the second blue emission layer EML-B2.

The light emitting auxiliary portion OG may include a single layer or a plurality of layers. The light emitting auxiliary portion OG may include a charge generation layer. In one or more embodiments, the light emitting auxiliary portion OG may include an electron transport region, a charge generation layer, and a hole transport region that are sequentially stacked. The light emitting auxiliary portion OG may be provided as a common layer throughout the first to third light emitting elements ED-1, ED-2, and ED-3. However, the embodiment of the present disclosure is not limited thereto, and the light emitting auxiliary portion OG may be provided to be patterned inside the openings OH defined in the pixel defining films PDL.

The first red emission layer EML-R1, the first green emission layer EML-G1, and the first blue emission layer EML-B1 may be between the electron transport region ETR and the emission auxiliary portion OG. The second red emission layer EML-R2, the second green emission layer EML-G2, and the second blue emission layer EML-B2 may be between the emission auxiliary portion OG and the hole transport region HTR.

In one or more embodiments, the light emitting element ED-1 may include the first electrode EL1, the hole transport region HTR, the second red emission layer EML-R2, the emission auxiliary portion OG, the first red emission layer EML-R1, the electron transport region ETR, and the second electrode EL2, which are sequentially stacked. The second light emitting element ED-2 may include the first electrode EL1, the hole transport region HTR, the second green emission layer EML-G2, the emission auxiliary portion OG, the first green emission layer EML-G1, the electron transport region ETR, and the second electrode EL2, which are sequentially stacked. The third light emitting element ED-3 may include the first electrode EL1, the hole transport region HTR, the second blue emission layer EML-B2, the emission auxiliary portion OG, the first blue emission layer EML-B1, the electron transport region ETR, and the second electrode EL2, which are sequentially stacked.

In one or more embodiments, an optical auxiliary layer PL may be on the display element layer DP-ED. The optical auxiliary layer PL may include a polarizing layer. The optical auxiliary layer PL may be on the display panel DP to control reflected light in the display panel DP due to external light. In one or more embodiments, the optical auxiliary layer PL may be omitted from the display device.

At least one emission layer included in a display device DD-b according to an embodiment shown in FIG. 9 may include the nitrogen-containing compound according to an embodiment described above. For example, at least one selected from the first blue emission layer EML-B1 and the second blue emission layer EML-B2 may include the nitrogen-containing compound according to an embodiment.

Unlike FIGS. 8-9, the display device DD-c of FIG. 10 is shown to include four light emitting structures OL-B1, OL-B2, OL-B3, and OL-C1. The light emitting element ED-BT may include the first electrode EL1 and the second electrode EL2 facing each other, and the first to fourth light emitting structures L-B1, OL-B2, OL-B3, and OL-C1 sequentially stacked in a thickness direction between the first electrode EL1 and the second electrode EL2. Charge generation layers CGL1, CGL2, and CGL3 may be between the first to fourth light emitting structures OL-B1, OL-B2, OL-B3, and OL-C1. Among the four light emitting structures, the first to third light emitting structures OL-B1, OL-B2, and OL-B3 may emit blue light, and the fourth light emitting structure OL-C1 may emit green light. However, the embodiment of the present disclosure is not limited thereto, and the first to fourth light emitting structures OL-B1, OL-B2, OL-B3, and OL-C1 may emit light having different wavelength ranges.

The charge generation layers CGL1, CGL2 and CGL3 between the neighboring light emitting structures OL-B1, OL-B2, OL-B3, and OL-C1 may include a p-type charge generation layer and/or an n-type charge generation layer.

At least one selected from the light emitting structures OL-B1, OL-B2, OL-B3, and OL-C1 included in the display device DD-c according to an embodiment may include the nitrogen-containing compound according to an embodiment described above. For example, in an embodiment, at least one selected from the first to third light emitting structures OL-B1, OL-B2, and OL-B3 may include a nitrogen-containing compound according to an embodiment, which is described above.

The light emitting element ED according to an embodiment of the present disclosure includes the nitrogen-containing compound according to an embodiment described above in at least one functional layer between the first electrode EL1 and the second electrode EL2, and may thus exhibit low driving voltage, excellent luminous efficiency, and improved lifespan characteristics. For example, the nitrogen-containing compound according to an embodiment may be included in the emission layer EML of the light emitting element ED according to an embodiment, and the light emitting element according to an embodiment may exhibit low driving voltage, long lifespan, and high efficiency.

Hereinafter, with reference to Examples and Comparative Examples, a nitrogen-containing compound according to an embodiment of the present disclosure and a light emitting element according to an embodiment will be further described. In addition, Examples shown below are shown only for the understanding of the subject matter of the present disclosure, and the scope of the present disclosure is not limited thereto.

### Examples

### 1. Synthesis of Nitrogen-Containing Compounds

First, a process of synthesizing nitrogen-containing compounds according to an embodiment of the present disclosure will be described in more detail by providing a process of synthesizing Compound 1, Compound 18, Compound 40, Compound 62, Compound 90, Compound 102, Compound 546, and Compound 586 as an example. In addition, a process of synthesizing nitrogen-containing compounds, which will be further described hereinafter, is provided as an example, and thus a process of synthesizing nitrogen-containing compounds according to an embodiment of the present disclosure is not limited to Examples below.

### (1) Synthesis of Compound 1

Compound 1 according to an embodiment may be synthesized by, for example, Reaction Formula 1 below.

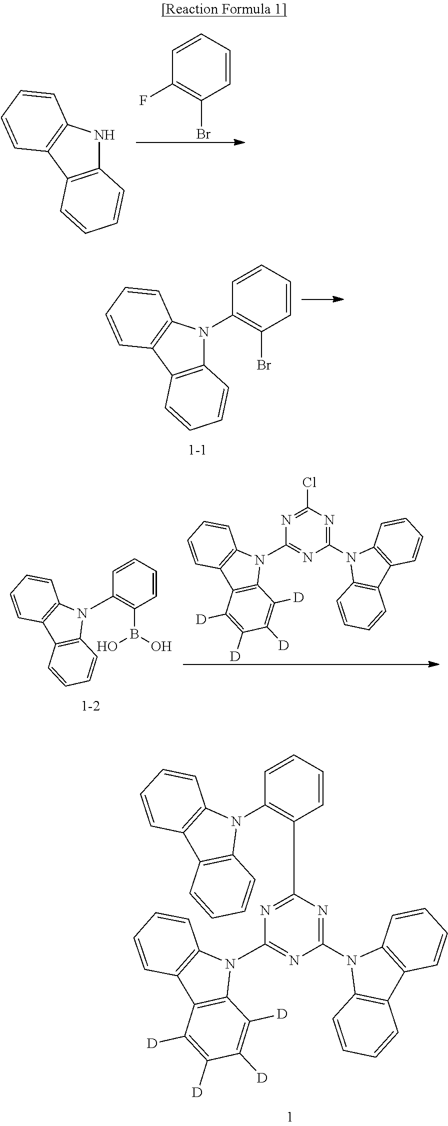

### 1) Synthesis of Intermediate Compound 1-1

9H-carbazole (1 eq.), 1-bromo-2-fluorobenzene (2 eq.), and potassium phosphate tribasic (3 eq.) were dissolved in N,N-dimethylformamide in a nitrogen atmosphere, and then stirred at 160° C. for 12 hours. An obtained solution was cooled to room temperature, washed three times with ethyl acetate and water, and a resulting organic layer was dried over anhydrous magnesium sulfate and then dried under reduced pressure. Thereafter, a resulting product was subjected to separation and purification through column chromatography to obtain Intermediate Compound 1-1 (yield: 92%).

### 2) Synthesis of Intermediate Compound 1-2

Intermediate Compound 1-1 (1 eq.) was dissolved in tetrahydrofuran in a nitrogen atmosphere, and then n-butyllithium solution (2.5 M, 1.2 eq.) was added dropwise at −78° C. and stirred for 1 hour. Thereafter, trimethyl borate (1.3 eq.) was added dropwise, and stirred at room temperature for 12 hours. An obtained solution was washed three times with ethyl acetate and water, and a resulting organic layer was dried over anhydrous magnesium sulfate and then dried under reduced pressure. Then, a resulting product was subjected to separation and purification through column chromatography to obtain Intermediate Compound 1-2 (yield: 71%).

### 3) Synthesis of Compound 1

Intermediate Compound 1-2 (1 eq.), 9-(4-(9H-carbazol-9-yl)-6-chloro-1,3,5-triazin-2-yl)-9H-carbazole-1,2,3,4-d4 (1 eq.), Pd(PPh₃)₄ (0.05 eq.), and K₂CO₃ (3 eq.) were dissolved in a solvent (tetrahydrofuran:H₂O=2:1) in a nitrogen atmosphere, and stirred at 80° C. for 12 hours. An obtained solution was cooled to room temperature, washed three times with ethyl acetate and water, and a resulting organic layer was dried over anhydrous magnesium sulfate and then dried under reduced pressure. Then, a resulting product was subjected to separation and purification through column chromatography to obtain Compound 1 (yield: 73%).

### (2) Synthesis of Compound 18

Compound 18 according to an embodiment may be synthesized by, for example, Reaction Formula 2 below.

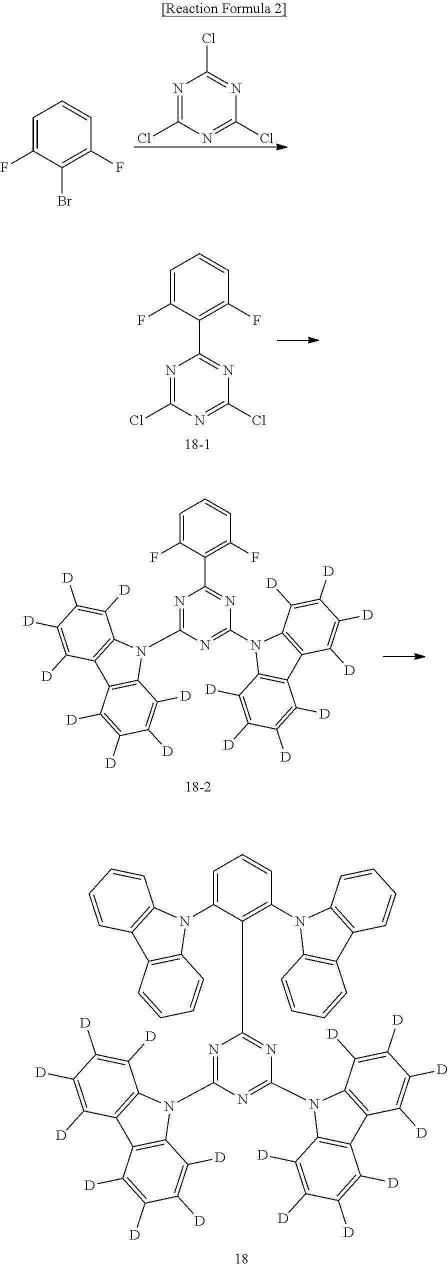

### 1) Synthesis of Intermediate Compound 18-1

2-bromo-1,3-difluorobenzene (1 eq.) was dissolved in tetrahydrofuran in a nitrogen atmosphere, and then n-butyllithium solution (2.5 M, 1.2 eq.) was added dropwise at −78° C. and stirred for 1 hour. Thereafter, trimethyl borate (1.3 eq.) was added dropwise, and stirred at room temperature for 12 hours. An obtained solution was cooled to room temperature, washed three times with ethyl acetate and water, and a resulting organic layer was dried over anhydrous magnesium sulfate and then dried under reduced pressure. Then, a resulting product was subjected to separation and purification through column chromatography to obtain Intermediate Compound 18-1 (yield: 65%).

### 2) Synthesis of Intermediate Compound 18-2

9H-carbazole-1,2,3,4,5,6,7,8-d8 (2.2 eq.) was dissolved in tetrahydrofuran in a nitrogen atmosphere, and then n-butyllithium solution (2.5 M, 2.5 eq.) was added dropwise at 0° C. and stirred for 30 minutes. Thereafter, a solution in which Intermediate Compound 18-1 (1 eq.) was dissolved in tetrahydrofuran was added dropwise, and stirred at 80° C. for 12 hours. An obtained solution was cooled to room temperature, washed three times with ethyl acetate and water, and a resulting organic layer was dried over anhydrous magnesium sulfate and then dried under reduced pressure. Then, a resulting product was subjected to separation and purification through column chromatography to obtain Intermediate Compound 18-2 (yield: 57%).

### 3) Synthesis of Compound 18

9H-carbazole (2.2 eq.) and sodium hydride (4 eq.) were dissolved in N,N-dimethylformamide in a nitrogen atmosphere, and then stirred at room temperature for 20 minutes. Thereafter, a solution in which Intermediate Compound 18-2 (1 eq.) was dissolved in N,N-dimethylformamide was added dropwise, and stirred at 120° C. for 2 hours. An obtained solution was cooled to room temperature, washed three times with ethyl acetate and water, and a resulting organic layer was dried over anhydrous magnesium sulfate and then dried under reduced pressure. Then, a resulting product was subjected to separation and purification through column chromatography to obtain Compound 18 (yield: 37%).

### (3) Synthesis of Compound 40

Compound 40 according to an embodiment may be synthesized by, for example, Reaction Formula 3 below.

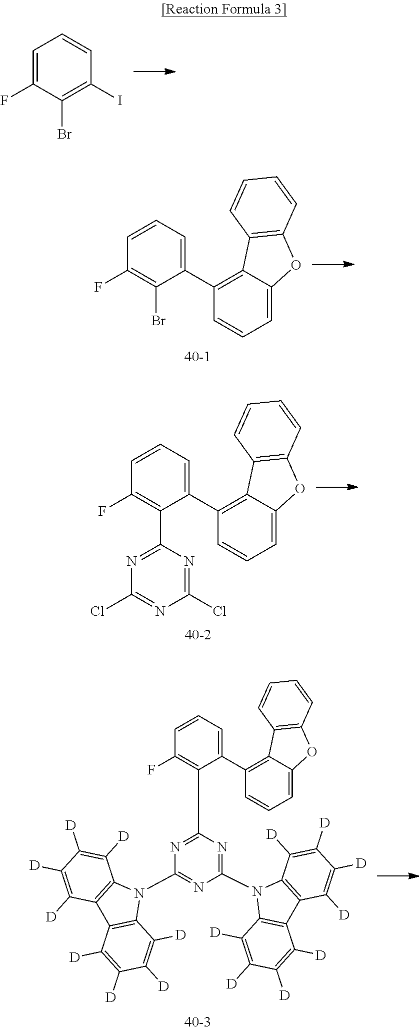 [SMILES: Fc1cccc(I)c1Br ; Fc1cccc(-c2cccc3oc4ccccc4c23)c1Br ; Fc1cccc(-c2cccc3oc4ccccc4c23)c1-c1nc(Cl)nc(Cl)n1 ; [2H]c1c([2H])c([2H])c2c(c1[2H])c1c([2H])c([2H])c([2H])c([2H])c1n2-c1nc(-c2c(F)cccc2-c2cccc3oc4ccccc4c23)nc(-n2c3c([2H])c([2H])c([2H])c([2H])c3c3c([2H])c([2H])c([2H])c([2H])c32)n1]

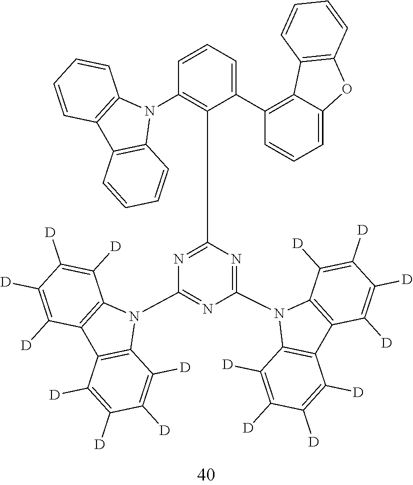 [SMILES: [2H]c1c([2H])c([2H])c2c(c1[2H])c1c([2H])c([2H])c([2H])c([2H])c1n2-c1nc(-c2c(-c3cccc4oc5ccccc5c34)cccc2-n2c3ccccc3c3ccccc32)nc(-n2c3c([2H])c([2H])c([2H])c([2H])c3c3c([2H])c([2H])c([2H])c([2H])c32)n1]

### 1) Synthesis of Intermediate Compound 40-1

2-bromo-1-fluoro-3-iodobenzene (1 eq.), dibenzo[b,d]furan-1-ylboronic acid (1 eq.), Pd(PPh₃)₄ (0.05 eq.), and K₂CO₃ (3 eq.) were dissolved in a solvent (tetrahydrofuran:H₂O=2:1) in a nitrogen atmosphere, and stirred at 80° C. for 12 hours. An obtained solution was cooled to room temperature, washed three times with ethyl acetate and water, and a resulting organic layer was dried over anhydrous magnesium sulfate and then dried under reduced pressure. Then, a resulting product was subjected to separation and purification through column chromatography to obtain Intermediate Compound 40-1 (yield: 53%).

### 2) Synthesis of Intermediate Compound 40-2

Intermediate Compound 40-2 (yield: 48%) was obtained through a reaction under substantially the same conditions as in the method of preparing Intermediate Compound 18-1, except that Intermediate Compound 40-1 was used instead of 2-bromo-1,3-difluorobenzene as a starting material.

### 3) Synthesis of Intermediate Compound 40-3

Intermediate Compound 40-3 (yield: 51%) was obtained through a reaction under substantially the same conditions as in the method of preparing Intermediate Compound 18-2, except that Intermediate Compound 40-2 was used instead of Intermediate Compound 18-1 as a starting material.

### 4) Synthesis of Compound 40

Compound 40 (yield: 57%) was obtained through a reaction under substantially the same conditions as in the method of preparing Compound 18, except that Intermediate Compound 40-3 was used instead of Intermediate Compound 18-2 as a starting material and only 1 equivalent of 9H-carbazole was used. (4) Synthesis of Compound 62

Compound 62 (yield: 45%) was obtained through a reaction under substantially the same conditions as in the method of preparing Compound 40, except that dibenzo[b,d]thiophen-3-ylboronic acid was used instead of dibenzo[b,d]furan-1-ylboronic acid as a starting material.

### (5) Synthesis of Compound 90

Compound 90 (yield: 43%) was obtained through a reaction under substantially the same conditions as in the method of preparing Compound 40, except that (2-(9H-carbazol-9-yl)phenyl)boronic acid was used instead of dibenzo[b,d]furan-1-ylboronic acid as a starting material.

### (6) Synthesis of Compound 102

Compound 102 (yield: 77%) was obtained through a reaction under substantially the same conditions as in the method of preparing Compound 1, except that 9,9′-(2-chloropyrimidine-4,6-diyl)bis(9H-carbazole-1,2,3,4,5,6,7,8-d8 was used instead of 9-(4-(9H-carbazol-9-yl)-6-chloro-1,3,5-triazin-2-yl)-9H-carbazole-1,2,3,4-d4 as a starting material.

### (7) Synthesis of Compound 546

Compound 546 according to an embodiment may be synthesized by, for example, Reaction Formula 4 below.

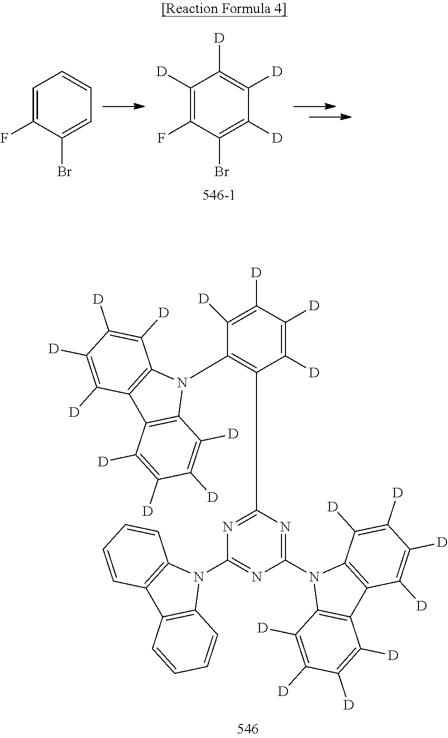 [SMILES: Fc1ccccc1Br ; [2H]c1c([2H])c([2H])c(Br)c(F)c1[2H] ; [2H]c1c([2H])c([2H])c(-n2c3c([2H])c([2H])c([2H])c([2H])c3c3c([2H])c([2H])c([2H])c([2H])c32)c(-c2nc(-n3c4ccccc4c4ccccc43)nc(-n3c4c([2H])c([2H])c([2H])c([2H])c4c4c([2H])c([2H])c([2H])c([2H])c43)n2)c1[2H]]

### (8) Synthesis of Compound 586

### 1) Synthesis of Compound 586

Compound 586 (yield: 36%) was obtained through a reaction under substantially the same conditions as in the method of preparing Compound 18, except that 9H-carbazole-1,2,3,4,5,6,7,8-d8 was used instead of 9H-carbazole as a starting material.

¹H-NMR results of Compound 1, Compound 18, Compound 40, Compound 62, Compound 90, Compound 102, Compound 546, and Compound 586, synthesized through the synthetic method described above are shown in Table 1 below.

TABLE 1

| | | MS/FAB | |
|---|---|---|---|
| Compound | ¹H- NMR (δ) | Calc | Found |
| 1 | δ = 8.55(d, 3H), 8.20(d, 2H), 7.94- | 656.26 | 656.29 |
| | 7.85(m, 6H), 7.59-7.50(m, 5H), 7.37- | | |
| | 7.32(m, 3H), 7.21-7.15(m, 5H) | | |
| 18 | δ = 8.54(d, 2H), 8.19(d, 2H), 7.95- | 833.40 | 833.43 |
| | 7.86(m, 4H), 7.73(t, 1H), 7.58- | | |
| | 7.49(m, 4H), 7.36-7.33(m, 2H), 7.21- | | |
| | 7.18(m, 4H) | | |
| 40 | δ = 8.55(d, 1H), 8.19(d, 1H), 8.03- | 834.38 | 834.34 |
| | 7.92(m, 4H), 7.82-7.69(m, 3H), 7.59- | | |
| | 7.50(m, 4H), 7.39-7.31(m, 3H), 7.20- | | |
| | 7.16(m, 2H) | | |
| 62 | δ = 8.55(d, 1H), 8.45(d, 1H), 8.25- | 850.36 | 850.38 |
| | 8.17(m, 4H), 8.02-7.92(m, 4H), | | |
| | 7.79(t, 1H), 7.58-7.49(m, 4H), 7.35(t, | | |
| | 1H), 7.21-7.16(m, 2H) | | |
| 90 | δ = 8.55(d, 4H), 8.19(d, 2H), 8.02- | 901.38 | 901.33 |
| | 7.91(m, 8H), 7.80-7.77(m, 2H), 7.58- | | |
| | 7.45(m, 5H), 7.37-7.33(m, 4H), 7.21- | | |
| | 7.16(m, 6H) | | |
| 102 | δ = 8.54(d, 1H), 8.20(d, 1H), 7.94- | 667.34 | 667.30 |
| | 7.91(m, 3H), 7.81-7.78(m, 1H), 7.58- | | |
| | 7.47(m, 3H), 7.36(t, 1H), 7.20- | | |
| | 7.16(m, 3H) | | |
| 546 | δ = 8.53(d, 1H), 8.18(d, 1H), 7.93(d, | 672.36 | 672.39 |
| | 1H), 7.58-7.51(m, 2H), 7.36(t, 1H), | | |
| | 7.21-7.17(m, 2H) | | |
| 586 | δ = 7.97(d, 2H), 7.94(d, 1H) | 849.50 | 849.54 |

### 2. Preparation and Evaluation of Light Emitting Elements

### (1) Preparation of Light Emitting Elements

Light emitting elements including nitrogen-containing compounds according to an embodiment or Comparative Example compounds were prepared through a method below. Light emitting elements of Examples 1 to 8 were prepared respectively using nitrogen-containing compounds according to an embodiment, Compound 1, Compound 18, Compound 40, Compound 62, Compound 90, Compound 102, Compound 546, and Compound 586 as a host material of an emission layer. Light emitting elements of Comparative Examples 1 and 2 were prepared respectively using Comparative Example Compounds A and B as a host material of an emission layer.

An ITO glass substrate (corning, 15 Ω/cm², 1200 Å) was cut to a size of about 50 mm×50 mm×0.5 mm, subjected to ultrasonic cleaning using isopropyl alcohol and pure water for 5 minutes respectively and ultraviolet irradiation for 30 minutes, and then exposed to ozone for cleaning to form the ITO glass substrate as a first electrode in a vacuum deposition apparatus.

HAT-CN was formed to have a thickness of 100 Å as a hole injection layer on the first electrode, and then BCFN, a first hole transport material, was vacuum-deposited to have a thickness of 600 Å, and SiCzCz, a second hole transport material, was vacuum-deposited to have a thickness of 50 Å as a second hole transporting compound to form a hole transport layer.

SiCzCz as a host, Example compound (or Comparative Example compound), and a phosphorescent dopant PtON-TBBI were co-deposited in a weight ratio of 60:27:13 on an upper portion of the hole transport layer to form an emission layer having a thickness of 350 Å.

Thereafter, Example compound or Comparative Example compound was deposited to have a thickness of 50 Å as an auxiliary layer on an upper portion of the emission layer, and then mSiTrz and LiQ were co-deposited in a ratio of 1:1 to form an electron transport layer having a thickness of 350 Å. An alkali metal halide, LiF, was deposited on an upper portion of the electron transport layer to have a thickness of 15 Å as an electron injection layer, and Al was vacuum-deposited to have a thickness of 80 Å to form a LiF/Al electrode, thereby manufacturing a light emitting element.

### [Compounds Used Upon Preparation of Light Emitting Elements]

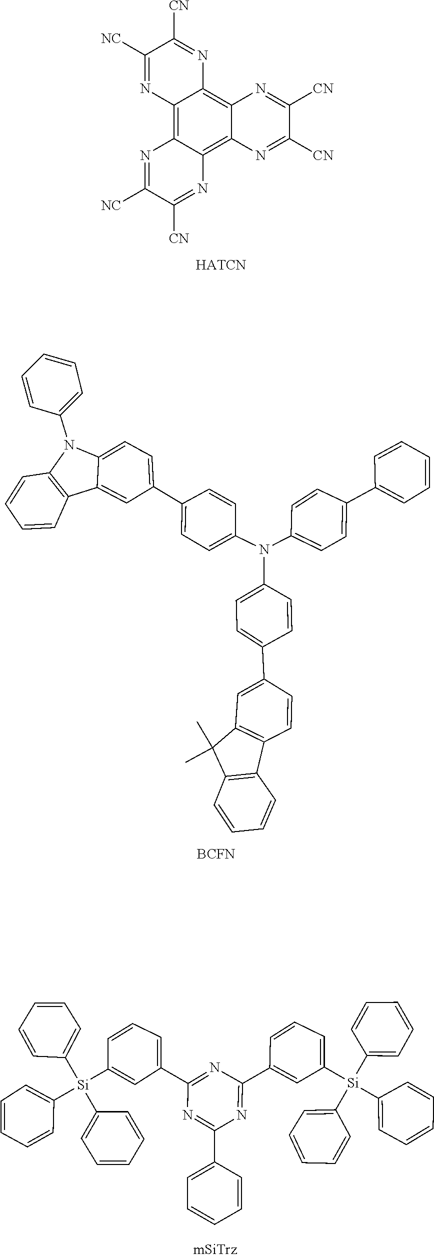

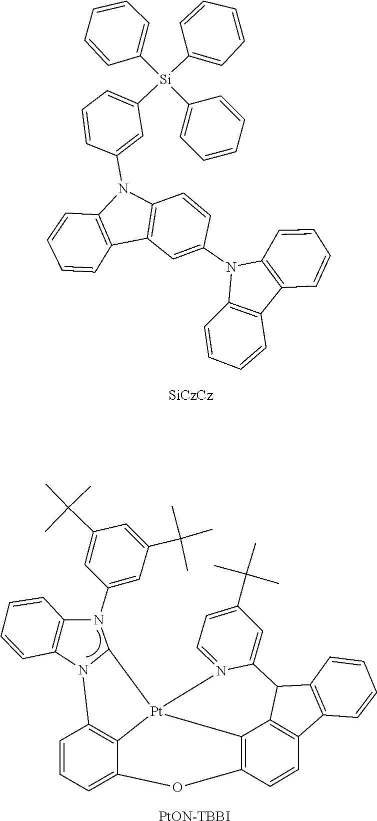

### [Example Compound and Comparative Example Compound>

Example Compounds used in Examples 1 to 8 and Comparative Example Compounds used in Comparative Examples 1 and 2 are shown in Table 2.

TABLE 2

| | |
|---|---|
| Example Compound 1 | 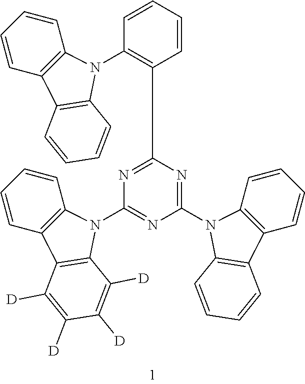 [SMILES: [2H]c1c([2H])c([2H])c2c(c1[2H])c1ccccc1n2-c1nc(-c2ccccc2-n2c3ccccc3c3ccccc32)nc(-n2c3ccccc3c3ccccc32)n1] |
| Example Compound 18 | 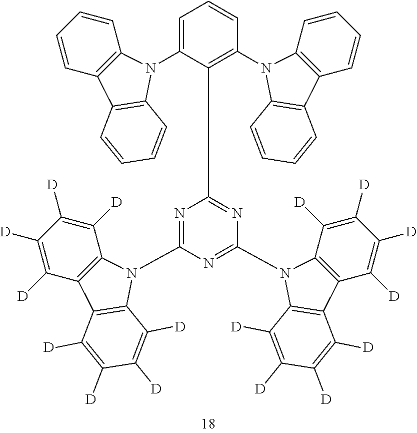 [SMILES: [2H]c1c([2H])c([2H])c2c(c1[2H])c1c([2H])c([2H])c([2H])c([2H])c1n2-c1nc(-c2c(-n3c4ccccc4c4ccccc43)cccc2-n2c3ccccc3c3ccccc32)nc(-n2c3c([2H])c([2H])c([2H])c([2H])c3c3c([2H])c([2H])c([2H])c([2H])c32)n1] |
| Example Compound 40 | 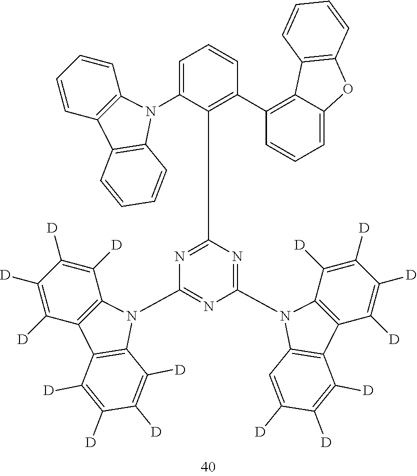 [SMILES: [2H]c1cc2c(c([2H])c1[2H])c1c([2H])c([2H])c([2H])c([2H])c1n2-c1nc(-c2c(-c3cccc4oc5ccccc5c34)cccc2-n2c3ccccc3c3ccccc32)nc(-n2c3c([2H])c([2H])c([2H])c([2H])c3c3c([2H])c([2H])c([2H])c([2H])c32)n1] |
| Example Compound 62 | 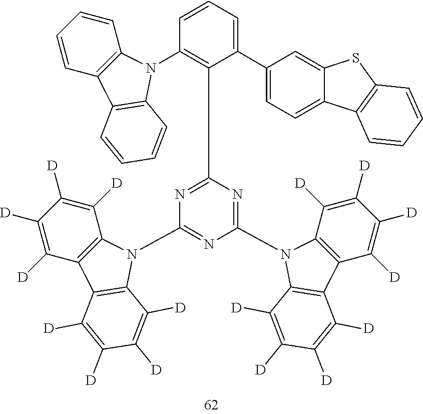 [SMILES: [2H]c1c([2H])c([2H])c2c(c1[2H])c1c([2H])c([2H])c([2H])c([2H])c1n2-c1nc(-c2c(-c3ccc4c(c3)sc3ccccc34)cccc2-n2c3ccccc3c3ccccc32)nc(-n2c3c([2H])c([2H])c([2H])c([2H])c3c3c([2H])c([2H])c([2H])c([2H])c32)n1] |
| Example Compound 90 | 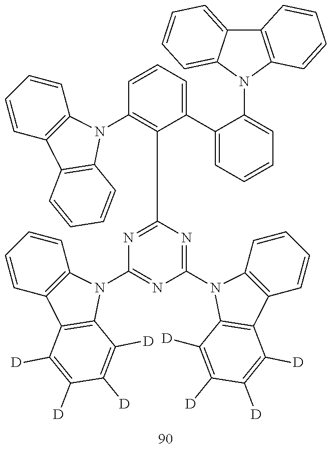 [SMILES: [2H]c1c([2H])c([2H])c2c(c1[2H])c1ccccc1n2-c1nc(-c2c(-c3ccccc3-n3c4ccccc4c4ccccc43)cccc2-n2c3ccccc3c3ccccc32)nc(-n2c3ccccc3c3c([2H])c([2H])c([2H])c([2H])c32)n1] |
| Example Compound 102 | 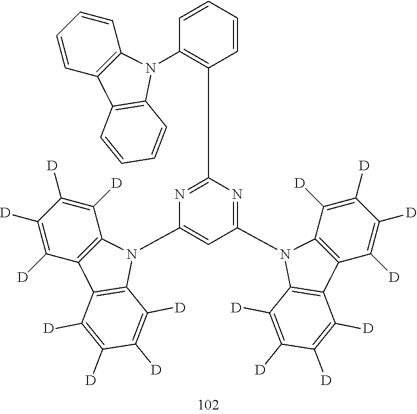 [SMILES: [2H]c1c([2H])c([2H])c2c(c1[2H])c1c([2H])c([2H])c([2H])c([2H])c1n2-c1cc(-n2c3c([2H])c([2H])c([2H])c([2H])c3c3c([2H])c([2H])c([2H])c([2H])c32)nc(-c2ccccc2-n2c3ccccc3c3ccccc32)n1] |
| Example Compound 546 | 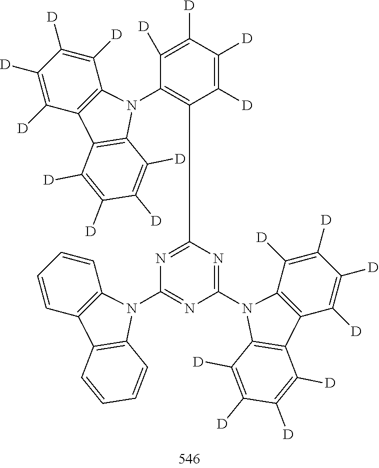 [SMILES: [2H]c1c([2H])c([2H])c(-n2c3c([2H])c([2H])c([2H])c([2H])c3c3c([2H])c([2H])c([2H])c([2H])c32)c(-c2nc(-n3c4ccccc4c4ccccc43)nc(-n3c4c([2H])c([2H])c([2H])c([2H])c4c4c([2H])c([2H])c([2H])c([2H])c43)n2)c1[2H]] |
| Example Compound 592 | 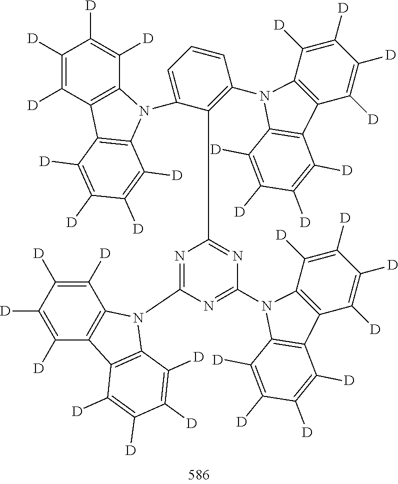 [SMILES: [2H]c1c([2H])c([2H])c2c(c1[2H])c1c([2H])c([2H])c([2H])c([2H])c1n2-c1nc(-c2c(-n3c4c([2H])c([2H])c([2H])c([2H])c4c4c([2H])c([2H])c([2H])c([2H])c43)cccc2-n2c3c([2H])c([2H])c([2H])c([2H])c3c3c([2H])c([2H])c([2H])c([2H])c32)nc(-n2c3c([2H])c([2H])c([2H])c([2H])c3c3c([2H])c([2H])c([2H])c([2H])c32)n1] |
| Comparative Example Compound A | 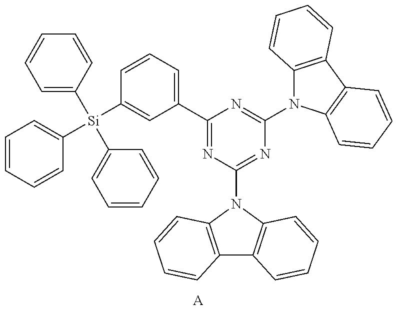 |
| Comparative Example Compound B | 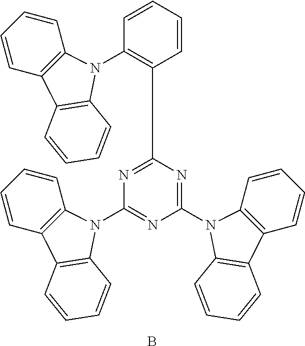 |

### (2) Characteristics Evaluation of Light Emitting Elements

Table 3 shows evaluation results of driving voltage, maximum quantum efficiency, half-life, and emitted color of the light emitting elements of Examples and Comparative Examples. The driving voltage, maximum quantum efficiency, and half-life were evaluated at a current density of 10 mA/cm². The driving voltage was determined using a source meter (2400 series from Keithley Instrument), and the maximum quantum efficiency was determined using an external quantum efficiency measuring apparatus (C9920-2-12 from Hamamatsu Photonics Co., Ltd.) In the evaluation of the maximum quantum efficiency, the luminance and current densities were measured using a luminance meter that was calibrated for wavelength sensitivity, and the maximum quantum efficiency was converted under the assumption that an angular luminance distribution (Lambertian) was obtained with respect to a fully diffused reflective surface.

TABLE 3

| | | | Maximum | Half life | |
|---|---|---|---|---|---|
| | Light | Driving | quantum | (hr @ | |
| | emitting | voltage | efficiency | 10 mA/ | Emitted |
| | material | (V) | (%) | cm²) | color |
| Example 1 | Compound 1 | 3.6 | 17.0 | 76 | Blue |
| Example 2 | Compound 18 | 3.4 | 18.2 | 85 | Blue |
| Example 3 | Compound 40 | 3.2 | 17.4 | 80 | Blue |
| Example 4 | Compound 62 | 3.3 | 17.8 | 84 | Blue |
| Example 5 | Compound 90 | 3.6 | 18.7 | 89 | Blue |
| Example 6 | Compound 102 | 3.2 | 19.0 | 93 | Blue |
| Example 7 | Compound 546 | 3.3 | 19.6 | 91 | Blue |
| Example 8 | Compound 586 | 3.4 | 19.9 | 107 | Blue |
| Comparative | Compound A | 3.8 | 15.9 | 79 | Blue |
| Example 1 | | | | | |
| Comparative | Compound B | 3.9 | 14.7 | 70 | Blue |
| Example 2 | | | | | |

Referring to Table 3, it can be seen that, compared to the light emitting elements of Comparative Examples 1 and 2, the light emitting elements of Examples 1 to 8 had reduced driving voltage, and excellent light efficiency, maximum quantum efficiency, and lifespan.

A light emitting element of an embodiment may exhibit improved element characteristics such as low driving voltage, high efficiency, and long service life.

A nitrogen-containing compound of an embodiment may be included in an emission layer of a light emitting element to contribute to low driving voltage, increased luminous efficiency, and longer service life of the light emitting element.

Although the subject matter of the present disclosure has been described with reference to example embodiments of the present disclosure, it will be understood that the subject matter of the present disclosure should not be limited to these disclosure embodiments but various changes and modifications can be made by those skilled in the art without departing from the spirit and scope of the present disclosure.

Accordingly, the technical scope of the present disclosure is not intended to be limited to the contents set forth in the detailed description of the specification, but is intended to be defined by the appended claims, and equivalents thereof.

What is claimed is:

1. A light emitting element comprising:
a first electrode;
a second electrode on the first electrode; and
an emission layer between the first electrode and the second electrode, and comprising a first compound represented by Formula 1 below: 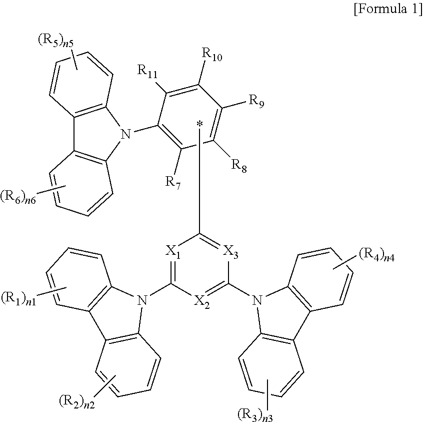
wherein in Formula 1 above,
at least one selected from X₁ to X₃ is N, and the others are each independently CRₓ,
RₓX is a hydrogen atom, a deuterium atom, a substituted or unsubstituted alkyl group having 1 to 60 carbon atoms, a substituted or unsubstituted aryl group having 6 to 60 ring-forming carbon atoms, or a substituted or unsubstituted heteroaryl group having 2 to 60 ring-forming carbon atoms,
at least one selected from R₁ to R₁₁ is a deuterium atom, an unsubstituted carbazole group, a deuterium atom-substituted or unsubstituted phenyl group, an unsubstituted dibenzofuran group, an unsubstituted dibenzothiophene group, or an unsubstituted 9-phenylcarbazole group, and the others are each independently a hydrogen atom, a cyano group, a substituted or unsubstituted alkyl group having 1 to 60 carbon atoms, a substituted or unsubstituted aryl group having 6 to 60 ring-forming carbon atoms, or a substituted or unsubstituted heteroaryl group having 2 to 60 ring-forming carbon atoms, or bonded to an adjacent group to form a ring,
n1 to n6 are each independently an integer of 0 to 4,
“-*” is connected to any one selected from among R₇ to R₁₁, and
a case where X₁ to X₃ are all N, “-*” is connected to R₇, R₈ is an unsubstituted carbazole group, and R₁ to R₆ and R₉ to R₁₁ are all hydrogen atoms is excluded.

2. The light emitting element of claim 1, wherein the first compound represented by Formula 1 above is represented by Formula 2 below: 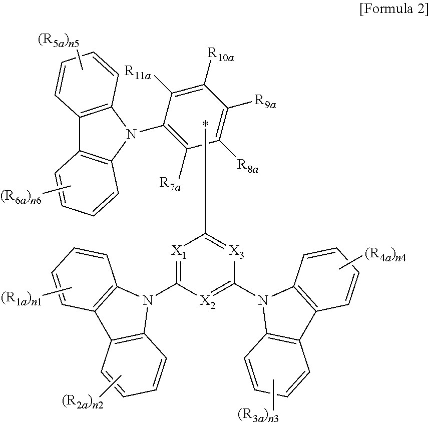
wherein in Formula 2 above,
at least one selected from R₁ₐ to R₁₁ₐ is a deuterium atom, an unsubstituted carbazole group, a deuterium atom-substituted or unsubstituted phenyl group, an unsubstituted dibenzofuran group, an unsubstituted dibenzothiophene group, or an unsubstituted 9-phenylcarbazole group, and the others are hydrogen atoms,
“-*” is connected to any one selected from among R₇ₐ to R₁₁ₐ, and
X₁ to X₃, and n1 to n6 are the same as defined with respect to Formula 1 above.

3. The light emitting element of claim 2, wherein the first compound represented by Formula 2 above is represented by any one selected from among Formulas 2-a to 2-d below: 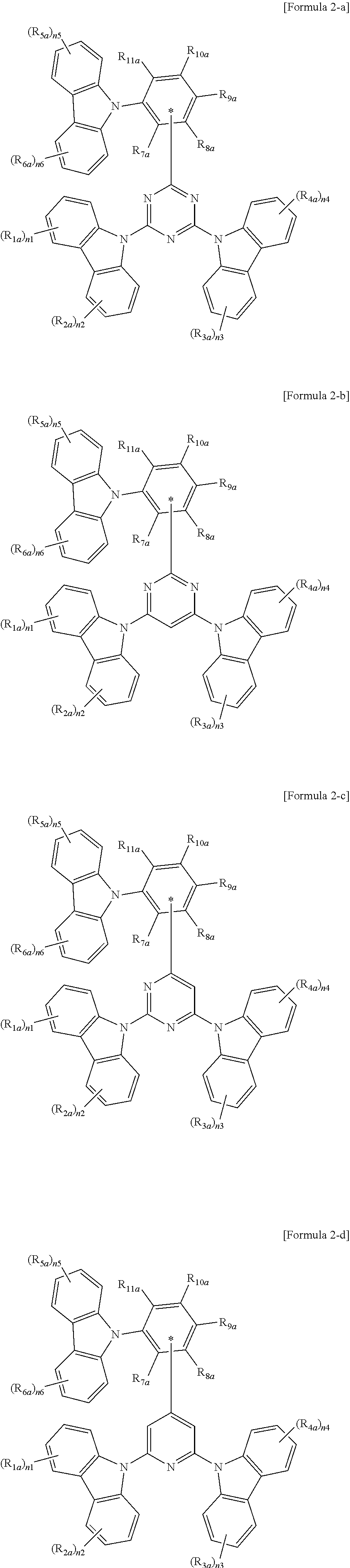
wherein in Formulas 2-a to 2-d above,
R₁ₐ to R₁₁ₐ, n1 to n6, and “-*” are the same as defined with respect to Formula 1 and Formula 2 above.

4. The light emitting element of claim 1, wherein the first compound represented by Formula 1 above is represented by Formula 3-1 or Formula 3-2 below: 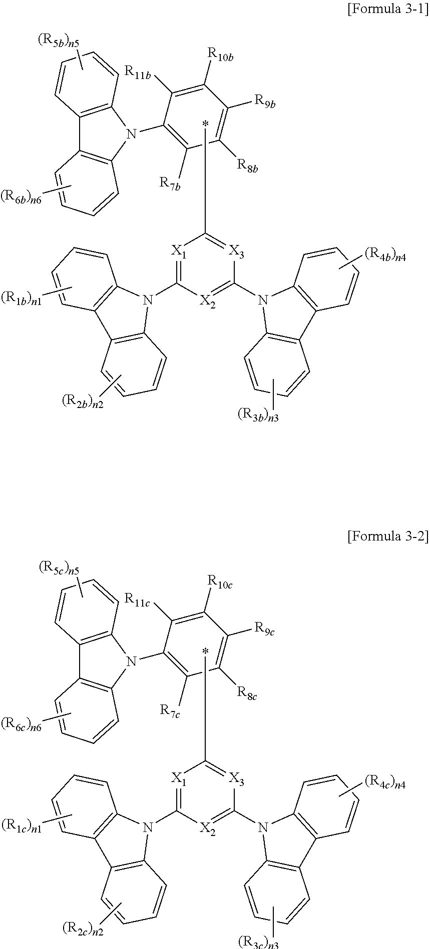
wherein in Formula 3-1 above,
at least one selected from R_{1b} to R_{11b} is a deuterium atom, and the others are each independently a hydrogen atom, a cyano group, a substituted or unsubstituted alkyl group having 1 to 60 carbon atoms, a substituted or unsubstituted aryl group having 6 to 60 ring-forming carbon atoms, or a substituted or unsubstituted heteroaryl group having 2 to 60 ring-forming carbon atoms, or bonded to an adjacent group to form a ring, and
“-*” is connected to any one selected from among R_{7b} to R_{11b},
wherein in Formula 3-2 above,
R_{1c} to R_{6c} are each independently a hydrogen atom, a deuterium atom, a cyano group, a substituted or unsubstituted alkyl group having 1 to 60 carbon atoms, a substituted or unsubstituted aryl group having 6 to 60 ring-forming carbon atoms, or a substituted or unsubstituted heteroaryl group having 2 to 60 ring-forming carbon atoms, or bonded to an adjacent group to form a ring,
at least one selected from R_{7c} to R_{11c} is an unsubstituted carbazole group, a deuterium atom-substituted or unsubstituted phenyl group, an unsubstituted dibenzofuran group, an unsubstituted dibenzothiophene group, or an unsubstituted 9-phenylcarbazole group, and the others are each independently a hydrogen atom or a deuterium atom, and
“-*” is connected to any one selected from among R_{7c} to R_{11c}, and
wherein in Formulas 3-1 and 3-2 above,
X₁ to X₃, and n1 to n6 are the same as defined with respect to Formula 1 above.

5. The light emitting element of claim 4, wherein at least one selected from R_{1b} to R_{11b} is a deuterium atom, and the others are hydrogen atoms.

6. The light emitting element of claim 4, wherein the first compound represented by Formula 3-2 above is represented by any one selected from among Formulas 3-2a to 3-2c below: 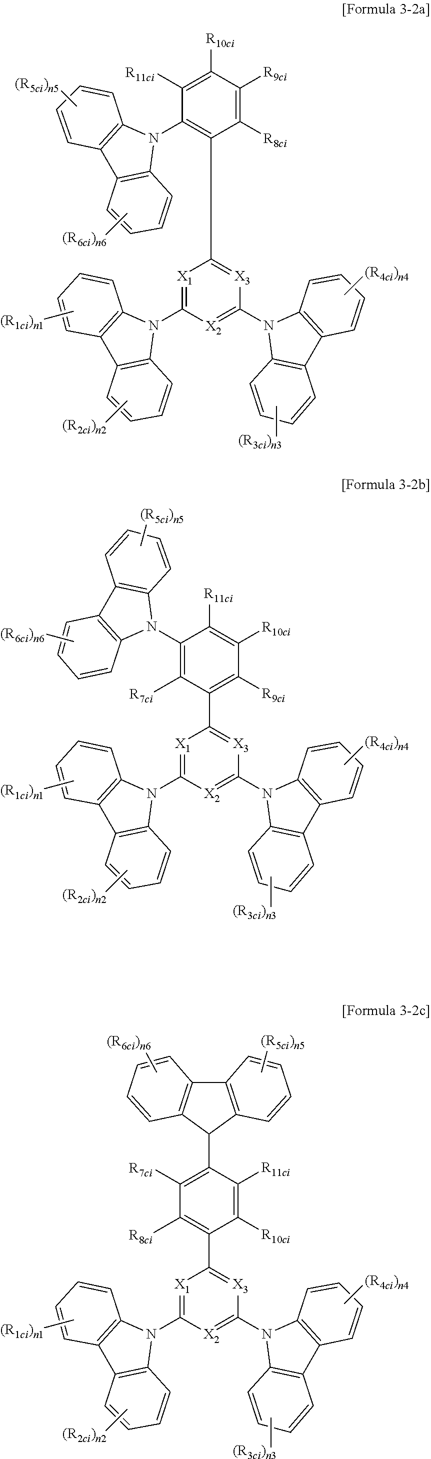
wherein in Formula 3-2a above,
any one among R_{8ci} to R_{11ci} is an unsubstituted carbazole group, a deuterium atom-substituted or unsubstituted phenyl group, an unsubstituted dibenzofuran group, an unsubstituted dibenzothiophene group, or an unsubstituted 9-phenylcarbazole group, and the others are each independently a hydrogen atom or a deuterium atom,
wherein in Formula 3-2b above,
any one among R_{7ci}, and R_{9ci} to R_{11ci} is an unsubstituted carbazole group, a deuterium atom-substituted or unsubstituted phenyl group, an unsubstituted dibenzofuran group, an unsubstituted dibenzothiophene group, or an unsubstituted 9-phenylcarbazole group, and the others are each independently a hydrogen atom or a deuterium atom,
wherein in Formula 3-2c above,
any one among R_{7ci}, R_{8ci}, R_{10ci}, and R_{11ci} is an unsubstituted carbazole group, a deuterium atom-substituted or unsubstituted phenyl group, an unsubstituted dibenzofuran group, an unsubstituted dibenzothiophene group, or an unsubstituted 9-phenylcarbazole group, and the others are each independently a hydrogen atom or a deuterium atom, and
wherein in Formulas 3-2a to 3-2c above,
R_{1ci} to R_{6ci} are each independently a hydrogen atom or a deuterium atom, and
X₁ to X₃, and n1 to n6 are the same as defined with respect to Formula 3-2 above.

7. The light emitting element of claim 1, wherein the first compound represented by Formula 1 above is represented by Formula 4 below: 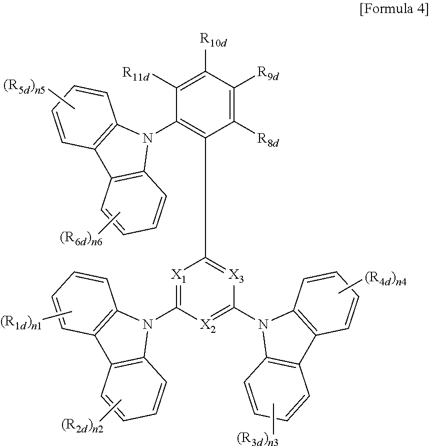
wherein in Formula 4 above,
R_{1d} to R_{6d} are each independently a hydrogen atom or a deuterium atom,
any one among R_{8d} to R_{11d} is a deuterium atom, an unsubstituted carbazole group, a deuterium atom-substituted or unsubstituted phenyl group, an unsubstituted dibenzofuran group, an unsubstituted dibenzothiophene group, or an unsubstituted 9-phenylcarbazole group, and the others are each independently a hydrogen atom or a deuterium atom, and
X₁ to X₃, and n1 to n6 are the same as defined with respect to Formula 1 above.

8. The light emitting element of claim 1, wherein Rₓ is a hydrogen atom or a deuterium atom.

9. The light emitting element of claim 1, wherein in Formula 1 above, at least one selected from R₁ to R₁₁ is a deuterium atom or a substituent containing a deuterium atom.

10. The light emitting element of claim 1, wherein the emission layer comprises a host and a dopant,
the host containing the first compound.

11. The light emitting element of claim 1, wherein the emission layer emits delayed fluorescence or phosphorescence.

12. The light emitting element of claim 1, wherein the first compound 1 is represented by any one selected from among compounds of Compound Group 1 below:

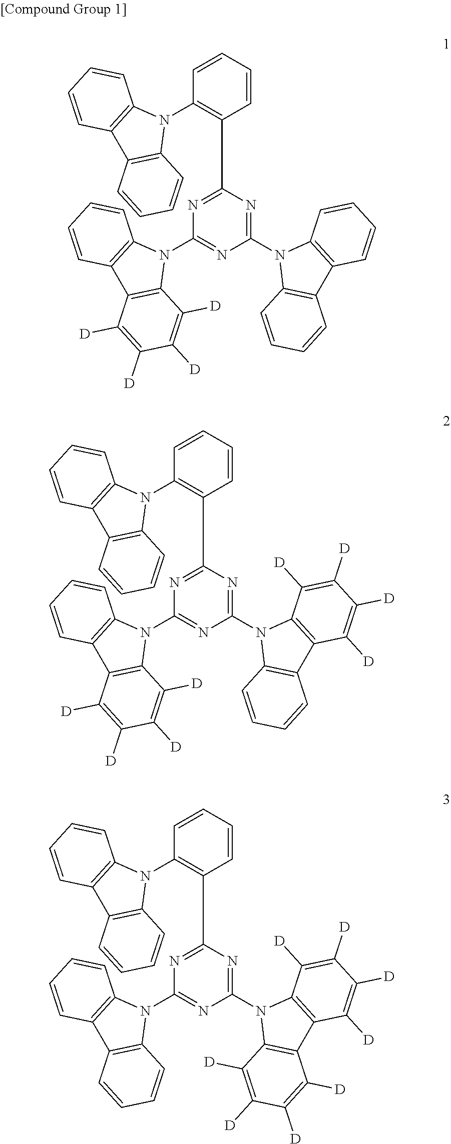 [SMILES: [2H]c1c([2H])c([2H])c2c(c1[2H])c1ccccc1n2-c1nc(-c2ccccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2ccccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1c([2H])c([2H])c([2H])c([2H])c1n2-c1nc(-c2ccccc2-n2c3ccccc3c3ccccc32)nc(-n2c3ccccc3c3ccccc32)n1]

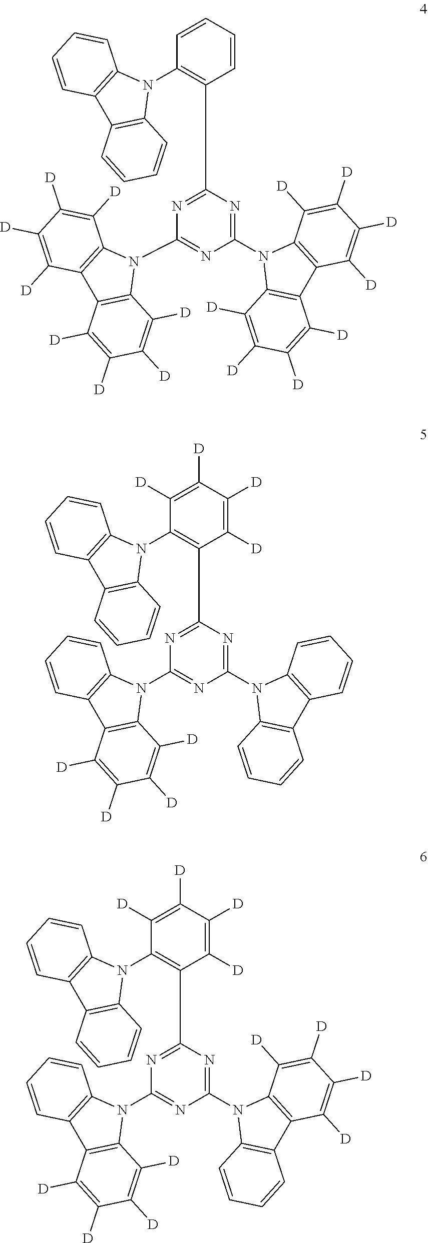 [SMILES: [2H]c1c([2H])c([2H])c2c(c1[2H])c1c([2H])c([2H])c([2H])c([2H])c1n2-c1nc(-c2ccccc2-n2c3ccccc3c3ccccc32)nc(-n2c3c([2H])c([2H])c([2H])c([2H])c3c3c([2H])c([2H])c([2H])c([2H])c32)n1 ; [2H]c1c([2H])c([2H])c(-n2c3ccccc3c3ccccc32)c(-c2nc(-n3c4ccccc4c4ccccc43)nc(-n3c4ccccc4c4c([2H])c([2H])c([2H])c([2H])c43)n2)c1[2H] ; [2H]c1c([2H])c([2H])c(-n2c3ccccc3c3ccccc32)c(-c2nc(-n3c4ccccc4c4c([2H])c([2H])c([2H])c([2H])c43)nc(-n3c4ccccc4c4c([2H])c([2H])c([2H])c([2H])c43)n2)c1[2H]]

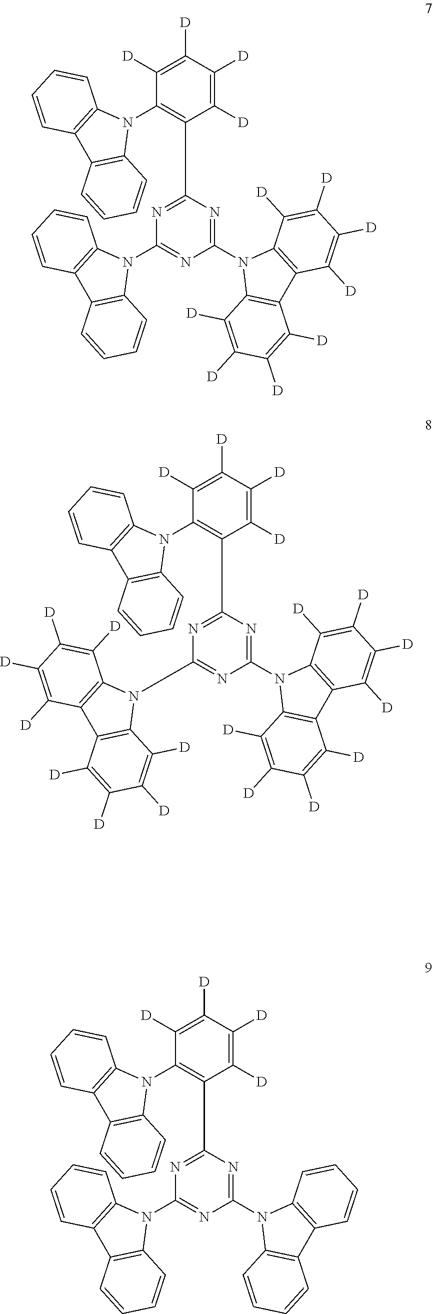 [SMILES: [2H]c1c([2H])c([2H])c(-n2c3ccccc3c3ccccc32)c(-c2nc(-n3c4ccccc4c4ccccc43)nc(-n3c4c([2H])c([2H])c([2H])c([2H])c4c4c([2H])c([2H])c([2H])c([2H])c43)n2)c1[2H] ; [2H]c1c([2H])c([2H])c(-n2c3ccccc3c3ccccc32)c(-c2nc(-n3c4c([2H])c([2H])c([2H])c([2H])c4c4c([2H])c([2H])c([2H])c([2H])c43)nc(-n3c4c([2H])c([2H])c([2H])c([2H])c4c4c([2H])c([2H])c([2H])c([2H])c43)n2)c1[2H] ; [2H]c1c([2H])c([2H])c(-n2c3ccccc3c3ccccc32)c(-c2nc(-n3c4ccccc4c4ccccc43)nc(-n3c4ccccc4c4ccccc43)n2)c1[2H]]

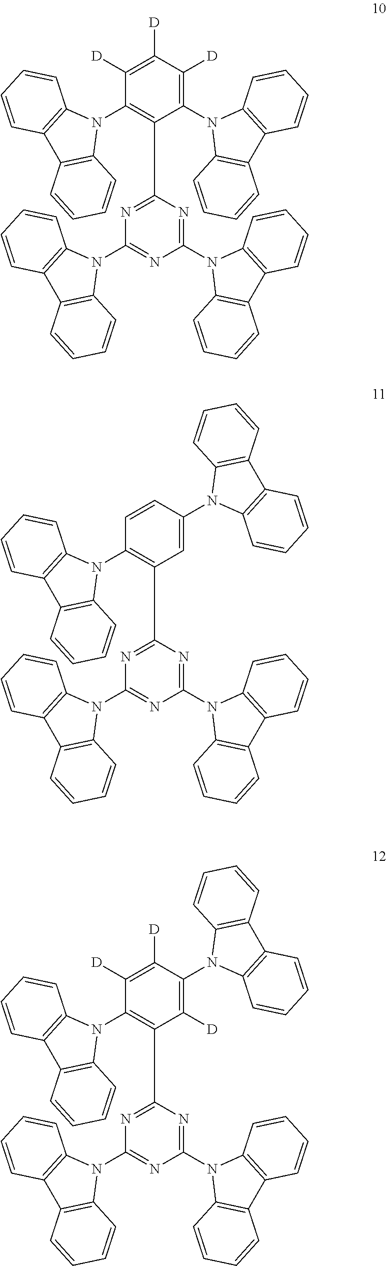

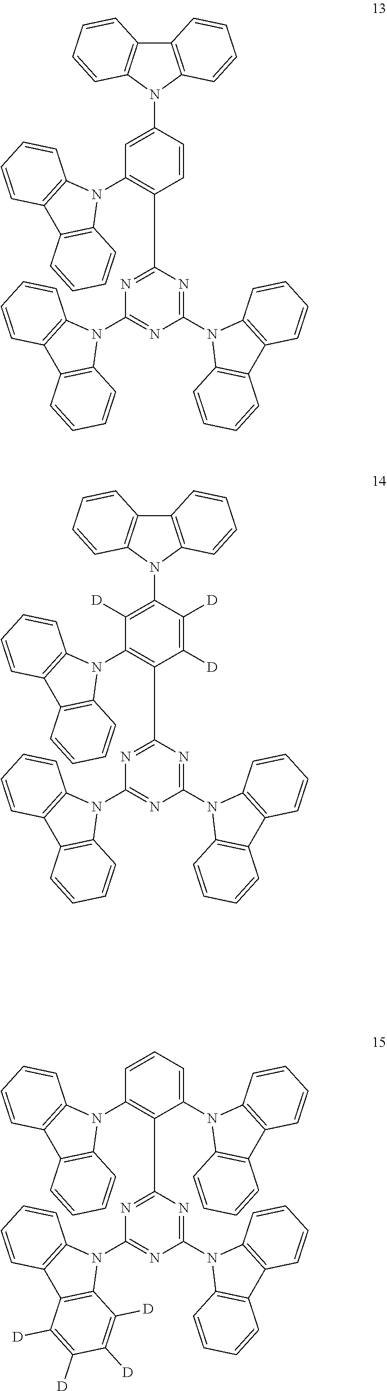 [SMILES: c1ccc2c(c1)c1ccccc1n2-c1ccc(-c2nc(-n3c4ccccc4c4ccccc43)nc(-n3c4ccccc4c4ccccc43)n2)c(-n2c3ccccc3c3ccccc32)c1 ; [2H]c1c([2H])c(-n2c3ccccc3c3ccccc32)c([2H])c(-n2c3ccccc3c3ccccc32)c1-c1nc(-n2c3ccccc3c3ccccc32)nc(-n2c3ccccc3c3ccccc32)n1 ; [2H]c1c([2H])c([2H])c2c(c1[2H])c1ccccc1n2-c1nc(-c2c(-n3c4ccccc4c4ccccc43)cccc2-n2c3ccccc3c3ccccc32)nc(-n2c3ccccc3c3ccccc32)n1]

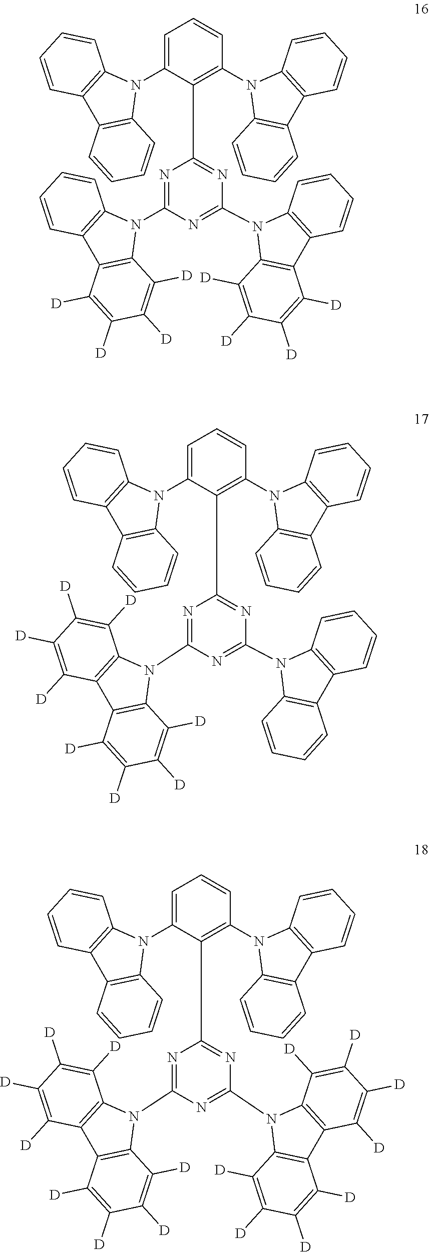

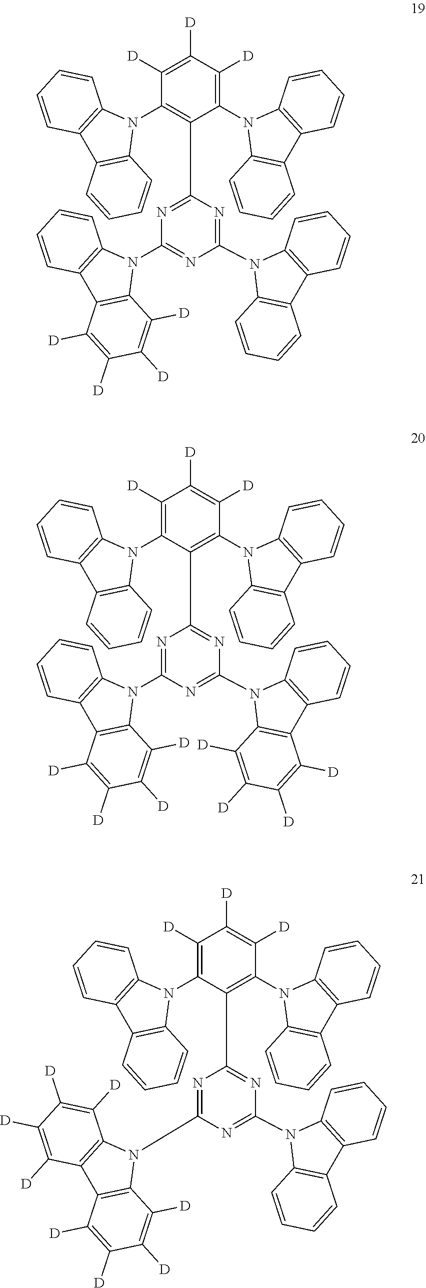 [SMILES: [2H]c1c([2H])c(-n2c3ccccc3c3ccccc32)c(-c2nc(-n3c4ccccc4c4ccccc43)nc(-n3c4ccccc4c4c([2H])c([2H])c([2H])c([2H])c43)n2)c(-n2c3ccccc3c3ccccc32)c1[2H] ; [2H]c1c([2H])c(-n2c3ccccc3c3ccccc32)c(-c2nc(-n3c4ccccc4c4c([2H])c([2H])c([2H])c([2H])c43)nc(-n3c4ccccc4c4c([2H])c([2H])c([2H])c([2H])c43)n2)c(-n2c3ccccc3c3ccccc32)c1[2H] ; [2H]c1c([2H])c(-n2c3ccccc3c3ccccc32)c(-c2nc(-n3c4ccccc4c4ccccc43)nc(-n3c4c([2H])c([2H])c([2H])c([2H])c4c4c([2H])c([2H])c([2H])c([2H])c43)n2)c(-n2c3ccccc3c3ccccc32)c1[2H]]

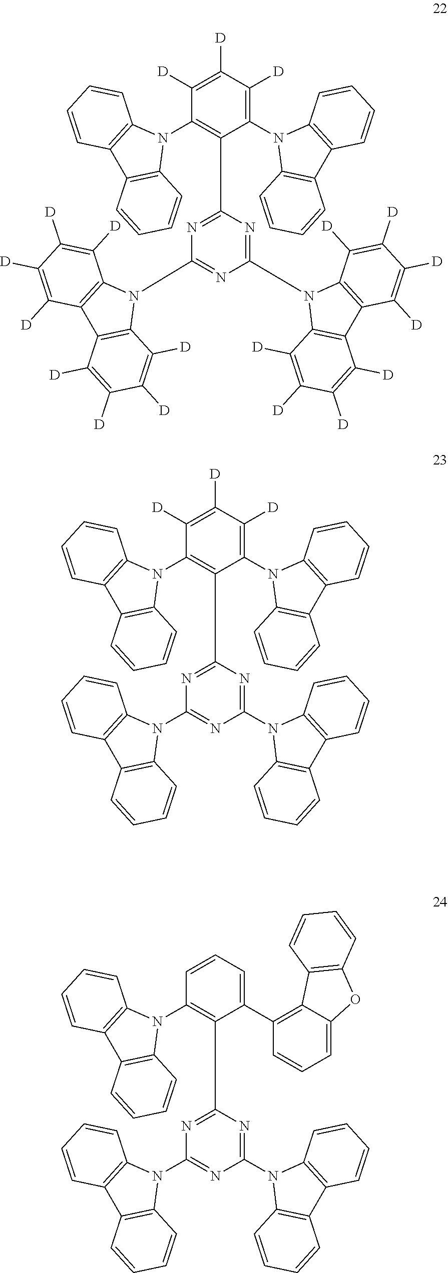 [SMILES: [2H]c1c([2H])c(-n2c3ccccc3c3ccccc32)c(-c2nc(-n3c4c([2H])c([2H])c([2H])c([2H])c4c4c([2H])c([2H])c([2H])c([2H])c43)nc(-n3c4c([2H])c([2H])c([2H])c([2H])c4c4c([2H])c([2H])c([2H])c([2H])c43)n2)c(-n2c3ccccc3c3ccccc32)c1[2H] ; [2H]c1c([2H])c(-n2c3ccccc3c3ccccc32)c(-c2nc(-n3c4ccccc4c4ccccc43)nc(-n3c4ccccc4c4ccccc43)n2)c(-n2c3ccccc3c3ccccc32)c1[2H] ; c1cc(-c2cccc3oc4ccccc4c23)c(-c2nc(-n3c4ccccc4c4ccccc43)nc(-n3c4ccccc4c4ccccc43)n2)c(-n2c3ccccc3c3ccccc32)c1]

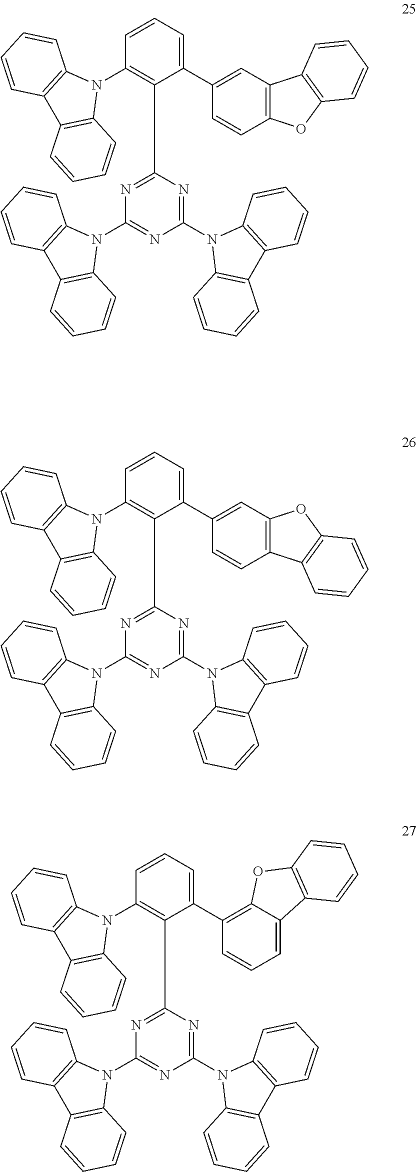 [SMILES: c1cc(-c2ccc3oc4ccccc4c3c2)c(-c2nc(-n3c4ccccc4c4ccccc43)nc(-n3c4ccccc4c4ccccc43)n2)c(-n2c3ccccc3c3ccccc32)c1 ; c1cc(-c2ccc3c(c2)oc2ccccc23)c(-c2nc(-n3c4ccccc4c4ccccc43)nc(-n3c4ccccc4c4ccccc43)n2)c(-n2c3ccccc3c3ccccc32)c1 ; c1cc(-c2cccc3c2oc2ccccc23)c(-c2nc(-n3c4ccccc4c4ccccc43)nc(-n3c4ccccc4c4ccccc43)n2)c(-n2c3ccccc3c3ccccc32)c1]

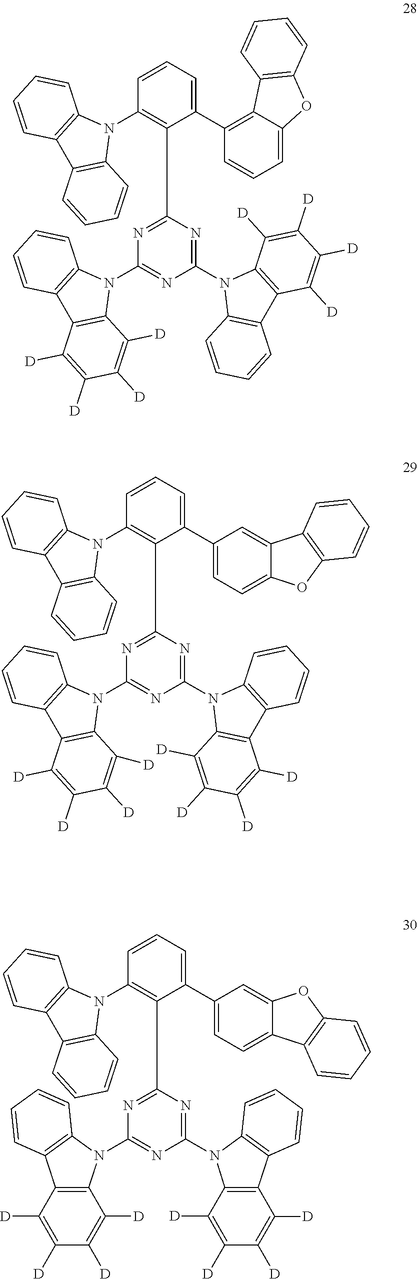 [SMILES: [2H]c1c([2H])c([2H])c2c(c1[2H])c1ccccc1n2-c1nc(-c2c(-c3cccc4oc5ccccc5c34)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2c(-c3ccc4oc5ccccc5c4c3)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2c(-c3ccc4c(c3)oc3ccccc34)cccc2-n2c3ccccc3c3ccccc32)nc(-n2c3ccccc3c3c([2H])c([2H])c([2H])c([2H])c32)n1]

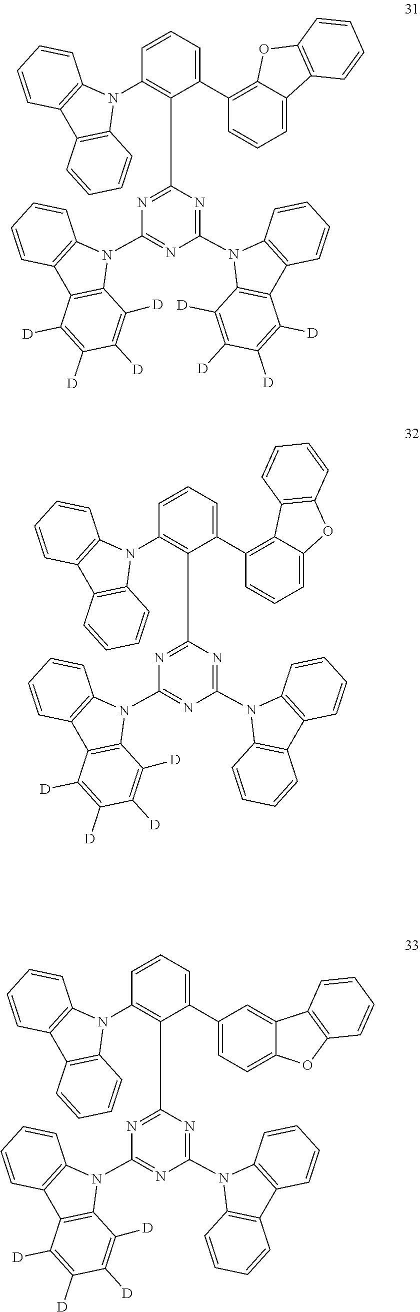 [SMILES: [2H]c1c([2H])c([2H])c2c(c1[2H])c1ccccc1n2-c1nc(-c2c(-c3cccc4c3oc3ccccc34)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2c(-c3cccc4oc5ccccc5c34)cccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2c(-c3ccc4oc5ccccc5c4c3)cccc2-n2c3ccccc3c3ccccc32)nc(-n2c3ccccc3c3ccccc32)n1]

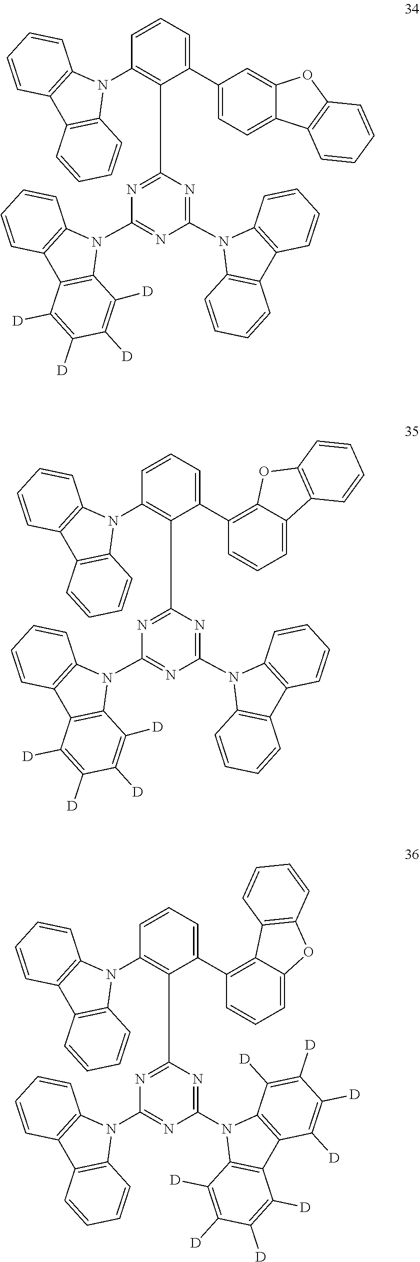 [SMILES: [2H]c1c([2H])c([2H])c2c(c1[2H])c1ccccc1n2-c1nc(-c2c(-c3ccc4c(c3)oc3ccccc34)cccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2c(-c3cccc4c3oc3ccccc34)cccc2-n2c3ccccc3c3ccccc32)nc(-n2c3ccccc3c3ccccc32)n1 ; [2H]c1c([2H])c([2H])c2c(c1[2H])c1c([2H])c([2H])c([2H])c([2H])c1n2-c1nc(-c2c(-c3cccc4oc5ccccc5c34)cccc2-n2c3ccccc3c3ccccc32)nc(-n2c3ccccc3c3ccccc32)n1]

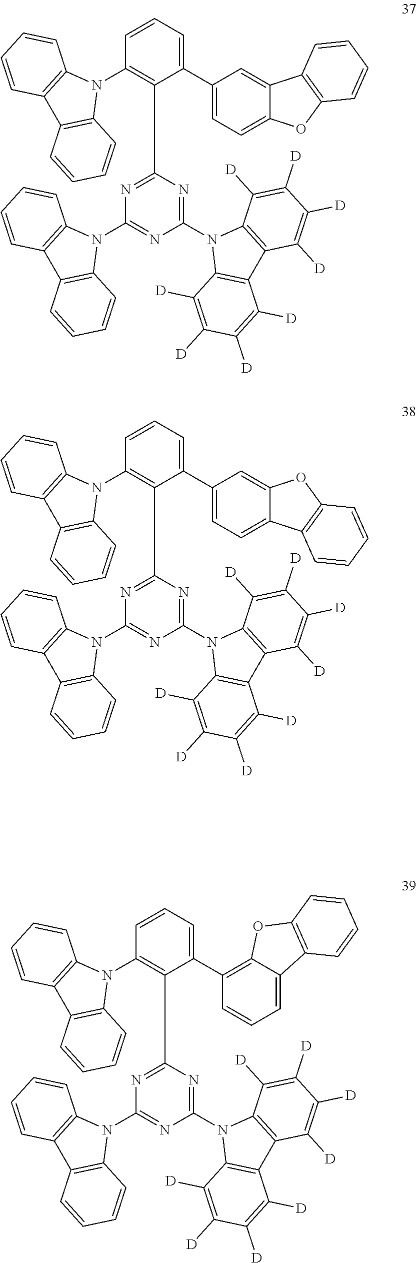 [SMILES: [2H]c1c([2H])c([2H])c2c(c1[2H])c1c([2H])c([2H])c([2H])c([2H])c1n2-c1nc(-c2c(-c3ccc4oc5ccccc5c4c3)cccc2-n2c3ccccc3c3ccccc32)nc(-n2c3ccccc3c3ccccc32)n1 ; [2H]c1c([2H])c([2H])c2c(c1[2H])c1c([2H])c([2H])c([2H])c([2H])c1n2-c1nc(-c2c(-c3ccc4c(c3)oc3ccccc34)cccc2-n2c3ccccc3c3ccccc32)nc(-n2c3ccccc3c3ccccc32)n1 ; [2H]c1c([2H])c([2H])c2c(c1[2H])c1c([2H])c([2H])c([2H])c([2H])c1n2-c1nc(-c2c(-c3cccc4c3oc3ccccc34)cccc2-n2c3ccccc3c3ccccc32)nc(-n2c3ccccc3c3ccccc32)n1]

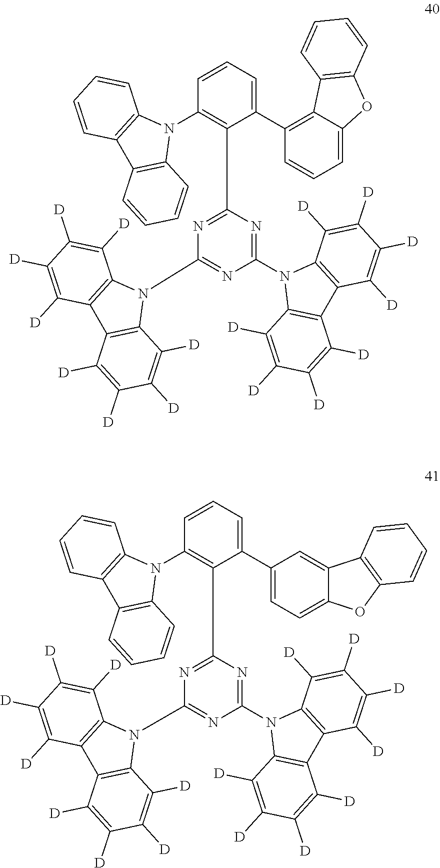 [SMILES: [2H]c1c([2H])c([2H])c2c(c1[2H])c1c([2H])c([2H])c([2H])c([2H])c1n2-c1nc(-c2c(-c3cccc4oc5ccccc5c34)cccc2-n2c3ccccc3c3ccccc32)nc(-n2c3c([2H])c([2H])c([2H])c([2H])c3c3c([2H])c([2H])c([2H])c([2H])c32)n1 ; [2H]c1c([2H])c([2H])c2c(c1[2H])c1c([2H])c([2H])c([2H])c([2H])c1n2-c1nc(-c2c(-c3ccc4oc5ccccc5c4c3)cccc2-n2c3ccccc3c3ccccc32)nc(-n2c3c([2H])c([2H])c([2H])c([2H])c3c3c([2H])c([2H])c([2H])c([2H])c32)n1]

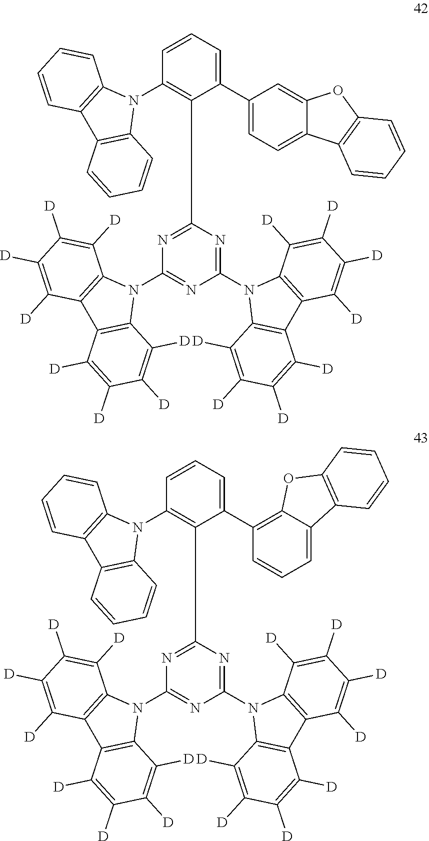 [SMILES: [2H]c1c([2H])c([2H])c2c(c1[2H])c1c([2H])c([2H])c([2H])c([2H])c1n2-c1nc(-c2c(-c3ccc4c(c3)oc3ccccc34)cccc2-n2c3ccccc3c3ccccc32)nc(-n2c3c([2H])c([2H])c([2H])c([2H])c3c3c([2H])c([2H])c([2H])c([2H])c32)n1 ; [2H]c1c([2H])c([2H])c2c(c1[2H])c1c([2H])c([2H])c([2H])c([2H])c1n2-c1nc(-c2c(-c3cccc4c3oc3ccccc34)cccc2-n2c3ccccc3c3ccccc32)nc(-n2c3c([2H])c([2H])c([2H])c([2H])c3c3c([2H])c([2H])c([2H])c([2H])c32)n1]

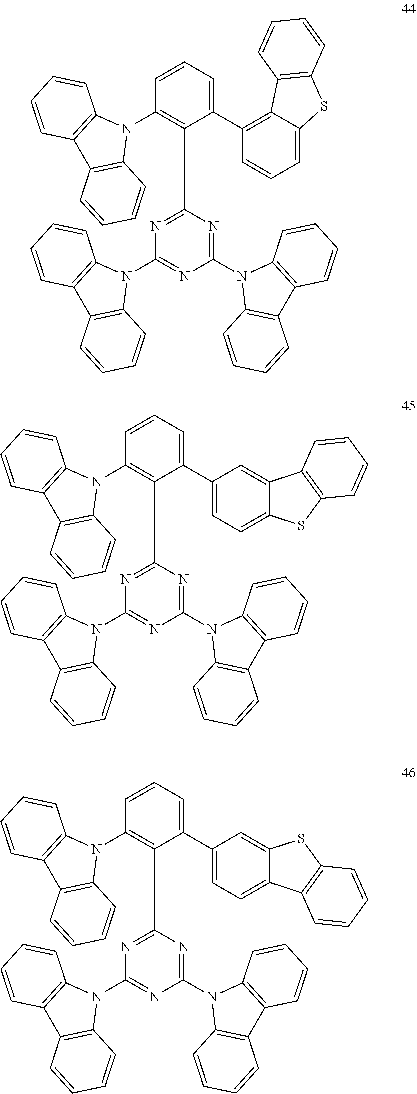 [SMILES: c1cc(-c2cccc3sc4ccccc4c23)c(-c2nc(-n3c4ccccc4c4ccccc43)nc(-n3c4ccccc4c4ccccc43)n2)c(-n2c3ccccc3c3ccccc32)c1 ; c1cc(-c2ccc3sc4ccccc4c3c2)c(-c2nc(-n3c4ccccc4c4ccccc43)nc(-n3c4ccccc4c4ccccc43)n2)c(-n2c3ccccc3c3ccccc32)c1 ; c1cc(-c2ccc3c(c2)sc2ccccc23)c(-c2nc(-n3c4ccccc4c4ccccc43)nc(-n3c4ccccc4c4ccccc43)n2)c(-n2c3ccccc3c3ccccc32)c1]

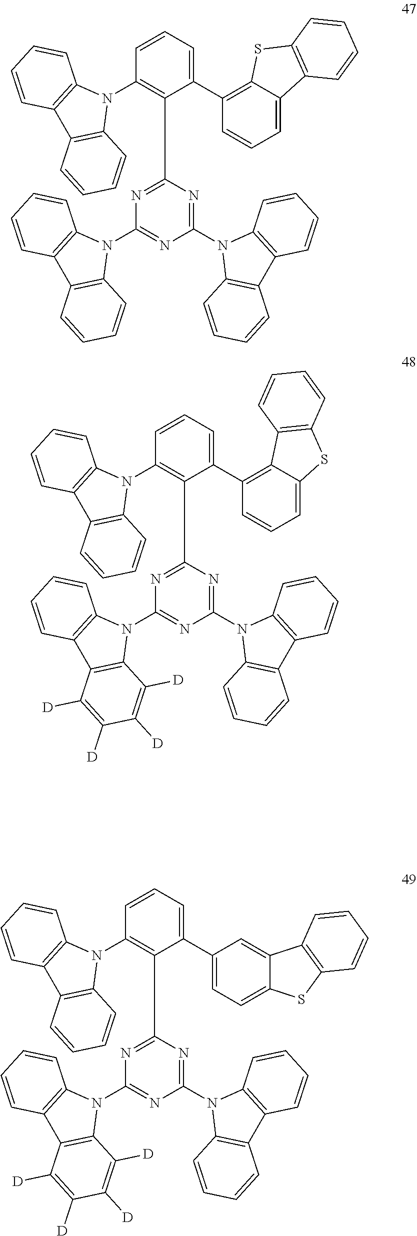

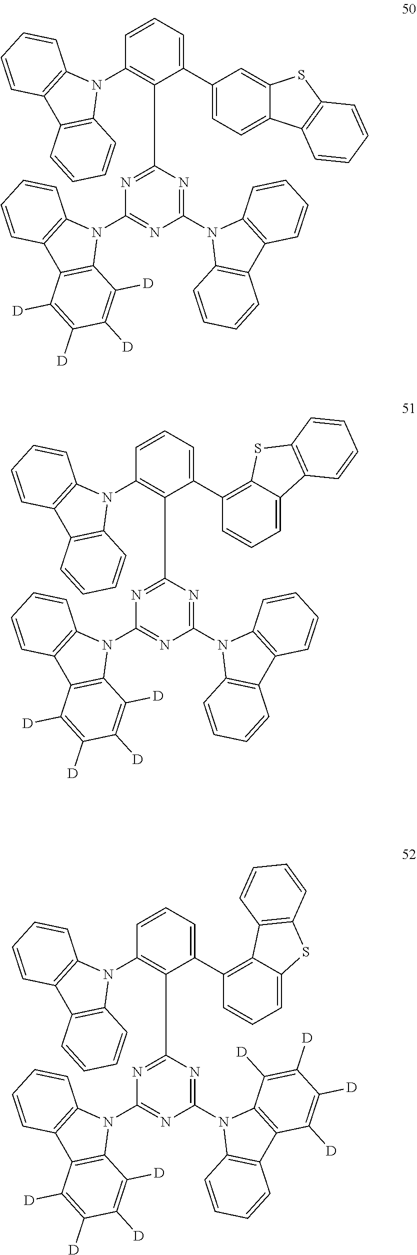 [SMILES: [2H]c1c([2H])c([2H])c2c(c1[2H])c1ccccc1n2-c1nc(-c2c(-c3ccc4c(c3)sc3ccccc34)cccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2c(-c3cccc4c3sc3ccccc34)cccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2c(-c3cccc4sc5ccccc5c34)cccc2-n2c3ccccc3c3ccccc32)nc(-n2c3ccccc3c3c([2H])c([2H])c([2H])c([2H])c32)n1]

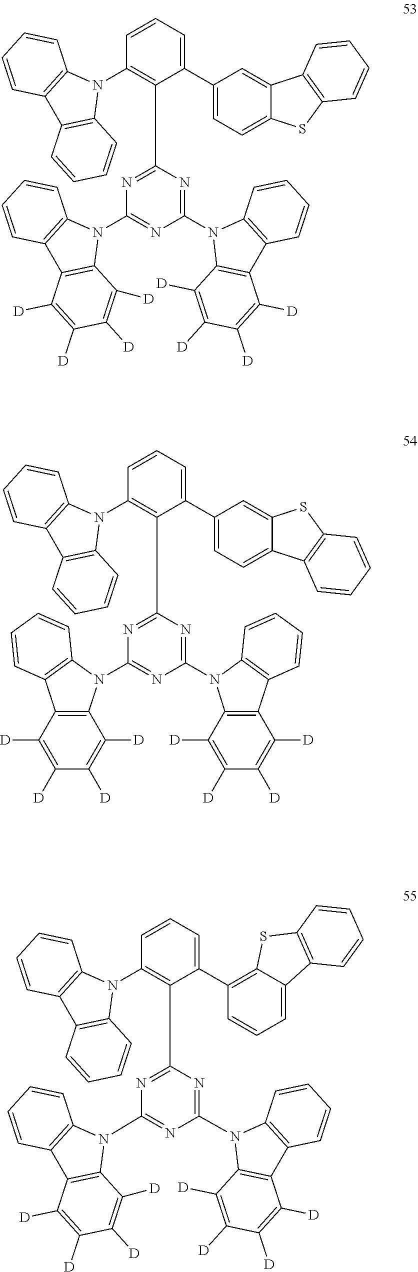 [SMILES: [2H]c1c([2H])c([2H])c2c(c1[2H])c1ccccc1n2-c1nc(-c2c(-c3ccc4sc5ccccc5c4c3)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2c(-c3ccc4c(c3)sc3ccccc34)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2c(-c3cccc4c3sc3ccccc34)cccc2-n2c3ccccc3c3ccccc32)nc(-n2c3ccccc3c3c([2H])c([2H])c([2H])c([2H])c32)n1]

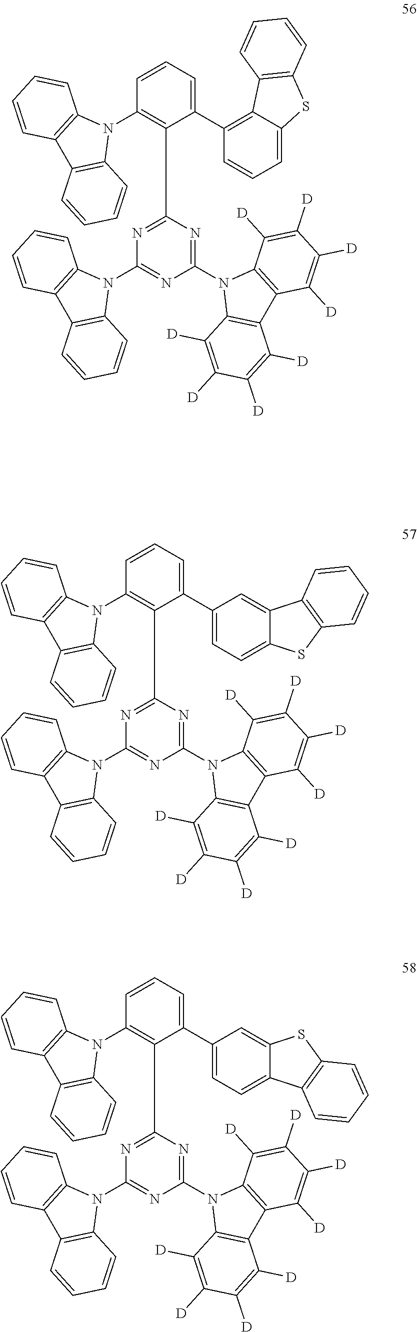 [SMILES: [2H]c1c([2H])c([2H])c2c(c1[2H])c1c([2H])c([2H])c([2H])c([2H])c1n2-c1nc(-c2c(-c3cccc4sc5ccccc5c34)cccc2-n2c3ccccc3c3ccccc32)nc(-n2c3ccccc3c3ccccc32)n1 ; [2H]c1c([2H])c([2H])c2c(c1[2H])c1c([2H])c([2H])c([2H])c([2H])c1n2-c1nc(-c2c(-c3ccc4sc5ccccc5c4c3)cccc2-n2c3ccccc3c3ccccc32)nc(-n2c3ccccc3c3ccccc32)n1 ; [2H]c1c([2H])c([2H])c2c(c1[2H])c1c([2H])c([2H])c([2H])c([2H])c1n2-c1nc(-c2c(-c3ccc4c(c3)sc3ccccc34)cccc2-n2c3ccccc3c3ccccc32)nc(-n2c3ccccc3c3ccccc32)n1]

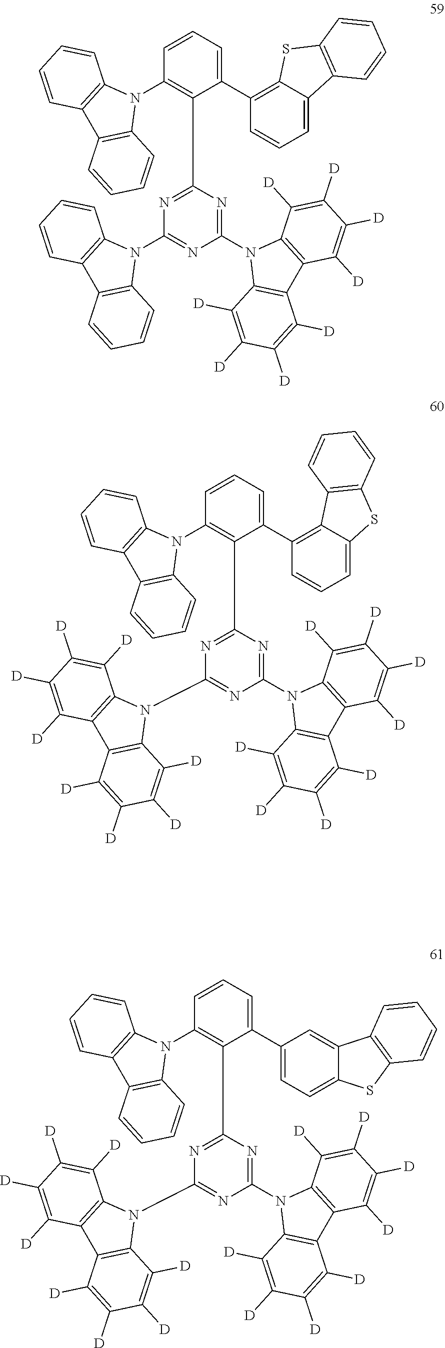

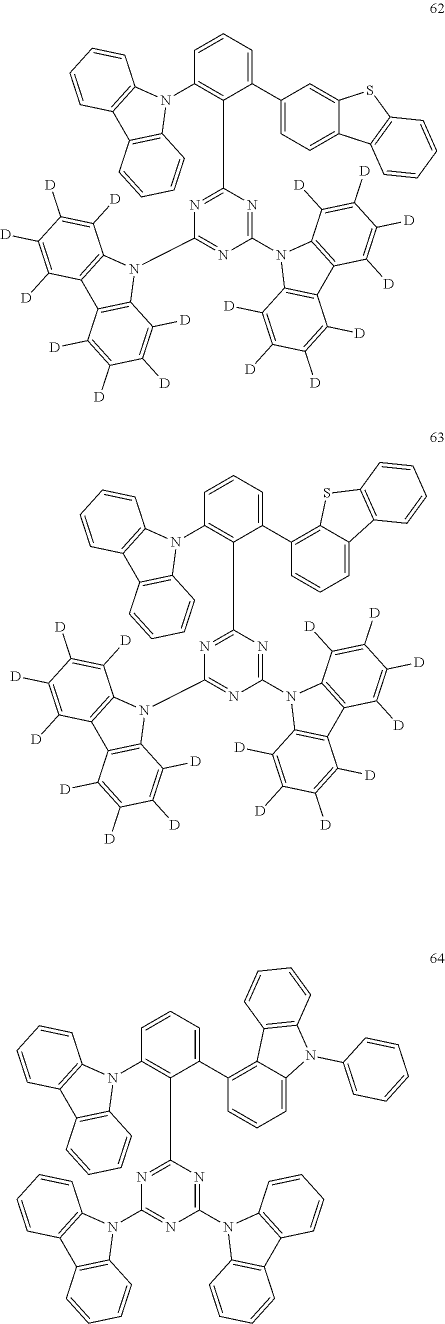

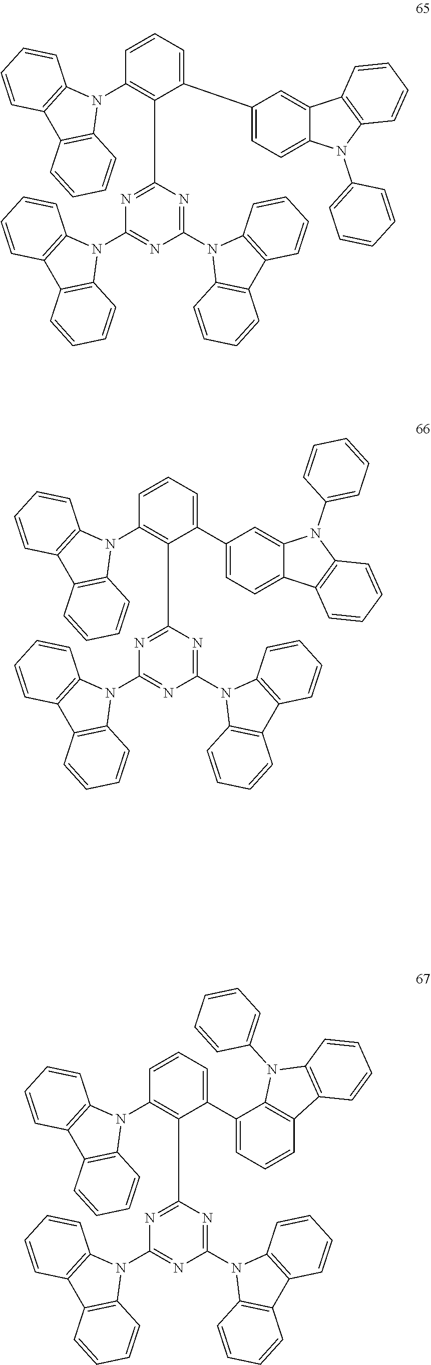 [SMILES: c1ccc(-n2c3ccccc3c3cc(-c4cccc(-n5c6ccccc6c6ccccc65)c4-c4nc(-n5c6ccccc6c6ccccc65)nc(-n5c6ccccc6c6ccccc65)n4)ccc32)cc1 ; c1ccc(-n2c3ccccc3c3ccc(-c4cccc(-n5c6ccccc6c6ccccc65)c4-c4nc(-n5c6ccccc6c6ccccc65)nc(-n5c6ccccc6c6ccccc65)n4)cc32)cc1 ; c1ccc(-n2c3ccccc3c3cccc(-c4cccc(-n5c6ccccc6c6ccccc65)c4-c4nc(-n5c6ccccc6c6ccccc65)nc(-n5c6ccccc6c6ccccc65)n4)c32)cc1]

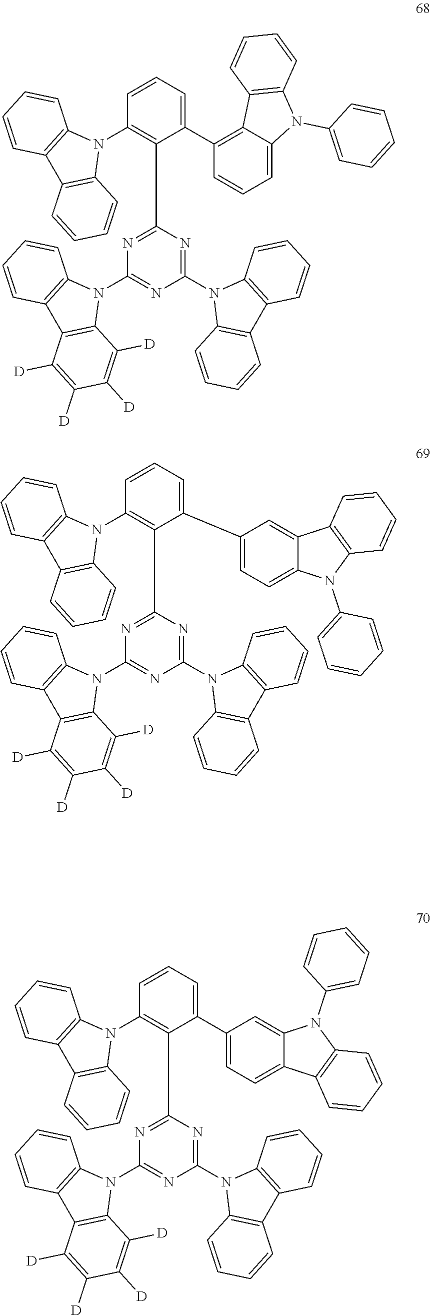 [SMILES: [2H]c1c([2H])c([2H])c2c(c1[2H])c1ccccc1n2-c1nc(-c2c(-c3cccc4c3c3ccccc3n4-c3ccccc3)cccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2c(-c3ccc4c(c3)c3ccccc3n4-c3ccccc3)cccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2c(-c3ccc4c5ccccc5n(-c5ccccc5)c4c3)cccc2-n2c3ccccc3c3ccccc32)nc(-n2c3ccccc3c3ccccc32)n1]

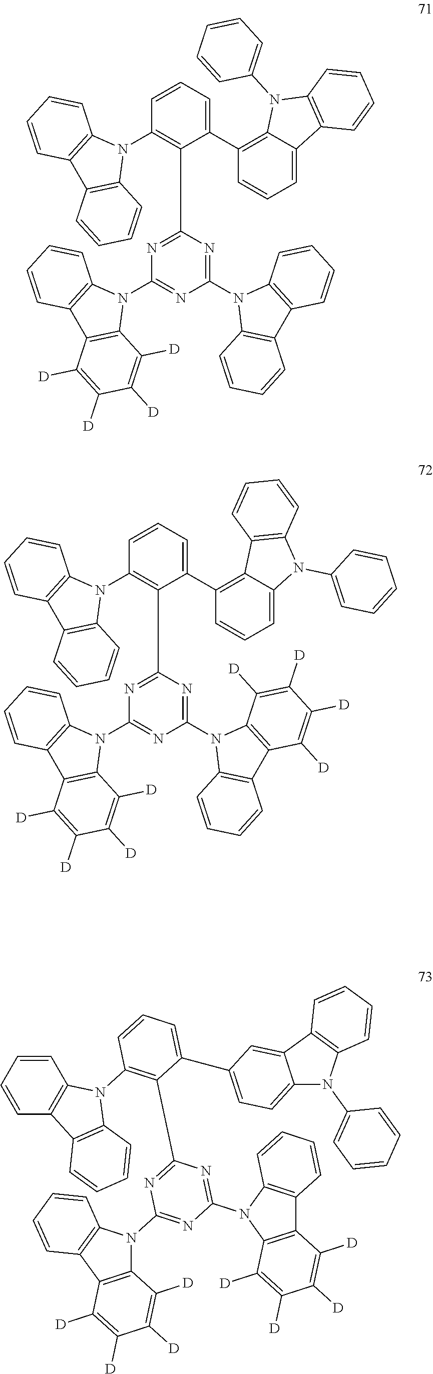 [SMILES: [2H]c1c([2H])c([2H])c2c(c1[2H])c1ccccc1n2-c1nc(-c2c(-c3cccc4c5ccccc5n(-c5ccccc5)c34)cccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2c(-c3cccc4c3c3ccccc3n4-c3ccccc3)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2c(-c3ccc4c(c3)c3ccccc3n4-c3ccccc3)cccc2-n2c3ccccc3c3ccccc32)nc(-n2c3ccccc3c3c([2H])c([2H])c([2H])c([2H])c32)n1]

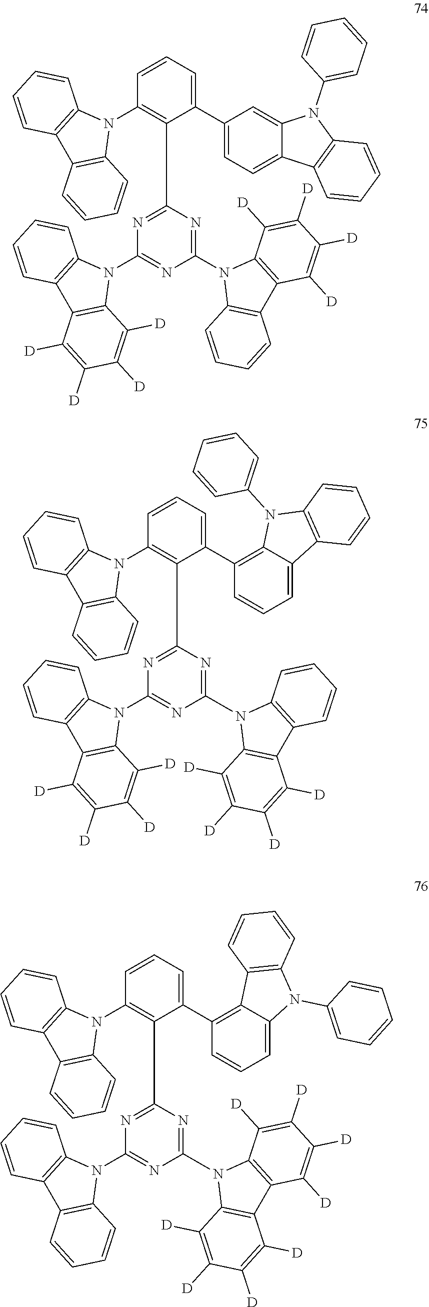 [SMILES: [2H]c1c([2H])c([2H])c2c(c1[2H])c1ccccc1n2-c1nc(-c2c(-c3ccc4c5ccccc5n(-c5ccccc5)c4c3)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2c(-c3cccc4c5ccccc5n(-c5ccccc5)c34)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1c([2H])c([2H])c([2H])c([2H])c1n2-c1nc(-c2c(-c3cccc4c3c3ccccc3n4-c3ccccc3)cccc2-n2c3ccccc3c3ccccc32)nc(-n2c3ccccc3c3ccccc32)n1]

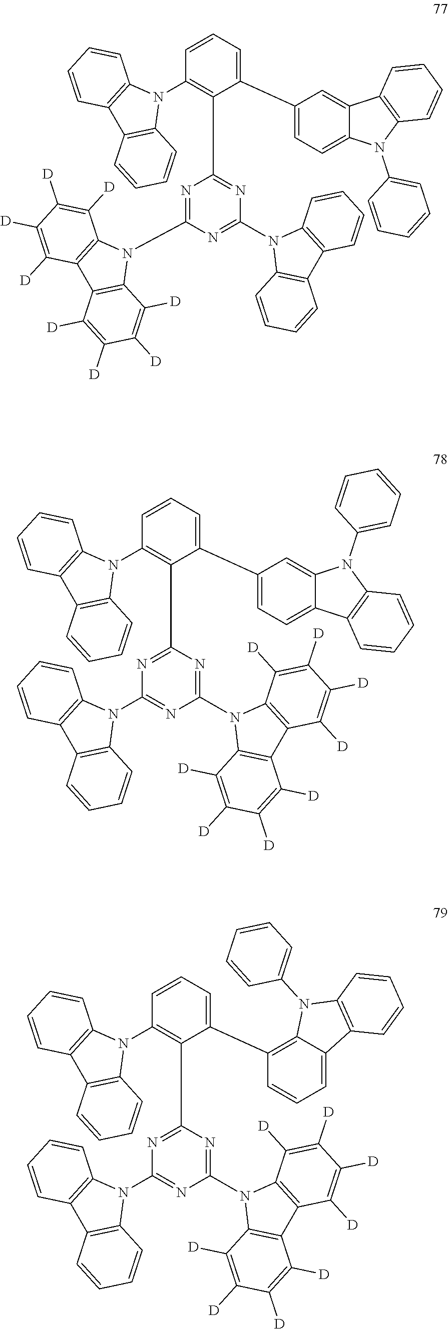 [SMILES: [2H]c1c([2H])c([2H])c2c(c1[2H])c1c([2H])c([2H])c([2H])c([2H])c1n2-c1nc(-c2c(-c3ccc4c(c3)c3ccccc3n4-c3ccccc3)cccc2-n2c3ccccc3c3ccccc32)nc(-n2c3ccccc3c3ccccc32)n1 ; [2H]c1c([2H])c([2H])c2c(c1[2H])c1c([2H])c([2H])c([2H])c([2H])c1n2-c1nc(-c2c(-c3ccc4c5ccccc5n(-c5ccccc5)c4c3)cccc2-n2c3ccccc3c3ccccc32)nc(-n2c3ccccc3c3ccccc32)n1 ; [2H]c1c([2H])c([2H])c2c(c1[2H])c1c([2H])c([2H])c([2H])c([2H])c1n2-c1nc(-c2c(-c3cccc4c5ccccc5n(-c5ccccc5)c34)cccc2-n2c3ccccc3c3ccccc32)nc(-n2c3ccccc3c3ccccc32)n1]

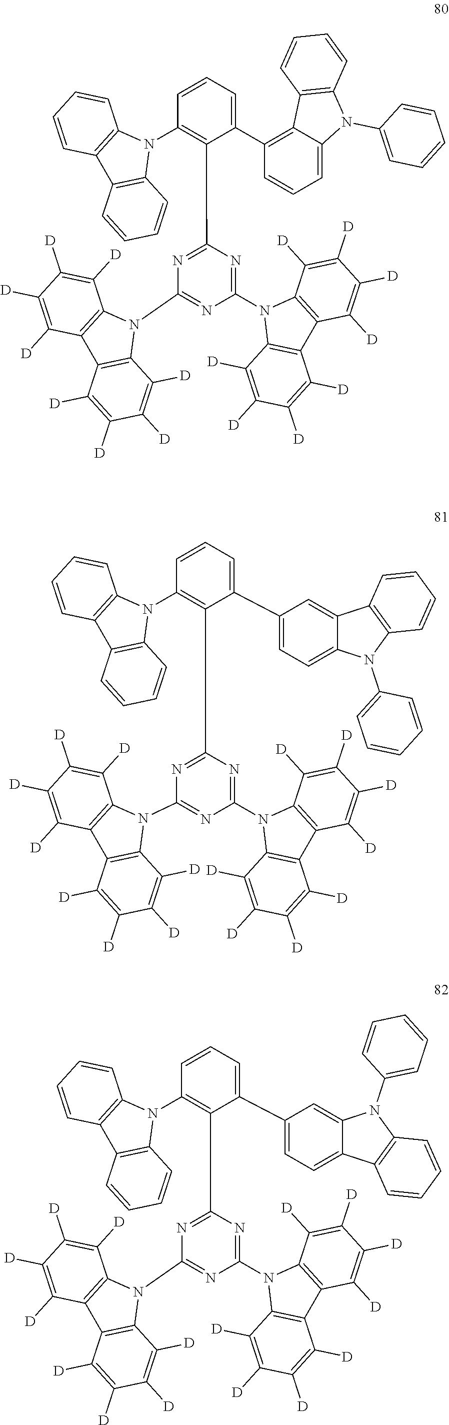

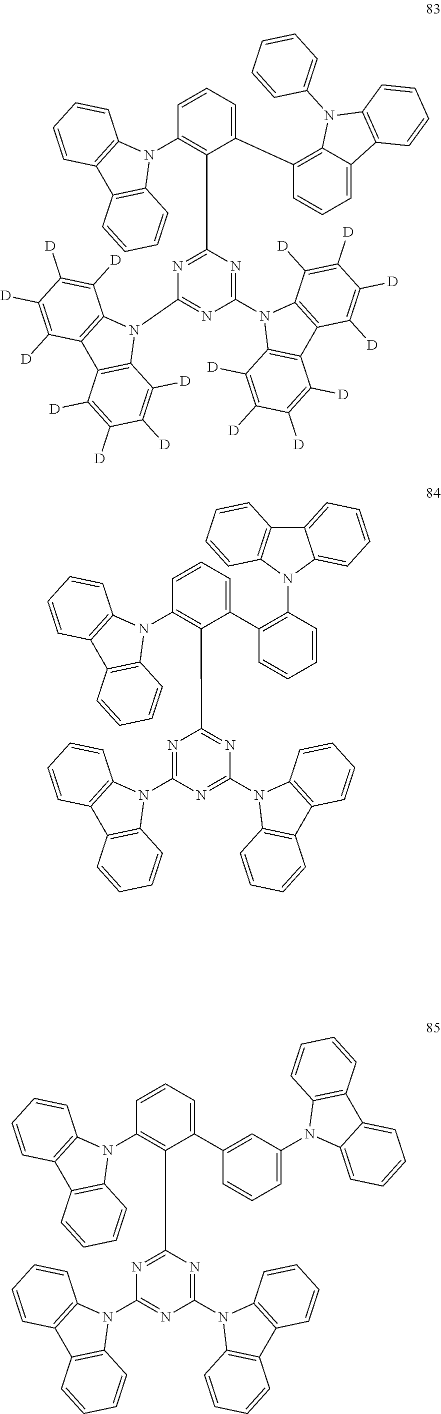 [SMILES: [2H]c1c([2H])c([2H])c2c(c1[2H])c1c([2H])c([2H])c([2H])c([2H])c1n2-c1nc(-c2c(-c3cccc4c5ccccc5n(-c5ccccc5)c34)cccc2-n2c3ccccc3c3ccccc32)nc(-n2c3c([2H])c([2H])c([2H])c([2H])c3c3c([2H])c([2H])c([2H])c([2H])c32)n1 ; c1ccc(-n2c3ccccc3c3ccccc32)c(-c2cccc(-n3c4ccccc4c4ccccc43)c2-c2nc(-n3c4ccccc4c4ccccc43)nc(-n3c4ccccc4c4ccccc43)n2)c1 ; c1cc(-c2cccc(-n3c4ccccc4c4ccccc43)c2-c2nc(-n3c4ccccc4c4ccccc43)nc(-n3c4ccccc4c4ccccc43)n2)cc(-n2c3ccccc3c3ccccc32)c1]

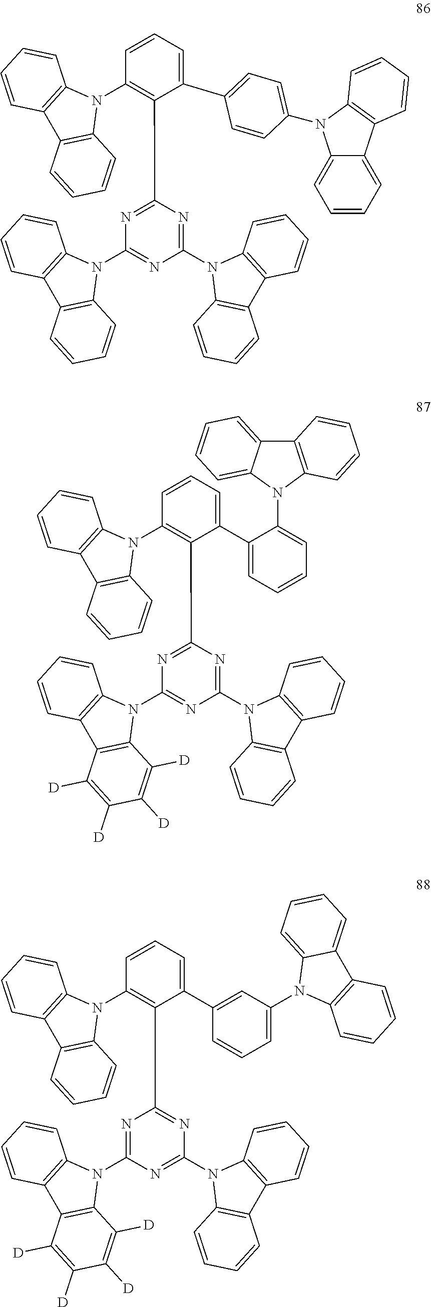 [SMILES: c1cc(-c2ccc(-n3c4ccccc4c4ccccc43)cc2)c(-c2nc(-n3c4ccccc4c4ccccc43)nc(-n3c4ccccc4c4ccccc43)n2)c(-n2c3ccccc3c3ccccc32)c1 ; [2H]c1c([2H])c([2H])c2c(c1[2H])c1ccccc1n2-c1nc(-c2c(-c3ccccc3-n3c4ccccc4c4ccccc43)cccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2c(-c3cccc(-n4c5ccccc5c5ccccc54)c3)cccc2-n2c3ccccc3c3ccccc32)nc(-n2c3ccccc3c3ccccc32)n1]

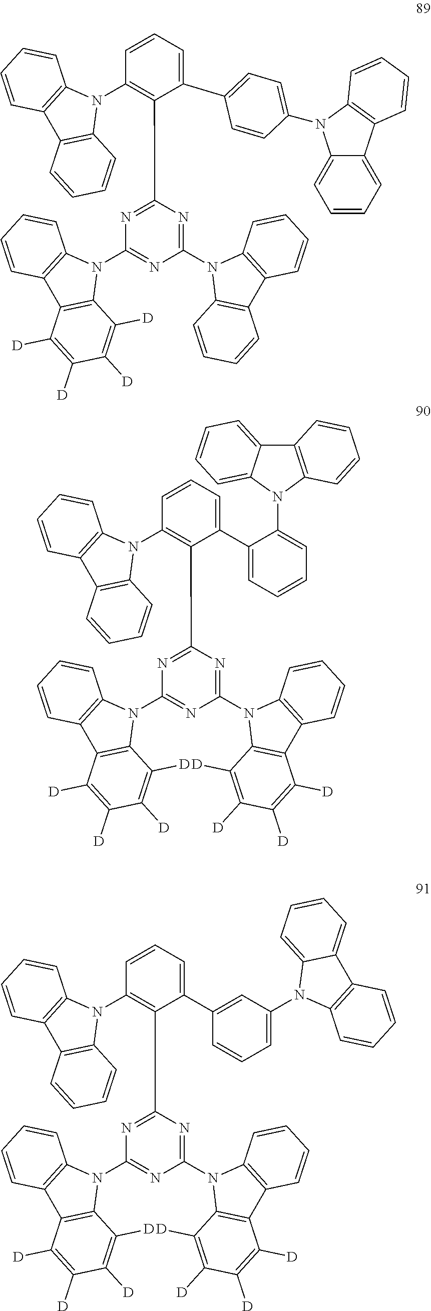 [SMILES: [2H]c1c([2H])c([2H])c2c(c1[2H])c1ccccc1n2-c1nc(-c2c(-c3ccc(-n4c5ccccc5c5ccccc54)cc3)cccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2c(-c3ccccc3-n3c4ccccc4c4ccccc43)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2c(-c3cccc(-n4c5ccccc5c5ccccc54)c3)cccc2-n2c3ccccc3c3ccccc32)nc(-n2c3ccccc3c3c([2H])c([2H])c([2H])c([2H])c32)n1]

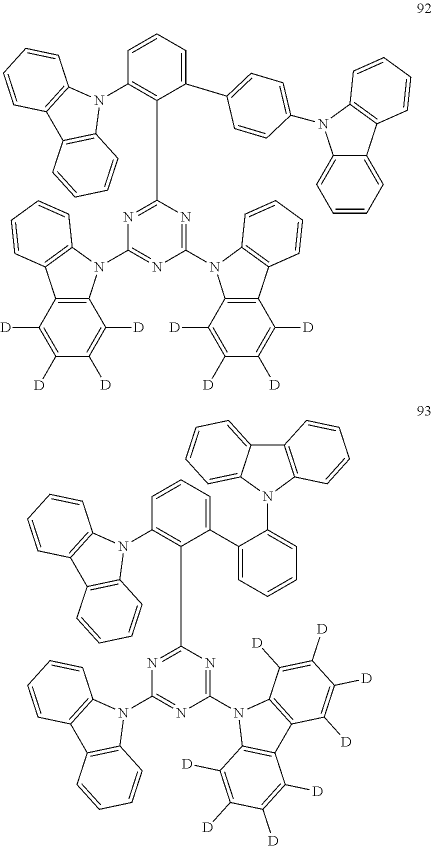

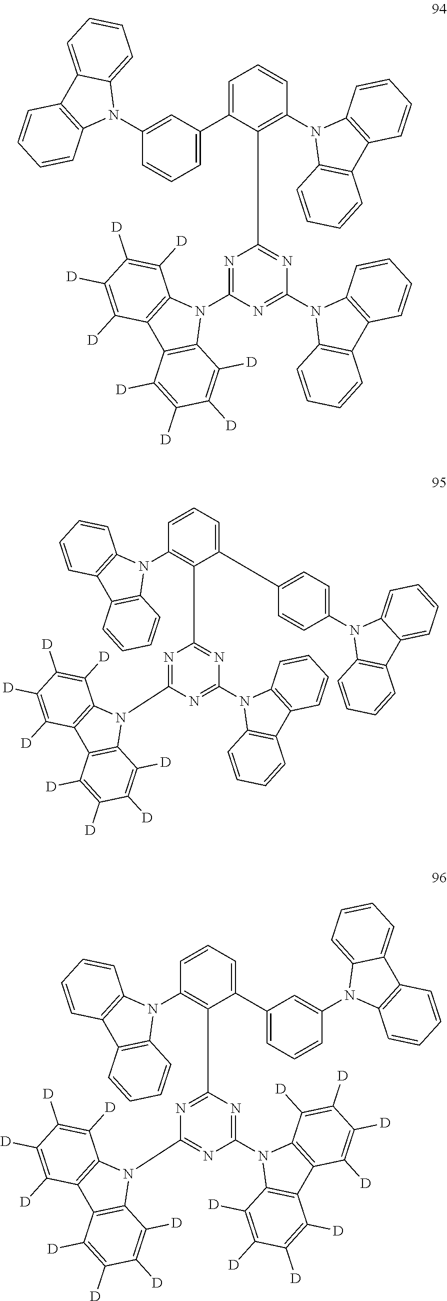

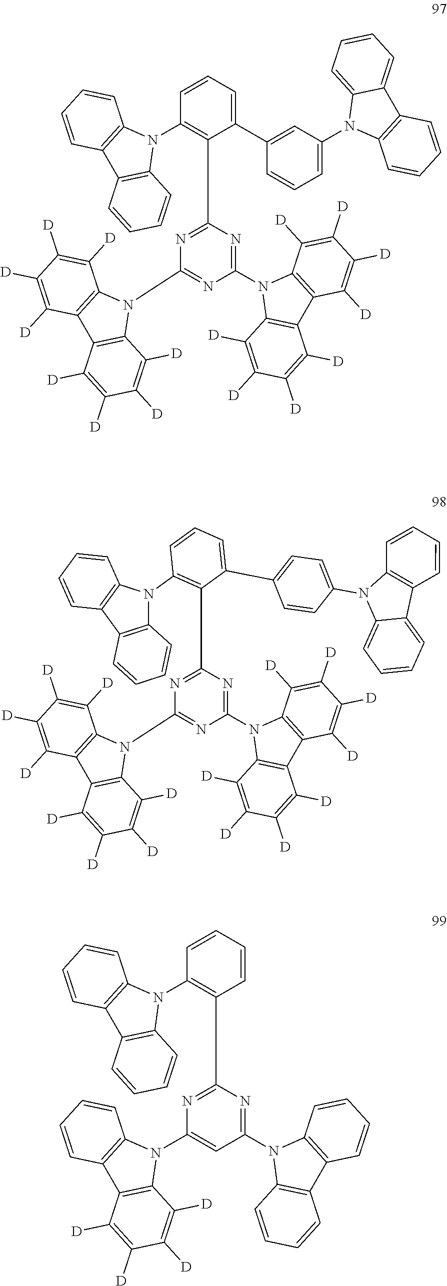

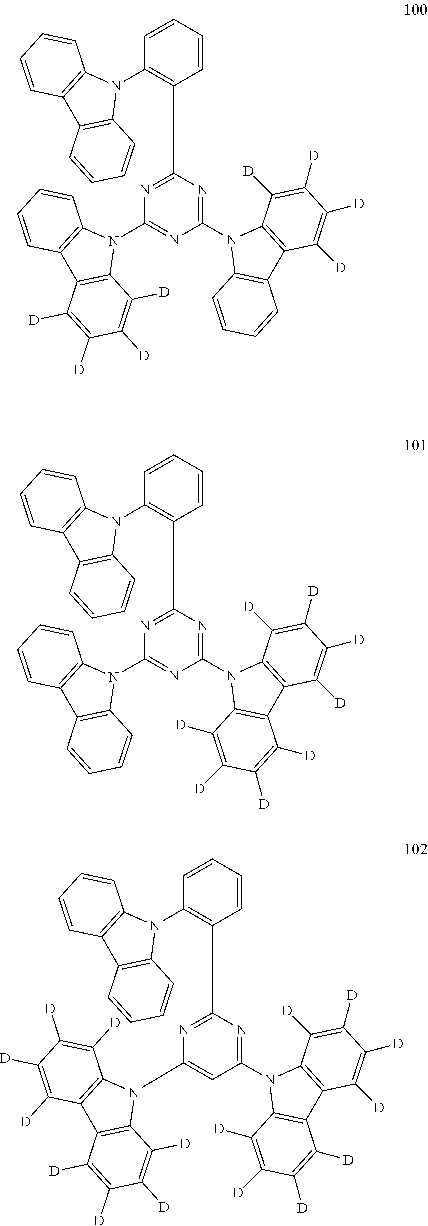 [SMILES: [2H]c1c([2H])c([2H])c2c(c1[2H])c1ccccc1n2-c1nc(-c2ccccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1c([2H])c([2H])c([2H])c([2H])c1n2-c1nc(-c2ccccc2-n2c3ccccc3c3ccccc32)nc(-n2c3ccccc3c3ccccc32)n1 ; [2H]c1c([2H])c([2H])c2c(c1[2H])c1c([2H])c([2H])c([2H])c([2H])c1n2-c1cc(-n2c3c([2H])c([2H])c([2H])c([2H])c3c3c([2H])c([2H])c([2H])c([2H])c32)nc(-c2ccccc2-n2c3ccccc3c3ccccc32)n1]

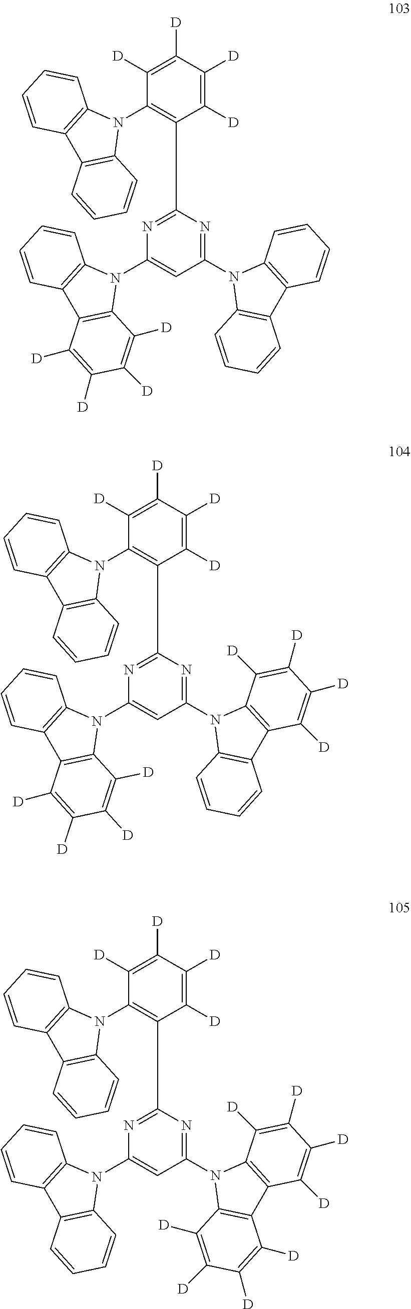 [SMILES: [2H]c1c([2H])c([2H])c(-n2c3ccccc3c3ccccc32)c(-c2nc(-n3c4ccccc4c4ccccc43)cc(-n3c4ccccc4c4c([2H])c([2H])c([2H])c([2H])c43)n2)c1[2H] ; [2H]c1c([2H])c([2H])c(-n2c3ccccc3c3ccccc32)c(-c2nc(-n3c4ccccc4c4c([2H])c([2H])c([2H])c([2H])c43)cc(-n3c4ccccc4c4c([2H])c([2H])c([2H])c([2H])c43)n2)c1[2H] ; [2H]c1c([2H])c([2H])c(-n2c3ccccc3c3ccccc32)c(-c2nc(-n3c4ccccc4c4ccccc43)cc(-n3c4c([2H])c([2H])c([2H])c([2H])c4c4c([2H])c([2H])c([2H])c([2H])c43)n2)c1[2H]]

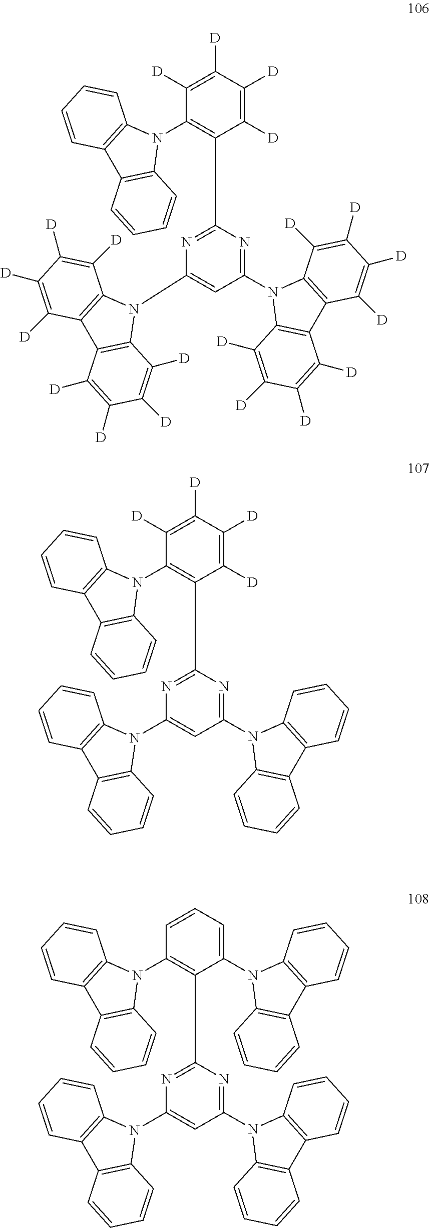 [SMILES: [2H]c1c([2H])c([2H])c(-n2c3ccccc3c3ccccc32)c(-c2nc(-n3c4c([2H])c([2H])c([2H])c([2H])c4c4c([2H])c([2H])c([2H])c([2H])c43)cc(-n3c4c([2H])c([2H])c([2H])c([2H])c4c4c([2H])c([2H])c([2H])c([2H])c43)n2)c1[2H] ; [2H]c1c([2H])c([2H])c(-n2c3ccccc3c3ccccc32)c(-c2nc(-n3c4ccccc4c4ccccc43)cc(-n3c4ccccc4c4ccccc43)n2)c1[2H] ; c1cc(-n2c3ccccc3c3ccccc32)c(-c2nc(-n3c4ccccc4c4ccccc43)cc(-n3c4ccccc4c4ccccc43)n2)c(-n2c3ccccc3c3ccccc32)c1]

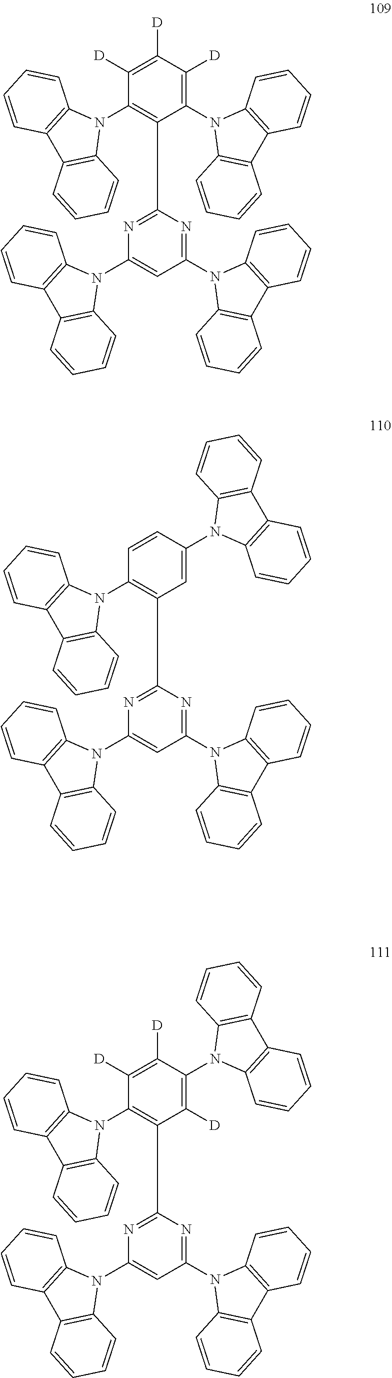 [SMILES: [2H]c1c([2H])c(-n2c3ccccc3c3ccccc32)c(-c2nc(-n3c4ccccc4c4ccccc43)cc(-n3c4ccccc4c4ccccc43)n2)c(-n2c3ccccc3c3ccccc32)c1[2H] ; c1ccc2c(c1)c1ccccc1n2-c1ccc(-n2c3ccccc3c3ccccc32)c(-c2nc(-n3c4ccccc4c4ccccc43)cc(-n3c4ccccc4c4ccccc43)n2)c1 ; [2H]c1c([2H])c(-n2c3ccccc3c3ccccc32)c(-c2nc(-n3c4ccccc4c4ccccc43)cc(-n3c4ccccc4c4ccccc43)n2)c([2H])c1-n1c2ccccc2c2ccccc21]

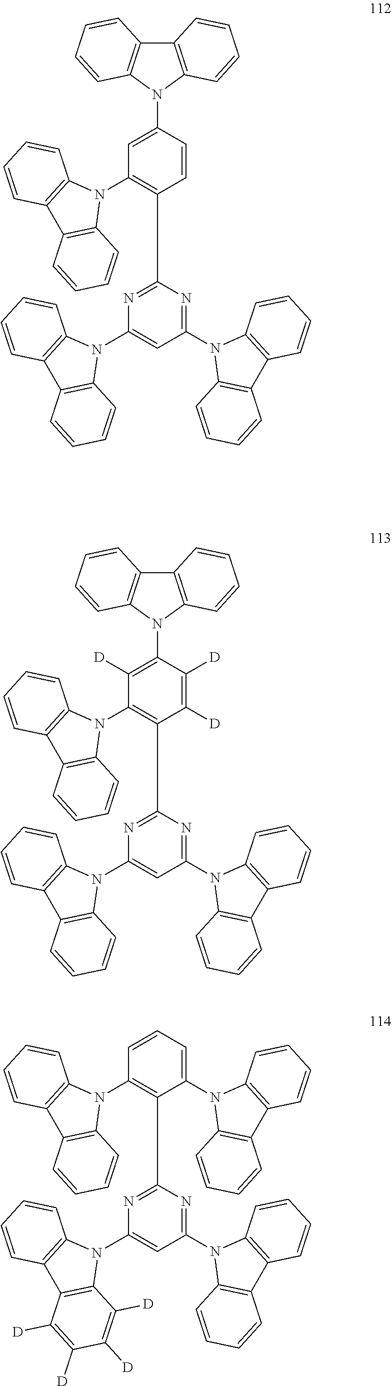 [SMILES: c1ccc2c(c1)c1ccccc1n2-c1ccc(-c2nc(-n3c4ccccc4c4ccccc43)cc(-n3c4ccccc4c4ccccc43)n2)c(-n2c3ccccc3c3ccccc32)c1 ; [2H]c1c([2H])c(-n2c3ccccc3c3ccccc32)c([2H])c(-n2c3ccccc3c3ccccc32)c1-c1nc(-n2c3ccccc3c3ccccc32)cc(-n2c3ccccc3c3ccccc32)n1 ; [2H]c1c([2H])c([2H])c2c(c1[2H])c1ccccc1n2-c1cc(-n2c3ccccc3c3ccccc32)nc(-c2c(-n3c4ccccc4c4ccccc43)cccc2-n2c3ccccc3c3ccccc32)n1]

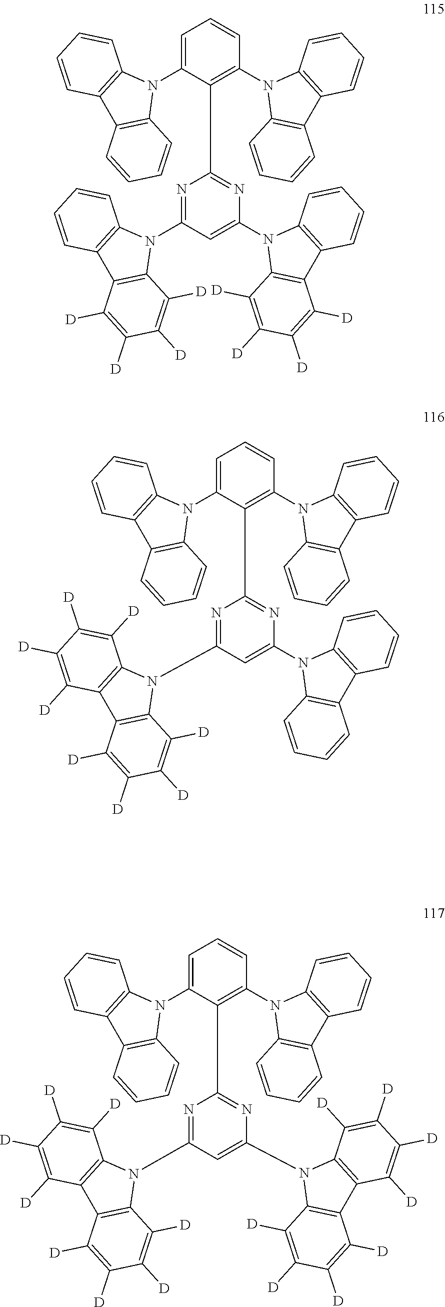

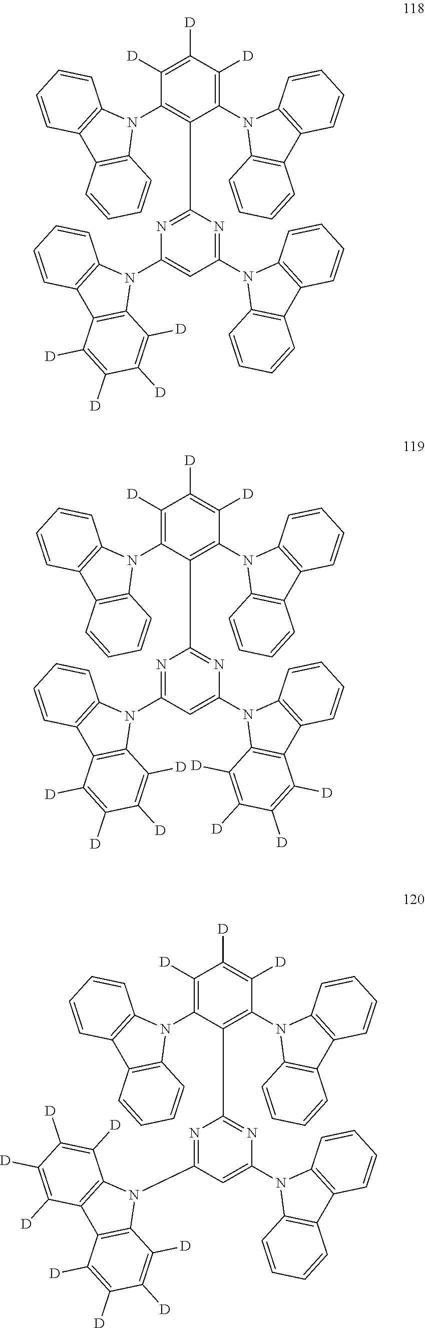 [SMILES: [2H]c1c([2H])c(-n2c3ccccc3c3ccccc32)c(-c2nc(-n3c4ccccc4c4ccccc43)cc(-n3c4ccccc4c4c([2H])c([2H])c([2H])c([2H])c43)n2)c(-n2c3ccccc3c3ccccc32)c1[2H] ; [2H]c1c([2H])c(-n2c3ccccc3c3ccccc32)c(-c2nc(-n3c4ccccc4c4c([2H])c([2H])c([2H])c([2H])c43)cc(-n3c4ccccc4c4c([2H])c([2H])c([2H])c([2H])c43)n2)c(-n2c3ccccc3c3ccccc32)c1[2H] ; [2H]c1c([2H])c(-n2c3ccccc3c3ccccc32)c(-c2nc(-n3c4ccccc4c4ccccc43)cc(-n3c4c([2H])c([2H])c([2H])c([2H])c4c4c([2H])c([2H])c([2H])c([2H])c43)n2)c(-n2c3ccccc3c3ccccc32)c1[2H]]

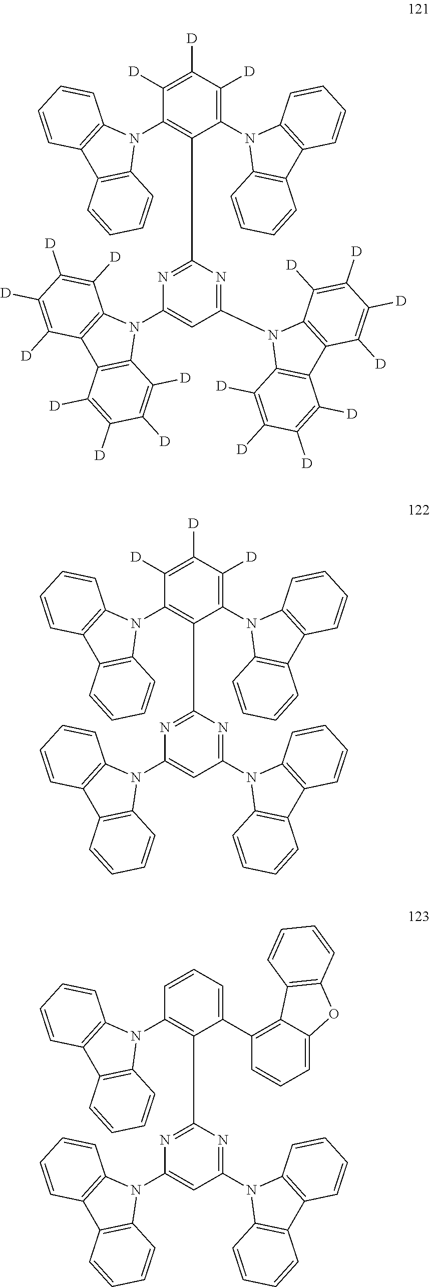 [SMILES: [2H]c1c([2H])c(-n2c3ccccc3c3ccccc32)c(-c2nc(-n3c4c([2H])c([2H])c([2H])c([2H])c4c4c([2H])c([2H])c([2H])c([2H])c43)cc(-n3c4c([2H])c([2H])c([2H])c([2H])c4c4c([2H])c([2H])c([2H])c([2H])c43)n2)c(-n2c3ccccc3c3ccccc32)c1[2H] ; [2H]c1c([2H])c(-n2c3ccccc3c3ccccc32)c(-c2nc(-n3c4ccccc4c4ccccc43)cc(-n3c4ccccc4c4ccccc43)n2)c(-n2c3ccccc3c3ccccc32)c1[2H] ; c1cc(-c2cccc3oc4ccccc4c23)c(-c2nc(-n3c4ccccc4c4ccccc43)cc(-n3c4ccccc4c4ccccc43)n2)c(-n2c3ccccc3c3ccccc32)c1]

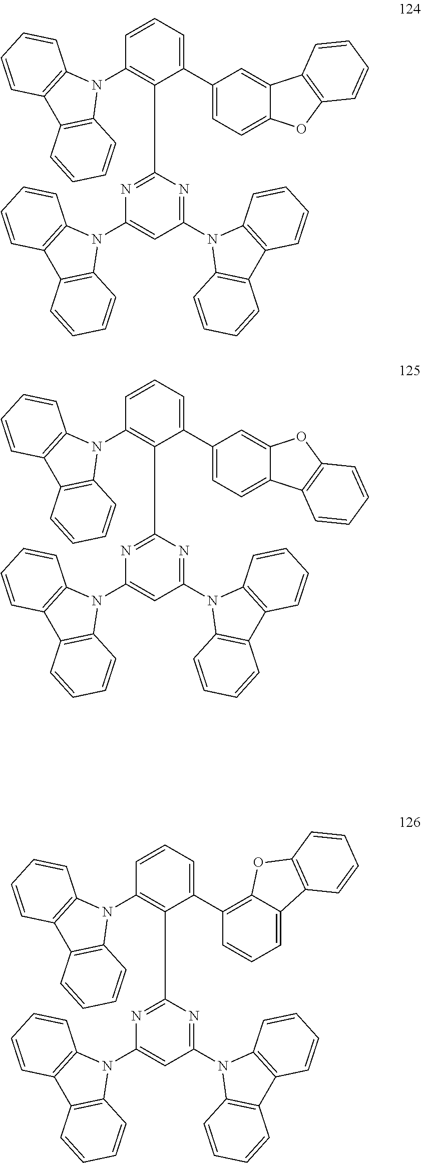 [SMILES: c1cc(-c2ccc3oc4ccccc4c3c2)c(-c2nc(-n3c4ccccc4c4ccccc43)cc(-n3c4ccccc4c4ccccc43)n2)c(-n2c3ccccc3c3ccccc32)c1 ; c1cc(-c2ccc3c(c2)oc2ccccc23)c(-c2nc(-n3c4ccccc4c4ccccc43)cc(-n3c4ccccc4c4ccccc43)n2)c(-n2c3ccccc3c3ccccc32)c1 ; c1cc(-c2cccc3c2oc2ccccc23)c(-c2nc(-n3c4ccccc4c4ccccc43)cc(-n3c4ccccc4c4ccccc43)n2)c(-n2c3ccccc3c3ccccc32)c1]

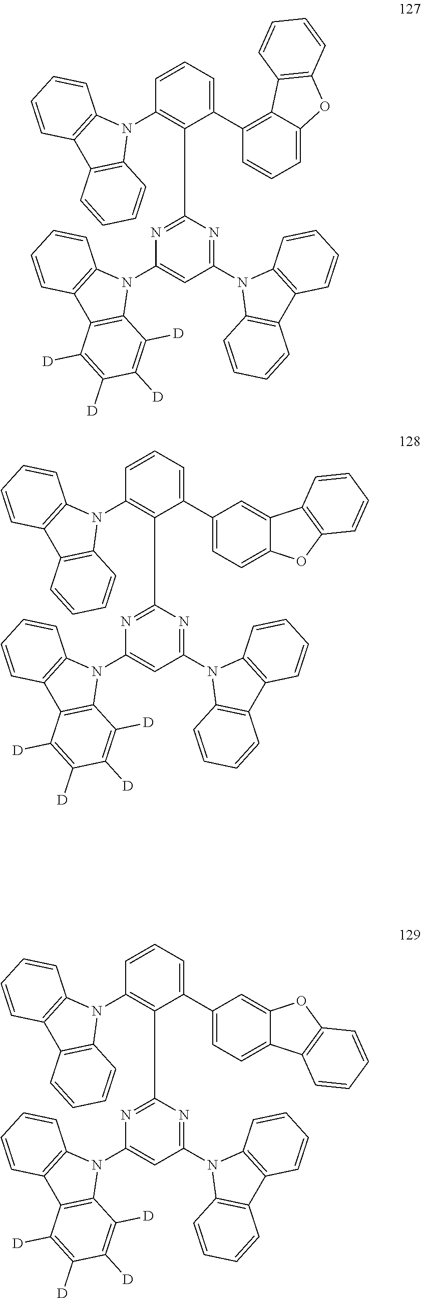 [SMILES: [2H]c1c([2H])c([2H])c2c(c1[2H])c1ccccc1n2-c1cc(-n2c3ccccc3c3ccccc32)nc(-c2c(-c3cccc4oc5ccccc5c34)cccc2-n2c3ccccc3c3ccccc32)n1 ; [2H]c1c([2H])c([2H])c2c(c1[2H])c1ccccc1n2-c1cc(-n2c3ccccc3c3ccccc32)nc(-c2c(-c3ccc4oc5ccccc5c4c3)cccc2-n2c3ccccc3c3ccccc32)n1 ; [2H]c1c([2H])c([2H])c2c(c1[2H])c1ccccc1n2-c1cc(-n2c3ccccc3c3ccccc32)nc(-c2c(-c3ccc4c(c3)oc3ccccc34)cccc2-n2c3ccccc3c3ccccc32)n1]

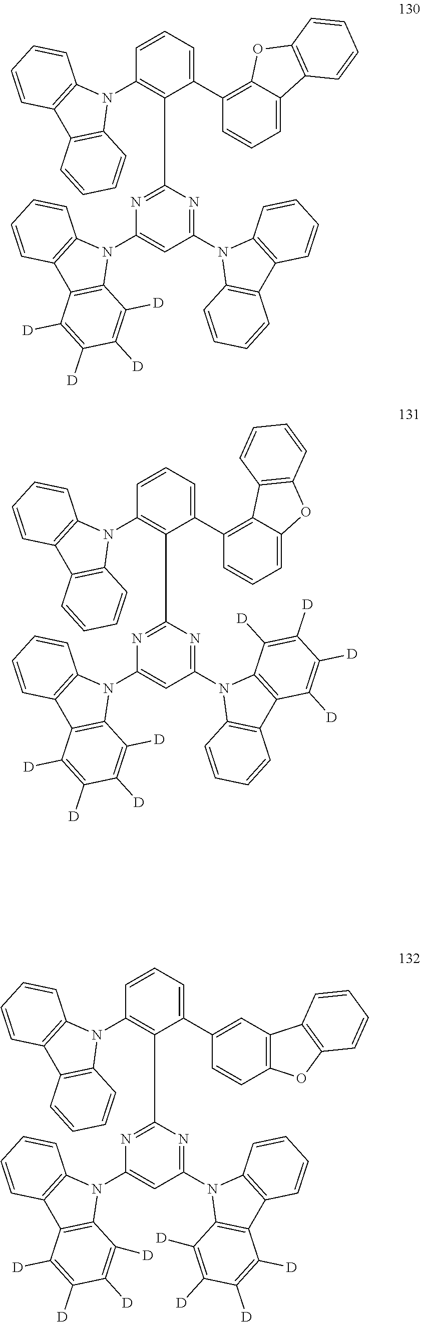 [SMILES: [2H]c1c([2H])c([2H])c2c(c1[2H])c1ccccc1n2-c1cc(-n2c3ccccc3c3ccccc32)nc(-c2c(-c3cccc4c3oc3ccccc34)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-c3cccc4oc5ccccc5c34)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-c3ccc4oc5ccccc5c4c3)cccc2-n2c3ccccc3c3ccccc32)n1]

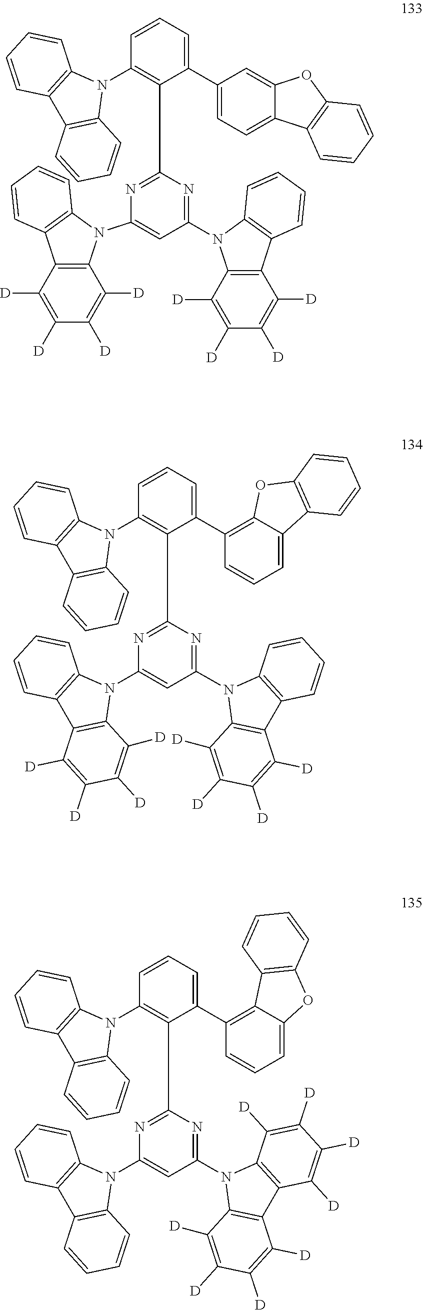 [SMILES: [2H]c1c([2H])c([2H])c2c(c1[2H])c1ccccc1n2-c1cc(-n2c3ccccc3c3c([2H])c([2H])c([2H])c([2H])c32)nc(-c2c(-c3ccc4c(c3)oc3ccccc34)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-c3cccc4c3oc3ccccc34)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3ccccc3c3ccccc32)nc(-c2c(-c3cccc4oc5ccccc5c34)cccc2-n2c3ccccc3c3ccccc32)n1]

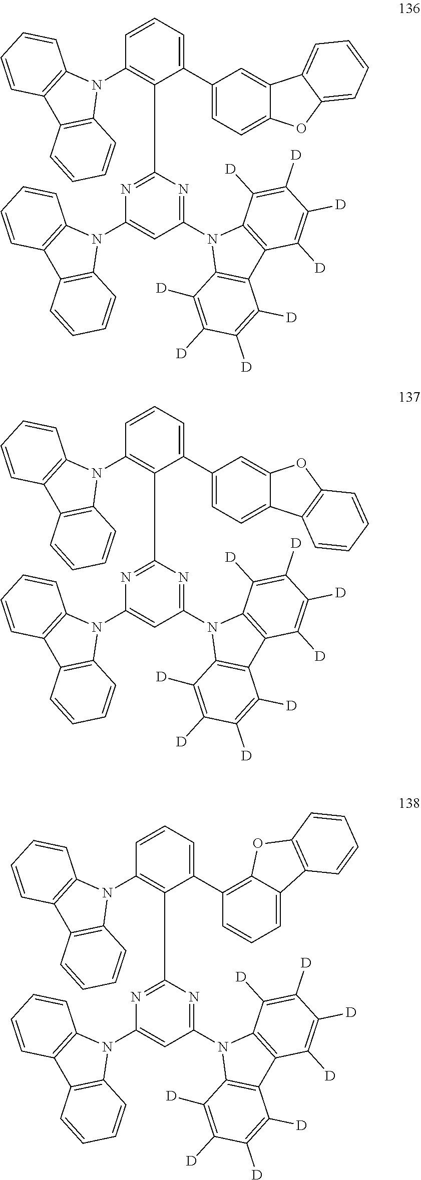 [SMILES: [2H]c1c([2H])c([2H])c2c(c1[2H])c1c([2H])c([2H])c([2H])c([2H])c1n2-c1cc(-n2c3ccccc3c3ccccc32)nc(-c2c(-c3ccc4oc5ccccc5c4c3)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3ccccc3c3ccccc32)nc(-c2c(-c3ccc4c(c3)oc3ccccc34)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3ccccc3c3ccccc32)nc(-c2c(-c3cccc4c3oc3ccccc34)cccc2-n2c3ccccc3c3ccccc32)n1]

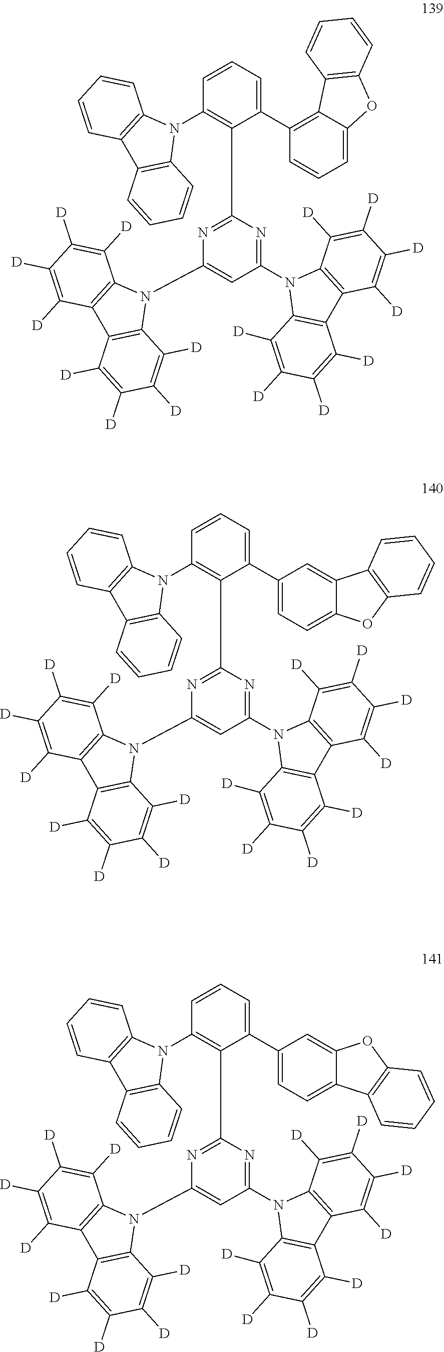

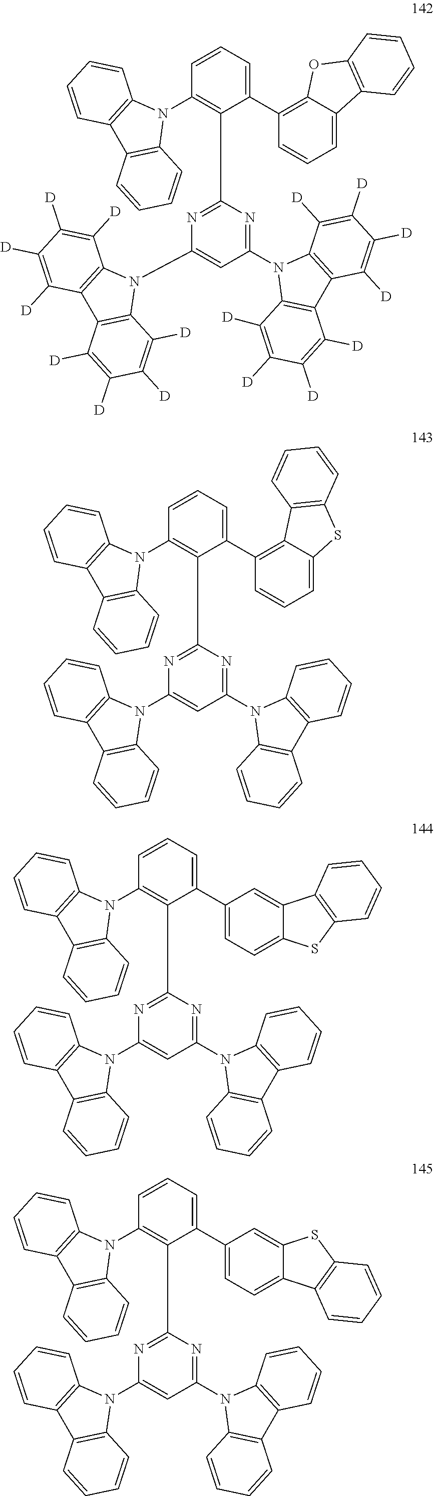

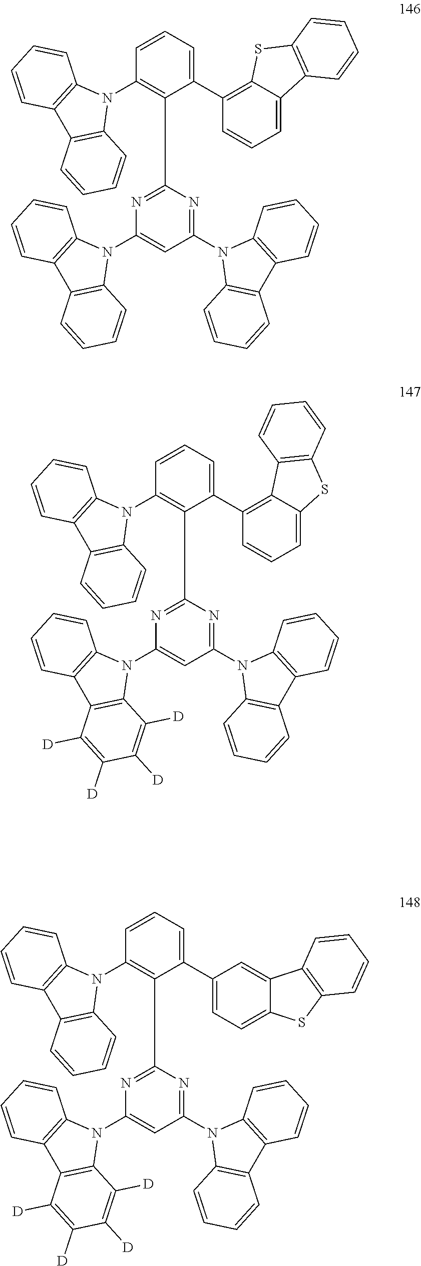 [SMILES: c1cc(-c2cccc3c2sc2ccccc23)c(-c2nc(-n3c4ccccc4c4ccccc43)cc(-n3c4ccccc4c4ccccc43)n2)c(-n2c3ccccc3c3ccccc32)c1 ; [2H]c1c([2H])c([2H])c2c(c1[2H])c1ccccc1n2-c1cc(-n2c3ccccc3c3ccccc32)nc(-c2c(-c3cccc4sc5ccccc5c34)cccc2-n2c3ccccc3c3ccccc32)n1 ; [2H]c1c([2H])c([2H])c2c(c1[2H])c1ccccc1n2-c1cc(-n2c3ccccc3c3ccccc32)nc(-c2c(-c3ccc4sc5ccccc5c4c3)cccc2-n2c3ccccc3c3ccccc32)n1]

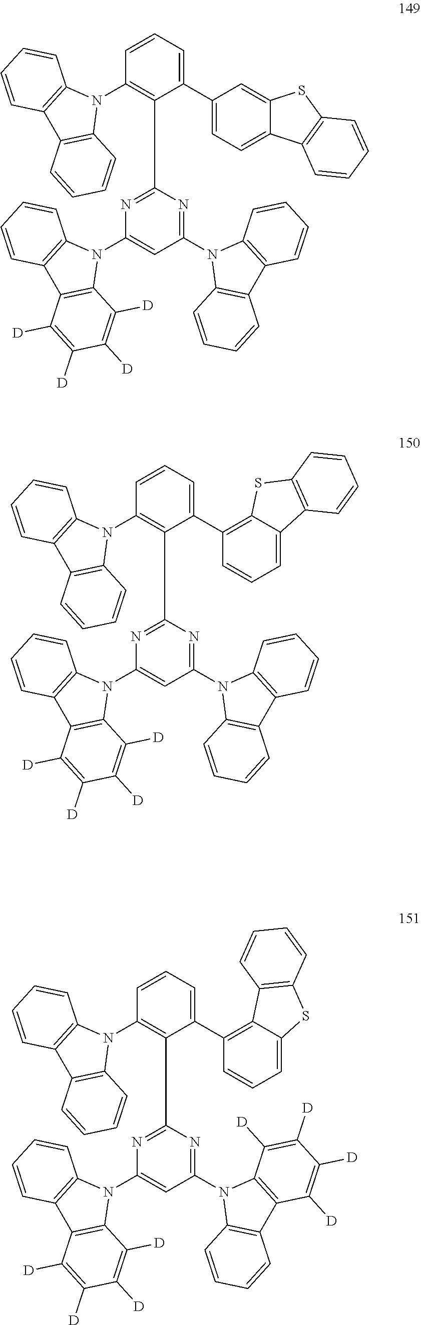 [SMILES: [2H]c1c([2H])c([2H])c2c(c1[2H])c1ccccc1n2-c1cc(-n2c3ccccc3c3ccccc32)nc(-c2c(-c3ccc4c(c3)sc3ccccc34)cccc2-n2c3ccccc3c3ccccc32)n1 ; [2H]c1c([2H])c([2H])c2c(c1[2H])c1ccccc1n2-c1cc(-n2c3ccccc3c3ccccc32)nc(-c2c(-c3cccc4c3sc3ccccc34)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-c3cccc4sc5ccccc5c34)cccc2-n2c3ccccc3c3ccccc32)n1]

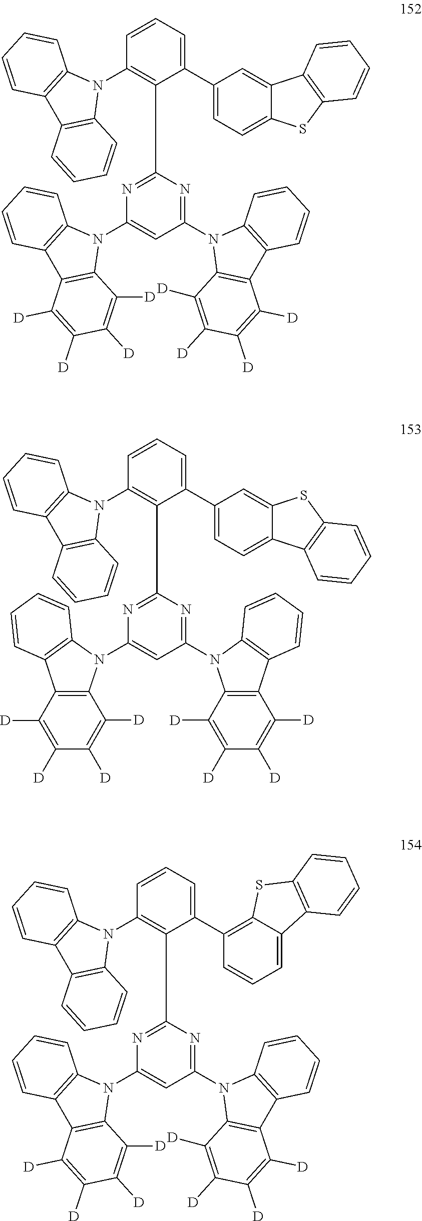 [SMILES: [2H]c1c([2H])c([2H])c2c(c1[2H])c1ccccc1n2-c1cc(-n2c3ccccc3c3c([2H])c([2H])c([2H])c([2H])c32)nc(-c2c(-c3ccc4sc5ccccc5c4c3)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-c3ccc4c(c3)sc3ccccc34)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-c3cccc4c3sc3ccccc34)cccc2-n2c3ccccc3c3ccccc32)n1]

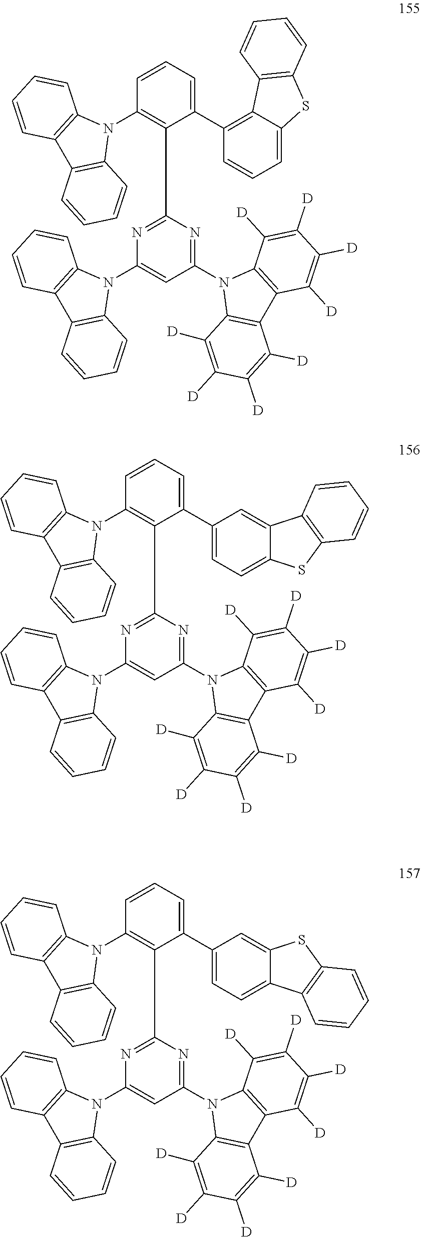 [SMILES: [2H]c1c([2H])c([2H])c2c(c1[2H])c1c([2H])c([2H])c([2H])c([2H])c1n2-c1cc(-n2c3ccccc3c3ccccc32)nc(-c2c(-c3cccc4sc5ccccc5c34)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3ccccc3c3ccccc32)nc(-c2c(-c3ccc4sc5ccccc5c4c3)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3ccccc3c3ccccc32)nc(-c2c(-c3ccc4c(c3)sc3ccccc34)cccc2-n2c3ccccc3c3ccccc32)n1]

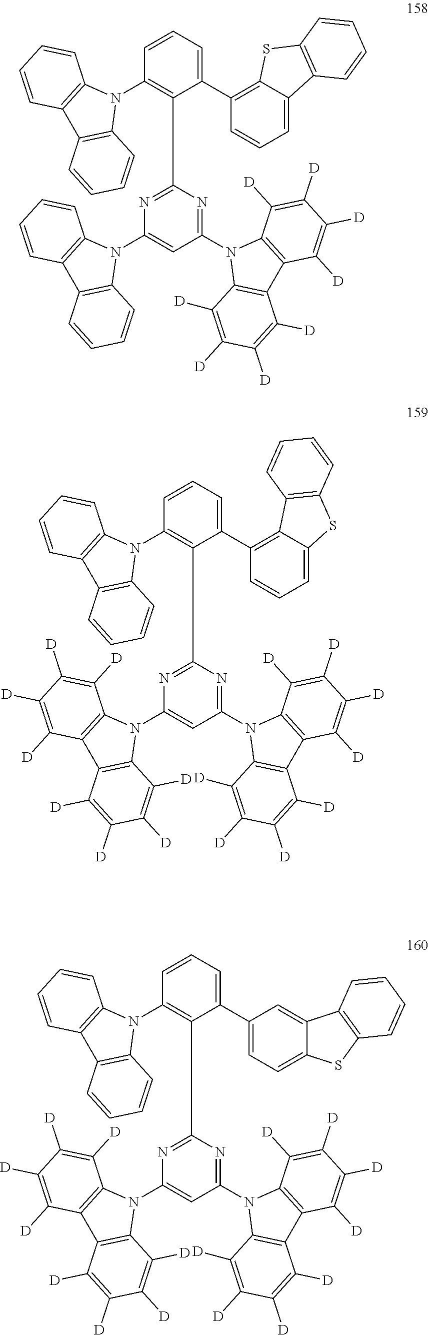

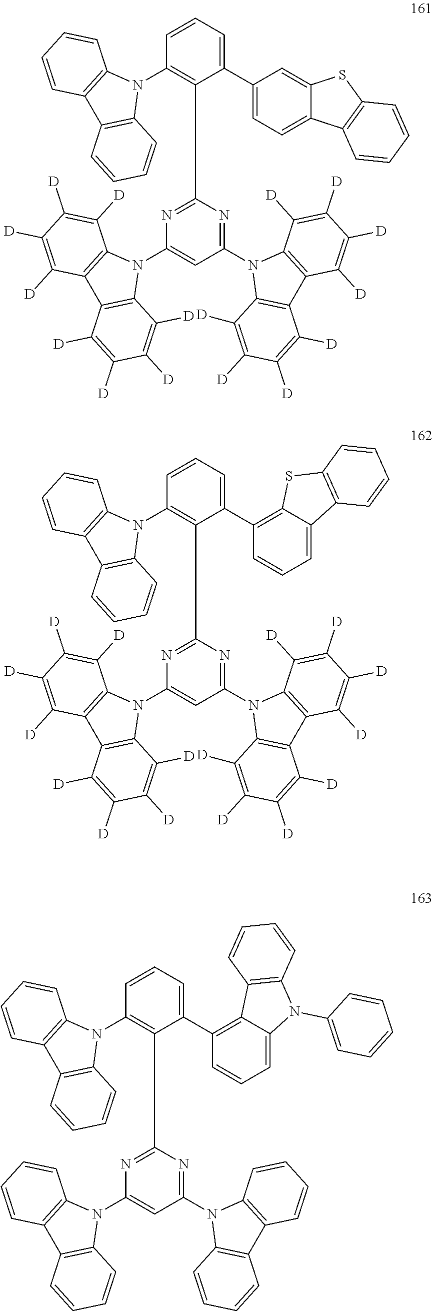

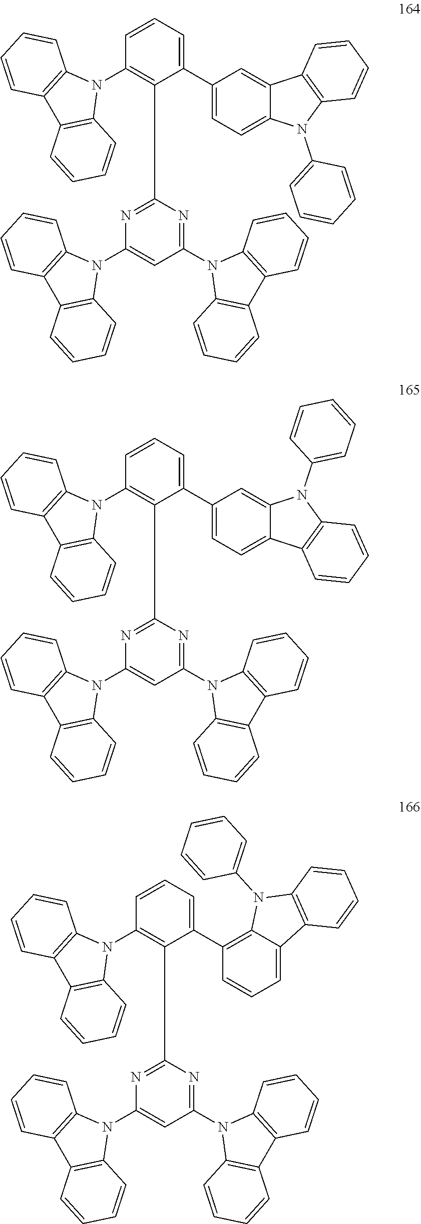

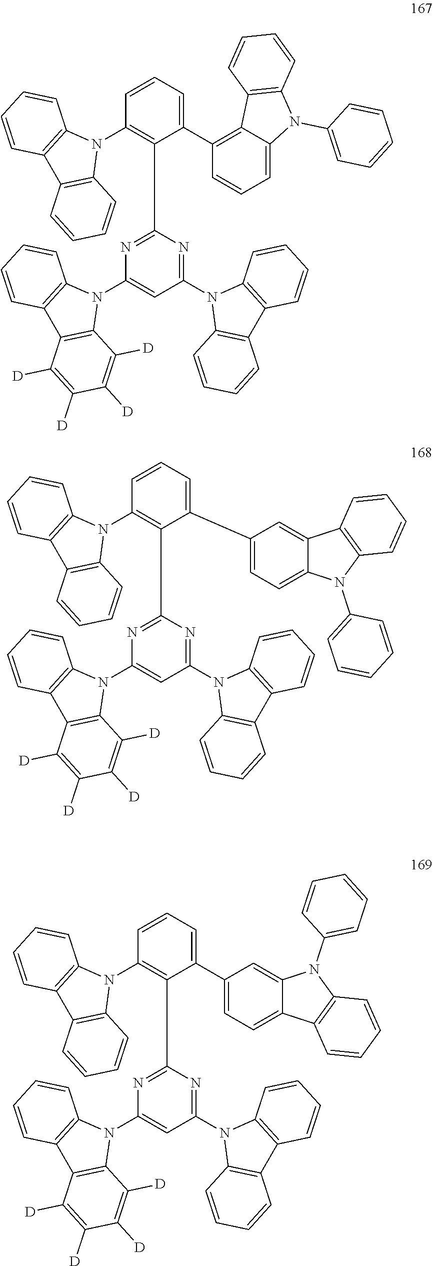 [SMILES: [2H]c1c([2H])c([2H])c2c(c1[2H])c1ccccc1n2-c1cc(-n2c3ccccc3c3ccccc32)nc(-c2c(-c3cccc4c3c3ccccc3n4-c3ccccc3)cccc2-n2c3ccccc3c3ccccc32)n1 ; [2H]c1c([2H])c([2H])c2c(c1[2H])c1ccccc1n2-c1cc(-n2c3ccccc3c3ccccc32)nc(-c2c(-c3ccc4c(c3)c3ccccc3n4-c3ccccc3)cccc2-n2c3ccccc3c3ccccc32)n1 ; [2H]c1c([2H])c([2H])c2c(c1[2H])c1ccccc1n2-c1cc(-n2c3ccccc3c3ccccc32)nc(-c2c(-c3ccc4c5ccccc5n(-c5ccccc5)c4c3)cccc2-n2c3ccccc3c3ccccc32)n1]

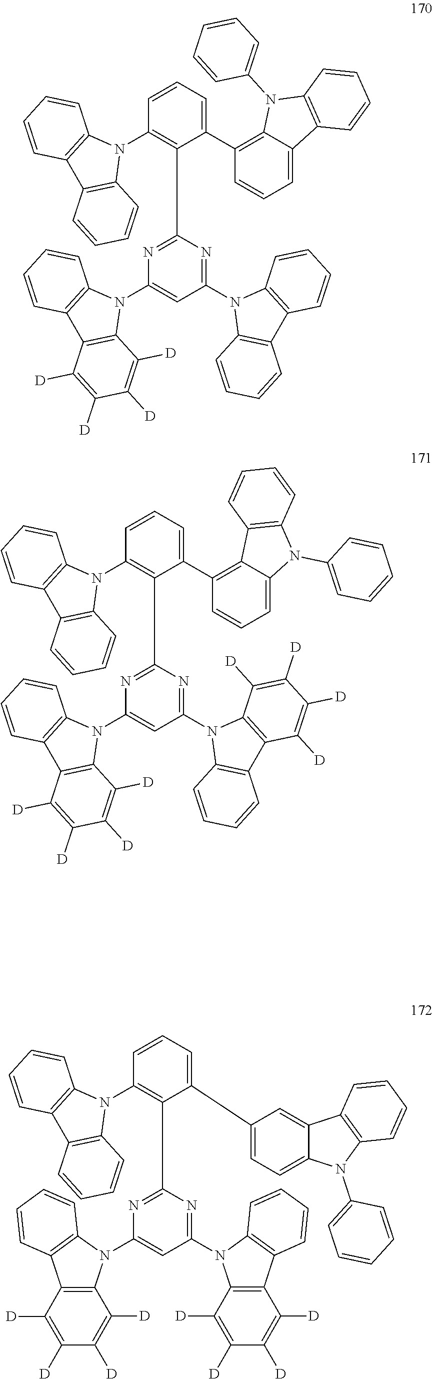 [SMILES: [2H]c1c([2H])c([2H])c2c(c1[2H])c1ccccc1n2-c1cc(-n2c3ccccc3c3ccccc32)nc(-c2c(-c3cccc4c5ccccc5n(-c5ccccc5)c34)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-c3cccc4c3c3ccccc3n4-c3ccccc3)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-c3ccc4c(c3)c3ccccc3n4-c3ccccc3)cccc2-n2c3ccccc3c3ccccc32)n1]

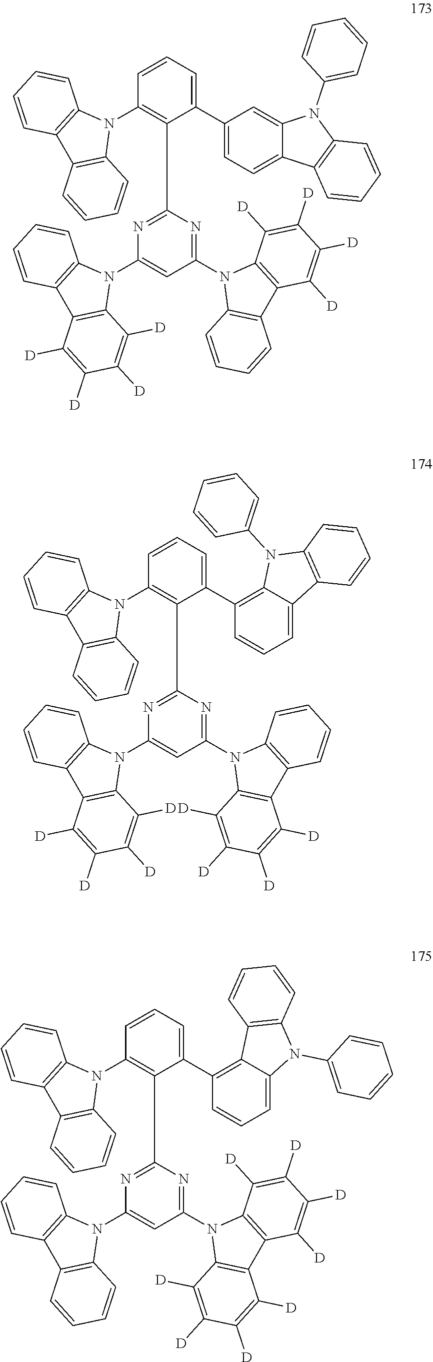 [SMILES: [2H]c1c([2H])c([2H])c2c(c1[2H])c1ccccc1n2-c1cc(-n2c3ccccc3c3c([2H])c([2H])c([2H])c([2H])c32)nc(-c2c(-c3ccc4c5ccccc5n(-c5ccccc5)c4c3)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-c3cccc4c5ccccc5n(-c5ccccc5)c34)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3ccccc3c3ccccc32)nc(-c2c(-c3cccc4c3c3ccccc3n4-c3ccccc3)cccc2-n2c3ccccc3c3ccccc32)n1]

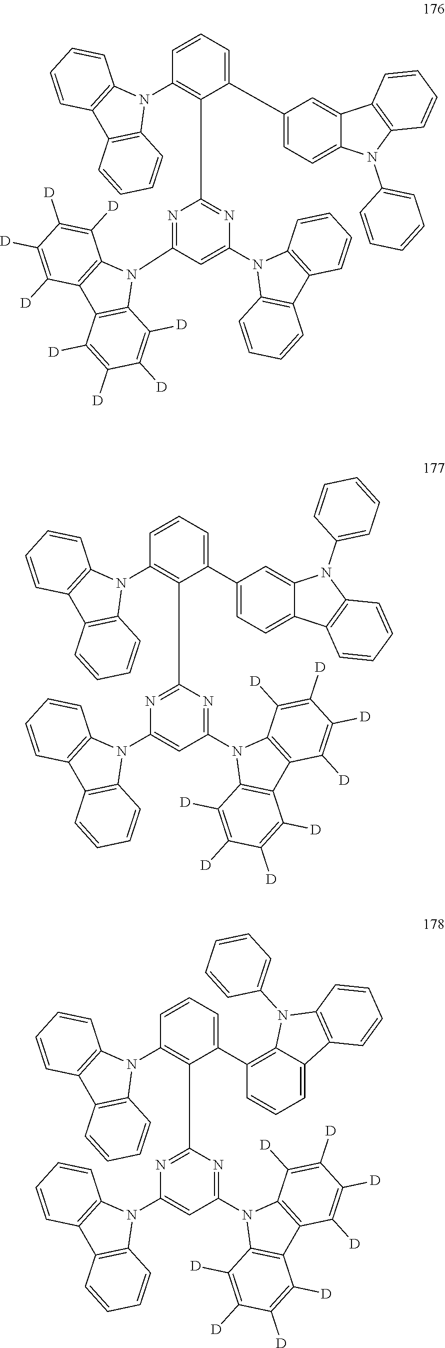 [SMILES: [2H]c1c([2H])c([2H])c2c(c1[2H])c1c([2H])c([2H])c([2H])c([2H])c1n2-c1cc(-n2c3ccccc3c3ccccc32)nc(-c2c(-c3ccc4c(c3)c3ccccc3n4-c3ccccc3)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3ccccc3c3ccccc32)nc(-c2c(-c3ccc4c5ccccc5n(-c5ccccc5)c4c3)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3ccccc3c3ccccc32)nc(-c2c(-c3cccc4c5ccccc5n(-c5ccccc5)c34)cccc2-n2c3ccccc3c3ccccc32)n1]

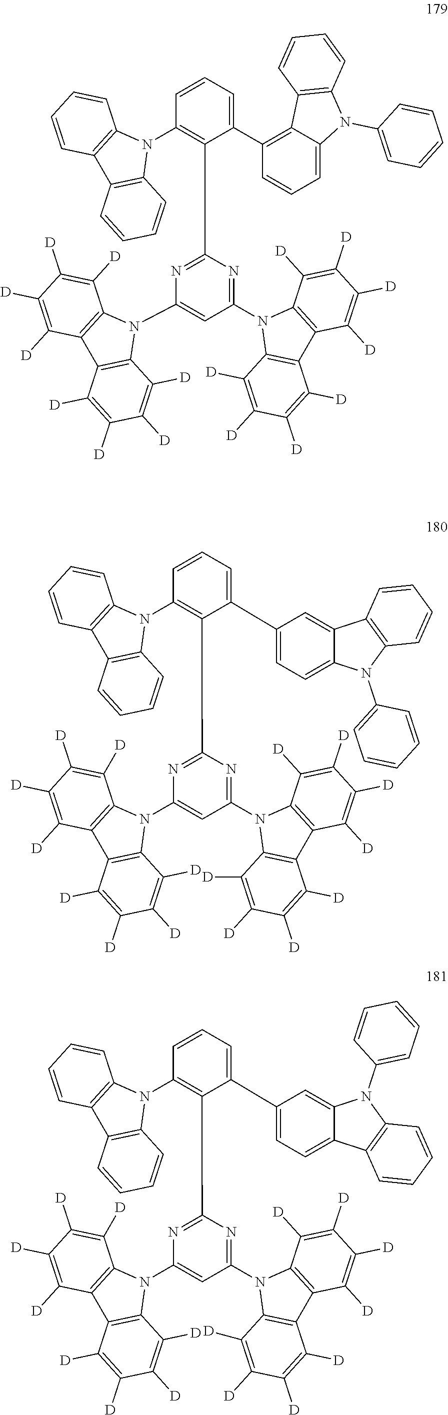

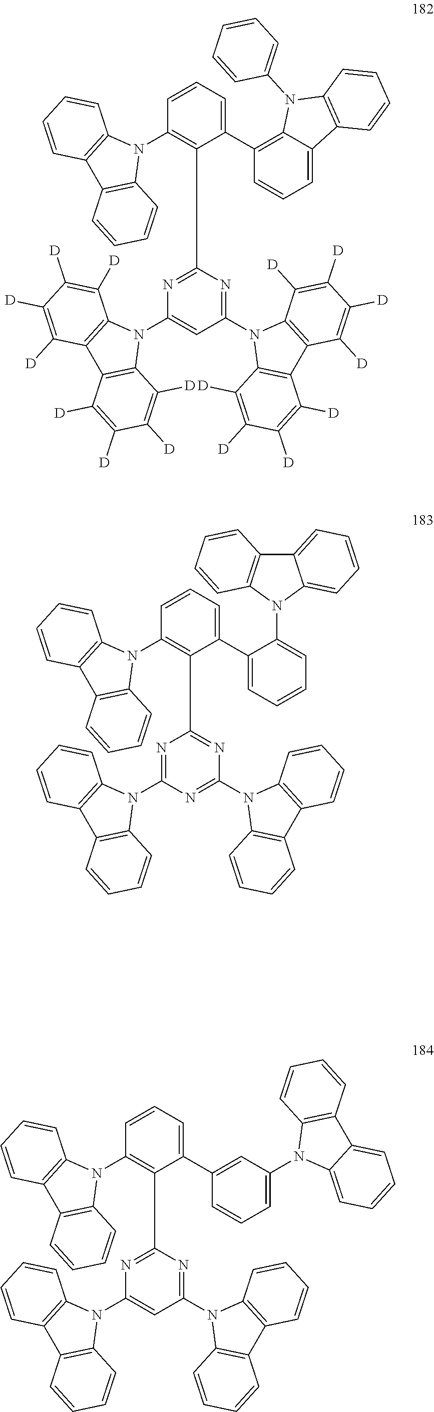 [SMILES: [2H]c1c([2H])c([2H])c2c(c1[2H])c1c([2H])c([2H])c([2H])c([2H])c1n2-c1cc(-n2c3c([2H])c([2H])c([2H])c([2H])c3c3c([2H])c([2H])c([2H])c([2H])c32)nc(-c2c(-c3cccc4c5ccccc5n(-c5ccccc5)c34)cccc2-n2c3ccccc3c3ccccc32)n1 ; c1ccc(-n2c3ccccc3c3ccccc32)c(-c2cccc(-n3c4ccccc4c4ccccc43)c2-c2nc(-n3c4ccccc4c4ccccc43)nc(-n3c4ccccc4c4ccccc43)n2)c1 ; c1cc(-c2cccc(-n3c4ccccc4c4ccccc43)c2-c2nc(-n3c4ccccc4c4ccccc43)cc(-n3c4ccccc4c4ccccc43)n2)cc(-n2c3ccccc3c3ccccc32)c1]

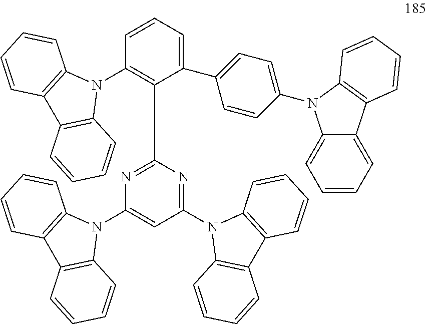 [SMILES: c1cc(-c2ccc(-n3c4ccccc4c4ccccc43)cc2)c(-c2nc(-n3c4ccccc4c4ccccc43)cc(-n3c4ccccc4c4ccccc43)n2)c(-n2c3ccccc3c3ccccc32)c1]

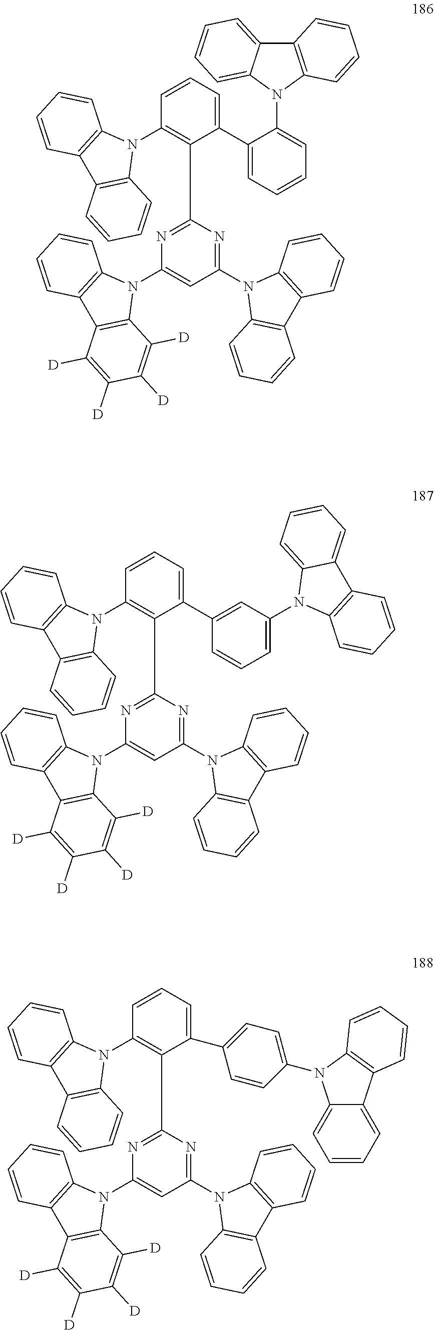 [SMILES: [2H]c1c([2H])c([2H])c2c(c1[2H])c1ccccc1n2-c1cc(-n2c3ccccc3c3ccccc32)nc(-c2c(-c3ccccc3-n3c4ccccc4c4ccccc43)cccc2-n2c3ccccc3c3ccccc32)n1 ; [2H]c1c([2H])c([2H])c2c(c1[2H])c1ccccc1n2-c1cc(-n2c3ccccc3c3ccccc32)nc(-c2c(-c3cccc(-n4c5ccccc5c5ccccc54)c3)cccc2-n2c3ccccc3c3ccccc32)n1 ; [2H]c1c([2H])c([2H])c2c(c1[2H])c1ccccc1n2-c1cc(-n2c3ccccc3c3ccccc32)nc(-c2c(-c3ccc(-n4c5ccccc5c5ccccc54)cc3)cccc2-n2c3ccccc3c3ccccc32)n1]

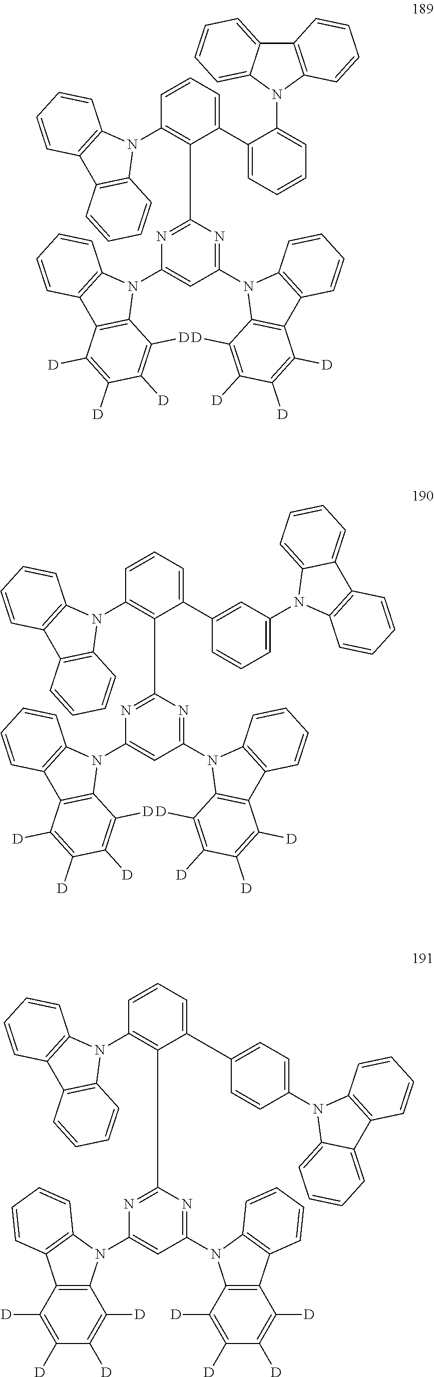 [SMILES: [2H]c1c([2H])c([2H])c2c(c1[2H])c1ccccc1n2-c1cc(-n2c3ccccc3c3c([2H])c([2H])c([2H])c([2H])c32)nc(-c2c(-c3ccccc3-n3c4ccccc4c4ccccc43)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-c3cccc(-n4c5ccccc5c5ccccc54)c3)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-c3ccc(-n4c5ccccc5c5ccccc54)cc3)cccc2-n2c3ccccc3c3ccccc32)n1]

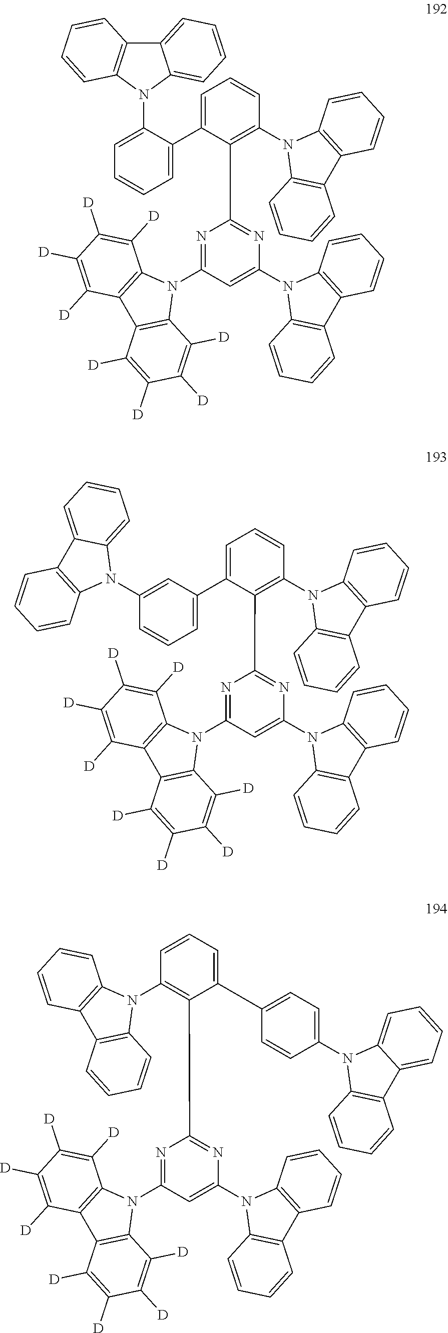 [SMILES: [2H]c1c([2H])c([2H])c2c(c1[2H])c1c([2H])c([2H])c([2H])c([2H])c1n2-c1cc(-n2c3ccccc3c3ccccc32)nc(-c2c(-c3ccccc3-n3c4ccccc4c4ccccc43)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3ccccc3c3ccccc32)nc(-c2c(-c3cccc(-n4c5ccccc5c5ccccc54)c3)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3ccccc3c3ccccc32)nc(-c2c(-c3ccc(-n4c5ccccc5c5ccccc54)cc3)cccc2-n2c3ccccc3c3ccccc32)n1]

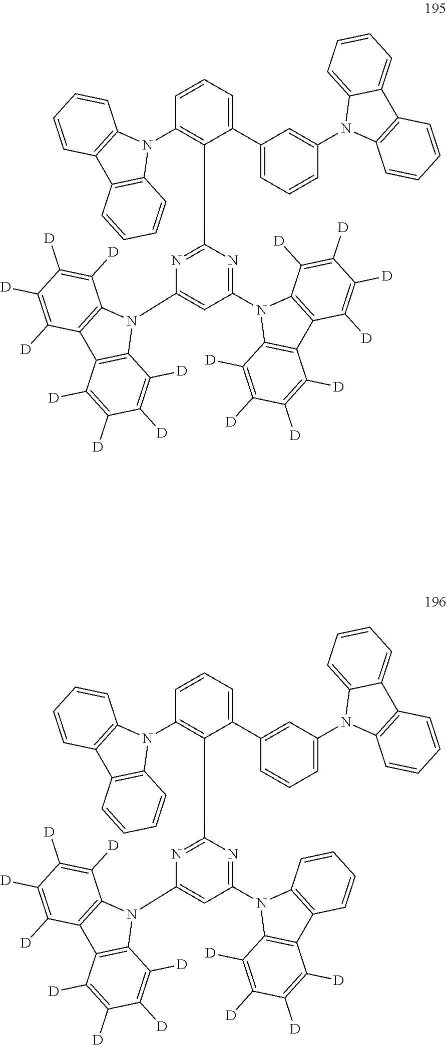 [SMILES: [2H]c1c([2H])c([2H])c2c(c1[2H])c1c([2H])c([2H])c([2H])c([2H])c1n2-c1cc(-n2c3c([2H])c([2H])c([2H])c([2H])c3c3c([2H])c([2H])c([2H])c([2H])c32)nc(-c2c(-c3cccc(-n4c5ccccc5c5ccccc54)c3)cccc2-n2c3ccccc3c3ccccc32)n1 ; [2H]c1c([2H])c([2H])c2c(c1[2H])c1ccccc1n2-c1cc(-n2c3c([2H])c([2H])c([2H])c([2H])c3c3c([2H])c([2H])c([2H])c([2H])c32)nc(-c2c(-c3cccc(-n4c5ccccc5c5ccccc54)c3)cccc2-n2c3ccccc3c3ccccc32)n1]

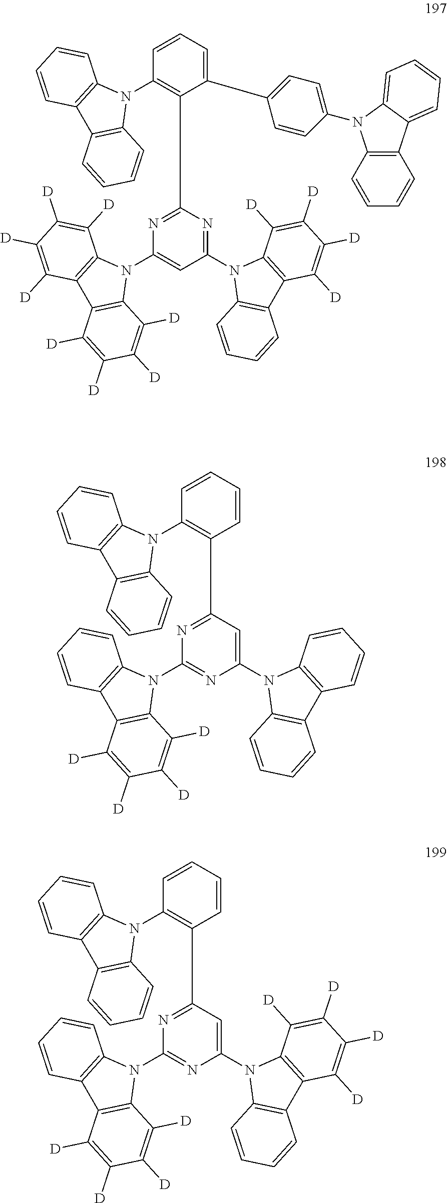 [SMILES: [2H]c1c([2H])c([2H])c2c(c1[2H])c1ccccc1n2-c1cc(-n2c3c([2H])c([2H])c([2H])c([2H])c3c3c([2H])c([2H])c([2H])c([2H])c32)nc(-c2c(-c3ccc(-n4c5ccccc5c5ccccc54)cc3)cccc2-n2c3ccccc3c3ccccc32)n1 ; [2H]c1c([2H])c([2H])c2c(c1[2H])c1ccccc1n2-c1nc(-c2ccccc2-n2c3ccccc3c3ccccc32)cc(-n2c3ccccc3c3ccccc32)n1 ; [2H]c1c([2H])c([2H])c2c(c1[2H])c1ccccc1n2-c1cc(-c2ccccc2-n2c3ccccc3c3ccccc32)nc(-n2c3ccccc3c3c([2H])c([2H])c([2H])c([2H])c32)n1]

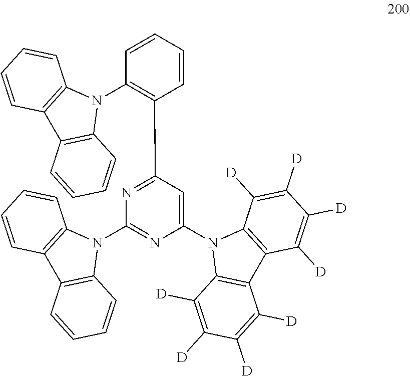 [SMILES: [2H]c1c([2H])c([2H])c2c(c1[2H])c1c([2H])c([2H])c([2H])c([2H])c1n2-c1cc(-c2ccccc2-n2c3ccccc3c3ccccc32)nc(-n2c3ccccc3c3ccccc32)n1]

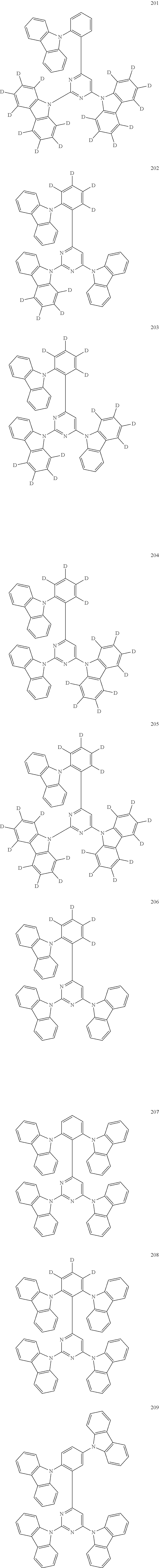

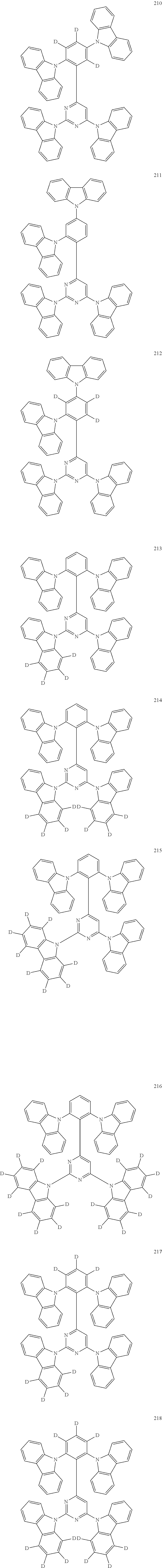

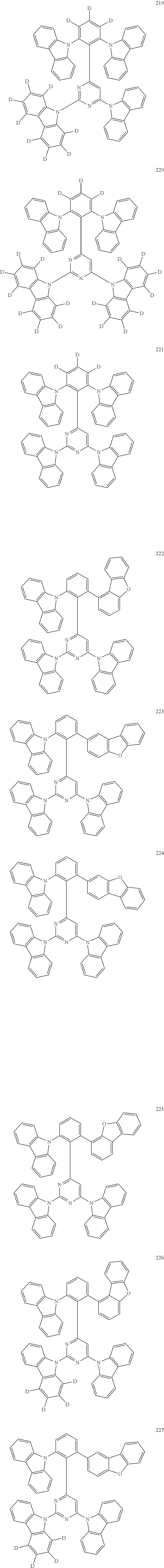

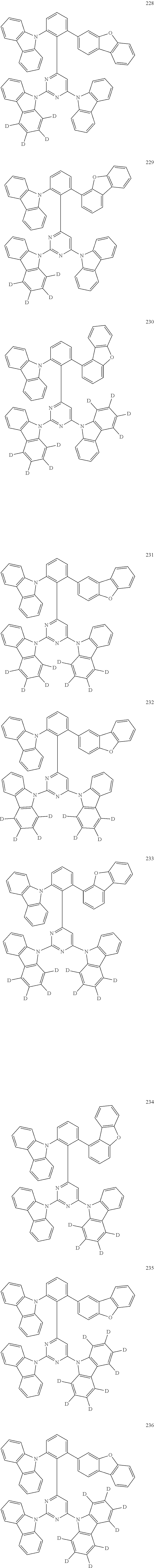

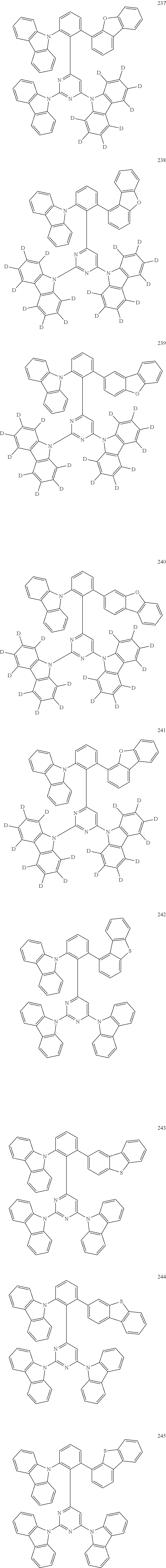

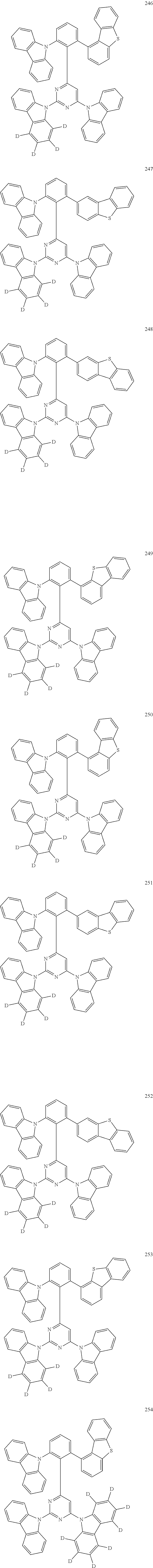

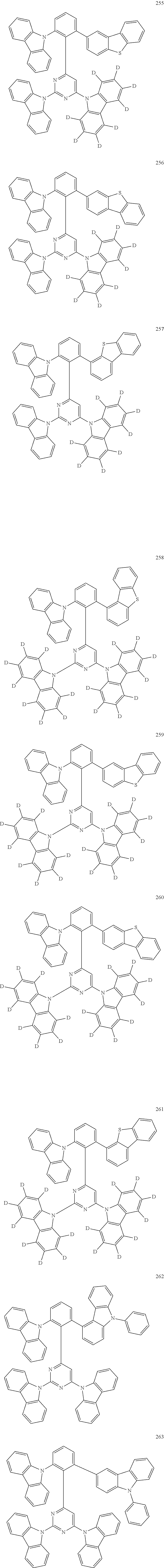

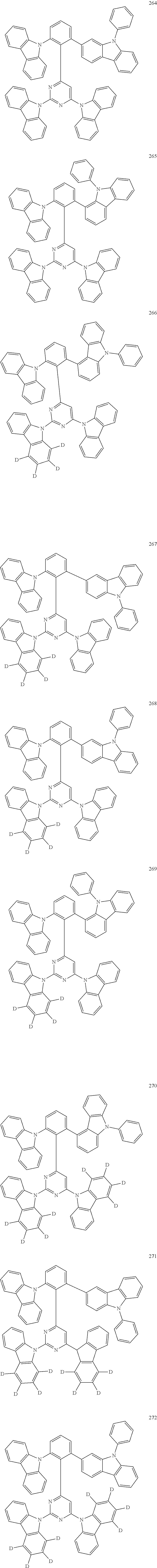

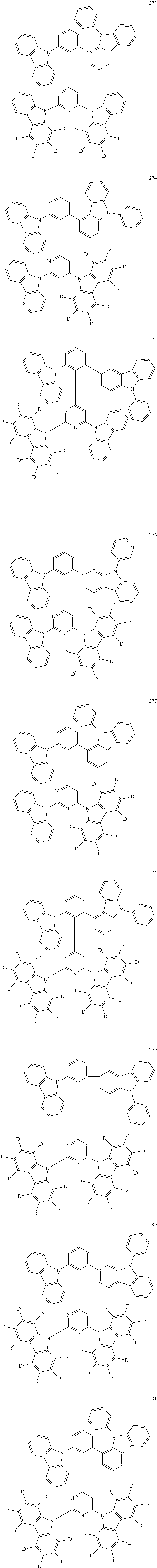

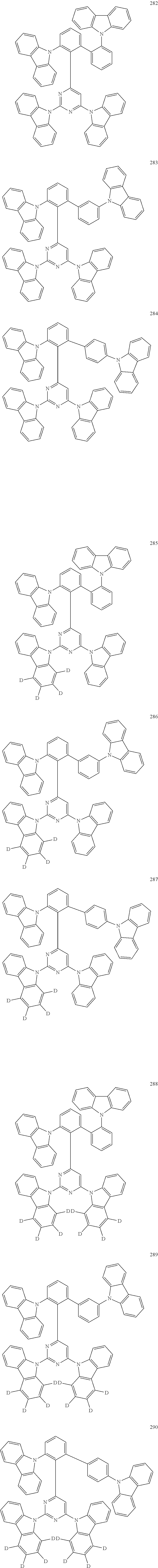

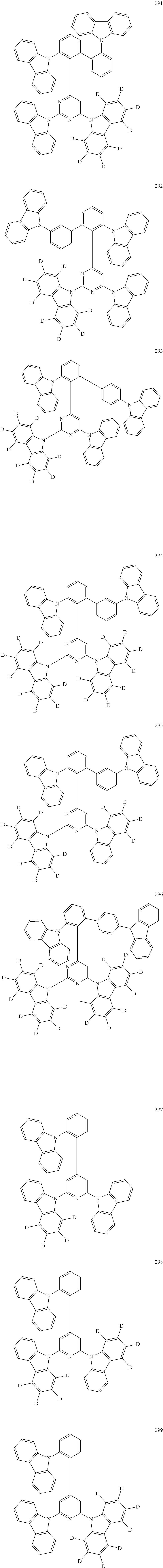

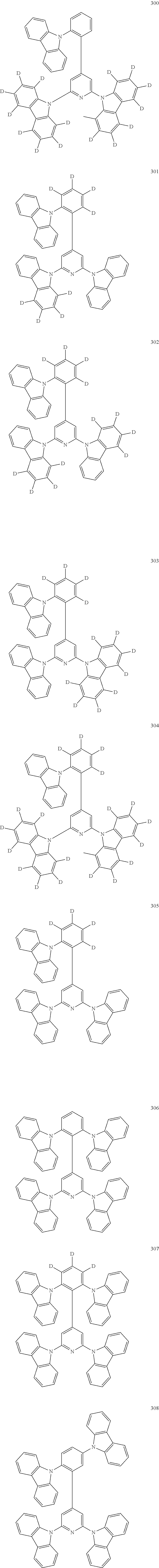

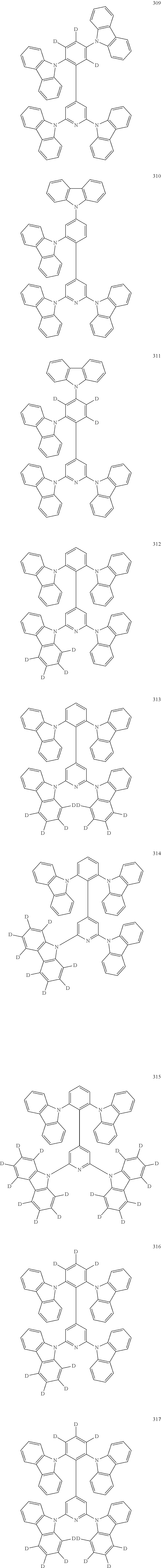

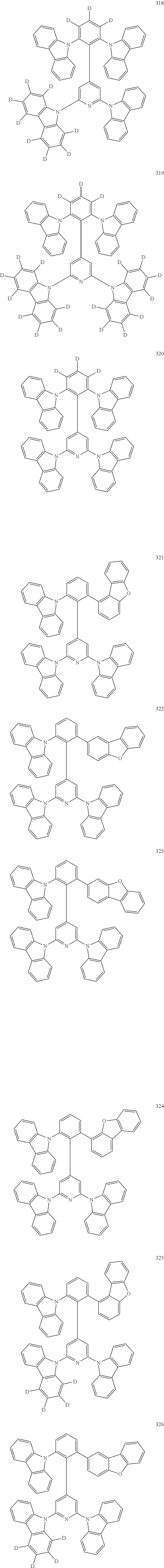

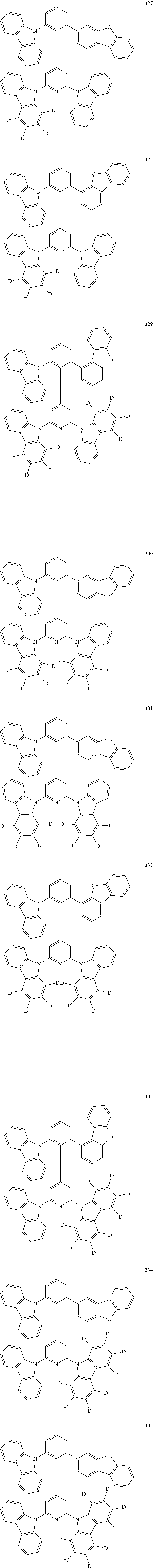

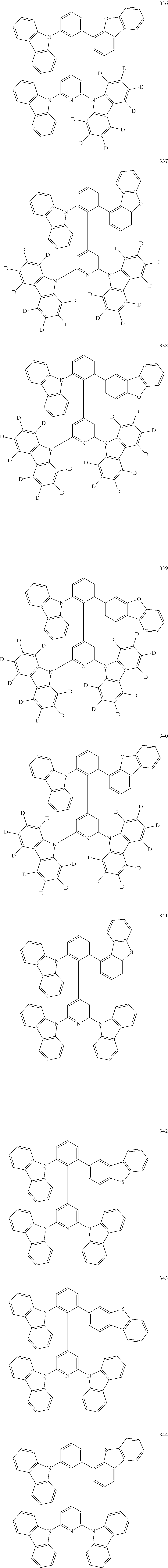

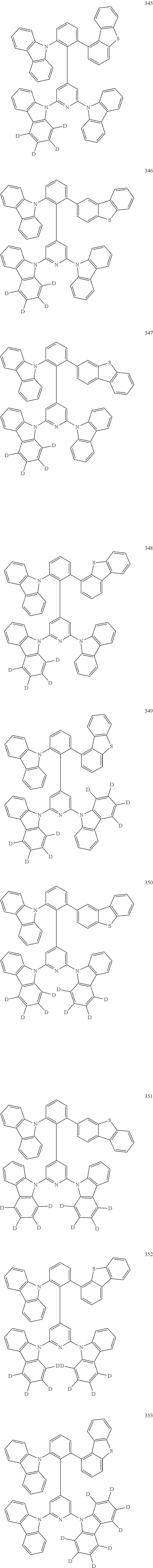

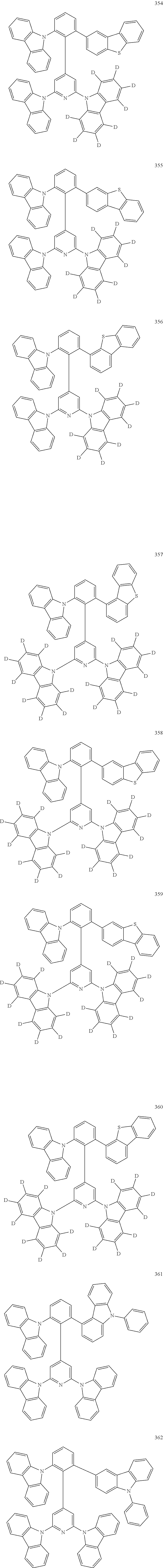

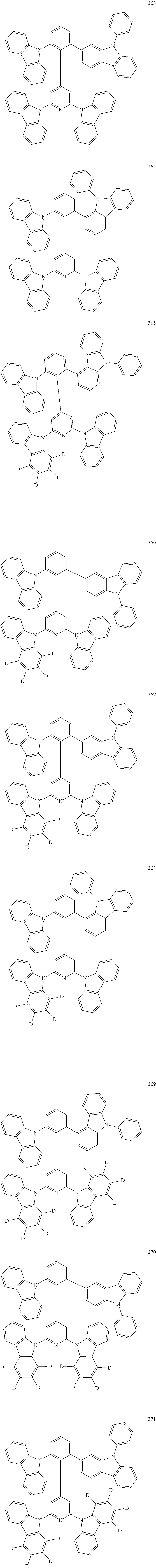

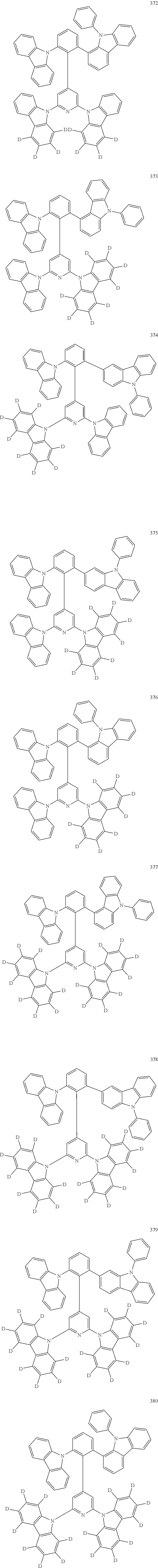

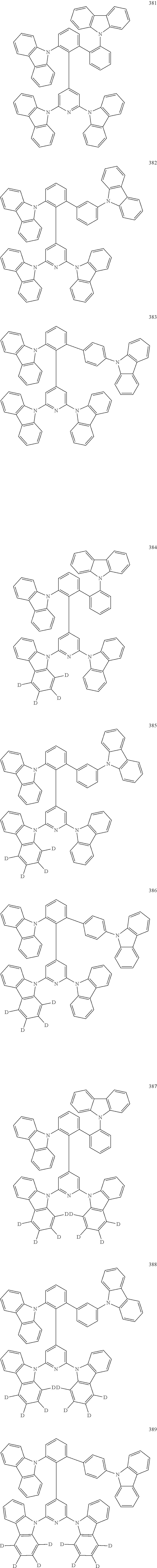

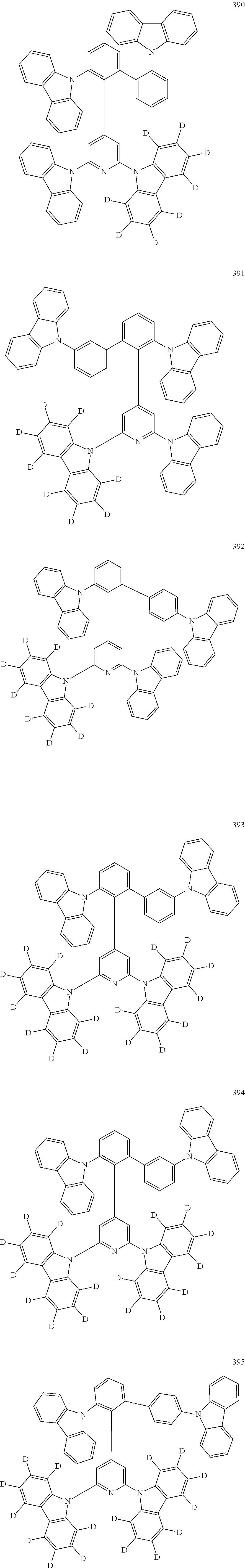

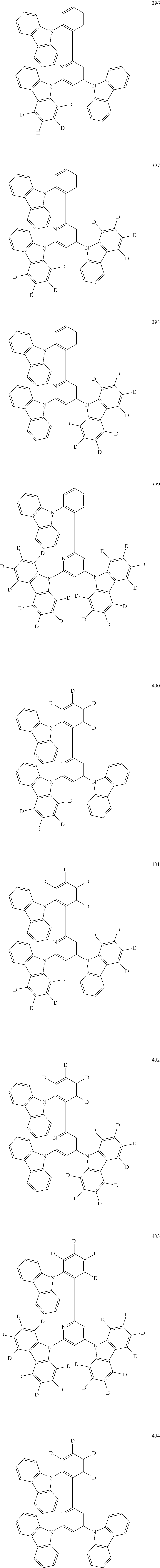

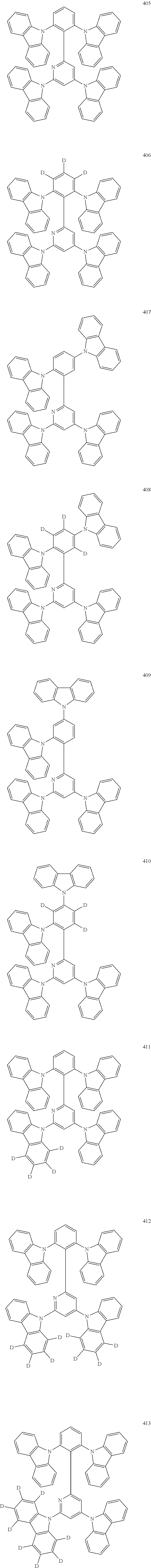

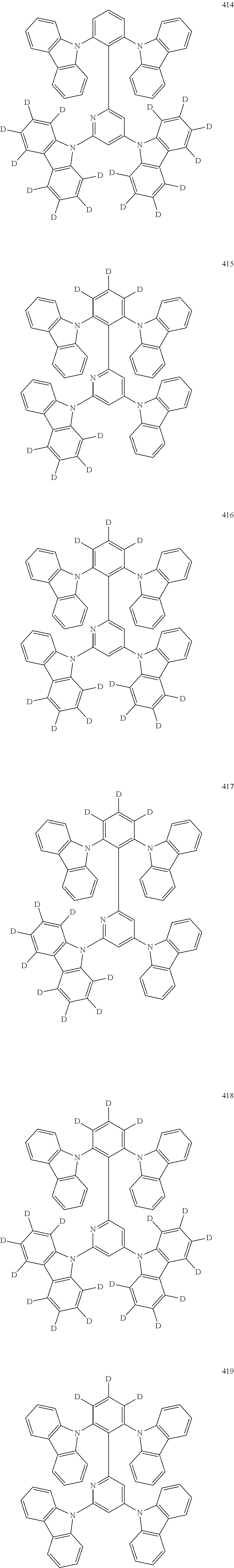

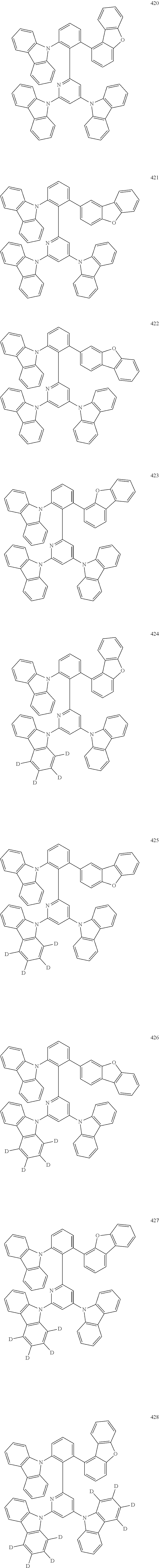

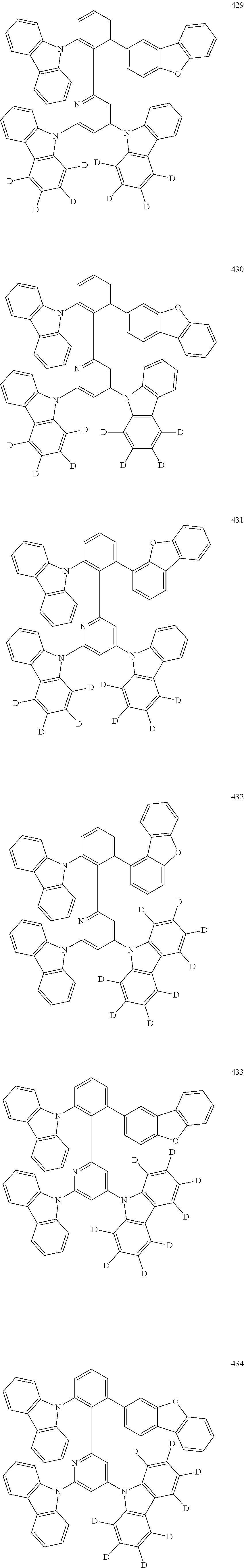

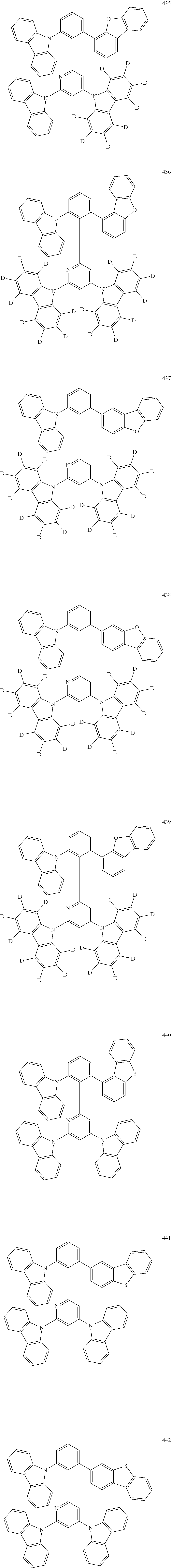

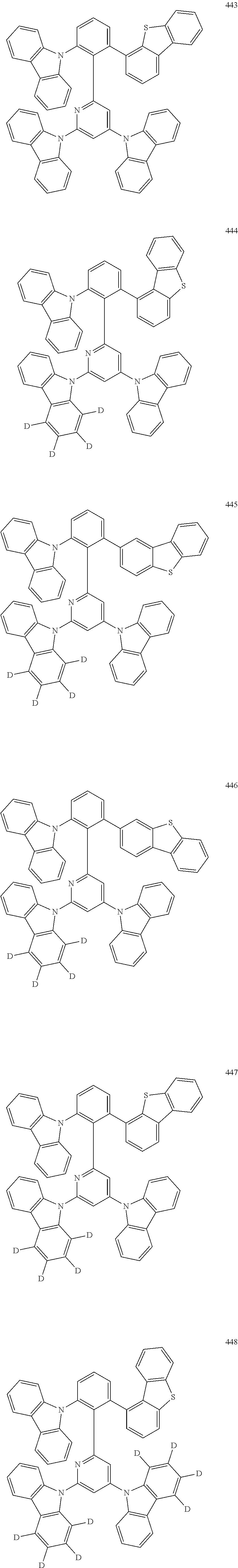

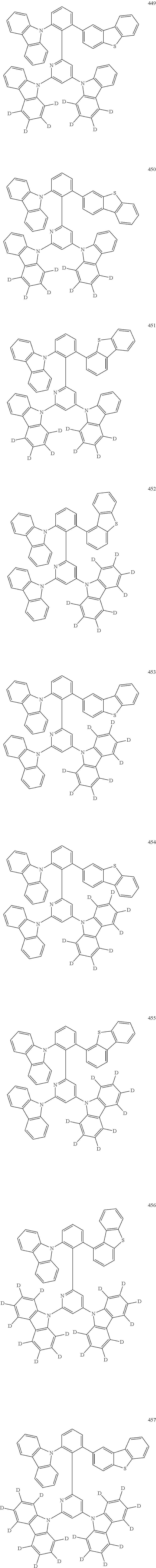

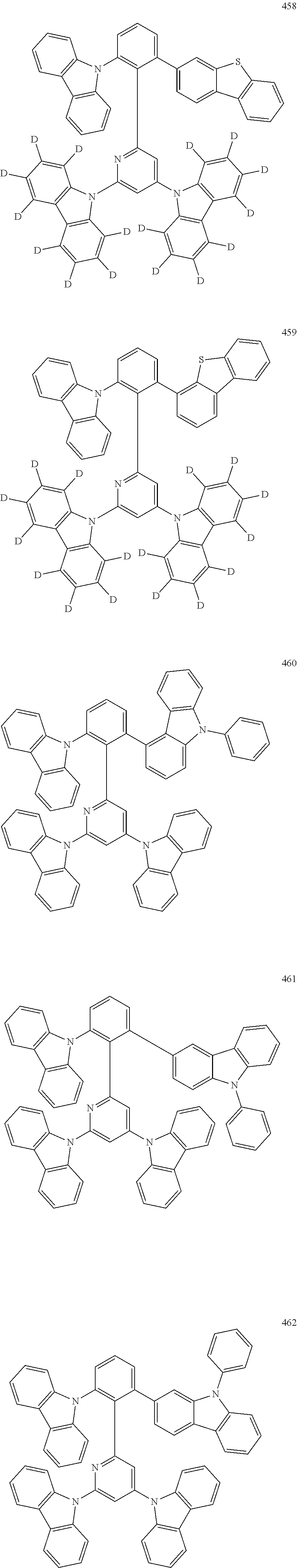

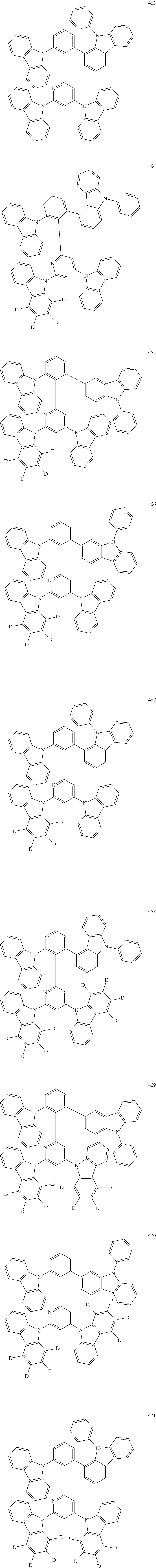

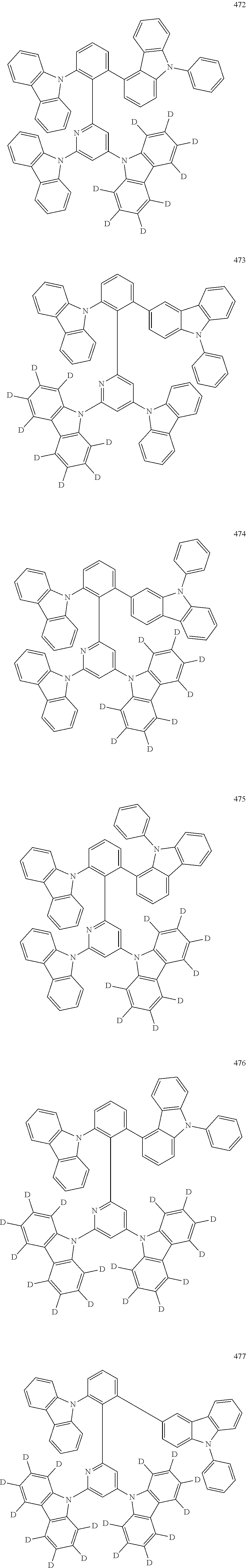

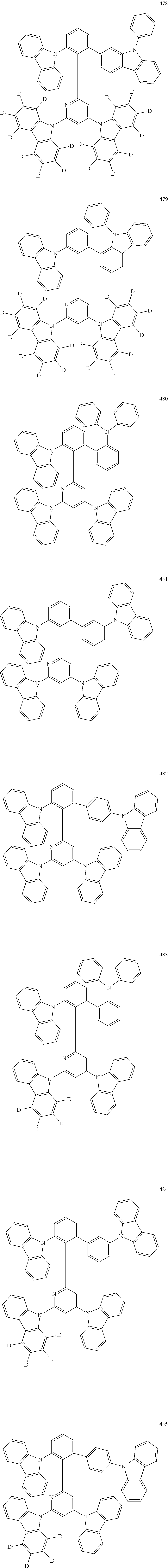

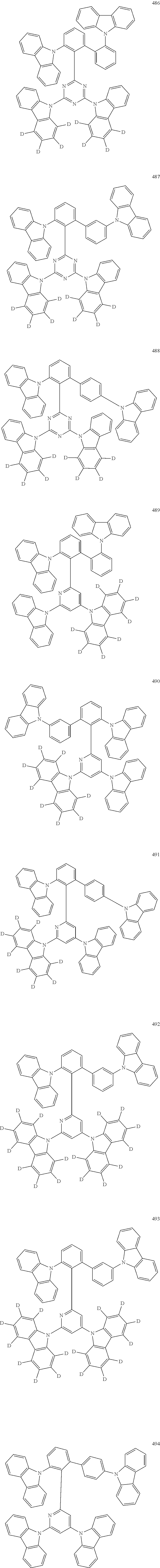

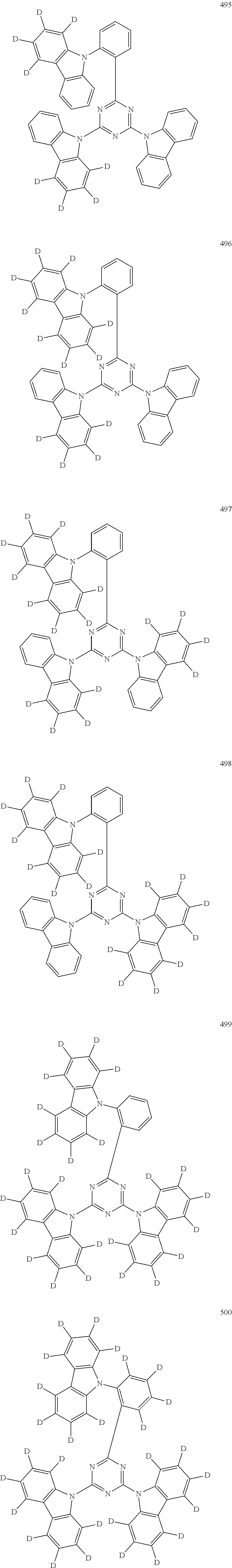

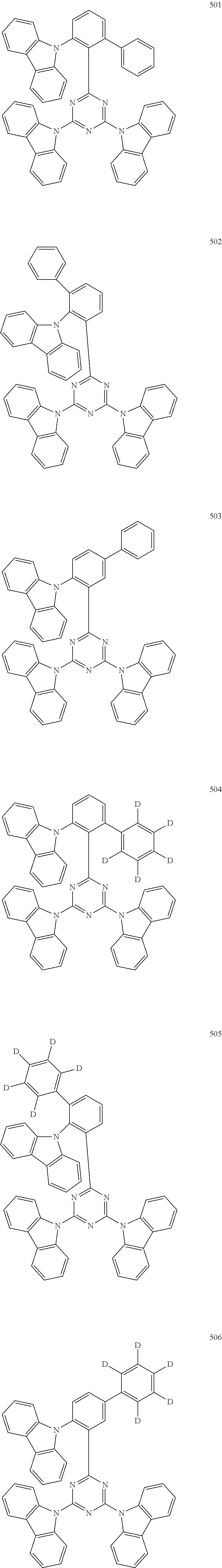

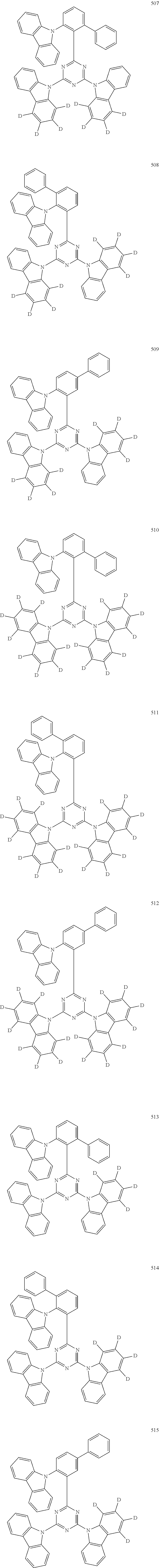

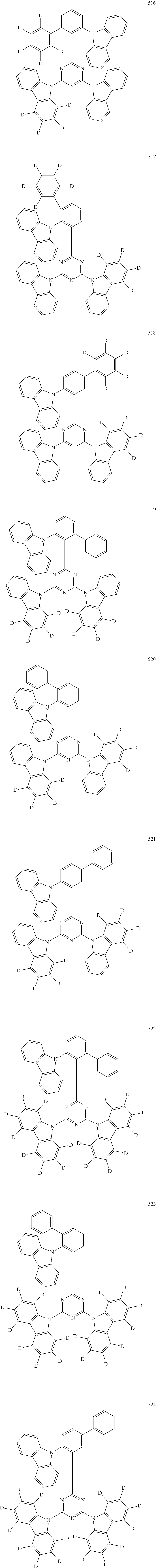

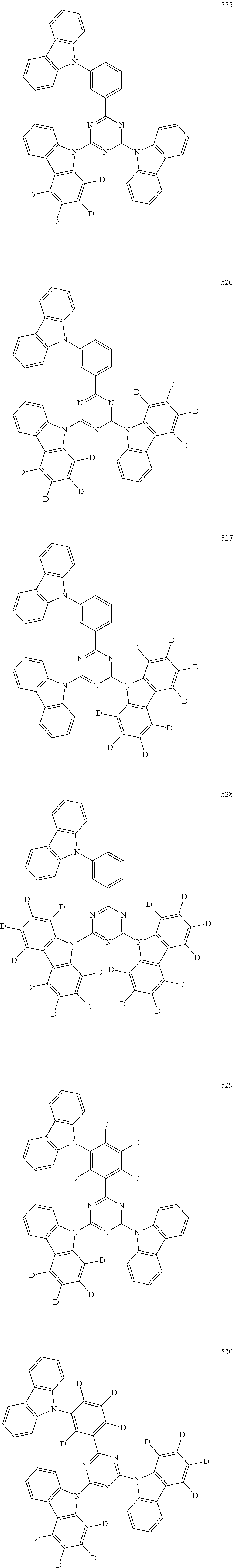

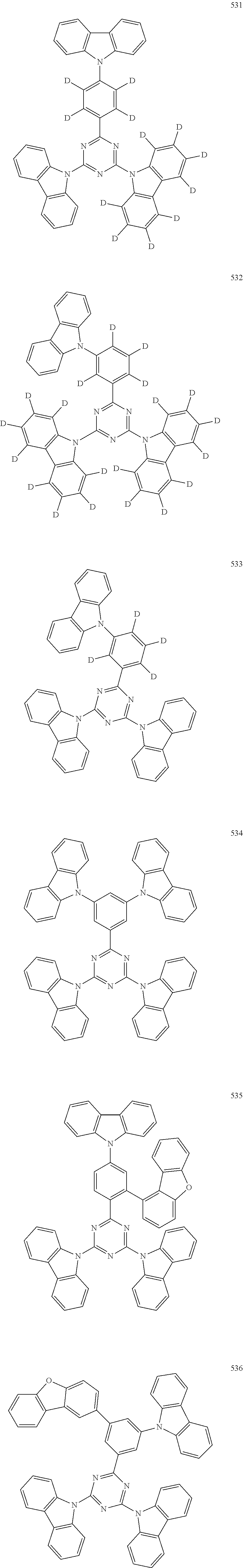

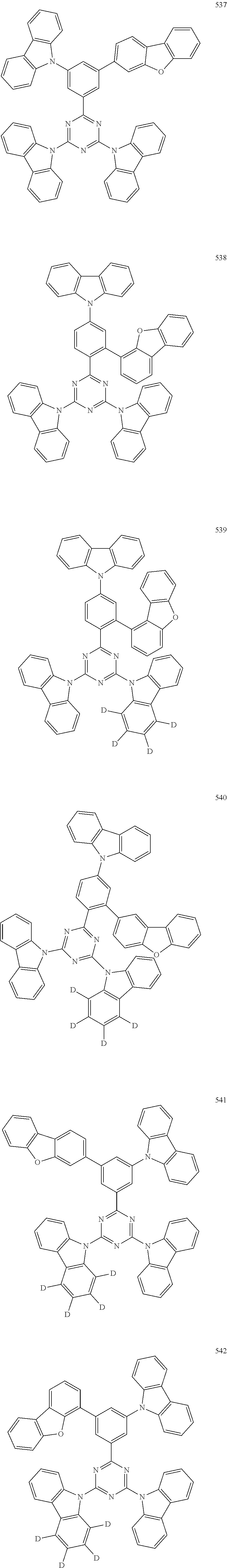

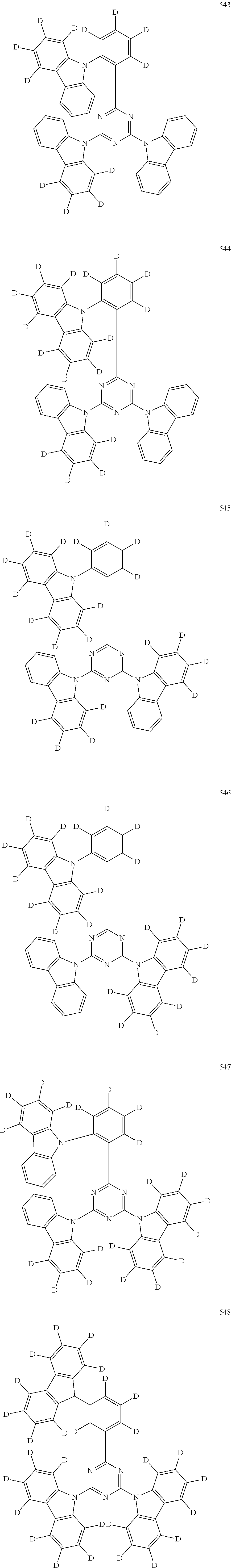

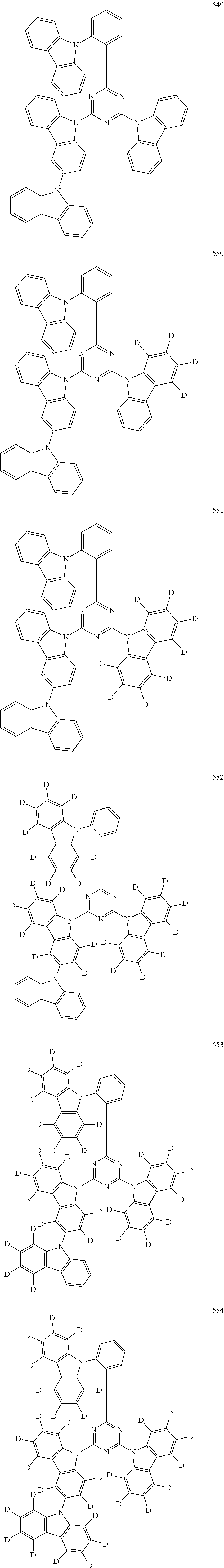

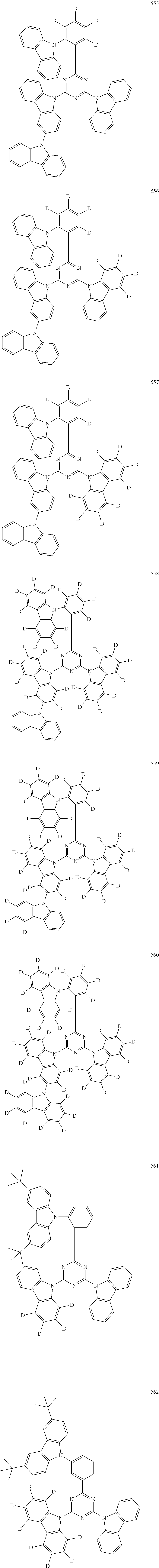

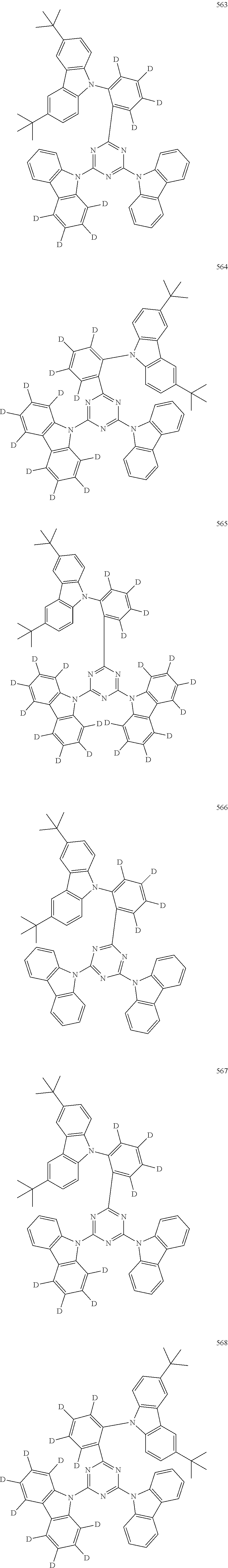

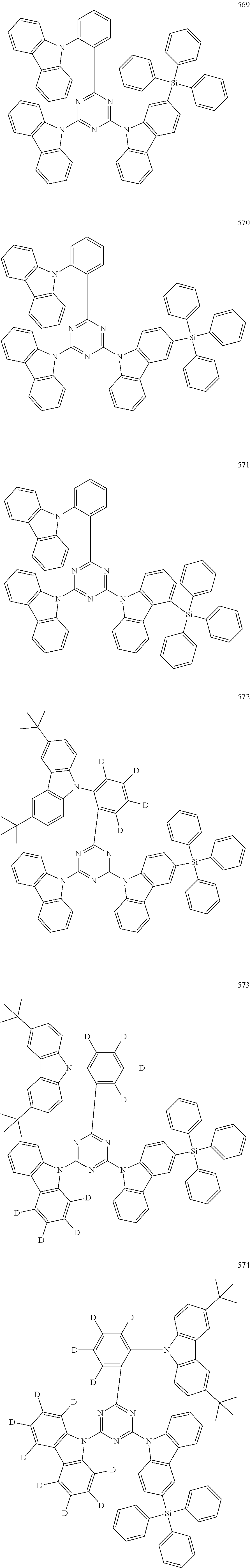

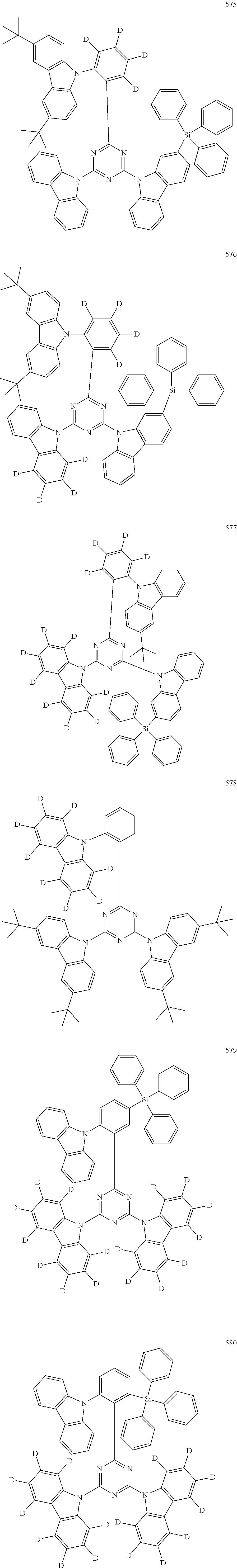

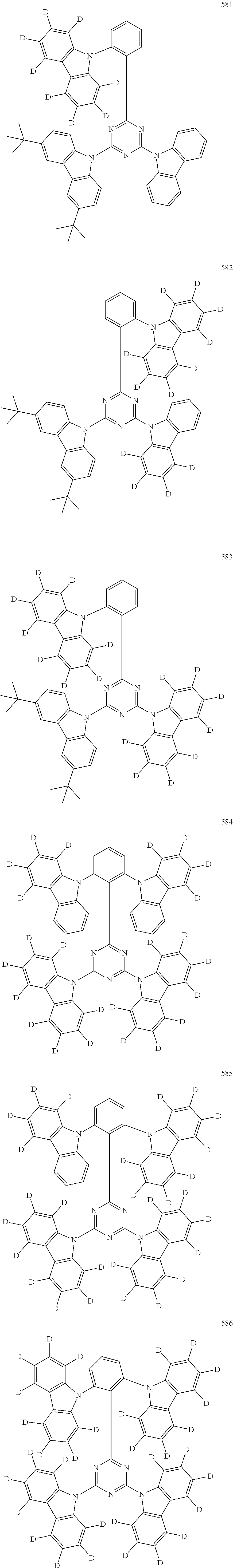

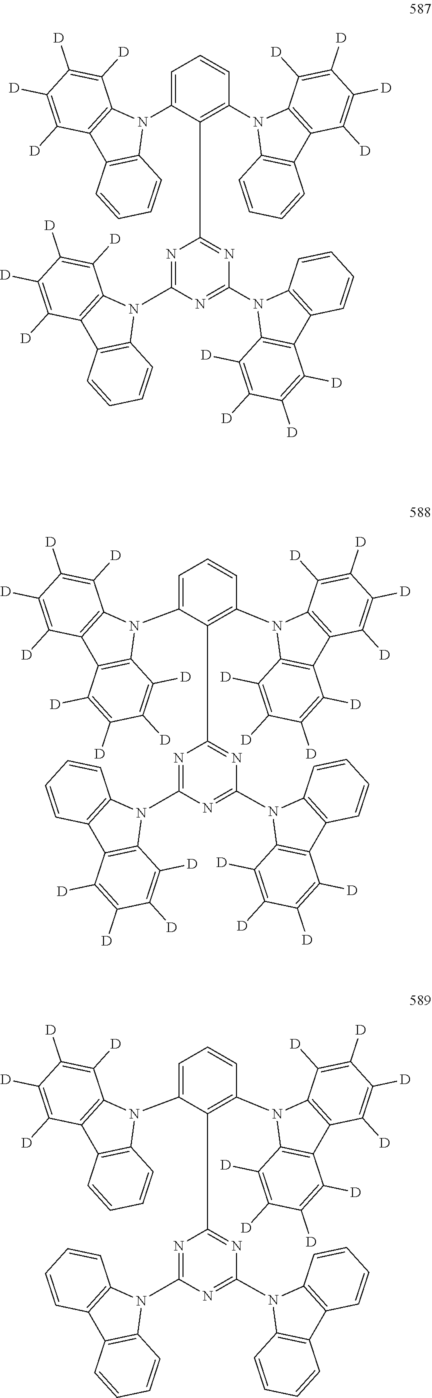

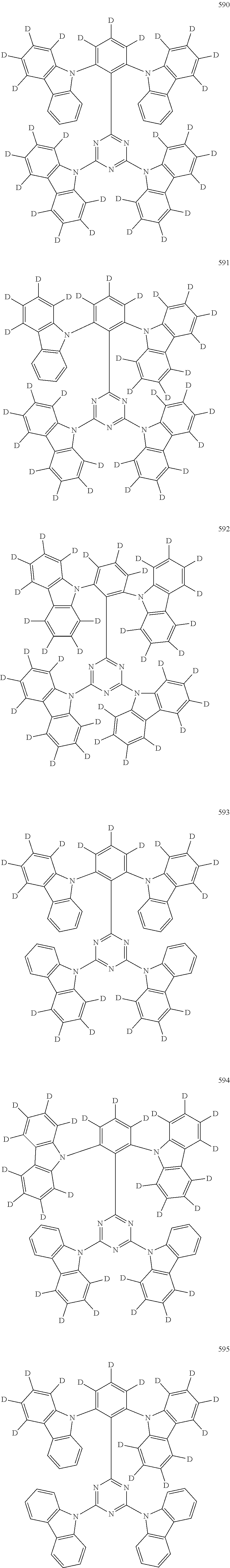

13. A nitrogen-containing compound represented by Formula 1 below: 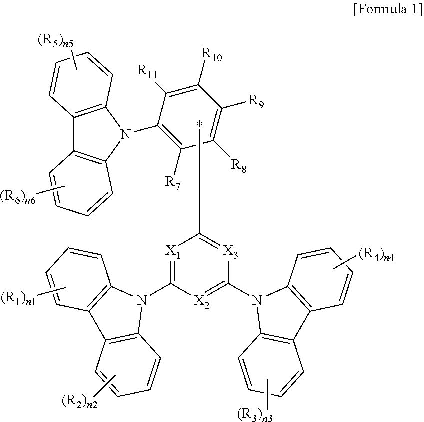
wherein in Formula 1 above,
at least one selected from X₁ to X₃ is N, and the others are each independently CRₓ,
Rₓ is a hydrogen atom, a deuterium atom, a substituted or unsubstituted alkyl group having 1 to 60 carbon atoms, a substituted or unsubstituted aryl group having 6 to 60 ring-forming carbon atoms, or a substituted or unsubstituted heteroaryl group having 2 to 60 ring-forming carbon atoms,
at least one selected from R₁ to R₁₁ is a deuterium atom, an unsubstituted carbazole group, a deuterium atom-substituted or unsubstituted phenyl group, an unsubstituted dibenzofuran group, an unsubstituted dibenzothiophene group, or an unsubstituted 9-phenylcarbazole group, and the others are each independently a hydrogen atom, a cyano group, a substituted or unsubstituted alkyl group having 1 to 60 carbon atoms, a substituted or unsubstituted aryl group having 6 to 60 ring-forming carbon atoms, or a substituted or unsubstituted heteroaryl group having 2 to 60 ring-forming carbon atoms, or bonded to an adjacent group to form a ring,
n1 to n6 are each independently an integer of 0 to 4,
“-*” is connected to any one selected from among R₇ to R₁₁, and
a case where X₁ to X₃ are all N, “-*” is connected to R₇, R₈ is an unsubstituted carbazole group, and R₁ to R₆ and R₉ to R₁₁ are all hydrogen atoms is excluded.

14. The nitrogen-containing compound of claim 13, wherein Formula 1 above is represented by Formula 2 below: 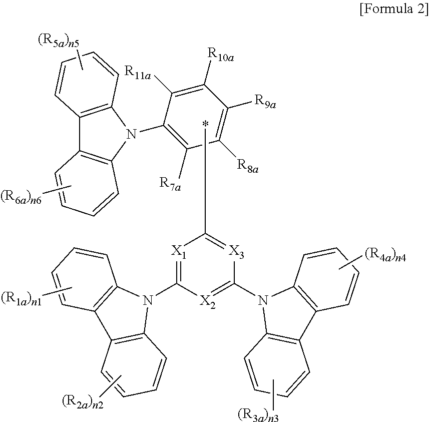
wherein in Formula 2 above,
at least one selected from R₁ₐ to R₁₁ₐ is a deuterium atom, an unsubstituted carbazole group, a deuterium atom-substituted or unsubstituted phenyl group, an unsubstituted dibenzofuran group, an unsubstituted dibenzothiophene group, or an unsubstituted 9-phenylcarbazole group, and the others are hydrogen atoms,
“-*” is connected to any one selected from among R₇ₐ to R₁₁ₐ, and
X₁ to X₃, and n1 to n6 are the same as defined with respect to Formula 1 above.

15. The nitrogen-containing compound of claim 14, wherein Formula 2 above is represented by any one selected from among Formulas 2-a to 2-d below: 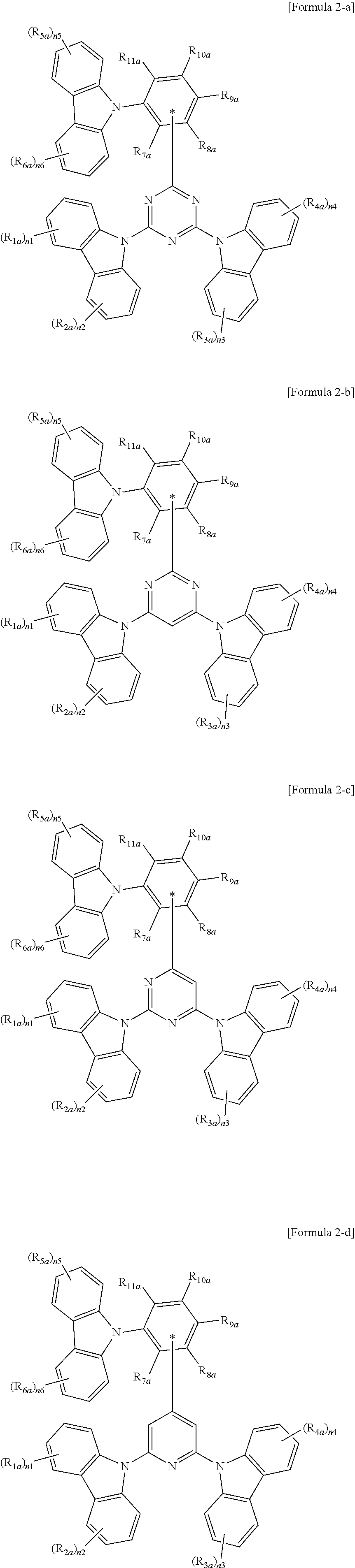
wherein in Formulas 2-a to 2-d above,
R₁ₐ to R₁₁ₐ, n1 to n6, and “-*” are the same as defined with respect to Formula 1 and Formula 2 above.

16. The nitrogen-containing compound of claim 13, wherein Formula 1 above is represented by Formula 3-1 or Formula 3-2 below: 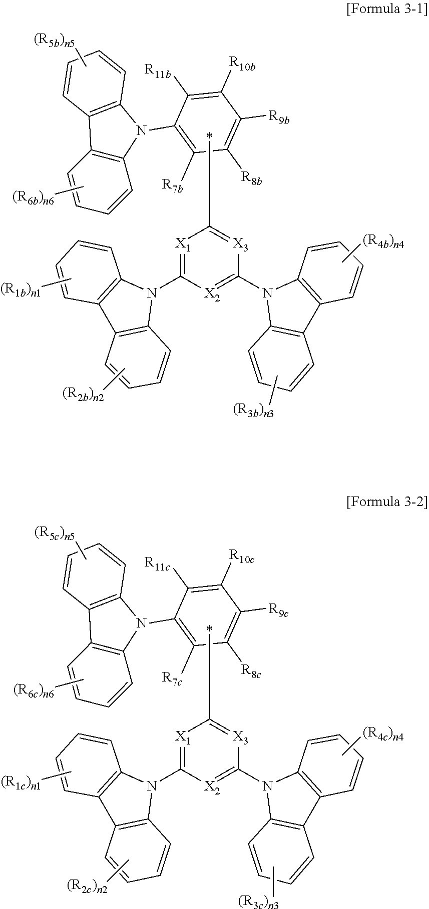
wherein in Formula 3-1 above,
at least one selected from R_{1b} to R_{11b} is a deuterium atom, and the others are each independently a hydrogen atom, a cyano group, a substituted or unsubstituted alkyl group having 1 to 60 carbon atoms, a substituted or unsubstituted aryl group having 6 to 60 ring-forming carbon atoms, or a substituted or unsubstituted heteroaryl group having 2 to 60 ring-forming carbon atoms, or bonded to an adjacent group to form a ring, and
“-*” is connected to any one selected from among R_{7b} to R_{11b},
wherein in Formula 3-2 above,
R_{1c} to R_{6c} are each independently a hydrogen atom, a deuterium atom, a cyano group, a substituted or unsubstituted alkyl group having 1 to 60 carbon atoms, a substituted or unsubstituted aryl group having 6 to 60 ring-forming carbon atoms, or a substituted or unsubstituted heteroaryl group having 2 to 60 ring-forming carbon atoms, or bonded to an adjacent group to form a ring,
at least one selected from R_{7c} to R_{11c} is an unsubstituted carbazole group, a deuterium atom-substituted or unsubstituted phenyl group, an unsubstituted dibenzofuran group, an unsubstituted dibenzothiophene group, or an unsubstituted 9-phenylcarbazole group, and the others are each independently a hydrogen atom or a deuterium atom, and
“-*” is connected to any one selected from among R_{7c} to R_{11c}, and
wherein in Formulas 3-1 and 3-2 above,
X₁ to X₃, and n1 to n6 are the same as defined with respect to Formula 1 above.

17. The nitrogen-containing compound of claim 16, wherein at least one selected from R_{1b} to R_{11b} is a deuterium atom, and the others are hydrogen atoms.

18. The nitrogen-containing compound of claim 16, wherein Formula 3-2 above is represented by any one selected from among Formulas 3-2a to 3-2c below: 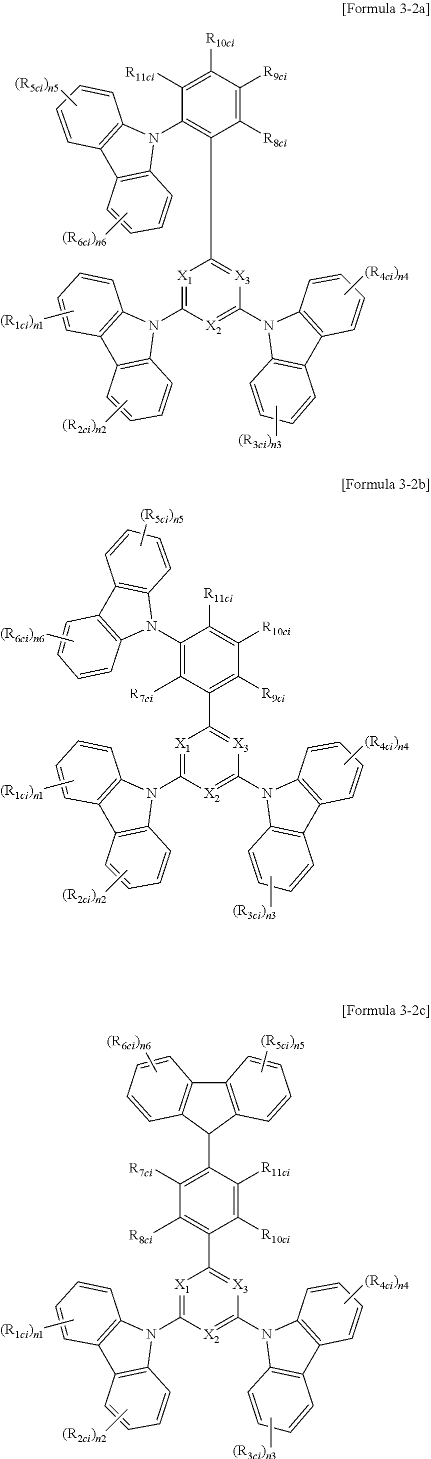
wherein in Formula 3-2a above,
any one among R_{8ci} to R_{11ci} is an unsubstituted carbazole group, a deuterium atom-substituted or unsubstituted phenyl group, an unsubstituted dibenzofuran group, an unsubstituted dibenzothiophene group, or an unsubstituted 9-phenylcarbazole group, and the others are each independently a hydrogen atom or a deuterium atom,
wherein in Formula 3-2b above,
any one among R_{7ci}, and R_{9ci} to R_{11ci} is an unsubstituted carbazole group, a deuterium atom-substituted or unsubstituted phenyl group, an unsubstituted dibenzofuran group, an unsubstituted dibenzothiophene group, or an unsubstituted 9-phenylcarbazole group, and the others are each independently a hydrogen atom or a deuterium atom,
wherein in Formula 3-2c above,
any one among R_{7ci}, R_{8ci}, R_{10ci}, and R_{11ci} is an unsubstituted carbazole group, a deuterium atom-substituted or unsubstituted phenyl group, an unsubstituted dibenzofuran group, an unsubstituted dibenzothiophene group, or an unsubstituted 9-phenylcarbazole group, and the others are each independently a hydrogen atom or a deuterium atom, and
wherein in Formulas 3-2a to 3-2c above,
R_{1ci} to R_{6ci} are each independently a hydrogen atom or a deuterium atom, and
X₁ to X₃, and n1 to n6 are the same as defined with respect to Formula 3-2 above.

19. The nitrogen-containing compound of claim 13, wherein Formula 1 above is represented by Formula 4 below: 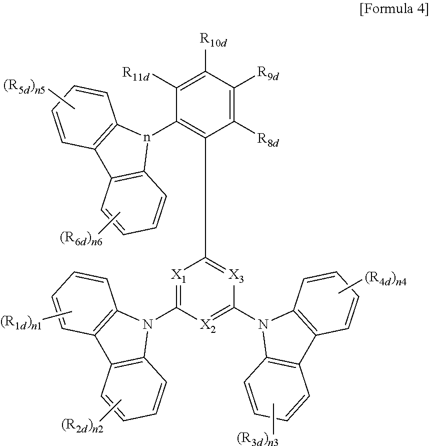
wherein in Formula 4 above,
R_{1d} to R_{6d} are each independently a hydrogen atom or a deuterium atom,
any one among R_{8d} to R_{11d} is a deuterium atom, an unsubstituted carbazole group, a deuterium atom-substituted or unsubstituted phenyl group, an unsubstituted dibenzofuran group, an unsubstituted dibenzothiophene group, or an unsubstituted 9-phenylcarbazole group, and the others are each independently a hydrogen atom or a deuterium atom, and
X₁ to X₃, and n1 to n6 are the same as defined with respect to Formula 1 above.

20. The nitrogen-containing compound of claim 13, wherein Formula 1 above is represented by any one selected from among compounds of Compound Group 1 below: 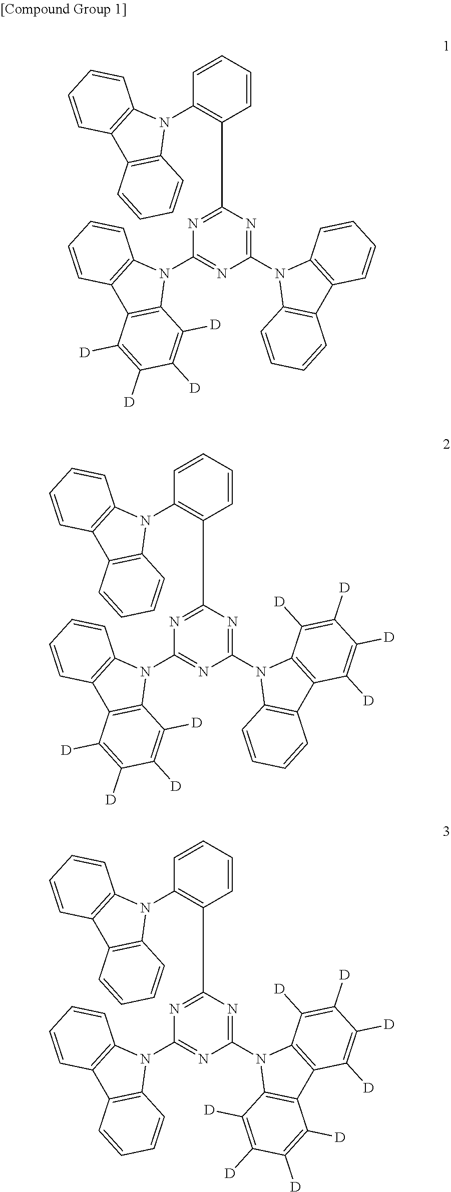 [SMILES: [2H]c1c([2H])c([2H])c2c(c1[2H])c1ccccc1n2-c1nc(-c2ccccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2ccccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1c([2H])c([2H])c([2H])c([2H])c1n2-c1nc(-c2ccccc2-n2c3ccccc3c3ccccc32)nc(-n2c3ccccc3c3ccccc32)n1] 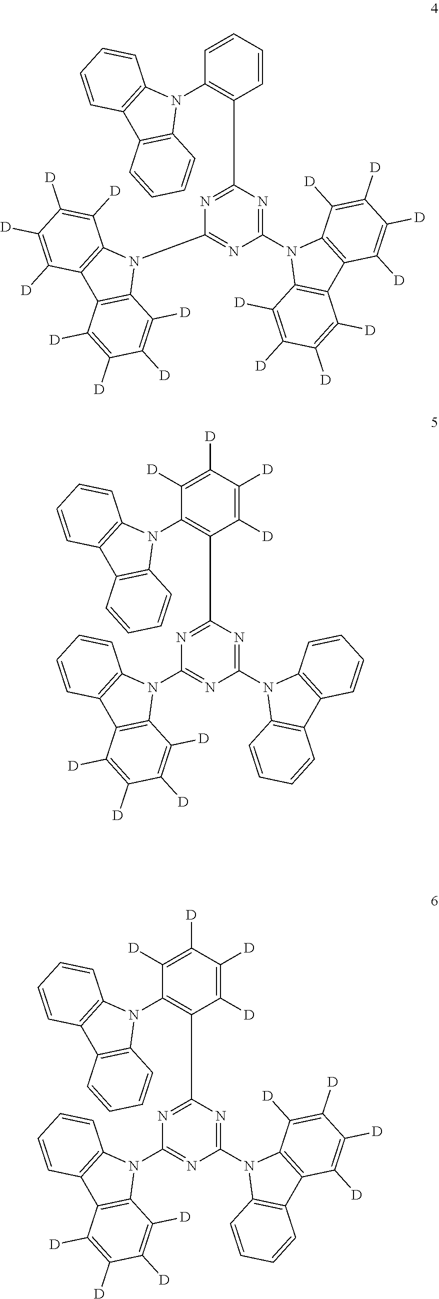 [SMILES: [2H]c1c([2H])c([2H])c2c(c1[2H])c1c([2H])c([2H])c([2H])c([2H])c1n2-c1nc(-c2ccccc2-n2c3ccccc3c3ccccc32)nc(-n2c3c([2H])c([2H])c([2H])c([2H])c3c3c([2H])c([2H])c([2H])c([2H])c32)n1 ; [2H]c1c([2H])c([2H])c(-n2c3ccccc3c3ccccc32)c(-c2nc(-n3c4ccccc4c4ccccc43)nc(-n3c4ccccc4c4c([2H])c([2H])c([2H])c([2H])c43)n2)c1[2H] ; [2H]c1c([2H])c([2H])c(-n2c3ccccc3c3ccccc32)c(-c2nc(-n3c4ccccc4c4c([2H])c([2H])c([2H])c([2H])c43)nc(-n3c4ccccc4c4c([2H])c([2H])c([2H])c([2H])c43)n2)c1[2H]] 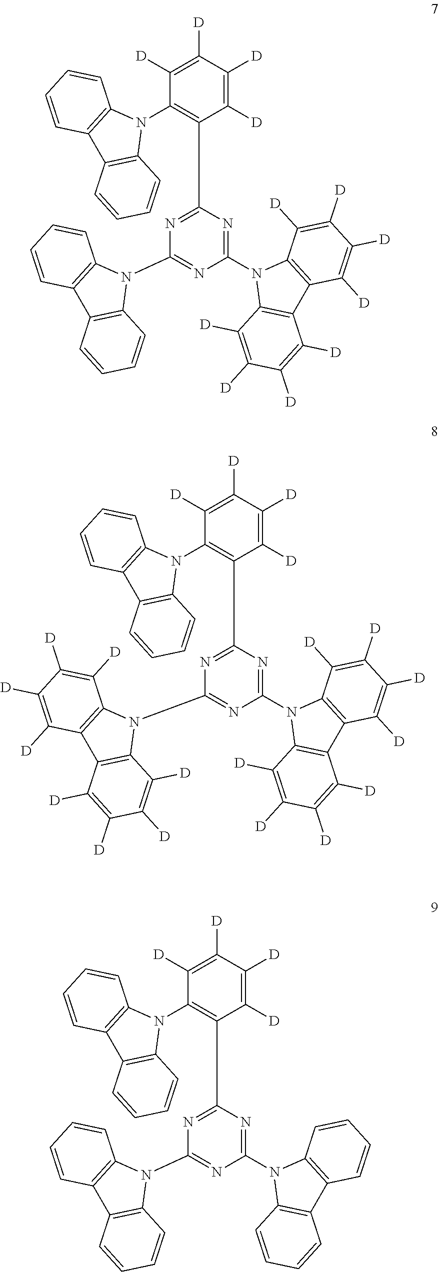 [SMILES: [2H]c1c([2H])c([2H])c(-n2c3ccccc3c3ccccc32)c(-c2nc(-n3c4ccccc4c4ccccc43)nc(-n3c4c([2H])c([2H])c([2H])c([2H])c4c4c([2H])c([2H])c([2H])c([2H])c43)n2)c1[2H] ; [2H]c1c([2H])c([2H])c(-n2c3ccccc3c3ccccc32)c(-c2nc(-n3c4c([2H])c([2H])c([2H])c([2H])c4c4c([2H])c([2H])c([2H])c([2H])c43)nc(-n3c4c([2H])c([2H])c([2H])c([2H])c4c4c([2H])c([2H])c([2H])c([2H])c43)n2)c1[2H] ; [2H]c1c([2H])c([2H])c(-n2c3ccccc3c3ccccc32)c(-c2nc(-n3c4ccccc4c4ccccc43)nc(-n3c4ccccc4c4ccccc43)n2)c1[2H]] 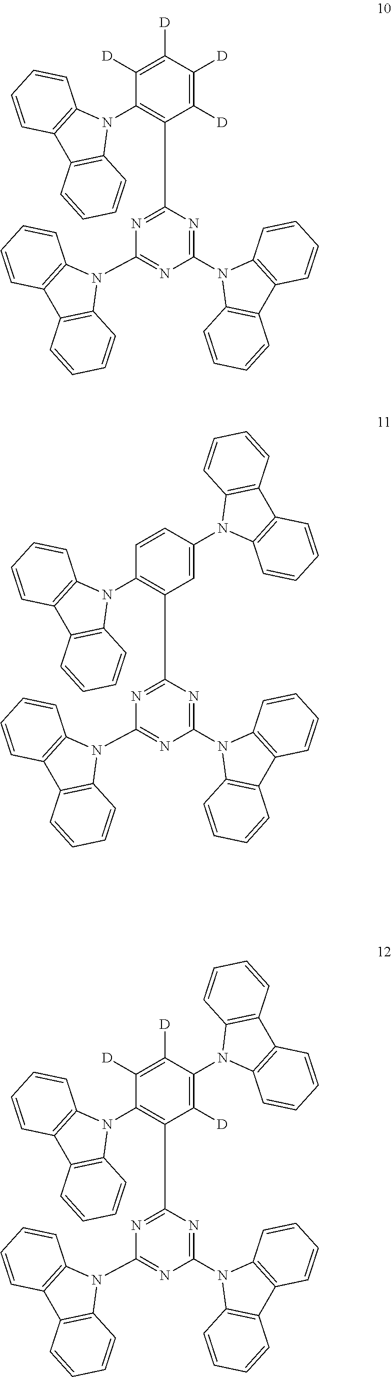 [SMILES: [2H]c1c([2H])c([2H])c(-n2c3ccccc3c3ccccc32)c(-c2nc(-n3c4ccccc4c4ccccc43)nc(-n3c4ccccc4c4ccccc43)n2)c1[2H] ; c1ccc2c(c1)c1ccccc1n2-c1ccc(-n2c3ccccc3c3ccccc32)c(-c2nc(-n3c4ccccc4c4ccccc43)nc(-n3c4ccccc4c4ccccc43)n2)c1 ; [2H]c1c([2H])c(-n2c3ccccc3c3ccccc32)c(-c2nc(-n3c4ccccc4c4ccccc43)nc(-n3c4ccccc4c4ccccc43)n2)c([2H])c1-n1c2ccccc2c2ccccc21] 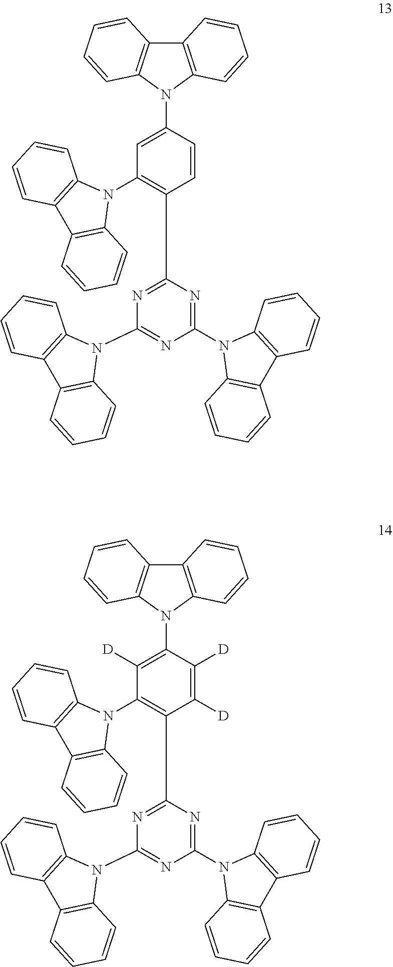 [SMILES: c1ccc2c(c1)c1ccccc1n2-c1ccc(-c2nc(-n3c4ccccc4c4ccccc43)nc(-n3c4ccccc4c4ccccc43)n2)c(-n2c3ccccc3c3ccccc32)c1 ; [2H]c1c([2H])c(-n2c3ccccc3c3ccccc32)c([2H])c(-n2c3ccccc3c3ccccc32)c1-c1nc(-n2c3ccccc3c3ccccc32)nc(-n2c3ccccc3c3ccccc32)n1] 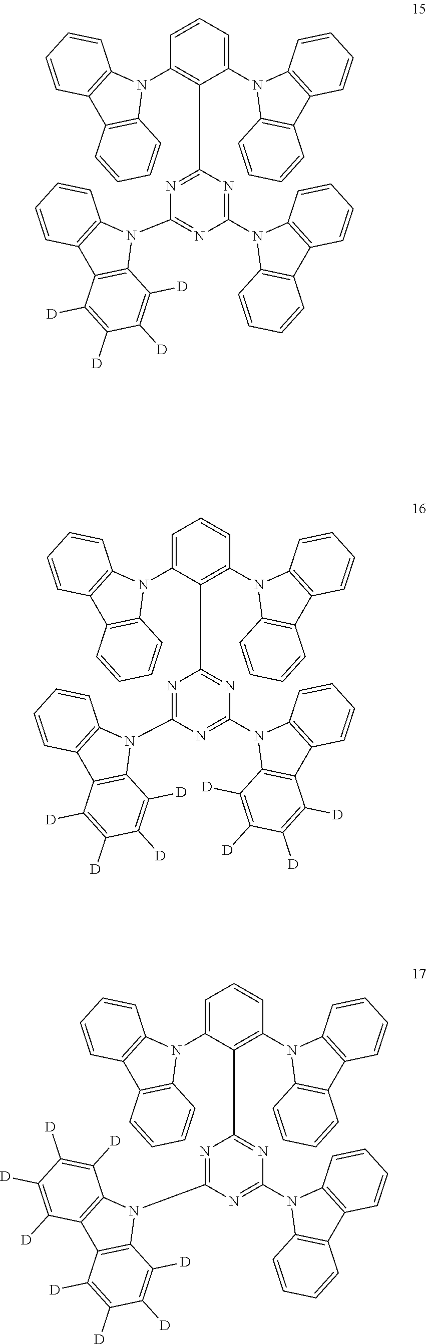 [SMILES: [2H]c1c([2H])c([2H])c2c(c1[2H])c1ccccc1n2-c1nc(-c2c(-n3c4ccccc4c4ccccc43)cccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2c(-n3c4ccccc4c4ccccc43)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1c([2H])c([2H])c([2H])c([2H])c1n2-c1nc(-c2c(-n3c4ccccc4c4ccccc43)cccc2-n2c3ccccc3c3ccccc32)nc(-n2c3ccccc3c3ccccc32)n1] 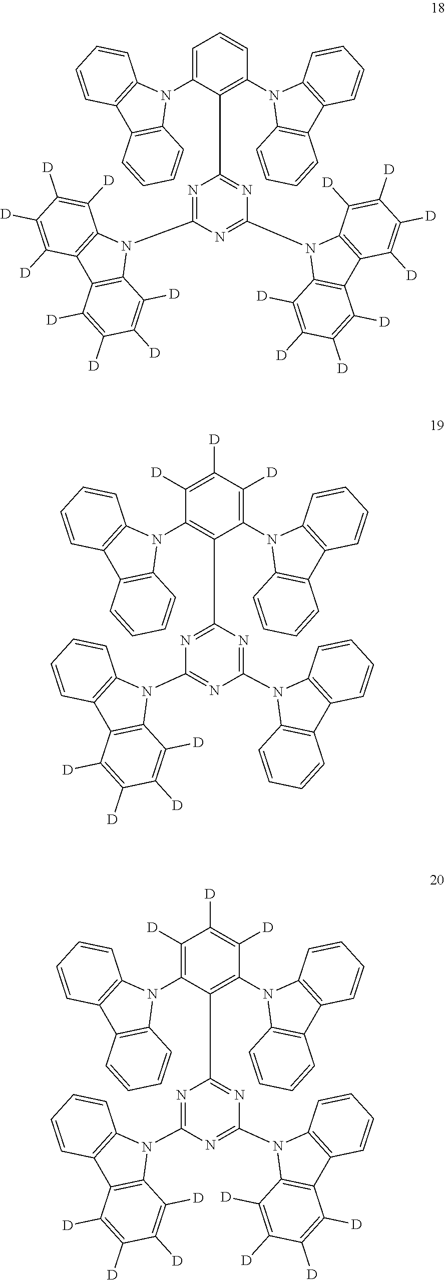 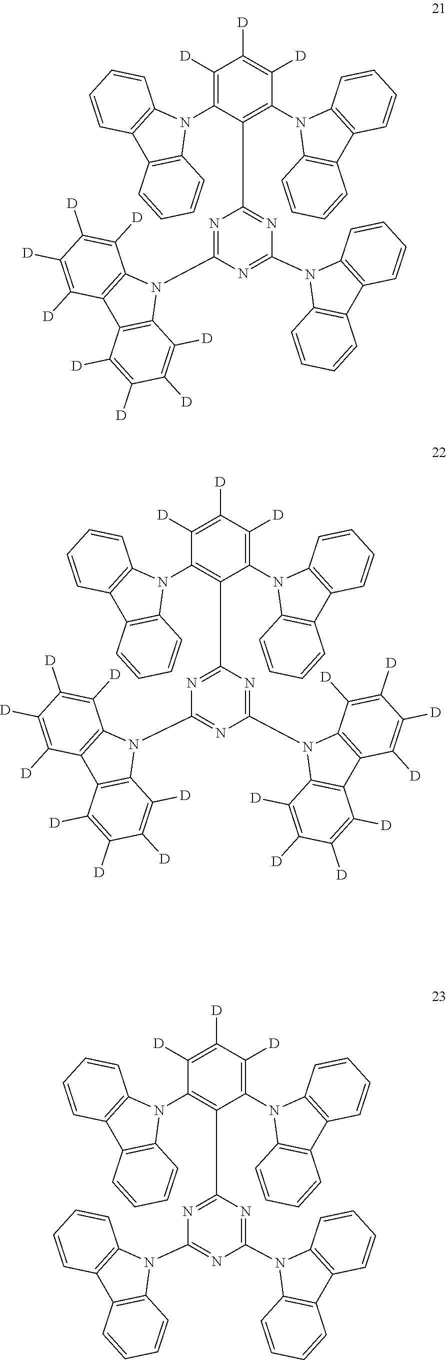 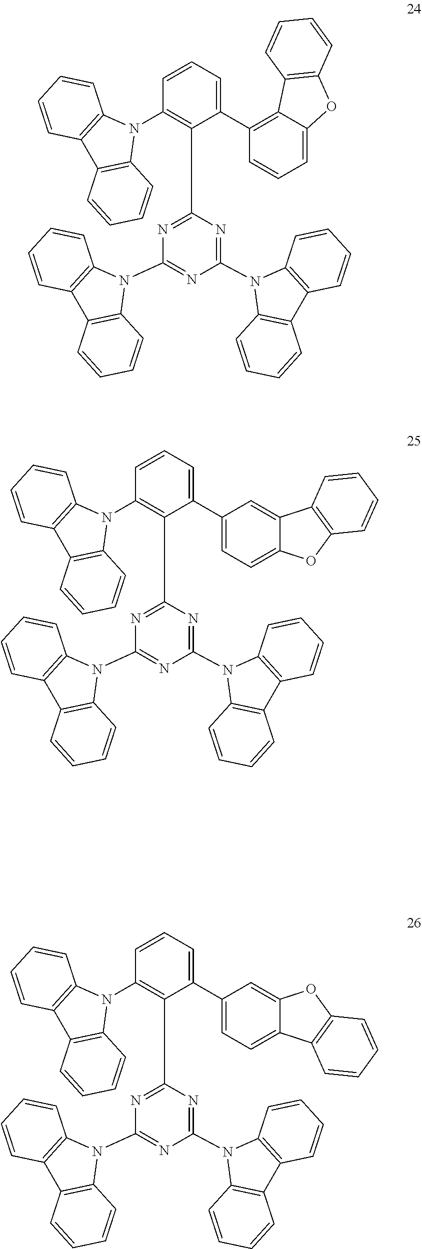 [SMILES: c1cc(-c2cccc3oc4ccccc4c23)c(-c2nc(-n3c4ccccc4c4ccccc43)nc(-n3c4ccccc4c4ccccc43)n2)c(-n2c3ccccc3c3ccccc32)c1 ; c1cc(-c2ccc3oc4ccccc4c3c2)c(-c2nc(-n3c4ccccc4c4ccccc43)nc(-n3c4ccccc4c4ccccc43)n2)c(-n2c3ccccc3c3ccccc32)c1 ; c1cc(-c2ccc3c(c2)oc2ccccc23)c(-c2nc(-n3c4ccccc4c4ccccc43)nc(-n3c4ccccc4c4ccccc43)n2)c(-n2c3ccccc3c3ccccc32)c1] 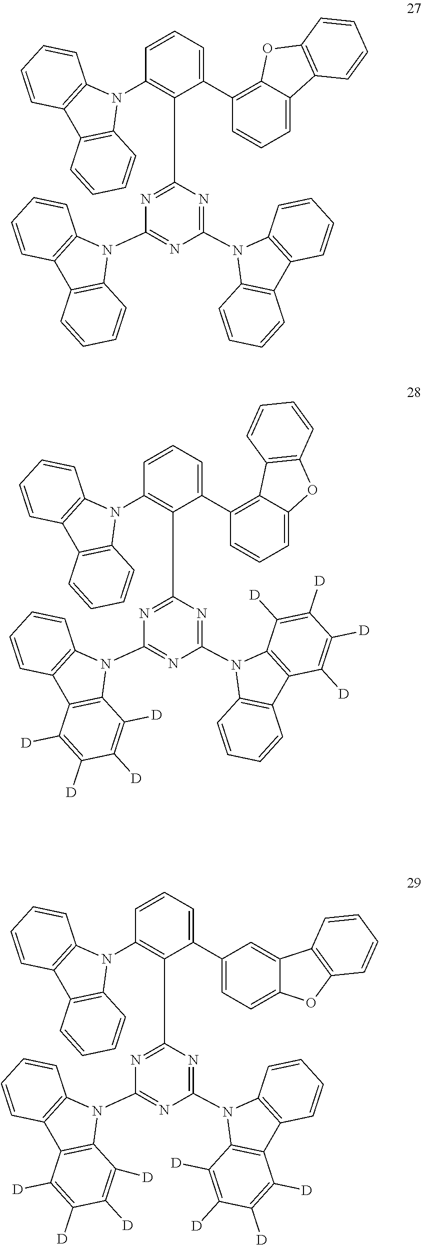 [SMILES: c1cc(-c2cccc3c2oc2ccccc23)c(-c2nc(-n3c4ccccc4c4ccccc43)nc(-n3c4ccccc4c4ccccc43)n2)c(-n2c3ccccc3c3ccccc32)c1 ; [2H]c1c([2H])c([2H])c2c(c1[2H])c1ccccc1n2-c1nc(-c2c(-c3cccc4oc5ccccc5c34)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2c(-c3ccc4oc5ccccc5c4c3)cccc2-n2c3ccccc3c3ccccc32)nc(-n2c3ccccc3c3c([2H])c([2H])c([2H])c([2H])c32)n1] 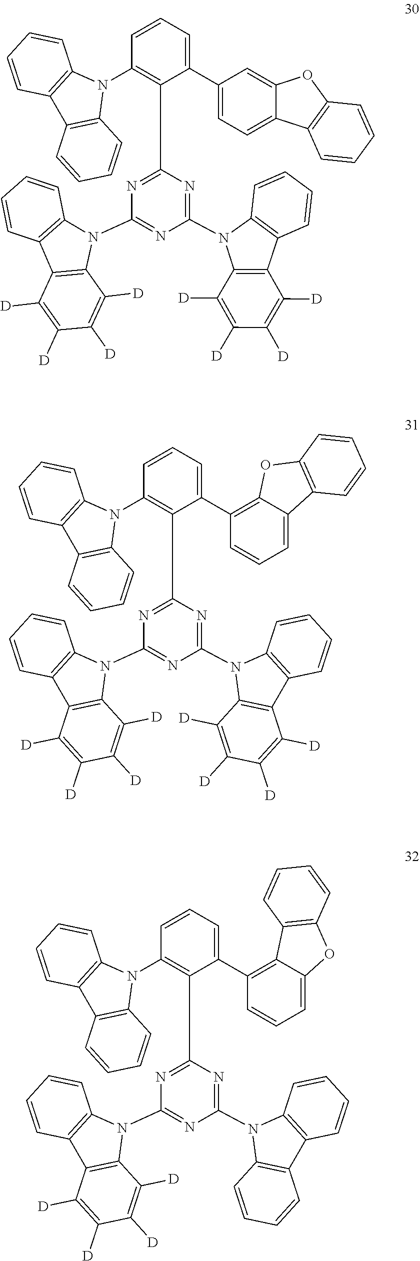 [SMILES: [2H]c1c([2H])c([2H])c2c(c1[2H])c1ccccc1n2-c1nc(-c2c(-c3ccc4c(c3)oc3ccccc34)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2c(-c3cccc4c3oc3ccccc34)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2c(-c3cccc4oc5ccccc5c34)cccc2-n2c3ccccc3c3ccccc32)nc(-n2c3ccccc3c3ccccc32)n1] 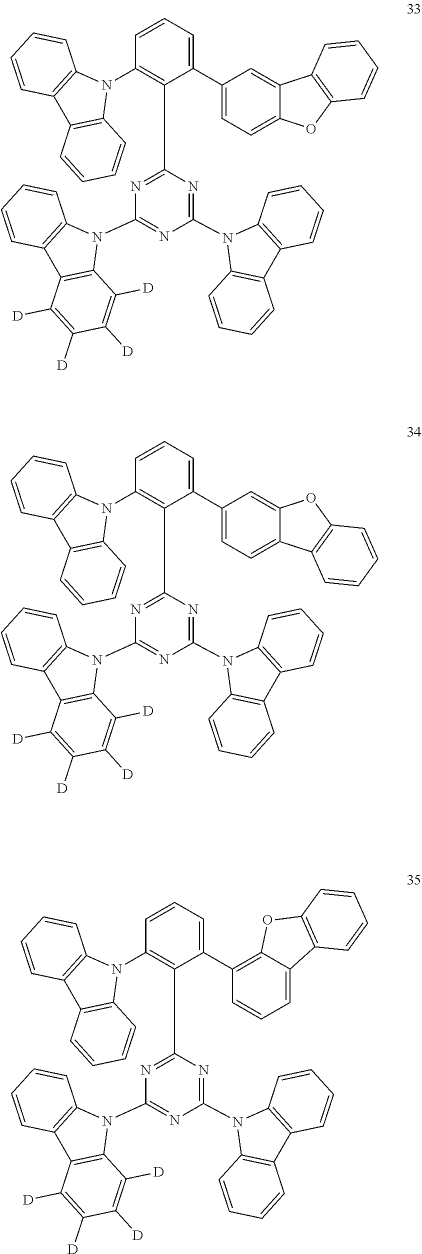 [SMILES: [2H]c1c([2H])c([2H])c2c(c1[2H])c1ccccc1n2-c1nc(-c2c(-c3ccc4oc5ccccc5c4c3)cccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2c(-c3ccc4c(c3)oc3ccccc34)cccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2c(-c3cccc4c3oc3ccccc34)cccc2-n2c3ccccc3c3ccccc32)nc(-n2c3ccccc3c3ccccc32)n1] 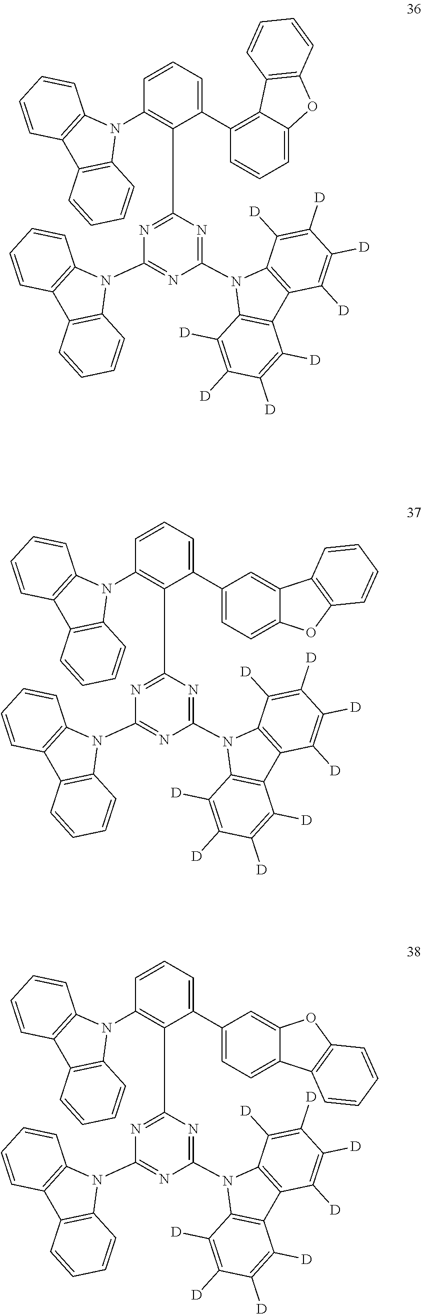 [SMILES: [2H]c1c([2H])c([2H])c2c(c1[2H])c1c([2H])c([2H])c([2H])c([2H])c1n2-c1nc(-c2c(-c3cccc4oc5ccccc5c34)cccc2-n2c3ccccc3c3ccccc32)nc(-n2c3ccccc3c3ccccc32)n1 ; [2H]c1c([2H])c([2H])c2c(c1[2H])c1c([2H])c([2H])c([2H])c([2H])c1n2-c1nc(-c2c(-c3ccc4oc5ccccc5c4c3)cccc2-n2c3ccccc3c3ccccc32)nc(-n2c3ccccc3c3ccccc32)n1 ; [2H]c1c([2H])c([2H])c2c(c1[2H])c1c([2H])c([2H])c([2H])c([2H])c1n2-c1nc(-c2c(-c3ccc4c(c3)oc3ccccc34)cccc2-n2c3ccccc3c3ccccc32)nc(-n2c3ccccc3c3ccccc32)n1] 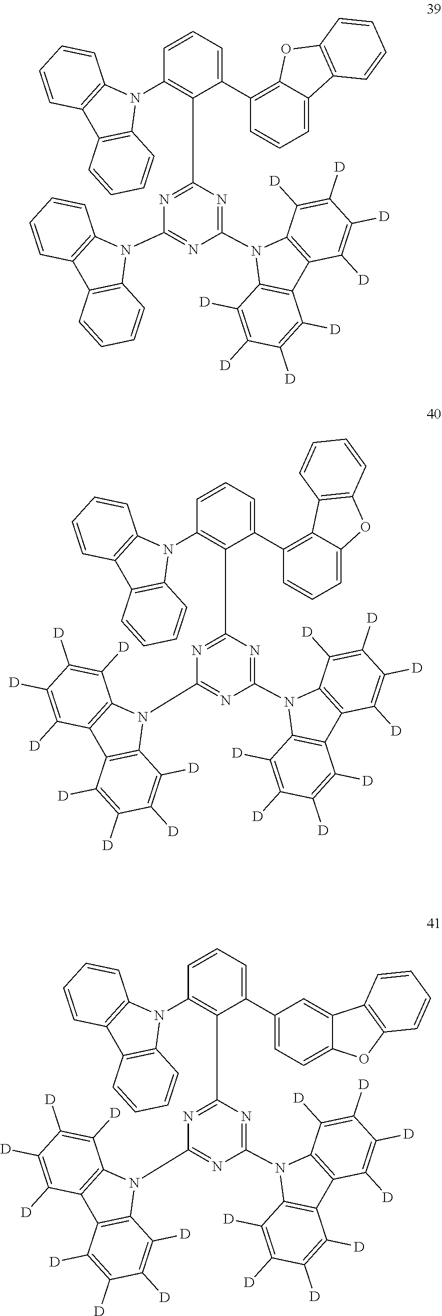 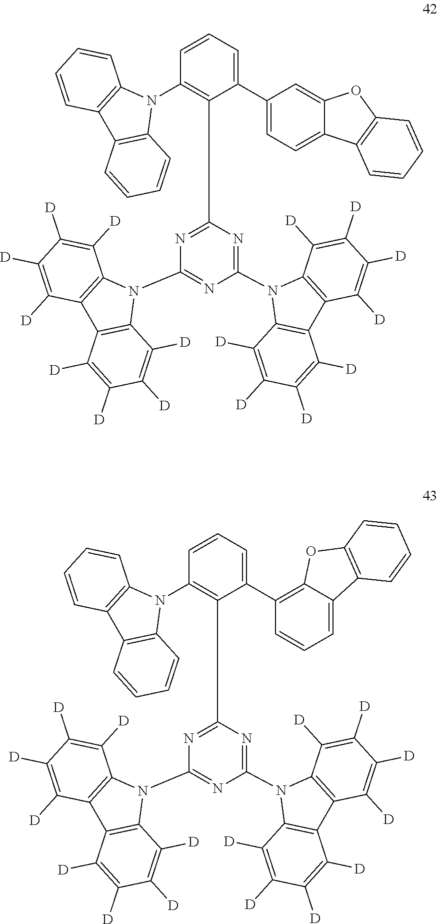 [SMILES: [2H]c1c([2H])c([2H])c2c(c1[2H])c1c([2H])c([2H])c([2H])c([2H])c1n2-c1nc(-c2c(-c3ccc4c(c3)oc3ccccc34)cccc2-n2c3ccccc3c3ccccc32)nc(-n2c3c([2H])c([2H])c([2H])c([2H])c3c3c([2H])c([2H])c([2H])c([2H])c32)n1 ; [2H]c1c([2H])c([2H])c2c(c1[2H])c1c([2H])c([2H])c([2H])c([2H])c1n2-c1nc(-c2c(-c3cccc4c3oc3ccccc34)cccc2-n2c3ccccc3c3ccccc32)nc(-n2c3c([2H])c([2H])c([2H])c([2H])c3c3c([2H])c([2H])c([2H])c([2H])c32)n1] 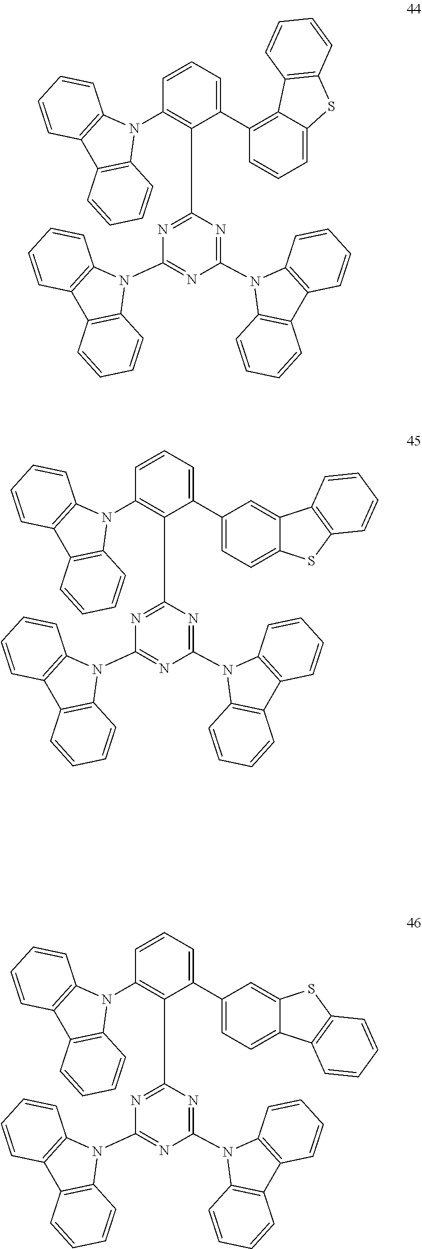 [SMILES: c1cc(-c2cccc3sc4ccccc4c23)c(-c2nc(-n3c4ccccc4c4ccccc43)nc(-n3c4ccccc4c4ccccc43)n2)c(-n2c3ccccc3c3ccccc32)c1 ; c1cc(-c2ccc3sc4ccccc4c3c2)c(-c2nc(-n3c4ccccc4c4ccccc43)nc(-n3c4ccccc4c4ccccc43)n2)c(-n2c3ccccc3c3ccccc32)c1 ; c1cc(-c2ccc3c(c2)sc2ccccc23)c(-c2nc(-n3c4ccccc4c4ccccc43)nc(-n3c4ccccc4c4ccccc43)n2)c(-n2c3ccccc3c3ccccc32)c1] 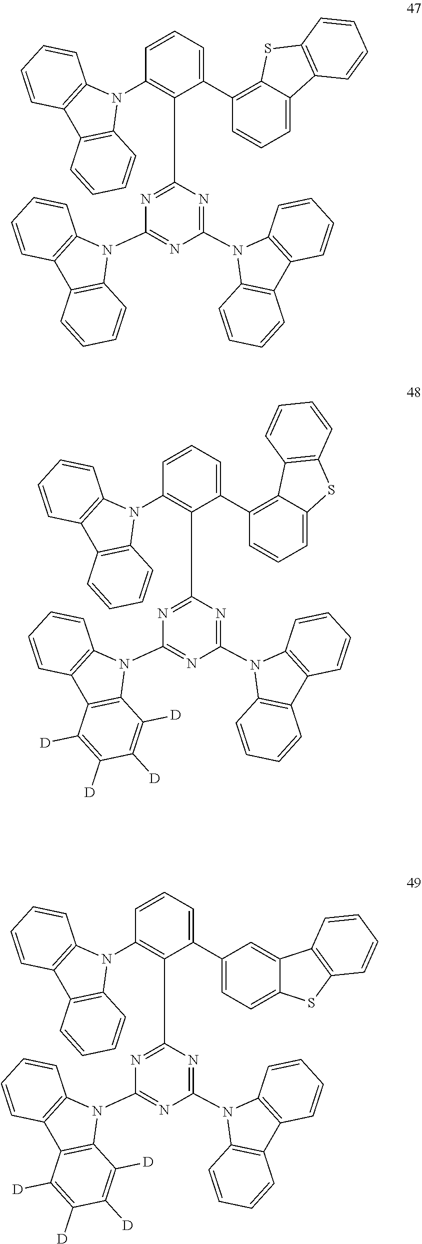 [SMILES: c1cc(-c2cccc3c2sc2ccccc23)c(-c2nc(-n3c4ccccc4c4ccccc43)nc(-n3c4ccccc4c4ccccc43)n2)c(-n2c3ccccc3c3ccccc32)c1 ; [2H]c1c([2H])c([2H])c2c(c1[2H])c1ccccc1n2-c1nc(-c2c(-c3cccc4sc5ccccc5c34)cccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2c(-c3ccc4sc5ccccc5c4c3)cccc2-n2c3ccccc3c3ccccc32)nc(-n2c3ccccc3c3ccccc32)n1] 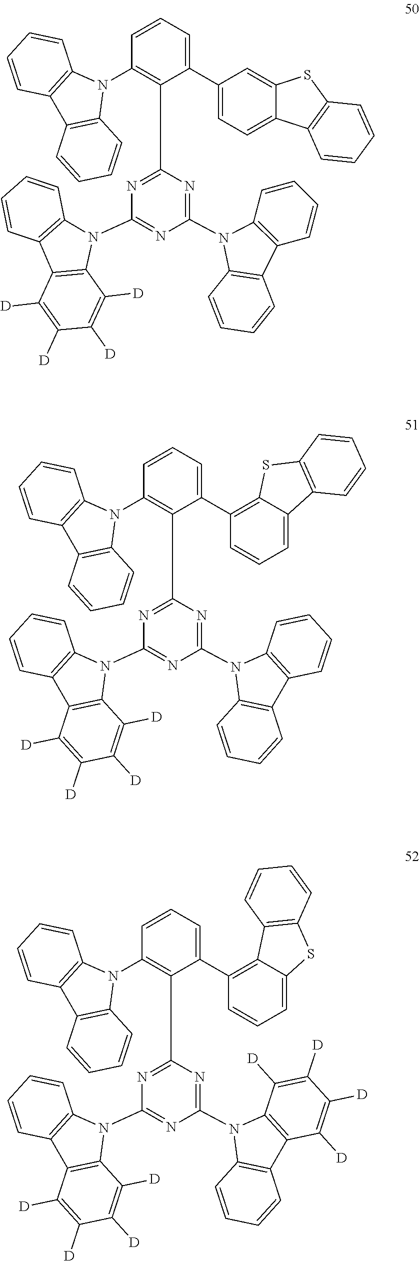 [SMILES: [2H]c1c([2H])c([2H])c2c(c1[2H])c1ccccc1n2-c1nc(-c2c(-c3ccc4c(c3)sc3ccccc34)cccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2c(-c3cccc4c3sc3ccccc34)cccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2c(-c3cccc4sc5ccccc5c34)cccc2-n2c3ccccc3c3ccccc32)nc(-n2c3ccccc3c3c([2H])c([2H])c([2H])c([2H])c32)n1] 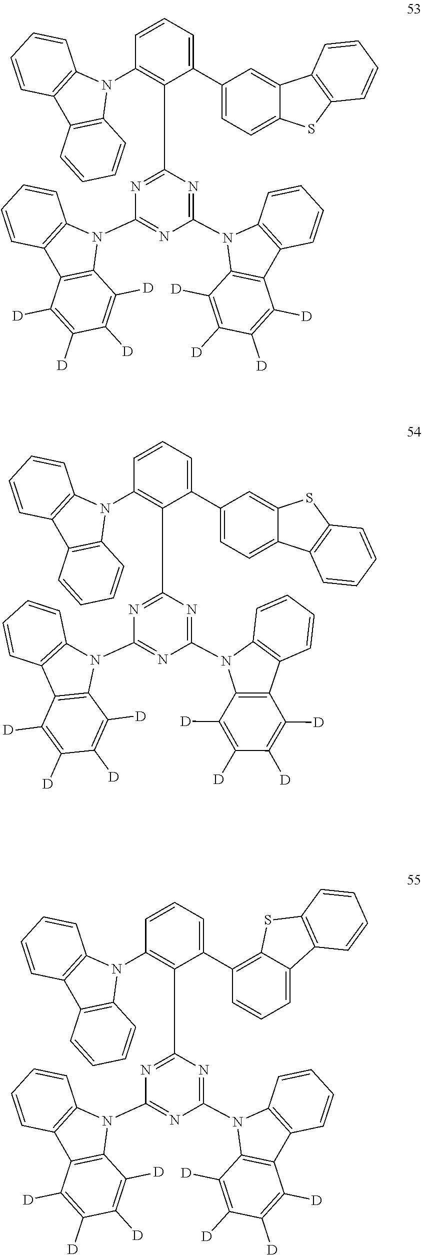 [SMILES: [2H]c1c([2H])c([2H])c2c(c1[2H])c1ccccc1n2-c1nc(-c2c(-c3ccc4sc5ccccc5c4c3)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2c(-c3ccc4c(c3)sc3ccccc34)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2c(-c3cccc4c3sc3ccccc34)cccc2-n2c3ccccc3c3ccccc32)nc(-n2c3ccccc3c3c([2H])c([2H])c([2H])c([2H])c32)n1] 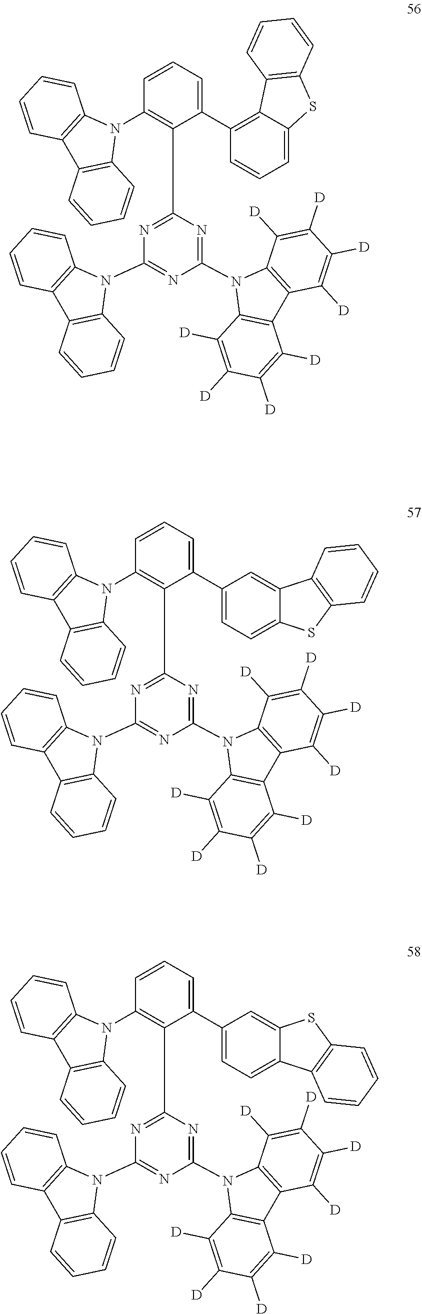 [SMILES: [2H]c1c([2H])c([2H])c2c(c1[2H])c1c([2H])c([2H])c([2H])c([2H])c1n2-c1nc(-c2c(-c3cccc4sc5ccccc5c34)cccc2-n2c3ccccc3c3ccccc32)nc(-n2c3ccccc3c3ccccc32)n1 ; [2H]c1c([2H])c([2H])c2c(c1[2H])c1c([2H])c([2H])c([2H])c([2H])c1n2-c1nc(-c2c(-c3ccc4sc5ccccc5c4c3)cccc2-n2c3ccccc3c3ccccc32)nc(-n2c3ccccc3c3ccccc32)n1 ; [2H]c1c([2H])c([2H])c2c(c1[2H])c1c([2H])c([2H])c([2H])c([2H])c1n2-c1nc(-c2c(-c3ccc4c(c3)sc3ccccc34)cccc2-n2c3ccccc3c3ccccc32)nc(-n2c3ccccc3c3ccccc32)n1] 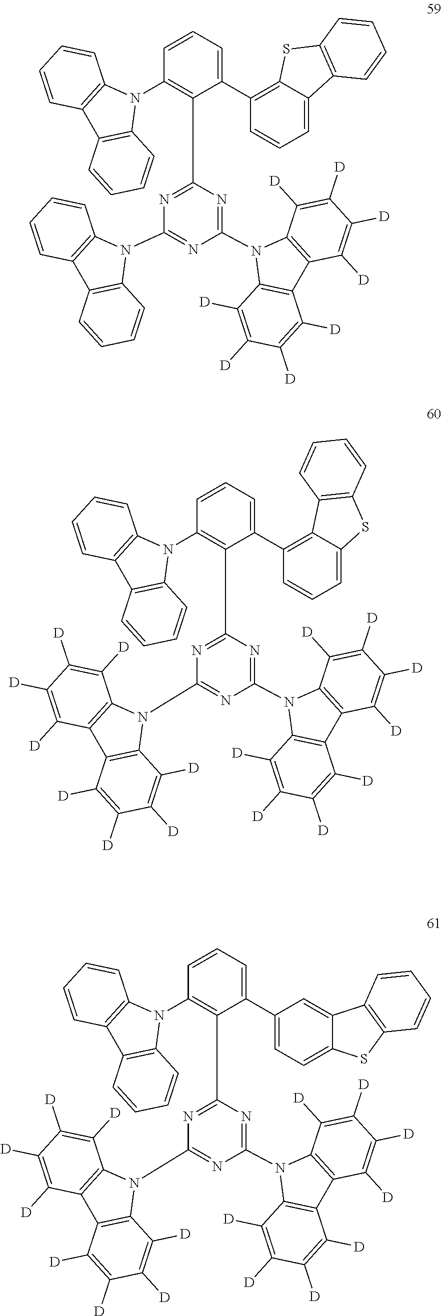 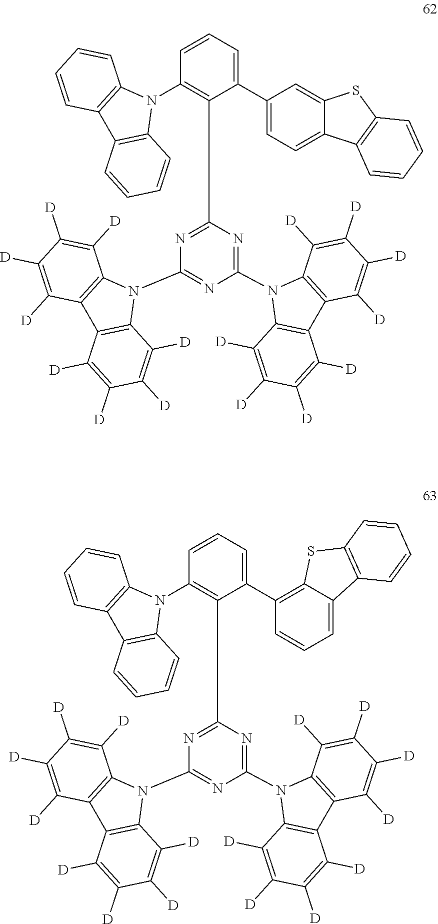 [SMILES: [2H]c1c([2H])c([2H])c2c(c1[2H])c1c([2H])c([2H])c([2H])c([2H])c1n2-c1nc(-c2c(-c3ccc4c(c3)sc3ccccc34)cccc2-n2c3ccccc3c3ccccc32)nc(-n2c3c([2H])c([2H])c([2H])c([2H])c3c3c([2H])c([2H])c([2H])c([2H])c32)n1 ; [2H]c1c([2H])c([2H])c2c(c1[2H])c1c([2H])c([2H])c([2H])c([2H])c1n2-c1nc(-c2c(-c3cccc4c3sc3ccccc34)cccc2-n2c3ccccc3c3ccccc32)nc(-n2c3c([2H])c([2H])c([2H])c([2H])c3c3c([2H])c([2H])c([2H])c([2H])c32)n1] 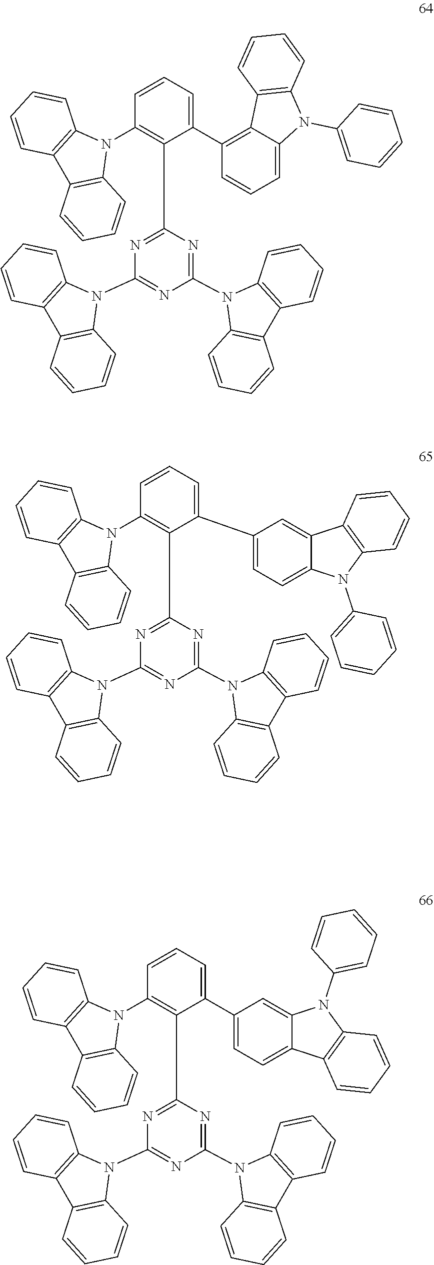 [SMILES: c1ccc(-n2c3ccccc3c3c(-c4cccc(-n5c6ccccc6c6ccccc65)c4-c4nc(-n5c6ccccc6c6ccccc65)nc(-n5c6ccccc6c6ccccc65)n4)cccc32)cc1 ; c1ccc(-n2c3ccccc3c3cc(-c4cccc(-n5c6ccccc6c6ccccc65)c4-c4nc(-n5c6ccccc6c6ccccc65)nc(-n5c6ccccc6c6ccccc65)n4)ccc32)cc1 ; c1ccc(-n2c3ccccc3c3ccc(-c4cccc(-n5c6ccccc6c6ccccc65)c4-c4nc(-n5c6ccccc6c6ccccc65)nc(-n5c6ccccc6c6ccccc65)n4)cc32)cc1] 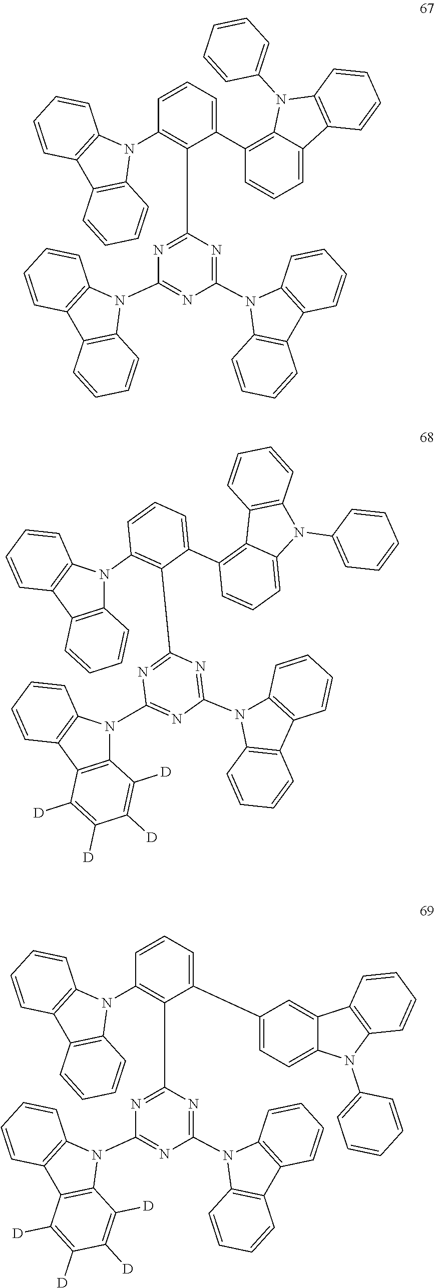 [SMILES: c1ccc(-n2c3ccccc3c3cccc(-c4cccc(-n5c6ccccc6c6ccccc65)c4-c4nc(-n5c6ccccc6c6ccccc65)nc(-n5c6ccccc6c6ccccc65)n4)c32)cc1 ; [2H]c1c([2H])c([2H])c2c(c1[2H])c1ccccc1n2-c1nc(-c2c(-c3cccc4c3c3ccccc3n4-c3ccccc3)cccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2c(-c3ccc4c(c3)c3ccccc3n4-c3ccccc3)cccc2-n2c3ccccc3c3ccccc32)nc(-n2c3ccccc3c3ccccc32)n1] 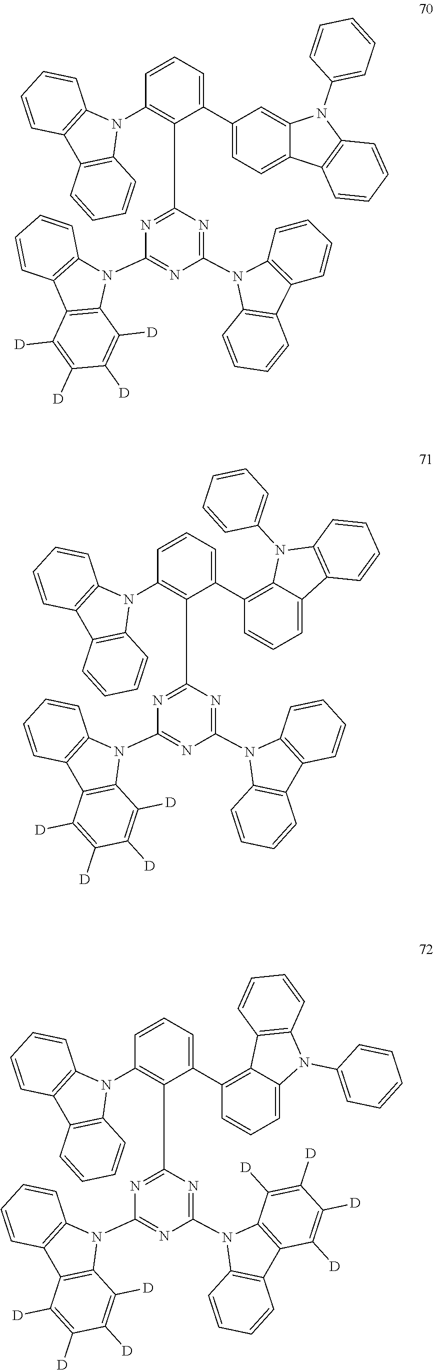 [SMILES: [2H]c1c([2H])c([2H])c2c(c1[2H])c1ccccc1n2-c1nc(-c2c(-c3ccc4c5ccccc5n(-c5ccccc5)c4c3)cccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2c(-c3cccc4c5ccccc5n(-c5ccccc5)c34)cccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2c(-c3cccc4c3c3ccccc3n4-c3ccccc3)cccc2-n2c3ccccc3c3ccccc32)nc(-n2c3ccccc3c3c([2H])c([2H])c([2H])c([2H])c32)n1] 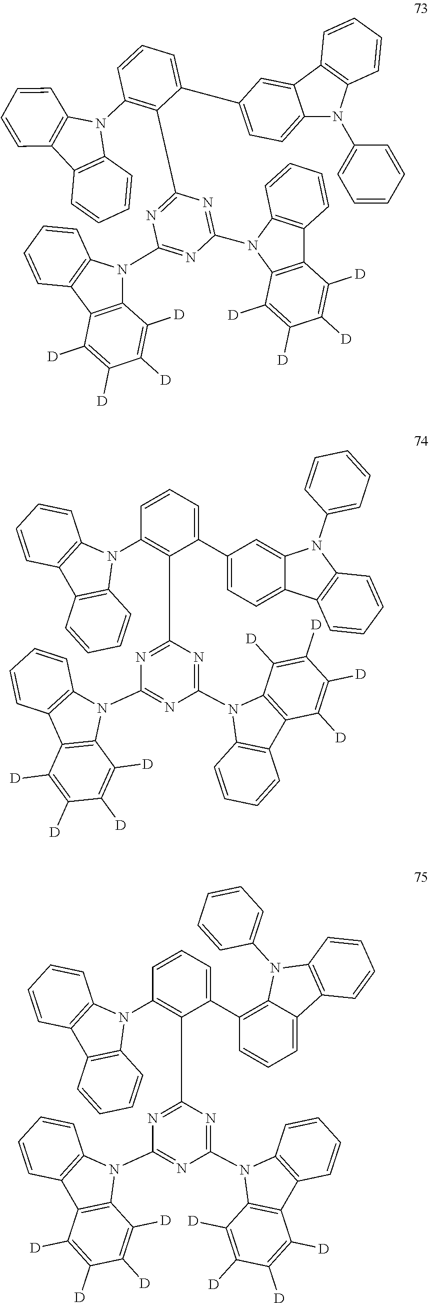 [SMILES: [2H]c1c([2H])c([2H])c2c(c1[2H])c1ccccc1n2-c1nc(-c2c(-c3ccc4c(c3)c3ccccc3n4-c3ccccc3)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2c(-c3ccc4c5ccccc5n(-c5ccccc5)c4c3)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2c(-c3cccc4c5ccccc5n(-c5ccccc5)c34)cccc2-n2c3ccccc3c3ccccc32)nc(-n2c3ccccc3c3c([2H])c([2H])c([2H])c([2H])c32)n1] 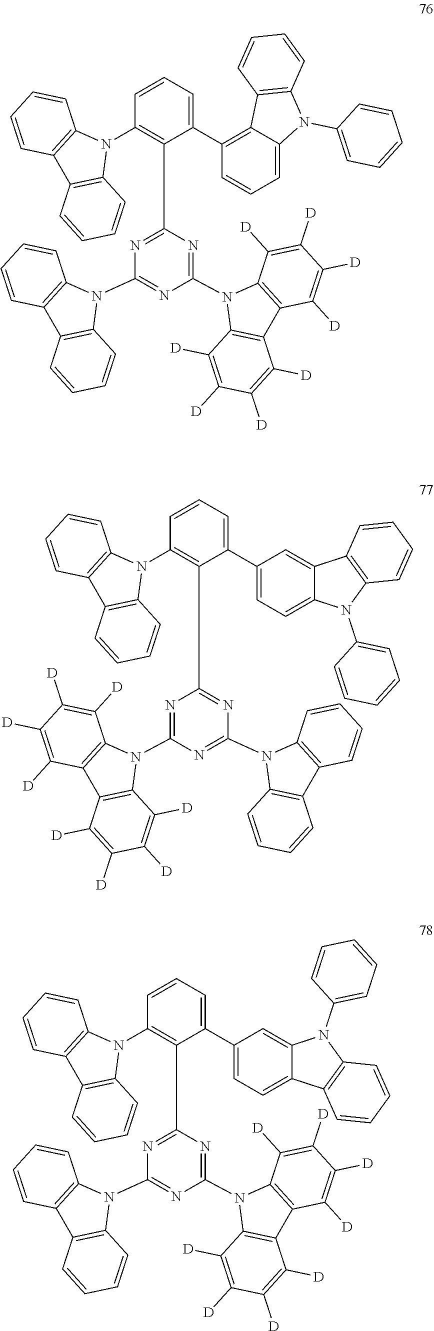 [SMILES: [2H]c1c([2H])c([2H])c2c(c1[2H])c1c([2H])c([2H])c([2H])c([2H])c1n2-c1nc(-c2c(-c3cccc4c3c3ccccc3n4-c3ccccc3)cccc2-n2c3ccccc3c3ccccc32)nc(-n2c3ccccc3c3ccccc32)n1 ; [2H]c1c([2H])c([2H])c2c(c1[2H])c1c([2H])c([2H])c([2H])c([2H])c1n2-c1nc(-c2c(-c3ccc4c(c3)c3ccccc3n4-c3ccccc3)cccc2-n2c3ccccc3c3ccccc32)nc(-n2c3ccccc3c3ccccc32)n1 ; [2H]c1c([2H])c([2H])c2c(c1[2H])c1c([2H])c([2H])c([2H])c([2H])c1n2-c1nc(-c2c(-c3ccc4c5ccccc5n(-c5ccccc5)c4c3)cccc2-n2c3ccccc3c3ccccc32)nc(-n2c3ccccc3c3ccccc32)n1] 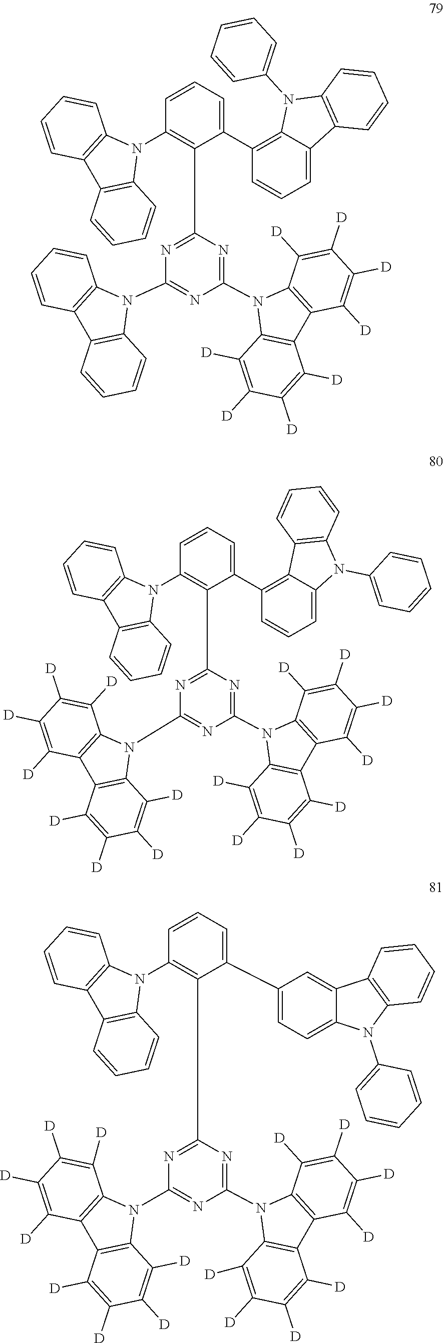 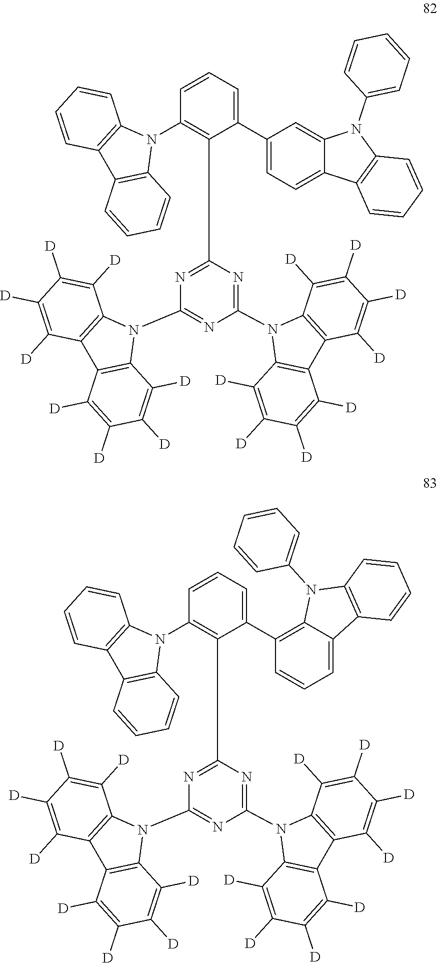 [SMILES: [2H]c1c([2H])c([2H])c2c(c1[2H])c1c([2H])c([2H])c([2H])c([2H])c1n2-c1nc(-c2c(-c3ccc4c5ccccc5n(-c5ccccc5)c4c3)cccc2-n2c3ccccc3c3ccccc32)nc(-n2c3c([2H])c([2H])c([2H])c([2H])c3c3c([2H])c([2H])c([2H])c([2H])c32)n1 ; [2H]c1c([2H])c([2H])c2c(c1[2H])c1c([2H])c([2H])c([2H])c([2H])c1n2-c1nc(-c2c(-c3cccc4c5ccccc5n(-c5ccccc5)c34)cccc2-n2c3ccccc3c3ccccc32)nc(-n2c3c([2H])c([2H])c([2H])c([2H])c3c3c([2H])c([2H])c([2H])c([2H])c32)n1] 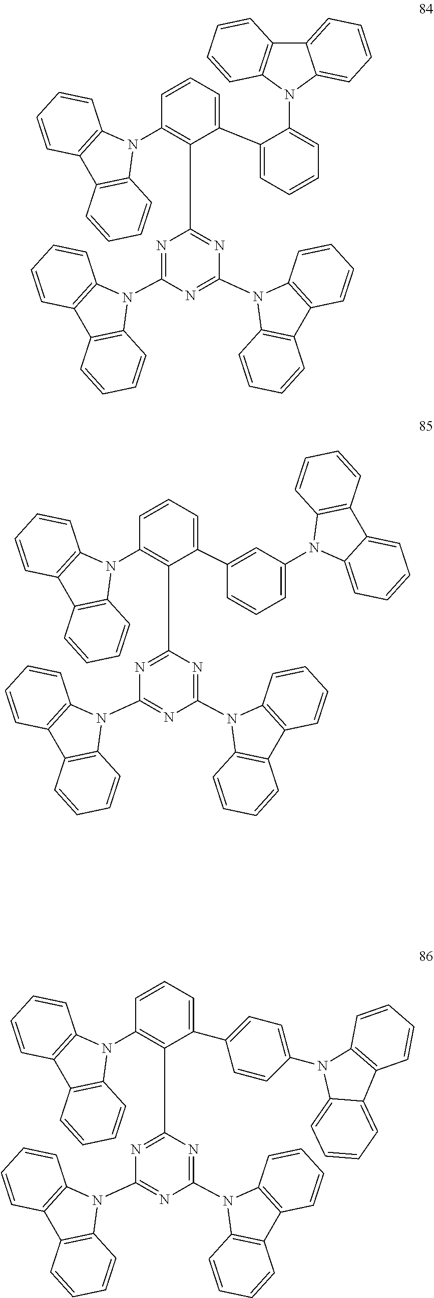 [SMILES: c1ccc(-n2c3ccccc3c3ccccc32)c(-c2cccc(-n3c4ccccc4c4ccccc43)c2-c2nc(-n3c4ccccc4c4ccccc43)nc(-n3c4ccccc4c4ccccc43)n2)c1 ; c1cc(-c2cccc(-n3c4ccccc4c4ccccc43)c2-c2nc(-n3c4ccccc4c4ccccc43)nc(-n3c4ccccc4c4ccccc43)n2)cc(-n2c3ccccc3c3ccccc32)c1 ; c1cc(-c2ccc(-n3c4ccccc4c4ccccc43)cc2)c(-c2nc(-n3c4ccccc4c4ccccc43)nc(-n3c4ccccc4c4ccccc43)n2)c(-n2c3ccccc3c3ccccc32)c1] 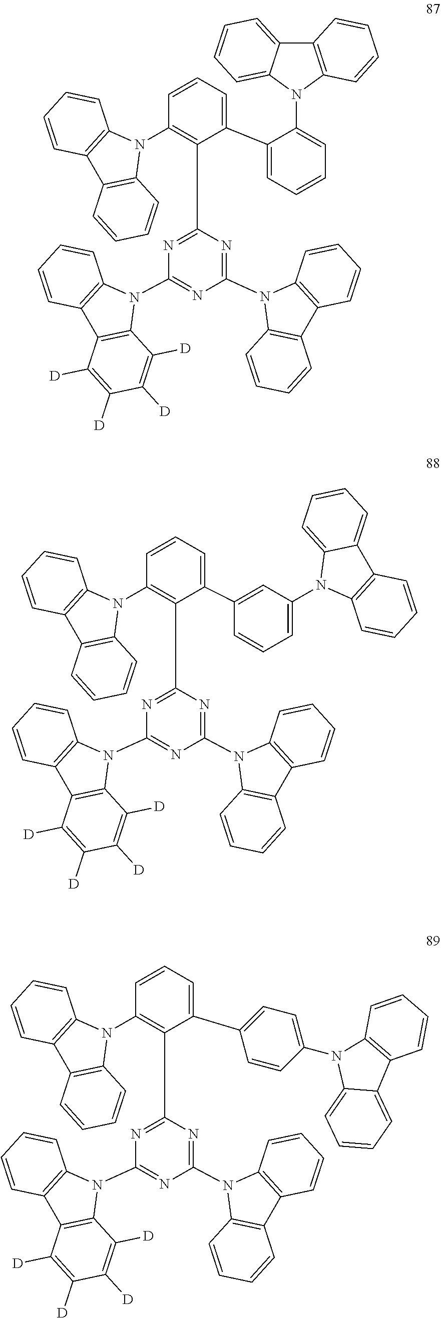 [SMILES: [2H]c1c([2H])c([2H])c2c(c1[2H])c1ccccc1n2-c1nc(-c2c(-c3ccccc3-n3c4ccccc4c4ccccc43)cccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2c(-c3cccc(-n4c5ccccc5c5ccccc54)c3)cccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2c(-c3ccc(-n4c5ccccc5c5ccccc54)cc3)cccc2-n2c3ccccc3c3ccccc32)nc(-n2c3ccccc3c3ccccc32)n1] 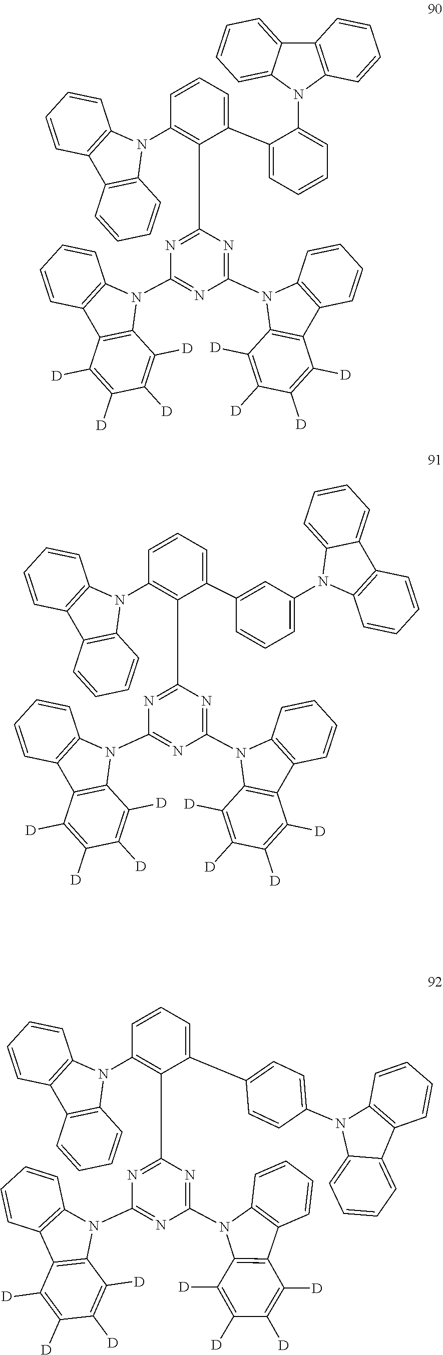 [SMILES: [2H]c1c([2H])c([2H])c2c(c1[2H])c1ccccc1n2-c1nc(-c2c(-c3ccccc3-n3c4ccccc4c4ccccc43)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2c(-c3cccc(-n4c5ccccc5c5ccccc54)c3)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2c(-c3ccc(-n4c5ccccc5c5ccccc54)cc3)cccc2-n2c3ccccc3c3ccccc32)nc(-n2c3ccccc3c3c([2H])c([2H])c([2H])c([2H])c32)n1] 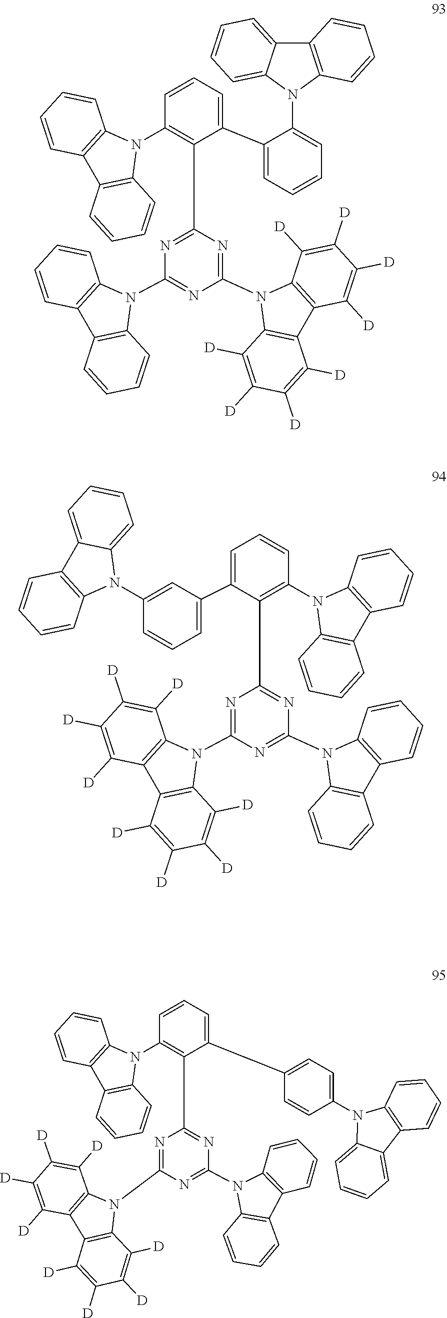 [SMILES: [2H]c1c([2H])c([2H])c2c(c1[2H])c1c([2H])c([2H])c([2H])c([2H])c1n2-c1nc(-c2c(-c3ccccc3-n3c4ccccc4c4ccccc43)cccc2-n2c3ccccc3c3ccccc32)nc(-n2c3ccccc3c3ccccc32)n1 ; [2H]c1c([2H])c([2H])c2c(c1[2H])c1c([2H])c([2H])c([2H])c([2H])c1n2-c1nc(-c2c(-c3cccc(-n4c5ccccc5c5ccccc54)c3)cccc2-n2c3ccccc3c3ccccc32)nc(-n2c3ccccc3c3ccccc32)n1 ; [2H]c1c([2H])c([2H])c2c(c1[2H])c1c([2H])c([2H])c([2H])c([2H])c1n2-c1nc(-c2c(-c3ccc(-n4c5ccccc5c5ccccc54)cc3)cccc2-n2c3ccccc3c3ccccc32)nc(-n2c3ccccc3c3ccccc32)n1] 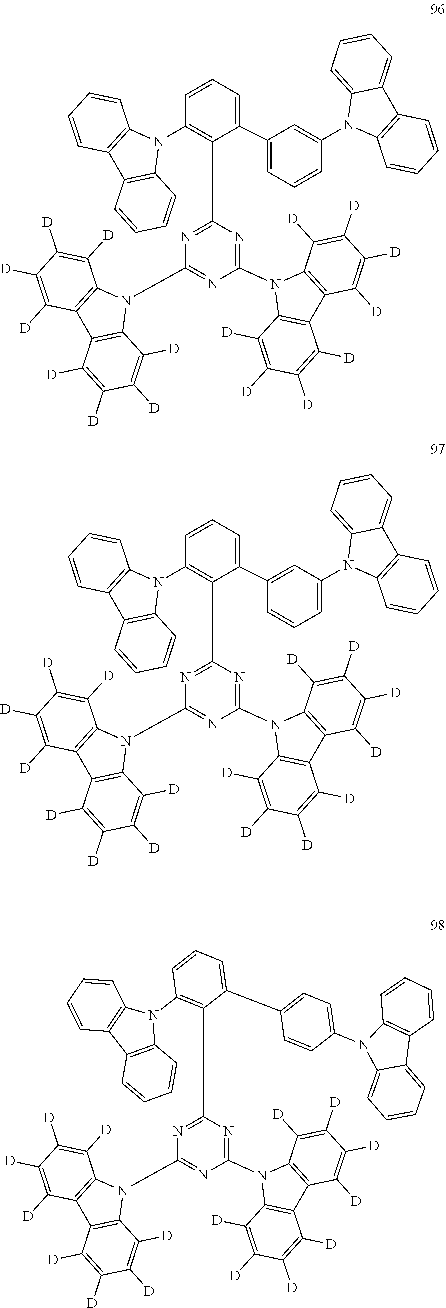 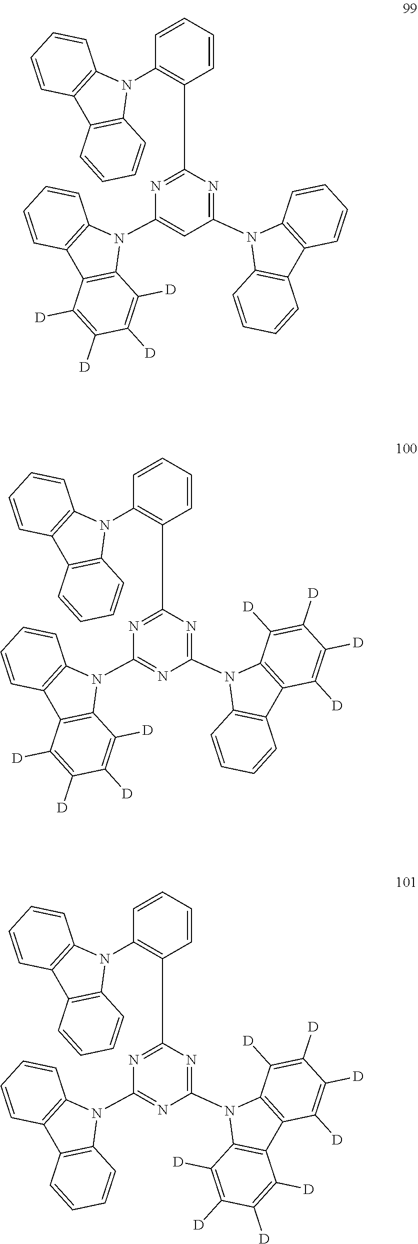 [SMILES: [2H]c1c([2H])c([2H])c2c(c1[2H])c1ccccc1n2-c1cc(-n2c3ccccc3c3ccccc32)nc(-c2ccccc2-n2c3ccccc3c3ccccc32)n1 ; [2H]c1c([2H])c([2H])c2c(c1[2H])c1ccccc1n2-c1nc(-c2ccccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1c([2H])c([2H])c([2H])c([2H])c1n2-c1nc(-c2ccccc2-n2c3ccccc3c3ccccc32)nc(-n2c3ccccc3c3ccccc32)n1] 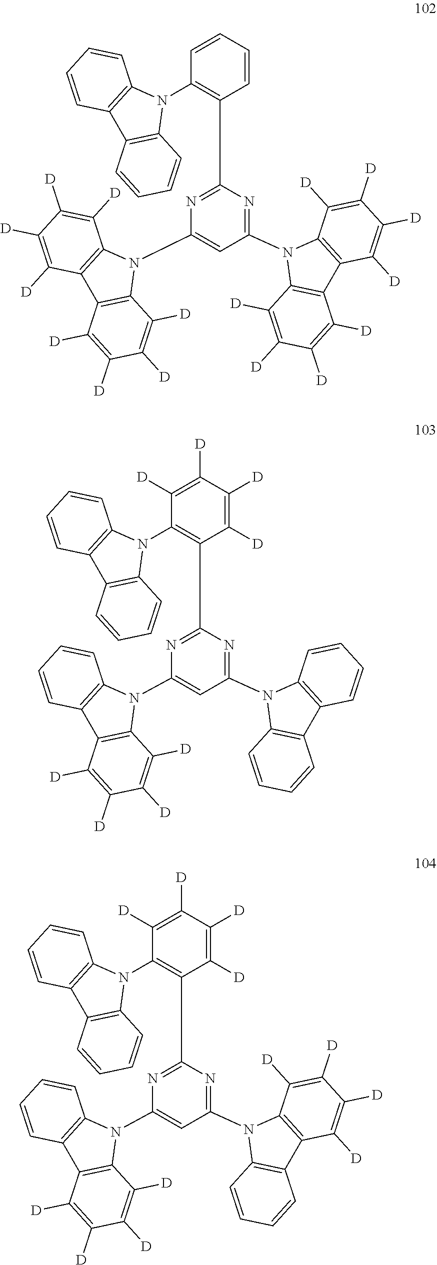 [SMILES: [2H]c1c([2H])c([2H])c2c(c1[2H])c1c([2H])c([2H])c([2H])c([2H])c1n2-c1cc(-n2c3c([2H])c([2H])c([2H])c([2H])c3c3c([2H])c([2H])c([2H])c([2H])c32)nc(-c2ccccc2-n2c3ccccc3c3ccccc32)n1 ; [2H]c1c([2H])c([2H])c(-n2c3ccccc3c3ccccc32)c(-c2nc(-n3c4ccccc4c4ccccc43)cc(-n3c4ccccc4c4c([2H])c([2H])c([2H])c([2H])c43)n2)c1[2H] ; [2H]c1c([2H])c([2H])c(-n2c3ccccc3c3ccccc32)c(-c2nc(-n3c4ccccc4c4c([2H])c([2H])c([2H])c([2H])c43)cc(-n3c4ccccc4c4c([2H])c([2H])c([2H])c([2H])c43)n2)c1[2H]] 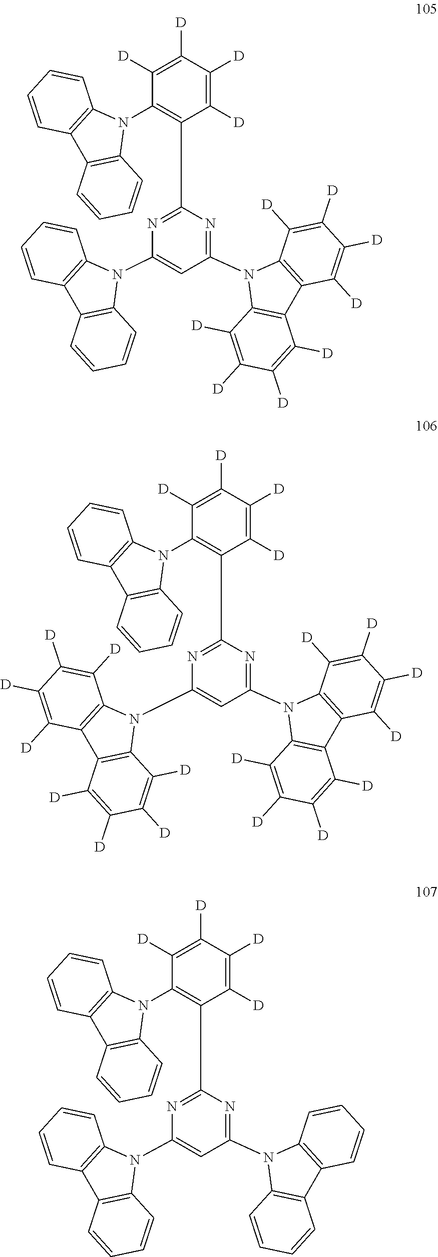 [SMILES: [2H]c1c([2H])c([2H])c(-n2c3ccccc3c3ccccc32)c(-c2nc(-n3c4ccccc4c4ccccc43)cc(-n3c4c([2H])c([2H])c([2H])c([2H])c4c4c([2H])c([2H])c([2H])c([2H])c43)n2)c1[2H] ; [2H]c1c([2H])c([2H])c(-n2c3ccccc3c3ccccc32)c(-c2nc(-n3c4c([2H])c([2H])c([2H])c([2H])c4c4c([2H])c([2H])c([2H])c([2H])c43)cc(-n3c4c([2H])c([2H])c([2H])c([2H])c4c4c([2H])c([2H])c([2H])c([2H])c43)n2)c1[2H] ; [2H]c1c([2H])c([2H])c(-n2c3ccccc3c3ccccc32)c(-c2nc(-n3c4ccccc4c4ccccc43)cc(-n3c4ccccc4c4ccccc43)n2)c1[2H]] 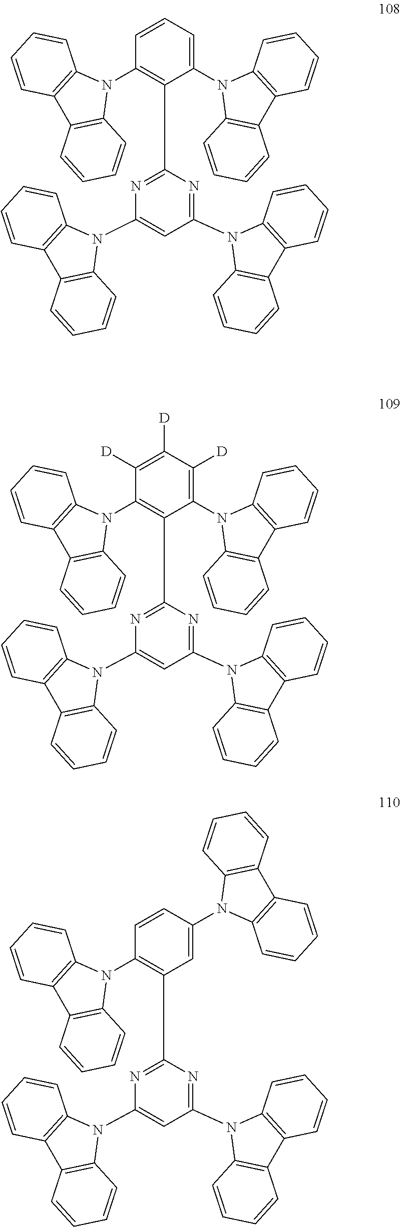 [SMILES: c1cc(-n2c3ccccc3c3ccccc32)c(-c2nc(-n3c4ccccc4c4ccccc43)cc(-n3c4ccccc4c4ccccc43)n2)c(-n2c3ccccc3c3ccccc32)c1 ; [2H]c1c([2H])c(-n2c3ccccc3c3ccccc32)c(-c2nc(-n3c4ccccc4c4ccccc43)cc(-n3c4ccccc4c4ccccc43)n2)c(-n2c3ccccc3c3ccccc32)c1[2H] ; c1ccc2c(c1)c1ccccc1n2-c1ccc(-n2c3ccccc3c3ccccc32)c(-c2nc(-n3c4ccccc4c4ccccc43)cc(-n3c4ccccc4c4ccccc43)n2)c1] 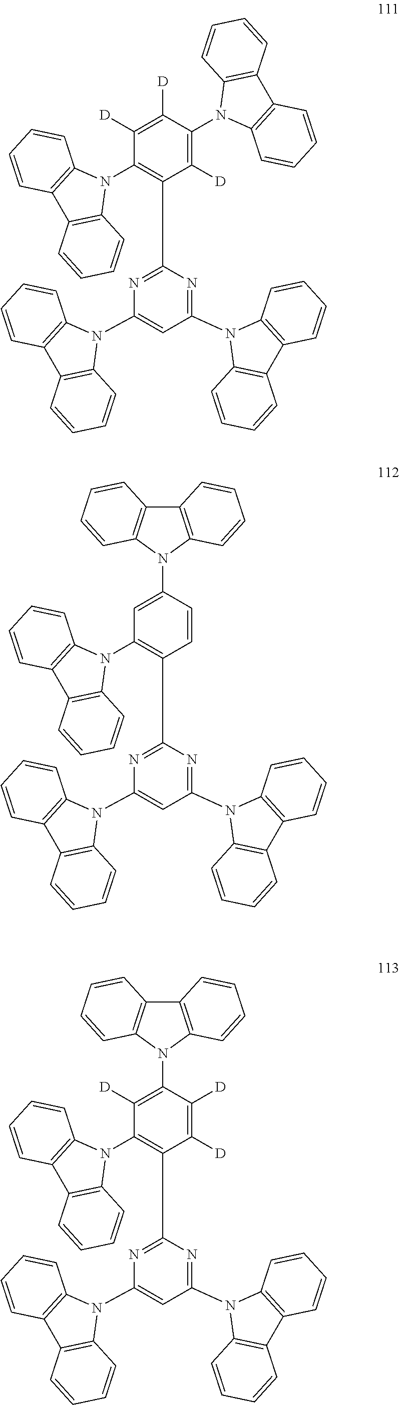 [SMILES: [2H]c1c([2H])c(-n2c3ccccc3c3ccccc32)c(-c2nc(-n3c4ccccc4c4ccccc43)cc(-n3c4ccccc4c4ccccc43)n2)c([2H])c1-n1c2ccccc2c2ccccc21 ; c1ccc2c(c1)c1ccccc1n2-c1ccc(-c2nc(-n3c4ccccc4c4ccccc43)cc(-n3c4ccccc4c4ccccc43)n2)c(-n2c3ccccc3c3ccccc32)c1 ; [2H]c1c([2H])c(-n2c3ccccc3c3ccccc32)c([2H])c(-n2c3ccccc3c3ccccc32)c1-c1nc(-n2c3ccccc3c3ccccc32)cc(-n2c3ccccc3c3ccccc32)n1] 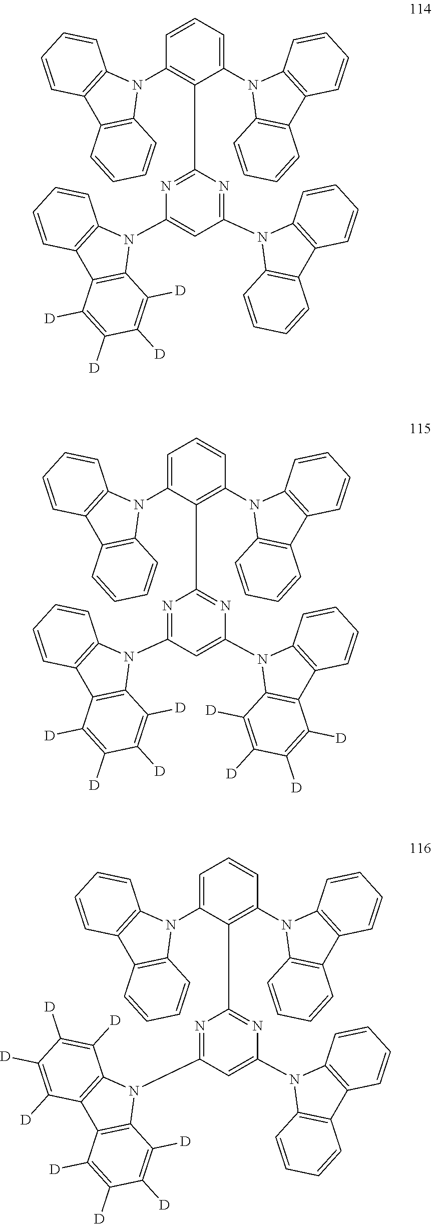 [SMILES: [2H]c1c([2H])c([2H])c2c(c1[2H])c1ccccc1n2-c1cc(-n2c3ccccc3c3ccccc32)nc(-c2c(-n3c4ccccc4c4ccccc43)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-n3c4ccccc4c4ccccc43)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3ccccc3c3ccccc32)nc(-c2c(-n3c4ccccc4c4ccccc43)cccc2-n2c3ccccc3c3ccccc32)n1] 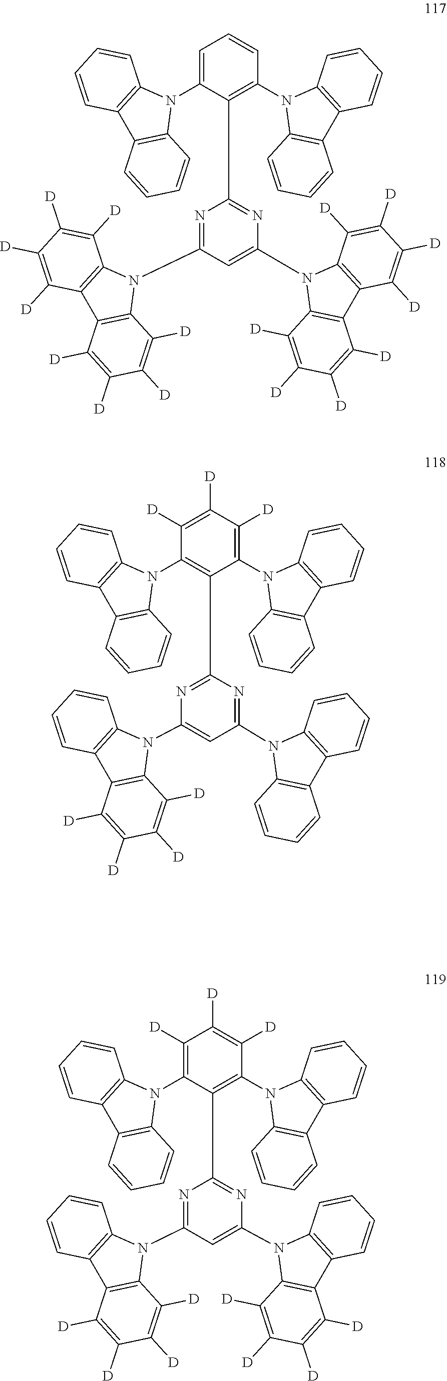 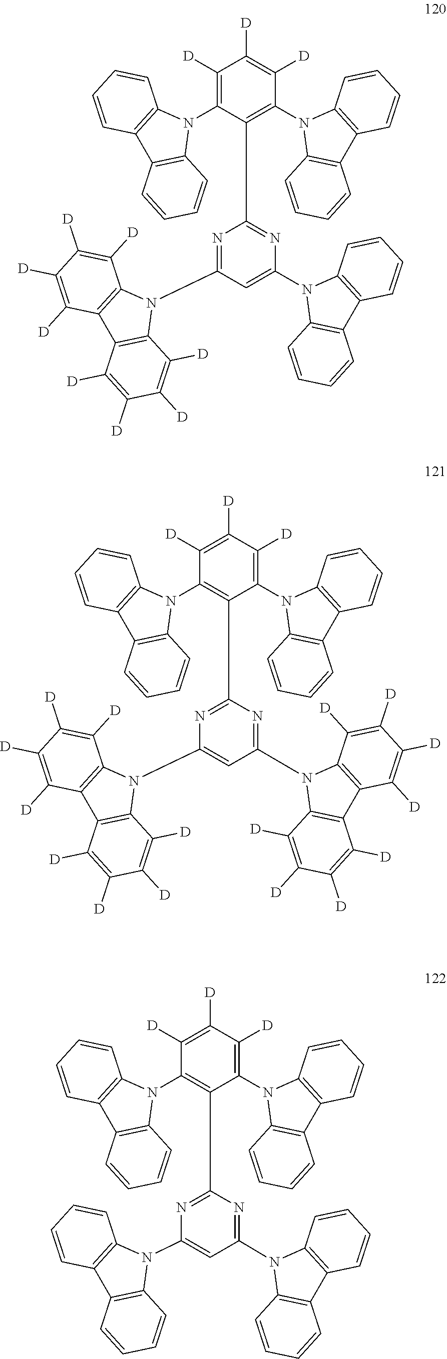 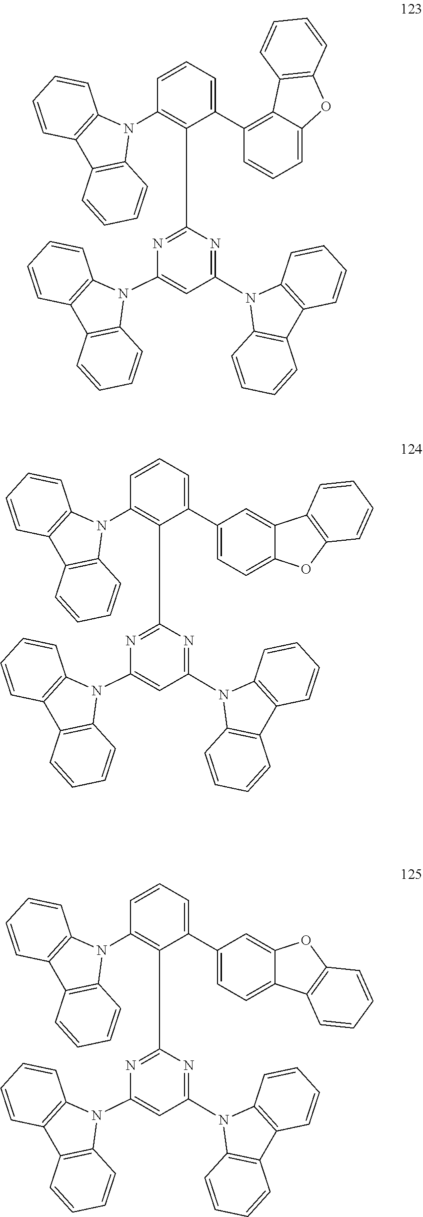 [SMILES: c1cc(-c2cccc3oc4ccccc4c23)c(-c2nc(-n3c4ccccc4c4ccccc43)cc(-n3c4ccccc4c4ccccc43)n2)c(-n2c3ccccc3c3ccccc32)c1 ; c1cc(-c2ccc3oc4ccccc4c3c2)c(-c2nc(-n3c4ccccc4c4ccccc43)cc(-n3c4ccccc4c4ccccc43)n2)c(-n2c3ccccc3c3ccccc32)c1 ; c1cc(-c2ccc3c(c2)oc2ccccc23)c(-c2nc(-n3c4ccccc4c4ccccc43)cc(-n3c4ccccc4c4ccccc43)n2)c(-n2c3ccccc3c3ccccc32)c1] 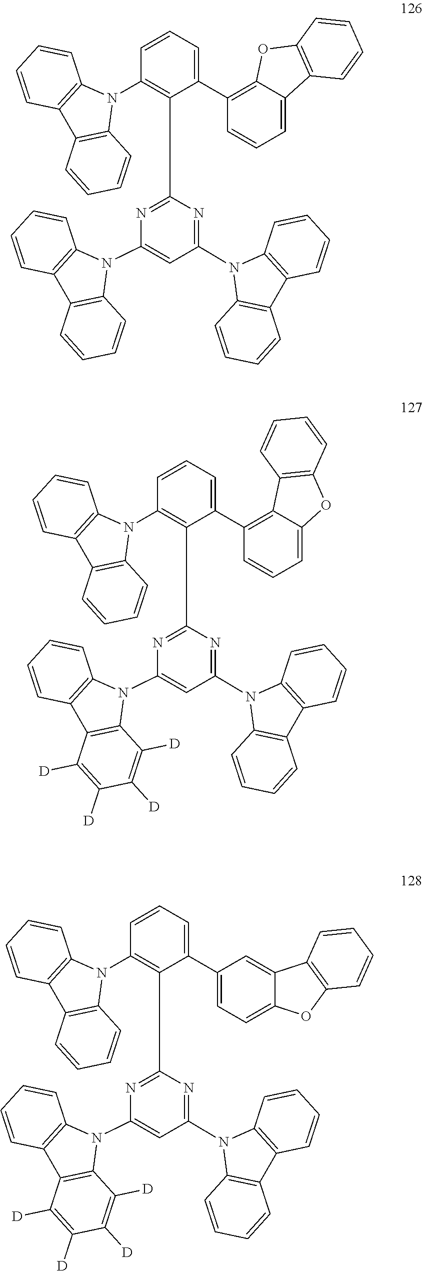 [SMILES: c1cc(-c2cccc3c2oc2ccccc23)c(-c2nc(-n3c4ccccc4c4ccccc43)cc(-n3c4ccccc4c4ccccc43)n2)c(-n2c3ccccc3c3ccccc32)c1 ; [2H]c1c([2H])c([2H])c2c(c1[2H])c1ccccc1n2-c1cc(-n2c3ccccc3c3ccccc32)nc(-c2c(-c3cccc4oc5ccccc5c34)cccc2-n2c3ccccc3c3ccccc32)n1 ; [2H]c1c([2H])c([2H])c2c(c1[2H])c1ccccc1n2-c1cc(-n2c3ccccc3c3ccccc32)nc(-c2c(-c3ccc4oc5ccccc5c4c3)cccc2-n2c3ccccc3c3ccccc32)n1] 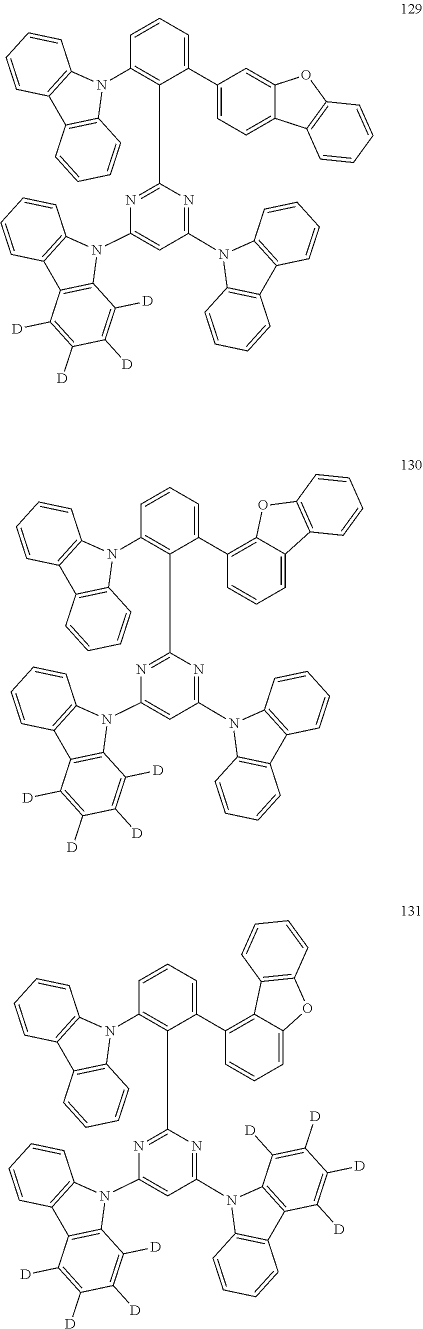 [SMILES: [2H]c1c([2H])c([2H])c2c(c1[2H])c1ccccc1n2-c1cc(-n2c3ccccc3c3ccccc32)nc(-c2c(-c3ccc4c(c3)oc3ccccc34)cccc2-n2c3ccccc3c3ccccc32)n1 ; [2H]c1c([2H])c([2H])c2c(c1[2H])c1ccccc1n2-c1cc(-n2c3ccccc3c3ccccc32)nc(-c2c(-c3cccc4c3oc3ccccc34)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-c3cccc4oc5ccccc5c34)cccc2-n2c3ccccc3c3ccccc32)n1] 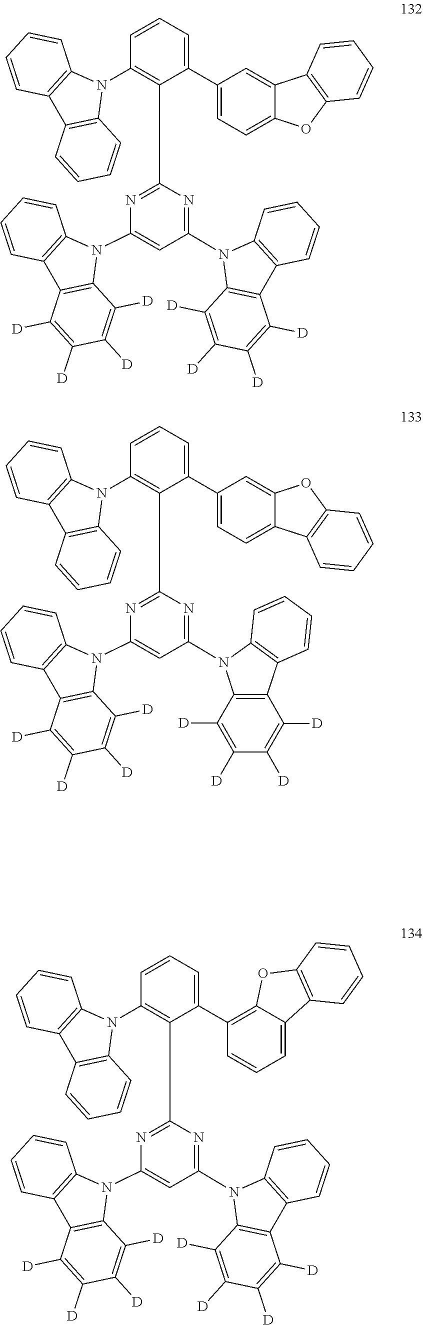 [SMILES: [2H]c1c([2H])c([2H])c2c(c1[2H])c1ccccc1n2-c1cc(-n2c3ccccc3c3c([2H])c([2H])c([2H])c([2H])c32)nc(-c2c(-c3ccc4oc5ccccc5c4c3)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-c3ccc4c(c3)oc3ccccc34)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-c3cccc4c3oc3ccccc34)cccc2-n2c3ccccc3c3ccccc32)n1] 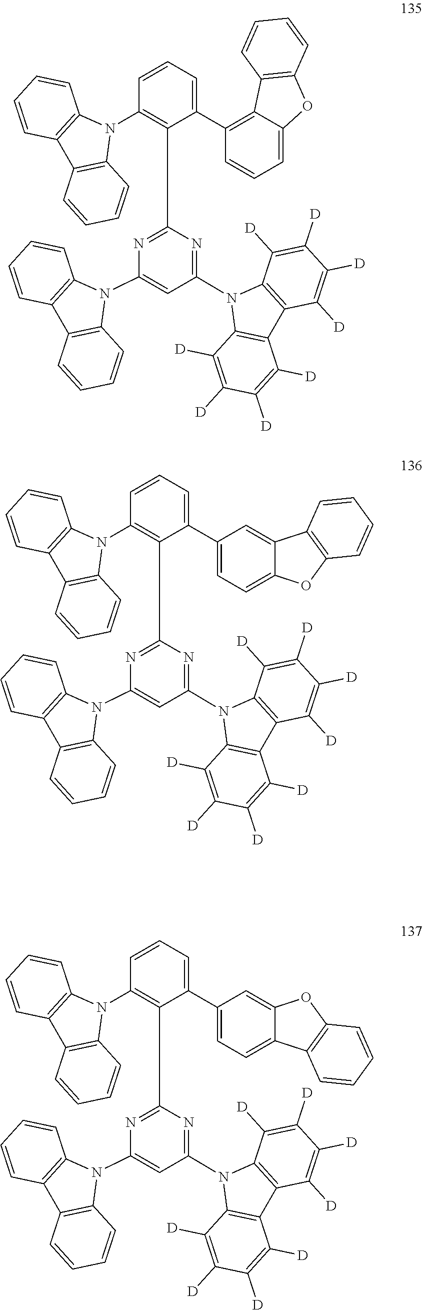 [SMILES: [2H]c1c([2H])c([2H])c2c(c1[2H])c1c([2H])c([2H])c([2H])c([2H])c1n2-c1cc(-n2c3ccccc3c3ccccc32)nc(-c2c(-c3cccc4oc5ccccc5c34)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3ccccc3c3ccccc32)nc(-c2c(-c3ccc4oc5ccccc5c4c3)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3ccccc3c3ccccc32)nc(-c2c(-c3ccc4c(c3)oc3ccccc34)cccc2-n2c3ccccc3c3ccccc32)n1] 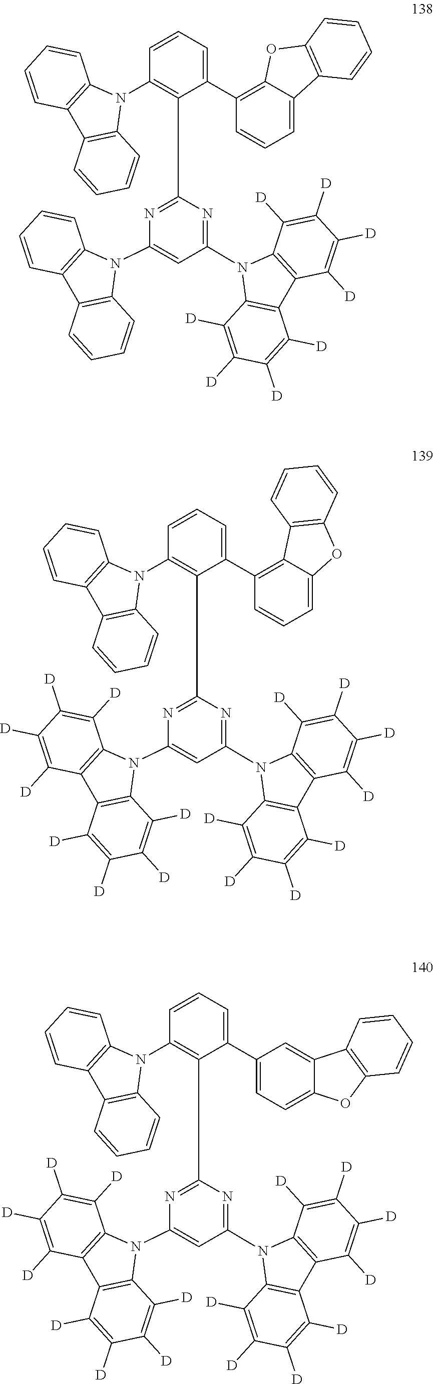 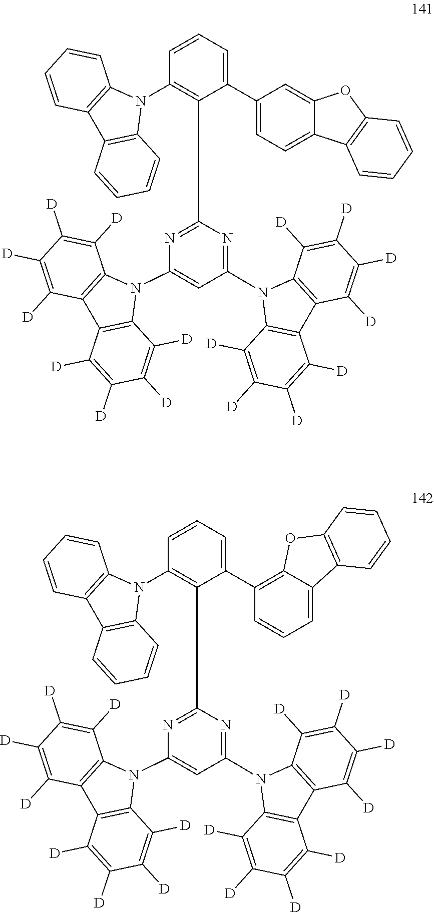 [SMILES: [2H]c1c([2H])c([2H])c2c(c1[2H])c1c([2H])c([2H])c([2H])c([2H])c1n2-c1cc(-n2c3c([2H])c([2H])c([2H])c([2H])c3c3c([2H])c([2H])c([2H])c([2H])c32)nc(-c2c(-c3ccc4c(c3)oc3ccccc34)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3c([2H])c([2H])c([2H])c([2H])c3c3c([2H])c([2H])c([2H])c([2H])c32)nc(-c2c(-c3cccc4c3oc3ccccc34)cccc2-n2c3ccccc3c3ccccc32)n1] 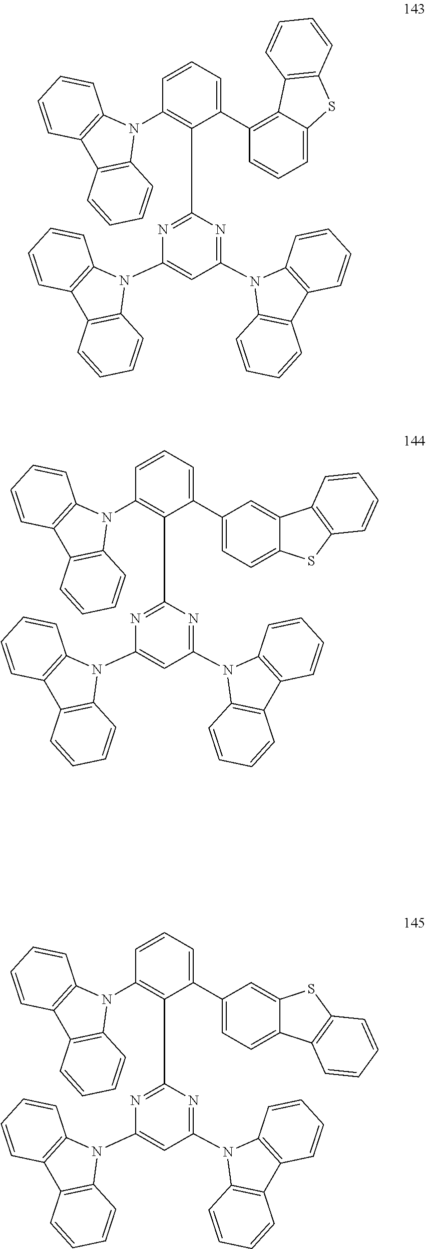 [SMILES: c1cc(-c2cccc3sc4ccccc4c23)c(-c2nc(-n3c4ccccc4c4ccccc43)cc(-n3c4ccccc4c4ccccc43)n2)c(-n2c3ccccc3c3ccccc32)c1 ; c1cc(-c2ccc3sc4ccccc4c3c2)c(-c2nc(-n3c4ccccc4c4ccccc43)cc(-n3c4ccccc4c4ccccc43)n2)c(-n2c3ccccc3c3ccccc32)c1 ; c1cc(-c2ccc3c(c2)sc2ccccc23)c(-c2nc(-n3c4ccccc4c4ccccc43)cc(-n3c4ccccc4c4ccccc43)n2)c(-n2c3ccccc3c3ccccc32)c1] 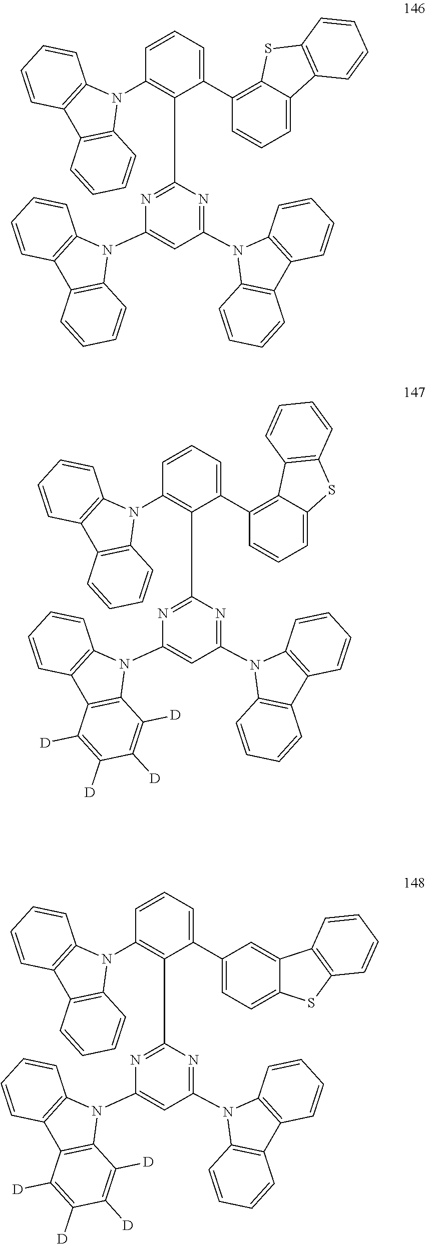 [SMILES: c1cc(-c2cccc3c2sc2ccccc23)c(-c2nc(-n3c4ccccc4c4ccccc43)cc(-n3c4ccccc4c4ccccc43)n2)c(-n2c3ccccc3c3ccccc32)c1 ; [2H]c1c([2H])c([2H])c2c(c1[2H])c1ccccc1n2-c1cc(-n2c3ccccc3c3ccccc32)nc(-c2c(-c3cccc4sc5ccccc5c34)cccc2-n2c3ccccc3c3ccccc32)n1 ; [2H]c1c([2H])c([2H])c2c(c1[2H])c1ccccc1n2-c1cc(-n2c3ccccc3c3ccccc32)nc(-c2c(-c3ccc4sc5ccccc5c4c3)cccc2-n2c3ccccc3c3ccccc32)n1] 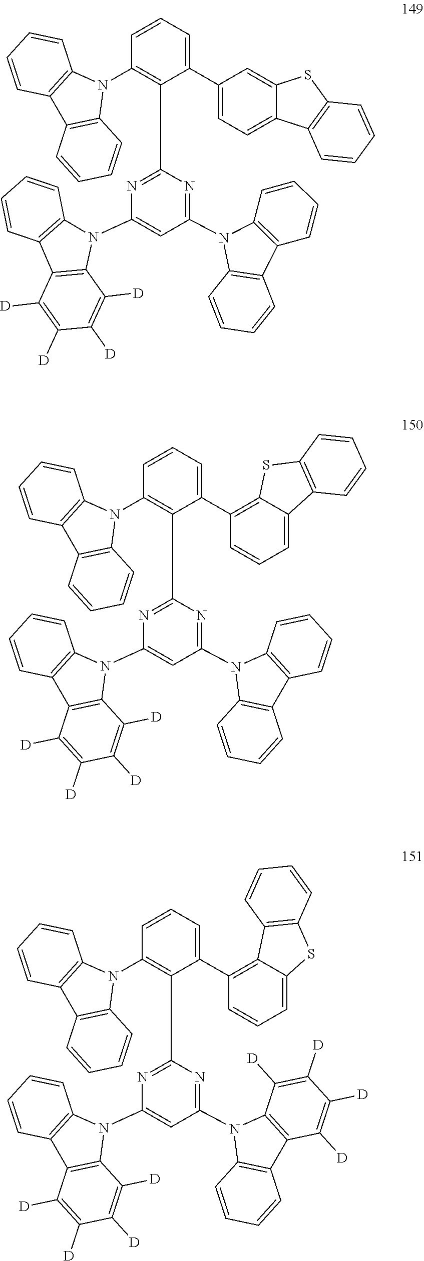 [SMILES: [2H]c1c([2H])c([2H])c2c(c1[2H])c1ccccc1n2-c1cc(-n2c3ccccc3c3ccccc32)nc(-c2c(-c3ccc4c(c3)sc3ccccc34)cccc2-n2c3ccccc3c3ccccc32)n1 ; [2H]c1c([2H])c([2H])c2c(c1[2H])c1ccccc1n2-c1cc(-n2c3ccccc3c3ccccc32)nc(-c2c(-c3cccc4c3sc3ccccc34)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-c3cccc4sc5ccccc5c34)cccc2-n2c3ccccc3c3ccccc32)n1] 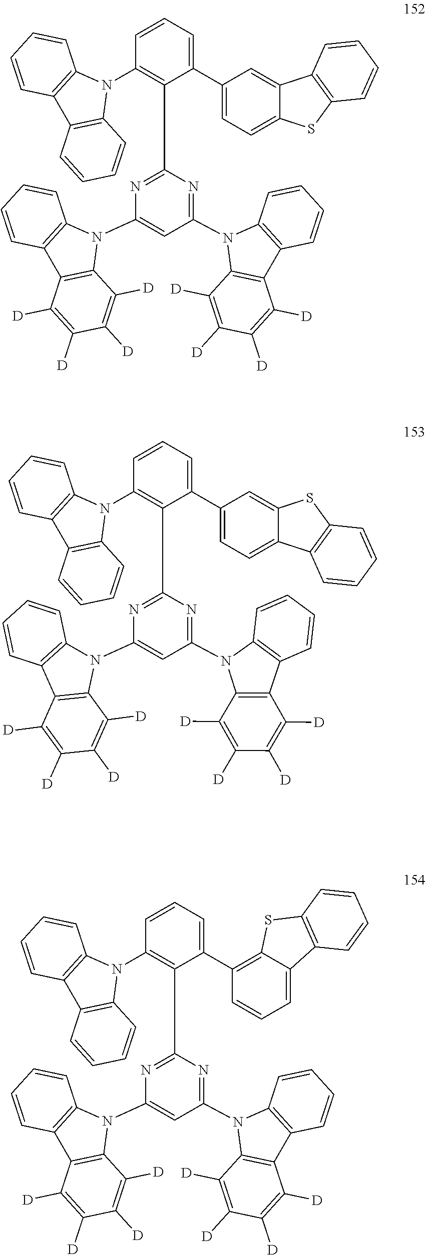 [SMILES: [2H]c1c([2H])c([2H])c2c(c1[2H])c1ccccc1n2-c1cc(-n2c3ccccc3c3c([2H])c([2H])c([2H])c([2H])c32)nc(-c2c(-c3ccc4sc5ccccc5c4c3)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-c3ccc4c(c3)sc3ccccc34)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-c3cccc4c3sc3ccccc34)cccc2-n2c3ccccc3c3ccccc32)n1] 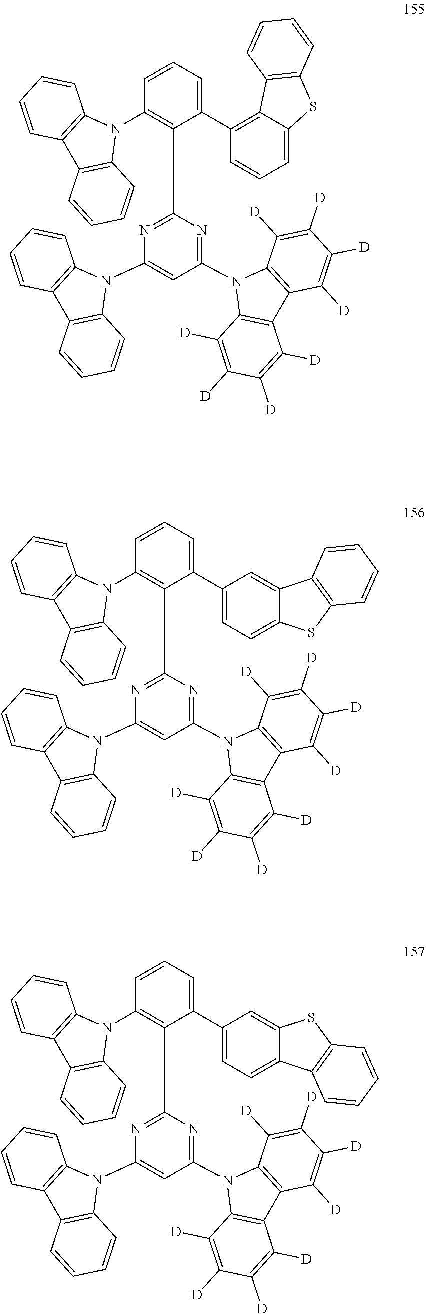 [SMILES: [2H]c1c([2H])c([2H])c2c(c1[2H])c1c([2H])c([2H])c([2H])c([2H])c1n2-c1cc(-n2c3ccccc3c3ccccc32)nc(-c2c(-c3cccc4sc5ccccc5c34)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3ccccc3c3ccccc32)nc(-c2c(-c3ccc4sc5ccccc5c4c3)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3ccccc3c3ccccc32)nc(-c2c(-c3ccc4c(c3)sc3ccccc34)cccc2-n2c3ccccc3c3ccccc32)n1] 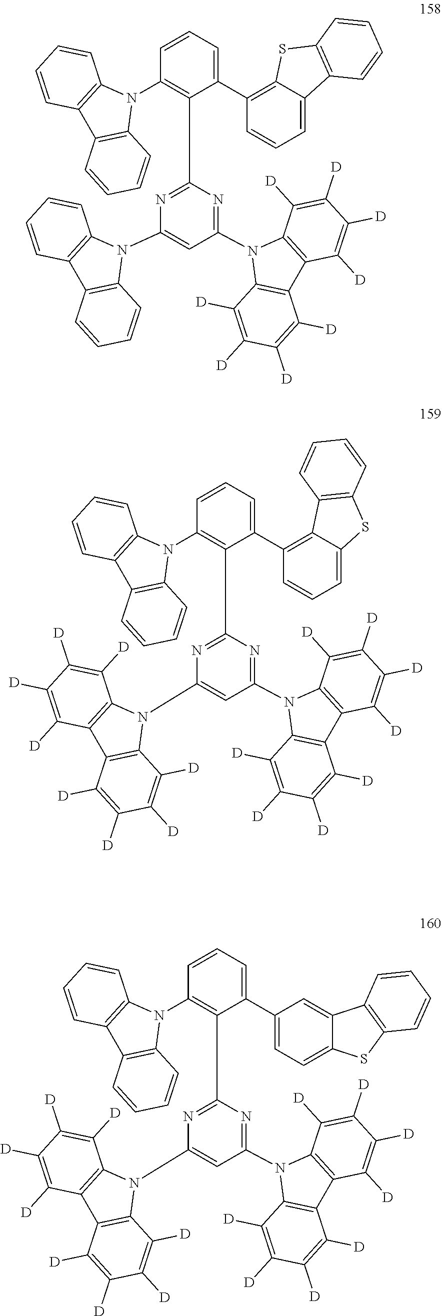 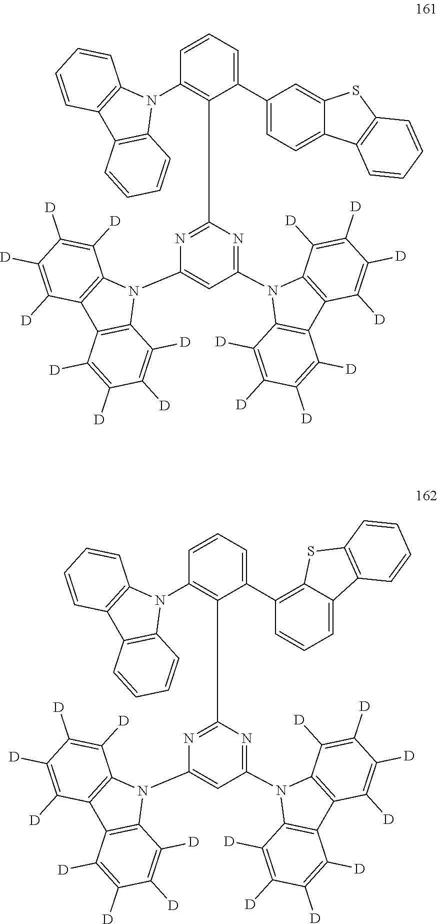 [SMILES: [2H]c1c([2H])c([2H])c2c(c1[2H])c1c([2H])c([2H])c([2H])c([2H])c1n2-c1cc(-n2c3c([2H])c([2H])c([2H])c([2H])c3c3c([2H])c([2H])c([2H])c([2H])c32)nc(-c2c(-c3ccc4c(c3)sc3ccccc34)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3c([2H])c([2H])c([2H])c([2H])c3c3c([2H])c([2H])c([2H])c([2H])c32)nc(-c2c(-c3cccc4c3sc3ccccc34)cccc2-n2c3ccccc3c3ccccc32)n1] 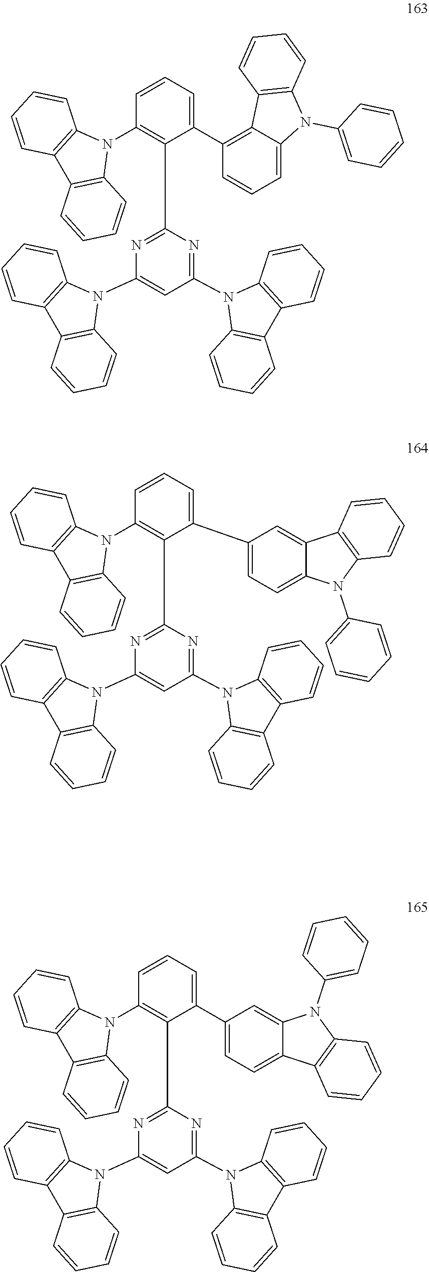 [SMILES: c1ccc(-n2c3ccccc3c3c(-c4cccc(-n5c6ccccc6c6ccccc65)c4-c4nc(-n5c6ccccc6c6ccccc65)cc(-n5c6ccccc6c6ccccc65)n4)cccc32)cc1 ; c1ccc(-n2c3ccccc3c3cc(-c4cccc(-n5c6ccccc6c6ccccc65)c4-c4nc(-n5c6ccccc6c6ccccc65)cc(-n5c6ccccc6c6ccccc65)n4)ccc32)cc1 ; c1ccc(-n2c3ccccc3c3ccc(-c4cccc(-n5c6ccccc6c6ccccc65)c4-c4nc(-n5c6ccccc6c6ccccc65)cc(-n5c6ccccc6c6ccccc65)n4)cc32)cc1] 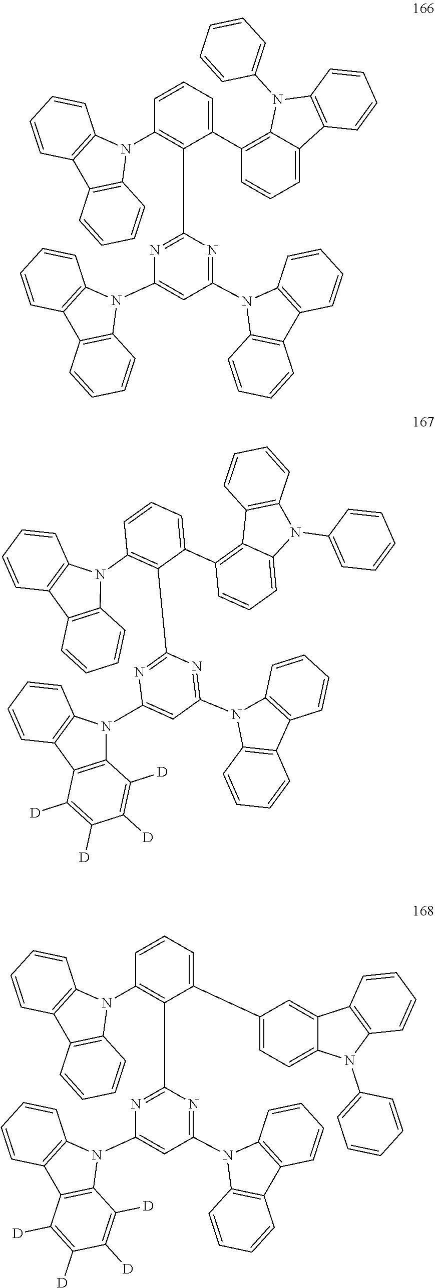 [SMILES: c1ccc(-n2c3ccccc3c3cccc(-c4cccc(-n5c6ccccc6c6ccccc65)c4-c4nc(-n5c6ccccc6c6ccccc65)cc(-n5c6ccccc6c6ccccc65)n4)c32)cc1 ; [2H]c1c([2H])c([2H])c2c(c1[2H])c1ccccc1n2-c1cc(-n2c3ccccc3c3ccccc32)nc(-c2c(-c3cccc4c3c3ccccc3n4-c3ccccc3)cccc2-n2c3ccccc3c3ccccc32)n1 ; [2H]c1c([2H])c([2H])c2c(c1[2H])c1ccccc1n2-c1cc(-n2c3ccccc3c3ccccc32)nc(-c2c(-c3ccc4c(c3)c3ccccc3n4-c3ccccc3)cccc2-n2c3ccccc3c3ccccc32)n1] 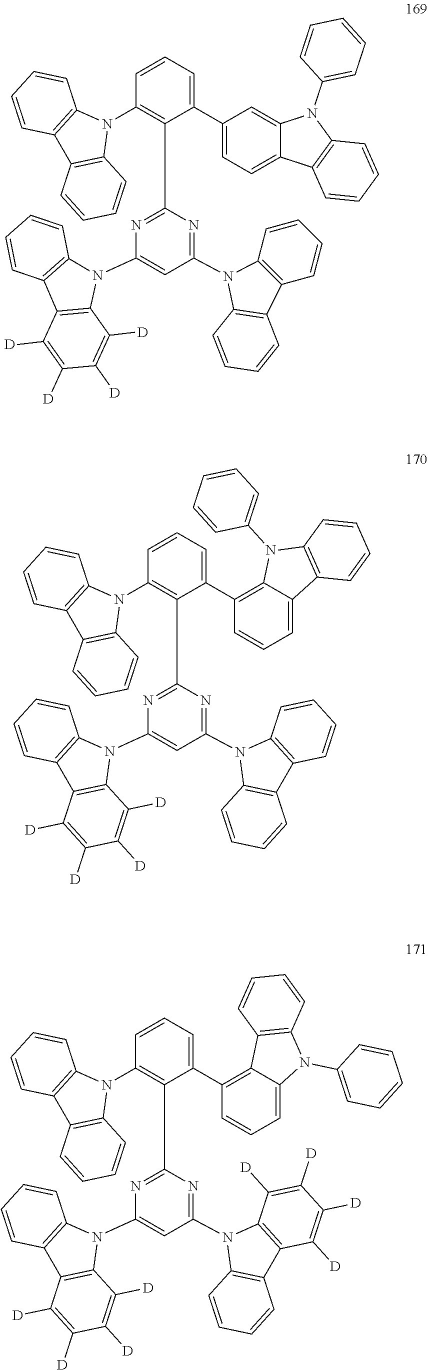 [SMILES: [2H]c1c([2H])c([2H])c2c(c1[2H])c1ccccc1n2-c1cc(-n2c3ccccc3c3ccccc32)nc(-c2c(-c3ccc4c5ccccc5n(-c5ccccc5)c4c3)cccc2-n2c3ccccc3c3ccccc32)n1 ; [2H]c1c([2H])c([2H])c2c(c1[2H])c1ccccc1n2-c1cc(-n2c3ccccc3c3ccccc32)nc(-c2c(-c3cccc4c5ccccc5n(-c5ccccc5)c34)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-c3cccc4c3c3ccccc3n4-c3ccccc3)cccc2-n2c3ccccc3c3ccccc32)n1] 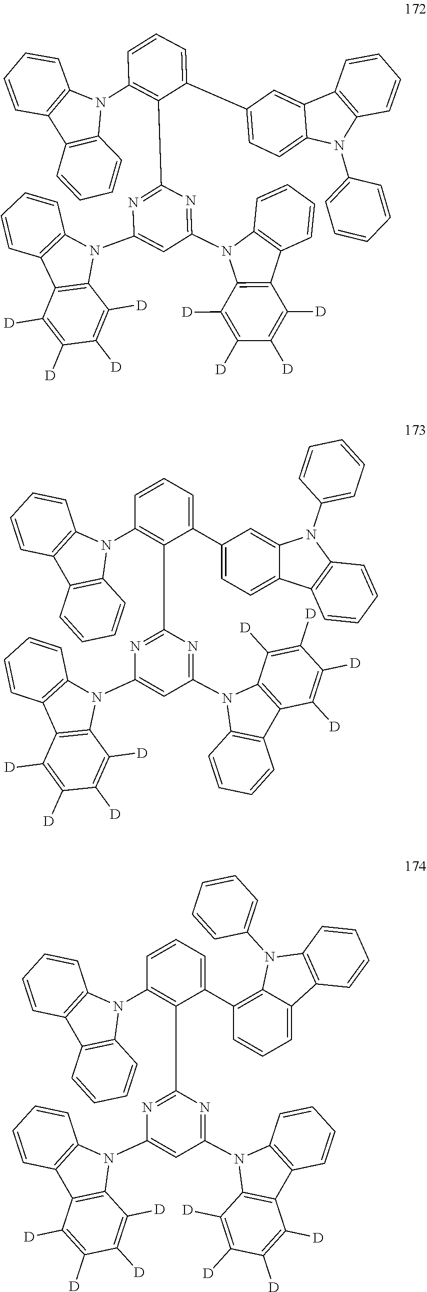 [SMILES: [2H]c1c([2H])c([2H])c2c(c1[2H])c1ccccc1n2-c1cc(-n2c3ccccc3c3c([2H])c([2H])c([2H])c([2H])c32)nc(-c2c(-c3ccc4c(c3)c3ccccc3n4-c3ccccc3)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-c3ccc4c5ccccc5n(-c5ccccc5)c4c3)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-c3cccc4c5ccccc5n(-c5ccccc5)c34)cccc2-n2c3ccccc3c3ccccc32)n1] 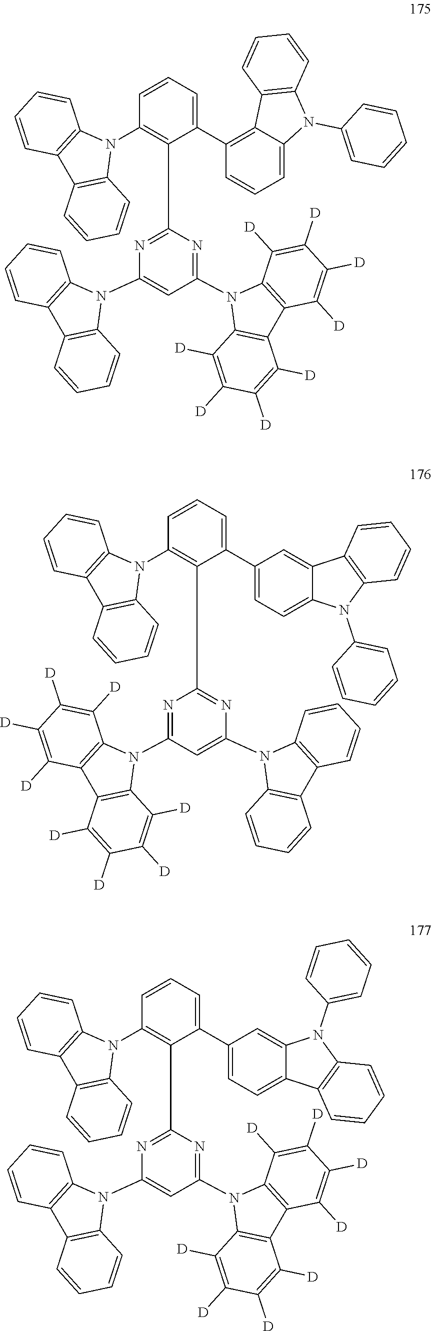 [SMILES: [2H]c1c([2H])c([2H])c2c(c1[2H])c1c([2H])c([2H])c([2H])c([2H])c1n2-c1cc(-n2c3ccccc3c3ccccc32)nc(-c2c(-c3cccc4c3c3ccccc3n4-c3ccccc3)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3ccccc3c3ccccc32)nc(-c2c(-c3ccc4c(c3)c3ccccc3n4-c3ccccc3)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3ccccc3c3ccccc32)nc(-c2c(-c3ccc4c5ccccc5n(-c5ccccc5)c4c3)cccc2-n2c3ccccc3c3ccccc32)n1] 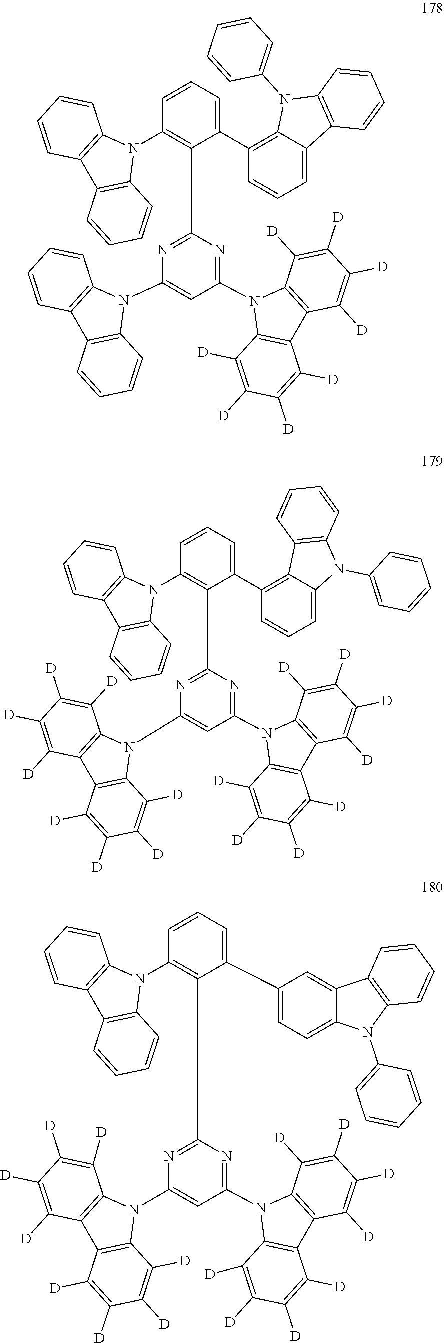 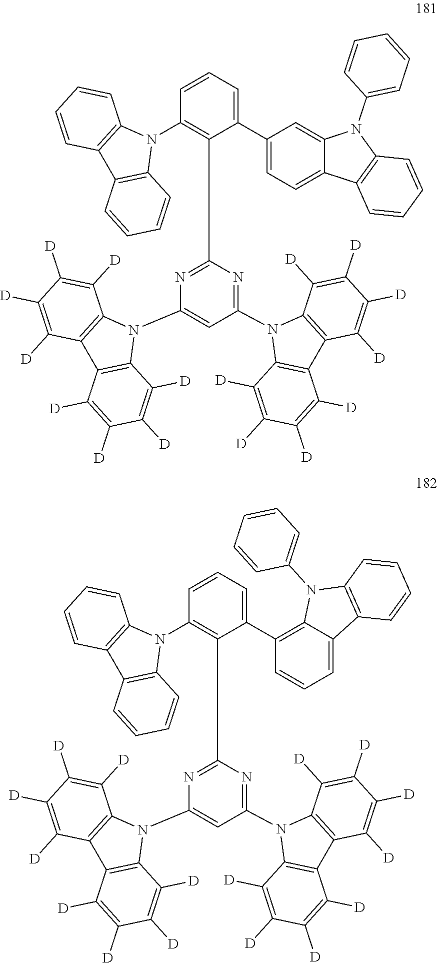 [SMILES: [2H]c1c([2H])c([2H])c2c(c1[2H])c1c([2H])c([2H])c([2H])c([2H])c1n2-c1cc(-n2c3c([2H])c([2H])c([2H])c([2H])c3c3c([2H])c([2H])c([2H])c([2H])c32)nc(-c2c(-c3ccc4c5ccccc5n(-c5ccccc5)c4c3)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3c([2H])c([2H])c([2H])c([2H])c3c3c([2H])c([2H])c([2H])c([2H])c32)nc(-c2c(-c3cccc4c5ccccc5n(-c5ccccc5)c34)cccc2-n2c3ccccc3c3ccccc32)n1] 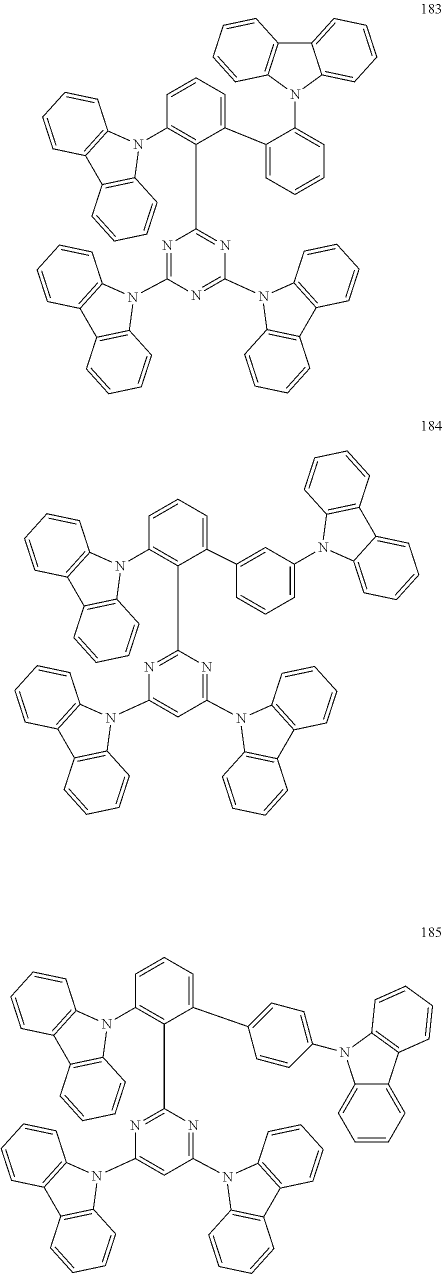 [SMILES: c1ccc(-n2c3ccccc3c3ccccc32)c(-c2cccc(-n3c4ccccc4c4ccccc43)c2-c2nc(-n3c4ccccc4c4ccccc43)nc(-n3c4ccccc4c4ccccc43)n2)c1 ; c1cc(-c2cccc(-n3c4ccccc4c4ccccc43)c2-c2nc(-n3c4ccccc4c4ccccc43)cc(-n3c4ccccc4c4ccccc43)n2)cc(-n2c3ccccc3c3ccccc32)c1 ; c1cc(-c2ccc(-n3c4ccccc4c4ccccc43)cc2)c(-c2nc(-n3c4ccccc4c4ccccc43)cc(-n3c4ccccc4c4ccccc43)n2)c(-n2c3ccccc3c3ccccc32)c1] 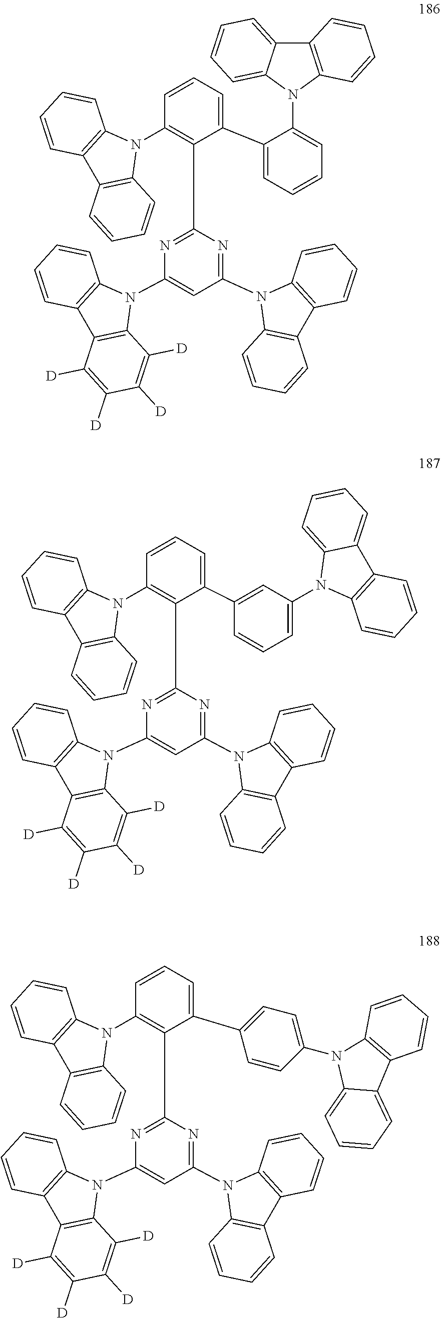 [SMILES: [2H]c1c([2H])c([2H])c2c(c1[2H])c1ccccc1n2-c1cc(-n2c3ccccc3c3ccccc32)nc(-c2c(-c3ccccc3-n3c4ccccc4c4ccccc43)cccc2-n2c3ccccc3c3ccccc32)n1 ; [2H]c1c([2H])c([2H])c2c(c1[2H])c1ccccc1n2-c1cc(-n2c3ccccc3c3ccccc32)nc(-c2c(-c3cccc(-n4c5ccccc5c5ccccc54)c3)cccc2-n2c3ccccc3c3ccccc32)n1 ; [2H]c1c([2H])c([2H])c2c(c1[2H])c1ccccc1n2-c1cc(-n2c3ccccc3c3ccccc32)nc(-c2c(-c3ccc(-n4c5ccccc5c5ccccc54)cc3)cccc2-n2c3ccccc3c3ccccc32)n1] 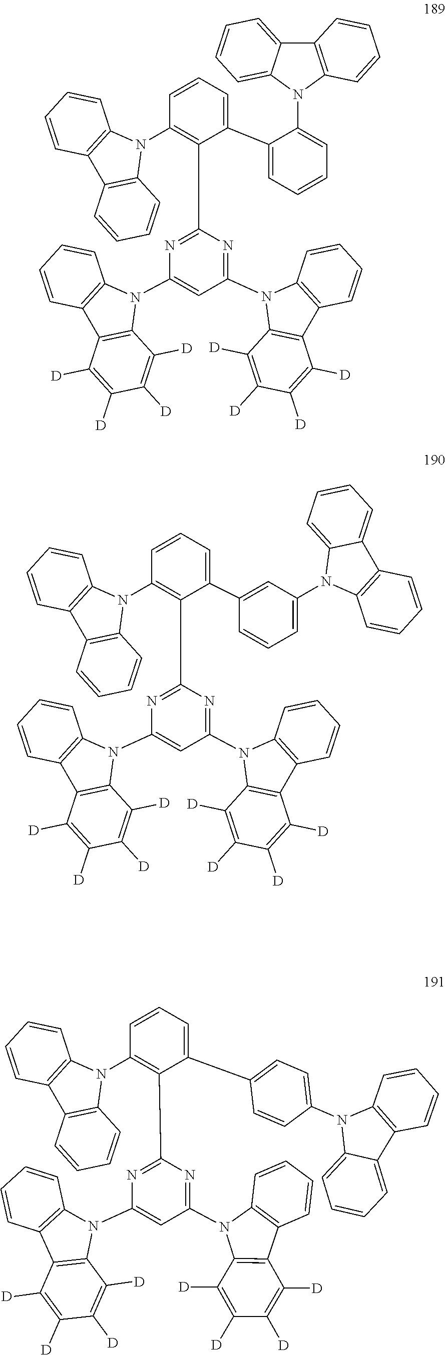 [SMILES: [2H]c1c([2H])c([2H])c2c(c1[2H])c1ccccc1n2-c1cc(-n2c3ccccc3c3c([2H])c([2H])c([2H])c([2H])c32)nc(-c2c(-c3ccccc3-n3c4ccccc4c4ccccc43)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-c3cccc(-n4c5ccccc5c5ccccc54)c3)cccc2-n2c3ccccc3c3ccccc32)n1 ; [2H]c1c([2H])c([2H])c2c(c1[2H])c1ccccc1n2-c1cc(-n2c3ccccc3c3c([2H])c([2H])c([2H])c([2H])c32)nc(-c2c(-c3ccc(-n4c5ccccc5c5ccccc54)cc3)cccc2-n2c3ccccc3c3ccccc32)n1] 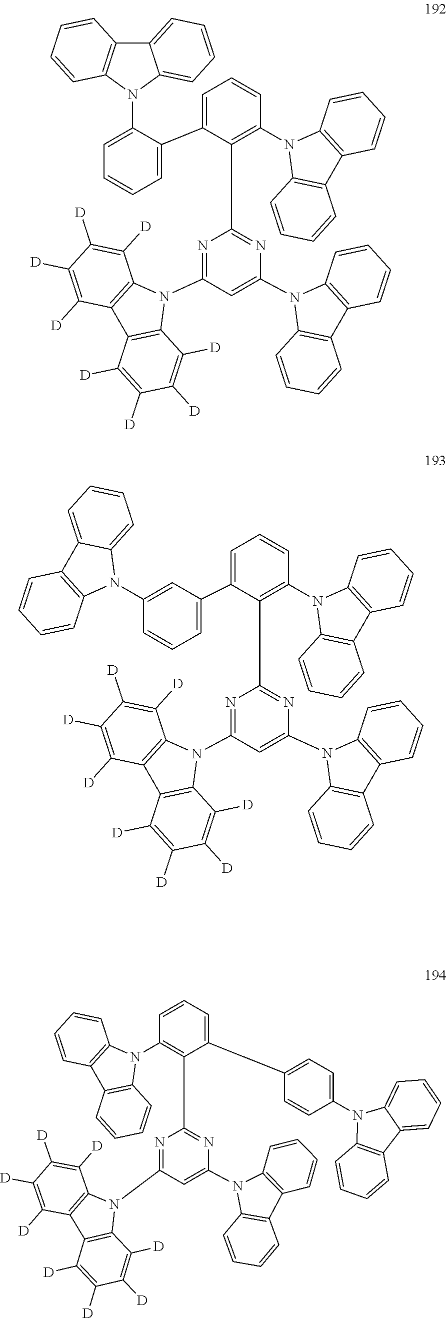 [SMILES: [2H]c1c([2H])c([2H])c2c(c1[2H])c1c([2H])c([2H])c([2H])c([2H])c1n2-c1cc(-n2c3ccccc3c3ccccc32)nc(-c2c(-c3ccccc3-n3c4ccccc4c4ccccc43)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3ccccc3c3ccccc32)nc(-c2c(-c3cccc(-n4c5ccccc5c5ccccc54)c3)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3ccccc3c3ccccc32)nc(-c2c(-c3ccc(-n4c5ccccc5c5ccccc54)cc3)cccc2-n2c3ccccc3c3ccccc32)n1] 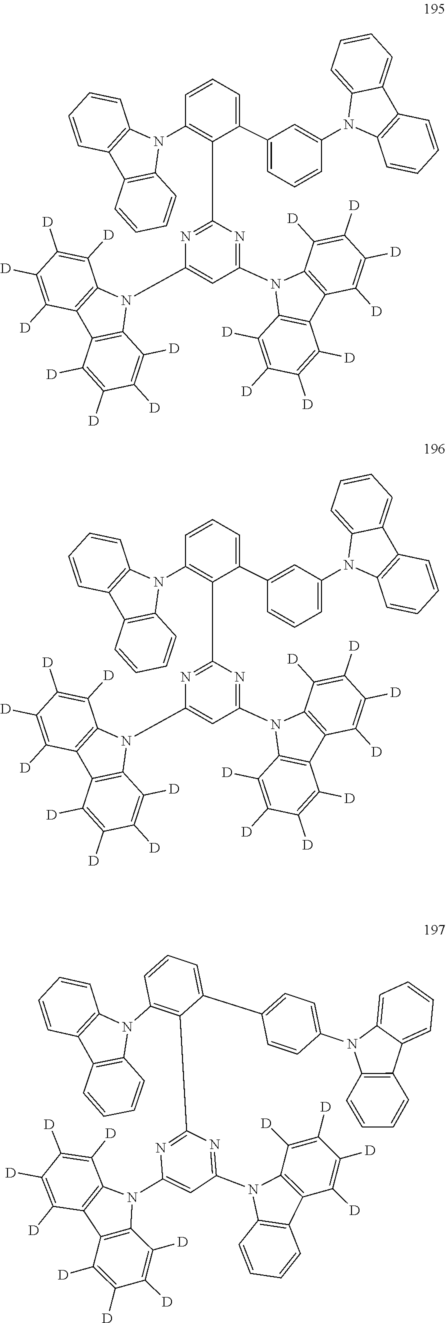 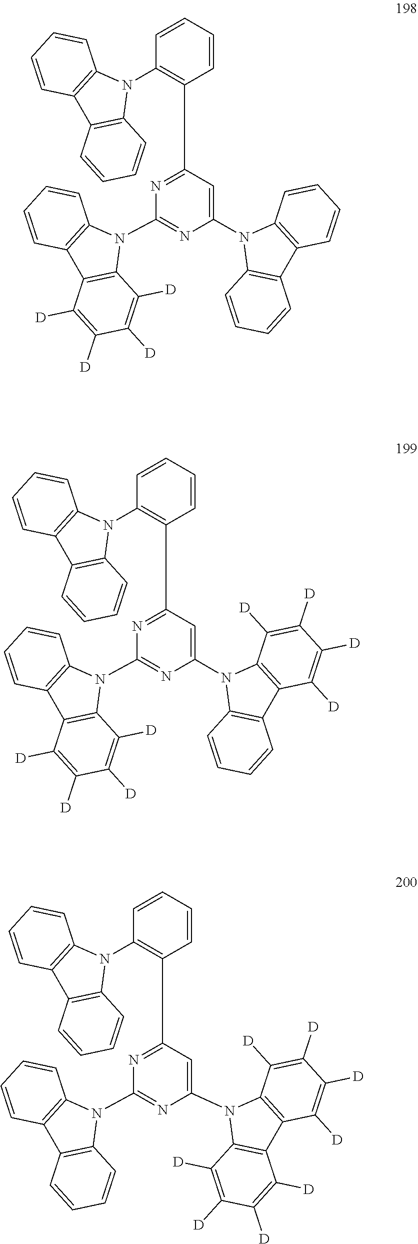 [SMILES: [2H]c1c([2H])c([2H])c2c(c1[2H])c1ccccc1n2-c1nc(-c2ccccc2-n2c3ccccc3c3ccccc32)cc(-n2c3ccccc3c3ccccc32)n1 ; [2H]c1c([2H])c([2H])c2c(c1[2H])c1ccccc1n2-c1cc(-c2ccccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1c([2H])c([2H])c([2H])c([2H])c1n2-c1cc(-c2ccccc2-n2c3ccccc3c3ccccc32)nc(-n2c3ccccc3c3ccccc32)n1] 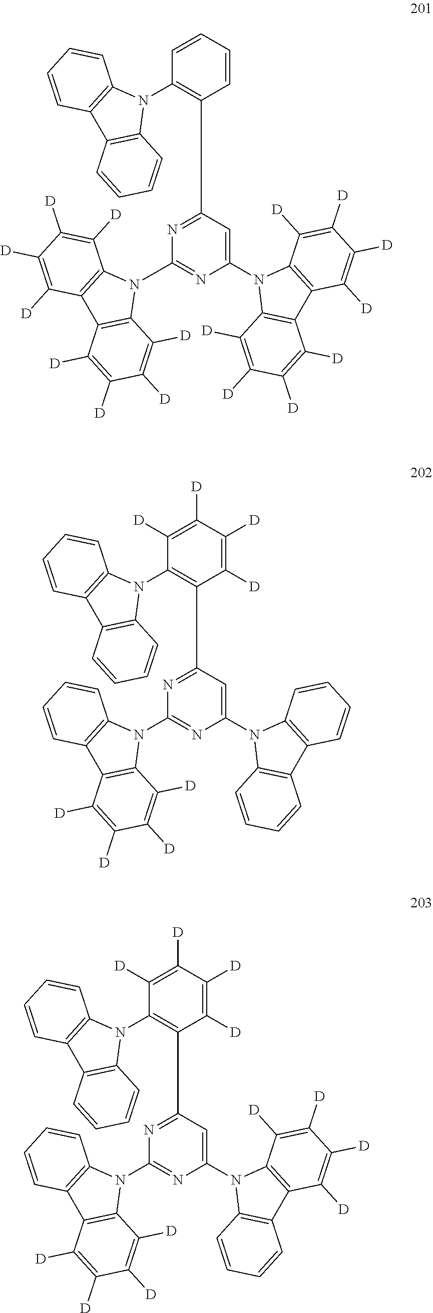 [SMILES: [2H]c1c([2H])c([2H])c2c(c1[2H])c1c([2H])c([2H])c([2H])c([2H])c1n2-c1cc(-c2ccccc2-n2c3ccccc3c3ccccc32)nc(-n2c3c([2H])c([2H])c([2H])c([2H])c3c3c([2H])c([2H])c([2H])c([2H])c32)n1 ; [2H]c1c([2H])c([2H])c(-n2c3ccccc3c3ccccc32)c(-c2cc(-n3c4ccccc4c4ccccc43)nc(-n3c4ccccc4c4c([2H])c([2H])c([2H])c([2H])c43)n2)c1[2H] ; [2H]c1c([2H])c([2H])c(-n2c3ccccc3c3ccccc32)c(-c2cc(-n3c4ccccc4c4c([2H])c([2H])c([2H])c([2H])c43)nc(-n3c4ccccc4c4c([2H])c([2H])c([2H])c([2H])c43)n2)c1[2H]] 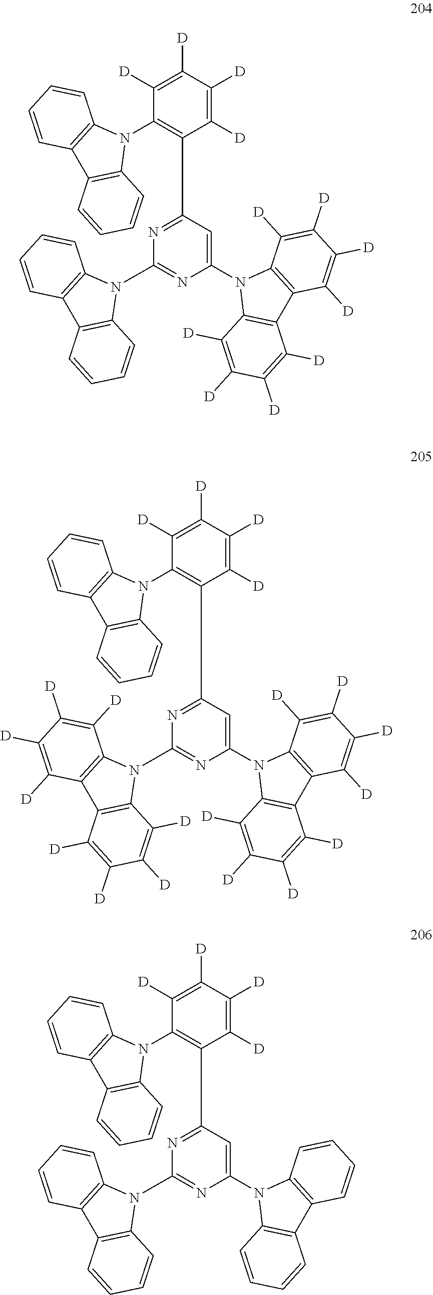 [SMILES: [2H]c1c([2H])c([2H])c(-n2c3ccccc3c3ccccc32)c(-c2cc(-n3c4c([2H])c([2H])c([2H])c([2H])c4c4c([2H])c([2H])c([2H])c([2H])c43)nc(-n3c4ccccc4c4ccccc43)n2)c1[2H] ; [2H]c1c([2H])c([2H])c(-n2c3ccccc3c3ccccc32)c(-c2cc(-n3c4c([2H])c([2H])c([2H])c([2H])c4c4c([2H])c([2H])c([2H])c([2H])c43)nc(-n3c4c([2H])c([2H])c([2H])c([2H])c4c4c([2H])c([2H])c([2H])c([2H])c43)n2)c1[2H] ; [2H]c1c([2H])c([2H])c(-n2c3ccccc3c3ccccc32)c(-c2cc(-n3c4ccccc4c4ccccc43)nc(-n3c4ccccc4c4ccccc43)n2)c1[2H]] 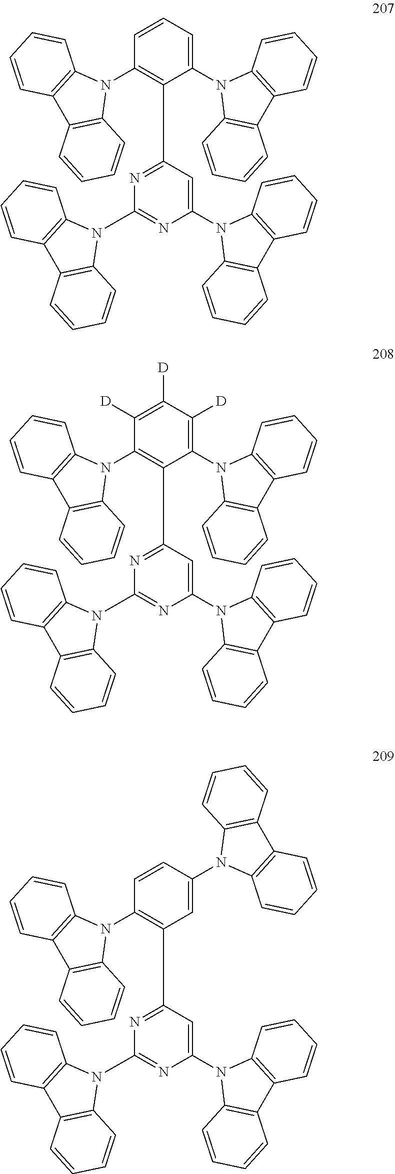 [SMILES: c1cc(-n2c3ccccc3c3ccccc32)c(-c2cc(-n3c4ccccc4c4ccccc43)nc(-n3c4ccccc4c4ccccc43)n2)c(-n2c3ccccc3c3ccccc32)c1 ; [2H]c1c([2H])c(-n2c3ccccc3c3ccccc32)c(-c2cc(-n3c4ccccc4c4ccccc43)nc(-n3c4ccccc4c4ccccc43)n2)c(-n2c3ccccc3c3ccccc32)c1[2H] ; c1ccc2c(c1)c1ccccc1n2-c1ccc(-n2c3ccccc3c3ccccc32)c(-c2cc(-n3c4ccccc4c4ccccc43)nc(-n3c4ccccc4c4ccccc43)n2)c1] 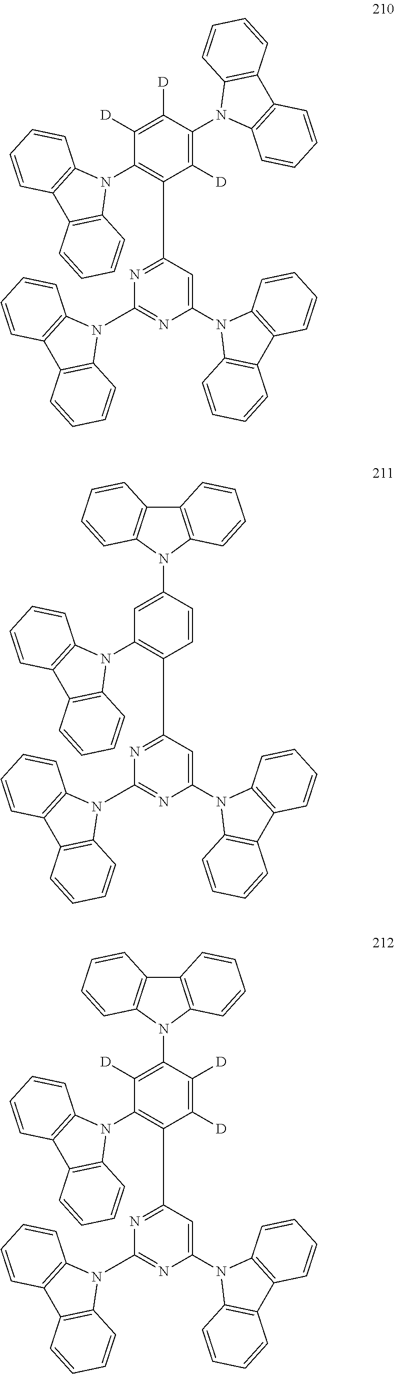 [SMILES: [2H]c1c([2H])c(-n2c3ccccc3c3ccccc32)c(-c2cc(-n3c4ccccc4c4ccccc43)nc(-n3c4ccccc4c4ccccc43)n2)c([2H])c1-n1c2ccccc2c2ccccc21 ; c1ccc2c(c1)c1ccccc1n2-c1ccc(-c2cc(-n3c4ccccc4c4ccccc43)nc(-n3c4ccccc4c4ccccc43)n2)c(-n2c3ccccc3c3ccccc32)c1 ; [2H]c1c([2H])c(-n2c3ccccc3c3ccccc32)c([2H])c(-n2c3ccccc3c3ccccc32)c1-c1cc(-n2c3ccccc3c3ccccc32)nc(-n2c3ccccc3c3ccccc32)n1] 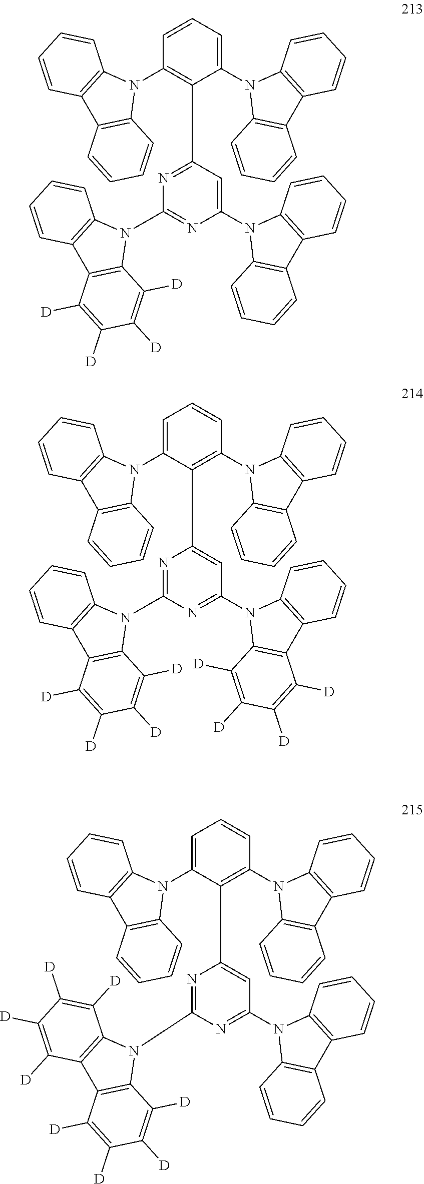 [SMILES: [2H]c1c([2H])c([2H])c2c(c1[2H])c1ccccc1n2-c1nc(-c2c(-n3c4ccccc4c4ccccc43)cccc2-n2c3ccccc3c3ccccc32)cc(-n2c3ccccc3c3ccccc32)n1 ; [2H]c1c([2H])c([2H])c2c(c1[2H])c1ccccc1n2-c1cc(-c2c(-n3c4ccccc4c4ccccc43)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1c([2H])c([2H])c([2H])c([2H])c1n2-c1nc(-c2c(-n3c4ccccc4c4ccccc43)cccc2-n2c3ccccc3c3ccccc32)cc(-n2c3ccccc3c3ccccc32)n1] 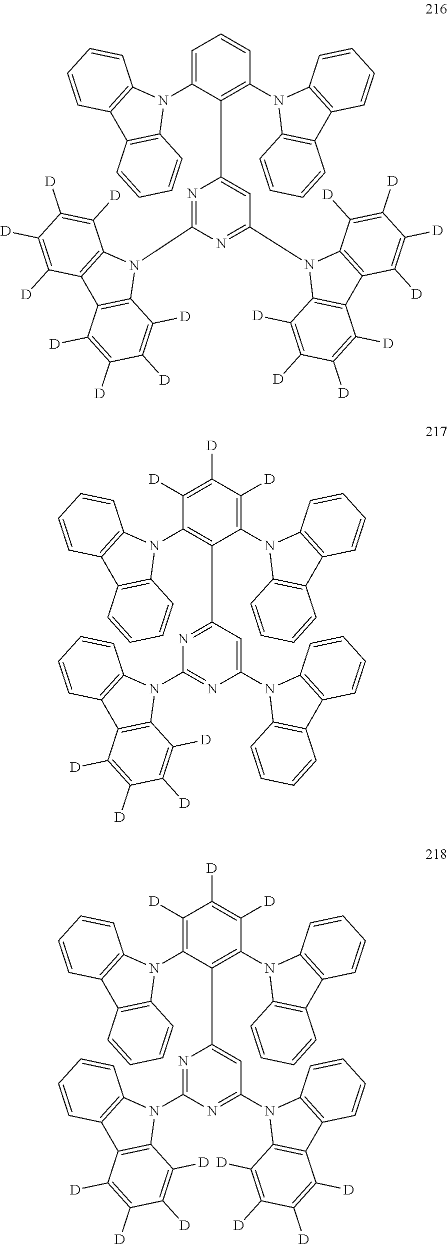 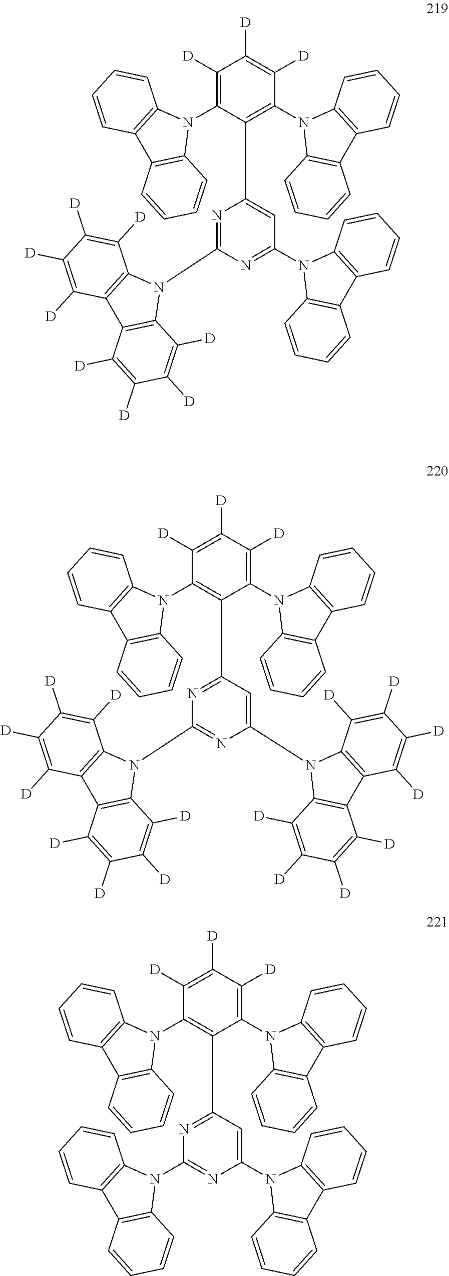 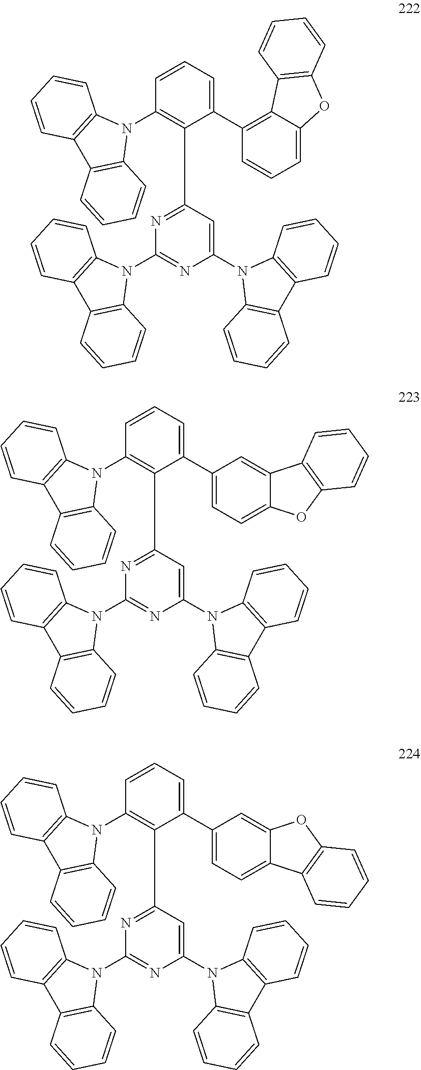 [SMILES: c1cc(-c2cccc3oc4ccccc4c23)c(-c2cc(-n3c4ccccc4c4ccccc43)nc(-n3c4ccccc4c4ccccc43)n2)c(-n2c3ccccc3c3ccccc32)c1 ; c1cc(-c2ccc3oc4ccccc4c3c2)c(-c2cc(-n3c4ccccc4c4ccccc43)nc(-n3c4ccccc4c4ccccc43)n2)c(-n2c3ccccc3c3ccccc32)c1 ; c1cc(-c2ccc3c(c2)oc2ccccc23)c(-c2cc(-n3c4ccccc4c4ccccc43)nc(-n3c4ccccc4c4ccccc43)n2)c(-n2c3ccccc3c3ccccc32)c1] 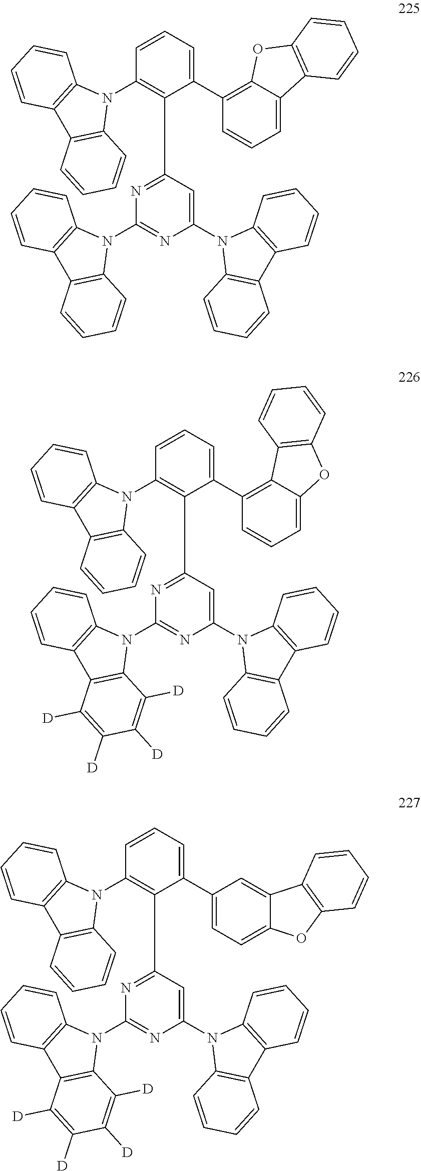 [SMILES: c1cc(-c2cccc3c2oc2ccccc23)c(-c2cc(-n3c4ccccc4c4ccccc43)nc(-n3c4ccccc4c4ccccc43)n2)c(-n2c3ccccc3c3ccccc32)c1 ; [2H]c1c([2H])c([2H])c2c(c1[2H])c1ccccc1n2-c1nc(-c2c(-c3cccc4oc5ccccc5c34)cccc2-n2c3ccccc3c3ccccc32)cc(-n2c3ccccc3c3ccccc32)n1 ; [2H]c1c([2H])c([2H])c2c(c1[2H])c1ccccc1n2-c1nc(-c2c(-c3ccc4oc5ccccc5c4c3)cccc2-n2c3ccccc3c3ccccc32)cc(-n2c3ccccc3c3ccccc32)n1] 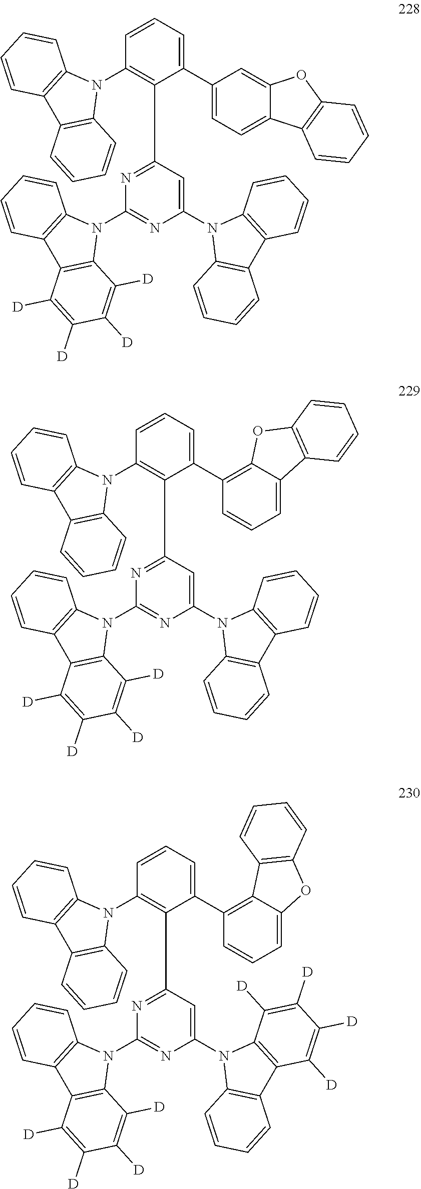 [SMILES: [2H]c1c([2H])c([2H])c2c(c1[2H])c1ccccc1n2-c1nc(-c2c(-c3ccc4c(c3)oc3ccccc34)cccc2-n2c3ccccc3c3ccccc32)cc(-n2c3ccccc3c3ccccc32)n1 ; [2H]c1c([2H])c([2H])c2c(c1[2H])c1ccccc1n2-c1nc(-c2c(-c3cccc4c3oc3ccccc34)cccc2-n2c3ccccc3c3ccccc32)cc(-n2c3ccccc3c3ccccc32)n1 ; [2H]c1c([2H])c([2H])c2c(c1[2H])c1ccccc1n2-c1cc(-c2c(-c3cccc4oc5ccccc5c34)cccc2-n2c3ccccc3c3ccccc32)nc(-n2c3ccccc3c3c([2H])c([2H])c([2H])c([2H])c32)n1] 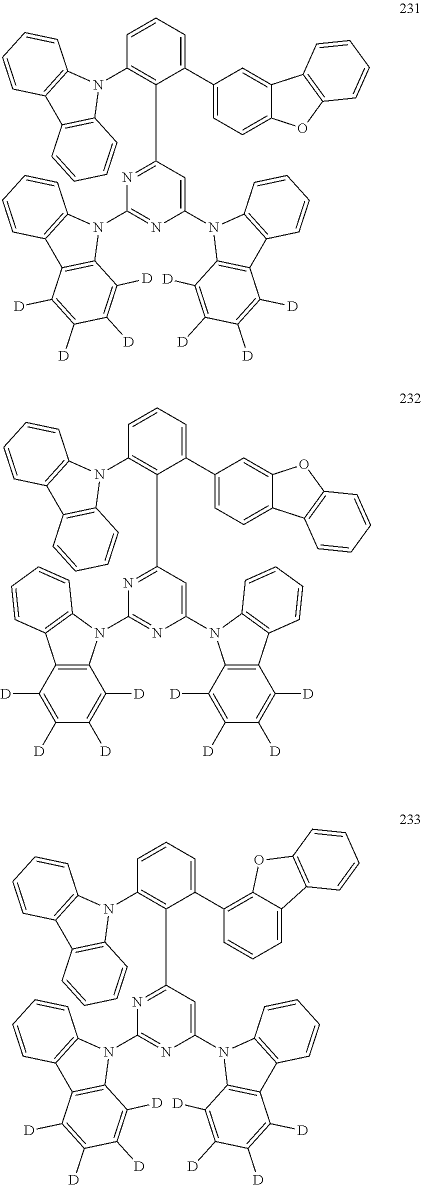 [SMILES: [2H]c1c([2H])c([2H])c2c(c1[2H])c1ccccc1n2-c1cc(-c2c(-c3ccc4oc5ccccc5c4c3)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1cc(-c2c(-c3ccc4c(c3)oc3ccccc34)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1cc(-c2c(-c3cccc4c3oc3ccccc34)cccc2-n2c3ccccc3c3ccccc32)nc(-n2c3ccccc3c3c([2H])c([2H])c([2H])c([2H])c32)n1] 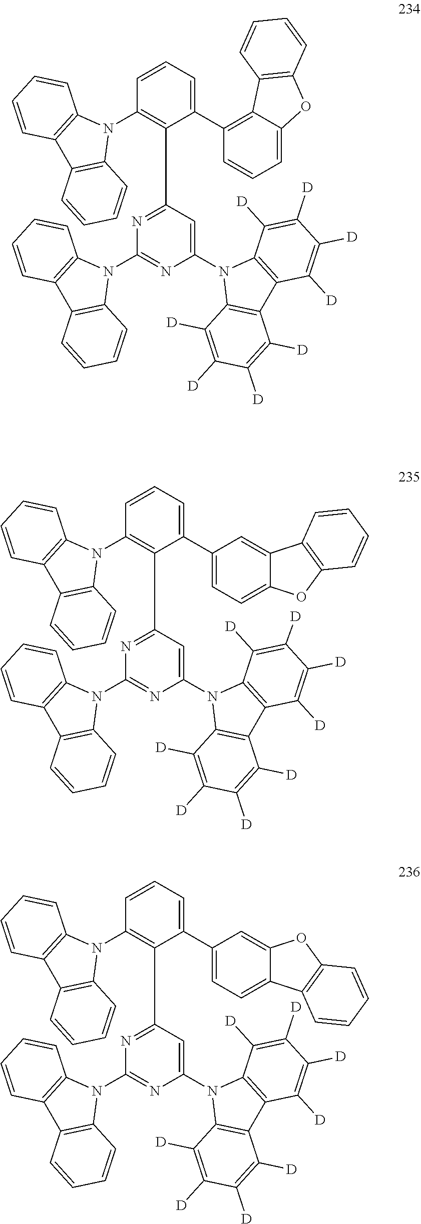 [SMILES: [2H]c1c([2H])c([2H])c2c(c1[2H])c1c([2H])c([2H])c([2H])c([2H])c1n2-c1cc(-c2c(-c3cccc4oc5ccccc5c34)cccc2-n2c3ccccc3c3ccccc32)nc(-n2c3ccccc3c3ccccc32)n1 ; [2H]c1c([2H])c([2H])c2c(c1[2H])c1c([2H])c([2H])c([2H])c([2H])c1n2-c1cc(-c2c(-c3ccc4oc5ccccc5c4c3)cccc2-n2c3ccccc3c3ccccc32)nc(-n2c3ccccc3c3ccccc32)n1 ; [2H]c1c([2H])c([2H])c2c(c1[2H])c1c([2H])c([2H])c([2H])c([2H])c1n2-c1cc(-c2c(-c3ccc4c(c3)oc3ccccc34)cccc2-n2c3ccccc3c3ccccc32)nc(-n2c3ccccc3c3ccccc32)n1] 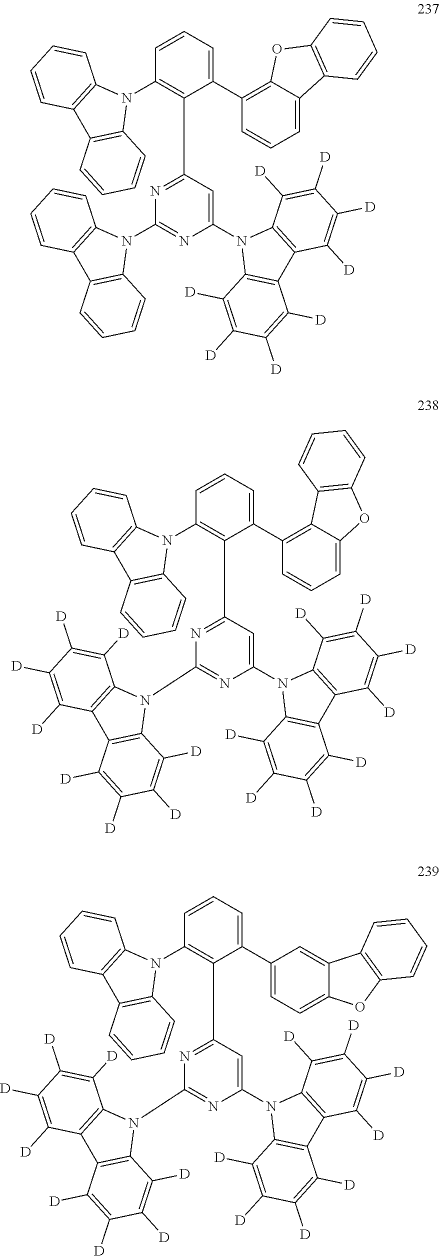 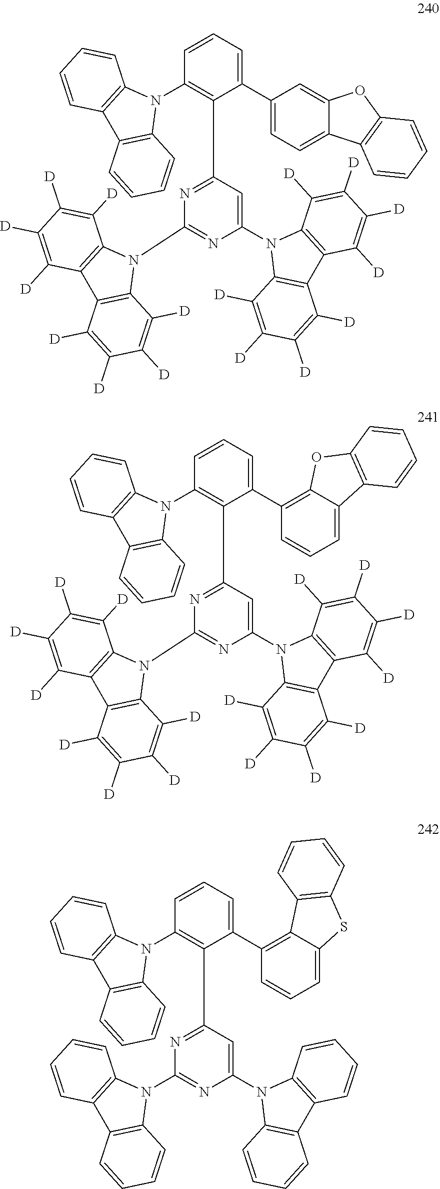 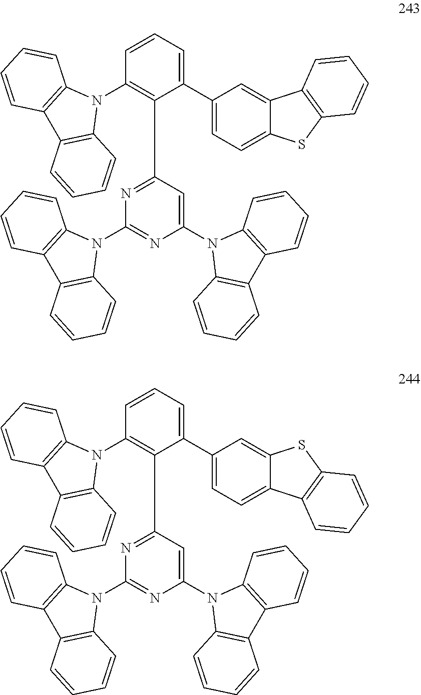 [SMILES: c1cc(-c2ccc3sc4ccccc4c3c2)c(-c2cc(-n3c4ccccc4c4ccccc43)nc(-n3c4ccccc4c4ccccc43)n2)c(-n2c3ccccc3c3ccccc32)c1 ; c1cc(-c2ccc3c(c2)sc2ccccc23)c(-c2cc(-n3c4ccccc4c4ccccc43)nc(-n3c4ccccc4c4ccccc43)n2)c(-n2c3ccccc3c3ccccc32)c1] 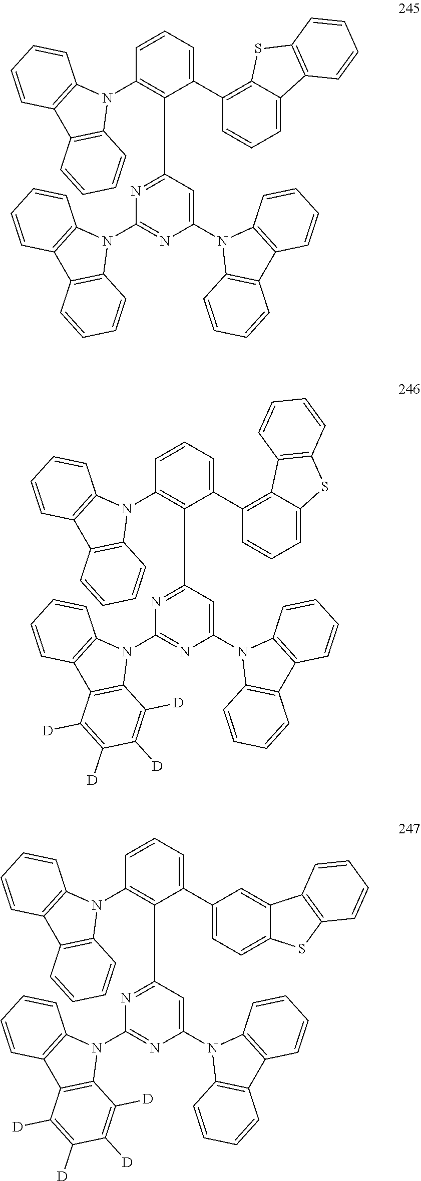 [SMILES: c1cc(-c2cccc3c2sc2ccccc23)c(-c2cc(-n3c4ccccc4c4ccccc43)nc(-n3c4ccccc4c4ccccc43)n2)c(-n2c3ccccc3c3ccccc32)c1 ; [2H]c1c([2H])c([2H])c2c(c1[2H])c1ccccc1n2-c1nc(-c2c(-c3cccc4sc5ccccc5c34)cccc2-n2c3ccccc3c3ccccc32)cc(-n2c3ccccc3c3ccccc32)n1 ; [2H]c1c([2H])c([2H])c2c(c1[2H])c1ccccc1n2-c1nc(-c2c(-c3ccc4sc5ccccc5c4c3)cccc2-n2c3ccccc3c3ccccc32)cc(-n2c3ccccc3c3ccccc32)n1] 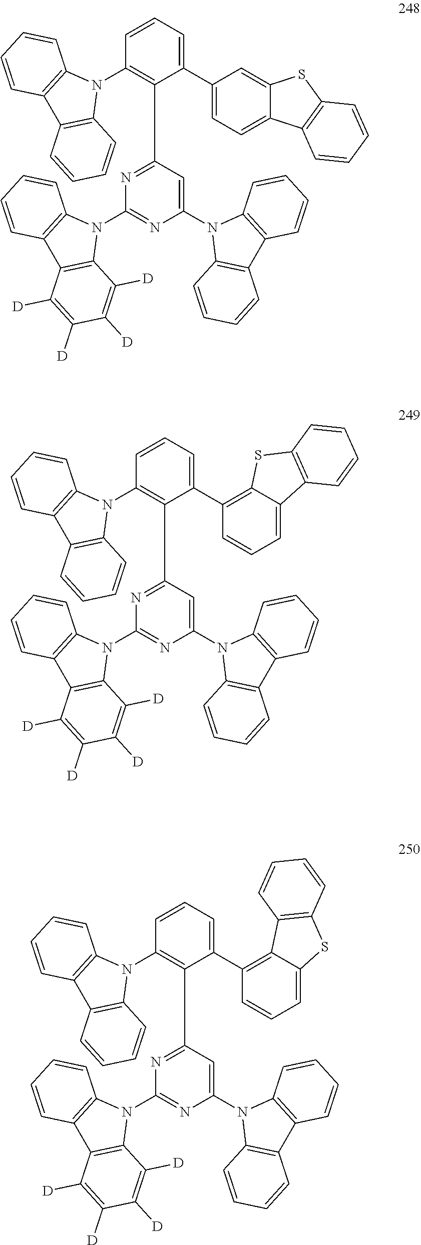 [SMILES: [2H]c1c([2H])c([2H])c2c(c1[2H])c1ccccc1n2-c1nc(-c2c(-c3ccc4c(c3)sc3ccccc34)cccc2-n2c3ccccc3c3ccccc32)cc(-n2c3ccccc3c3ccccc32)n1 ; [2H]c1c([2H])c([2H])c2c(c1[2H])c1ccccc1n2-c1nc(-c2c(-c3cccc4c3sc3ccccc34)cccc2-n2c3ccccc3c3ccccc32)cc(-n2c3ccccc3c3ccccc32)n1 ; [2H]c1c([2H])c([2H])c2c(c1[2H])c1ccccc1n2-c1nc(-c2c(-c3cccc4sc5ccccc5c34)cccc2-n2c3ccccc3c3ccccc32)cc(-n2c3ccccc3c3ccccc32)n1] 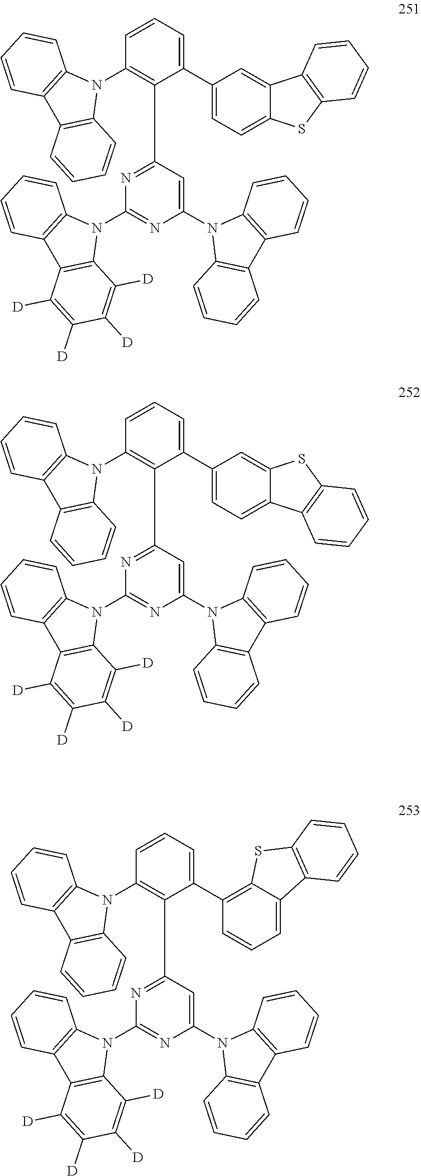 [SMILES: [2H]c1c([2H])c([2H])c2c(c1[2H])c1ccccc1n2-c1nc(-c2c(-c3ccc4sc5ccccc5c4c3)cccc2-n2c3ccccc3c3ccccc32)cc(-n2c3ccccc3c3ccccc32)n1 ; [2H]c1c([2H])c([2H])c2c(c1[2H])c1ccccc1n2-c1nc(-c2c(-c3ccc4c(c3)sc3ccccc34)cccc2-n2c3ccccc3c3ccccc32)cc(-n2c3ccccc3c3ccccc32)n1 ; [2H]c1c([2H])c([2H])c2c(c1[2H])c1ccccc1n2-c1nc(-c2c(-c3cccc4c3sc3ccccc34)cccc2-n2c3ccccc3c3ccccc32)cc(-n2c3ccccc3c3ccccc32)n1] 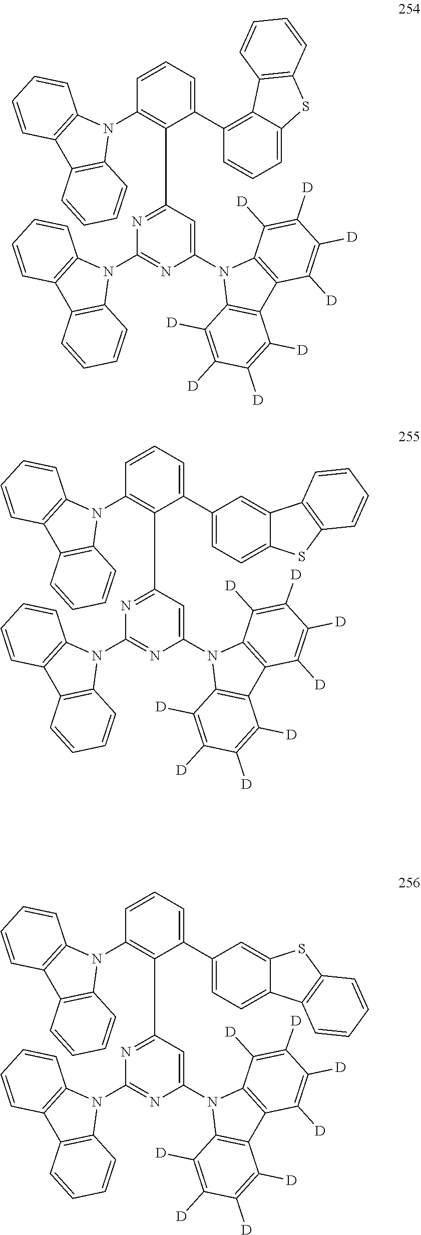 [SMILES: [2H]c1c([2H])c([2H])c2c(c1[2H])c1c([2H])c([2H])c([2H])c([2H])c1n2-c1cc(-c2c(-c3cccc4sc5ccccc5c34)cccc2-n2c3ccccc3c3ccccc32)nc(-n2c3ccccc3c3ccccc32)n1 ; [2H]c1c([2H])c([2H])c2c(c1[2H])c1c([2H])c([2H])c([2H])c([2H])c1n2-c1cc(-c2c(-c3ccc4sc5ccccc5c4c3)cccc2-n2c3ccccc3c3ccccc32)nc(-n2c3ccccc3c3ccccc32)n1 ; [2H]c1c([2H])c([2H])c2c(c1[2H])c1c([2H])c([2H])c([2H])c([2H])c1n2-c1cc(-c2c(-c3ccc4c(c3)sc3ccccc34)cccc2-n2c3ccccc3c3ccccc32)nc(-n2c3ccccc3c3ccccc32)n1] 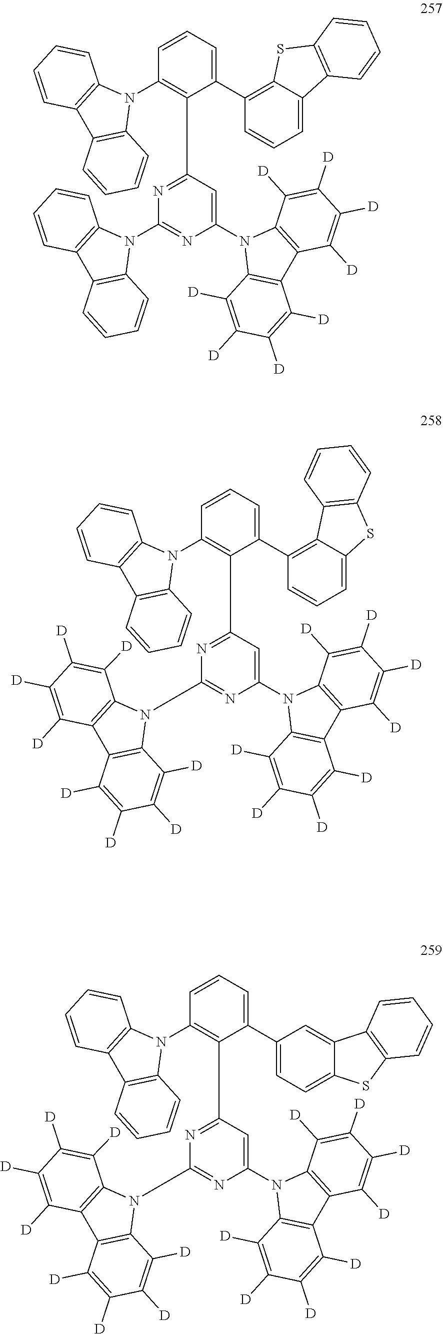 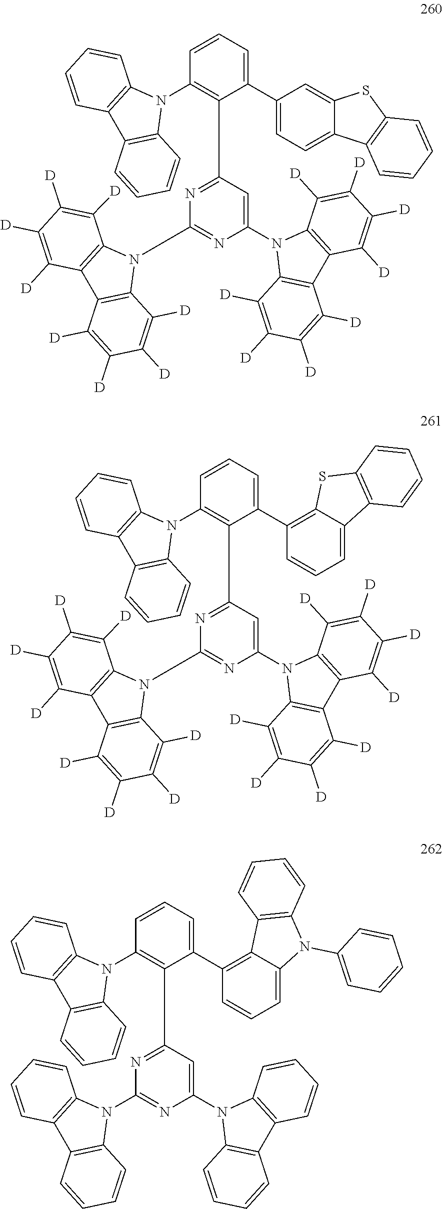 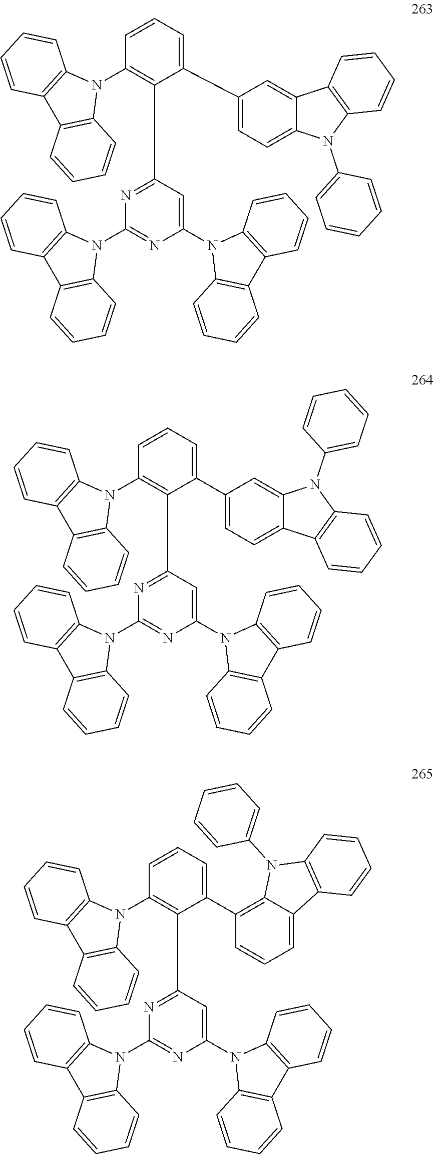 [SMILES: c1ccc(-n2c3ccccc3c3cc(-c4cccc(-n5c6ccccc6c6ccccc65)c4-c4cc(-n5c6ccccc6c6ccccc65)nc(-n5c6ccccc6c6ccccc65)n4)ccc32)cc1 ; c1ccc(-n2c3ccccc3c3ccc(-c4cccc(-n5c6ccccc6c6ccccc65)c4-c4cc(-n5c6ccccc6c6ccccc65)nc(-n5c6ccccc6c6ccccc65)n4)cc32)cc1 ; c1ccc(-n2c3ccccc3c3cccc(-c4cccc(-n5c6ccccc6c6ccccc65)c4-c4cc(-n5c6ccccc6c6ccccc65)nc(-n5c6ccccc6c6ccccc65)n4)c32)cc1] 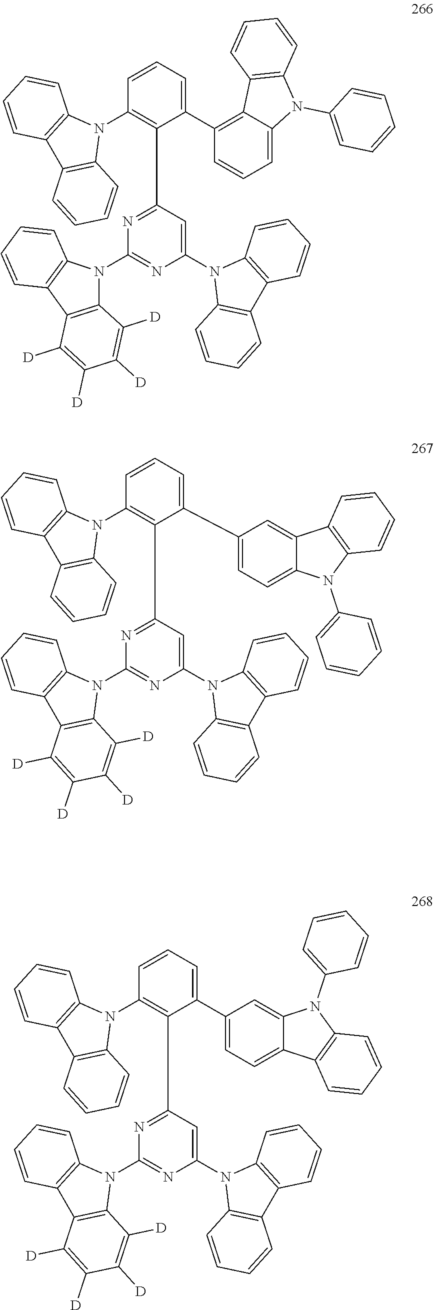 [SMILES: [2H]c1c([2H])c([2H])c2c(c1[2H])c1ccccc1n2-c1nc(-c2c(-c3cccc4c3c3ccccc3n4-c3ccccc3)cccc2-n2c3ccccc3c3ccccc32)cc(-n2c3ccccc3c3ccccc32)n1 ; [2H]c1c([2H])c([2H])c2c(c1[2H])c1ccccc1n2-c1nc(-c2c(-c3ccc4c(c3)c3ccccc3n4-c3ccccc3)cccc2-n2c3ccccc3c3ccccc32)cc(-n2c3ccccc3c3ccccc32)n1 ; [2H]c1c([2H])c([2H])c2c(c1[2H])c1ccccc1n2-c1nc(-c2c(-c3ccc4c5ccccc5n(-c5ccccc5)c4c3)cccc2-n2c3ccccc3c3ccccc32)cc(-n2c3ccccc3c3ccccc32)n1] 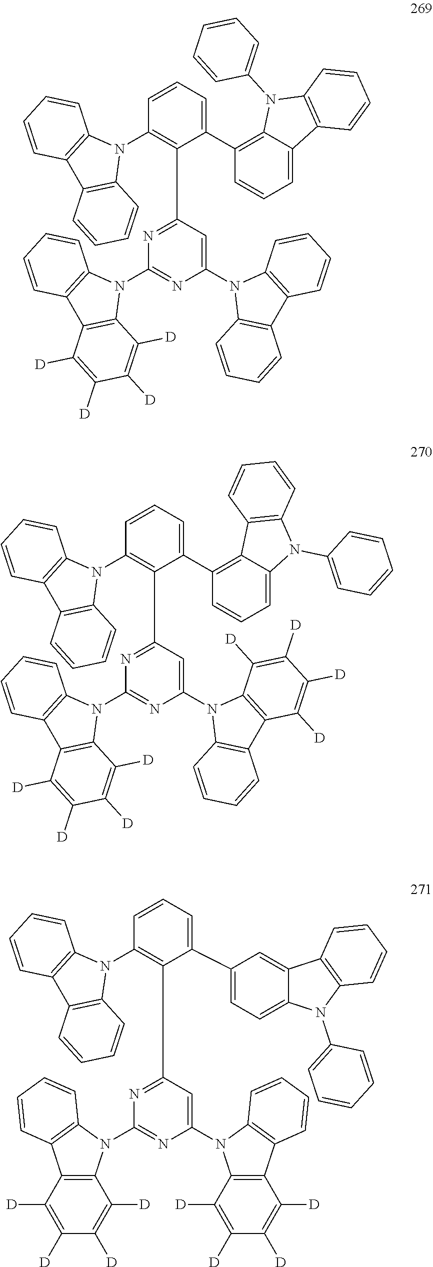 [SMILES: [2H]c1c([2H])c([2H])c2c(c1[2H])c1ccccc1n2-c1nc(-c2c(-c3cccc4c5ccccc5n(-c5ccccc5)c34)cccc2-n2c3ccccc3c3ccccc32)cc(-n2c3ccccc3c3ccccc32)n1 ; [2H]c1c([2H])c([2H])c2c(c1[2H])c1ccccc1n2-c1cc(-c2c(-c3cccc4c3c3ccccc3n4-c3ccccc3)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1cc(-c2c(-c3ccc4c(c3)c3ccccc3n4-c3ccccc3)cccc2-n2c3ccccc3c3ccccc32)nc(-n2c3ccccc3c3c([2H])c([2H])c([2H])c([2H])c32)n1] 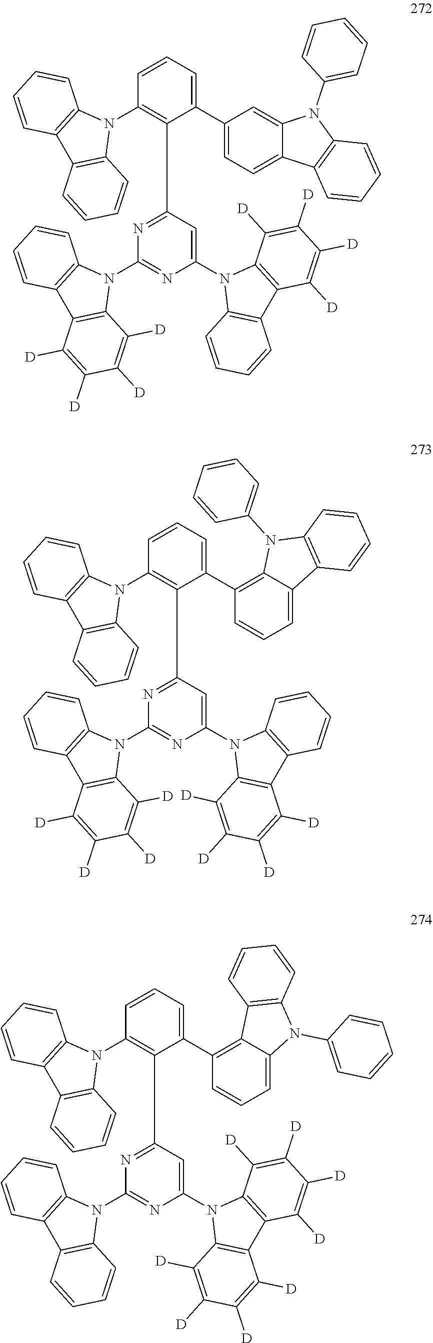 [SMILES: [2H]c1c([2H])c([2H])c2c(c1[2H])c1ccccc1n2-c1cc(-c2c(-c3ccc4c5ccccc5n(-c5ccccc5)c4c3)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1cc(-c2c(-c3cccc4c5ccccc5n(-c5ccccc5)c34)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1c([2H])c([2H])c([2H])c([2H])c1n2-c1cc(-c2c(-c3cccc4c3c3ccccc3n4-c3ccccc3)cccc2-n2c3ccccc3c3ccccc32)nc(-n2c3ccccc3c3ccccc32)n1] 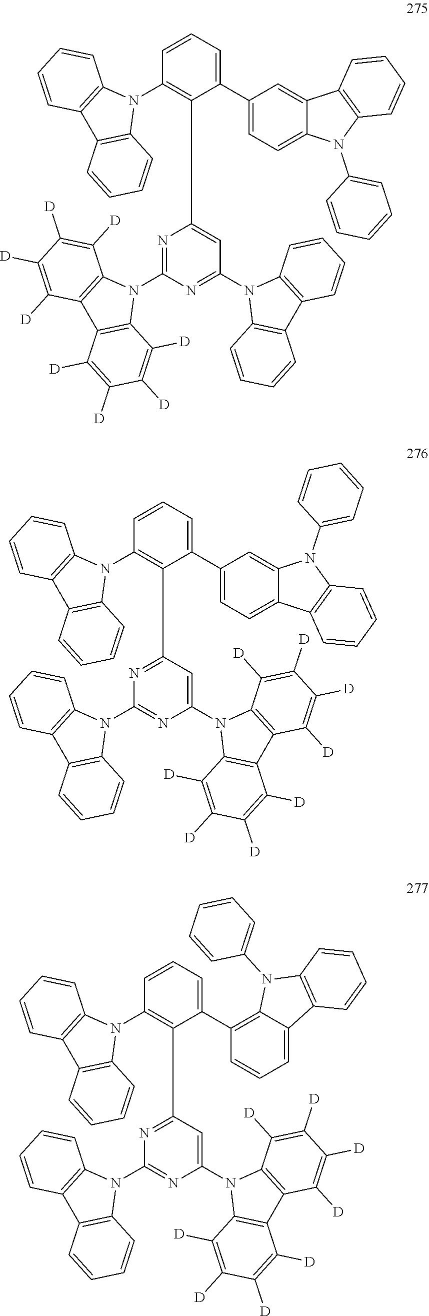 [SMILES: [2H]c1c([2H])c([2H])c2c(c1[2H])c1c([2H])c([2H])c([2H])c([2H])c1n2-c1nc(-c2c(-c3ccc4c(c3)c3ccccc3n4-c3ccccc3)cccc2-n2c3ccccc3c3ccccc32)cc(-n2c3ccccc3c3ccccc32)n1 ; [2H]c1c([2H])c([2H])c2c(c1[2H])c1c([2H])c([2H])c([2H])c([2H])c1n2-c1cc(-c2c(-c3ccc4c5ccccc5n(-c5ccccc5)c4c3)cccc2-n2c3ccccc3c3ccccc32)nc(-n2c3ccccc3c3ccccc32)n1 ; [2H]c1c([2H])c([2H])c2c(c1[2H])c1c([2H])c([2H])c([2H])c([2H])c1n2-c1cc(-c2c(-c3cccc4c5ccccc5n(-c5ccccc5)c34)cccc2-n2c3ccccc3c3ccccc32)nc(-n2c3ccccc3c3ccccc32)n1] 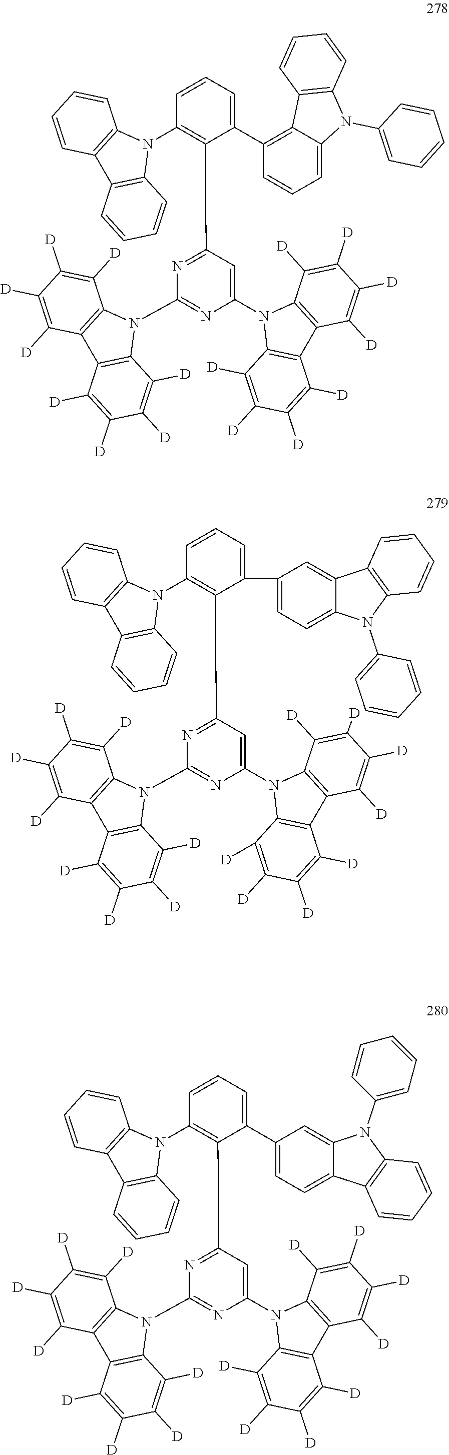 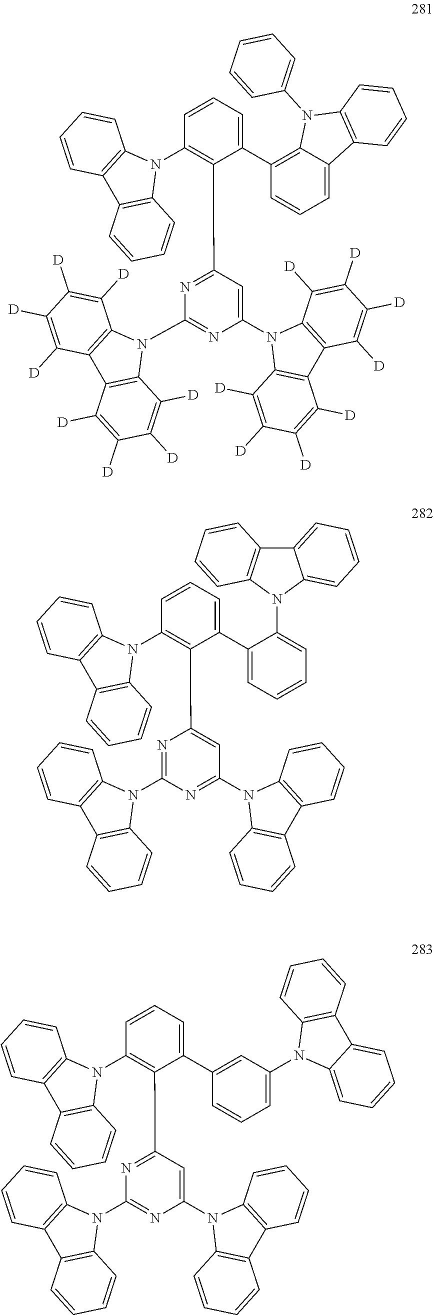 [SMILES: [2H]c1c([2H])c([2H])c2c(c1[2H])c1c([2H])c([2H])c([2H])c([2H])c1n2-c1cc(-c2c(-c3cccc4c5ccccc5n(-c5ccccc5)c34)cccc2-n2c3ccccc3c3ccccc32)nc(-n2c3c([2H])c([2H])c([2H])c([2H])c3c3c([2H])c([2H])c([2H])c([2H])c32)n1 ; c1ccc(-n2c3ccccc3c3ccccc32)c(-c2cccc(-n3c4ccccc4c4ccccc43)c2-c2cc(-n3c4ccccc4c4ccccc43)nc(-n3c4ccccc4c4ccccc43)n2)c1 ; c1cc(-c2cccc(-n3c4ccccc4c4ccccc43)c2-c2cc(-n3c4ccccc4c4ccccc43)nc(-n3c4ccccc4c4ccccc43)n2)cc(-n2c3ccccc3c3ccccc32)c1] 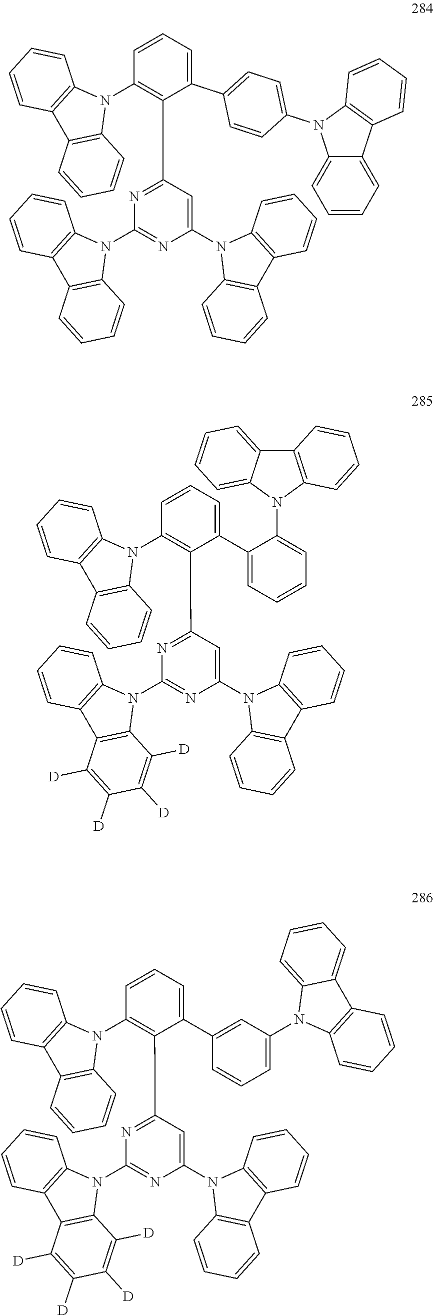 [SMILES: c1cc(-c2ccc(-n3c4ccccc4c4ccccc43)cc2)c(-c2cc(-n3c4ccccc4c4ccccc43)nc(-n3c4ccccc4c4ccccc43)n2)c(-n2c3ccccc3c3ccccc32)c1 ; [2H]c1c([2H])c([2H])c2c(c1[2H])c1ccccc1n2-c1nc(-c2c(-c3ccccc3-n3c4ccccc4c4ccccc43)cccc2-n2c3ccccc3c3ccccc32)cc(-n2c3ccccc3c3ccccc32)n1 ; [2H]c1c([2H])c([2H])c2c(c1[2H])c1ccccc1n2-c1nc(-c2c(-c3cccc(-n4c5ccccc5c5ccccc54)c3)cccc2-n2c3ccccc3c3ccccc32)cc(-n2c3ccccc3c3ccccc32)n1] 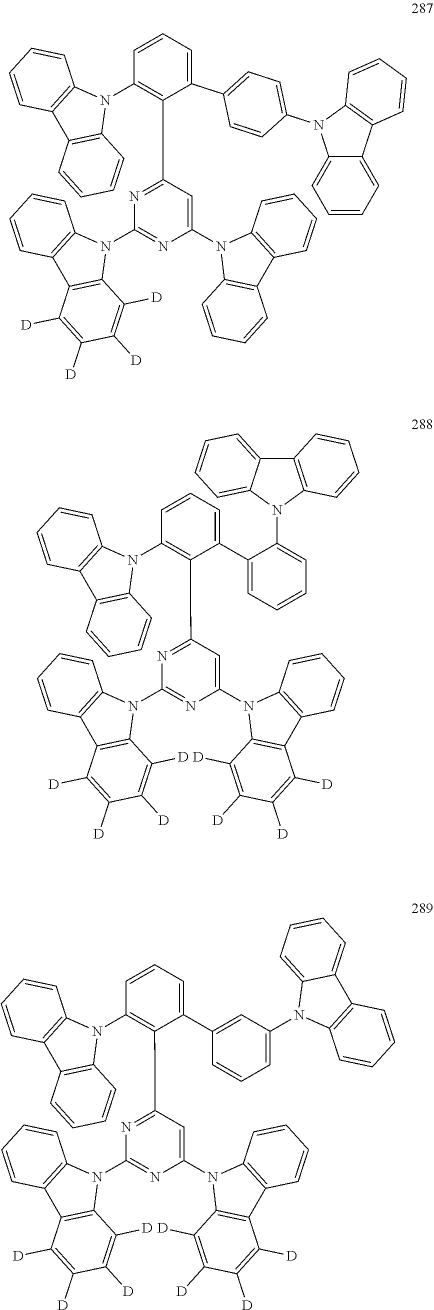 [SMILES: [2H]c1c([2H])c([2H])c2c(c1[2H])c1ccccc1n2-c1nc(-c2c(-c3ccc(-n4c5ccccc5c5ccccc54)cc3)cccc2-n2c3ccccc3c3ccccc32)cc(-n2c3ccccc3c3ccccc32)n1 ; [2H]c1c([2H])c([2H])c2c(c1[2H])c1ccccc1n2-c1cc(-c2c(-c3ccccc3-n3c4ccccc4c4ccccc43)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1cc(-c2c(-c3cccc(-n4c5ccccc5c5ccccc54)c3)cccc2-n2c3ccccc3c3ccccc32)nc(-n2c3ccccc3c3c([2H])c([2H])c([2H])c([2H])c32)n1] 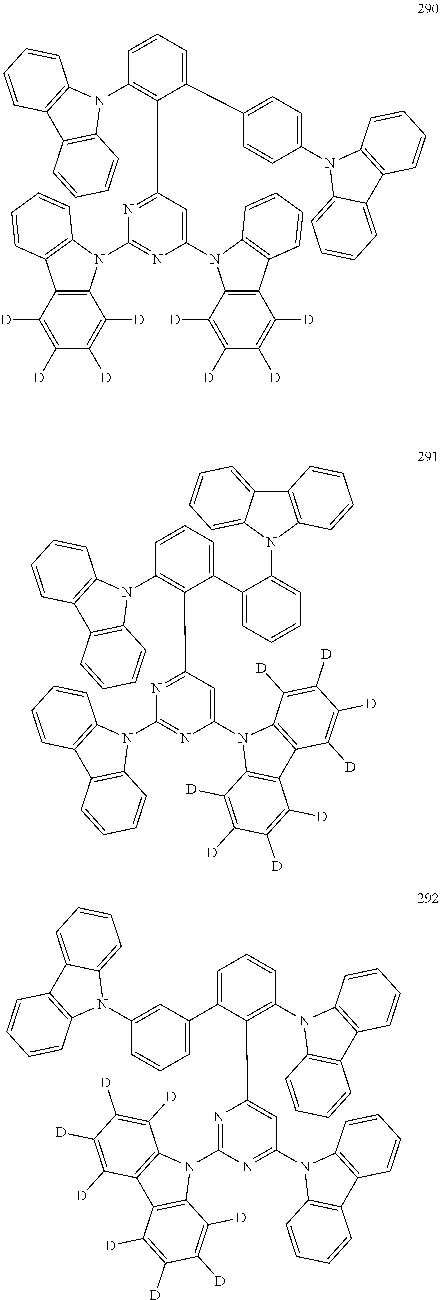 [SMILES: [2H]c1c([2H])c([2H])c2c(c1[2H])c1ccccc1n2-c1cc(-c2c(-c3ccc(-n4c5ccccc5c5ccccc54)cc3)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1c([2H])c([2H])c([2H])c([2H])c1n2-c1cc(-c2c(-c3ccccc3-n3c4ccccc4c4ccccc43)cccc2-n2c3ccccc3c3ccccc32)nc(-n2c3ccccc3c3ccccc32)n1 ; [2H]c1c([2H])c([2H])c2c(c1[2H])c1c([2H])c([2H])c([2H])c([2H])c1n2-c1nc(-c2c(-c3cccc(-n4c5ccccc5c5ccccc54)c3)cccc2-n2c3ccccc3c3ccccc32)cc(-n2c3ccccc3c3ccccc32)n1] 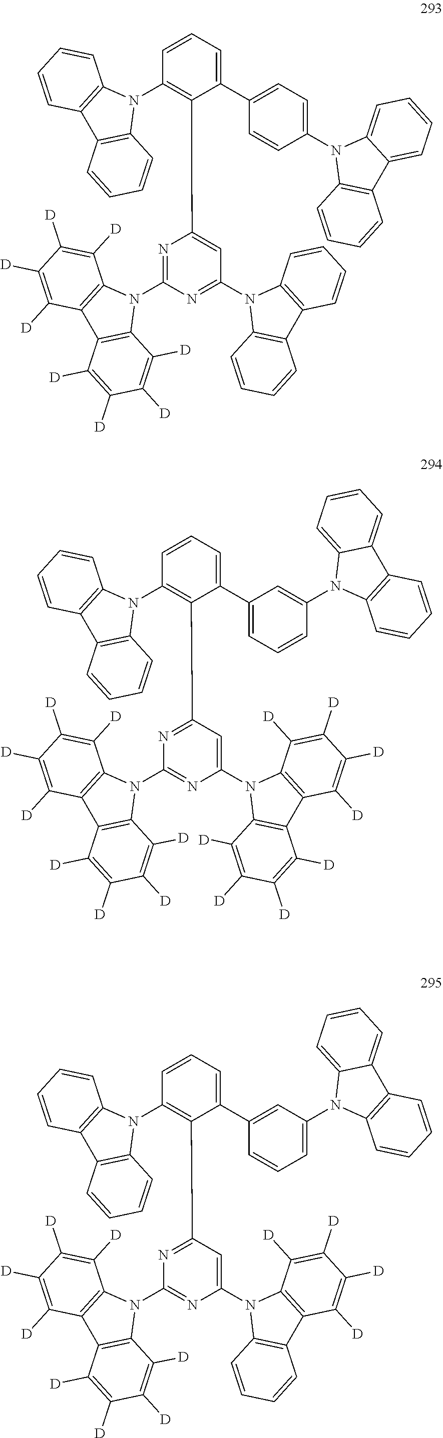 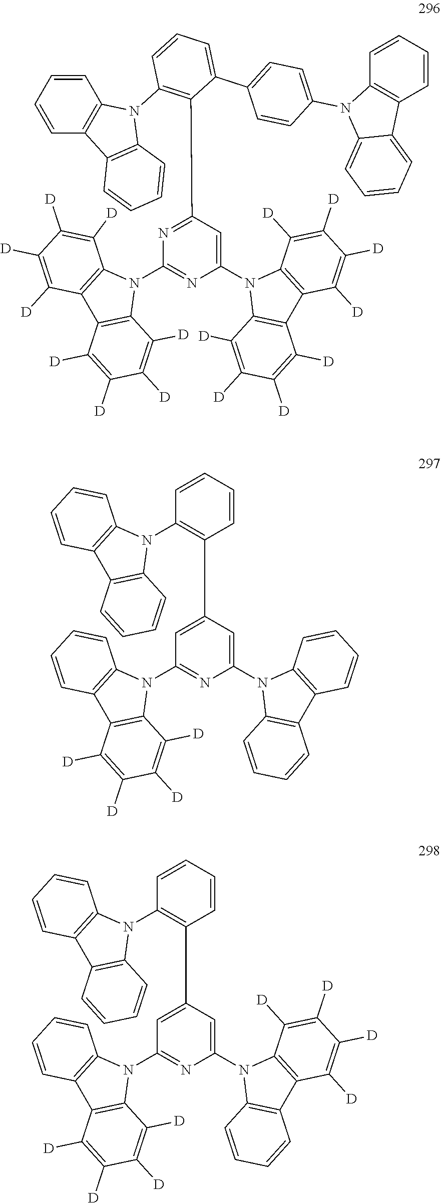 [SMILES: [2H]c1c([2H])c([2H])c2c(c1[2H])c1c([2H])c([2H])c([2H])c([2H])c1n2-c1cc(-c2c(-c3ccc(-n4c5ccccc5c5ccccc54)cc3)cccc2-n2c3ccccc3c3ccccc32)nc(-n2c3c([2H])c([2H])c([2H])c([2H])c3c3c([2H])c([2H])c([2H])c([2H])c32)n1 ; [2H]c1c([2H])c([2H])c2c(c1[2H])c1ccccc1n2-c1cc(-c2ccccc2-n2c3ccccc3c3ccccc32)cc(-n2c3ccccc3c3ccccc32)n1 ; [2H]c1c([2H])c([2H])c2c(c1[2H])c1ccccc1n2-c1cc(-c2ccccc2-n2c3ccccc3c3ccccc32)cc(-n2c3ccccc3c3c([2H])c([2H])c([2H])c([2H])c32)n1] 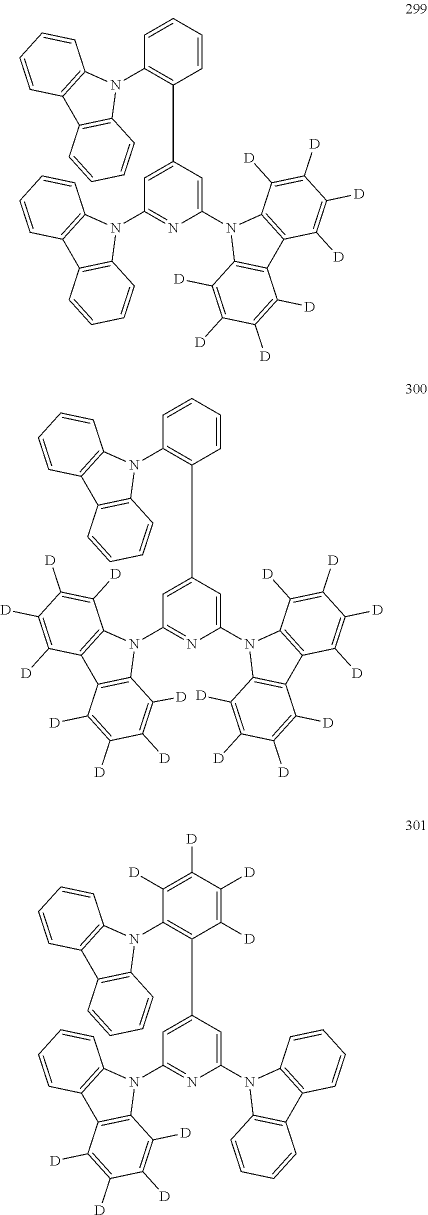 [SMILES: [2H]c1c([2H])c([2H])c2c(c1[2H])c1c([2H])c([2H])c([2H])c([2H])c1n2-c1cc(-c2ccccc2-n2c3ccccc3c3ccccc32)cc(-n2c3ccccc3c3ccccc32)n1 ; [2H]c1c([2H])c([2H])c2c(c1[2H])c1c([2H])c([2H])c([2H])c([2H])c1n2-c1cc(-c2ccccc2-n2c3ccccc3c3ccccc32)cc(-n2c3c([2H])c([2H])c([2H])c([2H])c3c3c([2H])c([2H])c([2H])c([2H])c32)n1 ; [2H]c1c([2H])c([2H])c(-n2c3ccccc3c3ccccc32)c(-c2cc(-n3c4ccccc4c4ccccc43)nc(-n3c4ccccc4c4c([2H])c([2H])c([2H])c([2H])c43)c2)c1[2H]] 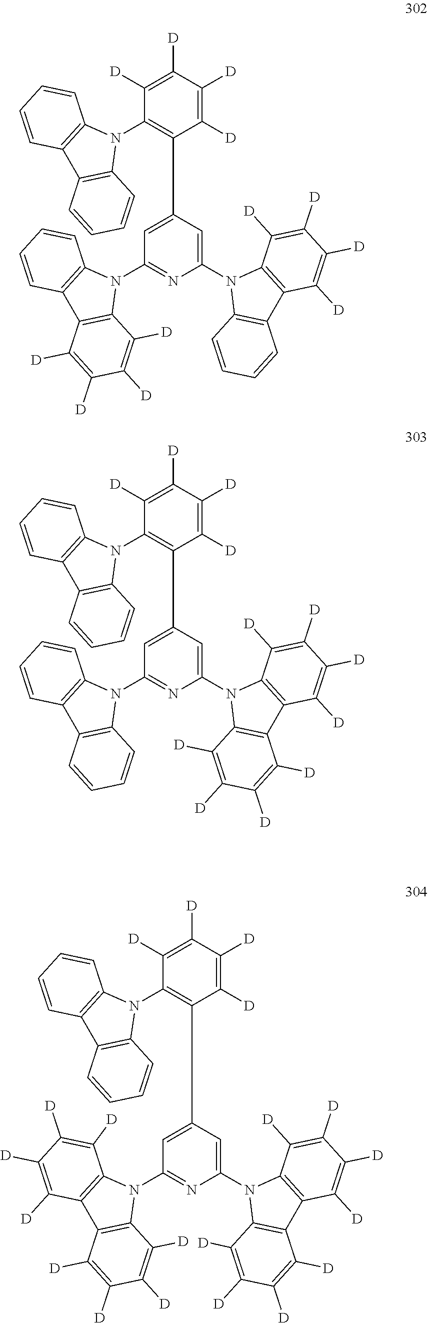 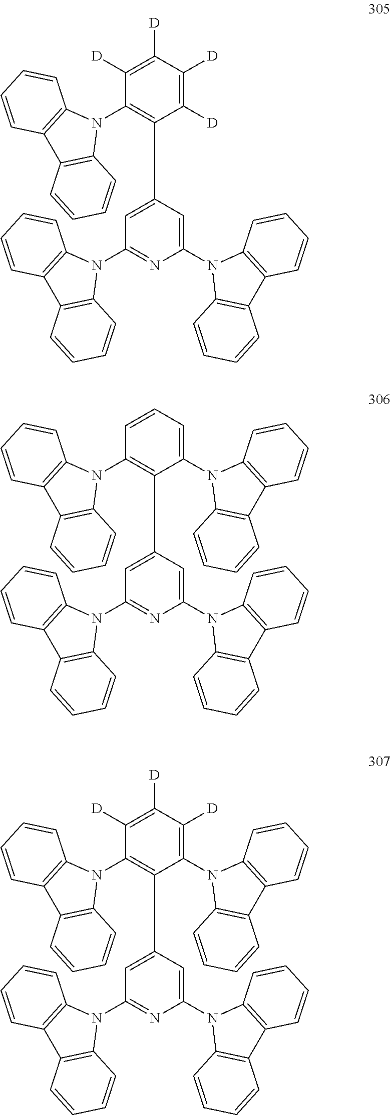 [SMILES: [2H]c1c([2H])c([2H])c(-n2c3ccccc3c3ccccc32)c(-c2cc(-n3c4ccccc4c4ccccc43)nc(-n3c4ccccc4c4ccccc43)c2)c1[2H] ; c1cc(-n2c3ccccc3c3ccccc32)c(-c2cc(-n3c4ccccc4c4ccccc43)nc(-n3c4ccccc4c4ccccc43)c2)c(-n2c3ccccc3c3ccccc32)c1 ; [2H]c1c([2H])c(-n2c3ccccc3c3ccccc32)c(-c2cc(-n3c4ccccc4c4ccccc43)nc(-n3c4ccccc4c4ccccc43)c2)c(-n2c3ccccc3c3ccccc32)c1[2H]] 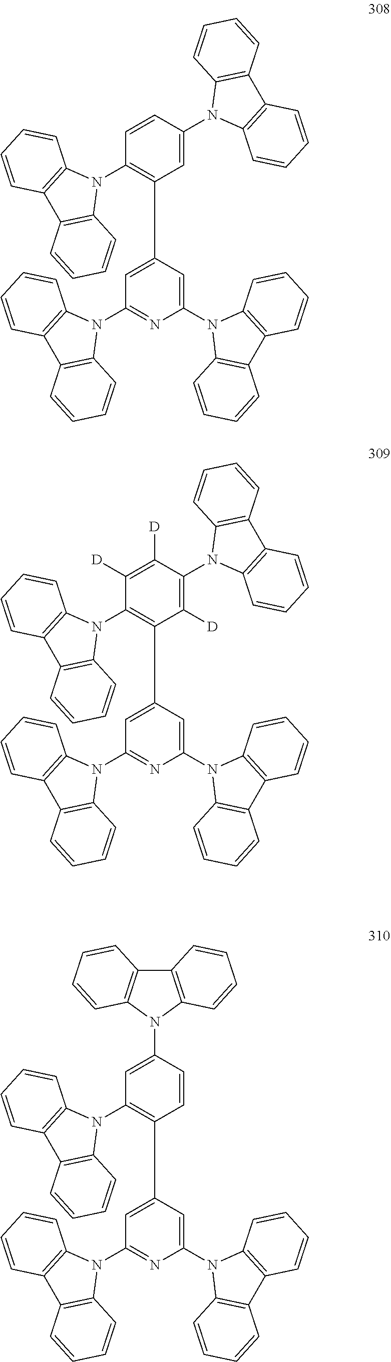 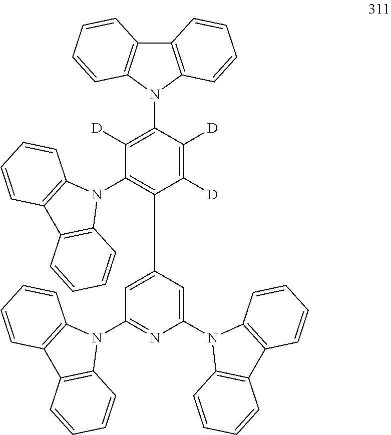 [SMILES: [2H]c1c([2H])c(-n2c3ccccc3c3ccccc32)c([2H])c(-n2c3ccccc3c3ccccc32)c1-c1cc(-n2c3ccccc3c3ccccc32)nc(-n2c3ccccc3c3ccccc32)c1] 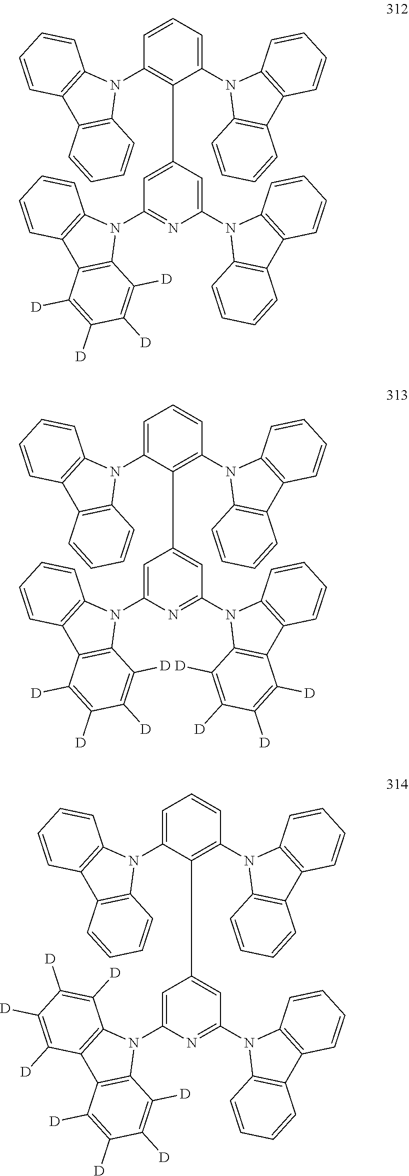 [SMILES: [2H]c1c([2H])c([2H])c2c(c1[2H])c1ccccc1n2-c1cc(-c2c(-n3c4ccccc4c4ccccc43)cccc2-n2c3ccccc3c3ccccc32)cc(-n2c3ccccc3c3ccccc32)n1 ; [2H]c1c([2H])c([2H])c2c(c1[2H])c1ccccc1n2-c1cc(-c2c(-n3c4ccccc4c4ccccc43)cccc2-n2c3ccccc3c3ccccc32)cc(-n2c3ccccc3c3c([2H])c([2H])c([2H])c([2H])c32)n1 ; [2H]c1c([2H])c([2H])c2c(c1[2H])c1c([2H])c([2H])c([2H])c([2H])c1n2-c1cc(-c2c(-n3c4ccccc4c4ccccc43)cccc2-n2c3ccccc3c3ccccc32)cc(-n2c3ccccc3c3ccccc32)n1] 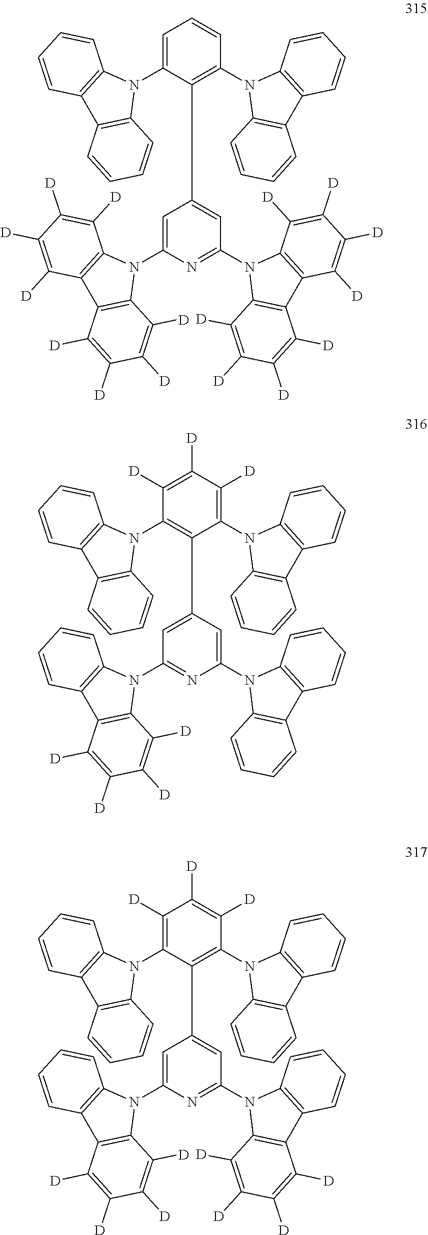 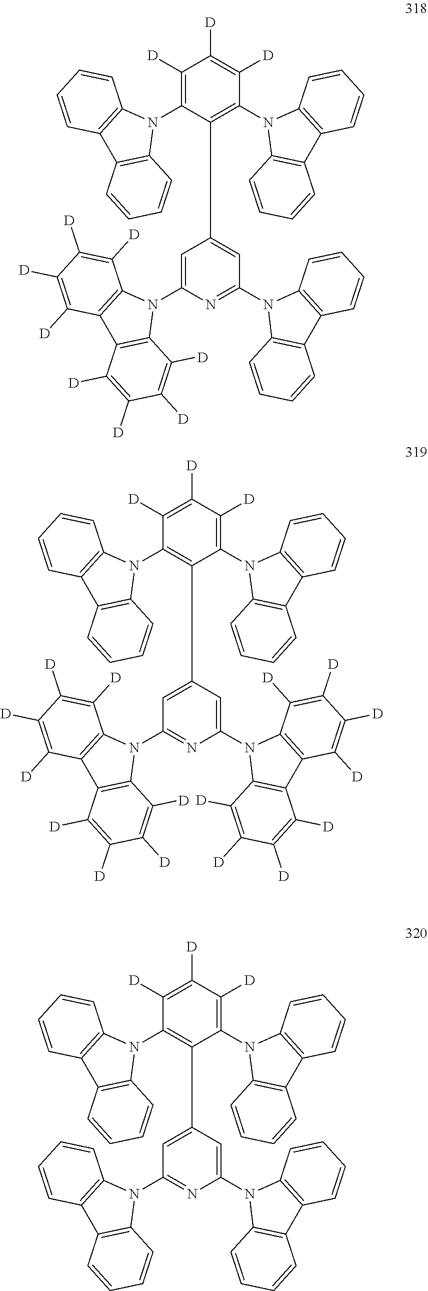 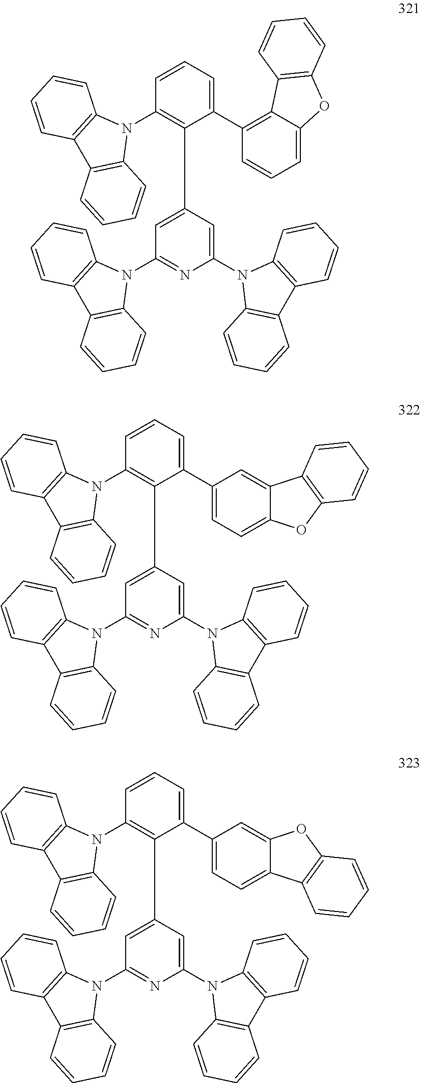 [SMILES: c1cc(-c2cccc3oc4ccccc4c23)c(-c2cc(-n3c4ccccc4c4ccccc43)nc(-n3c4ccccc4c4ccccc43)c2)c(-n2c3ccccc3c3ccccc32)c1 ; c1cc(-c2ccc3oc4ccccc4c3c2)c(-c2cc(-n3c4ccccc4c4ccccc43)nc(-n3c4ccccc4c4ccccc43)c2)c(-n2c3ccccc3c3ccccc32)c1 ; c1cc(-c2ccc3c(c2)oc2ccccc23)c(-c2cc(-n3c4ccccc4c4ccccc43)nc(-n3c4ccccc4c4ccccc43)c2)c(-n2c3ccccc3c3ccccc32)c1] 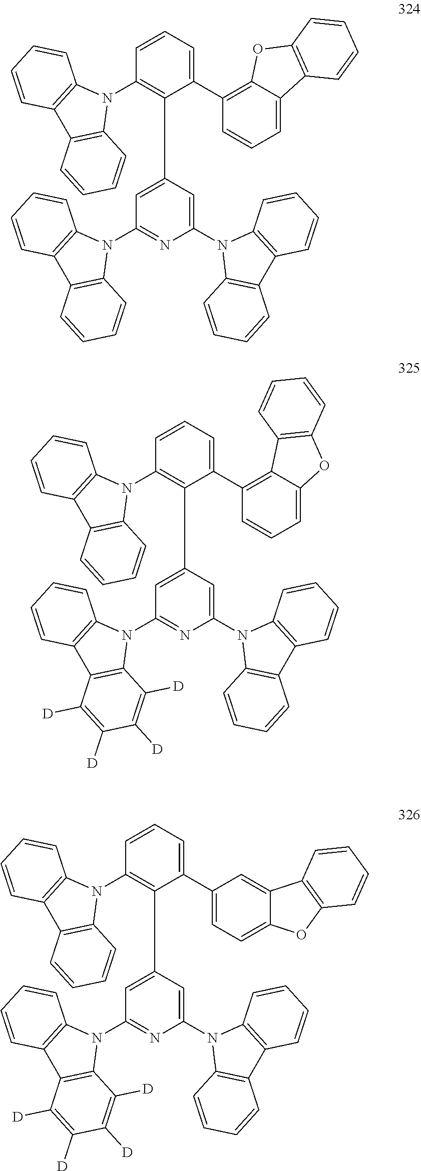 [SMILES: c1cc(-c2cccc3c2oc2ccccc23)c(-c2cc(-n3c4ccccc4c4ccccc43)nc(-n3c4ccccc4c4ccccc43)c2)c(-n2c3ccccc3c3ccccc32)c1 ; [2H]c1c([2H])c([2H])c2c(c1[2H])c1ccccc1n2-c1cc(-c2c(-c3cccc4oc5ccccc5c34)cccc2-n2c3ccccc3c3ccccc32)cc(-n2c3ccccc3c3ccccc32)n1 ; [2H]c1c([2H])c([2H])c2c(c1[2H])c1ccccc1n2-c1cc(-c2c(-c3ccc4oc5ccccc5c4c3)cccc2-n2c3ccccc3c3ccccc32)cc(-n2c3ccccc3c3ccccc32)n1] 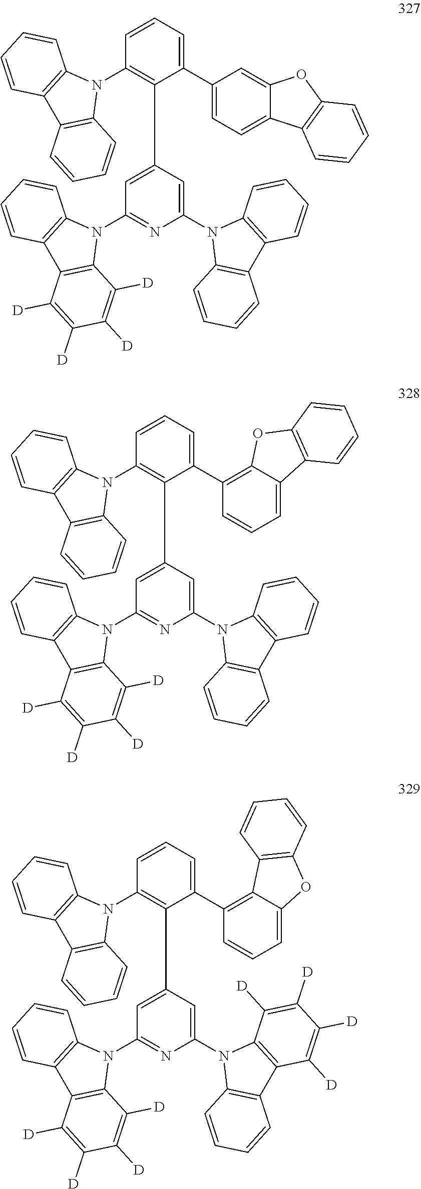 [SMILES: [2H]c1c([2H])c([2H])c2c(c1[2H])c1ccccc1n2-c1cc(-c2c(-c3ccc4c(c3)oc3ccccc34)cccc2-n2c3ccccc3c3ccccc32)cc(-n2c3ccccc3c3ccccc32)n1 ; [2H]c1c([2H])c([2H])c2c(c1[2H])c1ccccc1n2-c1cc(-c2c(-c3cccc4c3oc3ccccc34)cccc2-n2c3ccccc3c3ccccc32)cc(-n2c3ccccc3c3ccccc32)n1 ; [2H]c1c([2H])c([2H])c2c(c1[2H])c1ccccc1n2-c1cc(-c2c(-c3cccc4oc5ccccc5c34)cccc2-n2c3ccccc3c3ccccc32)cc(-n2c3ccccc3c3c([2H])c([2H])c([2H])c([2H])c32)n1] 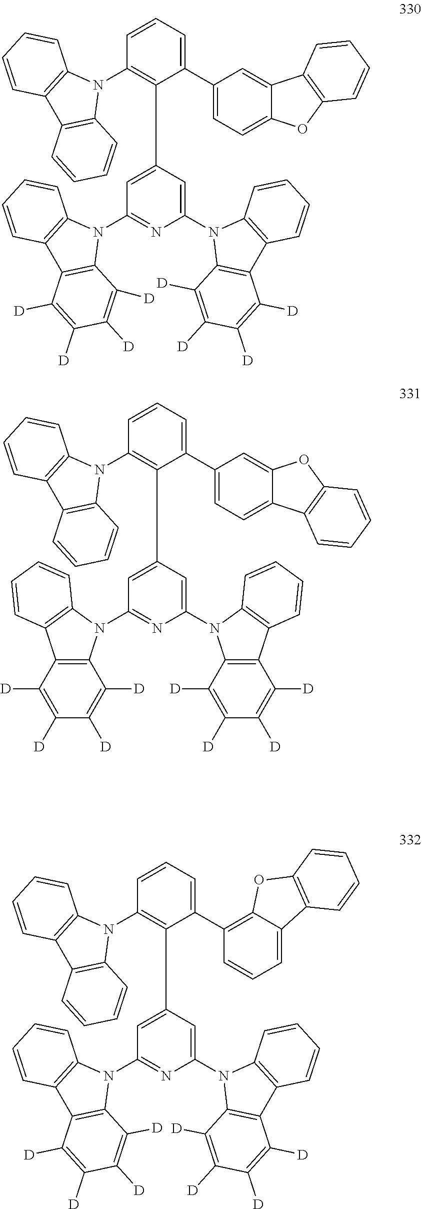 [SMILES: [2H]c1c([2H])c([2H])c2c(c1[2H])c1ccccc1n2-c1cc(-c2c(-c3ccc4oc5ccccc5c4c3)cccc2-n2c3ccccc3c3ccccc32)cc(-n2c3ccccc3c3c([2H])c([2H])c([2H])c([2H])c32)n1 ; [2H]c1c([2H])c([2H])c2c(c1[2H])c1ccccc1n2-c1cc(-c2c(-c3ccc4c(c3)oc3ccccc34)cccc2-n2c3ccccc3c3ccccc32)cc(-n2c3ccccc3c3c([2H])c([2H])c([2H])c([2H])c32)n1 ; [2H]c1c([2H])c([2H])c2c(c1[2H])c1ccccc1n2-c1cc(-c2c(-c3cccc4c3oc3ccccc34)cccc2-n2c3ccccc3c3ccccc32)cc(-n2c3ccccc3c3c([2H])c([2H])c([2H])c([2H])c32)n1] 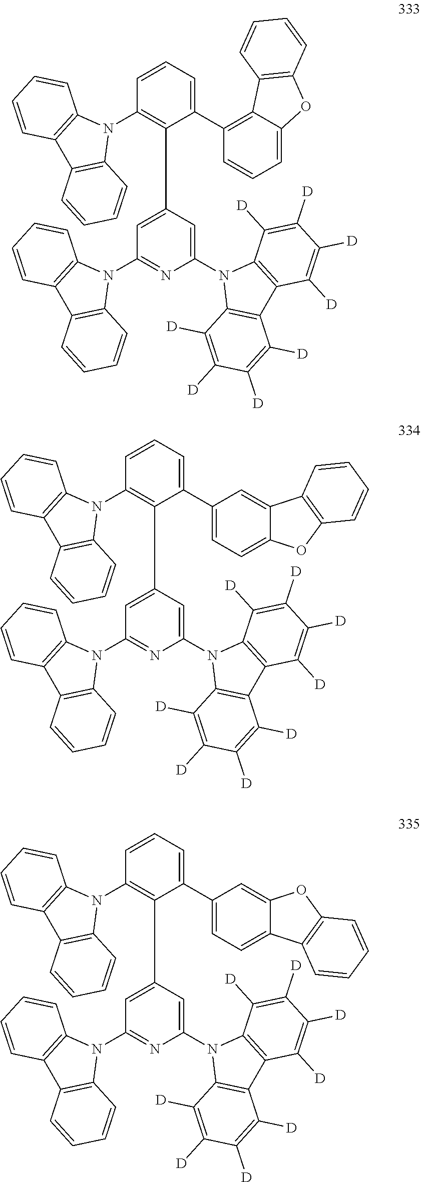 [SMILES: [2H]c1c([2H])c([2H])c2c(c1[2H])c1c([2H])c([2H])c([2H])c([2H])c1n2-c1cc(-c2c(-c3cccc4oc5ccccc5c34)cccc2-n2c3ccccc3c3ccccc32)cc(-n2c3ccccc3c3ccccc32)n1 ; [2H]c1c([2H])c([2H])c2c(c1[2H])c1c([2H])c([2H])c([2H])c([2H])c1n2-c1cc(-c2c(-c3ccc4oc5ccccc5c4c3)cccc2-n2c3ccccc3c3ccccc32)cc(-n2c3ccccc3c3ccccc32)n1 ; [2H]c1c([2H])c([2H])c2c(c1[2H])c1c([2H])c([2H])c([2H])c([2H])c1n2-c1cc(-c2c(-c3ccc4c(c3)oc3ccccc34)cccc2-n2c3ccccc3c3ccccc32)cc(-n2c3ccccc3c3ccccc32)n1] 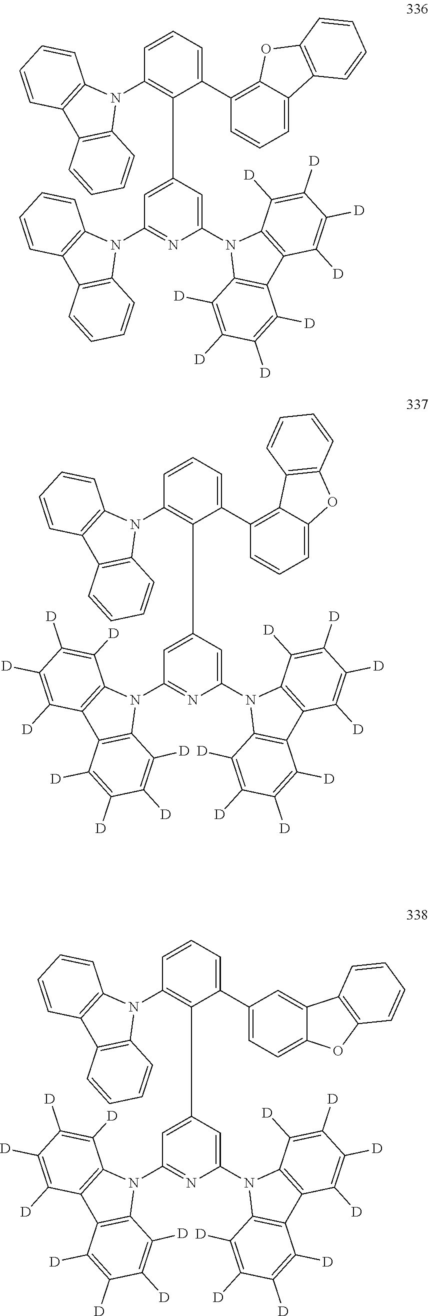 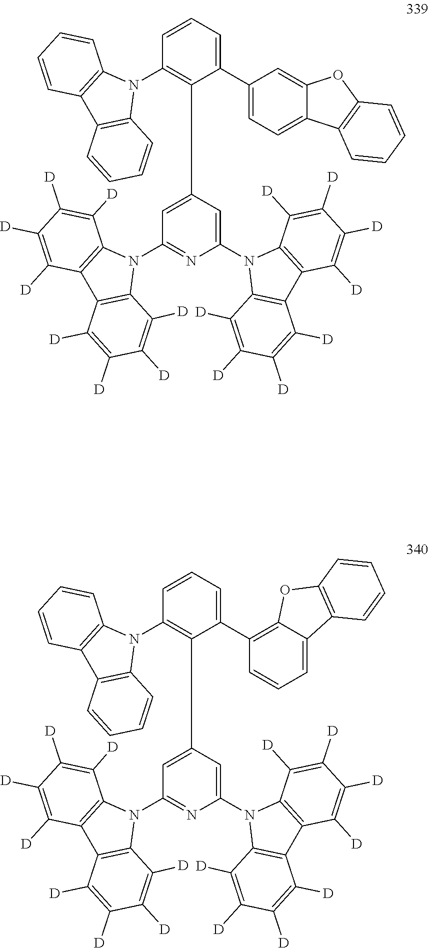 [SMILES: [2H]c1c([2H])c([2H])c2c(c1[2H])c1c([2H])c([2H])c([2H])c([2H])c1n2-c1cc(-c2c(-c3ccc4c(c3)oc3ccccc34)cccc2-n2c3ccccc3c3ccccc32)cc(-n2c3c([2H])c([2H])c([2H])c([2H])c3c3c([2H])c([2H])c([2H])c([2H])c32)n1 ; [2H]c1c([2H])c([2H])c2c(c1[2H])c1c([2H])c([2H])c([2H])c([2H])c1n2-c1cc(-c2c(-c3cccc4c3oc3ccccc34)cccc2-n2c3ccccc3c3ccccc32)cc(-n2c3c([2H])c([2H])c([2H])c([2H])c3c3c([2H])c([2H])c([2H])c([2H])c32)n1] 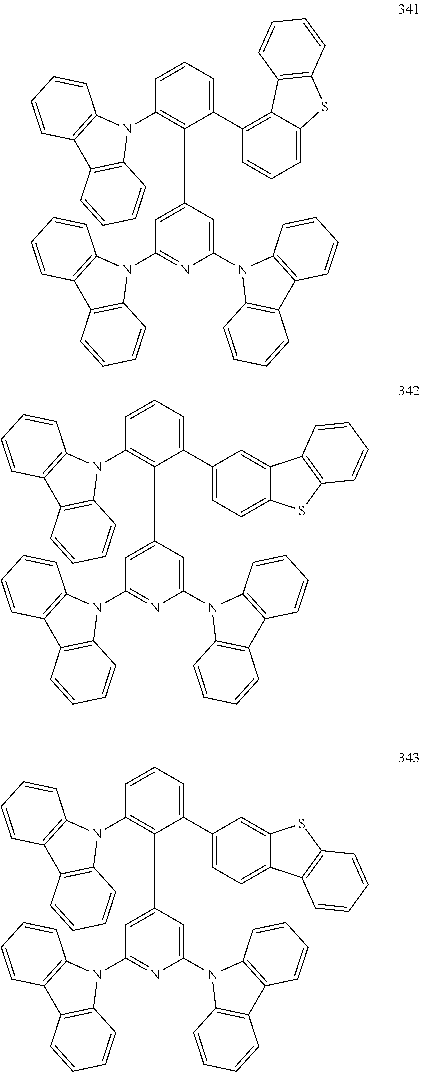 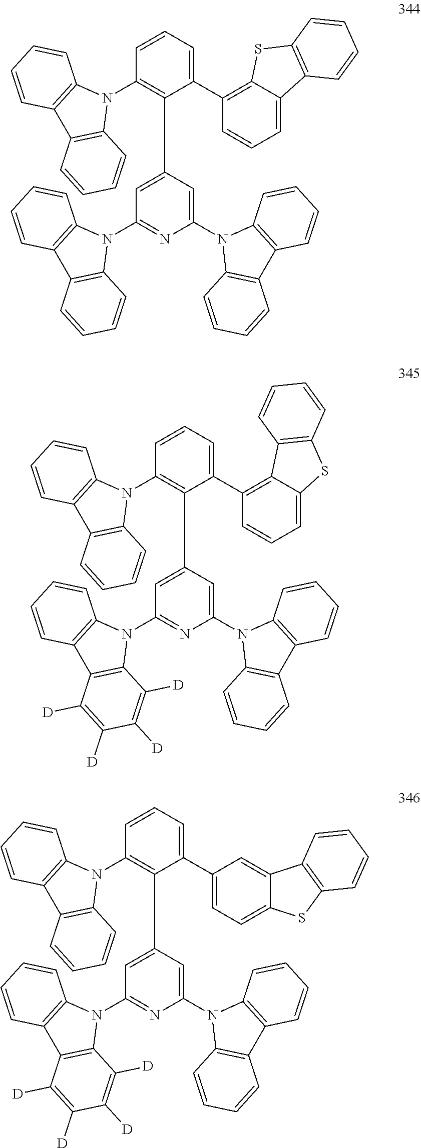 [SMILES: c1cc(-c2cccc3c2sc2ccccc23)c(-c2cc(-n3c4ccccc4c4ccccc43)nc(-n3c4ccccc4c4ccccc43)c2)c(-n2c3ccccc3c3ccccc32)c1 ; [2H]c1c([2H])c([2H])c2c(c1[2H])c1ccccc1n2-c1cc(-c2c(-c3cccc4sc5ccccc5c34)cccc2-n2c3ccccc3c3ccccc32)cc(-n2c3ccccc3c3ccccc32)n1 ; [2H]c1c([2H])c([2H])c2c(c1[2H])c1ccccc1n2-c1cc(-c2c(-c3ccc4sc5ccccc5c4c3)cccc2-n2c3ccccc3c3ccccc32)cc(-n2c3ccccc3c3ccccc32)n1] 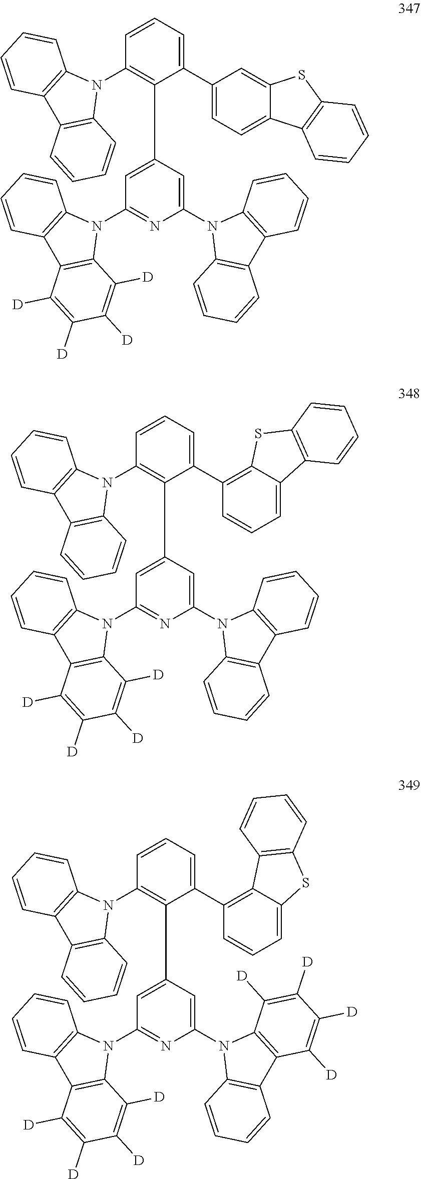 [SMILES: [2H]c1c([2H])c([2H])c2c(c1[2H])c1ccccc1n2-c1cc(-c2c(-c3ccc4c(c3)sc3ccccc34)cccc2-n2c3ccccc3c3ccccc32)cc(-n2c3ccccc3c3ccccc32)n1 ; [2H]c1c([2H])c([2H])c2c(c1[2H])c1ccccc1n2-c1cc(-c2c(-c3cccc4c3sc3ccccc34)cccc2-n2c3ccccc3c3ccccc32)cc(-n2c3ccccc3c3ccccc32)n1 ; [2H]c1c([2H])c([2H])c2c(c1[2H])c1ccccc1n2-c1cc(-c2c(-c3cccc4sc5ccccc5c34)cccc2-n2c3ccccc3c3ccccc32)cc(-n2c3ccccc3c3c([2H])c([2H])c([2H])c([2H])c32)n1] 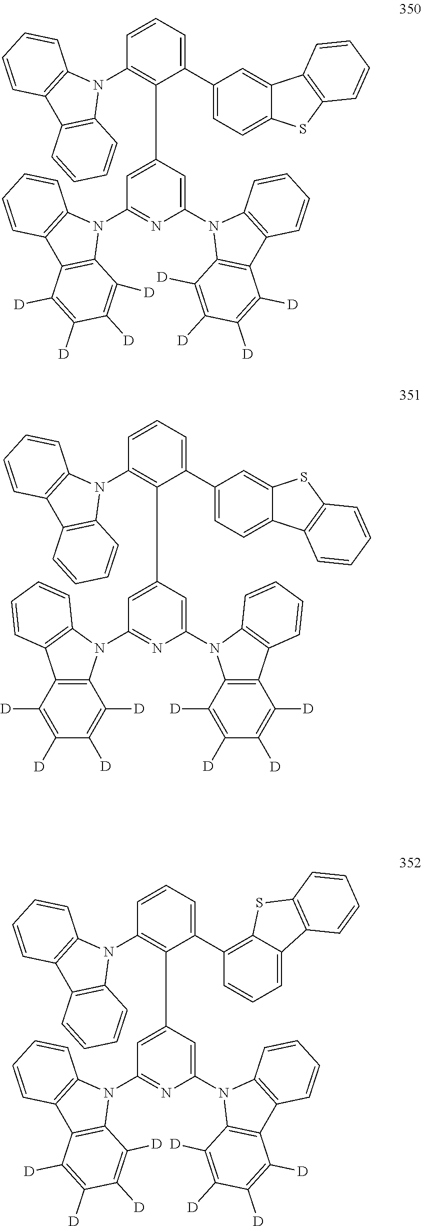 [SMILES: [2H]c1c([2H])c([2H])c2c(c1[2H])c1ccccc1n2-c1cc(-c2c(-c3ccc4sc5ccccc5c4c3)cccc2-n2c3ccccc3c3ccccc32)cc(-n2c3ccccc3c3c([2H])c([2H])c([2H])c([2H])c32)n1 ; [2H]c1c([2H])c([2H])c2c(c1[2H])c1ccccc1n2-c1cc(-c2c(-c3ccc4c(c3)sc3ccccc34)cccc2-n2c3ccccc3c3ccccc32)cc(-n2c3ccccc3c3c([2H])c([2H])c([2H])c([2H])c32)n1 ; [2H]c1c([2H])c([2H])c2c(c1[2H])c1ccccc1n2-c1cc(-c2c(-c3cccc4c3sc3ccccc34)cccc2-n2c3ccccc3c3ccccc32)cc(-n2c3ccccc3c3c([2H])c([2H])c([2H])c([2H])c32)n1] 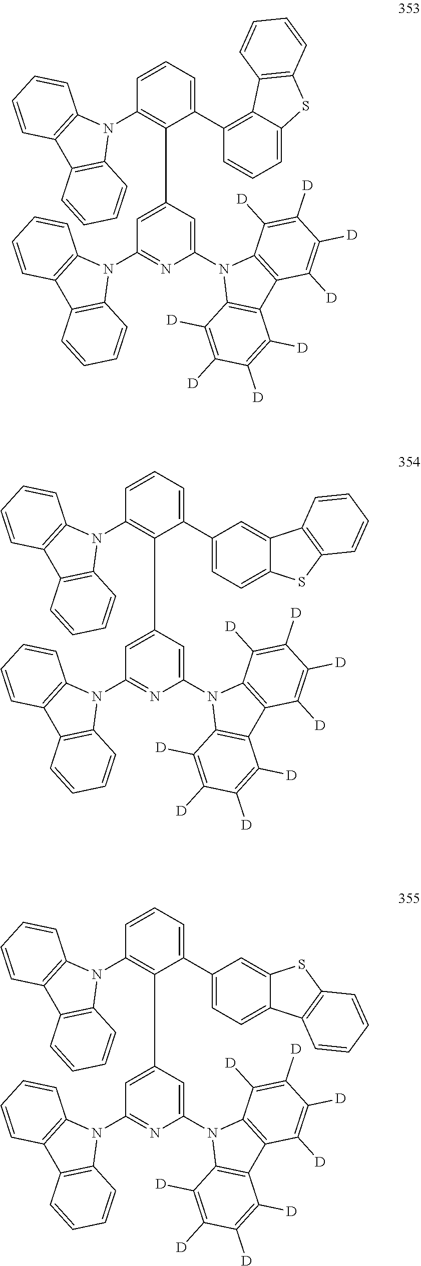 [SMILES: [2H]c1c([2H])c([2H])c2c(c1[2H])c1c([2H])c([2H])c([2H])c([2H])c1n2-c1cc(-c2c(-c3cccc4sc5ccccc5c34)cccc2-n2c3ccccc3c3ccccc32)cc(-n2c3ccccc3c3ccccc32)n1 ; [2H]c1c([2H])c([2H])c2c(c1[2H])c1c([2H])c([2H])c([2H])c([2H])c1n2-c1cc(-c2c(-c3ccc4sc5ccccc5c4c3)cccc2-n2c3ccccc3c3ccccc32)cc(-n2c3ccccc3c3ccccc32)n1 ; [2H]c1c([2H])c([2H])c2c(c1[2H])c1c([2H])c([2H])c([2H])c([2H])c1n2-c1cc(-c2c(-c3ccc4c(c3)sc3ccccc34)cccc2-n2c3ccccc3c3ccccc32)cc(-n2c3ccccc3c3ccccc32)n1] 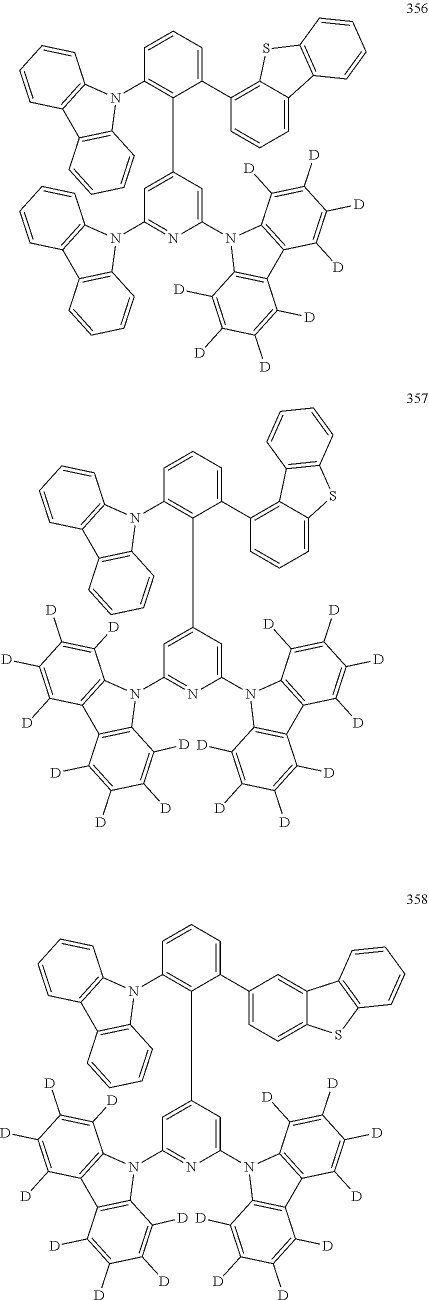 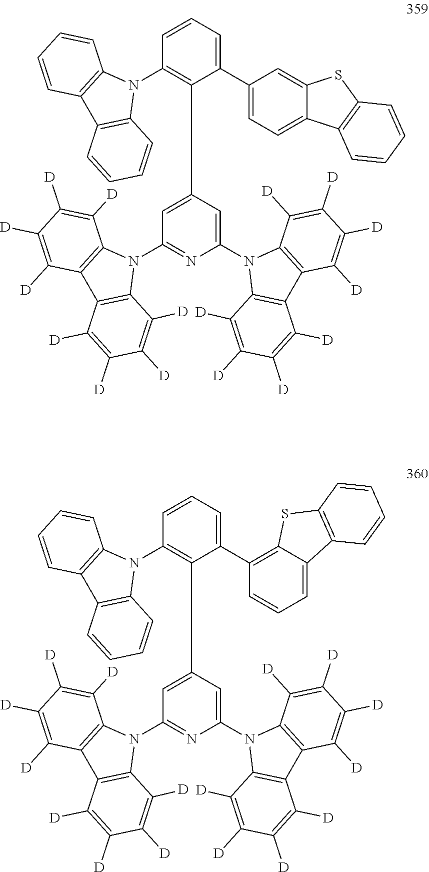 [SMILES: [2H]c1c([2H])c([2H])c2c(c1[2H])c1c([2H])c([2H])c([2H])c([2H])c1n2-c1cc(-c2c(-c3ccc4c(c3)sc3ccccc34)cccc2-n2c3ccccc3c3ccccc32)cc(-n2c3c([2H])c([2H])c([2H])c([2H])c3c3c([2H])c([2H])c([2H])c([2H])c32)n1 ; [2H]c1c([2H])c([2H])c2c(c1[2H])c1c([2H])c([2H])c([2H])c([2H])c1n2-c1cc(-c2c(-c3cccc4c3sc3ccccc34)cccc2-n2c3ccccc3c3ccccc32)cc(-n2c3c([2H])c([2H])c([2H])c([2H])c3c3c([2H])c([2H])c([2H])c([2H])c32)n1] 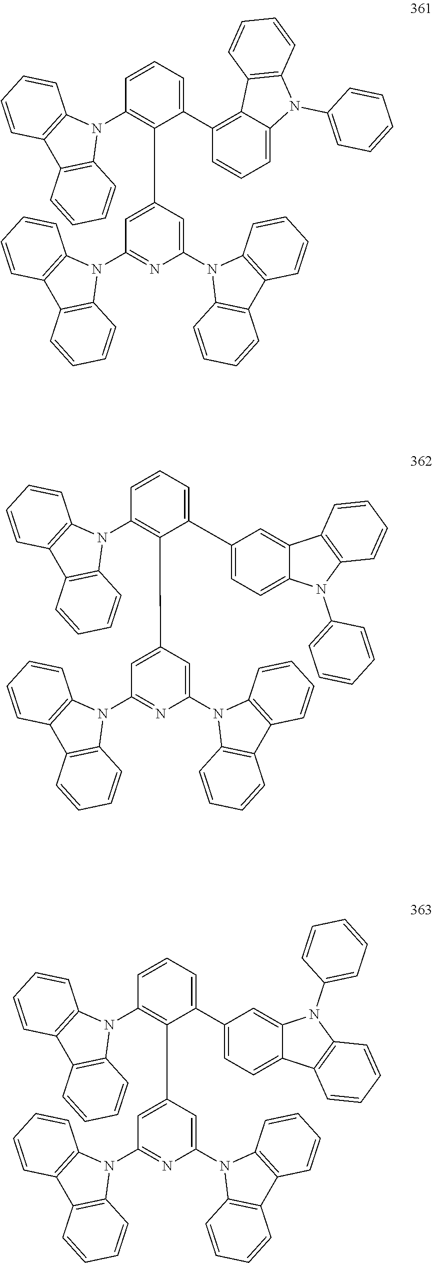 [SMILES: c1ccc(-n2c3ccccc3c3c(-c4cccc(-n5c6ccccc6c6ccccc65)c4-c4cc(-n5c6ccccc6c6ccccc65)nc(-n5c6ccccc6c6ccccc65)c4)cccc32)cc1 ; c1ccc(-n2c3ccccc3c3cc(-c4cccc(-n5c6ccccc6c6ccccc65)c4-c4cc(-n5c6ccccc6c6ccccc65)nc(-n5c6ccccc6c6ccccc65)c4)ccc32)cc1 ; c1ccc(-n2c3ccccc3c3ccc(-c4cccc(-n5c6ccccc6c6ccccc65)c4-c4cc(-n5c6ccccc6c6ccccc65)nc(-n5c6ccccc6c6ccccc65)c4)cc32)cc1] 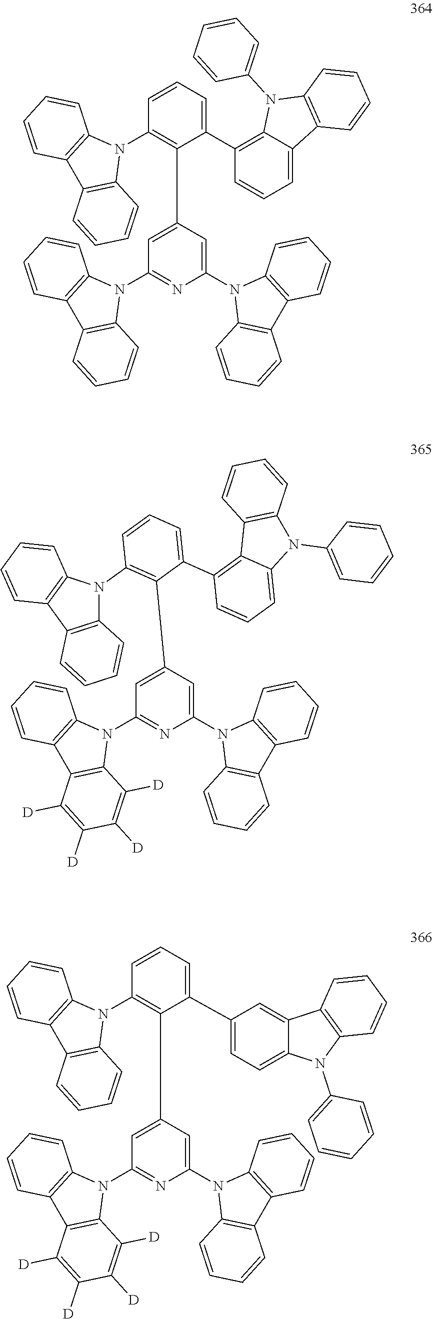 [SMILES: c1ccc(-n2c3ccccc3c3cccc(-c4cccc(-n5c6ccccc6c6ccccc65)c4-c4cc(-n5c6ccccc6c6ccccc65)nc(-n5c6ccccc6c6ccccc65)c4)c32)cc1 ; [2H]c1c([2H])c([2H])c2c(c1[2H])c1ccccc1n2-c1cc(-c2c(-c3cccc4c3c3ccccc3n4-c3ccccc3)cccc2-n2c3ccccc3c3ccccc32)cc(-n2c3ccccc3c3ccccc32)n1 ; [2H]c1c([2H])c([2H])c2c(c1[2H])c1ccccc1n2-c1cc(-c2c(-c3ccc4c(c3)c3ccccc3n4-c3ccccc3)cccc2-n2c3ccccc3c3ccccc32)cc(-n2c3ccccc3c3ccccc32)n1] 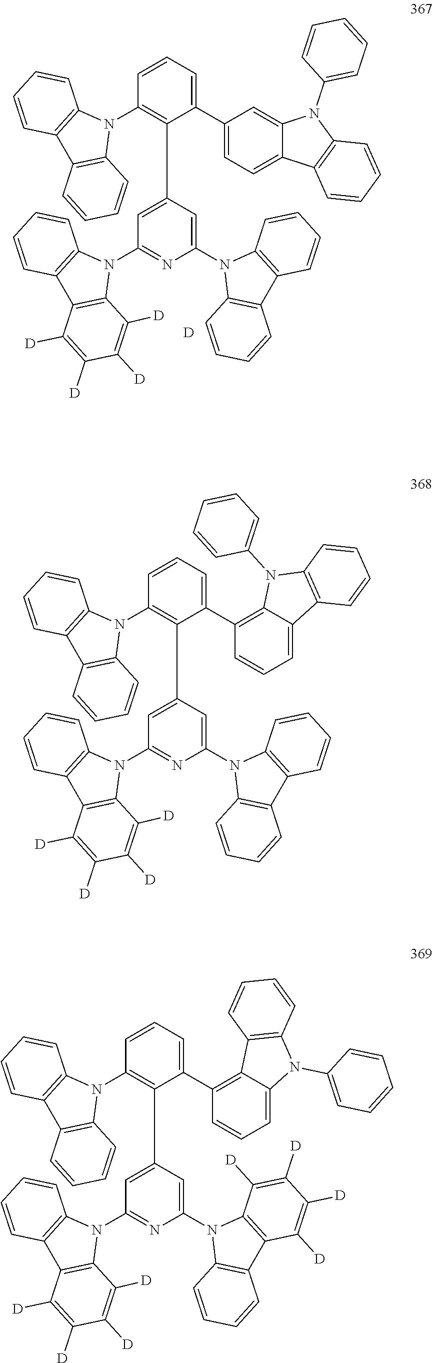 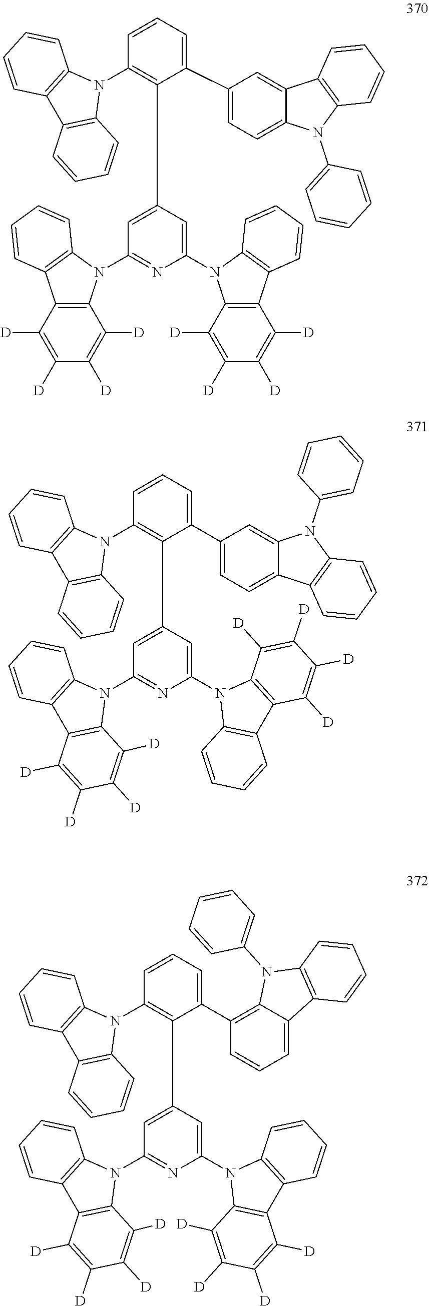 [SMILES: [2H]c1c([2H])c([2H])c2c(c1[2H])c1ccccc1n2-c1cc(-c2c(-c3ccc4c(c3)c3ccccc3n4-c3ccccc3)cccc2-n2c3ccccc3c3ccccc32)cc(-n2c3ccccc3c3c([2H])c([2H])c([2H])c([2H])c32)n1 ; [2H]c1c([2H])c([2H])c2c(c1[2H])c1ccccc1n2-c1cc(-c2c(-c3ccc4c5ccccc5n(-c5ccccc5)c4c3)cccc2-n2c3ccccc3c3ccccc32)cc(-n2c3ccccc3c3c([2H])c([2H])c([2H])c([2H])c32)n1 ; [2H]c1c([2H])c([2H])c2c(c1[2H])c1ccccc1n2-c1cc(-c2c(-c3cccc4c5ccccc5n(-c5ccccc5)c34)cccc2-n2c3ccccc3c3ccccc32)cc(-n2c3ccccc3c3c([2H])c([2H])c([2H])c([2H])c32)n1] 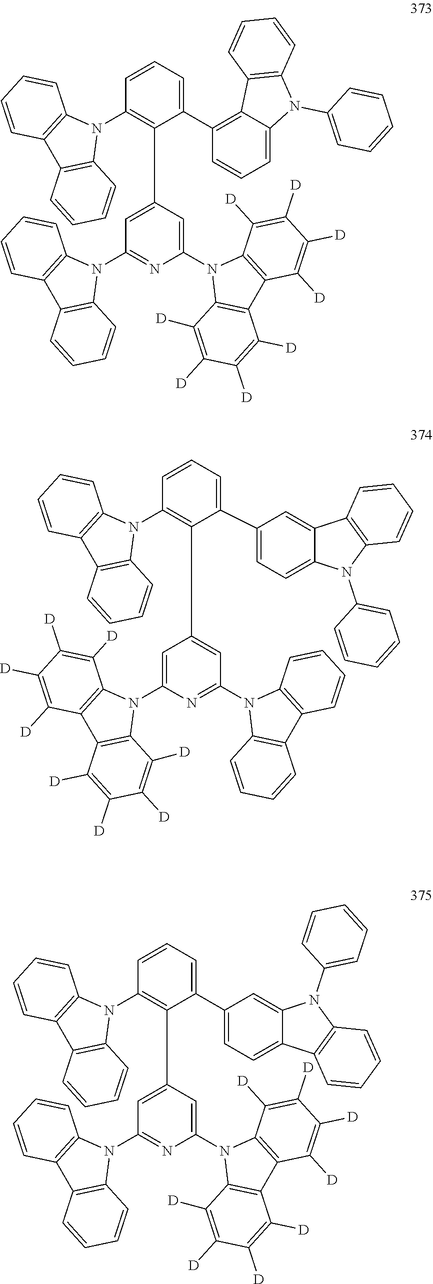 [SMILES: [2H]c1c([2H])c([2H])c2c(c1[2H])c1c([2H])c([2H])c([2H])c([2H])c1n2-c1cc(-c2c(-c3cccc4c3c3ccccc3n4-c3ccccc3)cccc2-n2c3ccccc3c3ccccc32)cc(-n2c3ccccc3c3ccccc32)n1 ; [2H]c1c([2H])c([2H])c2c(c1[2H])c1c([2H])c([2H])c([2H])c([2H])c1n2-c1cc(-c2c(-c3ccc4c(c3)c3ccccc3n4-c3ccccc3)cccc2-n2c3ccccc3c3ccccc32)cc(-n2c3ccccc3c3ccccc32)n1 ; [2H]c1c([2H])c([2H])c2c(c1[2H])c1c([2H])c([2H])c([2H])c([2H])c1n2-c1cc(-c2c(-c3ccc4c5ccccc5n(-c5ccccc5)c4c3)cccc2-n2c3ccccc3c3ccccc32)cc(-n2c3ccccc3c3ccccc32)n1] 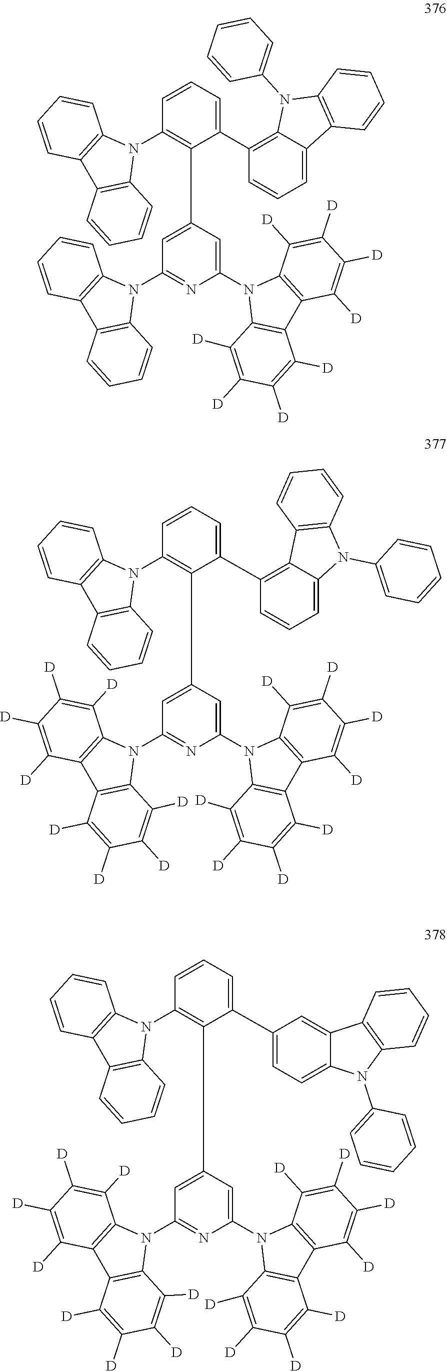 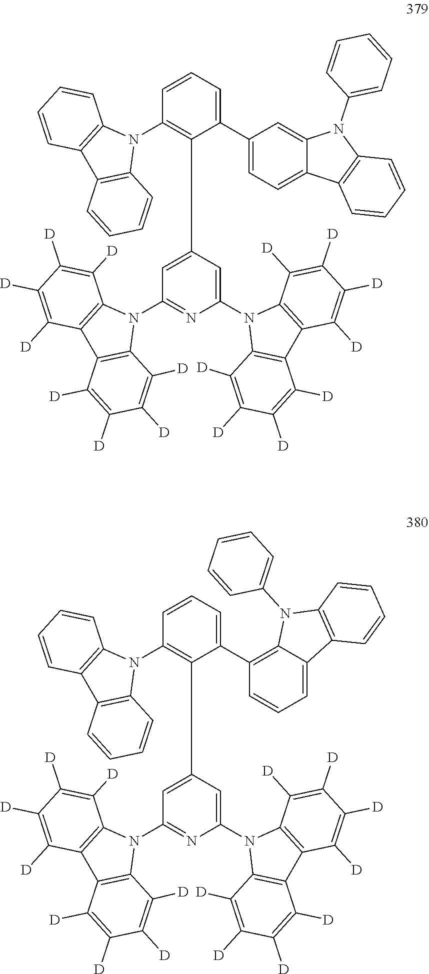 [SMILES: [2H]c1c([2H])c([2H])c2c(c1[2H])c1c([2H])c([2H])c([2H])c([2H])c1n2-c1cc(-c2c(-c3ccc4c5ccccc5n(-c5ccccc5)c4c3)cccc2-n2c3ccccc3c3ccccc32)cc(-n2c3c([2H])c([2H])c([2H])c([2H])c3c3c([2H])c([2H])c([2H])c([2H])c32)n1 ; [2H]c1c([2H])c([2H])c2c(c1[2H])c1c([2H])c([2H])c([2H])c([2H])c1n2-c1cc(-c2c(-c3cccc4c5ccccc5n(-c5ccccc5)c34)cccc2-n2c3ccccc3c3ccccc32)cc(-n2c3c([2H])c([2H])c([2H])c([2H])c3c3c([2H])c([2H])c([2H])c([2H])c32)n1] 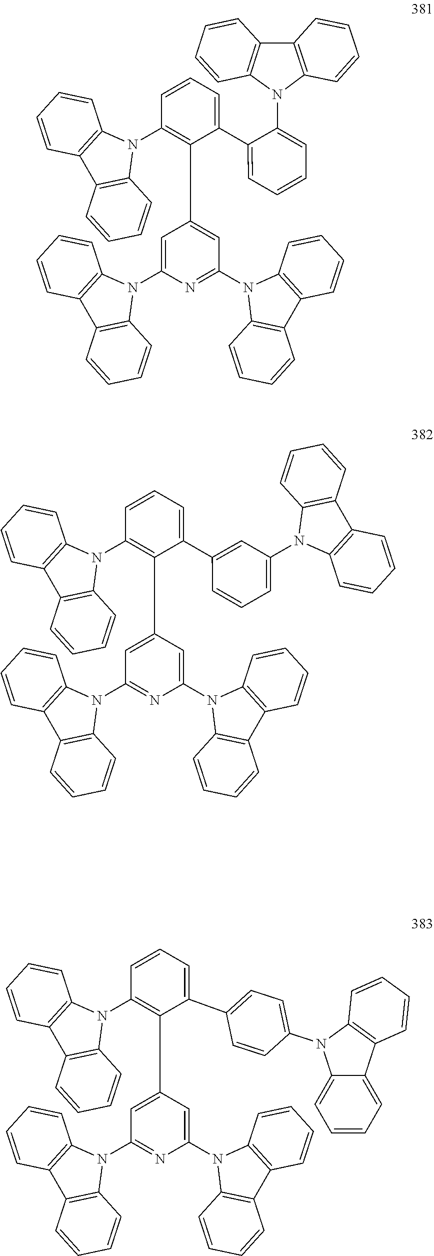 [SMILES: c1ccc(-n2c3ccccc3c3ccccc32)c(-c2cccc(-n3c4ccccc4c4ccccc43)c2-c2cc(-n3c4ccccc4c4ccccc43)nc(-n3c4ccccc4c4ccccc43)c2)c1 ; c1cc(-c2cccc(-n3c4ccccc4c4ccccc43)c2-c2cc(-n3c4ccccc4c4ccccc43)nc(-n3c4ccccc4c4ccccc43)c2)cc(-n2c3ccccc3c3ccccc32)c1 ; c1cc(-c2ccc(-n3c4ccccc4c4ccccc43)cc2)c(-c2cc(-n3c4ccccc4c4ccccc43)nc(-n3c4ccccc4c4ccccc43)c2)c(-n2c3ccccc3c3ccccc32)c1] 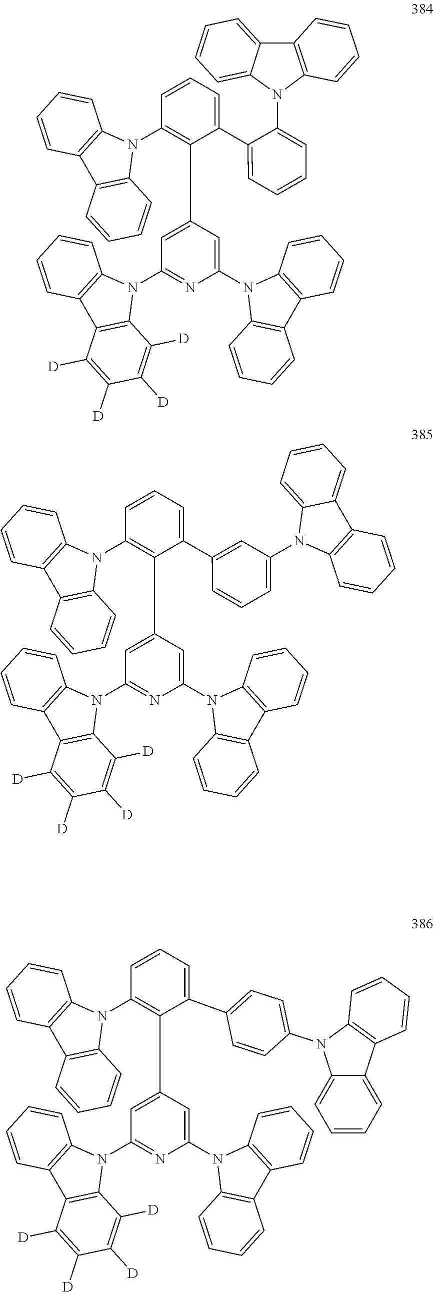 [SMILES: [2H]c1c([2H])c([2H])c2c(c1[2H])c1ccccc1n2-c1cc(-c2c(-c3ccccc3-n3c4ccccc4c4ccccc43)cccc2-n2c3ccccc3c3ccccc32)cc(-n2c3ccccc3c3ccccc32)n1 ; [2H]c1c([2H])c([2H])c2c(c1[2H])c1ccccc1n2-c1cc(-c2c(-c3cccc(-n4c5ccccc5c5ccccc54)c3)cccc2-n2c3ccccc3c3ccccc32)cc(-n2c3ccccc3c3ccccc32)n1 ; [2H]c1c([2H])c([2H])c2c(c1[2H])c1ccccc1n2-c1cc(-c2c(-c3ccc(-n4c5ccccc5c5ccccc54)cc3)cccc2-n2c3ccccc3c3ccccc32)cc(-n2c3ccccc3c3ccccc32)n1] 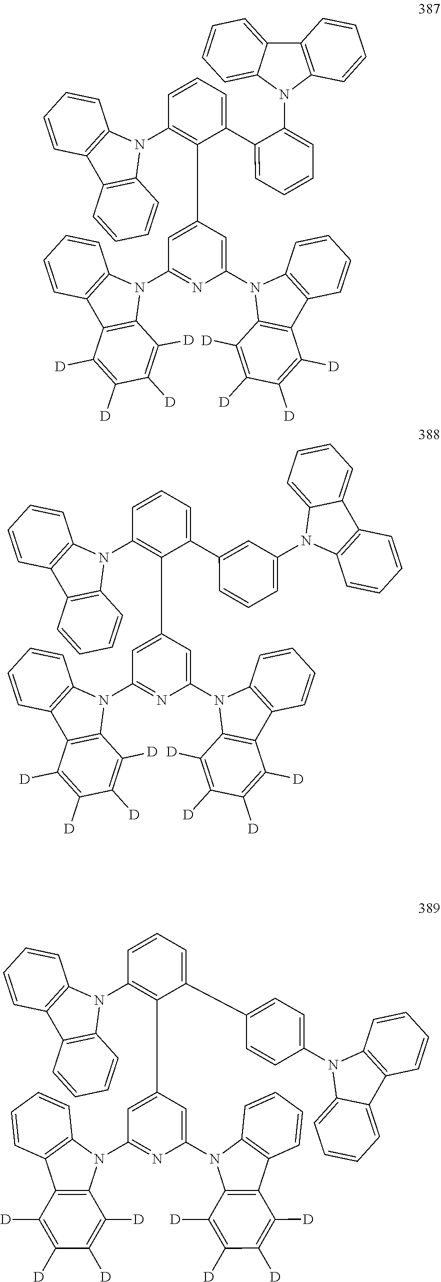 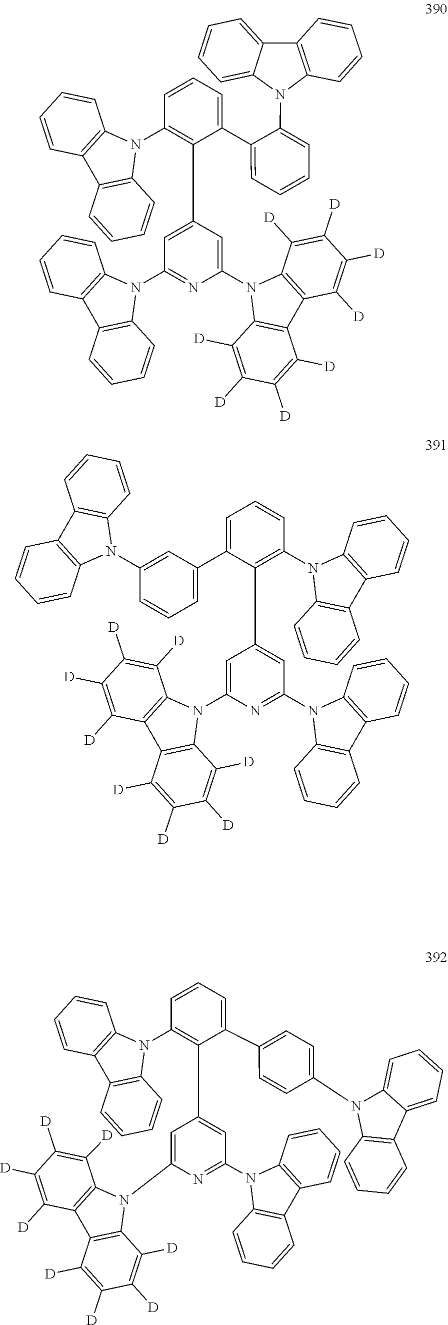 [SMILES: [2H]c1c([2H])c([2H])c2c(c1[2H])c1c([2H])c([2H])c([2H])c([2H])c1n2-c1cc(-c2c(-c3ccccc3-n3c4ccccc4c4ccccc43)cccc2-n2c3ccccc3c3ccccc32)cc(-n2c3ccccc3c3ccccc32)n1 ; [2H]c1c([2H])c([2H])c2c(c1[2H])c1c([2H])c([2H])c([2H])c([2H])c1n2-c1cc(-c2c(-c3cccc(-n4c5ccccc5c5ccccc54)c3)cccc2-n2c3ccccc3c3ccccc32)cc(-n2c3ccccc3c3ccccc32)n1 ; [2H]c1c([2H])c([2H])c2c(c1[2H])c1c([2H])c([2H])c([2H])c([2H])c1n2-c1cc(-c2c(-c3ccc(-n4c5ccccc5c5ccccc54)cc3)cccc2-n2c3ccccc3c3ccccc32)cc(-n2c3ccccc3c3ccccc32)n1] 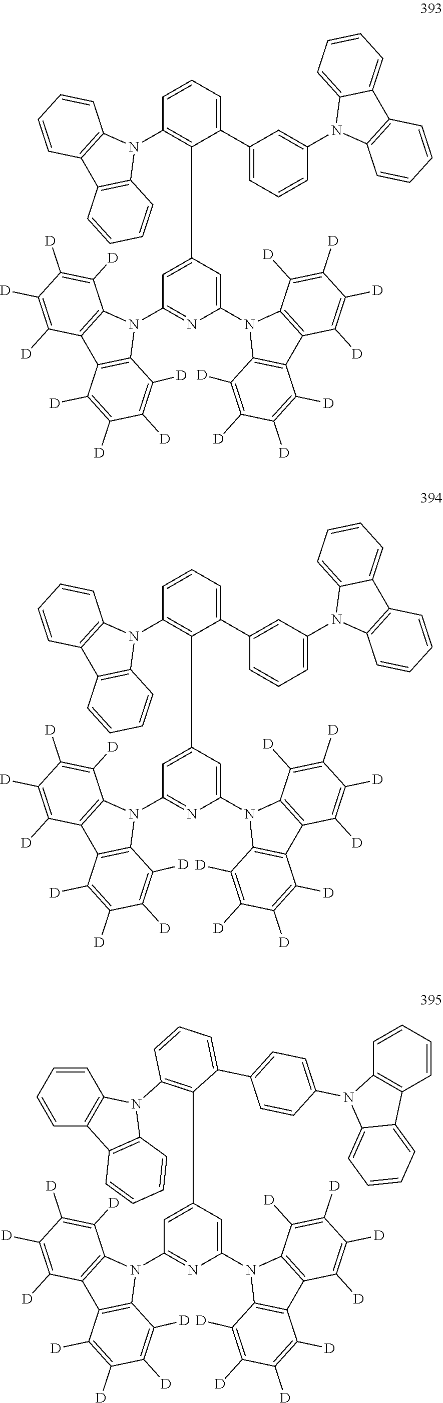 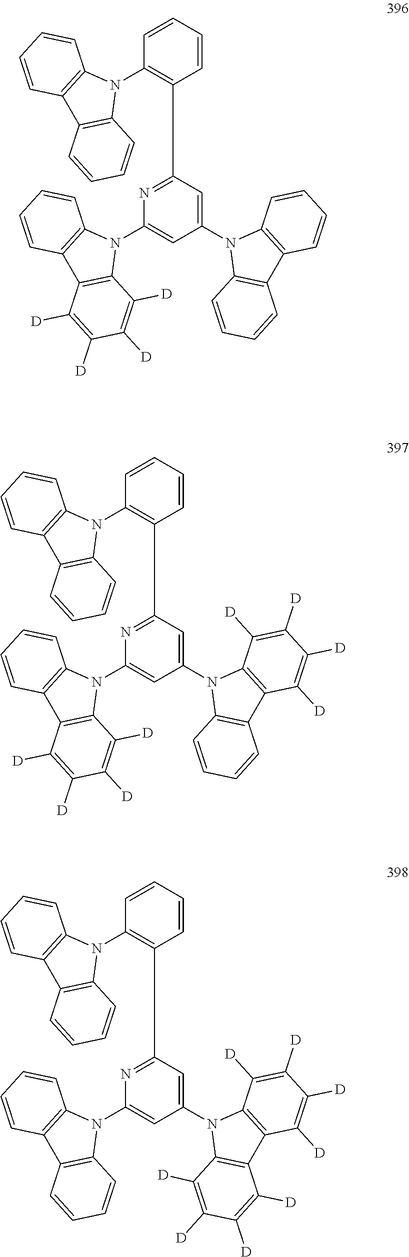 [SMILES: [2H]c1c([2H])c([2H])c2c(c1[2H])c1ccccc1n2-c1cc(-n2c3ccccc3c3ccccc32)cc(-c2ccccc2-n2c3ccccc3c3ccccc32)n1 ; [2H]c1c([2H])c([2H])c2c(c1[2H])c1ccccc1n2-c1cc(-c2ccccc2-n2c3ccccc3c3ccccc32)nc(-n2c3ccccc3c3c([2H])c([2H])c([2H])c([2H])c32)c1 ; [2H]c1c([2H])c([2H])c2c(c1[2H])c1c([2H])c([2H])c([2H])c([2H])c1n2-c1cc(-c2ccccc2-n2c3ccccc3c3ccccc32)nc(-n2c3ccccc3c3ccccc32)c1] 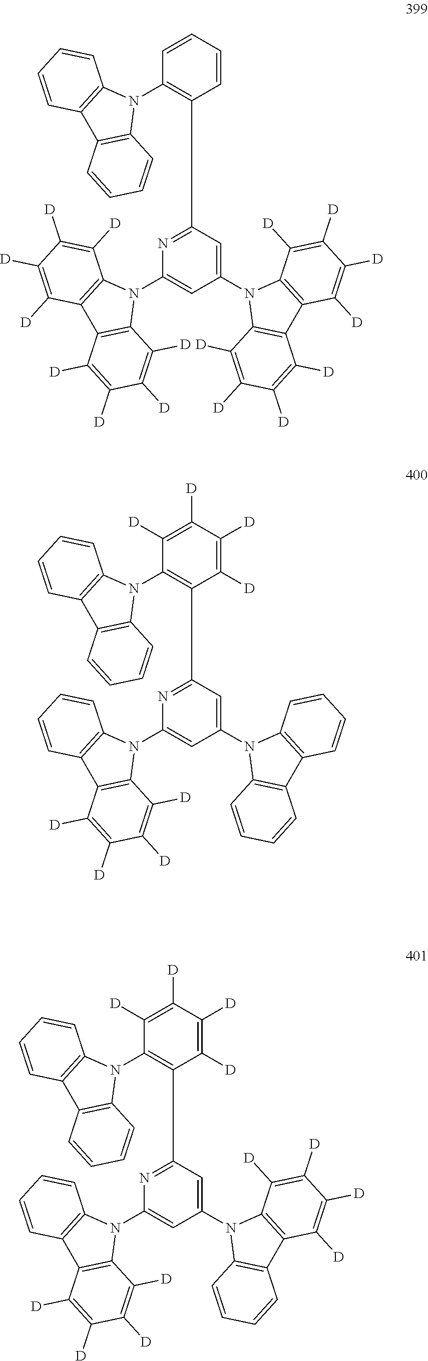 [SMILES: [2H]c1c([2H])c([2H])c2c(c1[2H])c1c([2H])c([2H])c([2H])c([2H])c1n2-c1cc(-c2ccccc2-n2c3ccccc3c3ccccc32)nc(-n2c3c([2H])c([2H])c([2H])c([2H])c3c3c([2H])c([2H])c([2H])c([2H])c32)c1 ; [2H]c1c([2H])c([2H])c(-n2c3ccccc3c3ccccc32)c(-c2cc(-n3c4ccccc4c4ccccc43)cc(-n3c4ccccc4c4c([2H])c([2H])c([2H])c([2H])c43)n2)c1[2H] ; [2H]c1c([2H])c([2H])c(-n2c3ccccc3c3ccccc32)c(-c2cc(-n3c4ccccc4c4c([2H])c([2H])c([2H])c([2H])c43)cc(-n3c4ccccc4c4c([2H])c([2H])c([2H])c([2H])c43)n2)c1[2H]] 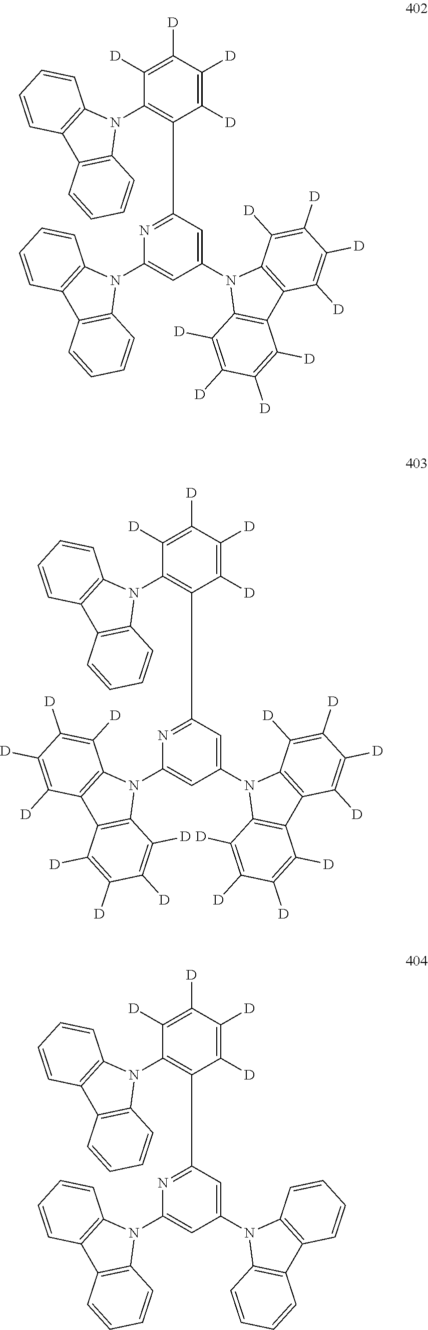 [SMILES: [2H]c1c([2H])c([2H])c(-n2c3ccccc3c3ccccc32)c(-c2cc(-n3c4c([2H])c([2H])c([2H])c([2H])c4c4c([2H])c([2H])c([2H])c([2H])c43)cc(-n3c4ccccc4c4ccccc43)n2)c1[2H] ; [2H]c1c([2H])c([2H])c(-n2c3ccccc3c3ccccc32)c(-c2cc(-n3c4c([2H])c([2H])c([2H])c([2H])c4c4c([2H])c([2H])c([2H])c([2H])c43)cc(-n3c4c([2H])c([2H])c([2H])c([2H])c4c4c([2H])c([2H])c([2H])c([2H])c43)n2)c1[2H] ; [2H]c1c([2H])c([2H])c(-n2c3ccccc3c3ccccc32)c(-c2cc(-n3c4ccccc4c4ccccc43)cc(-n3c4ccccc4c4ccccc43)n2)c1[2H]] 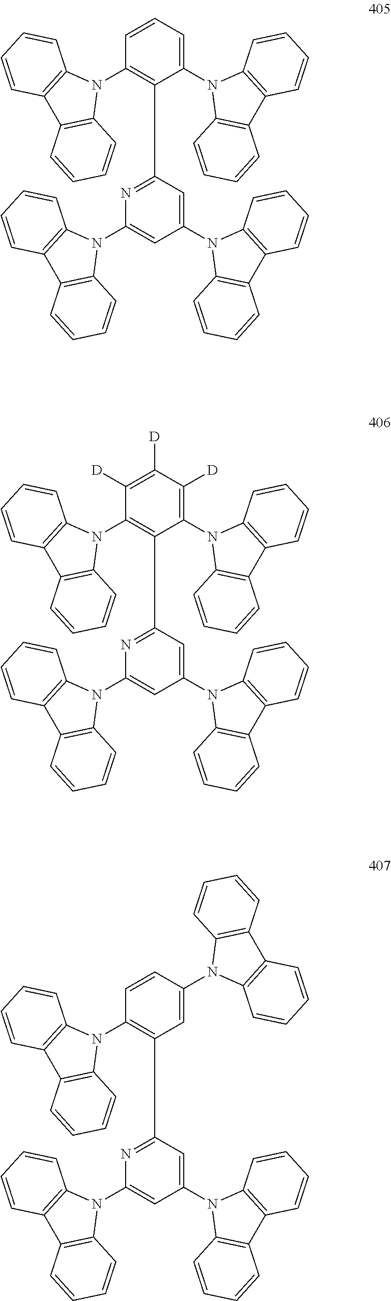 [SMILES: c1cc(-n2c3ccccc3c3ccccc32)c(-c2cc(-n3c4ccccc4c4ccccc43)cc(-n3c4ccccc4c4ccccc43)n2)c(-n2c3ccccc3c3ccccc32)c1 ; [2H]c1c([2H])c(-n2c3ccccc3c3ccccc32)c(-c2cc(-n3c4ccccc4c4ccccc43)cc(-n3c4ccccc4c4ccccc43)n2)c(-n2c3ccccc3c3ccccc32)c1[2H] ; c1ccc2c(c1)c1ccccc1n2-c1cc(-c2cc(-n3c4ccccc4c4ccccc43)ccc2-n2c3ccccc3c3ccccc32)nc(-n2c3ccccc3c3ccccc32)c1] 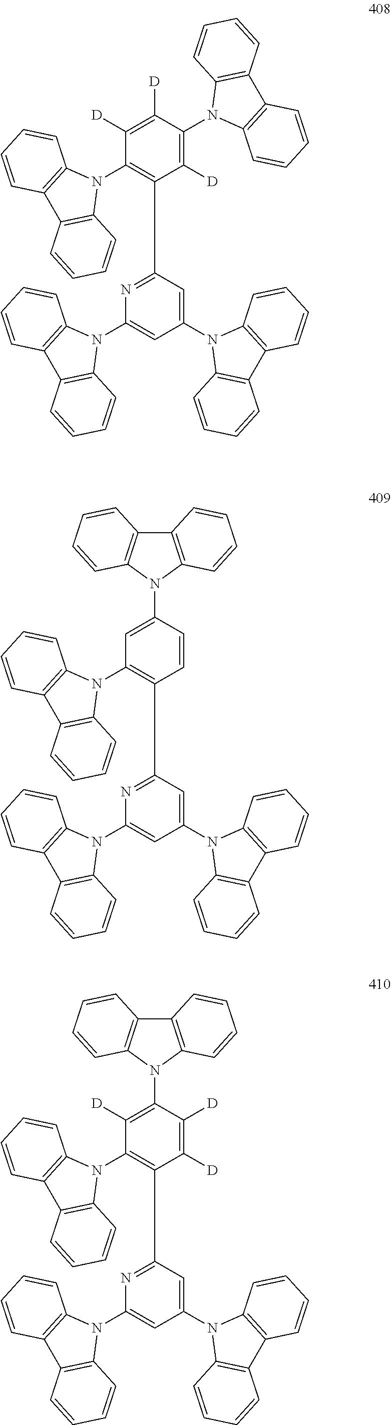 [SMILES: [2H]c1c([2H])c(-n2c3ccccc3c3ccccc32)c(-c2cc(-n3c4ccccc4c4ccccc43)cc(-n3c4ccccc4c4ccccc43)n2)c([2H])c1-n1c2ccccc2c2ccccc21 ; c1ccc2c(c1)c1ccccc1n2-c1cc(-c2ccc(-n3c4ccccc4c4ccccc43)cc2-n2c3ccccc3c3ccccc32)nc(-n2c3ccccc3c3ccccc32)c1 ; [2H]c1c([2H])c(-n2c3ccccc3c3ccccc32)c([2H])c(-n2c3ccccc3c3ccccc32)c1-c1cc(-n2c3ccccc3c3ccccc32)cc(-n2c3ccccc3c3ccccc32)n1] 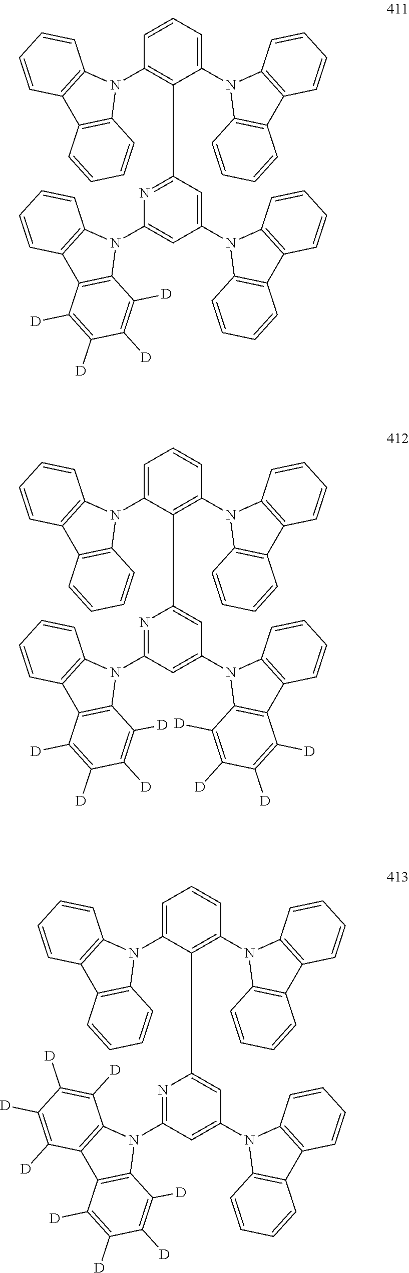 [SMILES: [2H]c1c([2H])c([2H])c2c(c1[2H])c1ccccc1n2-c1cc(-n2c3ccccc3c3ccccc32)cc(-c2c(-n3c4ccccc4c4ccccc43)cccc2-n2c3ccccc3c3ccccc32)n1 ; [2H]c1c([2H])c([2H])c2c(c1[2H])c1ccccc1n2-c1cc(-c2c(-n3c4ccccc4c4ccccc43)cccc2-n2c3ccccc3c3ccccc32)nc(-n2c3ccccc3c3c([2H])c([2H])c([2H])c([2H])c32)c1 ; [2H]c1c([2H])c([2H])c2c(c1[2H])c1c([2H])c([2H])c([2H])c([2H])c1n2-c1cc(-n2c3ccccc3c3ccccc32)cc(-c2c(-n3c4ccccc4c4ccccc43)cccc2-n2c3ccccc3c3ccccc32)n1] 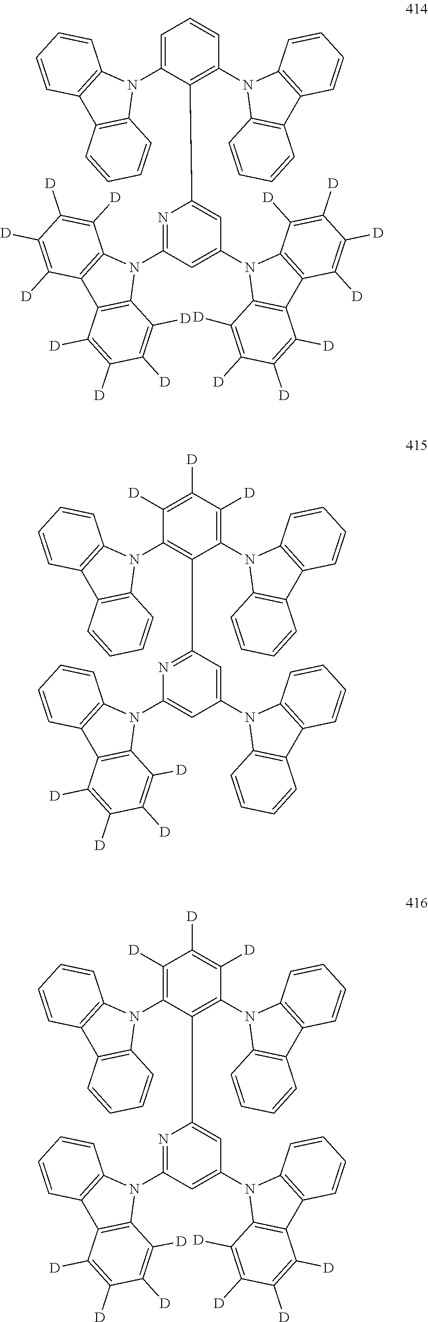 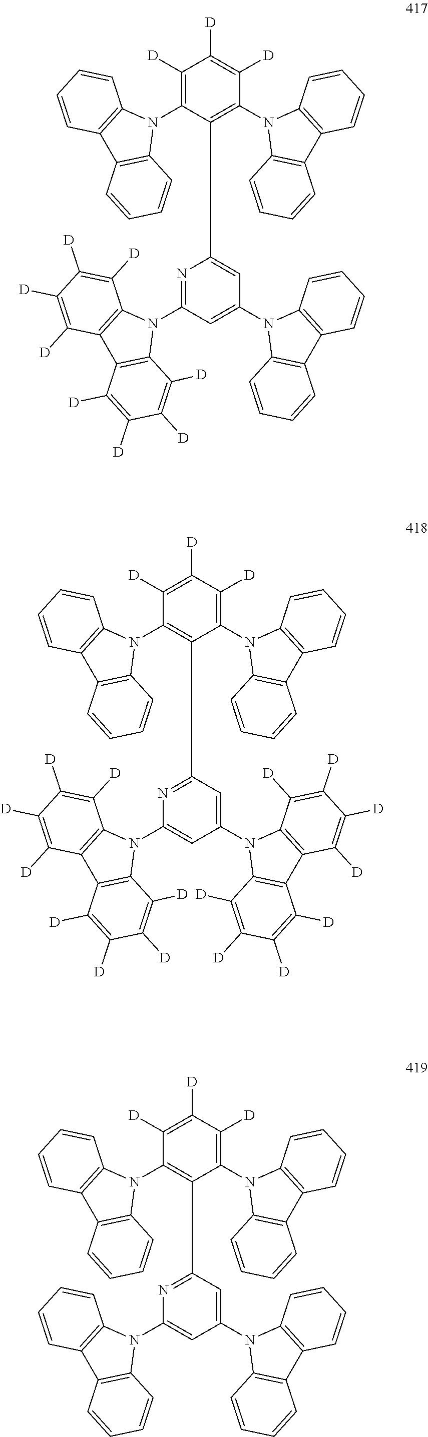 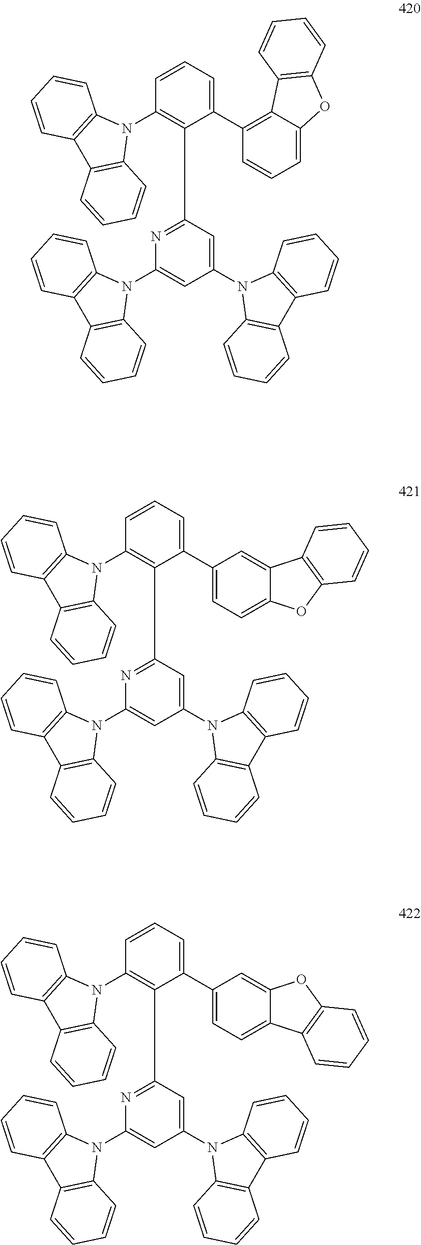 [SMILES: c1cc(-c2cccc3oc4ccccc4c23)c(-c2cc(-n3c4ccccc4c4ccccc43)cc(-n3c4ccccc4c4ccccc43)n2)c(-n2c3ccccc3c3ccccc32)c1 ; c1cc(-c2ccc3oc4ccccc4c3c2)c(-c2cc(-n3c4ccccc4c4ccccc43)cc(-n3c4ccccc4c4ccccc43)n2)c(-n2c3ccccc3c3ccccc32)c1 ; c1cc(-c2ccc3c(c2)oc2ccccc23)c(-c2cc(-n3c4ccccc4c4ccccc43)cc(-n3c4ccccc4c4ccccc43)n2)c(-n2c3ccccc3c3ccccc32)c1] 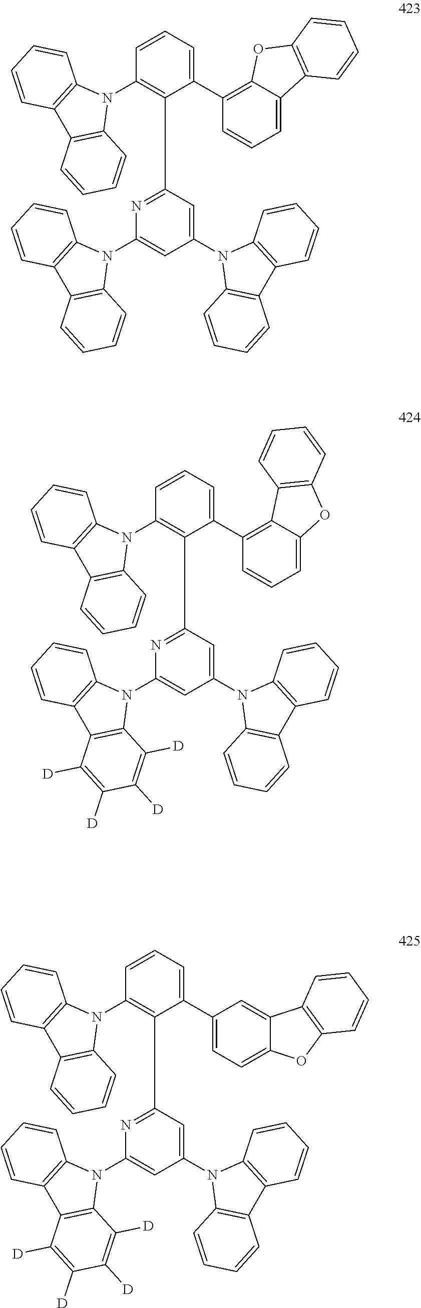 [SMILES: c1cc(-c2cccc3c2oc2ccccc23)c(-c2cc(-n3c4ccccc4c4ccccc43)cc(-n3c4ccccc4c4ccccc43)n2)c(-n2c3ccccc3c3ccccc32)c1 ; [2H]c1c([2H])c([2H])c2c(c1[2H])c1ccccc1n2-c1cc(-n2c3ccccc3c3ccccc32)cc(-c2c(-c3cccc4oc5ccccc5c34)cccc2-n2c3ccccc3c3ccccc32)n1 ; [2H]c1c([2H])c([2H])c2c(c1[2H])c1ccccc1n2-c1cc(-n2c3ccccc3c3ccccc32)cc(-c2c(-c3ccc4oc5ccccc5c4c3)cccc2-n2c3ccccc3c3ccccc32)n1] 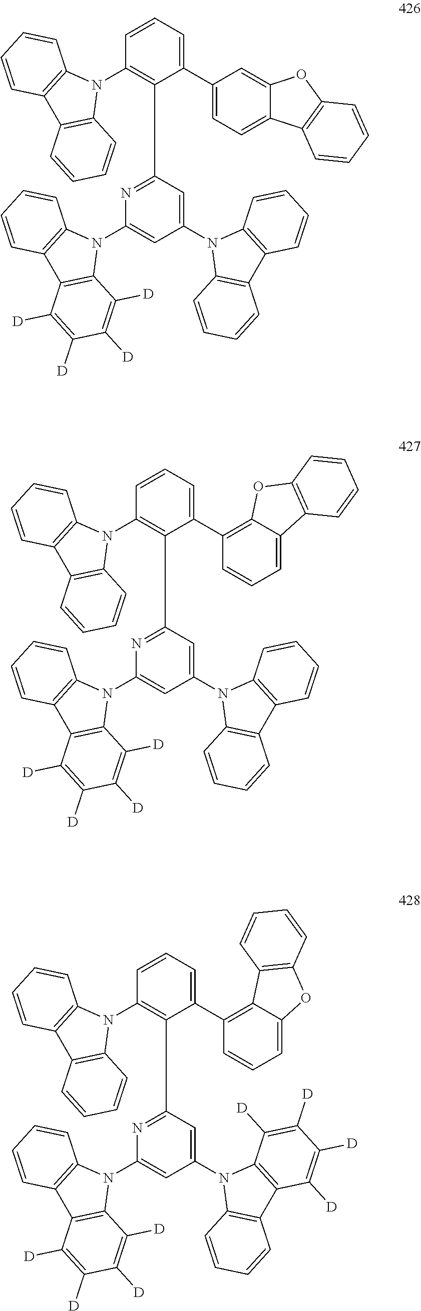 [SMILES: [2H]c1c([2H])c([2H])c2c(c1[2H])c1ccccc1n2-c1cc(-n2c3ccccc3c3ccccc32)cc(-c2c(-c3ccc4c(c3)oc3ccccc34)cccc2-n2c3ccccc3c3ccccc32)n1 ; [2H]c1c([2H])c([2H])c2c(c1[2H])c1ccccc1n2-c1cc(-n2c3ccccc3c3ccccc32)cc(-c2c(-c3cccc4c3oc3ccccc34)cccc2-n2c3ccccc3c3ccccc32)n1 ; [2H]c1c([2H])c([2H])c2c(c1[2H])c1ccccc1n2-c1cc(-c2c(-c3cccc4oc5ccccc5c34)cccc2-n2c3ccccc3c3ccccc32)nc(-n2c3ccccc3c3c([2H])c([2H])c([2H])c([2H])c32)c1] 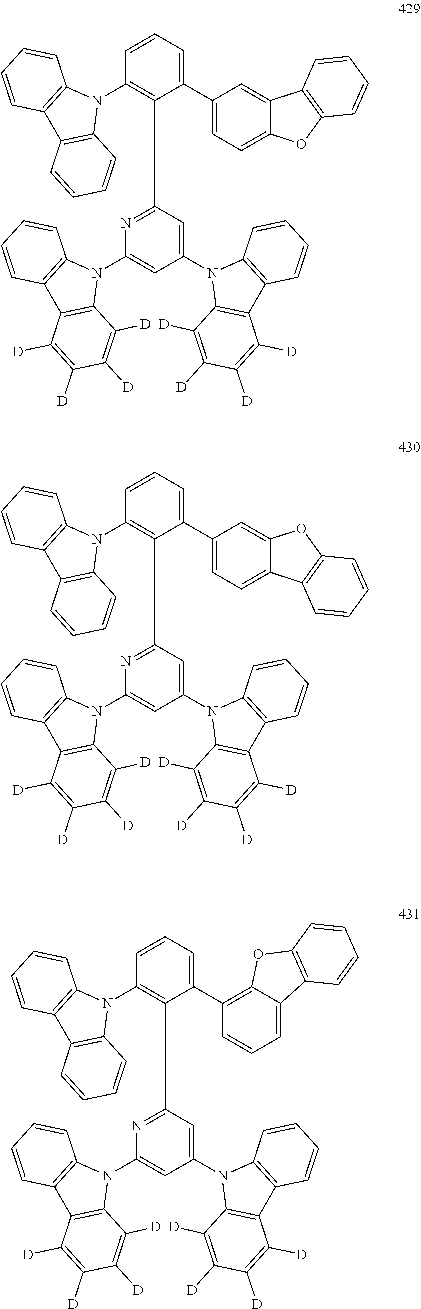 [SMILES: [2H]c1c([2H])c([2H])c2c(c1[2H])c1ccccc1n2-c1cc(-c2c(-c3ccc4oc5ccccc5c4c3)cccc2-n2c3ccccc3c3ccccc32)nc(-n2c3ccccc3c3c([2H])c([2H])c([2H])c([2H])c32)c1 ; [2H]c1c([2H])c([2H])c2c(c1[2H])c1ccccc1n2-c1cc(-c2c(-c3ccc4c(c3)oc3ccccc34)cccc2-n2c3ccccc3c3ccccc32)nc(-n2c3ccccc3c3c([2H])c([2H])c([2H])c([2H])c32)c1 ; [2H]c1c([2H])c([2H])c2c(c1[2H])c1ccccc1n2-c1cc(-c2c(-c3cccc4c3oc3ccccc34)cccc2-n2c3ccccc3c3ccccc32)nc(-n2c3ccccc3c3c([2H])c([2H])c([2H])c([2H])c32)c1] 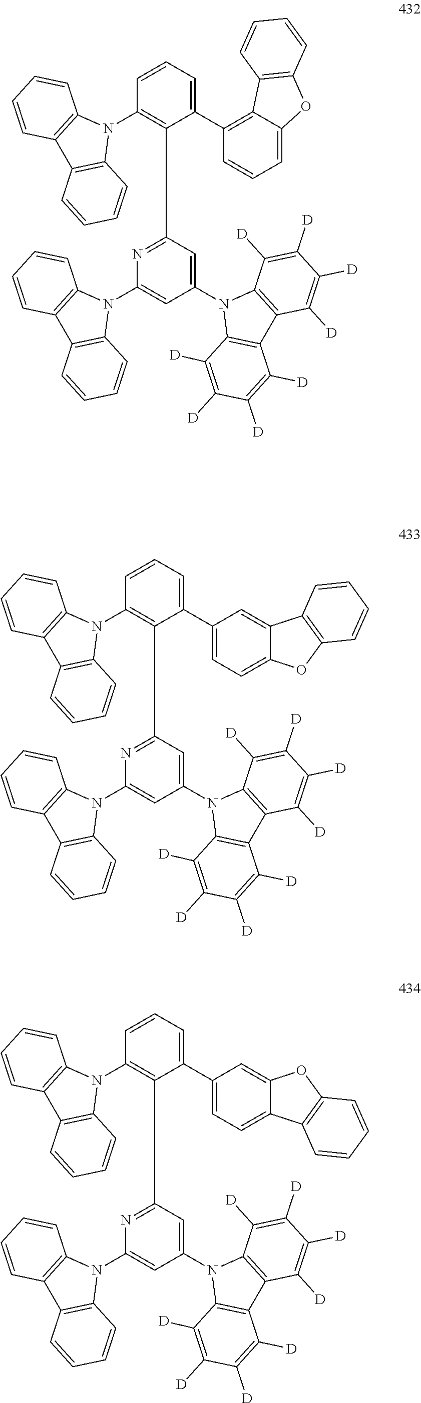 [SMILES: [2H]c1c([2H])c([2H])c2c(c1[2H])c1c([2H])c([2H])c([2H])c([2H])c1n2-c1cc(-c2c(-c3cccc4oc5ccccc5c34)cccc2-n2c3ccccc3c3ccccc32)nc(-n2c3ccccc3c3ccccc32)c1 ; [2H]c1c([2H])c([2H])c2c(c1[2H])c1c([2H])c([2H])c([2H])c([2H])c1n2-c1cc(-c2c(-c3ccc4oc5ccccc5c4c3)cccc2-n2c3ccccc3c3ccccc32)nc(-n2c3ccccc3c3ccccc32)c1 ; [2H]c1c([2H])c([2H])c2c(c1[2H])c1c([2H])c([2H])c([2H])c([2H])c1n2-c1cc(-c2c(-c3ccc4c(c3)oc3ccccc34)cccc2-n2c3ccccc3c3ccccc32)nc(-n2c3ccccc3c3ccccc32)c1] 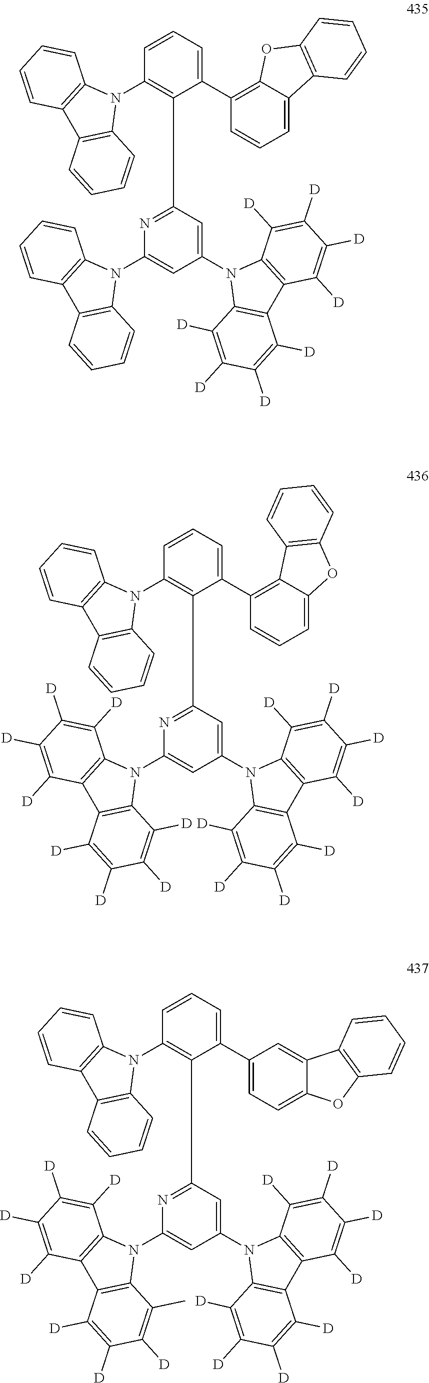 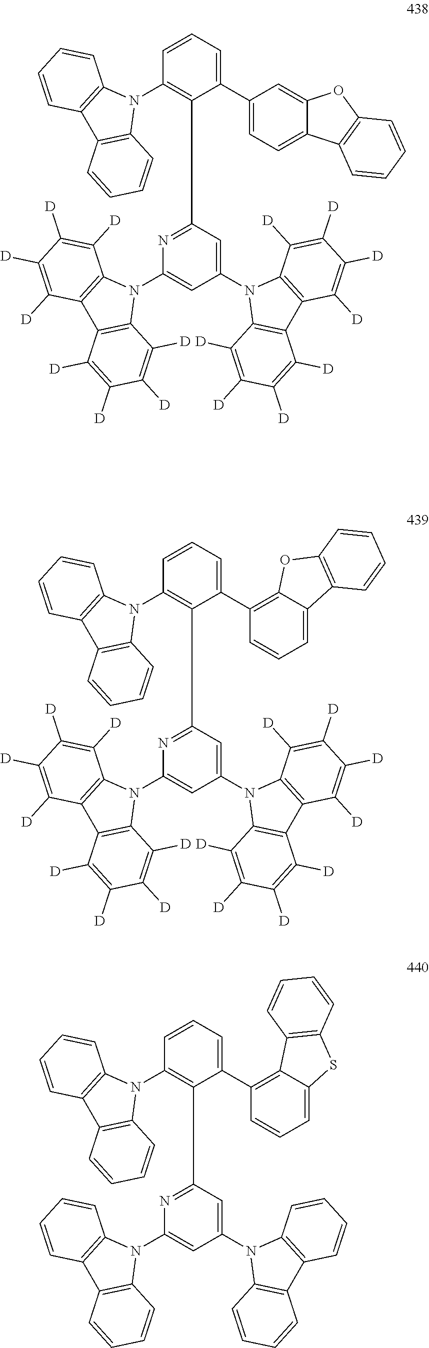 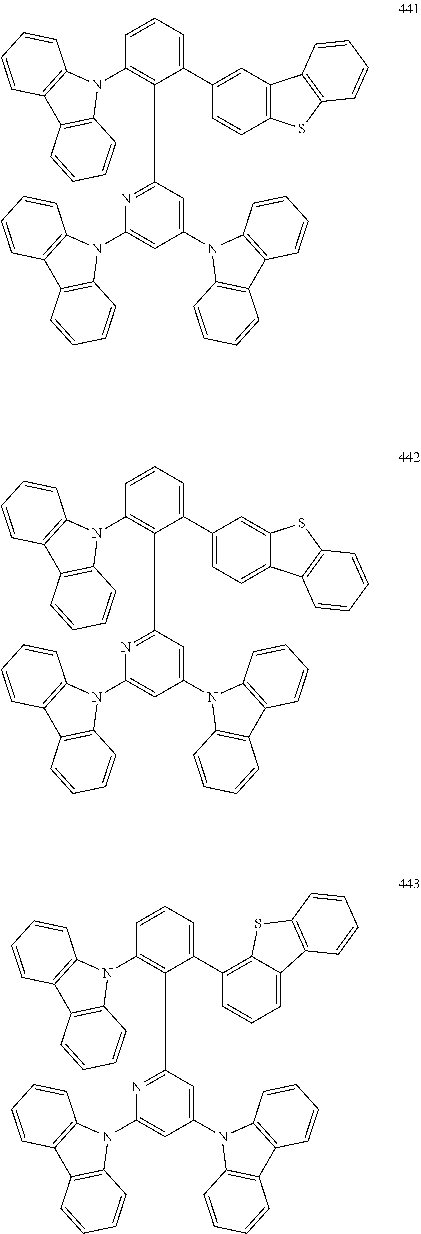 [SMILES: c1cc(-c2ccc3sc4ccccc4c3c2)c(-c2cc(-n3c4ccccc4c4ccccc43)cc(-n3c4ccccc4c4ccccc43)n2)c(-n2c3ccccc3c3ccccc32)c1 ; c1cc(-c2ccc3c(c2)sc2ccccc23)c(-c2cc(-n3c4ccccc4c4ccccc43)cc(-n3c4ccccc4c4ccccc43)n2)c(-n2c3ccccc3c3ccccc32)c1 ; c1cc(-c2cccc3c2sc2ccccc23)c(-c2cc(-n3c4ccccc4c4ccccc43)cc(-n3c4ccccc4c4ccccc43)n2)c(-n2c3ccccc3c3ccccc32)c1] 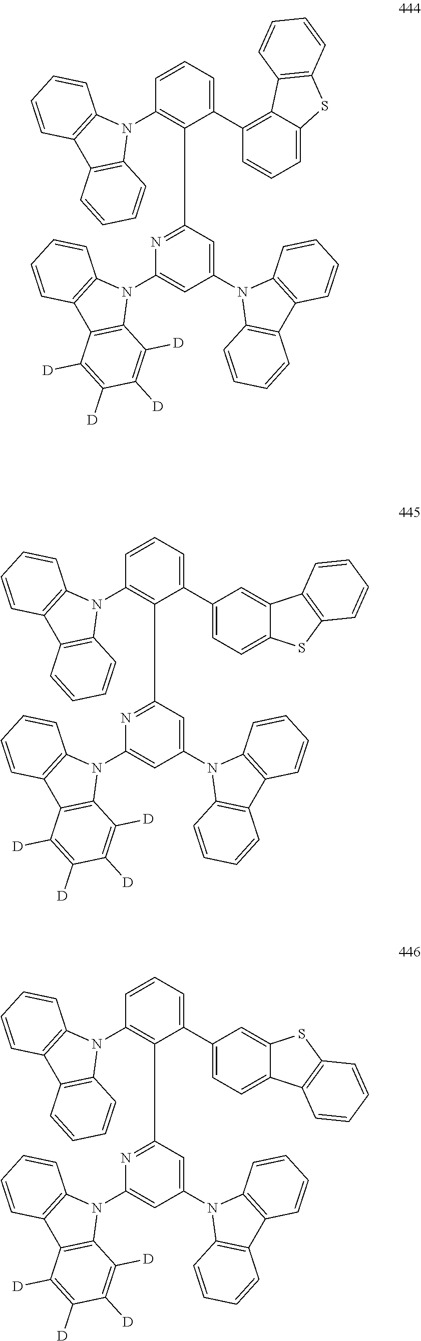 [SMILES: [2H]c1c([2H])c([2H])c2c(c1[2H])c1ccccc1n2-c1cc(-n2c3ccccc3c3ccccc32)cc(-c2c(-c3cccc4sc5ccccc5c34)cccc2-n2c3ccccc3c3ccccc32)n1 ; [2H]c1c([2H])c([2H])c2c(c1[2H])c1ccccc1n2-c1cc(-n2c3ccccc3c3ccccc32)cc(-c2c(-c3ccc4sc5ccccc5c4c3)cccc2-n2c3ccccc3c3ccccc32)n1 ; [2H]c1c([2H])c([2H])c2c(c1[2H])c1ccccc1n2-c1cc(-n2c3ccccc3c3ccccc32)cc(-c2c(-c3ccc4c(c3)sc3ccccc34)cccc2-n2c3ccccc3c3ccccc32)n1] 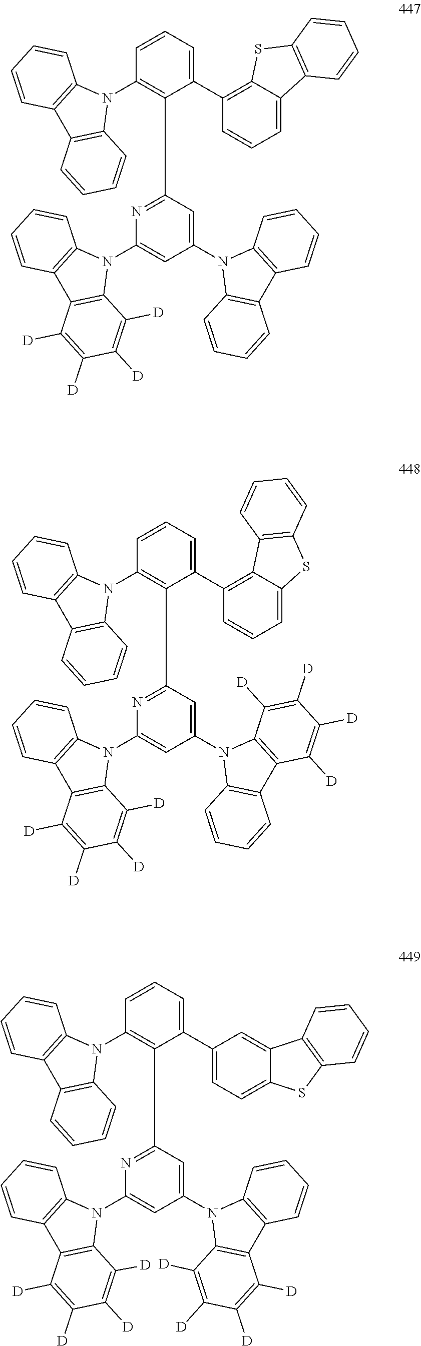 [SMILES: [2H]c1c([2H])c([2H])c2c(c1[2H])c1ccccc1n2-c1cc(-n2c3ccccc3c3ccccc32)cc(-c2c(-c3cccc4c3sc3ccccc34)cccc2-n2c3ccccc3c3ccccc32)n1 ; [2H]c1c([2H])c([2H])c2c(c1[2H])c1ccccc1n2-c1cc(-c2c(-c3cccc4sc5ccccc5c34)cccc2-n2c3ccccc3c3ccccc32)nc(-n2c3ccccc3c3c([2H])c([2H])c([2H])c([2H])c32)c1 ; [2H]c1c([2H])c([2H])c2c(c1[2H])c1ccccc1n2-c1cc(-c2c(-c3ccc4sc5ccccc5c4c3)cccc2-n2c3ccccc3c3ccccc32)nc(-n2c3ccccc3c3c([2H])c([2H])c([2H])c([2H])c32)c1] 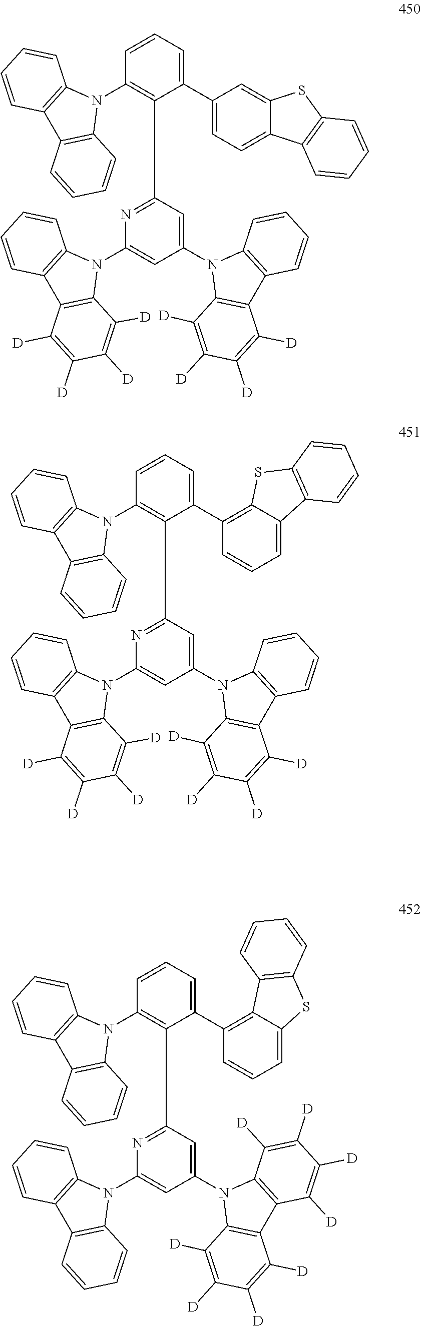 [SMILES: [2H]c1c([2H])c([2H])c2c(c1[2H])c1ccccc1n2-c1cc(-c2c(-c3ccc4c(c3)sc3ccccc34)cccc2-n2c3ccccc3c3ccccc32)nc(-n2c3ccccc3c3c([2H])c([2H])c([2H])c([2H])c32)c1 ; [2H]c1c([2H])c([2H])c2c(c1[2H])c1ccccc1n2-c1cc(-c2c(-c3cccc4c3sc3ccccc34)cccc2-n2c3ccccc3c3ccccc32)nc(-n2c3ccccc3c3c([2H])c([2H])c([2H])c([2H])c32)c1 ; [2H]c1c([2H])c([2H])c2c(c1[2H])c1c([2H])c([2H])c([2H])c([2H])c1n2-c1cc(-c2c(-c3cccc4sc5ccccc5c34)cccc2-n2c3ccccc3c3ccccc32)nc(-n2c3ccccc3c3ccccc32)c1] 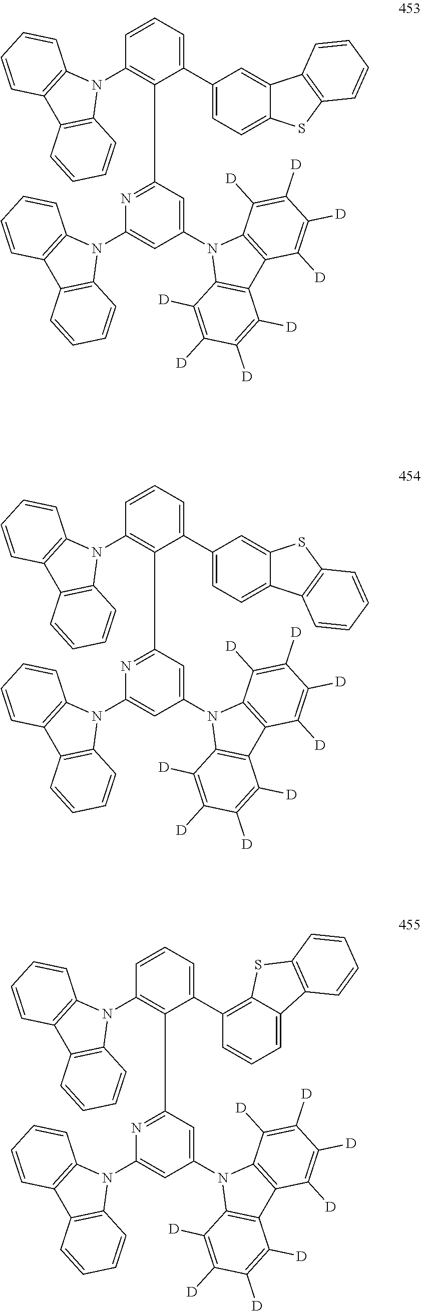 [SMILES: [2H]c1c([2H])c([2H])c2c(c1[2H])c1c([2H])c([2H])c([2H])c([2H])c1n2-c1cc(-c2c(-c3ccc4sc5ccccc5c4c3)cccc2-n2c3ccccc3c3ccccc32)nc(-n2c3ccccc3c3ccccc32)c1 ; [2H]c1c([2H])c([2H])c2c(c1[2H])c1c([2H])c([2H])c([2H])c([2H])c1n2-c1cc(-c2c(-c3ccc4c(c3)sc3ccccc34)cccc2-n2c3ccccc3c3ccccc32)nc(-n2c3ccccc3c3ccccc32)c1 ; [2H]c1c([2H])c([2H])c2c(c1[2H])c1c([2H])c([2H])c([2H])c([2H])c1n2-c1cc(-c2c(-c3cccc4c3sc3ccccc34)cccc2-n2c3ccccc3c3ccccc32)nc(-n2c3ccccc3c3ccccc32)c1] 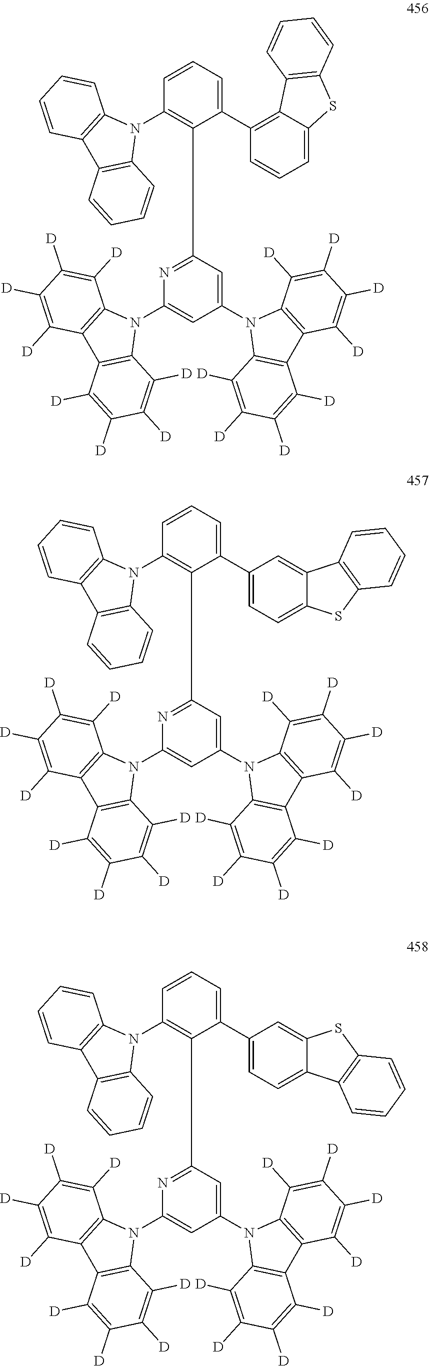 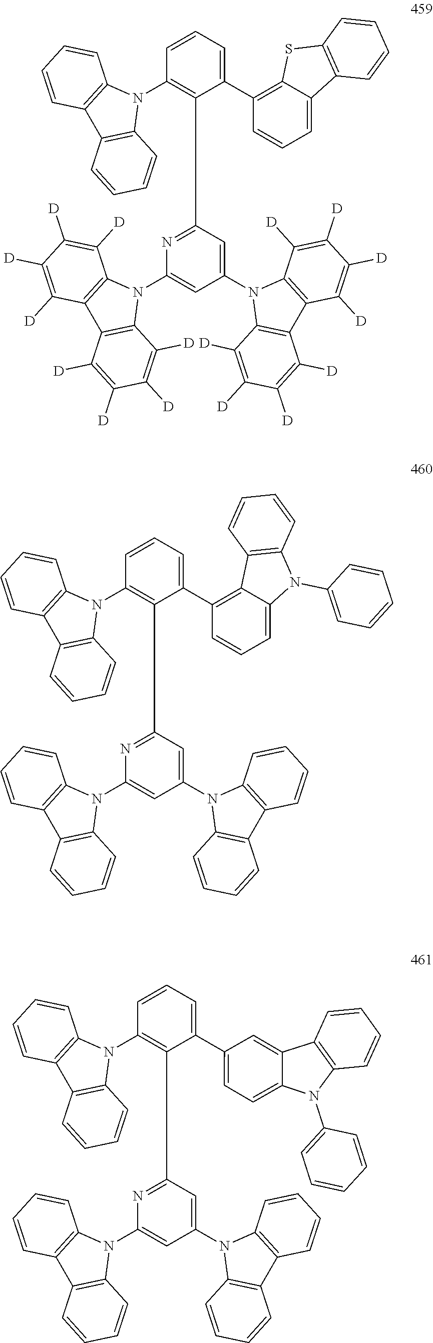 [SMILES: [2H]c1c([2H])c([2H])c2c(c1[2H])c1c([2H])c([2H])c([2H])c([2H])c1n2-c1cc(-c2c(-c3cccc4c3sc3ccccc34)cccc2-n2c3ccccc3c3ccccc32)nc(-n2c3c([2H])c([2H])c([2H])c([2H])c3c3c([2H])c([2H])c([2H])c([2H])c32)c1 ; c1ccc(-n2c3ccccc3c3c(-c4cccc(-n5c6ccccc6c6ccccc65)c4-c4cc(-n5c6ccccc6c6ccccc65)cc(-n5c6ccccc6c6ccccc65)n4)cccc32)cc1 ; c1ccc(-n2c3ccccc3c3cc(-c4cccc(-n5c6ccccc6c6ccccc65)c4-c4cc(-n5c6ccccc6c6ccccc65)cc(-n5c6ccccc6c6ccccc65)n4)ccc32)cc1] 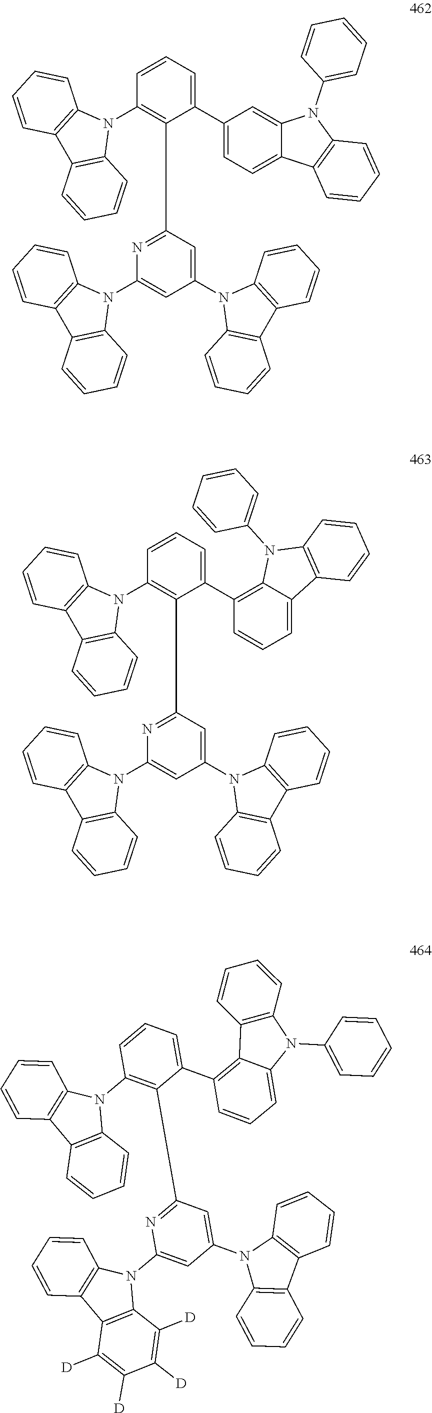 [SMILES: c1ccc(-n2c3ccccc3c3ccc(-c4cccc(-n5c6ccccc6c6ccccc65)c4-c4cc(-n5c6ccccc6c6ccccc65)cc(-n5c6ccccc6c6ccccc65)n4)cc32)cc1 ; c1ccc(-n2c3ccccc3c3cccc(-c4cccc(-n5c6ccccc6c6ccccc65)c4-c4cc(-n5c6ccccc6c6ccccc65)cc(-n5c6ccccc6c6ccccc65)n4)c32)cc1 ; [2H]c1c([2H])c([2H])c2c(c1[2H])c1ccccc1n2-c1cc(-n2c3ccccc3c3ccccc32)cc(-c2c(-c3cccc4c3c3ccccc3n4-c3ccccc3)cccc2-n2c3ccccc3c3ccccc32)n1] 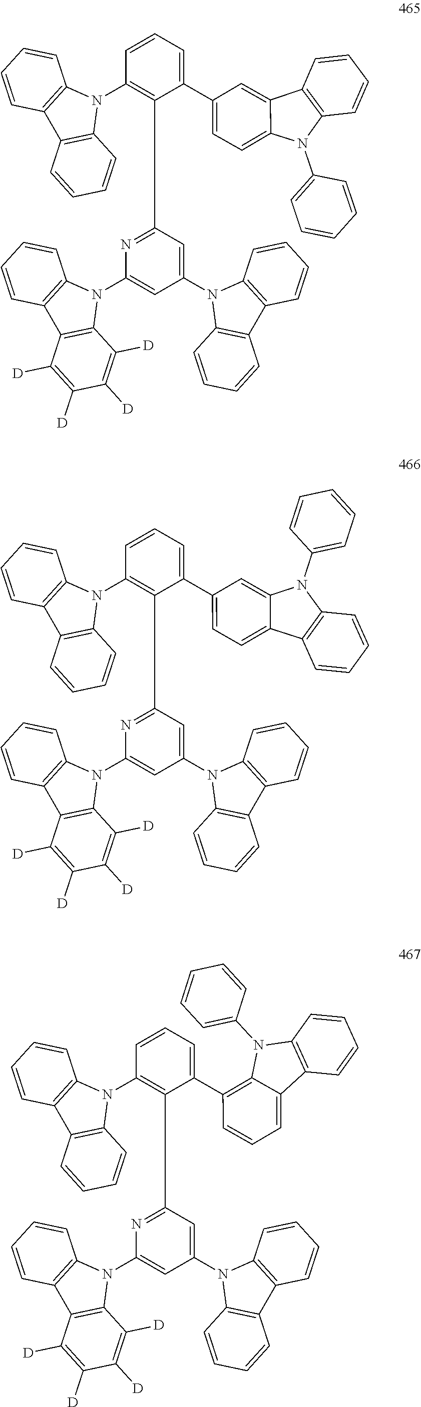 [SMILES: [2H]c1c([2H])c([2H])c2c(c1[2H])c1ccccc1n2-c1cc(-n2c3ccccc3c3ccccc32)cc(-c2c(-c3ccc4c(c3)c3ccccc3n4-c3ccccc3)cccc2-n2c3ccccc3c3ccccc32)n1 ; [2H]c1c([2H])c([2H])c2c(c1[2H])c1ccccc1n2-c1cc(-n2c3ccccc3c3ccccc32)cc(-c2c(-c3ccc4c5ccccc5n(-c5ccccc5)c4c3)cccc2-n2c3ccccc3c3ccccc32)n1 ; [2H]c1c([2H])c([2H])c2c(c1[2H])c1ccccc1n2-c1cc(-n2c3ccccc3c3ccccc32)cc(-c2c(-c3cccc4c5ccccc5n(-c5ccccc5)c34)cccc2-n2c3ccccc3c3ccccc32)n1] 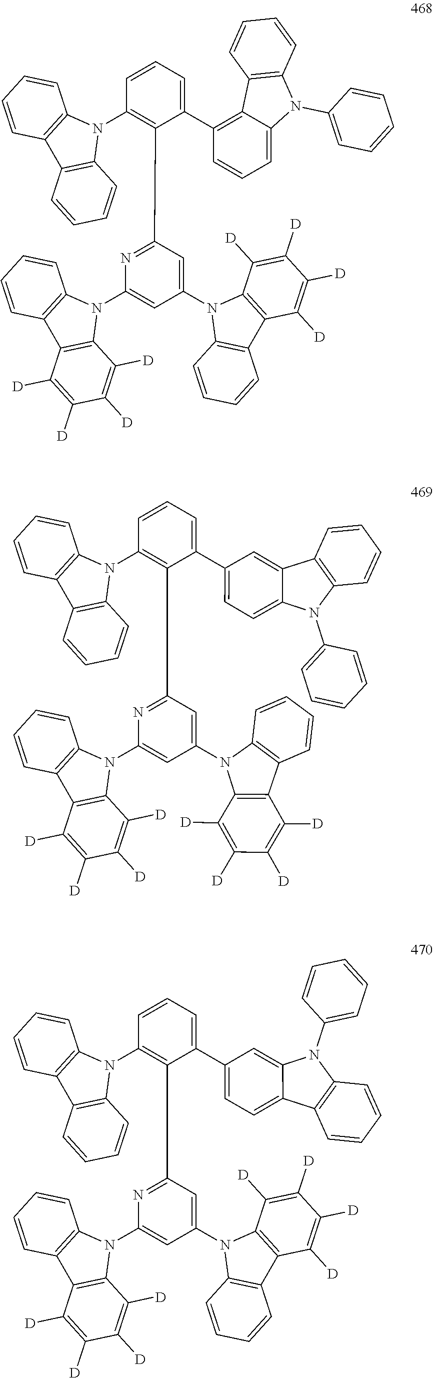 [SMILES: [2H]c1c([2H])c([2H])c2c(c1[2H])c1ccccc1n2-c1cc(-c2c(-c3cccc4c3c3ccccc3n4-c3ccccc3)cccc2-n2c3ccccc3c3ccccc32)nc(-n2c3ccccc3c3c([2H])c([2H])c([2H])c([2H])c32)c1 ; [2H]c1c([2H])c([2H])c2c(c1[2H])c1ccccc1n2-c1cc(-c2c(-c3ccc4c(c3)c3ccccc3n4-c3ccccc3)cccc2-n2c3ccccc3c3ccccc32)nc(-n2c3ccccc3c3c([2H])c([2H])c([2H])c([2H])c32)c1 ; [2H]c1c([2H])c([2H])c2c(c1[2H])c1ccccc1n2-c1cc(-c2c(-c3ccc4c5ccccc5n(-c5ccccc5)c4c3)cccc2-n2c3ccccc3c3ccccc32)nc(-n2c3ccccc3c3c([2H])c([2H])c([2H])c([2H])c32)c1] 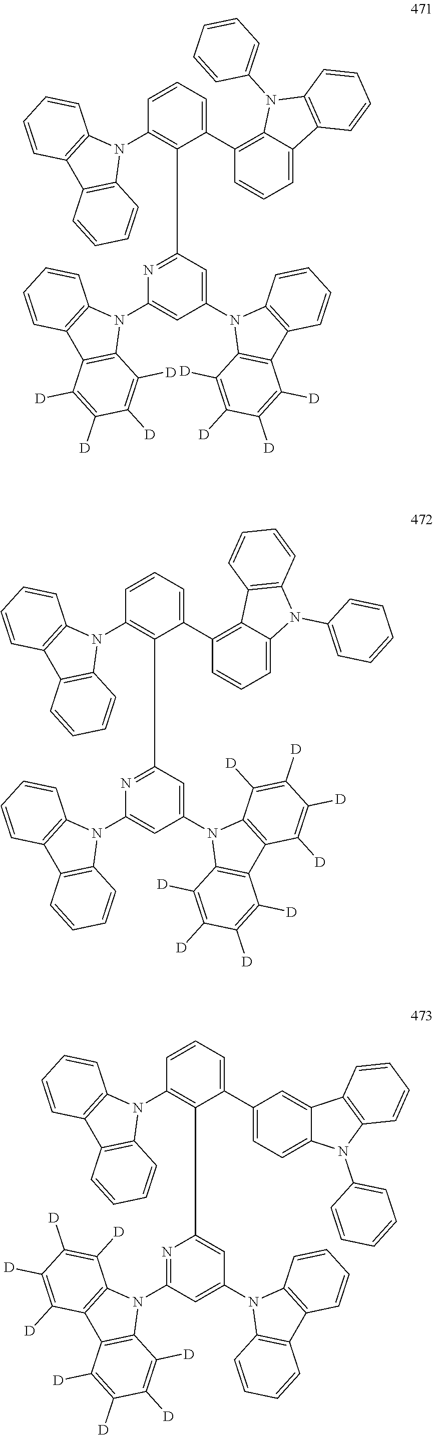 [SMILES: [2H]c1c([2H])c([2H])c2c(c1[2H])c1ccccc1n2-c1cc(-c2c(-c3cccc4c5ccccc5n(-c5ccccc5)c34)cccc2-n2c3ccccc3c3ccccc32)nc(-n2c3ccccc3c3c([2H])c([2H])c([2H])c([2H])c32)c1 ; [2H]c1c([2H])c([2H])c2c(c1[2H])c1c([2H])c([2H])c([2H])c([2H])c1n2-c1cc(-c2c(-c3cccc4c3c3ccccc3n4-c3ccccc3)cccc2-n2c3ccccc3c3ccccc32)nc(-n2c3ccccc3c3ccccc32)c1 ; [2H]c1c([2H])c([2H])c2c(c1[2H])c1c([2H])c([2H])c([2H])c([2H])c1n2-c1cc(-n2c3ccccc3c3ccccc32)cc(-c2c(-c3ccc4c(c3)c3ccccc3n4-c3ccccc3)cccc2-n2c3ccccc3c3ccccc32)n1] 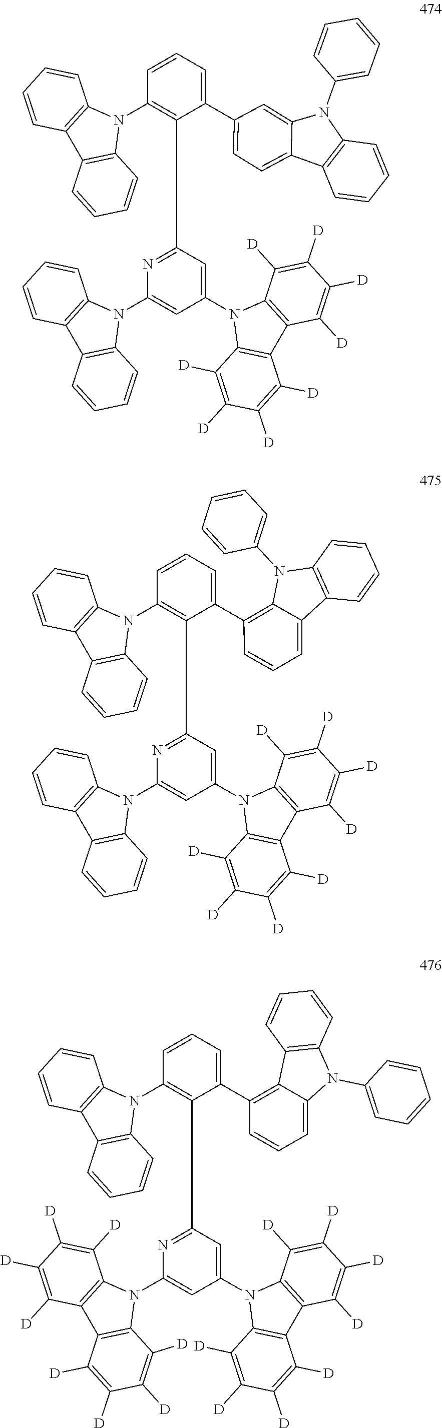 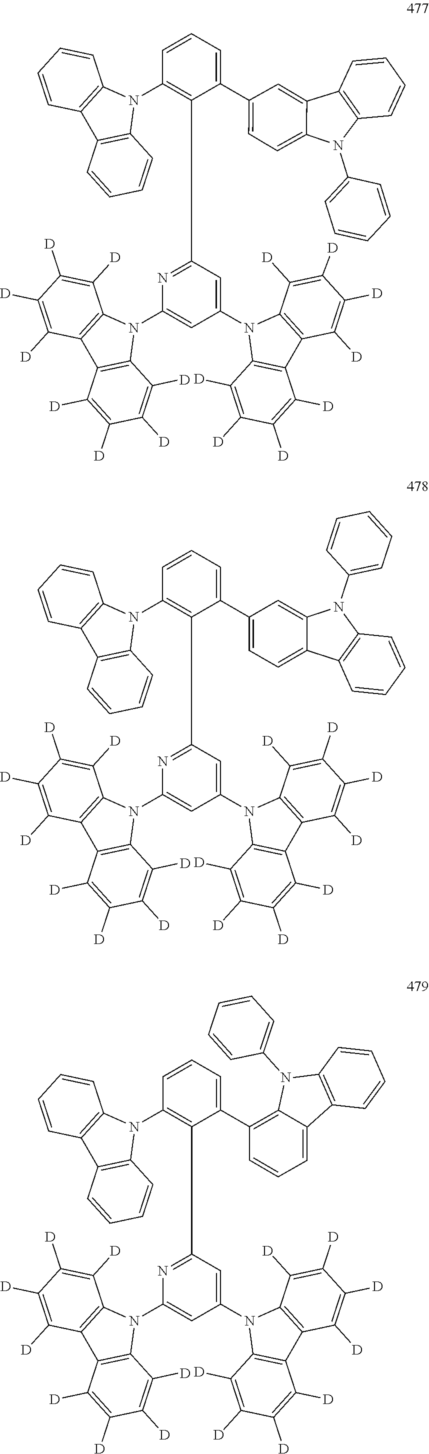 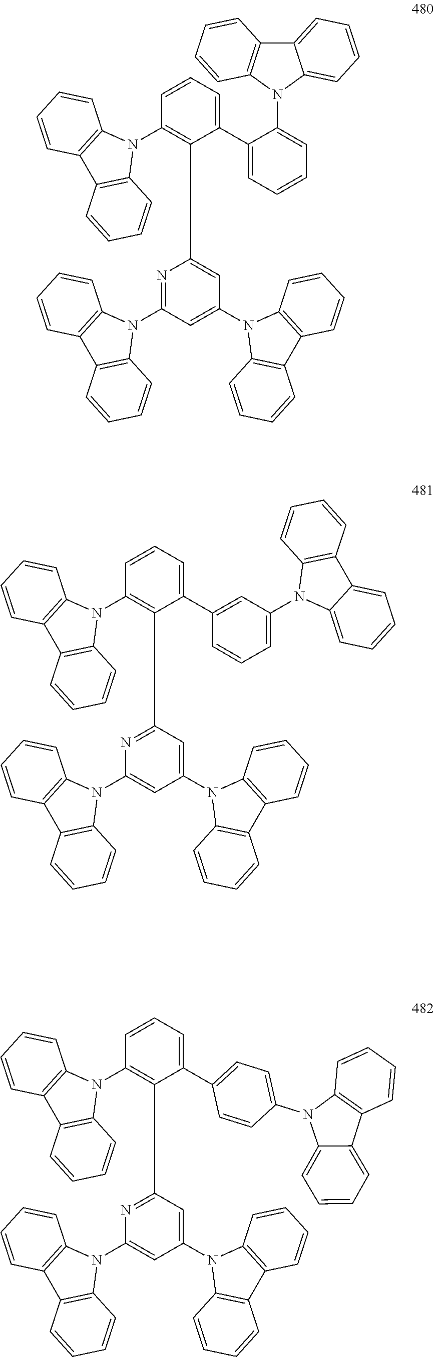 [SMILES: c1ccc(-n2c3ccccc3c3ccccc32)c(-c2cccc(-n3c4ccccc4c4ccccc43)c2-c2cc(-n3c4ccccc4c4ccccc43)cc(-n3c4ccccc4c4ccccc43)n2)c1 ; c1cc(-c2cccc(-n3c4ccccc4c4ccccc43)c2-c2cc(-n3c4ccccc4c4ccccc43)cc(-n3c4ccccc4c4ccccc43)n2)cc(-n2c3ccccc3c3ccccc32)c1 ; c1cc(-c2ccc(-n3c4ccccc4c4ccccc43)cc2)c(-c2cc(-n3c4ccccc4c4ccccc43)cc(-n3c4ccccc4c4ccccc43)n2)c(-n2c3ccccc3c3ccccc32)c1] 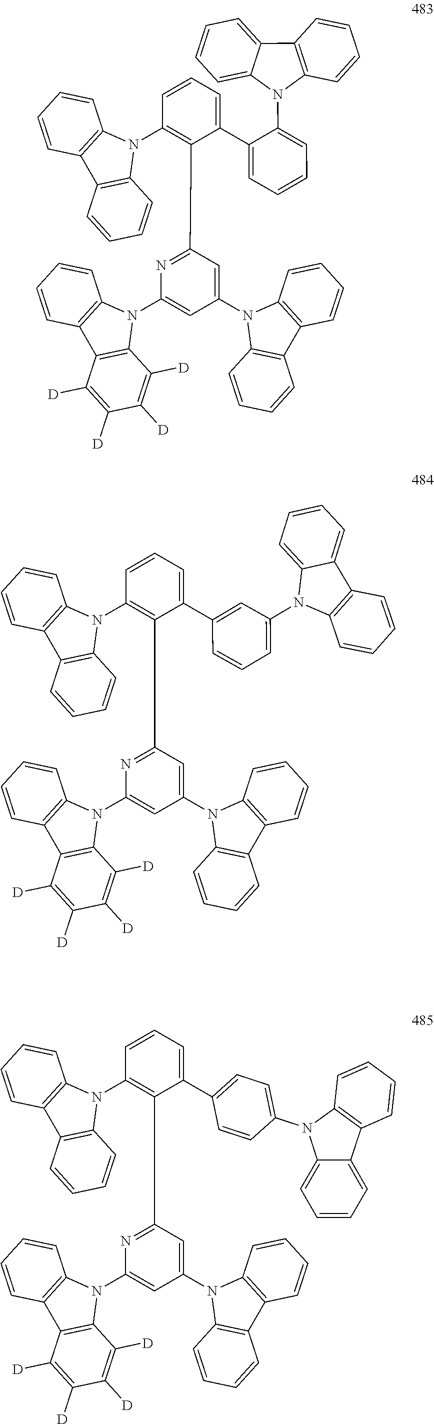 [SMILES: [2H]c1c([2H])c([2H])c2c(c1[2H])c1ccccc1n2-c1cc(-n2c3ccccc3c3ccccc32)cc(-c2c(-c3ccccc3-n3c4ccccc4c4ccccc43)cccc2-n2c3ccccc3c3ccccc32)n1 ; [2H]c1c([2H])c([2H])c2c(c1[2H])c1ccccc1n2-c1cc(-n2c3ccccc3c3ccccc32)cc(-c2c(-c3cccc(-n4c5ccccc5c5ccccc54)c3)cccc2-n2c3ccccc3c3ccccc32)n1 ; [2H]c1c([2H])c([2H])c2c(c1[2H])c1ccccc1n2-c1cc(-n2c3ccccc3c3ccccc32)cc(-c2c(-c3ccc(-n4c5ccccc5c5ccccc54)cc3)cccc2-n2c3ccccc3c3ccccc32)n1] 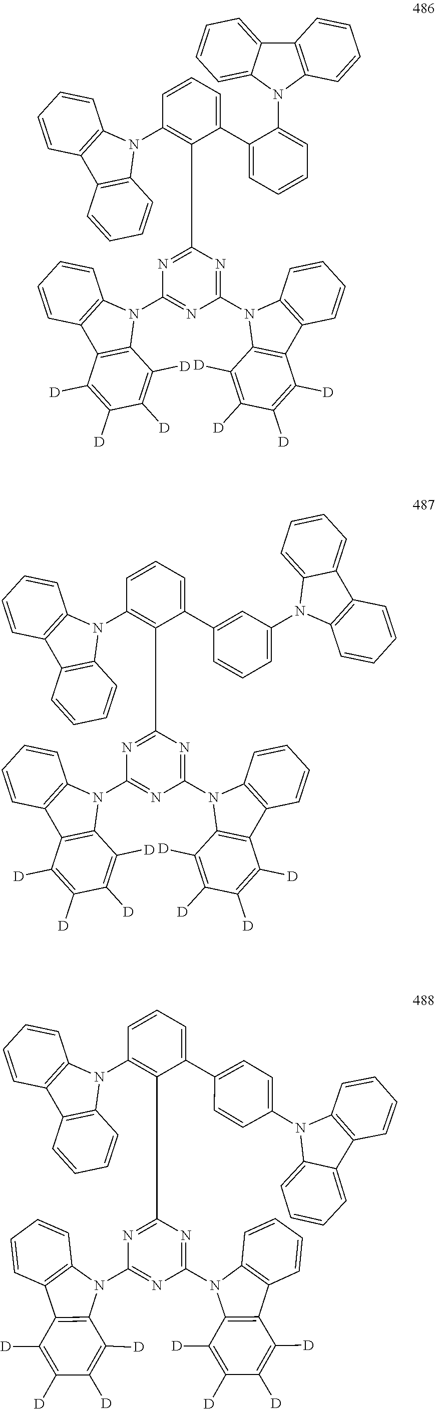 [SMILES: [2H]c1c([2H])c([2H])c2c(c1[2H])c1ccccc1n2-c1nc(-c2c(-c3ccccc3-n3c4ccccc4c4ccccc43)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2c(-c3cccc(-n4c5ccccc5c5ccccc54)c3)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2c(-c3ccc(-n4c5ccccc5c5ccccc54)cc3)cccc2-n2c3ccccc3c3ccccc32)nc(-n2c3ccccc3c3c([2H])c([2H])c([2H])c([2H])c32)n1] 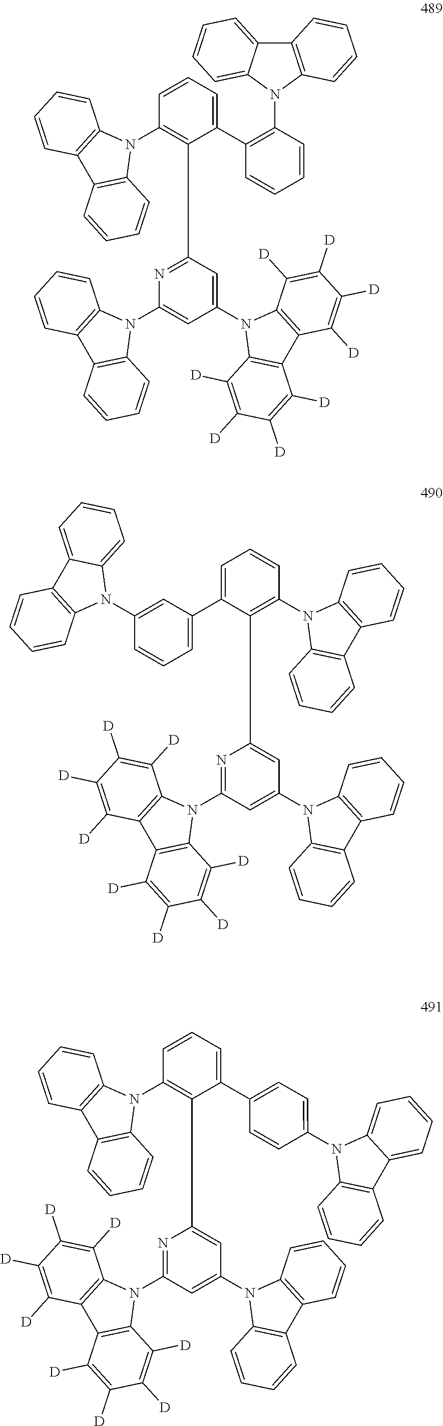 [SMILES: [2H]c1c([2H])c([2H])c2c(c1[2H])c1c([2H])c([2H])c([2H])c([2H])c1n2-c1cc(-c2c(-c3ccccc3-n3c4ccccc4c4ccccc43)cccc2-n2c3ccccc3c3ccccc32)nc(-n2c3ccccc3c3ccccc32)c1 ; [2H]c1c([2H])c([2H])c2c(c1[2H])c1c([2H])c([2H])c([2H])c([2H])c1n2-c1cc(-n2c3ccccc3c3ccccc32)cc(-c2c(-c3cccc(-n4c5ccccc5c5ccccc54)c3)cccc2-n2c3ccccc3c3ccccc32)n1 ; [2H]c1c([2H])c([2H])c2c(c1[2H])c1c([2H])c([2H])c([2H])c([2H])c1n2-c1cc(-n2c3ccccc3c3ccccc32)cc(-c2c(-c3ccc(-n4c5ccccc5c5ccccc54)cc3)cccc2-n2c3ccccc3c3ccccc32)n1] 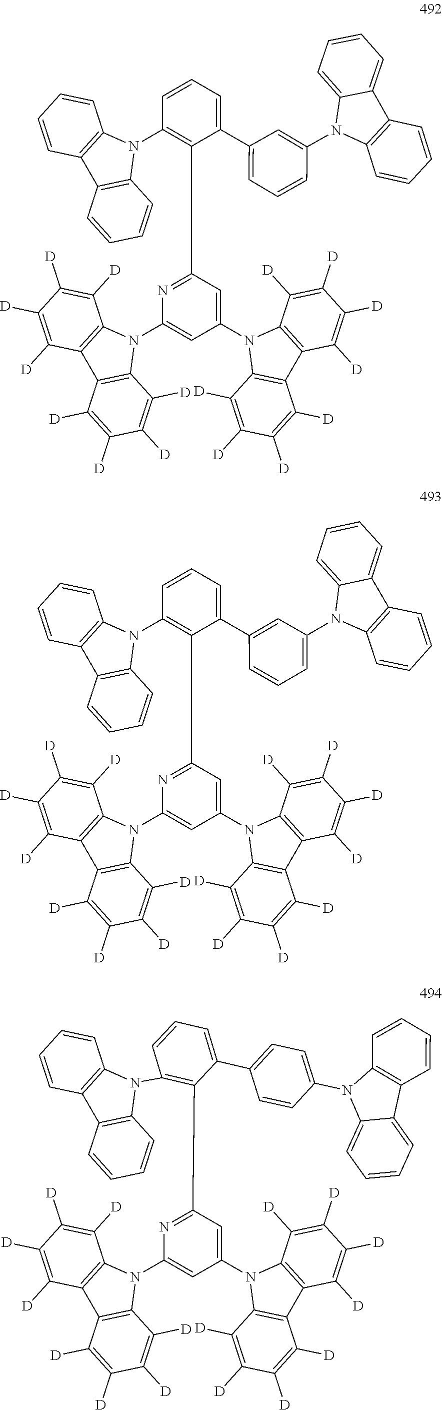 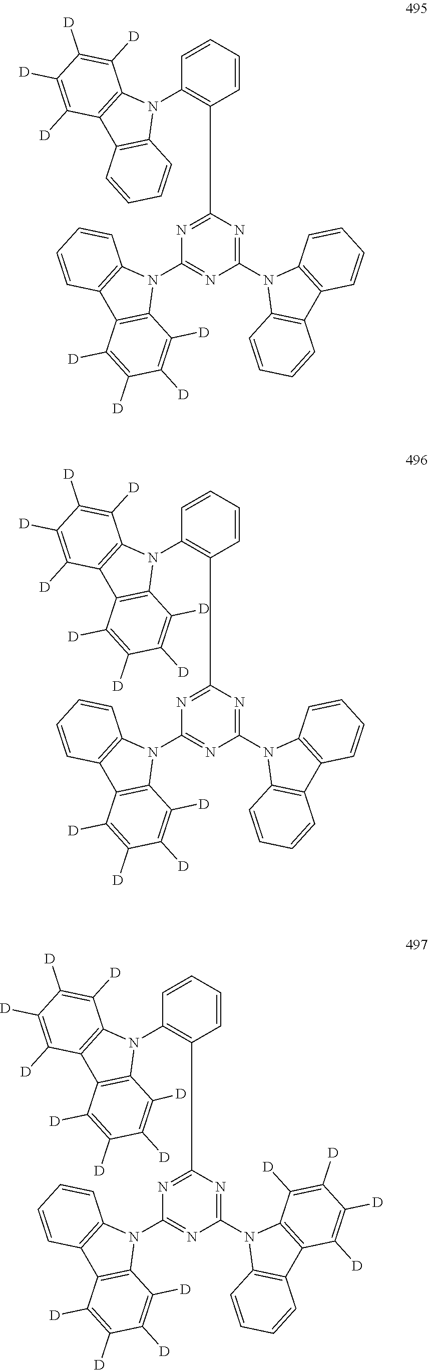 [SMILES: [2H]c1c([2H])c([2H])c2c(c1[2H])c1ccccc1n2-c1nc(-c2ccccc2-n2c3ccccc3c3c([2H])c([2H])c([2H])c([2H])c32)nc(-n2c3ccccc3c3ccccc32)n1 ; [2H]c1c([2H])c([2H])c2c(c1[2H])c1ccccc1n2-c1nc(-c2ccccc2-n2c3c([2H])c([2H])c([2H])c([2H])c3c3c([2H])c([2H])c([2H])c([2H])c32)nc(-n2c3ccccc3c3ccccc32)n1 ; [2H]c1c([2H])c([2H])c2c(c1[2H])c1ccccc1n2-c1nc(-c2ccccc2-n2c3c([2H])c([2H])c([2H])c([2H])c3c3c([2H])c([2H])c([2H])c([2H])c32)nc(-n2c3ccccc3c3c([2H])c([2H])c([2H])c([2H])c32)n1] 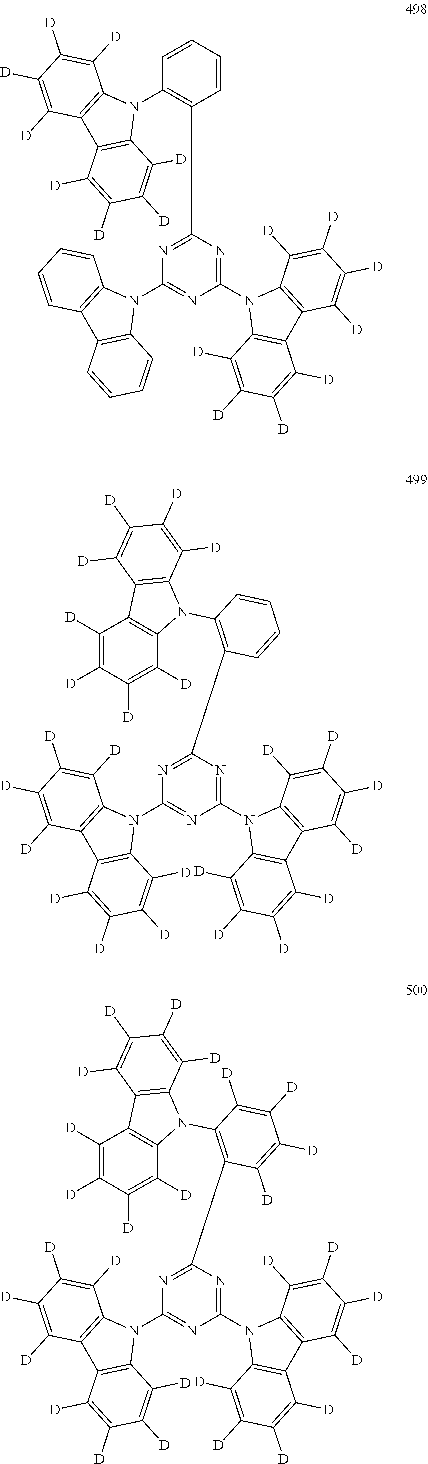 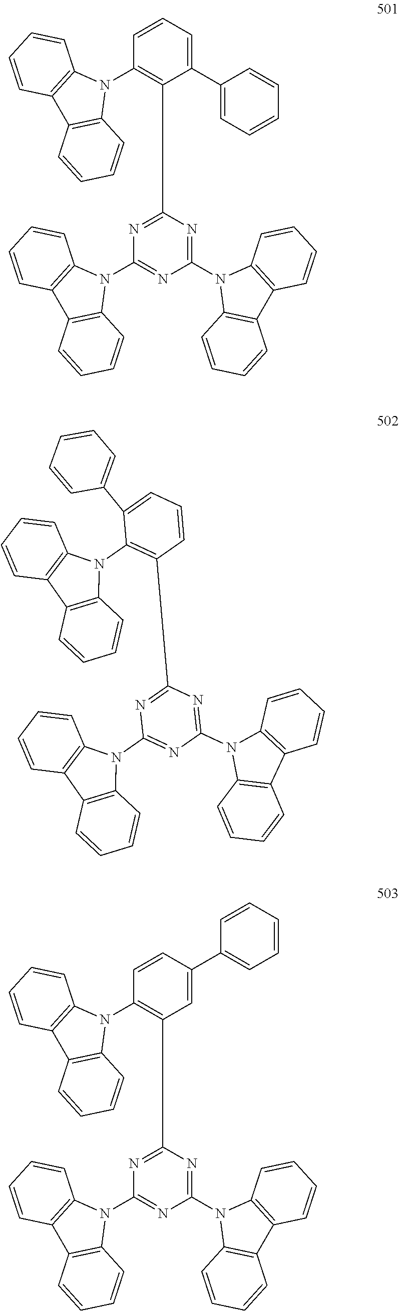 [SMILES: c1ccc(-c2cccc(-n3c4ccccc4c4ccccc43)c2-c2nc(-n3c4ccccc4c4ccccc43)nc(-n3c4ccccc4c4ccccc43)n2)cc1 ; c1ccc(-c2cccc(-c3nc(-n4c5ccccc5c5ccccc54)nc(-n4c5ccccc5c5ccccc54)n3)c2-n2c3ccccc3c3ccccc32)cc1 ; c1ccc(-c2ccc(-n3c4ccccc4c4ccccc43)c(-c3nc(-n4c5ccccc5c5ccccc54)nc(-n4c5ccccc5c5ccccc54)n3)c2)cc1] 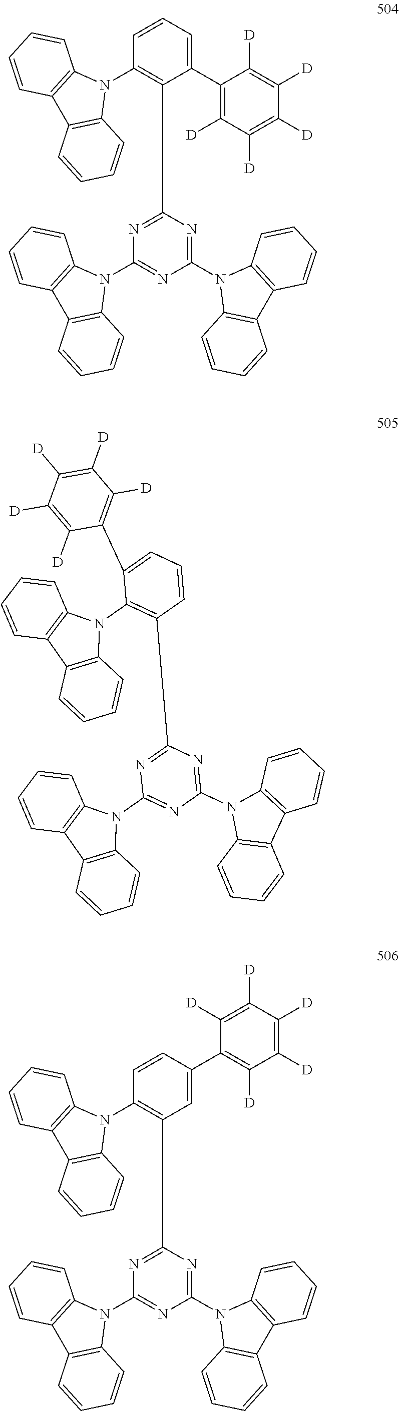 [SMILES: [2H]c1c([2H])c([2H])c(-c2cccc(-n3c4ccccc4c4ccccc43)c2-c2nc(-n3c4ccccc4c4ccccc43)nc(-n3c4ccccc4c4ccccc43)n2)c([2H])c1[2H] ; [2H]c1c([2H])c([2H])c(-c2cccc(-c3nc(-n4c5ccccc5c5ccccc54)nc(-n4c5ccccc5c5ccccc54)n3)c2-n2c3ccccc3c3ccccc32)c([2H])c1[2H] ; [2H]c1c([2H])c([2H])c(-c2ccc(-n3c4ccccc4c4ccccc43)c(-c3nc(-n4c5ccccc5c5ccccc54)nc(-n4c5ccccc5c5ccccc54)n3)c2)c([2H])c1[2H]] 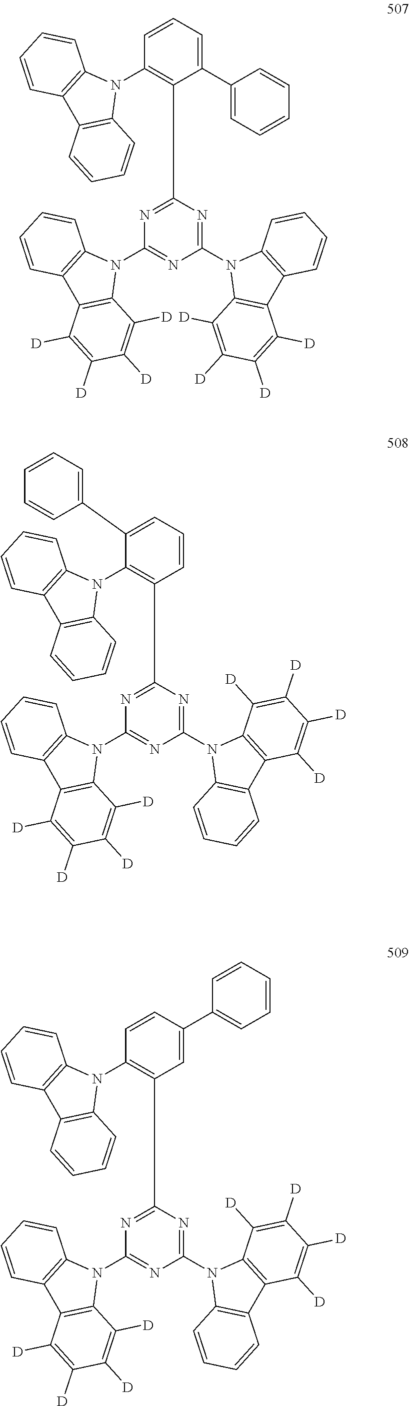 [SMILES: [2H]c1c([2H])c([2H])c2c(c1[2H])c1ccccc1n2-c1nc(-c2c(-c3ccccc3)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2cccc(-c3ccccc3)c2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2cc(-c3ccccc3)ccc2-n2c3ccccc3c3ccccc32)nc(-n2c3ccccc3c3c([2H])c([2H])c([2H])c([2H])c32)n1] 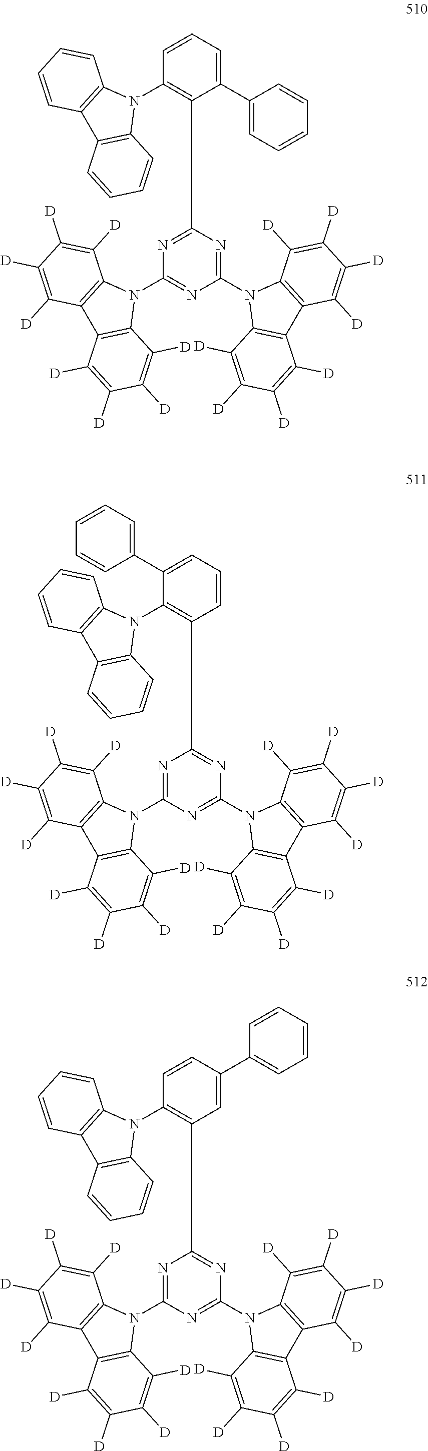 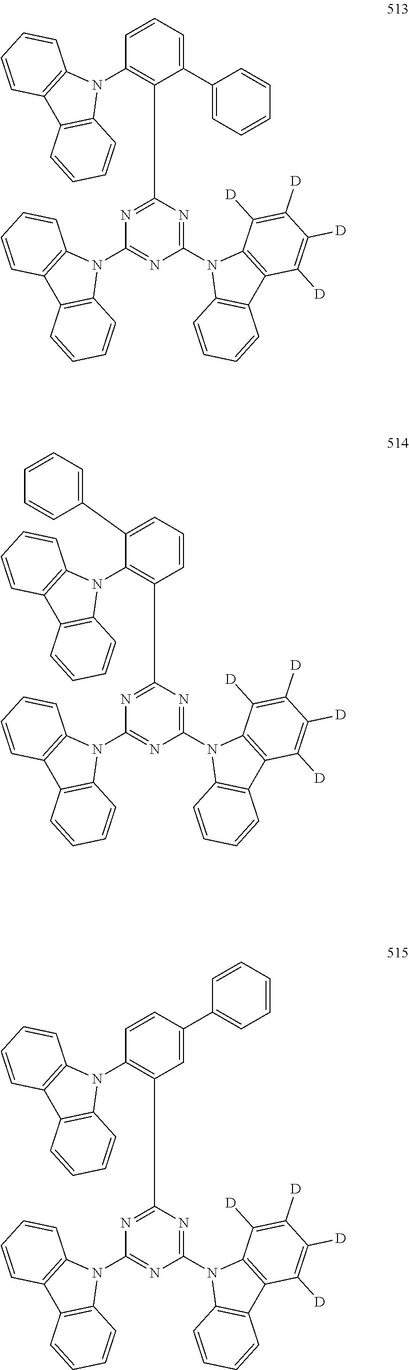 [SMILES: [2H]c1c([2H])c([2H])c2c(c1[2H])c1ccccc1n2-c1nc(-c2c(-c3ccccc3)cccc2-n2c3ccccc3c3ccccc32)nc(-n2c3ccccc3c3ccccc32)n1 ; [2H]c1c([2H])c([2H])c2c(c1[2H])c1ccccc1n2-c1nc(-c2cccc(-c3ccccc3)c2-n2c3ccccc3c3ccccc32)nc(-n2c3ccccc3c3ccccc32)n1 ; [2H]c1c([2H])c([2H])c2c(c1[2H])c1ccccc1n2-c1nc(-c2cc(-c3ccccc3)ccc2-n2c3ccccc3c3ccccc32)nc(-n2c3ccccc3c3ccccc32)n1] 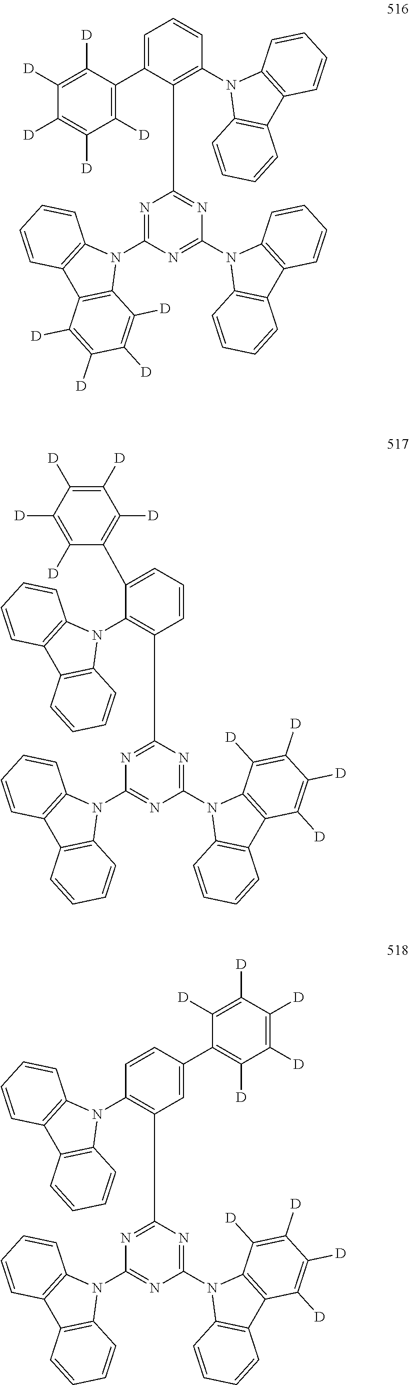 [SMILES: [2H]c1c([2H])c([2H])c(-c2cccc(-n3c4ccccc4c4ccccc43)c2-c2nc(-n3c4ccccc4c4ccccc43)nc(-n3c4ccccc4c4c([2H])c([2H])c([2H])c([2H])c43)n2)c([2H])c1[2H] ; [2H]c1c([2H])c([2H])c(-c2cccc(-c3nc(-n4c5ccccc5c5ccccc54)nc(-n4c5ccccc5c5c([2H])c([2H])c([2H])c([2H])c54)n3)c2-n2c3ccccc3c3ccccc32)c([2H])c1[2H] ; [2H]c1c([2H])c([2H])c(-c2ccc(-n3c4ccccc4c4ccccc43)c(-c3nc(-n4c5ccccc5c5ccccc54)nc(-n4c5ccccc5c5c([2H])c([2H])c([2H])c([2H])c54)n3)c2)c([2H])c1[2H]] 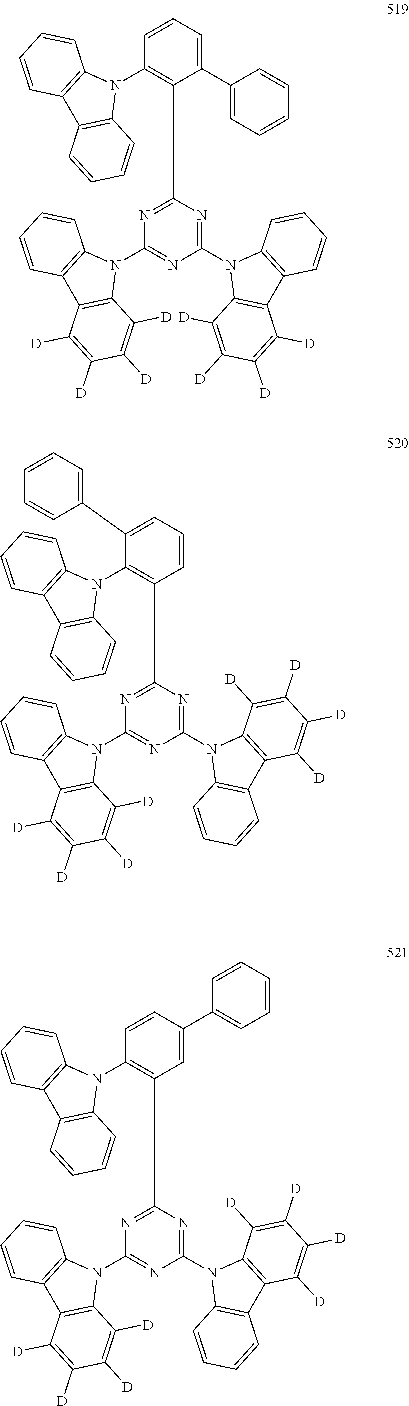 [SMILES: [2H]c1c([2H])c([2H])c2c(c1[2H])c1ccccc1n2-c1nc(-c2c(-c3ccccc3)cccc2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2cccc(-c3ccccc3)c2-n2c3ccccc3c3ccccc32)nc(-n2c3ccccc3c3c([2H])c([2H])c([2H])c([2H])c32)n1 ; [2H]c1c([2H])c([2H])c2c(c1[2H])c1ccccc1n2-c1nc(-c2cc(-c3ccccc3)ccc2-n2c3ccccc3c3ccccc32)nc(-n2c3ccccc3c3c([2H])c([2H])c([2H])c([2H])c32)n1] 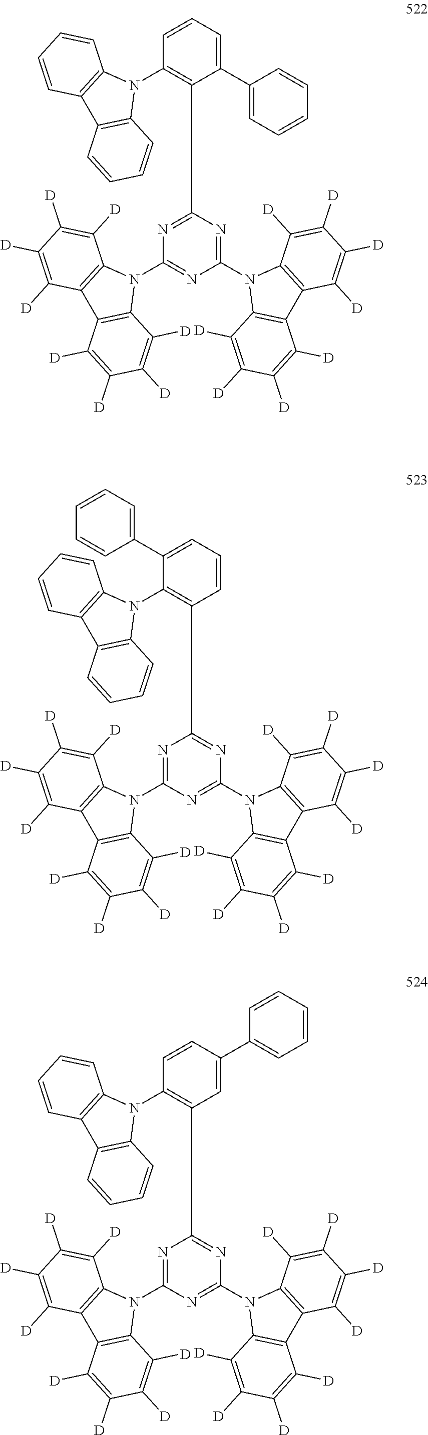 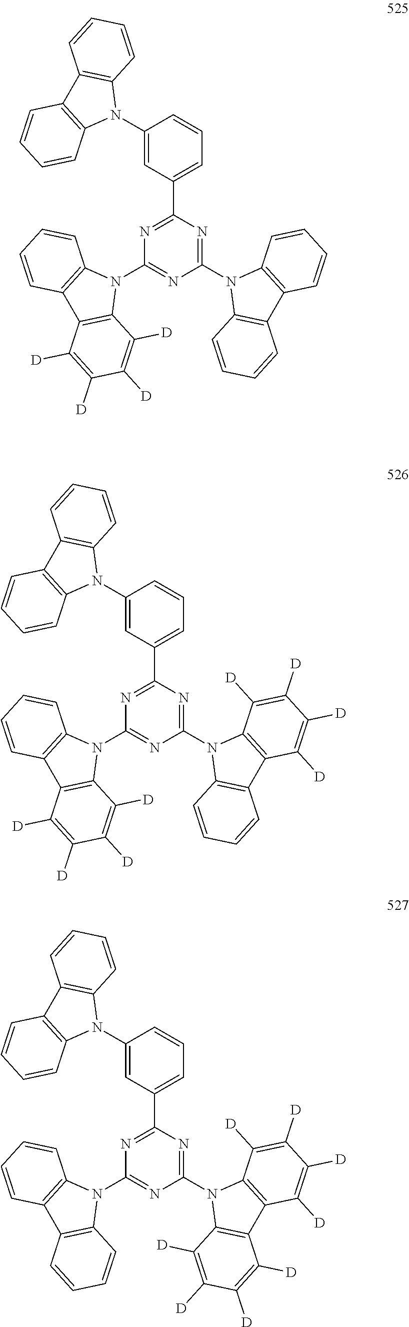 [SMILES: [2H]c1c([2H])c([2H])c2c(c1[2H])c1ccccc1n2-c1nc(-c2cccc(-n3c4ccccc4c4ccccc43)c2)nc(-n2c3ccccc3c3ccccc32)n1 ; [2H]c1c([2H])c([2H])c2c(c1[2H])c1ccccc1n2-c1nc(-c2cccc(-n3c4ccccc4c4ccccc43)c2)nc(-n2c3ccccc3c3c([2H])c([2H])c([2H])c([2H])c32)n1 ; [2H]c1c([2H])c([2H])c2c(c1[2H])c1c([2H])c([2H])c([2H])c([2H])c1n2-c1nc(-c2cccc(-n3c4ccccc4c4ccccc43)c2)nc(-n2c3ccccc3c3ccccc32)n1] 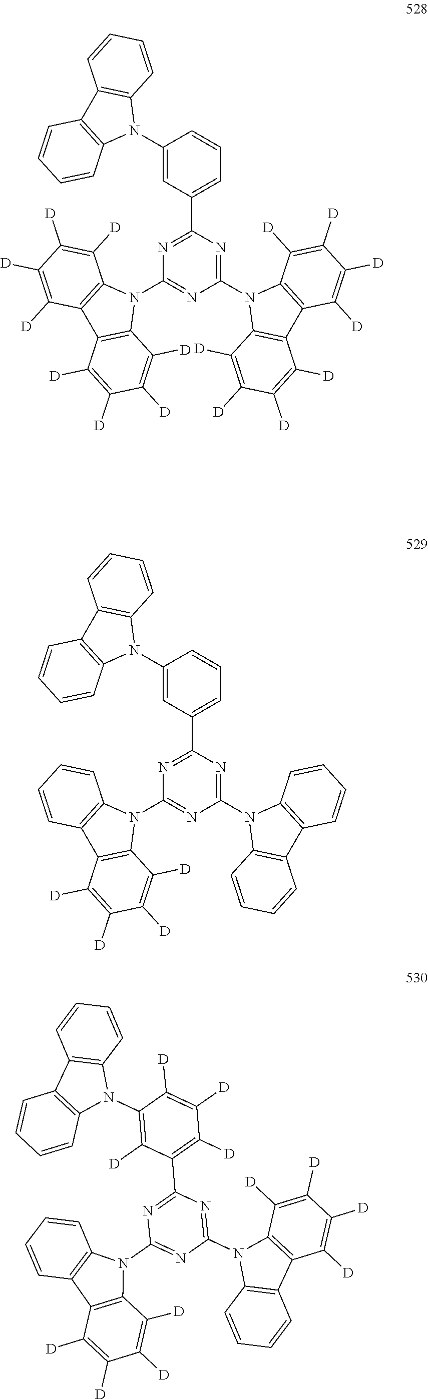 [SMILES: [2H]c1c([2H])c([2H])c2c(c1[2H])c1c([2H])c([2H])c([2H])c([2H])c1n2-c1nc(-c2cccc(-n3c4ccccc4c4ccccc43)c2)nc(-n2c3c([2H])c([2H])c([2H])c([2H])c3c3c([2H])c([2H])c([2H])c([2H])c32)n1 ; [2H]c1c([2H])c([2H])c2c(c1[2H])c1ccccc1n2-c1nc(-c2cccc(-n3c4ccccc4c4ccccc43)c2)nc(-n2c3ccccc3c3ccccc32)n1 ; [2H]c1c([2H])c(-c2nc(-n3c4ccccc4c4c([2H])c([2H])c([2H])c([2H])c43)nc(-n3c4ccccc4c4c([2H])c([2H])c([2H])c([2H])c43)n2)c([2H])c(-n2c3ccccc3c3ccccc32)c1[2H]] 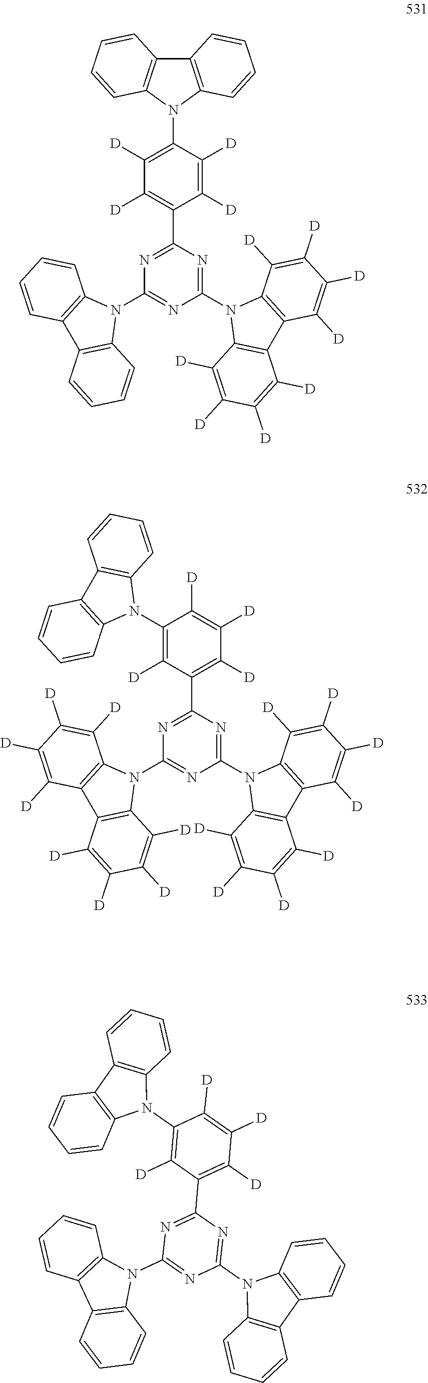 [SMILES: [2H]c1c([2H])c(-n2c3ccccc3c3ccccc32)c([2H])c([2H])c1-c1nc(-n2c3ccccc3c3ccccc32)nc(-n2c3c([2H])c([2H])c([2H])c([2H])c3c3c([2H])c([2H])c([2H])c([2H])c32)n1 ; [2H]c1c([2H])c(-c2nc(-n3c4c([2H])c([2H])c([2H])c([2H])c4c4c([2H])c([2H])c([2H])c([2H])c43)nc(-n3c4c([2H])c([2H])c([2H])c([2H])c4c4c([2H])c([2H])c([2H])c([2H])c43)n2)c([2H])c(-n2c3ccccc3c3ccccc32)c1[2H] ; [2H]c1c([2H])c(-c2nc(-n3c4ccccc4c4ccccc43)nc(-n3c4ccccc4c4ccccc43)n2)c([2H])c(-n2c3ccccc3c3ccccc32)c1[2H]] 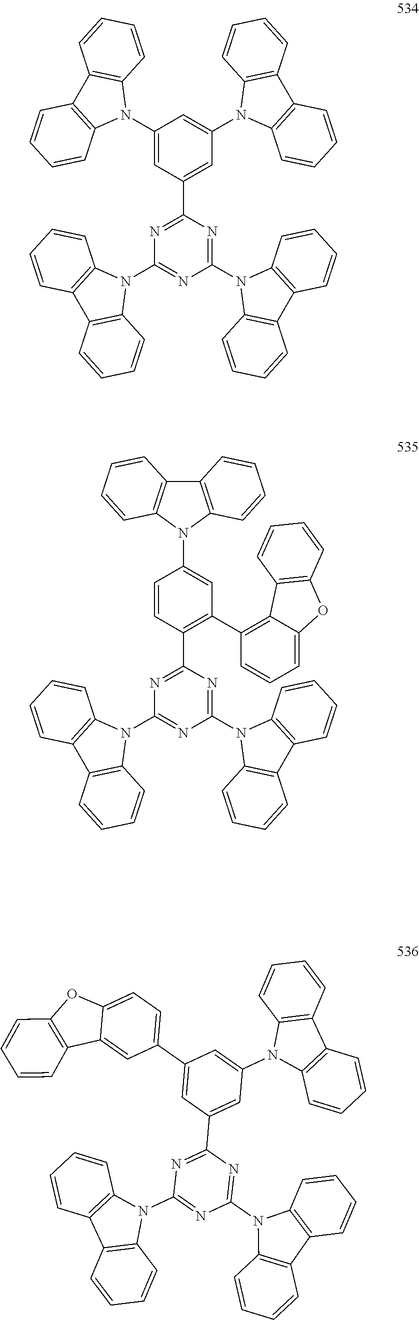 [SMILES: c1ccc2c(c1)c1ccccc1n2-c1cc(-c2nc(-n3c4ccccc4c4ccccc43)nc(-n3c4ccccc4c4ccccc43)n2)cc(-n2c3ccccc3c3ccccc32)c1 ; c1ccc2c(c1)oc1cccc(-c3cc(-n4c5ccccc5c5ccccc54)ccc3-c3nc(-n4c5ccccc5c5ccccc54)nc(-n4c5ccccc5c5ccccc54)n3)c12 ; c1ccc2c(c1)oc1ccc(-c3cc(-c4nc(-n5c6ccccc6c6ccccc65)nc(-n5c6ccccc6c6ccccc65)n4)cc(-n4c5ccccc5c5ccccc54)c3)cc12] 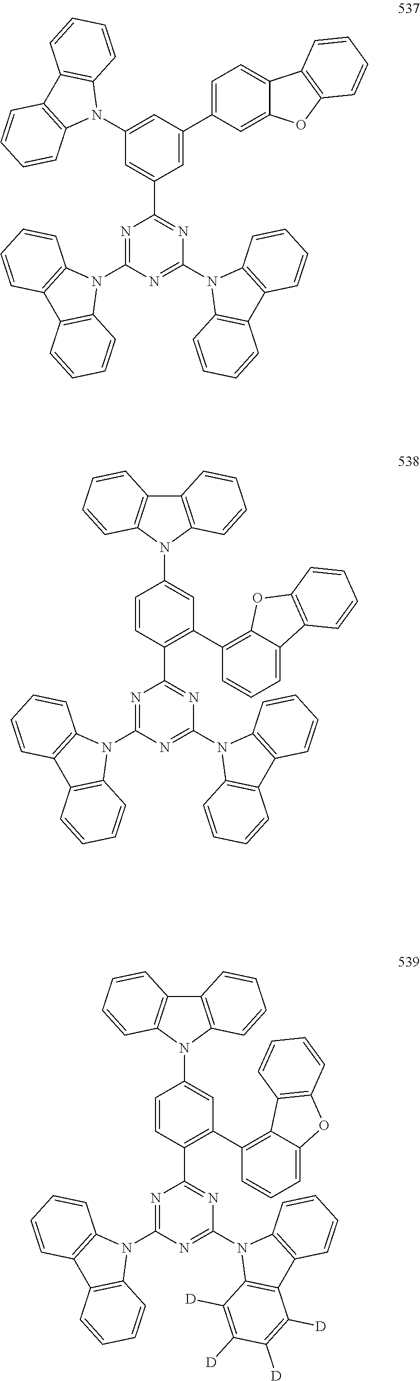 [SMILES: c1ccc2c(c1)oc1cc(-c3cc(-c4nc(-n5c6ccccc6c6ccccc65)nc(-n5c6ccccc6c6ccccc65)n4)cc(-n4c5ccccc5c5ccccc54)c3)ccc12 ; c1ccc2c(c1)oc1c(-c3cc(-n4c5ccccc5c5ccccc54)ccc3-c3nc(-n4c5ccccc5c5ccccc54)nc(-n4c5ccccc5c5ccccc54)n3)cccc12 ; [2H]c1c([2H])c([2H])c2c(c1[2H])c1ccccc1n2-c1nc(-c2ccc(-n3c4ccccc4c4ccccc43)cc2-c2cccc3oc4ccccc4c23)nc(-n2c3ccccc3c3ccccc32)n1] 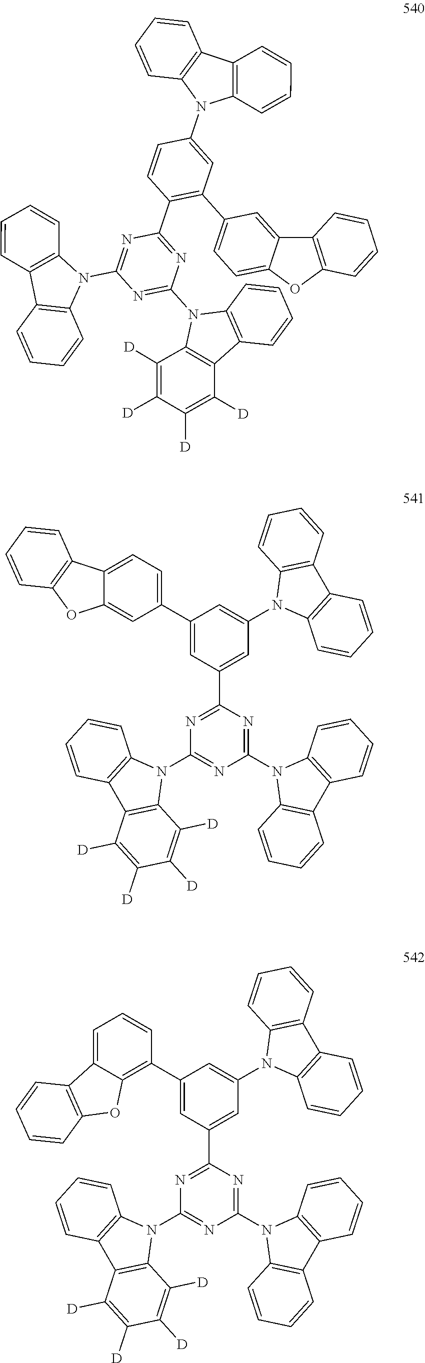 [SMILES: [2H]c1c([2H])c([2H])c2c(c1[2H])c1ccccc1n2-c1nc(-c2ccc(-n3c4ccccc4c4ccccc43)cc2-c2ccc3oc4ccccc4c3c2)nc(-n2c3ccccc3c3ccccc32)n1 ; [2H]c1c([2H])c([2H])c2c(c1[2H])c1ccccc1n2-c1nc(-c2cc(-c3ccc4c(c3)oc3ccccc34)cc(-n3c4ccccc4c4ccccc43)c2)nc(-n2c3ccccc3c3ccccc32)n1 ; [2H]c1c([2H])c([2H])c2c(c1[2H])c1ccccc1n2-c1nc(-c2cc(-c3cccc4c3oc3ccccc34)cc(-n3c4ccccc4c4ccccc43)c2)nc(-n2c3ccccc3c3ccccc32)n1] 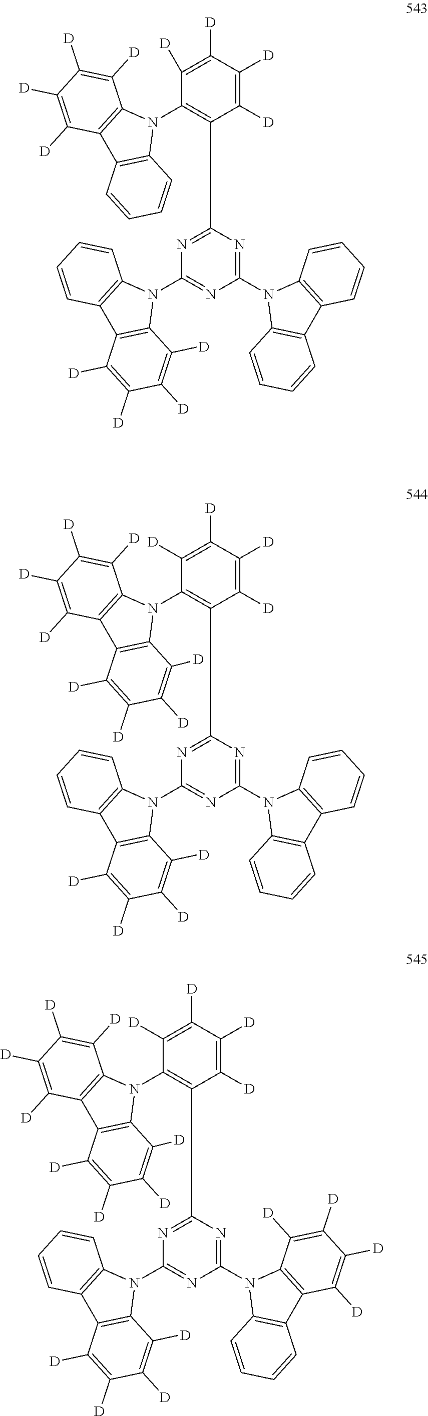 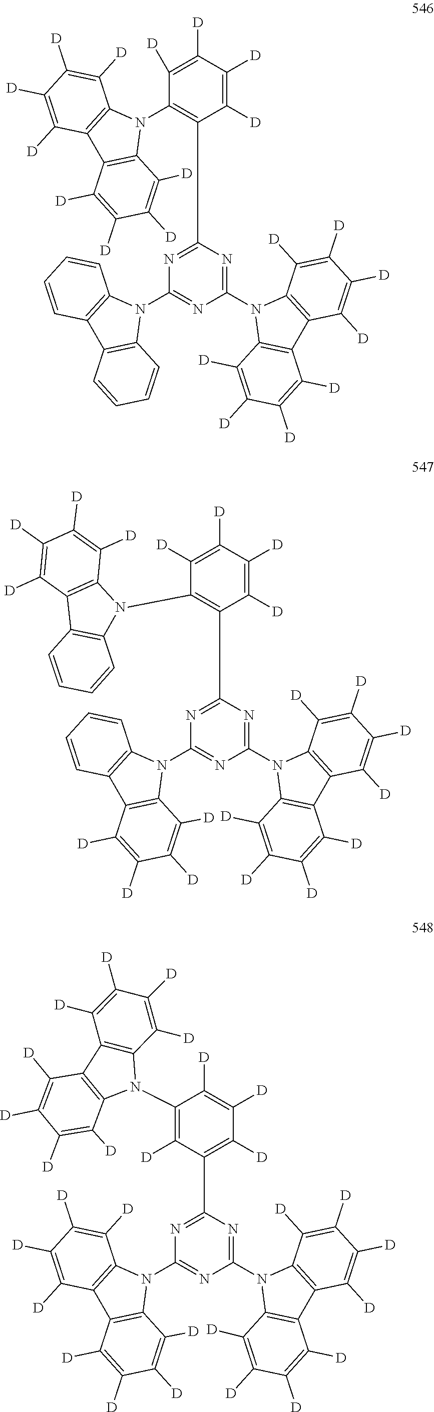 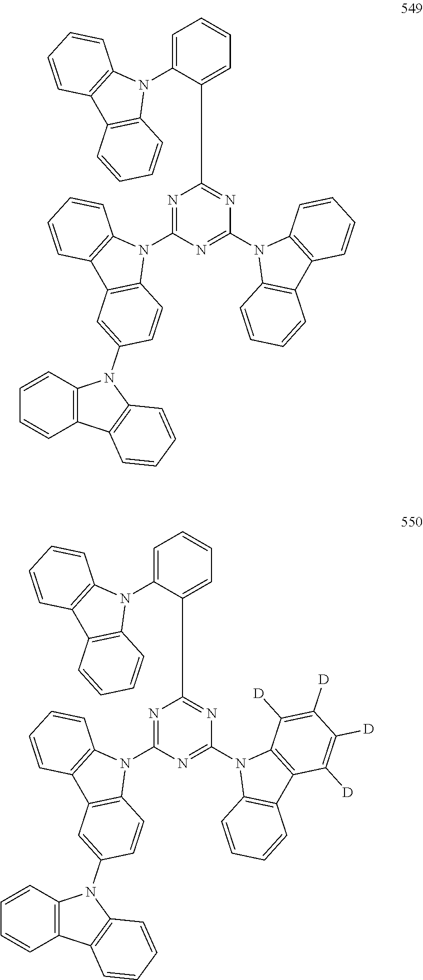 [SMILES: c1ccc(-n2c3ccccc3c3ccccc32)c(-c2nc(-n3c4ccccc4c4ccccc43)nc(-n3c4ccccc4c4cc(-n5c6ccccc6c6ccccc65)ccc43)n2)c1 ; [2H]c1c([2H])c([2H])c2c(c1[2H])c1ccccc1n2-c1nc(-c2ccccc2-n2c3ccccc3c3ccccc32)nc(-n2c3ccccc3c3cc(-n4c5ccccc5c5ccccc54)ccc32)n1] 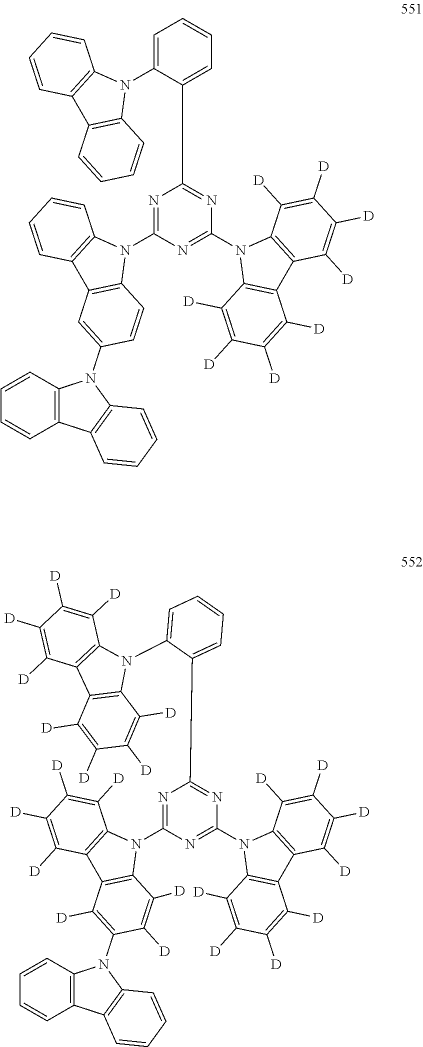 [SMILES: [2H]c1c([2H])c([2H])c2c(c1[2H])c1c([2H])c([2H])c([2H])c([2H])c1n2-c1nc(-c2ccccc2-n2c3ccccc3c3ccccc32)nc(-n2c3ccccc3c3cc(-n4c5ccccc5c5ccccc54)ccc32)n1 ; [2H]c1c([2H])c([2H])c2c(c1[2H])c1c([2H])c([2H])c([2H])c([2H])c1n2-c1nc(-c2ccccc2-n2c3c([2H])c([2H])c([2H])c([2H])c3c3c([2H])c([2H])c([2H])c([2H])c32)nc(-n2c3c([2H])c([2H])c([2H])c([2H])c3c3c([2H])c(-n4c5ccccc5c5ccccc54)c([2H])c([2H])c32)n1] 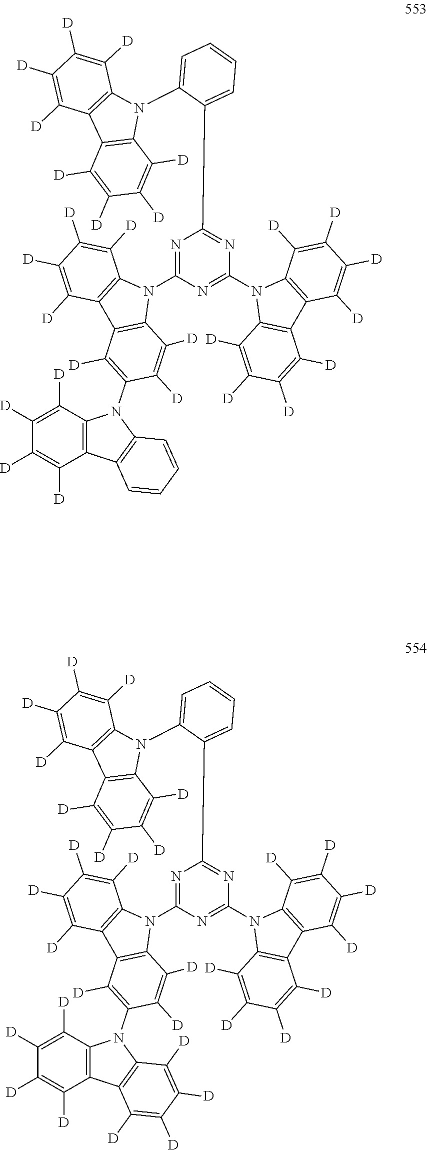 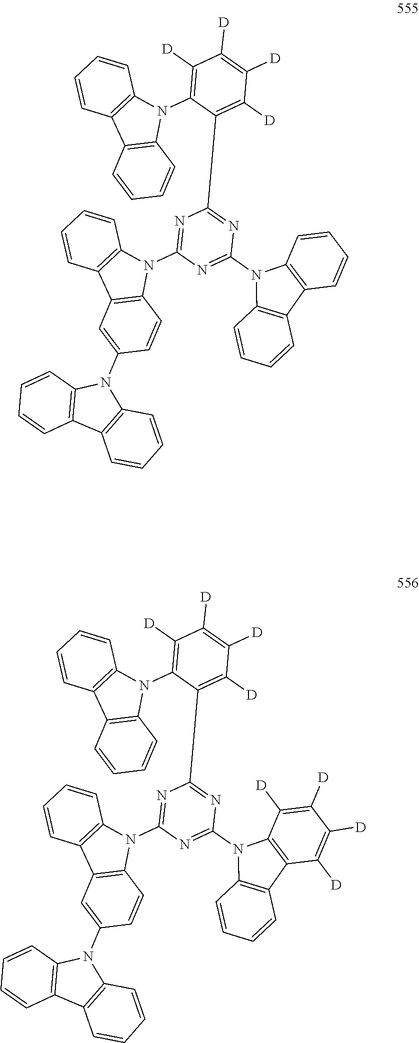 [SMILES: [2H]c1c([2H])c([2H])c(-n2c3ccccc3c3ccccc32)c(-c2nc(-n3c4ccccc4c4ccccc43)nc(-n3c4ccccc4c4cc(-n5c6ccccc6c6ccccc65)ccc43)n2)c1[2H] ; [2H]c1c([2H])c([2H])c(-n2c3ccccc3c3ccccc32)c(-c2nc(-n3c4ccccc4c4cc(-n5c6ccccc6c6ccccc65)ccc43)nc(-n3c4ccccc4c4c([2H])c([2H])c([2H])c([2H])c43)n2)c1[2H]] 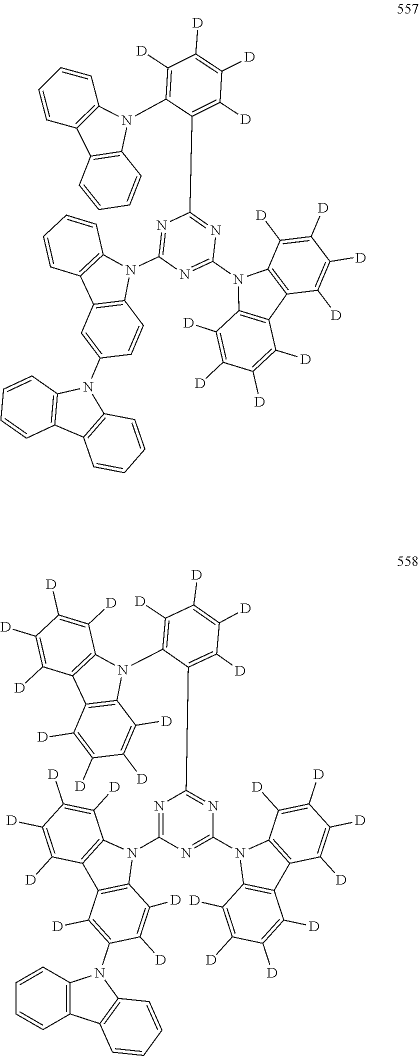 [SMILES: [2H]c1c([2H])c([2H])c(-n2c3ccccc3c3ccccc32)c(-c2nc(-n3c4ccccc4c4cc(-n5c6ccccc6c6ccccc65)ccc43)nc(-n3c4c([2H])c([2H])c([2H])c([2H])c4c4c([2H])c([2H])c([2H])c([2H])c43)n2)c1[2H] ; [2H]c1c([2H])c([2H])c(-n2c3c([2H])c([2H])c([2H])c([2H])c3c3c([2H])c([2H])c([2H])c([2H])c32)c(-c2nc(-n3c4c([2H])c([2H])c([2H])c([2H])c4c4c([2H])c([2H])c([2H])c([2H])c43)nc(-n3c4c([2H])c([2H])c([2H])c([2H])c4c4c([2H])c(-n5c6ccccc6c6ccccc65)c([2H])c([2H])c43)n2)c1[2H]] 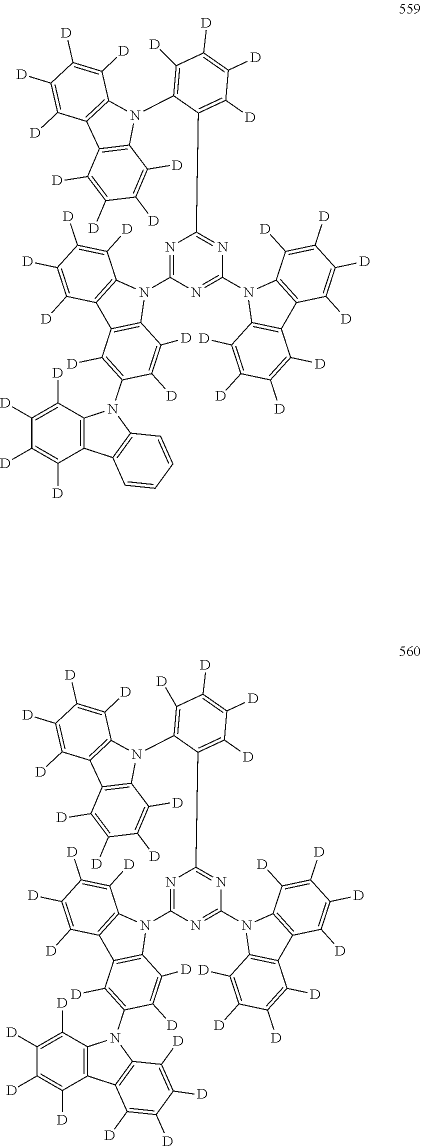 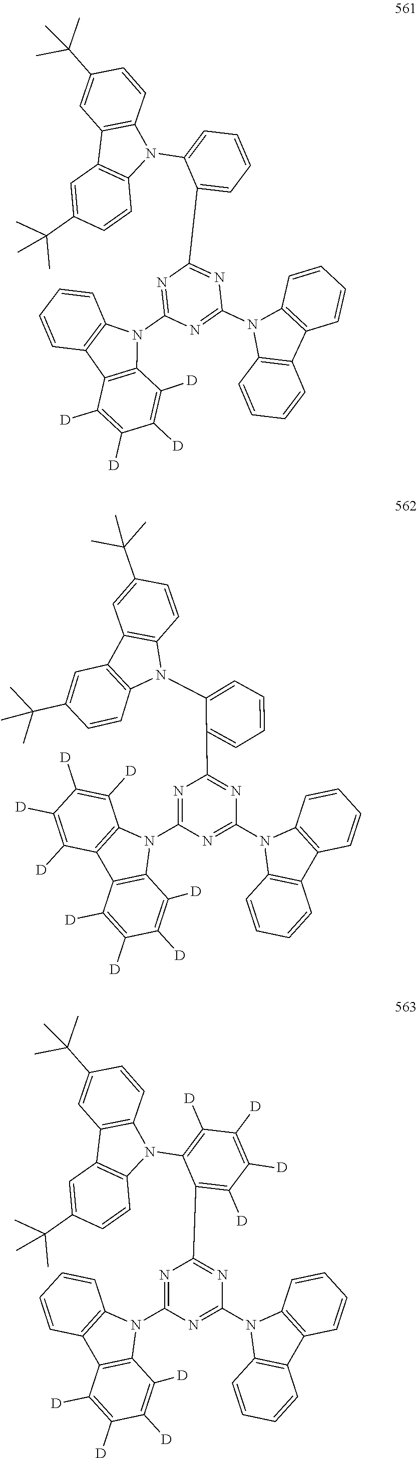 [SMILES: [2H]c1c([2H])c([2H])c2c(c1[2H])c1ccccc1n2-c1nc(-c2ccccc2-n2c3ccc(C(C)(C)C)cc3c3cc(C(C)(C)C)ccc32)nc(-n2c3ccccc3c3ccccc32)n1 ; [2H]c1c([2H])c([2H])c2c(c1[2H])c1c([2H])c([2H])c([2H])c([2H])c1n2-c1nc(-c2ccccc2-n2c3ccc(C(C)(C)C)cc3c3cc(C(C)(C)C)ccc32)nc(-n2c3ccccc3c3ccccc32)n1 ; [2H]c1c([2H])c([2H])c(-n2c3ccc(C(C)(C)C)cc3c3cc(C(C)(C)C)ccc32)c(-c2nc(-n3c4ccccc4c4ccccc43)nc(-n3c4ccccc4c4c([2H])c([2H])c([2H])c([2H])c43)n2)c1[2H]] 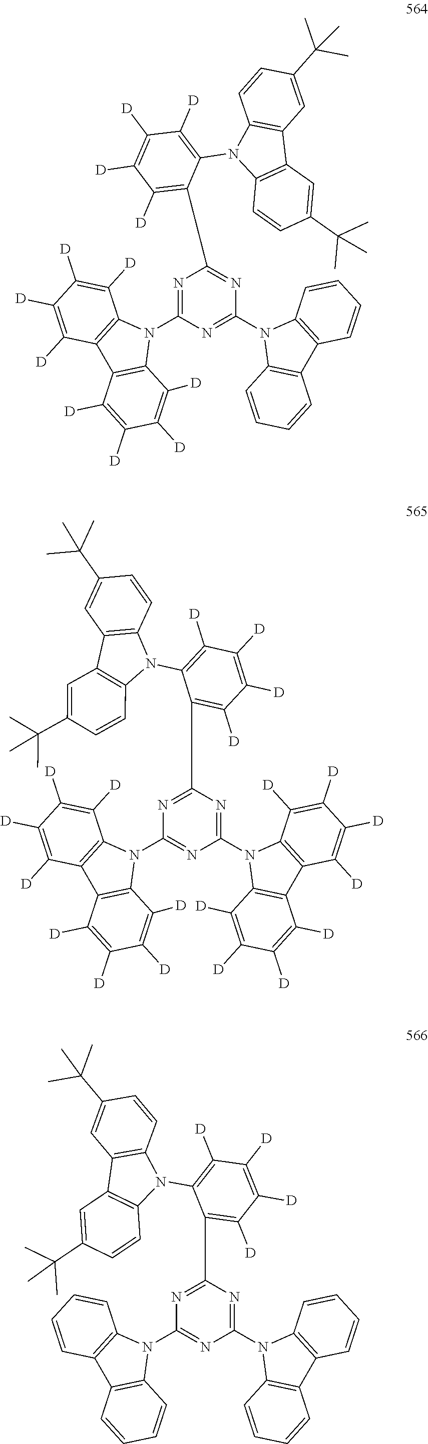 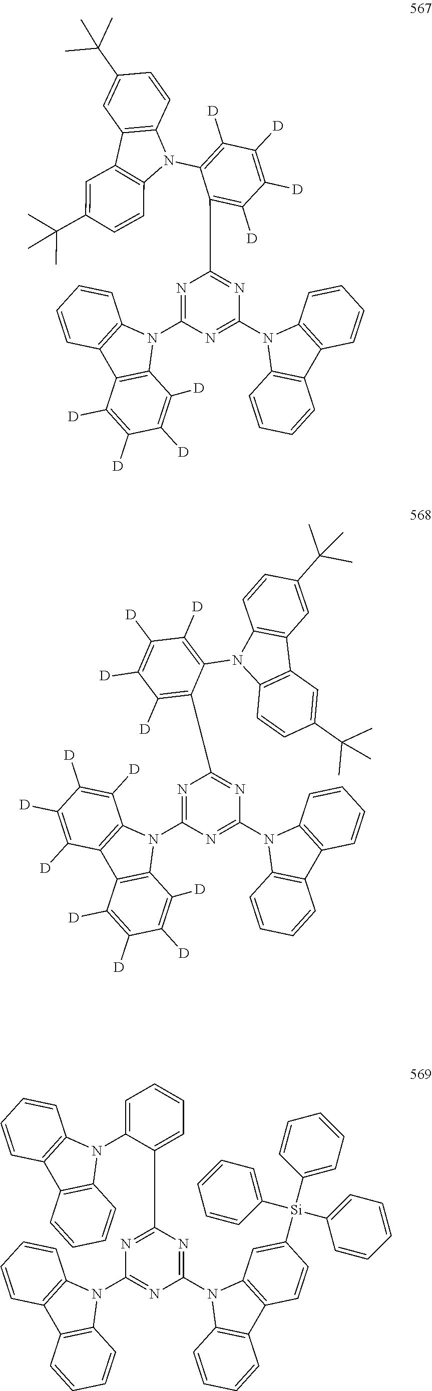 [SMILES: [2H]c1c([2H])c([2H])c(-n2c3ccc(C(C)(C)C)cc3c3cc(C(C)(C)C)ccc32)c(-c2nc(-n3c4ccccc4c4ccccc43)nc(-n3c4ccccc4c4c([2H])c([2H])c([2H])c([2H])c43)n2)c1[2H] ; [2H]c1c([2H])c([2H])c(-n2c3ccc(C(C)(C)C)cc3c3cc(C(C)(C)C)ccc32)c(-c2nc(-n3c4ccccc4c4ccccc43)nc(-n3c4c([2H])c([2H])c([2H])c([2H])c4c4c([2H])c([2H])c([2H])c([2H])c43)n2)c1[2H] ; c1ccc([Si](c2ccccc2)(c2ccccc2)c2ccc3c4ccccc4n(-c4nc(-c5ccccc5-n5c6ccccc6c6ccccc65)nc(-n5c6ccccc6c6ccccc65)n4)c3c2)cc1] 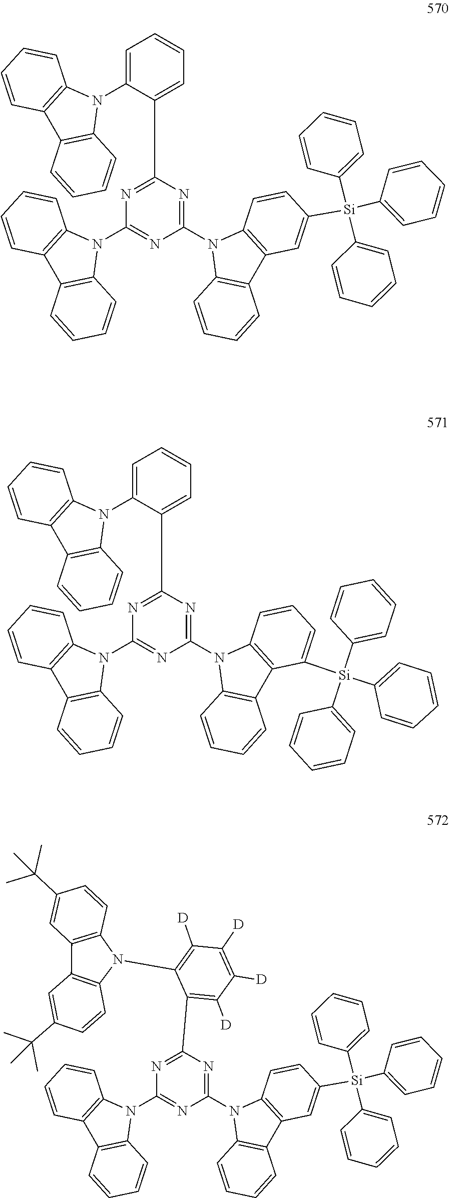 [SMILES: c1ccc([Si](c2ccccc2)(c2ccccc2)c2ccc3c(c2)c2ccccc2n3-c2nc(-c3ccccc3-n3c4ccccc4c4ccccc43)nc(-n3c4ccccc4c4ccccc43)n2)cc1 ; c1ccc([Si](c2ccccc2)(c2ccccc2)c2cccc3c2c2ccccc2n3-c2nc(-c3ccccc3-n3c4ccccc4c4ccccc43)nc(-n3c4ccccc4c4ccccc43)n2)cc1 ; [2H]c1c([2H])c([2H])c(-n2c3ccc(C(C)(C)C)cc3c3cc(C(C)(C)C)ccc32)c(-c2nc(-n3c4ccccc4c4ccccc43)nc(-n3c4ccccc4c4cc([Si](c5ccccc5)(c5ccccc5)c5ccccc5)ccc43)n2)c1[2H]] 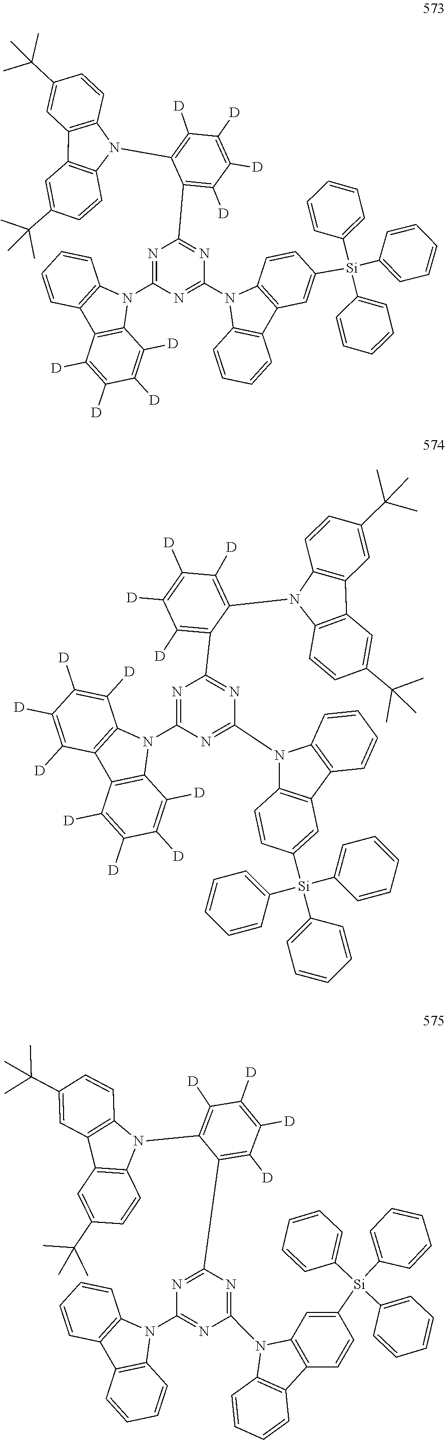 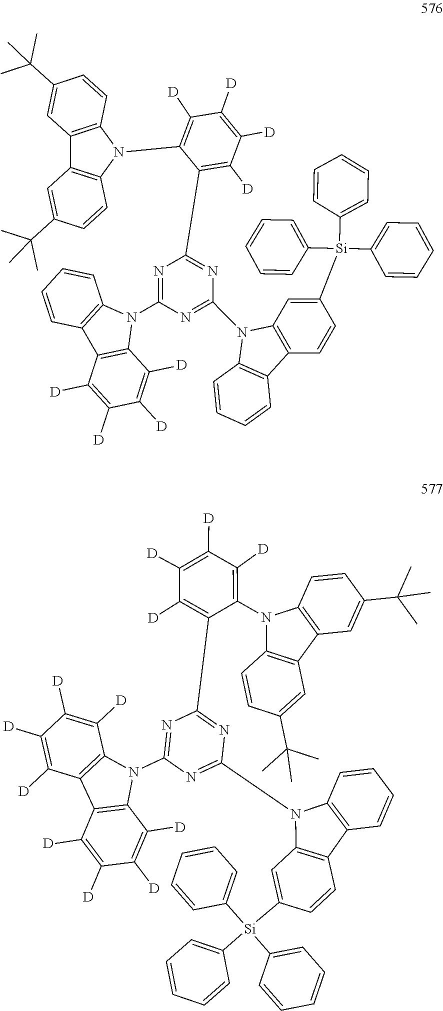 [SMILES: [2H]c1c([2H])c([2H])c(-n2c3ccc(C(C)(C)C)cc3c3cc(C(C)(C)C)ccc32)c(-c2nc(-n3c4ccccc4c4ccc([Si](c5ccccc5)(c5ccccc5)c5ccccc5)cc43)nc(-n3c4ccccc4c4c([2H])c([2H])c([2H])c([2H])c43)n2)c1[2H] ; [2H]c1c([2H])c([2H])c(-n2c3ccc(C(C)(C)C)cc3c3cc(C(C)(C)C)ccc32)c(-c2nc(-n3c4ccccc4c4ccc([Si](c5ccccc5)(c5ccccc5)c5ccccc5)cc43)nc(-n3c4c([2H])c([2H])c([2H])c([2H])c4c4c([2H])c([2H])c([2H])c([2H])c43)n2)c1[2H]] 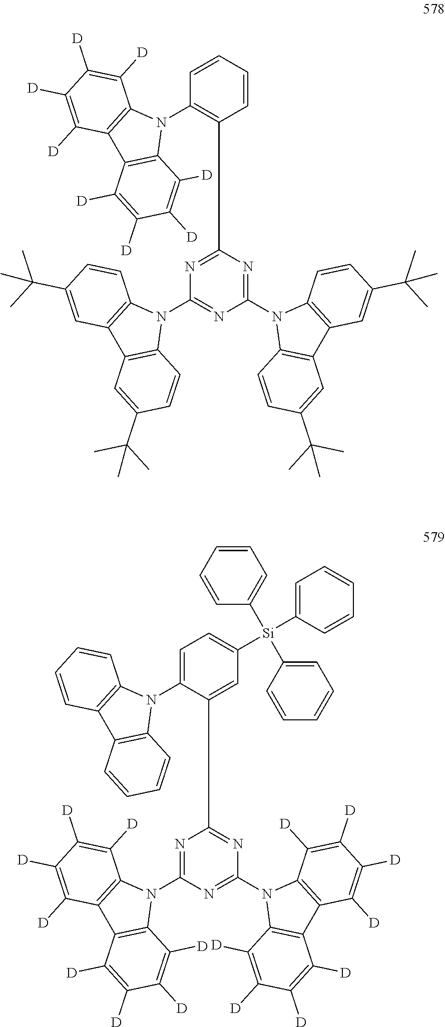 [SMILES: [2H]c1c([2H])c([2H])c2c(c1[2H])c1c([2H])c([2H])c([2H])c([2H])c1n2-c1ccccc1-c1nc(-n2c3ccc(C(C)(C)C)cc3c3cc(C(C)(C)C)ccc32)nc(-n2c3ccc(C(C)(C)C)cc3c3cc(C(C)(C)C)ccc32)n1 ; [2H]c1c([2H])c([2H])c2c(c1[2H])c1c([2H])c([2H])c([2H])c([2H])c1n2-c1nc(-c2cc([Si](c3ccccc3)(c3ccccc3)c3ccccc3)ccc2-n2c3ccccc3c3ccccc32)nc(-n2c3c([2H])c([2H])c([2H])c([2H])c3c3c([2H])c([2H])c([2H])c([2H])c32)n1] 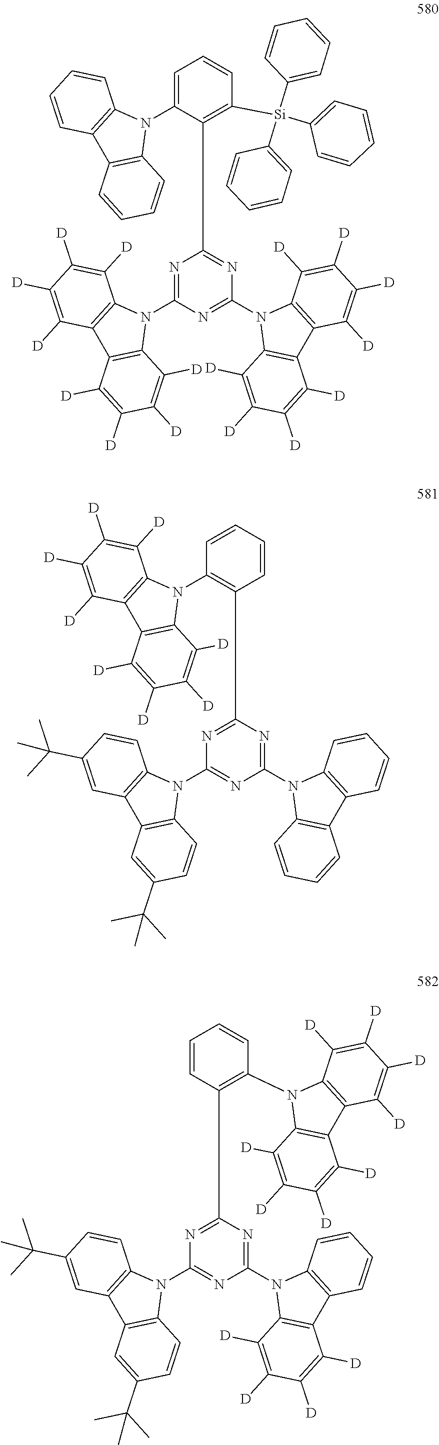 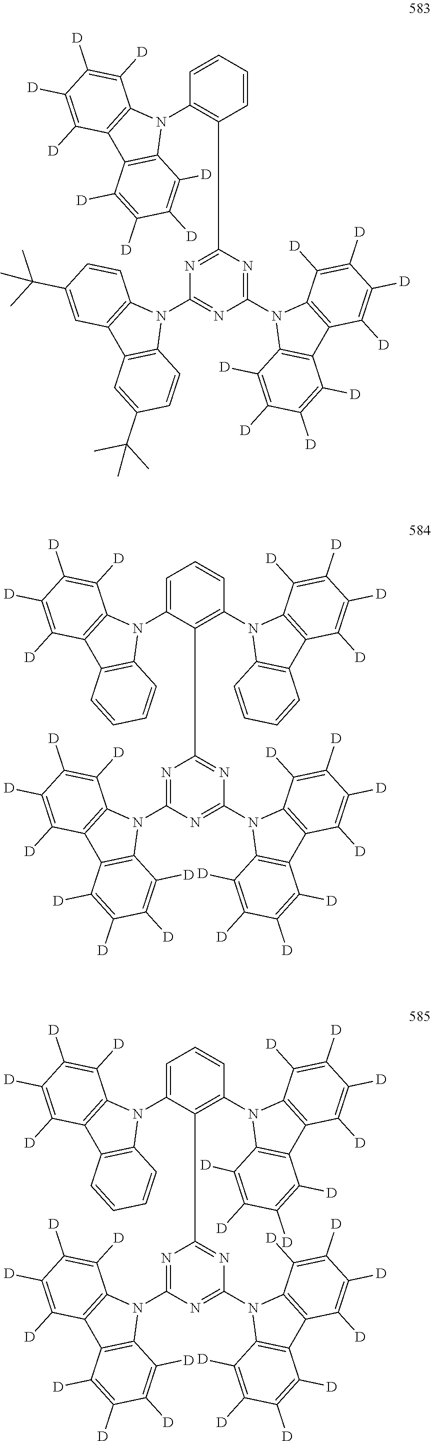 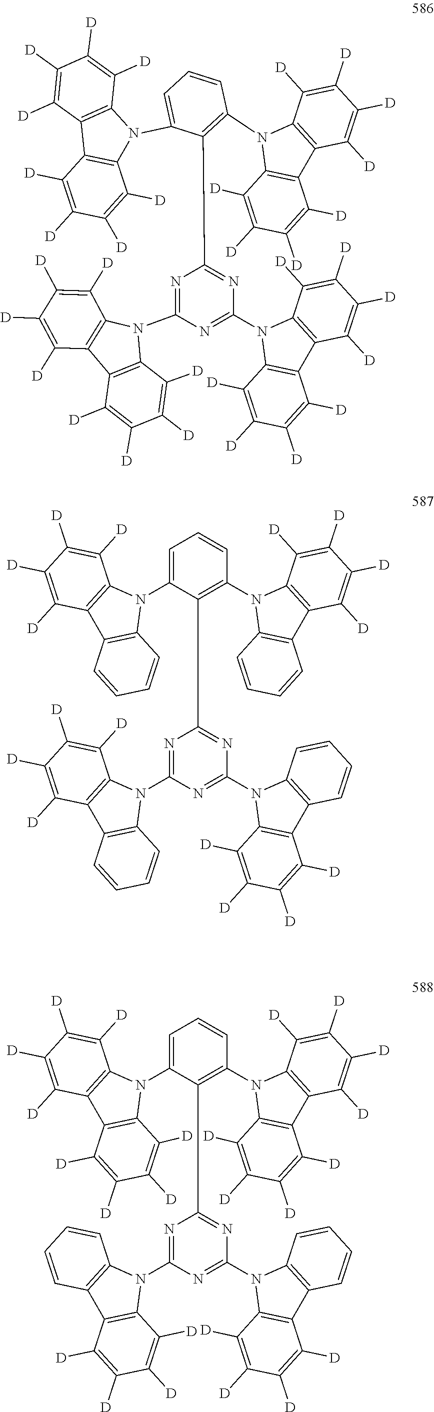 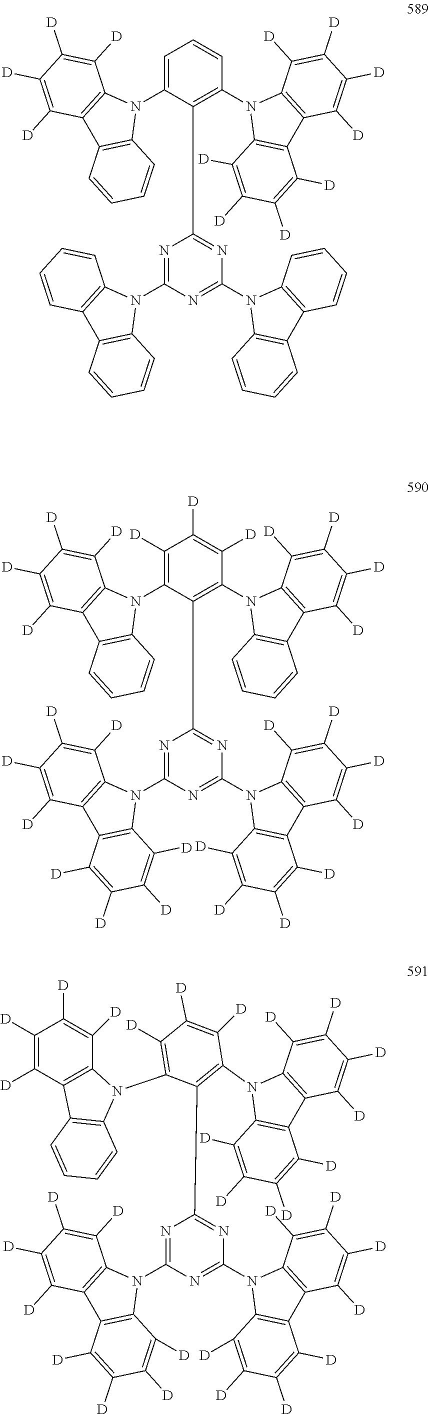 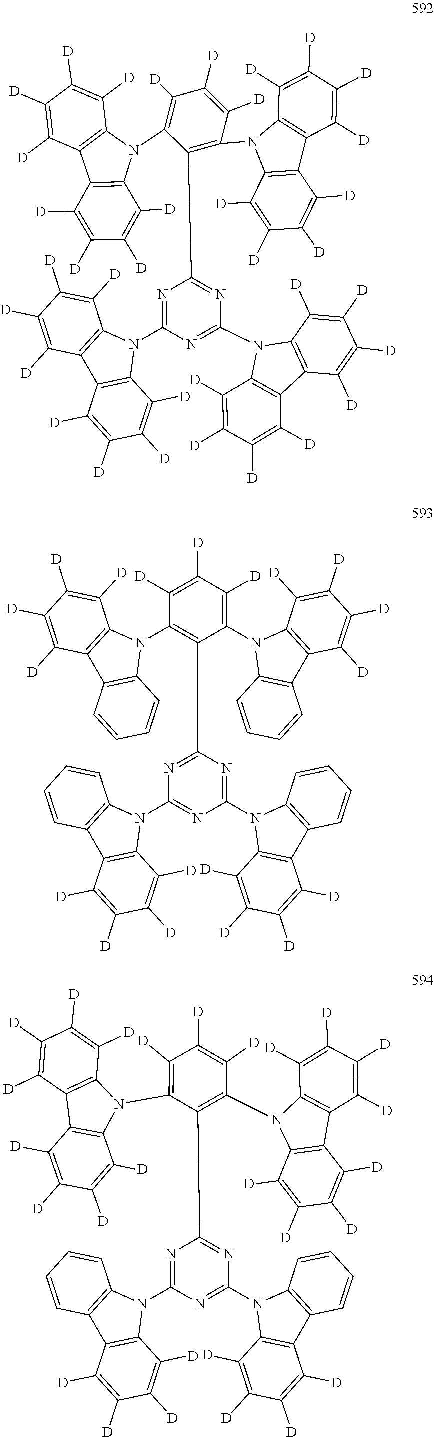 [chemical structure]
